(19) United States
(12) Patent Application Publication
(10) Pub. No.: US 2024/0287517 A1
(43) Pub. Date: Aug. 29, 2024

(54) TREATMENT OF PLIN1 RELATED DISEASES AND DISORDERS

(71) Applicant: Empirico Inc., San Diego, CA (US)

(72) Inventors: Omri GOTTESMAN, San Diego, CA (US); Shannon BRUSE, San Diego, CA (US); Paul BUSKE, Madison, WI (US); Brian CAJES, San Diego, CA (US); David JAKUBOSKY, San Diego, CA (US); Sarah KLEINSTEIN, San Diego, CA (US); David LEWIS, Madison, WI (US); David ROZEMA, Cross Plains, WI (US); John VEKICH, San Diego, CA (US)

(21) Appl. No.: 18/568,170

(22) PCT Filed: Jun. 14, 2022
(86) PCT No.: PCT/US2022/033479

§ 371 Date: Dec. 7, 2023

Related U.S. Application Data
(60) Provisional application No. 63/211,374, filed on Jun. 16, 2021.
(60) Provisional application No. 63/324,460, filed on Mar. 28, 2022.

(51) Int. Cl. C12N 15/113; A61P 3/06

(52) U.S. Cl. CPC C12N 15/113; A61P 3/06; C12N 2310/14; C12N 2310/315; C12N 2310/321; C12N 2310/322

(57) ABSTRACT

Disclosed herein are compositions comprising an oligonucleotide that targets PLIN1. The oligonucleotide may include a small interfering RNA (siRNA) or an antisense oligonucleotide (ASO). Also provided herein are methods of treating conditions associated with PLIN1 gene mutations that include providing an oligonucleotide that targets PLIN1 in a subject.

### CROSS-REFERENCE

This application claims the benefit of U.S. Provisional Application No, 63/211,374, filed Jun. 16, 2021, and U.S. Provisional Application No, 63/324,460, filed Mar. 28, 2022, which applications are incorporated herein by reference in their entireties.

### BACKGROUND

Cardiometabolic disorders are becoming increasingly abundant, and may affect a wide variety of people. Improved therapeutics are needed for treating these disorders.

### SUMMARY

Described herein are compositions comprising an oligonucleotide that targets a perilipin. Described herein are compositions comprising an oligonucleotide that targets a perilipin and when administered to a subject in an effective amount reduces a perilipin mRNA or protein level. The perilipin may include perilipin 1 (PLIN1). Described herein are compositions comprising an oligonucleotide that targets PLIN1 and when administered to a subject in an effective amount reduces a PLIN1 mRNA or protein level. Described herein, in some embodiments, are compositions comprising an oligonucleotide that targets PLIN1 and when administered to a subject in an effective amount decreases circulating cholesterol, triglycerides, glucose, hemoglobin A1c, apolipoprotein B (APOB), alanine aminotransferase, aspartate aminotransferase, alkaline phosphatase, or gamma-glutamyl transferase in a subject. In some embodiments, the cholesterol comprises total cholesterol, low density lipoprotein cholesterol, or non-high density lipoprotein cholesterol. In some embodiments, the decrease is by about 10% or more, as compared to prior to administration. Described herein, in some embodiments, are compositions comprising an oligonucleotide that targets PLIN1 and when administered to a subject in an effective amount decreases systolic blood pressure or diastolic blood pressure in a subject. In some embodiments, the decrease is by about 10% or more, as compared to prior to administration. Described herein, in some embodiments, are compositions comprising an oligonucleotide that targets PLIN1 and when administered to a subject in an effective amount decreases a liver fibrosis score, non-alcoholic fatty liver disease (NAFLD) fibrosis score. NAFLD activity score, or liver fat percentage in a subject. In some embodiments, the decrease is by about 10% or more, as compared to prior to administration. Described herein, in some embodiments, are compositions comprising an oligonucleotide that targets PLIN1 and when administered to a subject in an effective amount increases circulating high density lipoprotein cholesterol or apolipoprotein A1 in a subject. In some embodiments, the increase is by about 10% or more, as compared to prior to administration. Described herein, in some embodiments, are compositions comprising an oligonucleotide that targets PLIN1 and when administered to a subject in an effective amount increases left ventricular ejection fraction in a subject. In some embodiments, the increase is by about 10% or more, as compared to prior to administration. Described herein, in some embodiments, are compositions comprising an oligonucleotide that targets PLIN1 and when administered to a subject in an effective amount increases insulin sensitivity in a subject. In some embodiments, the increase is by about 10% or more, as compared to prior to administration. In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. In some embodiments, the oligonucleotide comprises 1, 2, 3, 4, 5, 6, 7, 8, 9, 10, 11, 12, 13, 14, 15, 16, 17, 18, 19, or 20 modified internucleoside linkages. In some embodiments, the oligonucleotide comprises a modified nucleoside. In some embodiments, the modified nucleoside comprises a locked nucleic acid (LNA), hexitol nucleic acid (HLA), cyclohexene nucleic acid (CeNA), 2′-methoxyethyl. 2′-O-alkyl, 2′-O-allyl, 2′-O-allyl, 2′-fluoro, or 2′-deoxy, or a combination thereof. In some embodiments, the modified nucleoside comprises a LNA. In some embodiments, the modified nucleoside comprises a 2′,4′ constrained ethyl nucleic acid. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises one or more 2′ fluoro modified nucleosides. In some embodiments, the modified nucleoside comprises a 2′ O-alkyl modified nucleoside. In some embodiments, the oligonucleotide comprises 1, 2, 3, 4, 5, 6, 7, 8, 9, 10, 11, 12, 13, 14, 15, 16, 17, 18, 19, 20, or 21 modified nucleosides. In some embodiments, the oligonucleotide comprises a lipid attached at a 3′ or 5′ terminus of the oligonucleotide. In some embodiments, the lipid comprises cholesterol, myristoyl, palmitoyl, stearoyl, lithocholyl, docosanoyl, docosahexaenoyl, myristyl, palmityl stearyl, or α-tocopherol, or a combination thereof. In some embodiments, the oligonucleotide comprises a sugar moiety attached at a 3′ or 5′ terminus of the oligonucleotide. In some embodiments, the sugar comprises N-acetylgalactosamine (GalNAc), N-acetylglucosamine (GlcNAc), or mannose. In some embodiments, the oligonucleotide comprises a small interfering RNA (siRNA) comprising a sense strand and an antisense strand. In some embodiments, the sense strand is 12-30 nucleosides in length. In some embodiments, the antisense strand is 12-30 nucleosides in length. Described herein, in some embodiments, are compositions comprising an oligonucleotide that inhibits the expression of PLIN1, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of SEQ ID NO: 6014. In some embodiments, any one of the following is true with regard to the sense strand: all purines comprise 2′ fluoro modified purines, and all pyrimidines comprise a mixture of 2′ fluoro and 2′ methyl modified pyrimidines; all purines comprise 2′ methyl modified purines, and all pyrimidines comprise a mixture of 2° fluoro and 2′ methyl modified pyrimidines; all purines comprise 2′ fluoro modified purines, and all pyrimidines comprise 2′ methyl modified pyrimidines; all pyrimidines comprise 2′ fluoro modified pyrimidines, and all purines comprise a mixture of 2′ fluoro and 2′ methyl modified purines; all pyrimidines comprise 2′ methyl modified pyrimidines, and all purines comprise a mixture of 2′ fluoro and 2′ methyl modified purines; or all pyrimidines comprise 2′ fluoro modified pyrimidines, and all purines comprise 2′ methyl modified purines. In some embodiments, the sense strand comprises any one of modification patterns 1S, 2S. 3S, 4S, 5S, 6S, 7S, 8S, 9S, 10S, 11S, 12S, 13S, 14S, 15S, 16S, 17S, 18S, 19S, 20S, 21S, 22S, 23S. 24S, 25S, 26S, 27S, 28S, 29S, 30S, 31S, 32S, 33S, 34S, 35S, 36S, 37S, 38S, 39S, 40S, 41S, 42S, 43S. 44S, 45S, 46S, 47S, 48S, 49S, or 50S. In some embodiments, any one of the following is true with regard to the antisense strand; all purines comprise 2′ fluoro modified purines, and all pyrimidines comprise a mixture of 2′ fluoro and 2′ methyl modified pyrimidines; all purines comprise 2′ methyl modified purines, and all pyrimidines comprise a mixture of 2′ fluoro and 2′ methyl modified pyrimidines; all purines comprise 2′ methyl modified purines, and all pyrimidines comprise 2′ fluoro modified pyrimidines; all pyrimidines comprise 2′ fluoro modified pyrimidines, and all purines comprise a mixture of 2′ fluoro and 2′ methyl modified purines; all pyrimidines comprise 2′ methyl modified pyrimidines, and all purines comprise a mixture of 2′ fluoro and 2′ methyl modified purines; or all pyrimidines comprise 2′ methyl modified pyrimidines, and all purines comprise 2′ fluoro modified purines. In some embodiments, the antisense strand comprises any one of modification patterns 1AS, 2AS, 3AS, 4AS, 5AS, 6AS, 7AS, 8AS, 9AS, or 10AS. In some embodiments, the sense strand comprises the nucleic acid sequence of any one of SEQ ID NOs: 1-2898, and the antisense strand comprises the nucleic acid sequence of any one of SEQ ID NOs: 2899-5796. In some embodiments, the oligonucleotide comprises an antisense oligonucleotide (ASO). In some embodiments, the ASO is 12-30 nucleosides in length. Described herein, in some embodiments, are compositions comprising an oligonucleotide that inhibits the expression of PLIN1, wherein the oligonucleotide comprises an ASO about 12-30 nucleosides in length and a nucleoside sequence complementary to about 12-30 contiguous nucleosides of SEQ ID NO: 6014. Some embodiments include a pharmaceutically acceptable carrier. Described herein, in some embodiments, are methods of treating a subject having a cardiometabolic disorder, comprising administering an effective amount of the composition to the subject. In some embodiments, the cardiometabolic disorder comprises hyperlipidemia, hypertriglyceridemia, cardiovascular disease, coronary artery disease, myocardial infarction, heart failure, cerebrovascular disease, peripheral vascular disease, peripheral arterial disease, stroke, hypertension, diabetes, NAFLD, or non-alcoholic steatohepatitis.

### BRIEF DESCRIPTION OF THE DRAWINGS

FIG. 1 is a PLIN1 western blot image.

### DETAILED DESCRIPTION

Large-scale human genetic data can improve the success rate of pharmaceutical discovery and development. A Genome Wide Association Study (GWAS) detects associations between genetic variants and traits in a population sample, and this improves understanding of the biology of disease and provides evidence of applicable treatments. A GWAS generally utilizes genotyping and/or sequencing data, and often involves an evaluation of millions of genetic variants that are relatively evenly distributed across the genome. The most common GWAS design is the case-control study, which involves comparing variant frequencies in cases versus controls. If a variant has a significantly different frequency in cases versus controls, that variant is considered associated with disease. Association statistics used in a GWAS include p-values, as a measure of statistical significance; odds ratios (OR), as a measure of effect size; or beta coefficients (beta), as a measure of effect size. Researchers often assume an additive genetic model and calculate an allelic odds ratio, which is the increased (or decreased) risk of disease conferred by each additional copy of an allele (compared to carrying no copies of that allele). An additional concept in design and interpretation of GWAS is that of linkage disequilibrium, which is the non-random association of alleles. The presence of linkage disequilibrium can obfuscate which variant is “causal.”

Functional annotation of variants and/or wet lab experimentation is used to identify a causal genetic variant identified via GWAS, and in many cases leads to identification of disease-causing genes. In particular, understanding the functional effect of a causal genetic variant (for example, loss of protein function, gain of protein function, increase in gene expression, or decrease in gene expression) allows that variant to be used as a proxy for therapeutic modulation of the target gene, or to gain insight into potential therapeutic efficacy and safety of a therapeutic that modulates that target.

Identification of such gene-disease associations has provided insights into disease biology and is used to identify novel therapeutic targets for the pharmaceutical industry. In order to translate the therapeutic insights derived from human genetics, disease biology in patients is exogenously ‘programmed’ into replicating the observation from human genetics. There are several options for therapeutic modalities that may be brought to bear in translating therapeutic targets identified via human genetics into novel medicines. These include well established therapeutic modalities such as small molecules and monoclonal antibodies, maturing modalities such as oligonucleotides, and emerging modalities such as gene therapy and gene editing. The choice of therapeutic modality depends on factors such as the location of a target (for example, intracellular, extracellular, or secreted), a relevant tissue (for example, lung or liver) and a relevant indication.

The PLIN1 gene is located on chromosome 15, and encodes perilipin 1 (PLIN1). PLIN1 may include 522 amino acids and have a mass of about 56 kDa. PLIN1 may be an intracellular protein. PLIN1 may associate with the surface of lipid droplets. Phosphorylation of PLIN1 may be involved in mobilization of fats in adipose tissue. PLIN1 may be expressed in liver cells such as hepatocytes, or in fat cells such as white adipocytes. Decreasing PLIN1 expression may increase lipolysis. Alternatively spliced mRNA transcript variants varying in the 5′ UTR, but encoding the same protein, have been found for PLIN1. An example of a PLIN1 amino acid sequence, and further description of PLIN1 is included at uniprot.org under accession no, 060240 (last modified May 5, 2009).

Here it is shown that genetic variant cause inactivation of PLIN1 result in protective associations for cardiometabolic phenotypes including circulating triglyceride levels, circulating high-density lipoprotein (HDL) levels, circulating low density lipoprotein (LDL) levels, statin medication use, myocardial infarction, angina, family history of stroke, and hypertension. Therefore, inhibition of PLIN1 may serve as a therapeutic for treatment of cardiometabolic diseases and disorders such as hyperlipidemia, hypertriglyceridemia, cardiovascular disease, coronary artery disease, peripheral vascular disease, peripheral arterial disease, myocardial infarction, heart failure, cerebrovascular disease, stroke, hypertension, diabetes, non-alcoholic fatty liver disease (NAFLD), or non-alcoholic steatohepatitis (NASH).

Disclosed herein are compositions comprising an oligonucleotide that targets PLIN1. Where inhibition or targeting of PLIN1 is disclosed, it is contemplated that some embodiments may include inhibiting or targeting a PLIN1 protein or PLIN1 RNA. For example, by inhibiting or targeting an RNA (e.g. mRNA) encoded by the PLIN1 gene using an oligonucleotide described herein, the PLIN1 protein may be inhibited or targeted as a result of there being less production of the PLIN1 protein by translation of the PLIN1 RNA; or a PLIN1 protein may be targeted or inhibited by an oligonucleotide that binds or interacts with a PLIN1 RNA and reduces production of the PLIN1 protein from the PLIN1 RNA. Thus, targeting PLIN1 may refer to binding a PLIN1 RNA and reducing PLIN1 RNA or protein levels. The oligonucleotide may include a small interfering RNA (siRNA) or an antisense oligonucleotide (ASO). Administration of the oligonucleotide to a subject may improve (e.g. decrease or increase, depending on the indication or parameter) total circulating cholesterol, circulating non-HDL cholesterol, circulating triglycerides, circulating LDL, circulating hemoglobin A1c, fasting circulating glucose, systolic blood pressure, diastolic blood pressure, circulating alanine aminotransferase (ALT), circulating aspartate aminotransferase (AST), blood alkaline phosphatase (ALP), gamma-glutamyl transferase (GGT). NAFLD fibrosis score, liver fat percentage, liver fibrosis score, or NAFLD activity score, or increase circulating HDL, circulating apolipoprotein A1 (ApoA1), circulating APOB, left ventricular ejection fraction, or insulin sensitivity in the subject. Also provided herein are methods of treating a cardiometabolic disorder by providing an oligonucleotide that targets PLIN1 to a subject in need thereof.

### I. Compositions

Disclosed herein, in some embodiments, are compositions comprising an oligonucleotide. In some embodiments, the composition comprises an oligonucleotide that targets PLIN1. In some embodiments, the composition consists of an oligonucleotide that targets PLIN1. In some embodiments, the oligonucleotide reduces PLIN1 mRNA expression in the subject. In some embodiments, the oligonucleotide reduces PLIN1 protein expression in the subject. The oligonucleotide may include a small interfering RNA (siRNA) described herein. The oligonucleotide may include an antisense oligonucleotide (ASO) described herein. In some embodiments, a composition described herein is used in a method of treating a disorder in a subject in need thereof. Some embodiments relate to a composition comprising an oligonucleotide for use in a method of treating a disorder as described herein. Some embodiments relate to use of a composition comprising an oligonucleotide, in a method of treating a disorder as described herein.

Some embodiments include a composition comprising an oligonucleotide that targets PLIN1 and when administered to a subject in an effective amount decreases PLIN1 mRNA or protein levels in a cell, fluid or tissue. In some embodiments, the composition comprises an oligonucleotide that targets PLIN1 and when administered to a subject in an effective amount decreases PLIN1 mRNA levels in a cell or tissue. In some embodiments, the cell is a hepatocyte. In some embodiments, the cell is an adipocyte. In some embodiments, the cell is an preadipocyte. In some embodiments, the tissue is liver tissue. In some embodiments, the tissue is adipose tissue. In some embodiments, the PLIN1 mRNA levels are decreased by about 2.5% or more, about 5% or more, or about 7.5% or more, as compared to prior to administration. In some embodiments, the PLIN1 mRNA levels are decreased by about 10% or more, as compared to prior to administration. In some embodiments, the PLIN1 mRNA levels are decreased by about 20% or more, about 30% or more, about 40% or more, about 50% or more, about 60% or more, about 70% or more, about 80% or more, about 90% or more, or about 100%, as compared to prior to administration. In some embodiments, the PLIN1 mRNA levels are decreased by no more than about 2.5%, no more than about 5%, or no more than about 7.5%, as compared to prior to administration. In some embodiments, the PLIN1 mRNA levels are decreased by no more than about 10%, as compared to prior to administration. In some embodiments, the PLIN1 mRNA levels are decreased by no more than about 20%, no more than about 30%, no more than about 40%, no more than about 50%, no more than about 60%, no more than about 70%, no more than about 80%, or no more than about 90%, as compared to prior to administration. In some embodiments, the PLIN1 mRNA levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets PLIN1 and when administered to a subject in an effective amount decreases PLIN1 protein levels in a cell, fluid or tissue. In some embodiments, the cell is a hepatocyte. In some embodiments, the cell is an adipocyte. In some embodiments, the tissue is liver tissue. In some embodiments, the tissue is adipose tissue. In some embodiments, the PLIN1 protein levels are decreased by about 2.5% or more, about 5% or more, or about 7.5% or more, as compared to prior to administration. In some embodiments, the PLIN1 protein levels are decreased by about 10% or more, as compared to prior to administration. In some embodiments, the PLIN1 protein levels are decreased by about 20% or more, about 30% or more, about 40% or more, about 50% or more, about 60% or more, about 70% or more, about 80% or more, about 90% or more, or about 100%, as compared to prior to administration. In some embodiments, the PLIN1 protein levels are decreased by no more than about 2.5%, no more than about 5%, or no more than about 7.5%, as compared to prior to administration. In some embodiments the PLIN1 protein levels are decreased by no more than about 10%, as compared to prior to administration. In some embodiments, the PLIN1 protein levels are decreased by no more than about 20%, no more than about 30%, no more than about 40%, no more than about 50%, no more than about 60%, no more than about 70%, no more than about 80%, or no more than about 90%, as compared to prior to administration. In some embodiments, the PLIN1 protein levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets PLIN1 and when administered to a subject in an effective amount diminishes a cardiometabolic disease phenotype. The cardiometabolic disease may include hyperlipidemia, hypertriglyceridemia, cardiovascular disease, coronary artery disease, myocardial infarction, heart failure, cerebrovascular disease, stroke, hypertension, diabetes, non-alcoholic fatty liver disease (NAFLD), or non-alcoholic steatohepatitis (NASH). In some embodiments, the cardiometabolic disease phenotype is decreased by about 2.5% or more, about 5% or more, or about 7.5% or more, as compared to prior to administration. In some embodiments, the cardiometabolic disease phenotype is decreased by about 10% or more, as compared to prior to administration. In some embodiments, the cardiometabolic disease phenotype is decreased by about 20% or more, about 30% or more, about 40% or more, about 50% or more, about 60% or more, about 70% or more, about 80% or more, about 90% or more, or about 100%, as compared to prior to administration. In some embodiments, the cardiometabolic disease phenotype is decreased by no more than about 2.5%, no more than about 5%, or no more than about 7.5%, as compared to prior to administration. In some embodiments, the cardiometabolic disease phenotype is decreased by no more than about 10%, as compared to prior to administration. In some embodiments, the cardiometabolic disease phenotype is decreased by no more than about 20%, no more than about 30%, no more than about 40%, no more than about 50%, no more than about 60%, no more than about 70%, no more than about 80%, or no more than about 90%, as compared to prior to administration. In some embodiments, the cardiometabolic disease phenotype is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets PLIN1 and when administered to a subject in an effective amount enhances a protective phenotype against a cardiometabolic disease in the subject. The cardiometabolic disease may include hyperlipidemia, hypertriglyceridemia, cardiovascular disease, coronary artery disease, myocardial infarction, heart failure, cerebrovascular disease, stroke, hypertension, diabetes, non-alcoholic fatty liver disease (NAFLD), or non-alcoholic steatohepatitis (NASH). In some embodiments, the protective phenotype is increased by about 2.5% or more, about 5% or more, or about 7.5% or more, as compared to prior to administration. In some embodiments, the protective phenotype is increased by about 10% or more as compared to prior to administration. In some embodiments, the protective phenotype is increased by about 20% or more, about 30% or more, about 40% or more, about 50% or more, about 60% or more, about 70% or more, about 80% or more about 90% or more, or about 100% or more, as compared to prior to administration. In some embodiments, the protective phenotype is increased by about 200% or more, about 300% or more, about 400% or more, about 500% or more, about 600% or more, about 700% or more, about 800% or more about 900% or more, or about 1000% or more, as compared to prior to administration. In some embodiments, the protective phenotype is increased by no more than about 2.5%, no more than about 5%, or no more than about 7.5%, as compared to prior to administration. In some embodiments, the protective phenotype is increased by no more than about 10%, as compared to prior to administration. In some embodiments, the protective phenotype is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the protective phenotype is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the protective phenotype is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

In some embodiments, the composition comprises an oligonucleotide that targets PLIN1 and when administered to a subject in an effective amount decreases circulating cholesterol in the subject. The circulating cholesterol may include total cholesterol or non-high density lipoprotein (HDL) cholesterol. The circulating cholesterol may include total cholesterol. The circulating cholesterol may include non-HDL cholesterol. In some embodiments, the circulating cholesterol is decreased by about 2.5% or more, about 5% or more, or about 7.5% or more, as compared to prior to administration. In some embodiments, the circulating cholesterol is decreased by about 10% or more, as compared to prior to administration. In some embodiments, the circulating cholesterol is decreased by about 20% or more, about 30% or more, about 40% or more, about 50% or more, about 60% or more, about 70% or more, about 80% or more, or about 90% or more, as compared to prior to administration. In some embodiments, the circulating cholesterol is decreased by no more than about 2.5%, no more than about 5%, or no more than about 7.5%, as compared to prior to administration. In some embodiments, the circulating cholesterol is decreased by no more than about 10%, as compared to prior to administration. In some embodiments, the circulating cholesterol is decreased by no more than about 20%, no more than about 30%, no more than about 40%, no more than about 50%, no more than about 60%, no more than about 70%, no more than about 80%, or no more than about 90%, as compared to prior to administration. In some embodiments, the circulating cholesterol is decreased by 2.5%, 5%, 7.5%, 10%, 15%, 20%, 25%, 30%, 35%, 40%, 45%, 50%, 55%, 60%, 65%, 70%, 75%, 80%, 85%, or 90%, or by a range defined by any of the two aforementioned percentages.

In some embodiments, the composition comprises an oligonucleotide that targets PLIN1 and when administered to a subject in an effective amount decreases circulating low density lipoproteins (LDL) in the subject. In some embodiments, the circulating LDL is decreased by about 2.5% or more, about 5% or more, or about 7.5% or more, as compared to prior to administration. In some embodiments, the circulating LDL is decreased by about 10% or more, as compared to prior to administration. In some embodiments, the circulating LDL is decreased by about 20% or more, about 30% or more, about 40% or more, about 50% or more, about 60% or more, about 70% or more, about 80% or more, or about 90% or more, as compared to prior to administration. In some embodiments, the circulating LDL is decreased by no more than about 2.5%, no more than about 5%, or no more than about 7.5%, as compared to prior to administration. In some embodiments, the circulating LDL is decreased by no more than about 10%, as compared to prior to administration. In some embodiments, the circulating LDL is decreased by no more than about 20%, no more than about 30%, no more than about 40%, no more than about 50%, no more than about 60%, no more than about 70%, no more than about 80%, or no more than about 90%, as compared to prior to administration. In some embodiments, the circulating LDL is decreased by 2.5%, 5%, 7.5%, 10%, 15%, 20%, 25%, 30%, 35%, 40%, 45%, 50%, 55%, 60%, 65%, 70%, 75%, 80%, 85%, or 90%, or by a range defined by any of the two aforementioned percentages.

In some embodiments, the composition comprises an oligonucleotide that targets PLIN1 and when administered to a subject in an effective amount decreases circulating triglycerides in the subject. In some embodiments, the circulating triglycerides are decreased by about 2.5% or more, about 5% or more, or about 7.5% or more, as compared to prior to administration. In some embodiments, the circulating triglycerides are decreased by about 10% or more, as compared to prior to administration. In some embodiments, the circulating triglycerides are decreased by about 20% or more, about 30% or more, about 40% or more, about 50% or more, about 60% or more, about 70% or more, about 80% or more, or about 90% or more, as compared to prior to administration. In some embodiments, the circulating triglycerides are decreased by no more than about 2.5%, no more than about 5%, or no more than about 7.5%, as compared to prior to administration. In some embodiments, the circulating triglycerides are decreased by no more than about 10%, as compared to prior to administration. In some embodiments, the circulating triglycerides are decreased by no more than about 20%, no more than about 30%, no more than about 40%, no more than about 50%, no more than about 60%, no more than about 70%, no more than about 80%, or no more than about 90%, as compared to prior to administration. In some embodiments, the circulating triglycerides are decreased by 2.5%, 5%, 7.5%, 10%, 15%, 20%, 25%, 30%, 35%, 40%, 45%, 50%, 55%, 60%, 65%, 70%, 75%, 80%, 85%, or 90%, or by a range defined by any of the two aforementioned percentages.

In some embodiments, the composition comprises an oligonucleotide that targets PLIN1 and when administered to a subject in an effective amount decreases circulating hemoglobin A1c in the subject. In some embodiments, the circulating hemoglobin A1c is decreased by about 2.5% or more, about 5% or more, or about 7.5% or more, as compared to prior to administration. In some embodiments, the circulating hemoglobin A1c is decreased by about 10% or more, as compared to prior to administration. In some embodiments, the circulating hemoglobin A1c is decreased by about 20% or more, about 30% or more, about 40% or more, about 50% or more, about 60% or more, about 70% or more, about 80% or more, or about 90% or more, as compared to prior to administration. In some embodiments, the circulating hemoglobin A1c is decreased by no more than about 2.5%, no more than about 5%, or no more than about 7.5%, as compared to prior to administration. In some embodiments, the circulating hemoglobin A1c is decreased by no more than about 10%, as compared to prior to administration. In some embodiments, the circulating hemoglobin A1c is decreased by no more than about 20%, no more than about 30%, no more than about 40%, no more than about 50%, no more than about 60%, no more than about 70%, no more than about 80%, or no more than about 90%, as compared to prior to administration. In some embodiments, the circulating hemoglobin A1c is decreased by 2.5%, 5%, 7.5%, 10%, 15%, 20%, 25%, 30%, 35%, 40%, 45%, 50%, 55%, 60%, 65%, 70%, 75%, 80%, 85%, or 90%, or by a range defined by any of the two aforementioned percentages.

In some embodiments, the composition comprises an oligonucleotide that targets PLIN1 and when administered to a subject in an effective amount decreases circulating Apolipoprotein B (APOB) in the subject. In some embodiments, the circulating APOB is decreased by about 2.5% or more, about 5% or more, or about 7.5% or more, as compared to prior to administration. In some embodiments, the circulating APOB is decreased by about 10% or more, as compared to prior to administration. In some embodiments, the circulating APOB is decreased by about 20% or more, about 30% or more, about 40% or more, about 50% or more, about 60% or more, about 70% or more, about 80% or more, or about 90% or more, as compared to prior to administration. In some embodiments, the circulating APOB is decreased by no more than about 2.5%, no more than about 5%, or no more than about 7.5%, as compared to prior to administration. In some embodiments, the circulating APOB is decreased by no more than about 10%, as compared to prior to administration. In some embodiments, the circulating APOB is decreased by no more than about 20%, no more than about 30%, no more than about 40%, no more than about 50%, no more than about 60%, no more than about 70%, no more than about 80%, or no more than about 90%, as compared to prior to administration. In some embodiments, the circulating APOB is decreased by 2.5%, 5%, 7.5%, 10%, 15%, 20%, 25%, 30%, 35%, 40%, 45%, 50%, 55%, 60%, 65%, 70%, 75%, 80%, 85%, or 90%, or by a range defined by any of the two aforementioned percentages.

In some embodiments, the composition comprises an oligonucleotide that targets PLIN1 and when administered to a subject in an effective amount decreases circulating glucose in the subject. The circulating glucose may be fasting circulating glucose. In some embodiments, the circulating glucose is decreased by about 2.5% or more, about 5% or more, or about 7.5% or more, as compared to prior to administration. In some embodiments, the circulating glucose is decreased by about 10% or more, as compared to prior to administration. In some embodiments, the circulating glucose is decreased by about 20% or more, about 30% or more, about 40% or more, about 50% or more, about 60% or more, about 70% or more, about 80% or more, or about 90% or more, as compared to prior to administration. In some embodiments, the circulating glucose is decreased by no more than about 2.5%, no more than about 5%, or no more than about 7.5%, as compared to prior to administration. In some embodiments, the circulating glucose is decreased by no more than about 10%, as compared to prior to administration. In some embodiments, the circulating glucose is decreased by no more than about 20%, no more than about 30%, no more than about 40%, no more than about 50%, no more than about 60%, no more than about 70%, no more than about 80%, or no more than about 90%, as compared to prior to administration. In some embodiments, the circulating glucose is decreased by 2.5%, 5%, 7.5%, 10%, 15%, 20%, 25%, 30%, 35%, 40%, 45%, 50%, 55%, 60%, 65%, 70%, 75%, 80%, 85%, or 90%, or by a range defined by any of the two aforementioned percentages.

In some embodiments, the composition comprises an oligonucleotide that targets PLIN1 and when administered to a subject in an effective amount decreases blood pressure in the subject. The blood pressure may be systolic blood pressure. The blood pressure may be diastolic blood pressure. In some embodiments, the blood pressure is decreased by about 2.5% or more, about 5% or more, or about 7.5% or more, as compared to prior to administration. In some embodiments, the blood pressure is decreased by about 10% or more, as compared to prior to administration. In some embodiments, the blood pressure is decreased by about 20% or more, about 30% or more, about 40% or more, about 50% or more, about 60% or more, about 70% or more, about 80% or more, or about 90% or more, as compared to prior to administration. In some embodiments, the blood pressure is decreased by no more than about 2.5%, no more than about 5%, or no more than about 7.5%, as compared to prior to administration. In some embodiments, the blood pressure is decreased by no more than about 10%, as compared to prior to administration. In some embodiments, the blood pressure is decreased by no more than about 20%, no more than about 30%, no more than about 40%, no more than about 50%, no more than about 60%, no more than about 70%, no more than about 80%, or no more than about 90%, as compared to prior to administration. In some embodiments, the blood pressure is decreased by 2.5%, 5%, 7.5%, 10%, 15%, 20%, 25%, 30%, 35%, 40%, 45%, 50%, 55%, 60%, 65%, 70%, 75%, 80%, 85%, or 90%, or by a range defined by any of the two aforementioned percentages.

In some embodiments, the composition comprises an oligonucleotide that targets PLIN1 and when administered to a subject in an effective amount decreases circulating alanine aminotransferase (ALT) in the subject. In some embodiments, the circulating ALT is decreased by about 2.5% or more, about 5% or more, or about 7.5% or more, as compared to prior to administration. In some embodiments, the circulating ALT is decreased by about 10% or more, as compared to prior to administration. In some embodiments, the circulating ALT is decreased by about 20% or more, about 30% or more, about 40% or more, about 50% or more, about 60% or more, about 70% or more, about 80% or more, or about 90% or more, as compared to prior to administration. In some embodiments, the circulating ALT is decreased by no more than about 2.5%, no more than about 5%, or no more than about 7.5%, as compared to prior to administration. In some embodiments, the circulating ALT is decreased by no more than about 10%, as compared to prior to administration. In some embodiments, the circulating ALT is decreased by no more than about 20%, no more than about 30%, no more than about 40%, no more than about 50%, no more than about 60%, no more than about 70%, no more than about 80%, or no more than about 90%, as compared to prior to administration. In some embodiments, the circulating ALT is decreased by 2.5%, 5%, 7.5%, 10%, 15%, 20%, 25%, 30%, 35%, 40%, 45%, 50%, 55%, 60%, 65%, 70%, 75%, 80%, 85%, or 90%, or by a range defined by any of the two aforementioned percentages.

In some embodiments, the composition comprises an oligonucleotide that targets PLIN1 and when administered to a subject in an effective amount decreases circulating aspartate aminotransferase (AST) in the subject. In some embodiments, the circulating AST is decreased by about 2.5% or more, about 5% or more, or about 7.5% or more, as compared to prior to administration. In some embodiments, the circulating AST is decreased by about 10% or more, as compared to prior to administration. In some embodiments, the circulating AST is decreased by about 20% or more, about 30% or more, about 40% or more, about 50% or more, about 60% or more, about 70% or more, about 80% or more, or about 90% or more, as compared to prior to administration. In some embodiments, the circulating AST is decreased by no more than about 2.5%, no more than about 5%, or no more than about 7.5%, as compared to prior to administration. In some embodiments, the circulating AST is decreased by no more than about 10%, as compared to prior to administration. In some embodiments, the circulating AST is decreased by no more than about 20%, no more than about 30%, no more than about 40%, no more than about 50%, no more than about 60%, no more than about 70%, no more than about 80%, or no more than about 90%, as compared to prior to administration. In some embodiments, the circulating AST is decreased by 2.5%, 5%, 7.5%, 10%, 15%, 20%, 25%, 30%, 35%, 40%, 45%, 50%, 55%, 60%, 65%, 70%, 75%, 80%, 85%, or 90%, or by a range defined by any of the two aforementioned percentages.

In some embodiments, the composition comprises an oligonucleotide that targets PLIN1 and when administered to a subject in an effective amount decreases circulating alkaline phosphatase (ALP) in the subject. In some embodiments, the circulating ALP is decreased by about 2.5% or more, about 5% or more, or about 7.5% or more, as compared to prior to administration. In some embodiments, the circulating ALP is decreased by about 10% or more, as compared to prior to administration. In some embodiments, the circulating ALP is decreased by about 20% or more, about 30% or more, about 40% or more, about 50% or more, about 60% or more, about 70% or more, about 80% or more, or about 90% or more, as compared to prior to administration. In some embodiments, the circulating ALP is decreased by no more than about 2.5%, no more than about 5%, or no more than about 7.5%, as compared to prior to administration. In some embodiments, the circulating ALP is decreased by no more than about 10%, as compared to prior to administration. In some embodiments, the circulating ALP is decreased by no more than about 20%, no more than about 30%, no more than about 40%, no more than about 50%, no more than about 60%, no more than about 70%, no more than about 80%, or no more than about 90%, as compared to prior to administration. In some embodiments, the circulating ALP is decreased by 2.5%, 5%, 7.5%, 10%, 15%, 20%, 25%, 30%, 35%, 40%, 45%, 50%, 55%, 60%, 65%, 70%, 75%, 80%, 85%, or 90%, or by a range defined by any of the two aforementioned percentages.

In some embodiments, the composition comprises an oligonucleotide that targets PLIN1 and when administered to a subject in an effective amount decreases circulating gamma-glutamyl transferase (GGT) in the subject. In some embodiments, the circulating GGT is decreased by about 2.5% or more, about 5% or more, or about 7.5% or more, as compared to prior to administration. In some embodiments, the circulating GGT is decreased by about 10% or more, as compared to prior to administration. In some embodiments, the circulating GGT is decreased by about 20% or more, about 30% or more, about 40% or more, about 50% or more, about 60% or more, about 70% or more, about 80% or more, or about 90% or more, as compared to prior to administration. In some embodiments, the circulating GGT is decreased by no more than about 2.5%, no more than about 5%, or no more than about 7.5%, as compared to prior to administration. In some embodiments, the circulating GGT is decreased by no more than about 10%, as compared to prior to administration. In some embodiments, the circulating GGT is decreased by no more than about 20%, no more than about 30%, no more than about 40%, no more than about 50%, no more than about 60%, no more than about 70%, no more than about 80%, or no more than about 90%, as compared to prior to administration. In some embodiments, the circulating GGT is decreased by 2.5%, 5%, 7.5%, 10%, 15%, 20%, 25%, 30%, 35%, 40%, 45%, 50%, 55%, 60%, 65%, 70%, 75%, 80%, 85%, or 90%, or by a range defined by any of the two aforementioned percentages.

In some embodiments, the composition comprises an oligonucleotide that targets PLIN1 and when administered to a subject in an effective amount decreases a nonalcoholic fatty liver disease (NAFLD) fibrosis score in the subject. In some embodiments, the NAFLD fibrosis score is decreased by about 2.5% or more, about 5% or more, or about 7.5% or more, as compared to prior to administration. In some embodiments, the NAFLD fibrosis score is decreased by about 10% or more, as compared to prior to administration. In some embodiments, the NAFLD fibrosis score is decreased by about 20% or more, about 30% or more, about 40% or more, about 50% or more, about 60% or more, about 70% or more, about 80% or more, or about 90% or more, as compared to prior to administration. In some embodiments, the NAFLD fibrosis score is decreased by no more than about 2.5%, no more than about 5%, or no more than about 7.5%, as compared to prior to administration. In some embodiments, the NAFLD fibrosis score is decreased by no more than about 10%, as compared to prior to administration. In some embodiments, the NAFLD fibrosis score is decreased by no more than about 20%, no more than about 30%, no more than about 40%, no more than about 50%, no more than about 60%, no more than about 70%, no more than about 80%, or no more than about 90%, as compared to prior to administration. In some embodiments, the NAFLD fibrosis score is decreased by: 2.5%, 5%, 7.5%, 10%, 15%, 20%, 25%, 30%, 35%, 40%, 45%, 50%, 55%, 60%, 65%, 70%, 75%, 80%, 85%, or 90%, or by a range defined by any of the two aforementioned percentages.

In some embodiments, the composition comprises an oligonucleotide that targets PLIN1 and when administered to a subject in an effective amount decreases a nonalcoholic fatty liver disease (NAFLD) activity score in the subject. In some embodiments, the NAFLD activity score is decreased by about 2.5% or more, about 5% or more, or about 7.5% or more, as compared to prior to administration. In some embodiments, the NAFLD activity score is decreased by about 10% or more, as compared to prior to administration. In some embodiments, the NAFLD activity score is decreased by about 20% or more, about 30% or more, about 40% or more, about 50% or more, about 60% or more, about 70% or more, about 80% or more, or about 90% or more, as compared to prior to administration. In some embodiments, the NAFLD activity score is decreased by no more than about 2.5%, no more than about 5%, or no more than about 7.5%, as compared to prior to administration. In some embodiments, the NAFLD activity score is decreased by no more than about 10%, as compared to prior to administration. In some embodiments, the NAFLD activity score is decreased by no more than about 20%, no more than about 30%, no more than about 40%, no more than about 50%, no more than about 60%, no more than about 70%, no more than about 80%, or no more than about 90%, as compared to prior to administration. In some embodiments, the NAFLD activity score is decreased by 2.5%, 5%, 7.5%, 10%, 15%, 20%, 25%, 30%, 35%, 40%, 45%, 50%, 55%, 60%, 65%, 70%, 75%, 80%, 85%, or 90%, or by a range defined by any of the two aforementioned percentages.

In some embodiments, the composition comprises an oligonucleotide that targets PLIN1 and when administered to a subject in an effective amount decreases a liver fat percentage in the subject. In some embodiments, the liver fat percentage is decreased by about 2.5% or more, about 5% or more, or about 7.5% or more, as compared to prior to administration. In some embodiments, the liver fat percentage is decreased by about 10% or more, as compared to prior to administration. In some embodiments, the liver fat percentage is decreased by about 20% or more about 30% or more, about 40% or more, about 50% or more, about 60% or more, about 70% or more, about 80% or more, or about 90% or more, as compared to prior to administration. In some embodiments, the liver fat percentage is decreased by no more than about 2.5%, no more than about 5%, or no more than about 7.5%, as compared to prior to administration. In some embodiments, the liver fat percentage is decreased by no more than about 10%, as compared to prior to administration. In some embodiments, the liver fat percentage is decreased by no more than about 20%, no more than about 30%, no more than about 40%, no more than about 50%, no more than about 60%, no more than about 70%, no more than about 80%, or no more than about 90%, as compared to prior to administration. In some embodiments, the liver fat percentage is decreased by 2.5%, 5%, 7.5%, 10%, 15%, 20%, 25%, 30%, 35%, 40%, 45%, 50%, 55%, 60%, 65%, 70%, 75%, 80%, 85%, or 90%, or by a range defined by any of the two aforementioned percentages.

In some embodiments, the composition comprises an oligonucleotide that targets PLIN1 and when administered to a subject in an effective amount decreases a liver fibrosis score in the subject. In some embodiments, the liver fibrosis score is decreased by about 2.5% or more, about 5% or more, or about 7.5% or more, as compared to prior to administration. In some embodiments, the liver fibrosis score is decreased by about 10% or more, as compared to prior to administration. In some embodiments, the liver fibrosis score is decreased by about 20% or more, about 30% or more about 40% or more, about 50% or more, about 60% or more, about 70% or more, about 80% or more, or about 90% or more, as compared to prior to administration. In some embodiments, the liver fibrosis score is decreased by no more than about 2.5%, no more than about 5%, or no more than about 7.5%, as compared to prior to administration. In some embodiments, the liver fibrosis score is decreased by no more than about 10%, as compared to prior to administration. In some embodiments, the liver fibrosis score is decreased by no more than about 20%, no more than about 30%, no more than about 40%, no more than about 50%, no more than about 60%, no more than about 70%, no more than about 80%, or no more than about 90%, as compared to prior to administration. In some embodiments, the liver fibrosis score is decreased by 2.5%, 5%, 7.5%, 10%, 15%, 20%, 25%, 30%, 35%, 40%, 45%, 50%, 55%, 60%, 65%, 70%, 75%, 80%, 85%, or 90%, or by a range defined by any of the two aforementioned percentages.

In some embodiments, the composition comprises an oligonucleotide that targets PLIN1 and when administered to a subject in an effective amount increases circulating high density lipoprotein (HDL) in the subject. In some embodiments, the circulating HDL is increased by about 2.5% or more, about 5% or more, or about 7.5% or more, as compared to prior to administration. In some embodiments, the circulating HDL is increased by about 10% or more, as compared to prior to administration. In some embodiments, the circulating HDL is increased by about 20% or more, about 30% or more, about 40% or more, about 50% or more, about 60% or more, about 70% or more, about 80% or more, about 90% or more, or about 100% or more, as compared to prior to administration. In some embodiments, the circulating HDL is increased by about 200% or more, about 300% or more, about 400% or more, about 500% or more, about 600% or more, about 700% or more, about 800% or more, about 900% or more, or about 1000% or more, as compared to prior to administration. In some embodiments, the circulating HDL is increased by no more than about 2.5%, no more than about 5%, or no more than about 7.5%, as compared to prior to administration. In some embodiments, the circulating HDL is increased by no more than about 10%, as compared to prior to administration. In some embodiments, the circulating HDL is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the circulating HDL is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the circulating HDL is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

In some embodiments, the composition comprises an oligonucleotide that targets PLIN1 and when administered to a subject in an effective amount increases circulating apolipoprotein A1 (ApoA1) in the subject. In some embodiments, the circulating ApoA1 is increased by about 2.5% or more, about 5% or more, or about 7.5% or more, as compared to prior to administration. In some embodiments, the circulating ApoA1 is increased by about 10% or more, as compared to prior to administration. In some embodiments, the circulating ApoA1 is increased by about 20% or more, about 30% or more, about 40% or more, about 50% or more, about 60% or more, about 70% or more, about 80% or more, about 90% or more, or about 100% or more, as compared to prior to administration. In some embodiments, the circulating ApoA1 is increased by about 200% or more, about 300% or more, about 400% or more, about 500% or more, about 600% or more, about 700% or more, about 800% or more, about 900% or more, or about 1000% or more, as compared to prior to administration. In some embodiments, the circulating ApoA1 is increased by no more than about 2.5%, no more than about 5%, or no more than about 7.5%, as compared to prior to administration. In some embodiments, the circulating ApoA1 is increased by no more than about 10%, as compared to prior to administration. In some embodiments, the circulating ApoA1 is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the circulating ApoA1 is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the circulating ApoA1 is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

In some embodiments, the composition comprises an oligonucleotide that targets PLIN1 and when administered to a subject in an effective amount increases insulin sensitivity in the subject. In some embodiments, the insulin sensitivity is increased by about 2.5% or more, about 5% or more, or about 7.5% or more, as compared to prior to administration. In some embodiments, the insulin sensitivity is increased by about 10% or more, as compared to prior to administration. In some embodiments, the insulin sensitivity is increased by about 20% or more, about 30% or more, about 40% or more, about 50% or more, about 60% or more, about 70% or more, about 80% or more, about 90% or more, or about 100% or more, as compared to prior to administration. In some embodiments, the insulin sensitivity is increased by about 200% or more, about 300% or more, about 400% or more, about 500% or more, about 600% or more, about 700% or more, about 800% or more, about 900% or more, or about 1000% or more, as compared to prior to administration. In some embodiments, the insulin sensitivity is increased by no more than about 2.5%, no more than about 5%, or no more than about 7.5%, as compared to prior to administration. In some embodiments, the insulin sensitivity is increased by no more than about 10%, as compared to prior to administration. In some embodiments, the insulin sensitivity is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the insulin sensitivity is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the insulin sensitivity is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

In some embodiments, the composition comprises an oligonucleotide that targets PLIN1 and when administered to a subject in an effective amount increases left ventricular ejection fraction in the subject. In some embodiments, the left ventricular ejection fraction is increased by about 2.5% or more, about 5% or more, or about 7.5% or more, as compared to prior to administration. In some embodiments, the left ventricular ejection fraction is increased by about 10% or more, as compared to prior to administration. In some embodiments, the left ventricular ejection fraction is increased by about 20% or more, about 30% or more, about 40% or more, about 50% or more, about 60% or more, about 70% or more, about 80% or more, about 90% or more, or about 100% or more, as compared to prior to administration. In some embodiments, the left ventricular ejection fraction is increased by about 200% or more, about 300% or more, about 400% or more, about 500% or more, about 600% or more, about 700% or more, about 800% or more, about 900% or more, or about 1000% or more, as compared to prior to administration. In some embodiments, the left ventricular ejection fraction is increased by no more than about 2.5%, no more than about 5%, or no more than about 7.5%, as compared to prior to administration. In some embodiments, the left ventricular ejection fraction is increased by no more than about 10%, as compared to prior to administration. In some embodiments, the left ventricular ejection fraction is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the left ventricular ejection fraction is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the left ventricular ejection fraction is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

### A. SiRNAs

In some embodiments, the composition comprises an oligonucleotide that targets PLIN1, wherein the oligonucleotide comprises a small interfering RNA (siRNA). In some embodiments, the composition comprises an oligonucleotide that targets PLIN1, wherein the oligonucleotide comprises a small interfering RNA (siRNA) comprising a sense strand and an antisense strand.

In some embodiments, the composition comprises an oligonucleotide that inhibits the expression of PLIN1, wherein the oligonucleotide comprises an siRNA comprising a sense strand and an antisense strand, wherein the sense strand is 12-30 nucleosides in length. In some embodiments, the composition comprises a sense strand that is 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. The sense strand may be 14-30 nucleosides in length. In some embodiments, the composition comprises an antisense strand is 12-30 nucleosides in length. In some embodiments, the composition comprises an antisense strand that is 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. The antisense strand may be 14-30 nucleosides in length.

In some embodiments, the composition comprises an oligonucleotide that inhibits the expression of PLIN1, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of a full-length human PLIN1 mRNA sequence such as SEQ ID NO: 6014. In some embodiments, at least one of the sense strand and the antisense strand comprise a nucleoside sequence comprising at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 6014.

In some embodiments, the composition comprises an oligonucleotide that inhibits the expression of PLIN1, wherein the oligonucleotide comprises an siRNA comprising a sense strand and an antisense strand, wherein the sense strand and the antisense strand form a double-stranded RNA duplex. In some embodiments, the first base pair of the double-stranded RNA duplex is an AU base pair.

In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3″ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the composition comprises an oligonucleotide that inhibits the expression of PLIN1, wherein the oligonucleotide comprises an siRNA comprising a sense strand and an antisense strand, wherein the siRNA binds with a 19mer in a human PLIN1 mRNA. In some embodiments, the siRNA binds with a 12mer, a 13mer, a 14mer, a 15mer, a 16mer, a 17mer, a 18mer, a 19mer, a 20mer, a 21 mer, a 22mer, a 23mer, a 24mer, or a 25mer in a human PLIN1 mRNA.

In some embodiments, the composition comprises an oligonucleotide that inhibits the expression of PLIN1, wherein the oligonucleotide comprises an siRNA comprising a sense strand and an antisense strand, wherein the siRNA binds with a 17mer in a non-human primate PLIN1 mRNA. In some embodiments, the siRNA binds with a 12mer, a 13mer, a 14mer, a 15mer, a 16mer, a 17mer, a 18mer, a 19mer, a 20mer, a 21mer, a 22mer, a 23mer, a 24mer, or a 25mer in a non-human primate PLIN1 mRNA.

In some embodiments, the composition comprises an oligonucleotide that inhibits the expression of PLIN1, wherein the oligonucleotide comprises an siRNA comprising a sense strand and an antisense strand, wherein the siRNA binds with a human PLIN1 mRNA and less than or equal to 20 human off-targets, with no more than 2 mismatches in the antisense strand. In some embodiments, the siRNA binds with a human PLIN1 mRNA and less than or equal to 10 human off-targets, with no more than 2 mismatches in the antisense strand. In some embodiments, the siRNA binds with a human PLIN1 mRNA and less than or equal to 30 human off-targets, with no more than 2 mismatches in the antisense strand. In some embodiments, the siRNA binds with a human PLIN1 mRNA and less than or equal to 40 human off-targets, with no more than 2 mismatches in the antisense strand. In some embodiments, the siRNA binds with a human PLIN1 mRNA and less than or equal to 50 human off-targets, with no more than 2 mismatches in the antisense strand. In some embodiments, the siRNA binds with a human PLIN1 mRNA and less than or equal to 10 human off-targets, with no more than 3 mismatches in the antisense strand. In some embodiments, the siRNA binds with a human PLIN1 mRNA and less than or equal to 20 human off-targets, with no more than 3 mismatches in the antisense strand. In some embodiments, the siRNA binds with a human PLIN1 mRNA and less than or equal to 30 human off-targets, with no more than 3 mismatches in the antisense strand. In some embodiments, the siRNA binds with a human PLIN1 mRNA and less than or equal to 40 human off-targets, with no more than 3 mismatches in the antisense strand. In some embodiments, the siRNA binds with a human PLIN1 mRNA and less than or equal to 50 human off-targets, with no more than 3 mismatches in the antisense strand.

In some embodiments, the composition comprises an oligonucleotide that inhibits the expression of PLIN1, wherein the oligonucleotide comprises an siRNA comprising a sense strand and an antisense strand, siRNA binds with a human PLIN1 mRNA target site that does not harbor an SNP, with a minor allele frequency (MAF) greater or equal to 1% (pos, 2-18). In some embodiments, the MAF is greater or equal to about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

In some embodiments, the composition comprises an oligonucleotide that inhibits the expression of PLIN1, wherein the oligonucleotide comprises an siRNA comprising a sense strand and an antisense strand, wherein the sense strand comprises a nucleoside sequence comprising or consisting of the sequence of any one of SEQ ID NOs: 1-2898, or a nucleic acid sequence thereof having 1 or 2 nucleoside substitutions, additions, or deletions. In some embodiments, the sense strand comprises a nucleoside sequence comprising or consisting of the sequence of any one of SEQ ID NOs: 1-2898, or a nucleic acid sequence thereof having 3 or 4 nucleoside substitutions, additions, or deletions. In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the sense strand comprises a nucleoside sequence comprising or consisting of the sequence of any one of SEQ ID NOs: 1-2898, or a nucleic acid sequence thereof having 1 or 2 nucleoside additions at the 3′ end. In some embodiments, the composition comprises an oligonucleotide that inhibits the expression of PLIN1, wherein the oligonucleotide comprises an siRNA comprising a sense strand and an antisense strand, wherein the sense strand comprises a nucleoside sequence comprising or consisting of the sequence of any one of SEQ ID NOs: 1-2898. In some embodiments, the composition comprises an oligonucleotide that inhibits the expression of PLIN1, wherein the oligonucleotide comprises an siRNA comprising a sense strand and an antisense strand, wherein the antisense strand comprises a nucleoside sequence comprising or consisting of the sequence of any one of SEQ ID NOs: 2899-5796, or a nucleic acid sequence thereof having 1 or 2 nucleoside substitutions, additions, or deletions. In some embodiments, the antisense strand sequence comprises a nucleoside sequence comprising or consisting of the sequence of any one of SEQ ID NOs: 2899-5796, or a nucleic acid sequence thereof having 3 or 4 nucleoside substitutions, additions, or deletions. In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3″ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the antisense strand comprises a nucleoside sequence comprising or consisting of the sequence of any one of SEQ ID NOs: 2899-5796, or a nucleic acid sequence thereof having 1 or 2 nucleoside additions at the 3′ end. In some embodiments, the composition comprises an oligonucleotide that inhibits the expression of PLIN1, wherein the oligonucleotide comprises an siRNA comprising a sense strand and an antisense strand, wherein the antisense strand comprises a nucleoside sequence comprising or consisting of the sequence of any one of SEQ ID NOs: 2899-5796.

In some embodiments, the siRNA comprises a sense strand having a sequence in accordance with any of SEQ ID NOs: 1-2898. In some embodiments, the sense strand sequence comprises or consists of sequence at least 75% identical to any one of SEQ ID NOs: 1-2898, at least 80% identical to any one of SEQ ID NOs: 1-2898, at least 85% identical to of any one of SEQ ID NOs: 1-2898, at least 90% identical to any one of SEQ ID NOs: 1-2898, or at least 95% identical to any one of SEQ ID NOs: 1-2898. In some embodiments, the sense strand sequence comprises or consists of the sequence of any one of SEQ ID NOs: 1-2898, or a sense strand sequence thereof having 1, 2, 3, or 4 nucleoside substitutions, additions, or deletions. In some embodiments, the sense strand sequence comprises or consists of the sequence of any one of SEQ ID NOs: 1-2898, or a sense strand sequence thereof having 1 or 2 nucleoside substitutions, additions, or deletions. In some embodiments, the sense strand sequence comprises or consists of a sequence 100% identical to SEQ ID NOs: 1-2898. The sense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The sense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The sense strand may comprise a modification pattern described herein. The sense strand may comprise an overhang. The sense strand may comprise a lipid moiety. The sense strand may comprise a GalNAc moiety. In some embodiments, the siRNA comprises an antisense strand having a sequence in accordance with any of SEQ ID NOs: 2899-5796. In some embodiments, the antisense strand sequence comprises or consists of sequence at least 75% identical to any one of SEQ ID NOs: 2899-5796, at least 80% identical to any one of SEQ ID NOs: 2899-5796, at least 85% identical to of any one of SEQ ID NOs: 5491-10980, at least 90% identical to any one of SEQ ID NOs: 2899-5796, or at least 95% identical to any one of SEQ ID NOs: 2899-5796. In some embodiments, the antisense strand sequence comprises or consists of the sequence of any one of SEQ ID NOs: 2899-5796, or an antisense strand sequence thereof having 1, 2, 3, or 4 nucleoside substitutions, additions, or deletions. In some embodiments, the antisense strand sequence comprises or consists of the sequence of any one of SEQ ID NOs: 2899-5796, or an antisense strand sequence thereof having 1 or 2 nucleoside substitutions, additions, or deletions. In some embodiments, the antisense strand sequence comprises or consists of a sequence 100% identical to SEQ ID NOs: 2899-5796. The antisense strand sequence may include the first 5, 6, 7, 8, 9, 10, 11, 12, 13, 14, 15, 16, 17, 18, or 19 nucleotides (in the 5′ to 3′ direction) of any of the aforementioned sequences. The antisense strand sequence may include the last 5, 6, 7, 8, 9, 10, 11, 12, 13, 14, 15, 16, 17, 18, or 19 nucleotides (in the 5′ to 3′ direction) of any of the aforementioned sequences. The antisense strand may comprise an overhang. The antisense strand may comprise a modification pattern described herein. The antisense strand may comprise a lipid moiety or a GalNAc moiety.

In some embodiments, the siRNA comprises a sense strand or antisense strand having a sequence in accordance with the sense strand or antisense strand sequence of an siRNA of subset A. In some embodiments, the sense strand or antisense strand comprises a sequence at least 75% identical, at least 80% identical, at least 85% identical, at least 90% identical, or at least 95% identical, to a sense strand or antisense strand sequence of subset A. In some embodiments, the sense strand or antisense strand comprises a sequence of a sense or antisense strand of subset A, or a sequence thereof having 3 or 4 nucleoside substitutions, additions, or deletions. In some embodiments, the sense strand or antisense strand comprises a sequence of a sense or antisense strand of subset A, or a sequence thereof having 1 or 2 nucleoside substitutions, additions, or deletions. In some embodiments, the sense strand or antisense sequence comprises or consists of a sequence 100% identical to a sense strand or antisense strand sequence of subset A. In some embodiments, the siRNA is cross-reactive with a non-human primate (NHP) PLIN1 mRNA. The siRNA may include one or more internucleoside linkages and/or one or more nucleoside modifications. The sense strand or antisense strand may comprise any modifications described herein. The sense strand or antisense strand may comprise a lipid moiety or a GalNAc moiety. Subset A contains 145 siRNAs whose base sequences are shown in Table 4.

In some embodiments, the siRNA comprises a sense strand or antisense strand having a sequence in accordance with the sense strand or antisense strand sequence of an siRNA of subset B. In some embodiments, the sense strand or antisense strand comprises a sequence at least 75% identical, at least 80% identical, at least 85% identical, at least 90% identical, or at least 95% identical, to a sense strand or antisense strand sequence of subset B. In some embodiments, the sense strand or antisense strand comprises a sequence of a sense or antisense strand of subset B, or a sequence thereof having 3 or 4 nucleoside substitutions, additions, or deletions. In some embodiments, the sense strand or antisense strand comprises a sequence of a sense or antisense strand of subset B, or a sequence thereof having 1 or 2 nucleoside substitutions, additions, or deletions. In some embodiments, the sense strand or antisense sequence comprises or consists of a sequence 100% identical to a sense strand or antisense strand sequence of subset B. The sense strand or antisense strand may comprise any modifications described herein. The sense strand or antisense strand may comprise a lipid moiety or a GalNAc moiety. Subset B includes 119 siRNAs whose base sequences are shown in Table 5.

In some embodiments, the siRNA comprises a sense strand or antisense strand having a sequence in accordance with the sense strand or antisense strand sequence of an siRNA of subset C. In some embodiments, the sense strand or antisense strand comprises a sequence at least 75% identical, at least 80% identical, at least 85% identical, at least 90% identical, or at least 95% identical, to a sense strand or antisense strand sequence of subset C. In some embodiments, the sense strand or antisense strand comprises a sequence of a sense or antisense strand of subset C, or a sequence thereof having 3 or 4 nucleoside substitutions, additions, or deletions. In some embodiments, the sense strand or antisense strand comprises a sequence of a sense or antisense strand of subset C, or a sequence thereof having 1 or 2 nucleoside substitutions, additions, or deletions. In some embodiments, the sense strand or antisense sequence comprises or consists of a sequence 100% identical to a sense strand or antisense strand sequence of subset C. The sense strand or antisense strand may comprise any modifications described herein. The sense strand or antisense strand may comprise a lipid moiety or a GalNAc moiety. Subset C includes 77 siRNAs whose base sequences are shown in Table 6.

In some embodiments, the siRNA comprises a sense strand or antisense strand having a sequence in accordance with the sense strand or antisense strand sequence of an siRNA of subset D. In some embodiments, the sense strand or antisense strand comprises a sequence at least 75% identical, at least 80% identical, at least 85% identical, at least 90% identical, or at least 95% identical, to a sense strand or antisense strand sequence of subset D. In some embodiments, the sense strand or antisense strand comprises a sequence of a sense or antisense strand of subset D, or a sequence thereof having 3 or 4 nucleoside substitutions, additions, or deletions. In some embodiments, the sense strand or antisense strand comprises a sequence of a sense or antisense strand of subset D, or a sequence thereof having 1 or 2 nucleoside substitutions, additions, or deletions. In some embodiments, the sense strand or antisense sequence comprises or consists of a sequence 100% identical to a sense strand or antisense strand sequence of subset D. The sense strand or antisense strand may comprise any modifications described herein. The sense strand or antisense strand may comprise a lipid moiety or a GalNAc moiety. Subset D includes 62 siRNAs whose base sequences are shown in Table 7.

In some embodiments, the siRNA comprises a sense strand or antisense strand having a sequence in accordance with the sense strand or antisense strand sequence of an siRNA of subset E. In some embodiments, the sense strand or antisense strand comprises a sequence at least 75% identical, at least 80% identical, at least 85% identical, at least 90% identical, or at least 95% identical, to a sense strand or antisense strand sequence of subset E. In some embodiments, the sense strand or antisense strand comprises a sequence of a sense or antisense strand of subset E, or a sequence thereof having 3 or 4 nucleoside substitutions, additions, or deletions. In some embodiments, the sense strand or antisense strand comprises a sequence of a sense or antisense strand of subset E, or a sequence thereof having 1 or 2 nucleoside substitutions, additions, or deletions. In some embodiments, the sense strand or antisense sequence comprises or consists of a sequence 100% identical to a sense strand or antisense strand sequence of subset E. The sense strand or antisense strand may comprise any modifications described herein. The sense strand or antisense strand may comprise a lipid moiety or a GalNAc moiety. Subset E includes 51 siRNAs whose base sequences are shown in Table 8.

In some embodiments, the siRNA comprises a sense strand or antisense strand having a sequence in accordance with the sense strand or antisense strand sequence of an siRNA of subset F. In some embodiments, the sense strand or antisense strand comprises a sequence at least 75% identical, at least 80% identical, at least 85% identical, at least 90% identical, or at least 95% identical, to a sense strand or antisense strand sequence of subset F. In some embodiments, the sense strand or antisense strand comprises a sequence of a sense or antisense strand of subset F, or a sequence thereof having 3 or 4 nucleoside substitutions, additions, or deletions. In some embodiments, the sense strand or antisense strand comprises a sequence of a sense or antisense strand of subset F, or a sequence thereof having 1 or 2 nucleoside substitutions, additions, or deletions. In some embodiments, the sense strand or antisense sequence comprises or consists of a sequence 100% identical to a sense strand or antisense strand sequence of subset F. The sense strand or antisense strand may comprise any modifications described herein. The sense strand or antisense strand may comprise a lipid moiety or a GalNAc moiety. Subset F includes 49 siRNAs. The siRNAs in subset F include siRNAs from subset A, and are included in Table 9.

In some embodiments, the siRNA comprises the sense strand and/or the antisense strand sequence of an siRNA in any one of Tables 4-9, or a nucleic acid sequence thereof having 3 or 4 nucleoside substitutions, additions, or deletions. In some embodiments, the siRNA comprises the sense strand and/or the antisense strand sequence of an siRNA in any one of Tables 4-9, or a nucleic acid sequence thereof having 1 or 2 nucleoside substitutions, additions, or deletions. In some embodiments, the siRNA comprises the sense strand and/or the antisense strand sequence of an siRNA in any one of Tables 4-9. In some embodiments, the siRNA is cross-reactive with a non-human primate (NHP) PLIN1 mRNA. The siRNA may include one or more internucleoside linkages and/or one or more nucleoside modifications.

In some embodiments, the siRNA comprises a sense strand or antisense strand having a sequence in accordance with the sense strand or antisense strand sequence of an siRNA in Table 22. In some embodiments, the sense strand or antisense strand comprises a sequence at least 75% identical, at least 80% identical, at least 85% identical, at least 90% identical, or at least 95% identical, to a sense strand or antisense strand sequence in Table 22. In some embodiments, the sense strand or antisense strand comprises a sequence of a sense or antisense strand in Table 22, or a sequence thereof having 3 or 4 nucleoside substitutions, additions, or deletions. In some embodiments, the sense strand or antisense strand comprises a sequence of a sense or antisense strand in Table 22, or a sequence thereof having 1 or 2 nucleoside substitutions, additions, or deletions. In some embodiments, the sense strand or antisense sequence comprises or consists of a sequence 100% identical to a sense strand or antisense strand sequence in Table 22. The sense strand or antisense strand may comprise an overhang. The sense strand or antisense strand may comprise any modifications described herein. The sense strand or antisense strand may comprise a lipid moiety or a GalNAc moiety.

In some embodiments, the siRNA comprises a sense strand having a sequence in accordance with SEQ ID NO: 5984. In some embodiments, the sense strand sequence comprises or consists of sequence at least 75% identical to SEQ ID NO: 5984, at least 80% identical to SEQ ID NO: 5984, at least 85% identical to SEQ ID NO: 5984, at least 90% identical to SEQ ID NO: 5984, or at least 95% identical to SEQ ID NO: 5984. In some embodiments, the sense strand sequence comprises or consists of the sequence of SEQ ID NO 5984, or a sense strand sequence thereof having 1, 2, 3, or 4 nucleoside substitutions, additions, or deletions. In some embodiments, the sense strand sequence comprises or consists of the sequence of SEQ ID NO: 5984, or a sense strand sequence thereof having 1 or 2 nucleoside substitutions, additions, or deletions. In some embodiments, the sense strand sequence comprises or consists of a sequence 100% identical to SEQ ID NO: 5984. The sense strand may comprise any modifications or modification pattern described herein. The sense strand may comprise a moiety such as a GalNAc moiety or a lipid moiety. In some embodiments, the siRNA comprises an antisense strand having a sequence in accordance with SEQ ID NO: 6008. In some embodiments, the antisense strand sequence comprises or consists of sequence at least 75% identical to SEQ ID NO: 6008, at least 80% identical to SEQ ID NO: 6008, at least 85% identical to SEQ ID NO: 6008, at least 90% identical to SEQ ID NO: 6008, or at least 95% identical to SEQ ID NO: 6008. In some embodiments, the antisense strand sequence comprises or consists of the sequence of SEQ ID NO 6008, or an antisense strand sequence thereof having 1, 2, 3, or 4 nucleoside substitutions, additions, or deletions. In some embodiments, the antisense strand sequence comprises or consists of the sequence of SEQ ID NO: 6008, or an antisense strand sequence thereof having 1 or 2 nucleoside substitutions, additions, or deletions. In some embodiments, the antisense strand sequence comprises or consists of a sequence 100% identical to SEQ ID NO: 6008. The antisense strand may comprise any modifications or modification pattern described herein. The antisense strand may comprise a moiety such as a GalNAc moiety or a lipid moiety.

In some embodiments, the siRNA comprises a sense strand having a sequence in accordance with SEQ ID NO: 5987. In some embodiments, the sense strand sequence comprises or consists of sequence at least 75% identical to SEQ ID NO: 5987, at least 80% identical to SEQ ID NO: 5987, at least 85% identical to SEQ ID NO: 5987, at least 90% identical to SEQ ID NO: 5987, or at least 95% identical to SEQ ID NO: 5987. In some embodiments, the sense strand sequence comprises or consists of the sequence of SEQ ID NO 5987, or a sense strand sequence thereof having 1, 2, 3, or 4 nucleoside substitutions, additions, or deletions. In some embodiments, the sense strand sequence comprises or consists of the sequence of SEQ ID NO: 5987, or a sense strand sequence thereof having 1 or 2 nucleoside substitutions, additions, or deletions. In some embodiments, the sense strand sequence comprises or consists of a sequence 100% identical to SEQ ID NO: 5987. The sense strand may comprise any modifications or modification pattern described herein. The sense strand may comprise a moiety such as a GalNAc moiety or a lipid moiety. In some embodiments, the siRNA comprises an antisense strand having a sequence in accordance with SEQ ID NO: 6011. In some embodiments, the antisense strand sequence comprises or consists of sequence at least 75% identical to SEQ ID NO: 6011, at least 80% identical to SEQ ID NO: 6011, at least 85% identical to SEQ ID NO: 6011, at least 90% identical to SEQ ID NO: 6011, or at least 95% identical to SEQ ID NO: 6011. In some embodiments, the antisense strand sequence comprises or consists of the sequence of SEQ ID NO 6011, or an antisense strand sequence thereof having 1, 2, 3, or 4 nucleoside substitutions, additions, or deletions. In some embodiments, the antisense strand sequence comprises or consists of the sequence of SEQ ID NO: 6011, or an antisense strand sequence thereof having 1 or 2 nucleoside substitutions, additions, or deletions. In some embodiments, the antisense strand sequence comprises or consists of a sequence 100% identical to SEQ ID NO: 6011. The antisense strand may comprise any modifications or modification pattern described herein. The antisense strand may comprise a moiety such as a GalNAc moiety or a lipid moiety.

In some embodiments, the siRNA comprises a sense strand having a sequence in accordance with SEQ ID NO: 2124. In some embodiments, the sense strand sequence comprises or consists of sequence at least 75% identical to SEQ ID NO: 2124, at least 80% identical to SEQ ID NO: 2124, at least 85% identical to SEQ ID NO: 2124, at least 90% identical to SEQ ID NO: 2124, or at least 95% identical to SEQ ID NO: 2124. In some embodiments, the sense strand sequence comprises or consists of the sequence of SEQ ID NO 2124, or a sense strand sequence thereof having 1, 2, 3, or 4 nucleoside substitutions, additions, or deletions. In some embodiments, the sense strand sequence comprises or consists of the sequence of SEQ ID NO: 2124, or a sense strand sequence thereof having 1 or 2 nucleoside substitutions, additions, or deletions. In some embodiments, the sense strand sequence comprises or consists of a sequence 100% identical to SEQ ID NO: 2124. The sense strand may comprise any modifications or modification pattern described herein. The sense strand may comprise a moiety such as a GalNAc moiety or a lipid moiety. In some embodiments, the siRNA comprises an antisense strand having a sequence in accordance with SEQ ID NO: 5022. In some embodiments, the antisense strand sequence comprises or consists of sequence at least 75% identical to SEQ ID NO: 5022, at least 80% identical to SEQ ID NO: 5022, at least 85% identical to SEQ ID NO: 5022, at least 90% identical to SEQ ID NO: 5022, or at least 95% identical to SEQ ID NO: 5022. In some embodiments, the antisense strand sequence comprises or consists of the sequence of SEQ ID NO 5022, or an antisense strand sequence thereof having 1, 2, 3, or 4 nucleoside substitutions, additions, or deletions. In some embodiments, the antisense strand sequence comprises or consists of the sequence of SEQ ID NO: 5022, or an antisense strand sequence thereof having 1 or 2 nucleoside substitutions, additions, or deletions. In some embodiments, the antisense strand sequence comprises or consists of a sequence 100% identical to SEQ ID NO: 5022. The antisense strand may comprise any modifications or modification pattern described herein. The antisense strand may comprise a moiety such as a GalNAc moiety or a lipid moiety.

In some embodiments, the siRNA comprises a sense strand having a sequence in accordance with SEQ ID NO: 5988. In some embodiments, the sense strand sequence comprises or consists of sequence at least 75% identical to SEQ ID NO: 5988, at least 80% identical to SEQ ID NO: 5988, at least 85% identical to SEQ ID NO: 5988, at least 90% identical to SEQ ID NO: 5988, or at least 95% identical to SEQ ID NO: 5988. In some embodiments, the sense strand sequence comprises or consists of the sequence of SEQ ID NO 5988, or a sense strand sequence thereof having 1, 2, 3, or 4 nucleoside substitutions, additions, or deletions. In some embodiments, the sense strand sequence comprises or consists of the sequence of SEQ ID NO: 5988, or a sense strand sequence thereof having 1 or 2 nucleoside substitutions, additions, or deletions. In some embodiments, the sense strand sequence comprises or consists of a sequence 100% identical to SEQ ID NO: 5988. The sense strand may comprise any modifications or modification pattern described herein. The sense strand may comprise a moiety such as a GalNAc moiety or a lipid moiety. In some embodiments, the siRNA comprises an antisense strand having a sequence in accordance with SEQ ID NO: 6012. In some embodiments, the antisense strand sequence comprises or consists of sequence at least 75% identical to SEQ ID NO: 6012, at least 80% identical to SEQ ID NO: 6012, at least 85% identical to SEQ ID NO: 6012, at least 90% identical to SEQ ID NO: 6012, or at least 95% identical to SEQ ID NO: 6012. In some embodiments, the antisense strand sequence comprises or consists of the sequence of SEQ ID NO 6012, or an antisense strand sequence thereof having 1, 2, 3, or 4 nucleoside substitutions, additions, or deletions. In some embodiments, the antisense strand sequence comprises or consists of the sequence of SEQ ID NO: 6012, or an antisense strand sequence thereof having 1 or 2 nucleoside substitutions, additions, or deletions. In some embodiments, the antisense strand sequence comprises or consists of a sequence 100% identical to SEQ ID NO: 6012. The antisense strand may comprise any modifications or modification pattern described herein. The antisense strand may comprise a moiety such as a GalNAc moiety or a lipid moiety.

### B. ASOs

In some embodiments, the composition comprises an oligonucleotide that inhibits the expression of PLIN1, wherein the oligonucleotide comprises an antisense oligonucleotide (ASO). In some embodiments, the ASO is 12-30 nucleosides in length. In some embodiments, the ASO is 14-30 nucleosides in length. In some embodiments, the ASO is at least about 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. In some embodiments, the ASO is 15-25 nucleosides in length. In some embodiments, the ASO is 20 nucleosides in length.

In some embodiments, the composition comprises an oligonucleotide that inhibits the expression of PLIN1, wherein the oligonucleotide comprises an ASO about 12-30 nucleosides in length and comprising a nucleoside sequence complementary to about 12-30 contiguous nucleosides of a full-length human PLIN1 mRNA sequence such as SEQ ID NO: 6014; wherein (i) the oligonucleotide comprises a modification comprising a modified nucleoside and/or a modified internucleoside linkage, and/or (ii) the composition comprises a pharmaceutically acceptable carrier. In some embodiments, the ASO comprise a nucleoside sequence complementary to at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 6014.

### C. Modification Patterns

In some embodiments, the composition comprises an oligonucleotide that inhibits the expression of PLIN1, wherein the oligonucleotide comprises a modification comprising a modified nucleoside and/or a modified internucleoside linkage, and/or (ii) the composition comprises a pharmaceutically acceptable carrier. In some embodiments, the oligonucleotide comprises a modification comprising a modified nucleoside and/or a modified internucleoside linkage. In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. A phosphorothioate may include a nonbridging oxygen atom in a phosphate backbone of the oligonucleotide that is replaced by sulfur. Modified internucleoside linkages may be included in siRNAs or ASOs. Benefits of the modified internucleoside linkage may include decreased toxicity or improved pharmacokinetics.

In some embodiments, the composition comprises an oligonucleotide that inhibits the expression of PLIN1, wherein the oligonucleotide comprises a modified internucleoside linkage, wherein the oligonucleotide comprises 1, 2, 3, 4, 5, 6, 7, 8, 9, 10, 11, 12, 13, 14, 15, 16, 17, 18, 19, or 20 modified internucleoside linkages, or a range of modified internucleoside linkages defined by any two of the aforementioned numbers. In some embodiments, the oligonucleotide comprises no more than 18 modified internucleoside linkages. In some embodiments, the oligonucleotide comprises no more than 20 modified internucleoside linkages. In some embodiments, the oligonucleotide comprises 2 or more modified internucleoside linkages, 3 or more modified internucleoside linkages, 4 or more modified internucleoside linkages, 5 or more modified internucleoside linkages, 6 or more modified internucleoside linkages, 7 or more modified internucleoside linkages, 8 or more modified internucleoside linkages, 9 or more modified internucleoside linkages, 10 or more modified internucleoside linkages, 11 or more modified internucleoside linkages, 12 or more modified internucleoside linkages, 13 or more modified internucleoside linkages, 14 or more modified internucleoside linkages, 15 or more modified internucleoside linkages, 16 or more modified internucleoside linkages, 17 or more modified internucleoside linkages, 18 or more modified internucleoside linkages, 19 or more modified internucleoside linkages, or 20 or more modified internucleoside linkages.

In some embodiments, the composition comprises an oligonucleotide that inhibits the expression of PLIN1, wherein the oligonucleotide comprises the modified nucleoside. In some embodiments, the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-fluoro, or 2′-deoxy, or a combination thereof. In some embodiments, the modified nucleoside comprises a LNA. In some embodiments, the modified nucleoside comprises a 2′,4′ constrained ethyl nucleic acid. In some embodiments, the modified nucleoside comprises HLA. In some embodiments, the modified nucleoside comprises CeNA. In some embodiments, the modified nucleoside comprises a 2′-methoxyethyl group. In some embodiments, the modified nucleoside comprises a 2′-O-alkyl group. In some embodiments, the modified nucleoside comprises a 2′-O-allyl group. In some embodiments, the modified nucleoside comprises a 2′-fluoro group. In some embodiments, the modified nucleoside comprises a 2′-deoxy group. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises a 2′-O-methyl nucleoside. In some embodiments, the modified nucleoside comprises a 2′-deoxyfluoro nucleoside. In some embodiments, the modified nucleoside comprises a 2′-O-NMA nucleoside. In some embodiments, the modified nucleoside comprises a 2′-O-DMAEOE nucleoside. In some embodiments, the modified nucleoside comprises a 2′-O-aminopropyl (2′-O-AP) nucleoside. In some embodiments, the modified nucleoside comprises 2′-ara-F. In some embodiments, the modified nucleoside comprises one or more 2′ fluoro modified nucleosides. In some embodiments, the modified nucleoside comprises a 2′ O-alkyl modified nucleoside. Benefits of the modified nucleoside may include decreased toxicity or improved pharmacokinetics.

In some embodiments, the oligonucleotide comprises 1, 2, 3, 4, 5, 6, 7, 8, 9, 10, 11, 12, 13, 14, 15, 16, 17, 18, 19, 20, or 21 modified nucleosides, or a range of nucleosides defined by any two of the aforementioned numbers. In some embodiments, the oligonucleotide comprises no more than 19 modified nucleosides. In some embodiments, the oligonucleotide comprises no more than 21 modified nucleosides. In some embodiments, the oligonucleotide comprises 2 or more modified nucleosides, 3 or more modified nucleosides, 4 or more modified nucleosides, 5 or more modified nucleosides, 6 or more modified nucleosides, 7 or more modified nucleosides, 8 or more modified nucleosides, 9 or more modified nucleosides, 10 or more modified nucleosides, 11 or more modified nucleosides, 12 or more modified nucleosides, 13 or more modified nucleosides, 14 or more modified nucleosides, 15 or more modified nucleosides, 16 or more modified nucleosides, 17 or more modified nucleosides, 18 or more modified nucleosides, 19 or more modified nucleosides, 20 or more modified nucleosides, or 21 or more modified nucleosides.

The oligonucleotide may include purines. Examples of purines include adenine (A) or guanine (G), or modified versions thereof. The oligonucleotide may include pyrimidines. Examples of pyrimidines include cytosine (C), thymine (T), or uracil (U), or modified versions thereof.

In some embodiments, purines of the oligonucleotide comprise 2′ fluoro modified purines. In some embodiments, purines of the oligonucleotide comprise 2′-O-methyl modified purines. In some embodiments, purines of the oligonucleotide comprise a mixture of 2′ fluoro and 2-O-methyl modified purines. In some embodiments, all purines of the oligonucleotide comprise 2′ fluoro modified purines. In some embodiments, all purines of the oligonucleotide comprise 2′-O-methyl modified purines. In some embodiments, all purines of the oligonucleotide comprise a mixture of 2′ fluoro and 2′-O-methyl modified purines. Where 2′-O-methyl modifications are described, it is contemplated that a 2-methyl modification may be included, and vice versa.

In some embodiments, pyrimidines of the oligonucleotide comprise 2′ fluoro modified pyrimidines. In some embodiments, pyrimidines of the oligonucleotide comprise 2′-O-methyl modified pyrimidines. In some embodiments, pyrimidines of the oligonucleotide comprise a mixture of 2′ fluoro and 2′-O-methyl modified pyrimidines. In some embodiments, all pyrimidines of the oligonucleotide comprise 2′ fluoro modified pyrimidines. In some embodiments, all pyrimidines of the oligonucleotide comprise 2-O-methyl modified pyrimidines. In some embodiments, all pyrimidines of the oligonucleotide comprise a mixture of 2′ fluoro and 2-O-methyl modified pyrimidines.

In some embodiments, purines of the oligonucleotide comprise 2′ fluoro modified purines, and pyrimidines of the oligonucleotide comprise a mixture of 2′ fluoro and 2′-O-methyl modified pyrimidines. In some embodiments, purines of the oligonucleotide comprise 2′-O-methyl modified purines, and pyrimidines of the oligonucleotide comprise a mixture of 2′ fluoro and 2′-O-methyl modified pyrimidines. In some embodiments, purines of the oligonucleotide comprise 2′ fluoro modified purines, and pyrimidines of the oligonucleotide comprise 2-O-methyl modified pyrimidines. In some embodiments, purines of the oligonucleotide comprise 2′-O-methyl modified purines, and pyrimidines of the oligonucleotide comprise 2′ fluoro modified pyrimidines. In some embodiments, pyrimidines of the oligonucleotide comprise 2′ fluoro modified pyrimidines, and purines of the oligonucleotide comprise a mixture of 2′ fluoro and 2′-O-methyl modified purines. In some embodiments, pyrimidines of the oligonucleotide comprise 2′-O-methyl modified pyrimidines, and purines of the oligonucleotide comprise a mixture of 2′ fluoro and 2′-O-methyl modified purines. In some embodiments, pyrimidines of the oligonucleotide comprise 2′ fluoro modified pyrimidines, and purines of the oligonucleotide comprise 2′-O-methyl modified purines. In some embodiments, pyrimidines of the oligonucleotide comprise 2′-O-methyl modified pyrimidines, and purines of the oligonucleotide comprise 2′ fluoro modified purines.

In some embodiments, all purines of the oligonucleotide comprise 2′ fluoro modified purines, and all pyrimidines of the oligonucleotide comprise a mixture of 2′ fluoro and 2″-O-methyl modified pyrimidines. In some embodiments, all purines of the oligonucleotide comprise 2″-O-methyl modified purines, and all pyrimidines of the oligonucleotide comprise a mixture of 2′ fluoro and 2′-O-methyl modified pyrimidines. In some embodiments, all purines of the oligonucleotide comprise 2′ fluoro modified purines, and all pyrimidines of the oligonucleotide comprise 2-O-methyl modified pyrimidines. In some embodiments, all purines of the oligonucleotide comprise 2′-O-methyl modified purines, and all pyrimidines of the oligonucleotide comprise 2′ fluoro modified pyrimidines. In some embodiments, all pyrimidines of the oligonucleotide comprise 2′ fluoro modified pyrimidines, and all purines of the oligonucleotide comprise a mixture of 2′ fluoro and 2′-O-methyl modified purines. In some embodiments, all pyrimidines of the oligonucleotide comprise 2′-O-methyl modified pyrimidines, and all purines of the oligonucleotide comprise a mixture of 2′ fluoro and 2′-O-methyl modified purines. In some embodiments, all pyrimidines of the oligonucleotide comprise 2′ fluoro modified pyrimidines, and all purines of the oligonucleotide comprise 2′-O-methyl modified purines. In some embodiments, all pyrimidines of the oligonucleotide comprise 2′-O-methyl modified pyrimidines, and all purines of the oligonucleotide comprise 2′ fluoro modified purines.

In some cases, the oligonucleotide comprises a particular modification pattern. In some embodiments, position 9 counting from the 5′ end of the of a strand of the oligonucleotide may have a 2′F modification. In some embodiments, when position 9 of a strand of the oligonucleotide is a pyrimidine, then all purines in a strand of the oligonucleotide have a 2′OMe modification. In some embodiments, when position 9 is the only pyrimidine between positions 5 and 11 of the sense stand, then position 9 is the only position with a 2′F modification in a strand of the oligonucleotide. In some embodiments, when position 9 and only one other base between positions 5 and 11 of a strand of the oligonucleotide are pyrimidines, then both of these pyrimidines are the only two positions with a 2′F modification in a strand of the oligonucleotide. In some embodiments, when position 9 and only two other bases between positions 5 and 11 of a strand of the oligonucleotide are pyrimidines, and those two other pyrimidines are in adjacent positions so that there would be not three 2′F modifications in a row, then any combination of 2′F modifications can be made that give three 2′F modifications in total. In some embodiments, when there are more than 2 pyrimidines between positions 5 and 11 of a strand of the oligonucleotide, then all combinations of pyrimidines having the 2′F modification are allowed that have three to five 2′F modifications in total, provided that a strand of the oligonucleotide does not have three 2′F modifications in a row. In some cases, a strand of the oligonucleotide of any of the siRNAs comprises a modification pattern which conforms to any or all of these a strand of the oligonucleotide rules.

In some embodiments, when position 9 of a strand of the oligonucleotide is a purine, then all purines in a strand of the oligonucleotide have a 2′OMe modification. In some embodiments, when position 9 is the only purine between positions 5 and 11 of the sense stand, then position 9 is the only position with a 2′F modification in a strand of the oligonucleotide. In some embodiments, when position 9 and only one other base between positions 5 and 11 of a strand of the oligonucleotide are purines, then both of these purines are the only two positions with a 2′F modification in a strand of the oligonucleotide. In some embodiments, when position 9 and only two other bases between positions 5 and 11 of a strand of the oligonucleotide are purines, and those two other purines are in adjacent positions so that there would be not three 2′F modifications in a row, then any combination of 2′F modifications can be made that give three 2′F modifications in total. In some embodiments, when there are more than 2 purines between positions 5 and 11 of a strand of the oligonucleotide, then all combinations of purines having the 2′F modification are allowed that have three to five 2′F modifications in total, provided that a strand of the oligonucleotide does not have three 2′F modifications in a row. In some cases, a strand of the oligonucleotide of any of the siRNAs comprises a modification pattern which conforms to any or all of these a strand of the oligonucleotide rules.

In some cases, position 9 of a strand of the oligonucleotide can be a 2 deoxy. In these cases. 2′F and 2′OMe modifications may occur at the other positions of a strand of the oligonucleotide. In some cases, a strand of the oligonucleotide of any of the siRNAs comprises a modification pattern which conforms to these a strand of the oligonucleotide rules.

In some embodiments, position nine of the sense strand comprises a 2′ fluoro-modified pyrimidine. In some embodiments, all purines of the sense strand comprise 2′-O-methyl modified purines. In some embodiments, 1, 2, 3, 4, or 5 pyrimidines between positions 5 and 11 comprise a 2′ fluoro-modified pyrimidine, provided there are not three 2′ fluoro-modified pyrimidines in a row: In some embodiments, the odd-numbered positions of the antisense strand comprise 2′-O-methyl modified nucleotides. In some embodiments, the even-numbered positions of the antisense strand comprise 2 fluoro-modified nucleotides and unmodified deoxyribonucleotide. In some embodiments, the even-numbered positions of the antisense strand comprise 2 fluoro-modified nucleotides, 2′-O-methyl modified nucleotides and unmodified deoxyribonucleotide. In some embodiments, position nine of the sense strand comprises a 2′ fluoro-modified pyrimidine; all purines of the sense strand comprises 2′-O-methyl modified purines: 1, 2, 3, 4, or 5 pyrimidines between positions 5 and 11 comprise a 2′ fluoro-modified pyrimidine, provided there are not three 2′ fluoro-modified pyrimidines in a row: the odd-numbered positions of the antisense strand comprise 2′-O-methyl modified nucleotides; and the even-numbered positions of the antisense strand comprise 2 fluoro-modified nucleotides and unmodified deoxyribonucleotides.

In some embodiments, position nine of the sense strand comprises a 2′ fluoro-modified purine. In some embodiments, all pyrimidines of the sense strand comprise 2′-O-methyl modified purines. In some embodiments, 1, 2, 3, 4, or 5 purines between positions 5 and 11 comprise a 2′ fluoro-modified purine, provided there are not three 2′ fluoro-modified purine in a row. In some embodiments, the odd-numbered positions of the antisense strand comprise 2-O-methyl modified nucleotides. In some embodiments, the even-numbered positions of the antisense strand comprise 2 fluoro-modified nucleotides and unmodified deoxyribonucleotide. In some embodiments, the even-numbered positions of the antisense strand comprise 2′ fluoro-modified nucleotides, 2-O-methyl modified nucleotides and unmodified deoxyribonucleotide. In some embodiments, position nine of the sense strand comprises a 2′ fluoro-modified purine; all pyrimidine of the sense strand comprises 2′-O-methyl modified pyrimidines: 1, 2, 3, 4, or 5 purines between positions 5 and 11 comprise a 2 fluoro-modified purines, provided there are not three 2′ fluoro-modified purines in a row: the odd-numbered positions of the antisense strand comprise 2′-O-methyl modified nucleotides; and the even-numbered positions of the antisense strand comprise 2 fluoro-modified nucleotides and unmodified deoxyribonucleotides. In some embodiments, there are not three 2′ fluoro-modified purines in a row. In some embodiments, there are not three 2′ fluoro-modified pyrimidines in a row.

In some embodiments, position nine of the sense strand comprises an unmodified deoxyribonucleotide. In some embodiments, positions 5, 7, and 8 of the sense strand comprise 2 fluoro-modified nucleotides. In some embodiments, all pyrimidines in positions 10 to 21 of the sense strand comprise 2′-O-methyl modified pyrimidines and all purines in positions 10 to 21 of the comprise 2′-O-methyl modified purines or 2′ fluoro-modified purines. In some embodiments, the odd-numbered positions of the antisense strand comprise 2-O-methyl modified nucleotides. In some embodiments, the even-numbered positions of the antisense strand comprise 2′ fluoro-modified nucleotides and unmodified deoxyribonucleotides. In some embodiments, the even-numbered positions of the antisense strand comprise 2 fluoro-modified nucleotides, 2′-O-methyl modified nucleotides and unmodified deoxyribonucleotides. In some embodiments, position nine of the sense strand comprises an unmodified deoxyribonucleotide: positions 5, 7, and 8 of the sense strand comprise 2 fluoro-modified nucleotides; all pyrimidines in positions 10 to 21 of the sense strand comprise 2′-O-methyl modified pyrimidines and all purines in positions 10 to 21 of the comprise 2′-O-methyl modified purines or 2 fluoro-modified purines: the odd-numbered positions of the antisense strand comprise 2′-O-methyl modified nucleotides; and the even-numbered positions of the antisense strand comprise 2 fluoro-modified nucleotides and unmodified deoxyribonucleotides.

In some embodiments, position nine of the sense strand comprises an unmodified deoxyribonucleotide. In some embodiments, positions 5, 7, and 8 of the sense strand comprise 2 fluoro-modified nucleotides. In some embodiments, all purines in positions 10 to 21 of the sense strand comprise 2′-O-methyl modified purines and all pyrimidines in positions 10 to 21 of the comprise 2′-O-methyl modified pyrimidines or 2 fluoro-modified pyrimidines. In some embodiments, the odd-numbered positions of the antisense strand comprise 2-O-methyl modified nucleotides. In some embodiments, the even-numbered positions of the antisense strand comprise 2′ fluoro-modified nucleotides and unmodified deoxyribonucleotides. In some embodiments, the even-numbered positions of the antisense strand comprise 2 fluoro-modified nucleotides, 2′-O-methyl modified nucleotides and unmodified deoxyribonucleotides. In some embodiments, position nine of the sense strand comprises an unmodified deoxyribonucleotide: positions 5, 7, and 8 of the sense strand comprise 2 fluoro-modified nucleotides; all purines in positions 10 to 21 of the sense strand comprise 2′-O-methyl modified purines and all pyrimidines in positions 10 to 21 of the comprise 2′-O-methyl modified pyrimidines or 2′ fluoro-modified pyrimidines: the odd-numbered positions of the antisense strand comprise 2′-O-methyl modified nucleotides; and the even-numbered positions of the antisense strand comprise 2 fluoro-modified nucleotides and unmodified deoxyribonucleotide.

In some embodiments, the composition comprises an oligonucleotide that inhibits the expression of PLIN1, wherein the oligonucleotide comprises a moiety attached at a 3′ or 5′ terminus of the oligonucleotide. Examples of moieties include a hydrophobic moiety or a sugar moiety, or a combination thereof. In some embodiments, the oligonucleotide is an siRNA having a sense strand, and the moiety is attached to a 5′ end of the sense strand. In some embodiments, the oligonucleotide is an siRNA having a sense strand, and the moiety is attached to a 3′ end of the sense strand. In some embodiments, the oligonucleotide is an siRNA having an antisense strand, and the moiety is attached to a 5′ end of the antisense strand. In some embodiments, the oligonucleotide is an siRNA having an antisense strand, and the moiety is attached to a 3′ end of the antisense strand. In some embodiments, the oligonucleotide is an ASO, and the moiety is attached to a 5′ end of the ASO. In some embodiments, the oligonucleotide is an ASO, and the moiety is attached to a 3′ end of the ASO.

In some embodiments, the oligonucleotide includes a negatively charged group. The negatively charged group may aid in cell or tissue penetration. The negatively charged group may be attached at a 5′ or 3′ end (e.g, a 5′ end) of the oligonucleotide. This may be referred to as an end group. The end group may be or include a phosphorothioate, phosphorodithioate, vinylphosphonate, methylphosphonate, cyclopropyl phosphonate, or a deoxy-C-malonyl. The end group may include an extra 5′ phosphate such as an extra 5′ phosphate. A combination of end groups may be used.

In some embodiments, the oligonucleotide includes a phosphate mimic. In some embodiments, the phosphate mimic comprises vinyl phosphonate. In some embodiments, the vinyl phosphonate comprises a trans-vinylphosphonate. In some embodiments, the vinyl phosphonate comprises a cis-vinylphosphonate. An example of a nucleotide that includes a vinyl phosphonate is shown below.

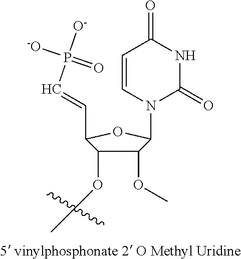

In some embodiments, the vinyl phosphonate increases the stability of the oligonucleotide. In some embodiments, the vinyl phosphonate increases the accumulation of the oligonucleotide in tissues. In some embodiments, the vinyl phosphonate protects the oligonucleotide from an exonuclease or a phosphatase. In some embodiments, the vinyl phosphonate improves the binding affinity of the oligonucleotide with the siRNA processing machinery.

In some embodiments, the oligonucleotide includes 1 vinyl phosphonate. In some embodiments, the oligonucleotide includes 2 vinyl phosphonates. In some embodiments, the oligonucleotide includes 3 vinyl phosphonates. In some embodiments, the oligonucleotide includes 4 vinyl phosphonates. In some embodiments, the antisense strand of the oligonucleotide comprises a vinyl phosphonate at the 5′ end. In some embodiments, the antisense strand of the oligonucleotide comprises a vinyl phosphonate at the 3′ end. In some embodiments, the sense strand of the oligonucleotide comprises a vinyl phosphonate at the 5′ end. In some embodiments, the sense strand of the oligonucleotide comprises a vinyl phosphonate at the 3′ end.

### 1. Hydrophobic Moieties

In some embodiments, the composition comprises an oligonucleotide that inhibits the expression of PLIN1, wherein the oligonucleotide comprises a hydrophobic moiety. The hydrophobic moiety may be attached at a 3′ or 5′ terminus of the oligonucleotide. The hydrophobic moiety may include a lipid such as a fatty acid. The hydrophobic moiety may include a hydrocarbon. The hydrocarbon may be linear. The hydrocarbon may be non-linear. The hydrophobic moiety may include a lipid moiety or a cholesterol moiety, or a combination thereof.

In some embodiments, the composition comprises an oligonucleotide that inhibits the expression of PLIN1, wherein the oligonucleotide comprises a lipid attached at a 3′ or 5′ terminus of the oligonucleotide. An oligonucleotide comprising a hydrophobic moiety may include, or be referred to as a hydrophobic conjugate. Hydrophobic moieties may be useful for enhancing cellular uptake. The hydrophobic moiety may be attached at a 3′ or 5′ terminus of the oligonucleotide. The hydrophobic moiety may include a lipid such as a fatty acid. The hydrophobic moiety may include a hydrocarbon. The hydrocarbon may be linear. The hydrocarbon may be non-linear. The hydrophobic moiety may include a lipid moiety or a cholesterol moiety, or a combination thereof. In some embodiments, the hydrophobic moiety includes a lipid. In some embodiments, the hydrophobic moiety includes a cyclohexanyl. In some embodiments, the hydrophobic moiety is used in a specific format described herein. In some embodiments, the hydrophobic moiety is attached at a 5′ end of a sense strand without any phosphorothioate groups or linkages at the 5′ end.

The hydrophobic moiety may be or include a lipid moiety. In some embodiments, the composition comprises an oligonucleotide that inhibits the expression of PLIN1, wherein the oligonucleotide comprises a lipid attached at a 3′ or 5′ terminus of the oligonucleotide. In some embodiments, the lipid comprises cholesterol, myristyl, palmityl, stearyl, lithocholyl, docosenyl, docosahexaenoyl, myristyl, palmityl stearyl, or α-tocopheryl, or a combination thereof. In some embodiments, the lipid comprises stearoyl, t-butylphenol, n-butylphenyl, octylphenyl, dodecylphenyl, phenyl n-dodecyl, octadecylbenzamide, hexadecylbenzamide, or octadecylcyclohexyl. In some embodiments, the lipid comprises phenyl para C12.

In some embodiments, the oligonucleotide comprises any aspect of the following structure:

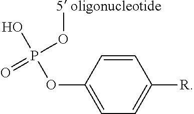

In some embodiments, the oligonucleotide comprises any aspect of the following structure:

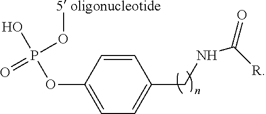

In some embodiments, the oligonucleotide comprises any aspect of the following structure:

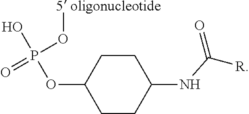

The aspect included in the oligonucleotide may include the entire structure, or may include the lipid moiety, of any of the structures shown. In some embodiments, n is 1-3. In some embodiments, n is 1. In some embodiments, n is 2. In some embodiments, n is 3. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, the alkyl group contains 4-18 carbons. In some embodiments, the lipid moiety comprises an alcohol or ether.

In some embodiments, the lipid includes a fatty acid. In some embodiments, the lipid comprises a lipid depicted in Table 1. The example lipid moieties in Table 1 are shown attached at a 5′ end of an oligonucleotide, in which the 5′ terminal phosphate of the oligonucleotide is shown with the lipid moiety. In some embodiments, a lipid moiety in Table 1 may be attached at a different point of attachment than shown. For example, the point of attachment of any of the lipid moieties in the table may be at a 3′ oligonucleotide end. In some embodiments, the lipid is used for targeting the oligonucleotide to a non-hepatic cell or tissue.

TABLE 1
Lipid moiety examples

| Description | Name | Example depiction attached to an oligonucleotide |
|---|---|---|
| cholesterol-TEG | ETL2 | 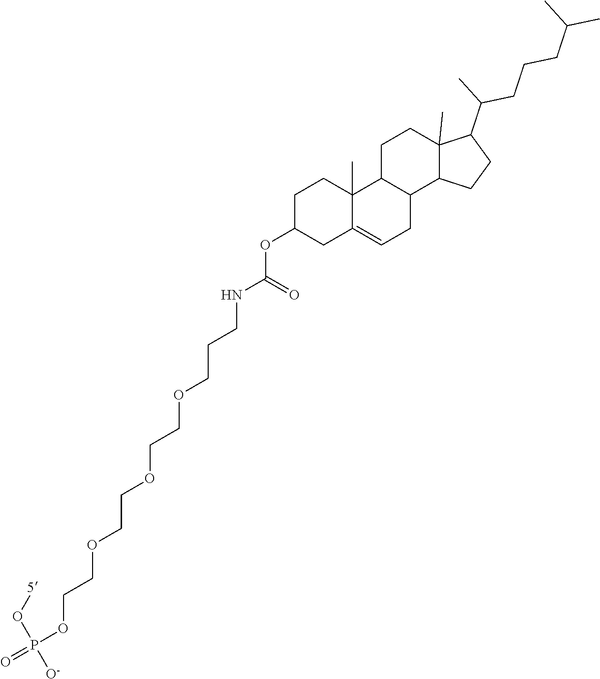 |
| stearyl | ETL3 | 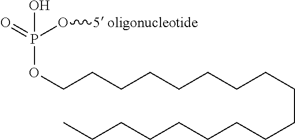 |
| t-butylphenyl | ETL7 | 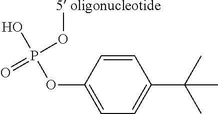 |
| n-butylphenyl | ETL8 | 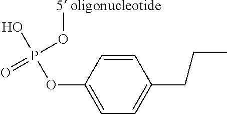 |
| octylphenyl | ETL9 | 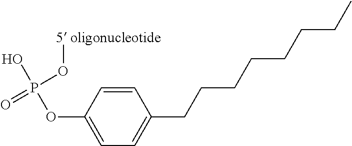 |
| dodecylphenyl | ETL10 | 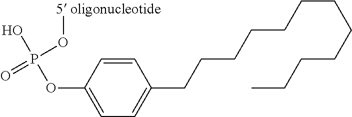 |
| phenyl n-dodecyl | ETL12 | 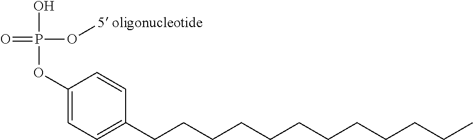 |
| octadecylbenzamide | ETL13 | 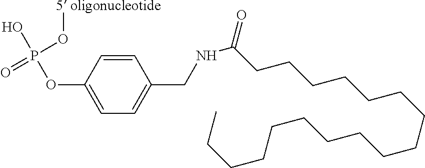 |
| hexadecylbenzamide | ETL15 | 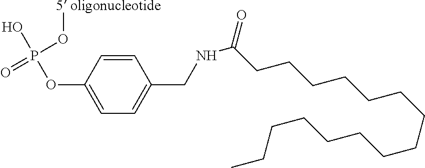 |
| octadecylcyclohexyl | ETL16 | 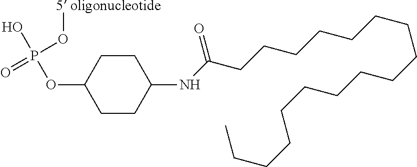 |

In some embodiments, the lipid or lipid moiety includes 16 to 18 carbons. In some embodiments, the lipid includes 16 carbons. In some embodiments, the lipid includes 17 carbons. In some embodiments, the lipid includes 18 carbons. In some embodiments, the lipid moiety includes 16 carbons. In some embodiments, the lipid moiety includes 17 carbons. In some embodiments, the lipid moiety includes 18 carbons.

The hydrophobic moiety may include a linker that comprises a carbocycle. The carbocycle may be six-membered. Some examples of a carbocycle include phenyl or cyclohexyl. The linker may include a phenyl. The linker may include a cyclohexyl. The lipid may be attached to the carbocycle, which may in turn be attached at a phosphate (e.g., 5′ or 3′ phosphate) of the oligonucleotide. In some embodiments, the lipid or hydrocarbon, and the end of the sense are connected to the phenyl or cyclohexyl linker in the 1,4; 1,3; or 1,2 substitution pattern (e.g, the para, meta, or ortho phenyl configuration). In some embodiments, the lipid or hydrocarbon, and the end of the sense are connected to the phenyl or cyclohexyl linker in the 1,4 substitution pattern (e.g, the para phenyl configuration). The lipid may be attached to the carbocycle in the 1,4 substitution pattern relative to the oligonucleotide. The lipid may be attached to the carbocycle in the 1,3 substitution pattern relative to the oligonucleotide. The lipid may be attached to the carbocycle in the 1,2 substitution pattern relative to the oligonucleotide. The lipid may be attached to the carbocycle in the ortho orientation relative to the oligonucleotide. The lipid may be attached to the carbocycle in the para orientation relative to the oligonucleotide. The lipid may be attached to the carbocycle in the meta orientation relative to the oligonucleotide.

The lipid moiety may comprise or consist of the following structure:

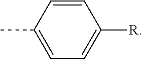

In some embodiments, the lipid moiety comprises or consists of the following structure:

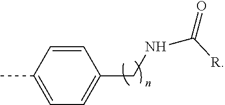

In some embodiments, the lipid moiety comprises or consists of the following structure:

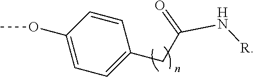

In some embodiments, the lipid moiety comprises or consist of the following structure:

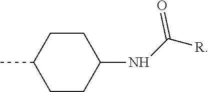

In some embodiments, the dotted line indicates a covalent connection. The covalent connection may between an end of the sense or antisense strand. For example, the connection may be to the 5′ end of the sense strand. In some embodiments, n is 0-3. In some embodiments, n is 1-3. In some embodiments, n is 0. In some embodiments, n is 1. In some embodiments, n is 2. In some embodiments, n is 3. In some embodiments, n is 4. In some embodiments, n is 5. In some embodiments, n is 1, 2, 3, 4, 5, 6, 7, 8, 9, or 10. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, R comprises or consists of an alkyl group containing 4-18 carbons.

The lipid moiety may be attached at a 5′ end of the oligonucleotide. The 5′ end may have one phosphate linking the lipid moiety to a 5′ carbon of a sugar of the oligonucleotide. The 5′ end may have two phosphates linking the lipid moiety to a 5′ carbon of a sugar of the oligonucleotide. The 5′ end may have three phosphates linking the lipid moiety to a 5′ carbon of a sugar of the oligonucleotide. The 5′ end may have one phosphate connected to the 5′ carbon of a sugar of the oligonucleotide, where the one phosphate is connected to the lipid moiety. The 5′ end may have two phosphates connected to the 5′ carbon of a sugar of the oligonucleotide, where the one of the two phosphates is connected to the lipid moiety. The 5′ end may have three phosphates connected to the 5′ carbon of a sugar of the oligonucleotide, where the one of the three phosphates is connected to the lipid moiety. The sugar may include a ribose. The sugar may include a deoxyribose. The sugar may be modified a such as a 2′ modified sugar (e.g, a 2′ O-methyl or 2′ fluoro ribose). A phosphate of the 5′ end may include a modification such as a sulfur in place of an oxygen. Two phosphates of the 5′ end may include a modification such as a sulfur in place of an oxygen. Three phosphates of the 5′ end may include a modification such as a sulfur in place of an oxygen.

In some embodiments, the oligonucleotide includes 1 lipid moiety. In some embodiments, the oligonucleotide includes 2 lipid moieties. In some embodiments, the oligonucleotide includes 3 lipid moieties. In some embodiments, the oligonucleotide includes 4 lipid moieties.

Some embodiments relate to a method of making an oligonucleotide comprising a hydrophobic conjugate. A strategy for making hydrophobic conjugates may include use of a phosphoramidite reagent based upon a 6-membered ring alcohol such as a phenol or cyclohexanol. The phosphoramidite may be reacted to a nucleotide to connect the nucleotide to the hydrophobic moiety, and thereby produce the hydrophobic conjugate. Some examples of phosphoramidite reagents that may be used to produce a hydrophobic conjugate are provided as follows:

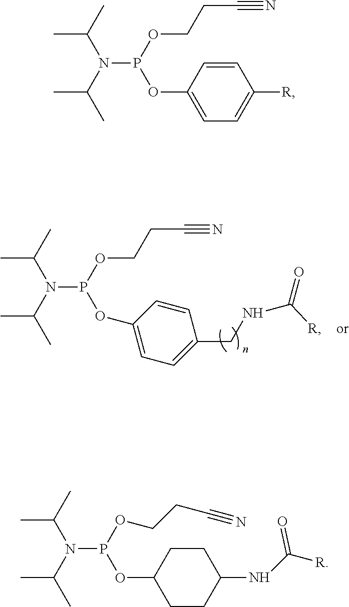

In some embodiments, n is 1-3. In some embodiments, n is 1. In some embodiments, n is 2. In some embodiments, n is 3. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, R comprises or consists of an alkyl group containing 4-18 carbons. Any one of the phosphoramidite reagents may be reacted to a 5′ end of an oligonucleotide to produce an oligonucleotide comprising a hydrophobic moiety. In some embodiments, the phosphoramidite reagents is reacted to a 5′ end of a sense strand of an siRNA. The sense strand may then be hybridized to an antisense strand to form a duplex. The hybridization may be performed by incubating the sense and antisense strands in solution at a given temperature. The temperature may be gradually reduced. The temperature may comprise or include a temperature comprising an annealing temperature for the sense and antisense strands. The temperature may be below or include a temperature below the annealing temperature for the sense and antisense strands. The temperature may be below a melting temperature of the sense and antisense strands.

ETL2 may be conjugated to an oligonucleotide using the following reagent:

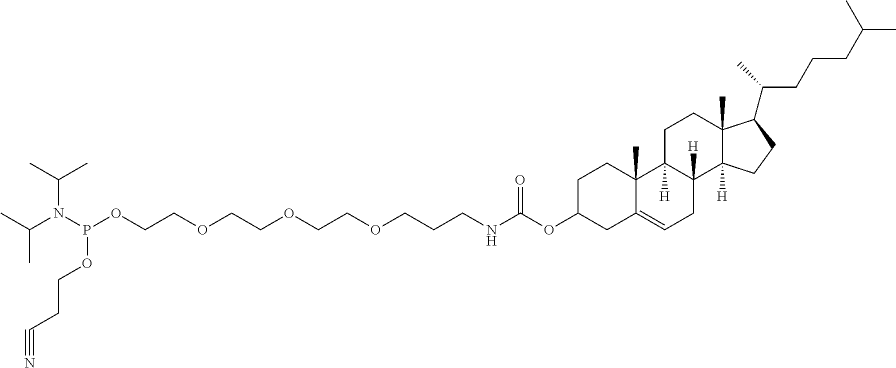

### 2. Sugar Moieties

In some embodiments, the composition comprises an oligonucleotide that inhibits the expression of PLIN1, wherein the oligonucleotide comprises a sugar moiety. The sugar moiety may include an N-acetyl galactose moiety (e.g, an N-acetylgalactosamine (GalNAc) moiety), an N-acetyl glucose moiety (e.g, an N-acetylglucosamine (GlcNAc) moiety), a fucose moiety, or a mannose moiety. The sugar moiety may include 1, 2, 3, or more sugar molecules. The sugar moiety may be attached at a 3′ or 5′ terminus of the oligonucleotide. The sugar moiety may include an N-acetyl galactose moiety. The sugar moiety may include an N-acetylgalactosamine (GalNAc) moiety. The sugar moiety may include an N-acetyl glucose moiety. The sugar moiety may include N-acetylglucosamine (GlcNAc) moiety. The sugar moiety may include a fucose moiety. The sugar moiety may include a mannose moiety. N-acetyl glucose, GlcNAc, fucose, or mannose may be useful for targeting macrophages since they may target or bind a mannose receptor such as CD206. The sugar moiety may be useful for binding or targeting an asialoglycoprotein receptor such as an asialoglycoprotein receptor of a hepatocyte. The GalNAc moiety may bind to an asialoglycoprotein receptor. The GalNAc moiety may target a hepatocyte.

In some embodiments, the composition comprises an oligonucleotide that inhibits the expression of PLIN1, wherein the oligonucleotide comprises an N-acetylgalactosamine (GalNAc) moiety. GalNAc may be useful for hepatocyte targeting. The GalNAc moiety may include 1, 2, 3, or more GalNAc molecules. The GalNAc moiety may be attached at a 3′ or 5′ terminus of the oligonucleotide.

In some embodiments, the composition comprises an oligonucleotide that inhibits the expression of PLIN1, wherein the oligonucleotide comprises an N-acetylgalactosamine (GalNAc) ligand for hepatocyte targeting. In some embodiments, the composition comprises GalNAc. In some embodiments, the composition comprises a GalNAc derivative. In some embodiments, the GalNAc ligand is attached at a 3′ terminus of the oligonucleotide. In some embodiments, the GalNAc ligand is attached at a 5′ terminus of the oligonucleotide. In some embodiments, the composition comprises a sense strand, and the GalNAc ligand is attached to the sense strand (e.g. attached to a 5′ end of the sense strand, or attached to a 3′ end of the sense strand). In some embodiments, the composition comprises an antisense strand, and the GalNAc ligand is attached to the antisense strand (e.g. attached to a 5′ end of the antisense strand, or attached to a 3′ end of the antisense strand). In some embodiments, the composition comprises a GalNAc ligand attached at a 3′ or 5′ terminus of the oligonucleotide.

Disclosed herein, in some embodiments, are compositions comprising an oligonucleotide that inhibits the expression of PLIN1, wherein the oligonucleotide comprises a GalNAc moiety. The GalNAc moiety may be included in any formula, structure, or GalNAc moiety shown below. In some embodiments, described herein is a compound (e.g. oligonucleotide) represented by Formula (I) or (II):

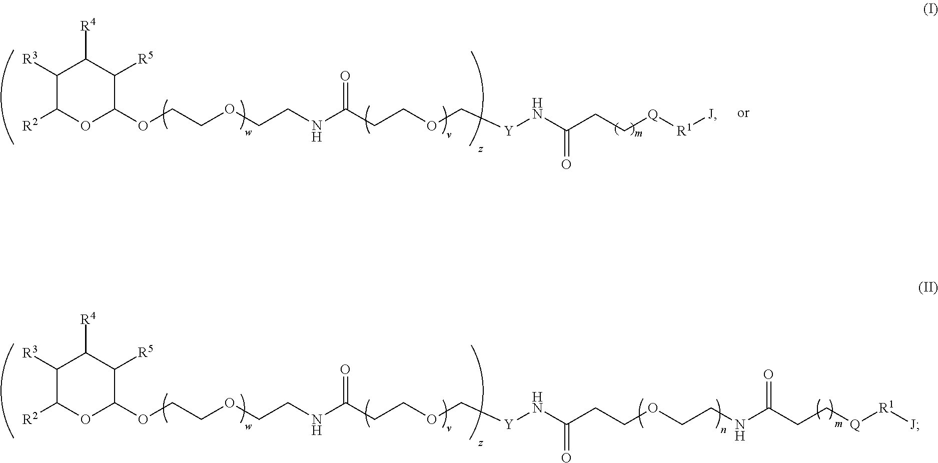

or a salt thereof, wherein
J is an oligonucleotide:
each w is independently selected from any value from 1 to 20;
each v is independently selected from any value from 1 to 20;
n is selected from any value from 1 to 20;
m is selected from any value from 1 to 20;
z is selected from any value from 1 to 3, wherein
   if z is 3, Y is C
   if z is 2, Y is CR⁶, or
   if z is 1, Y is C(R⁶)₂;
Q is selected from:
   C3-10 carbocycle optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, and C1-6 alkyl, wherein the C1-6 alkyl, is optionally substituted with one or more substituents independently selected from halogen, —CN, —OH, —SH, —NO₂, and —NH₂:
R¹ is a linker selected from:
   —O—, —S—, —N(R⁷)—, —C(O)—, —C(O)N(R⁷)—, —N(R⁷)C(O)— —N(R⁷)C(O)N(R⁷)—, —OC(O)N(R⁷)—, —N(R⁷)C(O)O—, —C(O)O—, —OC(O)—, —S(O)—, —S(O)₂—, —OS(O)—, —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻) O—, —OP(S)(O)O—, —OP(O)(S⁻)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂NR⁷—;
each R₂ is independently selected from:
   C₁₋₆ alkyl optionally substituted with one or more substituents independently selected from halogen, —OR⁷, —SR⁷, —N(R⁷), —C(O)R⁷, —C(O)N(R⁷), —N(R⁷)C(O)R⁷, —N(R⁷)C(O)N(R⁷), —OC(O)N(R⁷)₂, —N(R⁷)C(O)OR⁷, —C(O)OR⁷, —OC(O)R⁷, and —S(O)R⁷;
R³ and R⁴ are each independently selected from:
   —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —
   OC(O)N(R⁷)₂, —N(R⁷)C(O)OR⁷, —C(O)OR⁷, —OC(O)R⁷, and —S(O)R⁷:
each R⁵ is independently selected from:
   —OC(O)R⁷, —OC(O)N(R⁷)₂, —N(R⁷)C(O)R⁷—N(R⁷)C(O)N(R⁷)₂, —N(R⁷)C(O)OR⁷, —C(O)R⁷, —C(O)OR⁷, and —C(O)N(R⁷)₂;
each R⁶ is independently selected from:
   hydrogen;
   halogen, —CN, —NO₂, —OR⁷, —SR⁷, —N(R⁷), —C(O)R⁷, —C(O)N(R⁷), —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; and
   C₁₋₆ alkyl optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
each R⁷ is independently selected from:
hydrogen;
C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
C₃₋₁₀ carbocycle, and 3- to 10-membered heterocycle, each of which is optionally substituted with one or more substituents independently selected from halogen, —CN, —OH. —SH, —NO₂, —NH₂, —O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀carbocycle, 3- to 10-membered heterocycle, and C₁₋₆ haloalkyl.

In some embodiments, each w is independently selected from any value from 1 to 10. In some embodiments, each w is independently selected from any value from 1 to 5. In some embodiments, each w is 1. In some embodiments, each v is independently selected from any value from 1 to 10. In some embodiments, each v is independently selected from any value from 1 to 5. In some embodiments, each v is 1. In some embodiments, n is selected from any value from 1 to 10. In some embodiments, n is selected from any value from 1 to 5. In some embodiments, n is 2. In some embodiments, m is selected from any value from 1 to 10. In some embodiments, m is selected from any value from 1 to 5. In some embodiments, m is selected from 1 and 2. In some embodiments, z is 3 and Y is C. In some embodiments, Q is selected from C₅₋₆ carbocycle optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷. In some embodiments, Q is selected from C₅₋₆ carbocycle optionally substituted with one or more substituents independently selected from halogen, —CN, —OH, —SH, —NO₂, and —NH₂. In some embodiments, Q is selected from phenyl and cyclohexyl, each of which is optionally substituted with one or more substituents independently selected from halogen, —CN, —OH, —SH, —NO₂, and —NH₂. In some embodiments, Q is selected from phenyl. In some embodiments, Q is selected from cyclohexyl. In some embodiments, R¹ is selected from—OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, —OP(O)(OR⁷)NR⁷—, —OP(O)(N(R⁷)₂)NR⁷, —OP(OR⁷)O—, —OP(N(R⁷)₂)O—, —OP(OR⁷)N(R⁷)—, and —OPN(R⁷), NR⁷. In some embodiments, R¹ is selected from—OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O)O—, —OP(S)(O⁻)O—, —OP(O)(S⁻)O—, —OP(O)(O⁻)S—, and —OP(OR⁷)O—. In some embodiments, R¹ is selected from—OP(O)(OR⁷)O—, —OP(S)(OR⁷)O—, —OP(O)(O⁻)O—, —OP(S)(O⁻)O—, —OP(O)(S⁻)O—, and —OP(OR⁷)O—. In some embodiments, R¹ is selected from—OP(O)(OR⁷)O— and —OP(OR⁷)O—. In some embodiments, R² is selected from C₁₋₃ alkyl substituted with one or more substituents independently selected from halogen, —OR⁷, —OC(O)R⁷, —SR⁷, —N(R⁷), —C(O)R⁷, and —S(O)R⁷. In some embodiments, R² is selected from C₁₋₃ alkyl substituted with one or more substituents independently selected from—OR⁷, —OC(O)R⁷, —SR⁷, and —N(R⁷)₂. In some embodiments, R² is selected from C₁₋₃ alkyl substituted with one or more substituents independently selected from—OR⁷ and —OC(O)R⁷. In some embodiments, R³ is selected from halogen, —OR⁷, —SR⁷, —N(R⁷)₂, —C(O)R⁷, —OC(O)R⁷, and —S(O)R⁷. In some embodiments, R³ is selected from—OR⁷—SR⁷, —OC(O)R⁷, and —N(R⁷)₂. In some embodiments, R³ is selected from —OR⁷— and —OC(O)R⁷. In some embodiments, R⁴ is selected from halogen, —OR⁷, —SR⁷, —N(R⁷)₂, —C(O)R⁷, —OC(O)R⁷, and —S(O)R⁷. In some embodiments, R⁴ is selected from—OR⁷—SR⁷, —OC(O)R⁷, and —N(R⁷)₂. In some embodiments, R⁴ is selected from—OR⁷— and —OC(O)R⁷. In some embodiments, R⁵ is selected from—OC(O)R⁷, —OC(O)N(R⁷)₂, —N(R⁷)C(O)R⁷—N(R⁷)C(O)N(R⁷)₂, and —N(R⁷)C(O)OR⁷. In some embodiments, R⁵ is selected from—OC(O)R⁷ and —N(R⁷)C(O)R⁷. In some embodiments, each R⁷ is independently selected from: hydrogen; and C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, or 3- to 10-membered heterocycle. In some embodiments, each R⁷ is independently selected from C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl). In some embodiments, each R⁷ is independently selected from C₁₋₆ alkyl optionally substituted with one or more substituents independently selected from halogen, —CN, —OH, and —SH. In some embodiments, w is 1; v is 1; n is 2; m is 1 or 2; z is 3 and Y is C; Q is phenyl or cyclohexyl, each of which is optionally substituted with one or more substituents independently selected from halogen, —CN, —OH, —SH, —NO₂, —NH₂, and C₁₋₃ alkyl; R¹ is selected from—OP(O)(OR⁷)O—, —OP(S)(OR⁷)O—, —OP(O)(O⁻)O—, —OP(S)(O⁻) O—, —OP(O)(S⁻)O—, and —OP(OR⁷)O—; R² is C₁ alkyl substituted with—OH or—OC(O)CH₃; R³ is —OH or—OC(O)CH₃; R⁴ is —OH or —OC(O)CH₃; and R⁵ is —NH(O)CH₃. In some embodiments, the compound comprises:

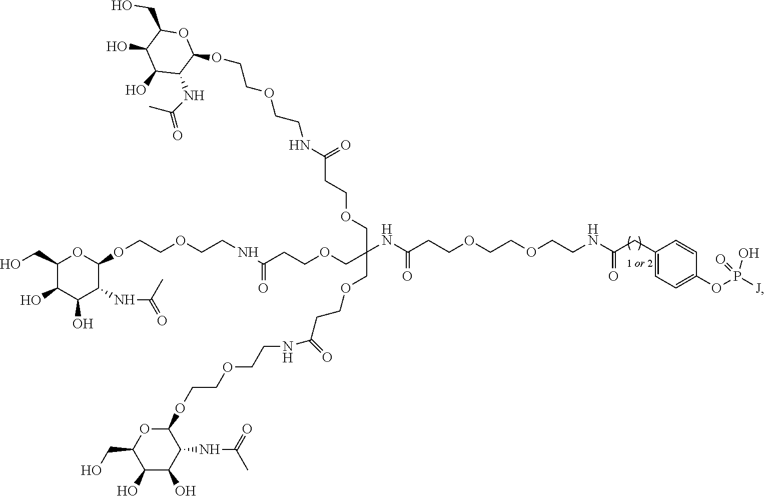

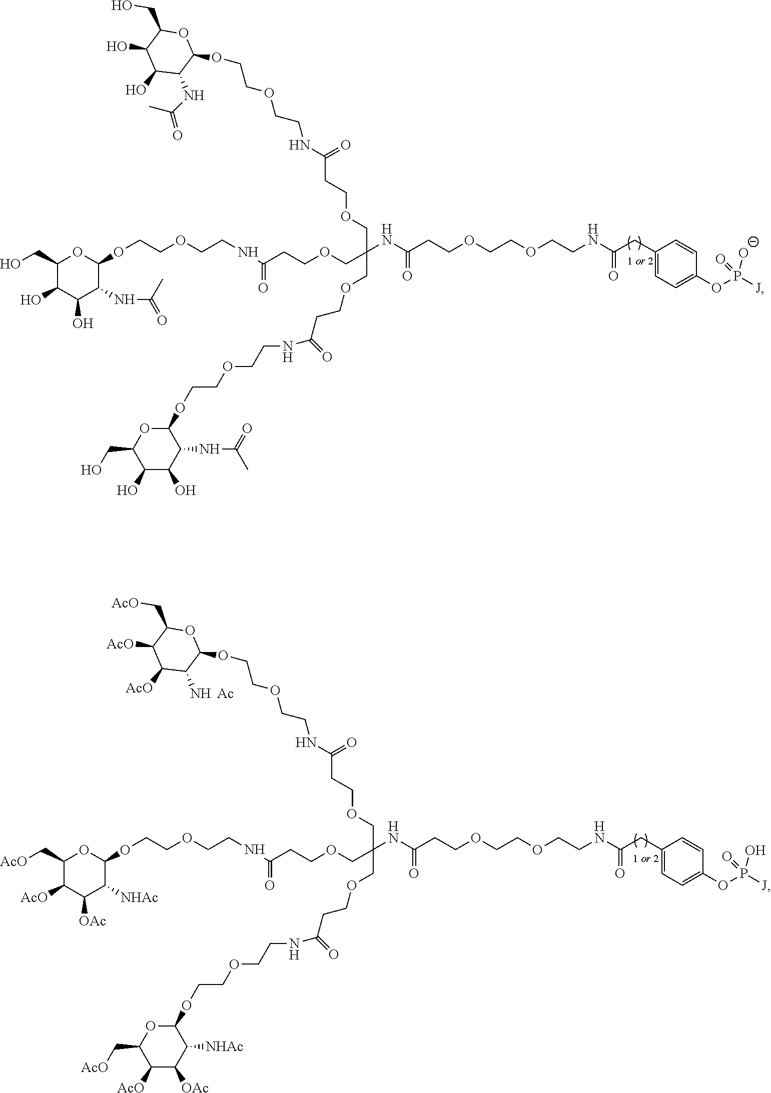

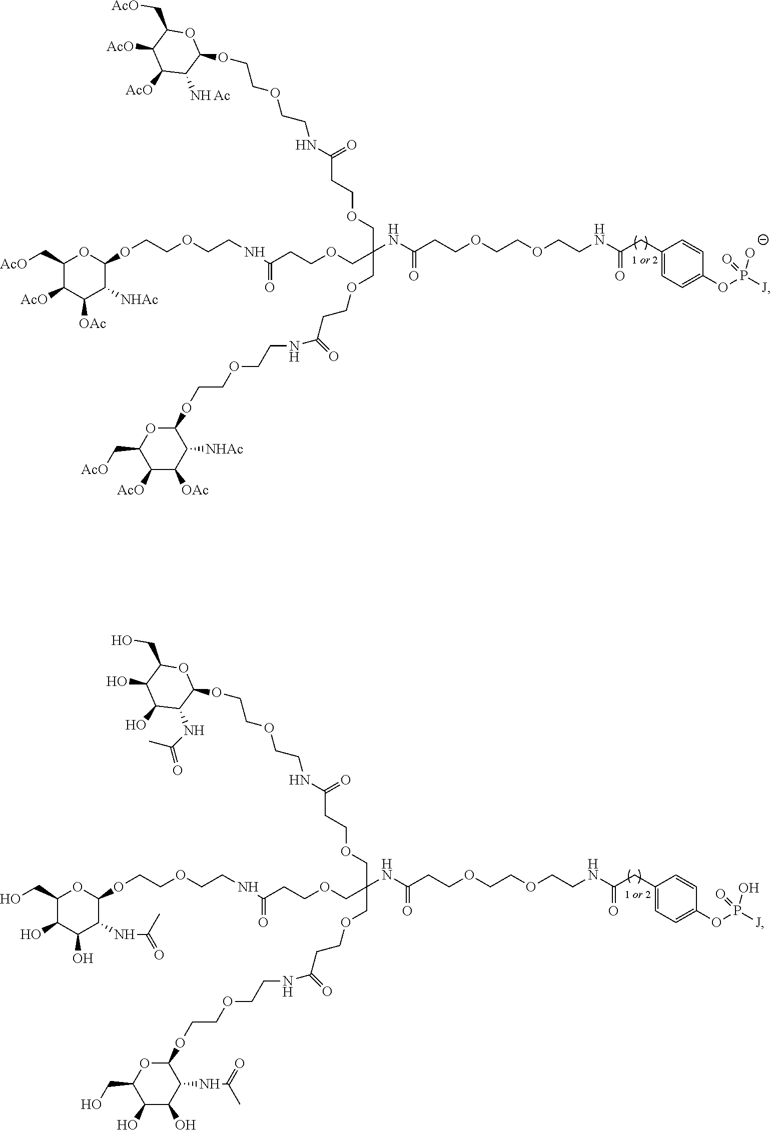

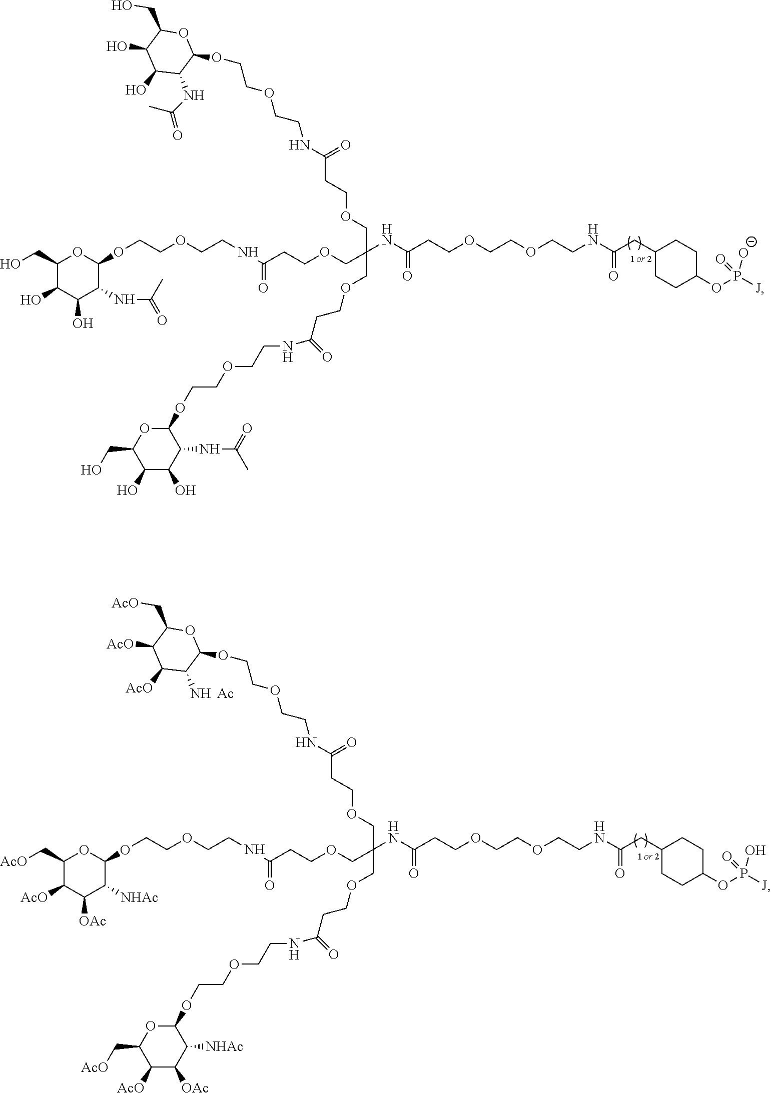

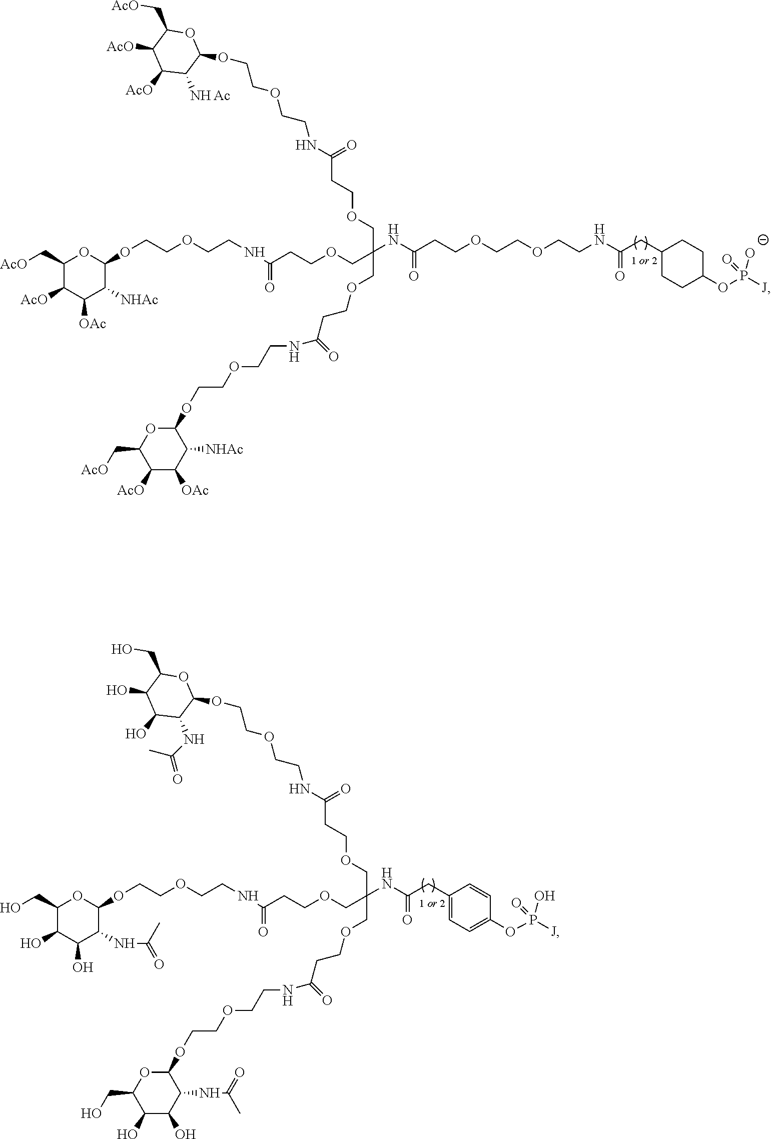

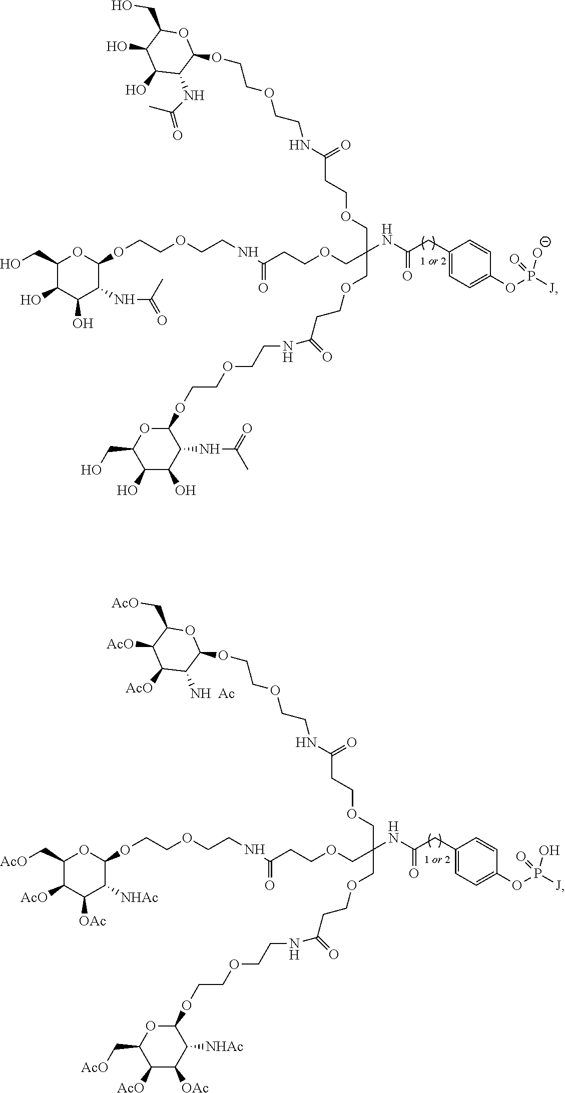

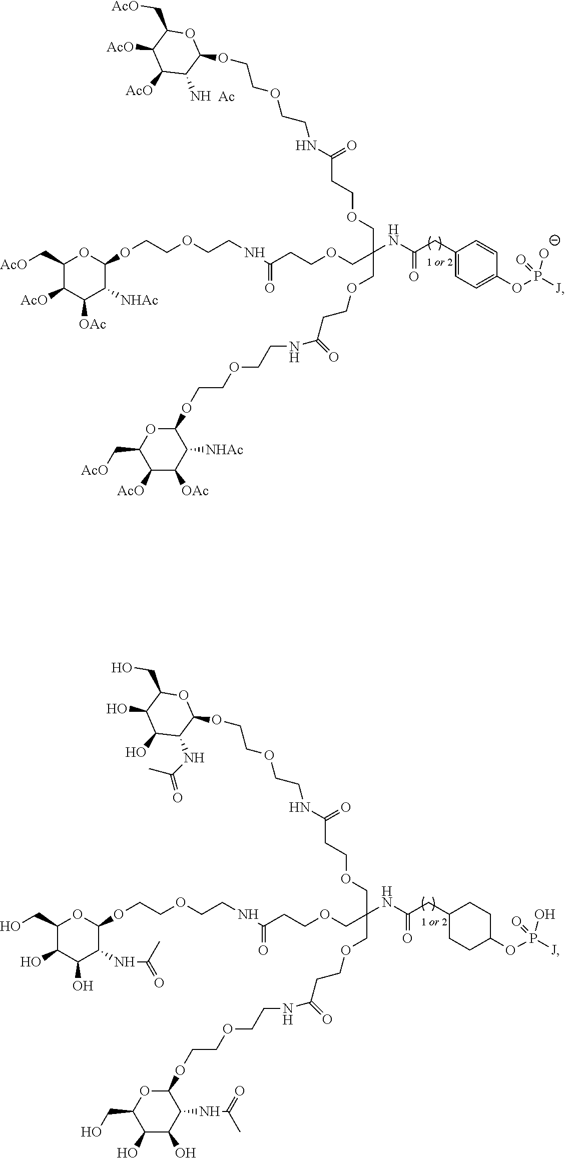

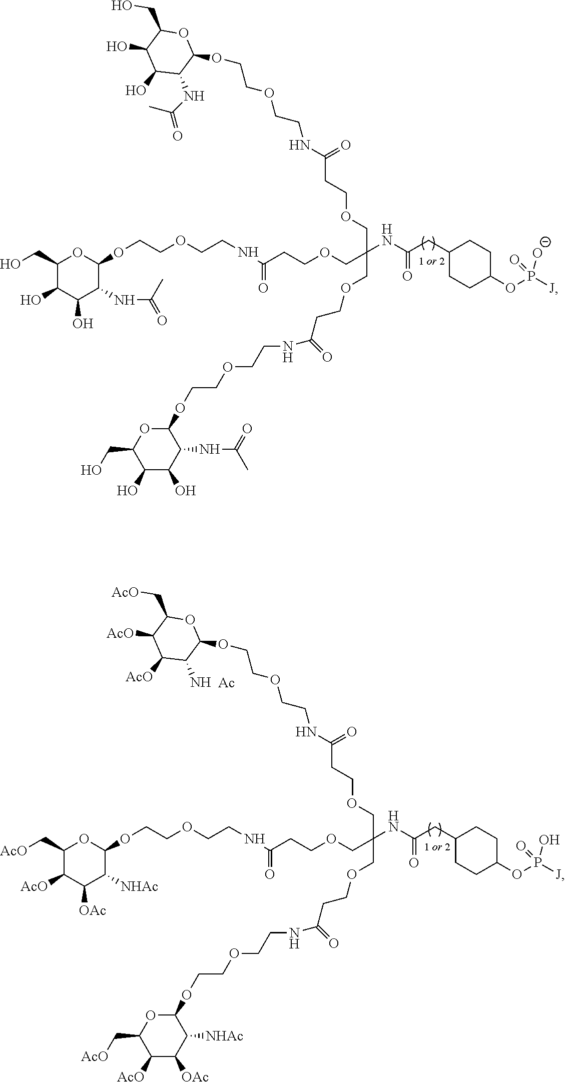

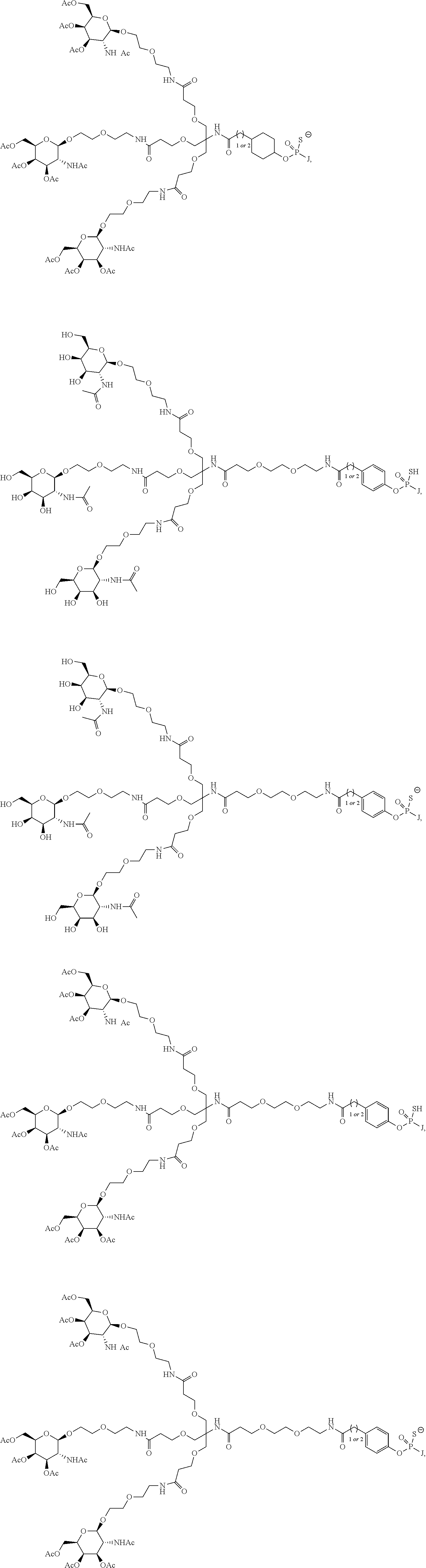

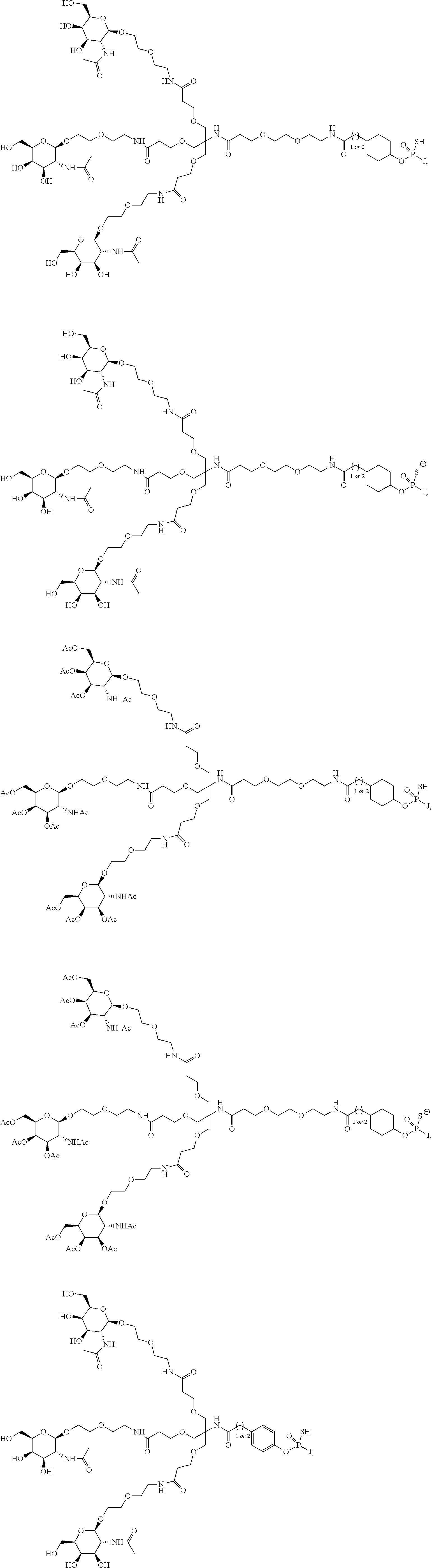

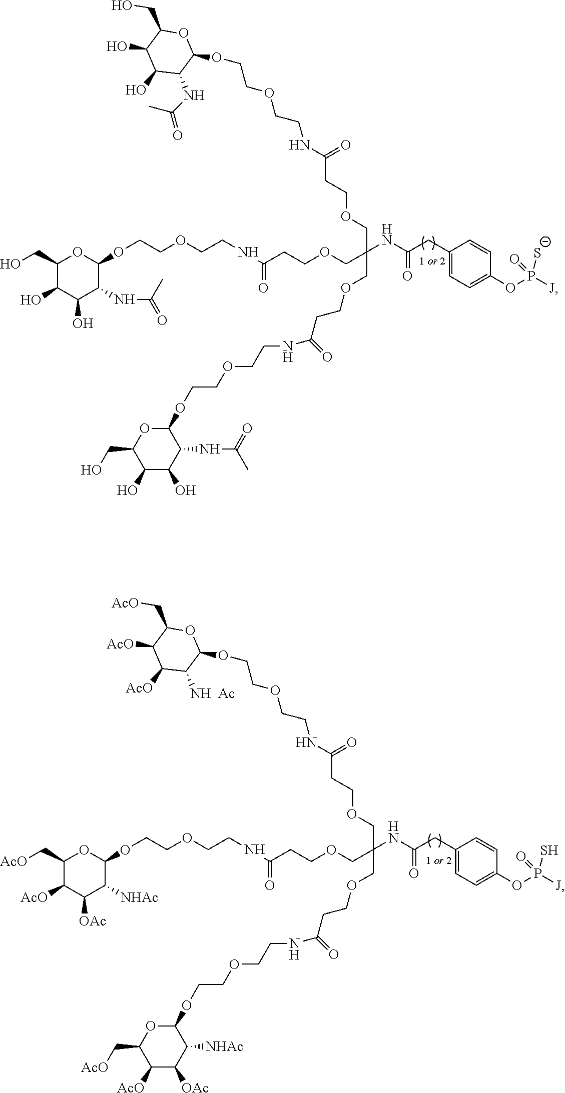

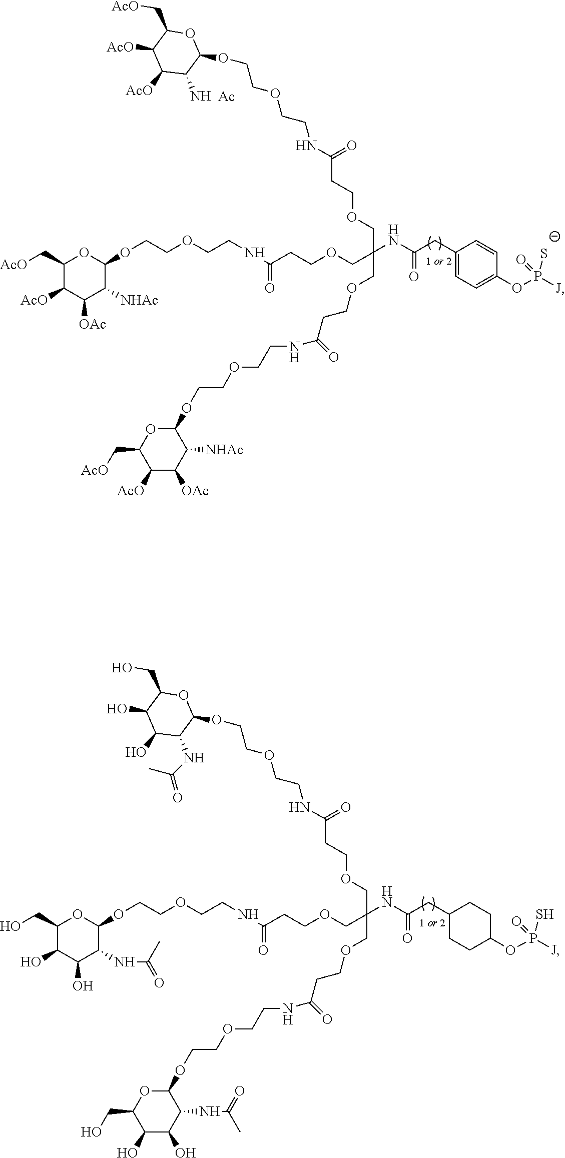

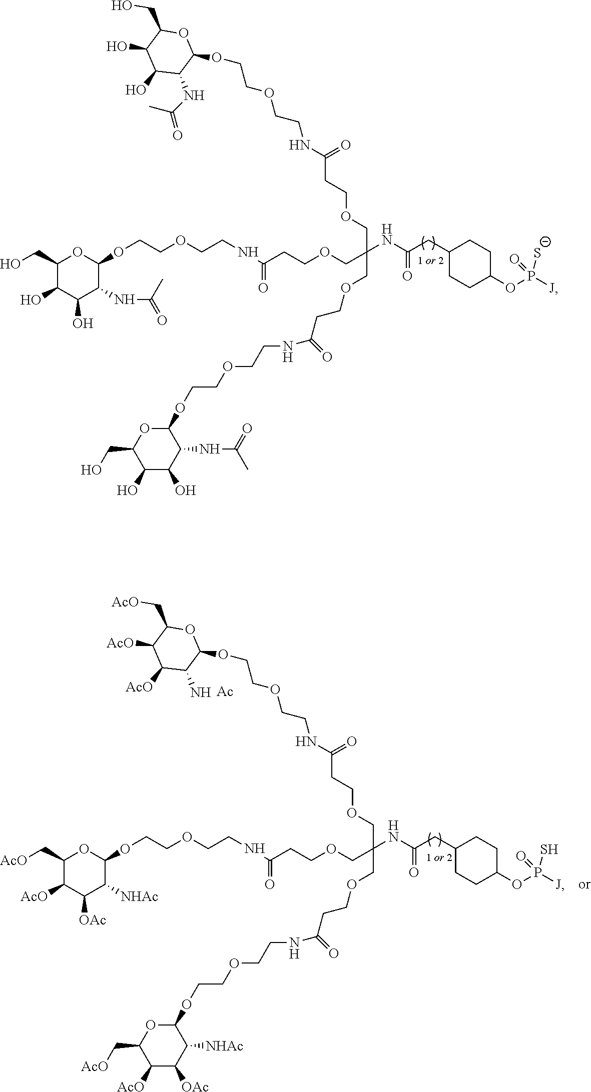

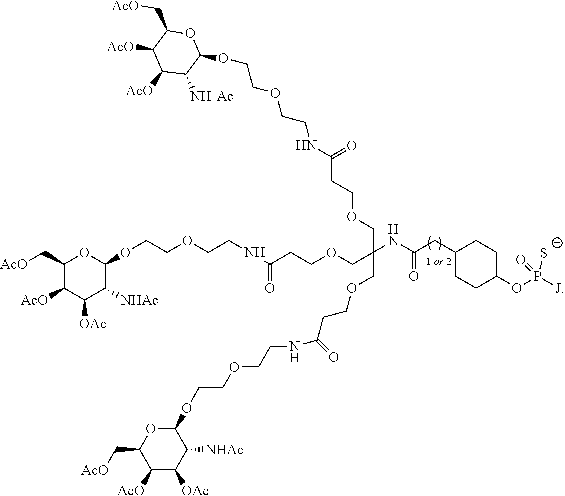

In some embodiments, the oligonucleotide (J) is attached at a 5′ end or a 3′ end of the oligonucleotide. In some embodiments, the oligonucleotide comprises DNA. In some embodiments, the oligonucleotide comprises RNA. In some embodiments, the oligonucleotide comprises one or more modified internucleoside linkages. In some embodiments, the one or more modified internucleoside linkages comprise alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the oligonucleotide comprises 1, 2, 3, 4, 5, 6, 7, 8, 9, 10, 11, 12, 13, 14, 15, 16, 17, 18, 19, or 20 modified internucleoside linkages. In some embodiments, the compound binds to an asialoglycoprotein receptor. In some embodiments, the compound targets a hepatocyte.

Some embodiments include the following, where J is the oligonucleotide:

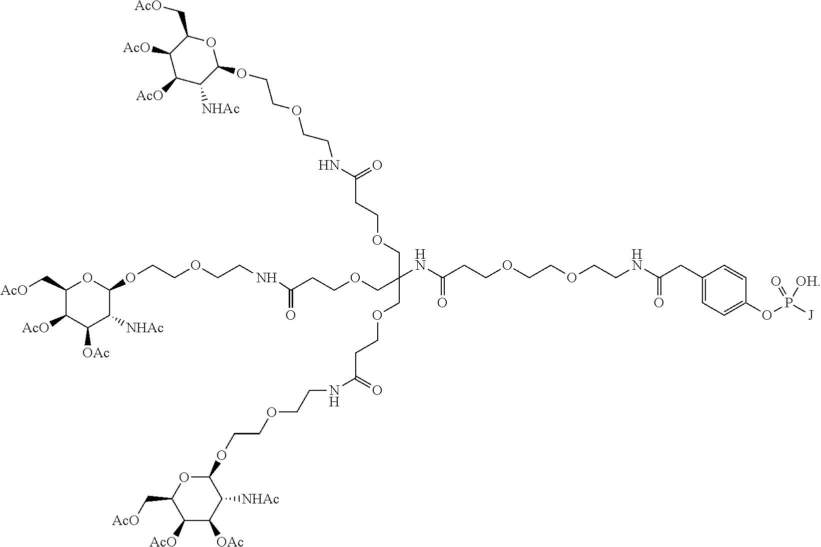

J may include one or more additional phosphates, or one or more phosphorothioates linking to the oligonucleotide. J may include one or more additional phosphates linking to the oligonucleotide. J may include one or more phosphorothioates linking to the oligonucleotide.

Some embodiments include the following, where J is the oligonucleotide:

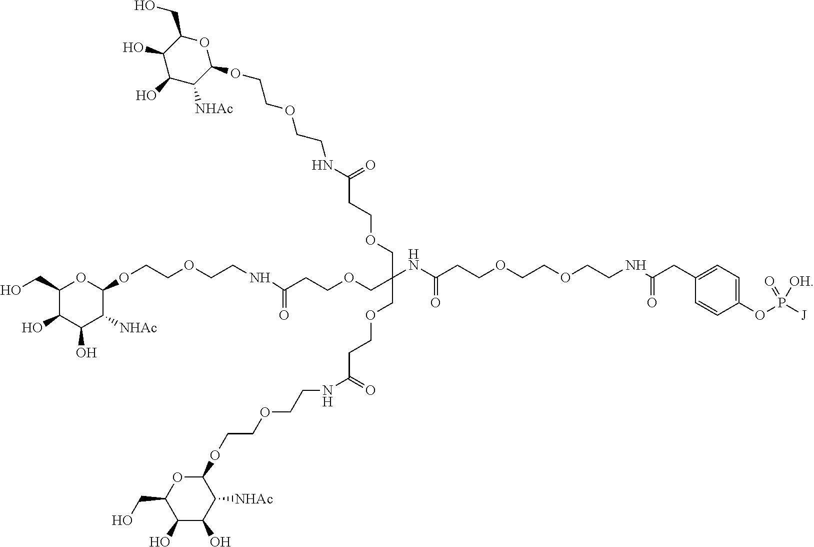

J may include one or more additional phosphates, or one or more phosphorothioates linking to the oligonucleotide. J may include one or more additional phosphates linking to the oligonucleotide. J may include one or more phosphorothioates linking to the oligonucleotide.

Some embodiments include the following, where J is the oligonucleotide:

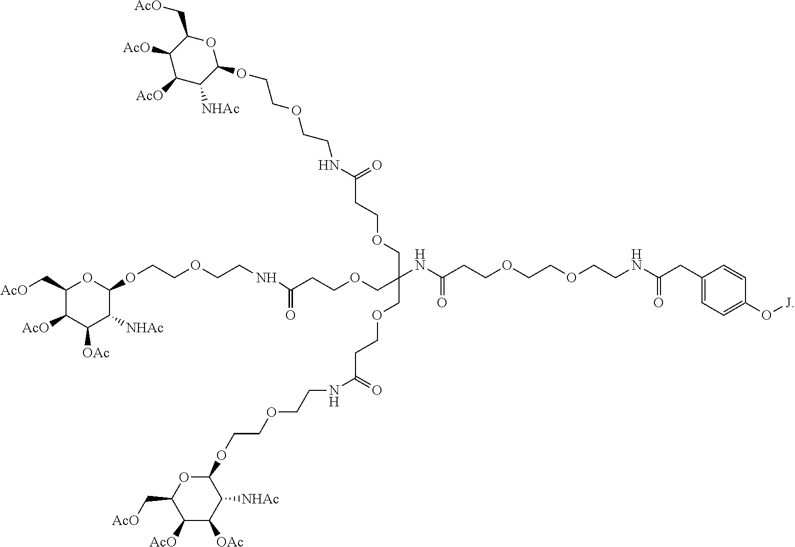

J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where J is the oligonucleotide:

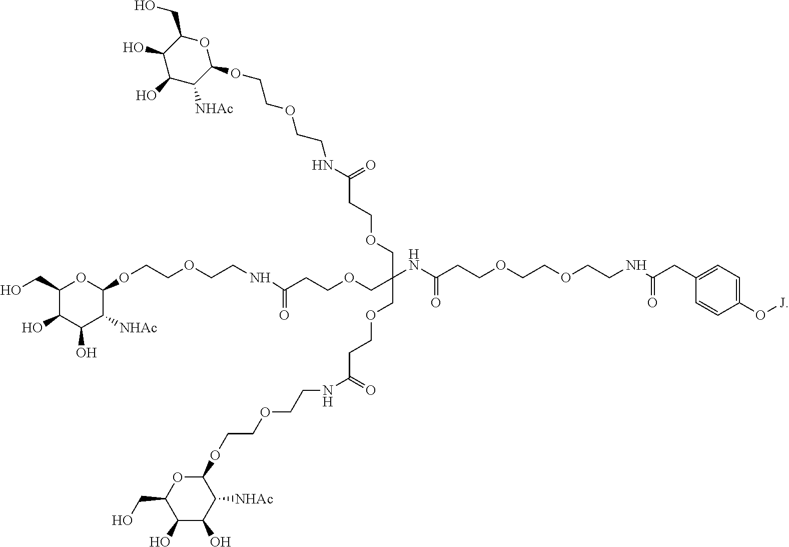

The structure in this compound attached to the oligonucleotide (J) may be referred to as “ETL17,” and is an example of a GalNAc moiety. J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where the phosphate or “5′” indicates a connection to the oligonucleotide:

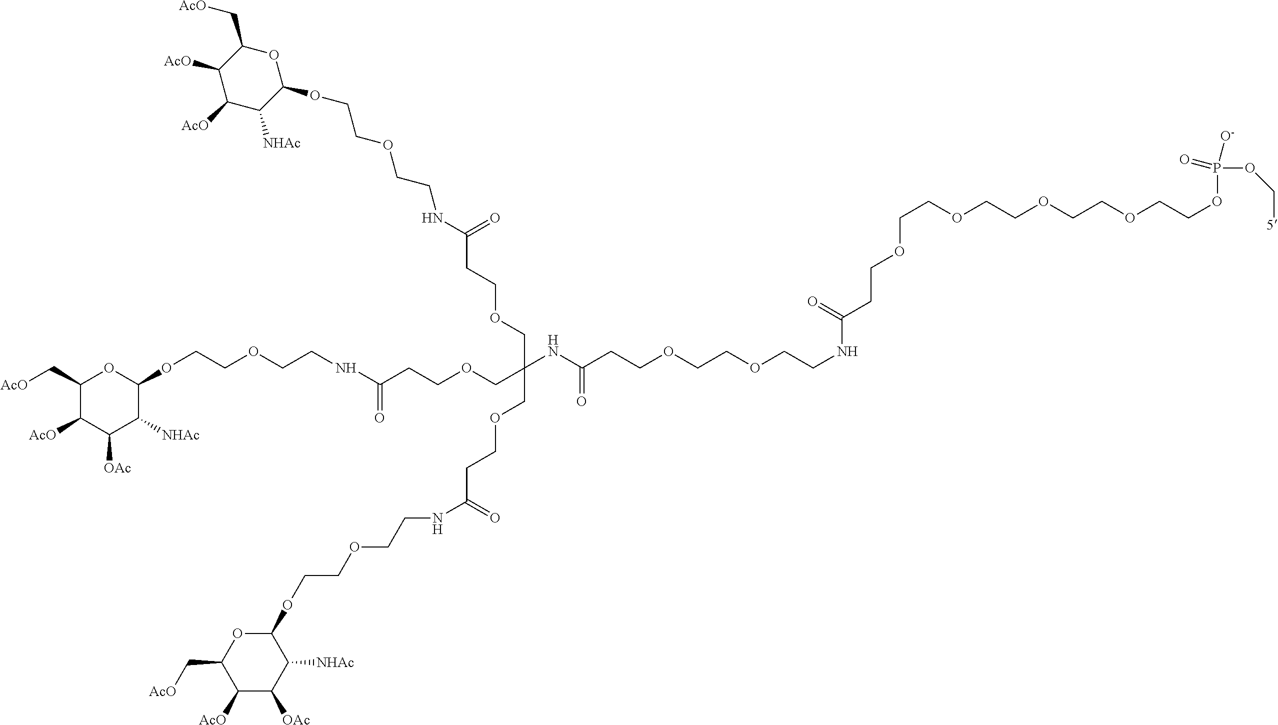

Some embodiments include the following, where the phosphate or “5” indicates a connection to the oligonucleotide:

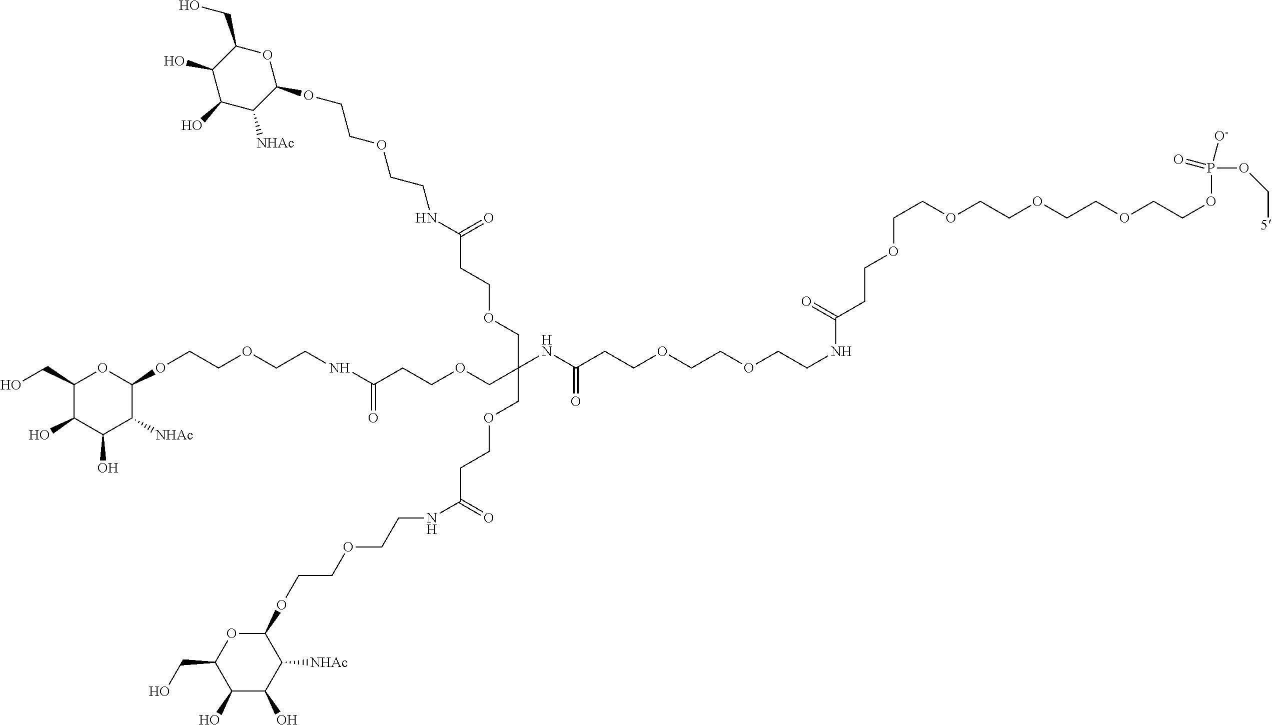

Some embodiments include the following, where J is the oligonucleotide:

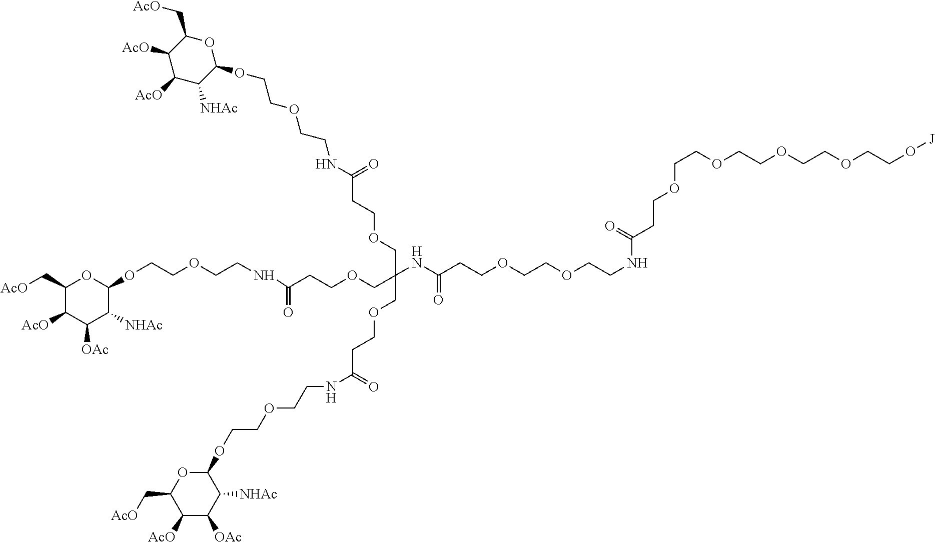

include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where J is the oligonucleotide:

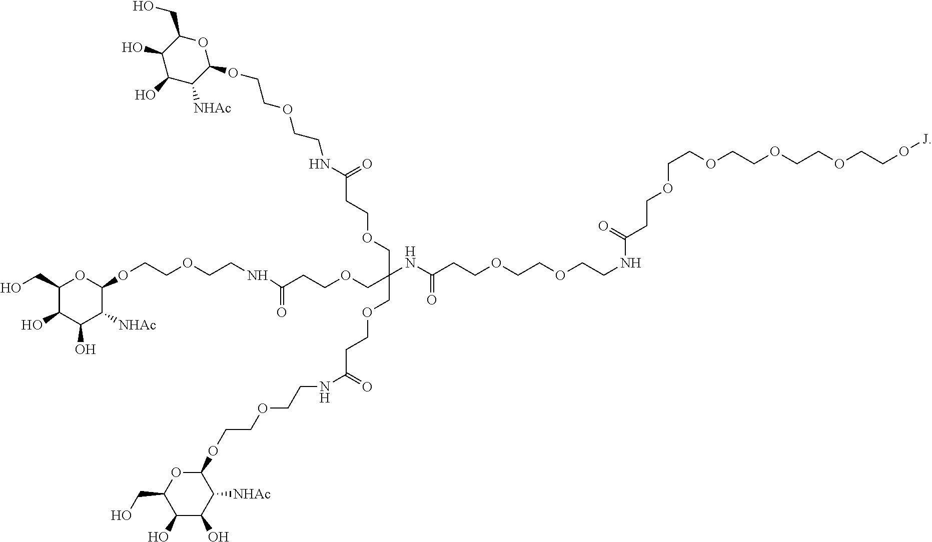

The structure in this compound attached to the oligonucleotide (J) may be referred to as “ETL1,” and is an example of a GalNAc moiety. J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.
3. siRNA Modification Patterns

In some embodiments, the composition comprises an oligonucleotide that inhibits the expression of PLIN1, wherein the oligonucleotide comprises an siRNA comprising a sense strand and an antisense strand, wherein the sense strand comprises modification pattern 1S:
5′-NfsnsNfnNfnNfNfNfnNfnNfnNfnNfnNfsnsn-3′ (SEQ ID NO: 6015), wherein “Nf” is a 2′ fluoro-modified nucleoside. “n” is a 2′ O-methyl modified nucleoside, and “s” is a phosphorothioate linkage. In some embodiments, the sense strand comprises modification pattern 2S:
5′-nsnsnnNfnNfNfNfnnnnnnnnnnsnsn-3′ (SEQ ID NO: 6016), wherein “Nf is a 2” fluoro-modified nucleoside. “n” is a 2′ O-methyl modified nucleoside, and “s” is a phosphorothioate linkage. In some embodiments, the sense strand comprises modification pattern 3S:
5′-nsnsnnNfnNfnNfnnnnnnnnnnsnsn-3″ (SEQ ID NO: 6017), wherein “Nf” is a 2′ fluoro-modified nucleoside. “n” is a 2′ O-methyl modified nucleoside, and “s” is a phosphorothioate linkage. In some embodiments, the sense strand comprises modification pattern 4S:
5′-NfsnsNfnNfnNfNfNfnNfnNfnNfnNfnNfsnsnN-moiety-3″ (SEQ ID NO: 6018), wherein “Nf” is a 2′ fluoro-modified nucleoside. “n” is a 2′ O-methyl modified nucleoside. “s” is a phosphorothioate linkage, and N comprises one or more nucleosides. In some embodiments, the sense strand comprises modification pattern 5S: 5″-nsnsnnNfnNfNfNfnnnnnnnnnnsnsnN-moiety-3″ (SEQ ID NO: 6019), wherein “Nf” is a 2′ fluoro-modified nucleoside. “n” is a 2′ O-methyl modified nucleoside. “s” is a phosphorothioate linkage, and N comprises one or more nucleosides. In some embodiments, the moiety in modification pattern 4S or 5S is a lipid moiety. In some embodiments, the moiety in modification pattern 4S or 5S is a sugar moiety. In some embodiments, the sense strand comprises modification pattern 6S: 5′-NfsnsNfnNfnNfnNfnNfnNfnNfnNfnNfsnsn-3″ (SEQ ID NO: 6020), wherein “Nf” is a 2′ fluoro-modified nucleoside. “n” is a 2′ O-methyl modified nucleoside, and “s” is a phosphorothioate linkage. In some embodiments, the sense strand comprises modification pattern 7S: 5′-nsnsnnNfNfNfNfNfnnnnnnnnnnsnsn-3″ (SEQ ID NO: 6021), wherein “Nf” is a 2′ fluoro-modified nucleoside. “n” is a 2′ O-methyl modified nucleoside, and “s” is a phosphorothioate linkage. In some embodiments, the sense strand comprises modification pattern 8S:
5″-nsnsnnnNfNfNfNfnnnnnnnnnnsnsn-3′ (SEQ ID NO: 6022), wherein “Nf” is a 2′ fluoro-modified nucleoside. “n” is a 2′ O-methyl modified nucleoside, and “s” is a phosphorothioate linkage. In some embodiments, the sense strand comprises modification pattern 9S:
5′-nsnsnnnnNfNfNfNfnnnnnnnnnsnsn-3′ (SEQ ID NO: 6023), wherein “Nf” is a 2′ fluoro-modified nucleoside. “n” is a 2′ O-methyl modified nucleoside, and “s” is a phosphorothioate linkage. In some embodiments, the sense strand comprises modification pattern 10S:
5-NfsnNfnNfnNfNfNfnNfnNfnNfnNfnNfsnsn-3″ (SEQ ID NO: 6024), wherein “Nf” is a 2′ fluoro-modified nucleoside. “n” is a 2′ O-methyl modified nucleoside, and “s” is a phosphorothioate linkage. In some embodiments, the sense strand comprises modification pattern 11S:
5′-NfsnNfnNfnNfnNfnNfnNfnNfnNfnNfsnsn-3″ (SEQ ID NO: 6025), wherein “Nf” is a 2′ fluoro-modified nucleoside. “n” is a 2′ O-methyl modified nucleoside, and “s” is a phosphorothioate linkage. In some embodiments, the sense strand comprises modification pattern 12S:
5-NfnNfnNfnNfnNfnNfnNfnNfnNfnNfsnsn-3′ (SEQ ID NO: 6026), wherein “Nf” is a 2′ fluoro-modified nucleoside. “n” is a 2′ O-methyl modified nucleoside, and “s” is a phosphorothioate linkage. In some embodiments, the sense strand comprises modification pattern 13S:
5′-nnnnnnNfnNfnnnnnnnnnnsnsn-3′ (SEQ ID NO: 6027), wherein “Nf” is a 2′ fluoro-modified nucleoside. “n” is a 2′ O-methyl modified nucleoside, and “s” is a phosphorothioate linkage. In some embodiments, the sense strand comprises modification pattern 14S:
5′-nnnnnnnNfNfNfNfnnnnnnnnsnsn-3′ (SEQ ID NO: 6028), wherein “Nf” is a 2′ fluoro-modified nucleoside. “n” is a 2′ O-methyl modified nucleoside, and “s” is a phosphorothioate linkage. In some embodiments, the sense strand comprises modification pattern 15S:
5′-nnnnNfnnnNfnNfnnnnnnnnsnsn-3″ (SEQ ID NO: 6029), wherein “Nf” is a 2′ fluoro-modified nucleoside. “n” is a 2′ O-methyl modified nucleoside, and “s” is a phosphorothioate linkage. In some embodiments, the sense strand comprises modification pattern 16S:
5′-nnnnnnnNfNfnNfnnnnnnnnsnsn-3″ (SEQ ID NO: 6030), wherein “Nf” is a 2′ fluoro-modified nucleoside. “n” is a 2′ O-methyl modified nucleoside, and “s” is a phosphorothioate linkage. In some embodiments, the sense strand comprises modification pattern 17S:
5′-nnnnnnNfNfNfNfNfnnnnnnnnsnsn-3″ (SEQ ID NO: 6031), wherein “Nf” is a 2′ fluoro-modified nucleoside. “n” is a 2′ O-methyl modified nucleoside, and “s” is a phosphorothioate linkage. In some embodiments, the sense strand comprises modification pattern 18S:
5′-nnnnNfnNfNfNfNfnnnnnnnnnsnsn-3″ (SEQ ID NO: 6032), wherein “Nf is a 2” fluoro-modified nucleoside. “n” is a 2′ O-methyl modified nucleoside, and “s” is a phosphorothioate linkage. In some embodiments, the sense strand comprises modification pattern 19S:
5′-nnnnnNfNfNfNfnNfnnnnnnnnsnsn-3′ (SEQ ID NO: 6033), wherein “Nf” is a 2′ fluoro-modified nucleoside. “n” is a 2′ O-methyl modified nucleoside, and “s” is a phosphorothioate linkage. In some embodiments, the sense strand comprises modification pattern 20S:
5′-nnnnnNfNfNfNfnnnnnnnnnnsnsn-3′ (SEQ ID NO: 6034), wherein “Nf” is a 2′ fluoro-modified nucleoside. “n” is a 2′ O-methyl modified nucleoside, and “s” is a phosphorothioate linkage. In some embodiments, the sense strand comprises modification pattern 21S:
5′-nnnnNfNfNfNfNfnnnnnnnnnnsnsn-3′ (SEQ ID NO: 6035), wherein “Nf” is a 2′ fluoro-modified nucleoside. “n” is a 2′ O-methyl modified nucleoside, and “s” is a phosphorothioate linkage. In some embodiments, the sense strand comprises modification pattern 22S:
5′-nnnnnnnnNfNfnnnnnnnnnsnsn-3′ (SEQ ID NO: 6036), wherein “Nf” is a 2′ fluoro-modified nucleoside. “n” is a 2′ O-methyl modified nucleoside, and “s” is a phosphorothioate linkage. In some embodiments, the sense strand comprises modification pattern 23S:
5′-nnnnnnNfNfNfNfnnnnnnnnnsnsn-3′ (SEQ ID NO: 6037), wherein “Nf” is a 2′ fluoro-modified nucleoside. “n” is a 2′ O-methyl modified nucleoside, and “s” is a phosphorothioate linkage. In some embodiments, the sense strand comprises modification pattern 24S:
5′-nnnnNfNfnnNfnNfnnnnnnnnsnsn-3″ (SEQ ID NO: 6038), wherein “Nf” is a 2′ fluoro-modified nucleoside. “n” is a 2′ O-methyl modified nucleoside, and “s” is a phosphorothioate linkage. In some embodiments, the sense strand comprises modification pattern 25S:
5′-nnnnNfnNfnNfnNfnnnnnnnnsnsn-3′ (SEQ ID NO: 6039), wherein “Nf” is a 2′ fluoro-modified nucleoside. “n” is a 2′ O-methyl modified nucleoside, and “s” is a phosphorothioate linkage. In some embodiments, the sense strand comprises modification pattern 26S:
5′-nnnnnNfnnNfnNfnnnnnnnnsnsn-3′ (SEQ ID NO: 6040), wherein “Nf” is a 2′ fluoro-modified nucleoside. “n” is a 2′ O-methyl modified nucleoside, and “s” is a phosphorothioate linkage. In some embodiments, the sense strand comprises modification pattern 27S:
5′-nnnnnNfNfnNfnnnnnnnnnnsnsn-3′ (SEQ ID NO: 6041), wherein “Nf is a 2” fluoro-modified nucleoside. “n” is a 2′ O-methyl modified nucleoside, and “s” is a phosphorothioate linkage. In some embodiments, the sense strand comprises modification pattern 28S:
5″-nnnnnnNfnNfNfnnnnnnnnnsnsn-3″ (SEQ ID NO: 6042), wherein “Nf” is a 2′ fluoro-modified nucleoside. “n” is a 2′ O-methyl modified nucleoside, and “s” is a phosphorothioate linkage. In some embodiments, the sense strand comprises modification pattern 29S:
5′-nNfnNfnNfnNfNfnnnnnNfnNfNfnsnsn-3′ (SEQ ID NO: 6043), wherein “Nf” is a 2′ fluoro-modified nucleoside. “n” is a 2′ O-methyl modified nucleoside, and “s” is a phosphorothioate linkage. In some embodiments, the sense strand comprises modification pattern 30S:
5′-snnnnnNfNfNfNfnNfnnnnnnnnsnsn-3′ (SEQ ID NO: 6044), wherein “Nf” is a 2′ fluoro-modified nucleoside. “n” is a 2′ O-methyl modified nucleoside, and “s” is a phosphorothioate linkage. In some embodiments, the sense strand comprises modification pattern 31S:
5′-snnnnnNfNfNfNfnnnnnnnnnnsnsn-3′ (SEQ ID NO: 6045), wherein “Nf” is a 2′ fluoro-modified nucleoside. “n” is a 2′ O-methyl modified nucleoside, and “s” is a phosphorothioate linkage. In some embodiments, the sense strand comprises modification pattern 32S:
5″-snnnnNfNfNfNfNfnnnnnnnnnnsnsn-3′ (SEQ ID NO: 6046), wherein “Nf” is a 2′ fluoro-modified nucleoside. “n” is a 2′ O-methyl modified nucleoside, and “s” is a phosphorothioate linkage. In some embodiments, the sense strand comprises modification pattern 33S:
5′-snnnnnnnnNfNfnnnnnnnnnsnsn-3″ (SEQ ID NO: 6047), wherein “Nf” is a 2′ fluoro-modified nucleoside. “n” is a 2′ O-methyl modified nucleoside, and “s” is a phosphorothioate linkage. In some embodiments, the sense strand comprises modification pattern 34S:
5′-snnnnNfNfnnNfnNfnnnnnnnnsnsn-3″ (SEQ ID NO: 6048), wherein “Nf” is a 2′ fluoro-modified nucleoside. “n” is a 2′ O-methyl modified nucleoside, and “s” is a phosphorothioate linkage. In some embodiments, the sense strand comprises modification pattern 35S:
5′-snNfnNfnNfnNfNfnnnnnNfnNfNfnsnsn-3′ (SEQ ID NO: 6049), wherein “Nf” is a 2′ fluoro-modified nucleoside. “n” is a 2′ O-methyl modified nucleoside, and “s” is a phosphorothioate linkage. In some embodiments, the sense strand comprises modification pattern 36S:
5′-nnnnnNfnNfNfnnnnnnnnnnsnsn-3′ (SEQ ID NO: 6050), wherein “Nf is a 2” fluoro-modified nucleoside. “n” is a 2′ O-methyl modified nucleoside, and “s” is a phosphorothioate linkage. In some embodiments, the sense strand comprises modification pattern 37S:
5′-nnnnNfnnNfNfnnnnnnnnnnsnsn-3′ (SEQ ID NO: 6051), wherein “Nf” is a 2′ fluoro-modified nucleoside. “n” is a 2′ O-methyl modified nucleoside, and “s” is a phosphorothioate linkage. In some embodiments, the sense strand comprises modification pattern 38S:
5′-nnnnNfnNfnNfnnnnnnnnnnsnsn-3′ (SEQ ID NO: 6052), wherein “Nf” is a 2′ fluoro-modified nucleoside. “n” is a 2′ O-methyl modified nucleoside, and “s” is a phosphorothioate linkage. In some embodiments, the sense strand comprises modification pattern 39S:
5′-nnnnNfNfnnNfnnnnnnnnnnsnsn-3′ (SEQ ID NO: 6053), wherein “Nf” is a 2′ fluoro-modified nucleoside. “n” is a 2′ O-methyl modified nucleoside, and “s” is a phosphorothioate linkage. In some embodiments, the sense strand comprises modification pattern 40S:
5′-nnnnNfNfnNfNfnnnnnnnnnnsnsn-3′ (SEQ ID NO: 6054), wherein “Nf” is a 2′ fluoro-modified nucleoside. “n” is a 2′ O-methyl modified nucleoside, and “s” is a phosphorothioate linkage. In some embodiments, the sense strand comprises modification pattern 41:
5′-snnnnNfNfnNfNfnnnnnnnnnnsnsn-3″ (SEQ ID NO: 6055), wherein “Nf” is a 2′ fluoro-modified nucleoside. “n” is a 2′ O-methyl modified nucleoside, and “s” is a phosphorothioate linkage. In some embodiments, the sense strand comprises modification pattern 42S:
5′-snnnnNfNfnnNfNfnnnnnnnnnsnsn-3″ (SEQ ID NO: 6056), wherein “Nf” is a 2′ fluoro-modified nucleoside. “n” is a 2′ O-methyl modified nucleoside, and “s” is a phosphorothioate linkage. In some embodiments, the sense strand comprises modification pattern 43S:
5-snnnnNfnNfnNfnnnnnnnnnnsnsn-3″ (SEQ ID NO: 6057), wherein “Nf” is a 2′ fluoro-modified nucleoside. “n” is a 2′ O-methyl modified nucleoside, and “s” is a phosphorothioate linkage. In some embodiments, the sense strand comprises modification pattern 44S:
5′-snnnnnnnnNfnNfnnnnnnnnsnsn-3′ (SEQ ID NO: 6058), wherein “Nf” is a 2′ fluoro-modified nucleoside. “n” is a 2′ O-methyl modified nucleoside, and “s” is a phosphorothioate linkage. In some embodiments, the sense strand comprises modification pattern 45S:
5′-snnnnnNfNfNfNfnNfnnnnnnnnsnsn-3′ (SEQ ID NO: 6059), wherein “Nf” is a 2′ fluoro-modified nucleoside. “n” is a 2′ O-methyl modified nucleoside, and “s” is a phosphorothioate linkage. In some embodiments, the sense strand comprises modification pattern 46S:
5′-snnnnnnNfnNfnNfnnnnnnnnsnsn-3′ (SEQ ID NO: 6060), wherein “Nf” is a 2′ fluoro-modified nucleoside. “n” is a 2′ O-methyl modified nucleoside, and “s” is a phosphorothioate linkage. In some embodiments, the sense strand comprises modification pattern 47S:
5′-snnnnNfnNfnNfnNfnnnnnnnnsnsn-3″ (SEQ ID NO: 6061), wherein “Nf” is a 2′ fluoro-modified nucleoside. “n” is a 2′ O-methyl modified nucleoside, and “s” is a phosphorothioate linkage. In some embodiments, the sense strand comprises modification pattern 48S:
5′-snnnnnnnnNfNfnnnnnnnnnsnsn-3′ (SEQ ID NO: 6062), wherein “Nf” is a 2′ fluoro-modified nucleoside. “n” is a 2′ O-methyl modified nucleoside, and “s” is a phosphorothioate linkage. In some embodiments, the sense strand comprises modification pattern 49S:
5″-snnnnNfnnNfNfnNfnnnnnnnnsnsn-3′ (SEQ ID NO: 6063), wherein “Nf” is a 2′ fluoro-modified nucleoside. “n” is a 2′ O-methyl modified nucleoside, and “s” is a phosphorothioate linkage. In some embodiments, the sense strand comprises modification pattern 50S:
5-snnnnnNfNfnNfnNfnnnnnnnnsnsn-3′ (SEQ ID NO: 6064), wherein “Nf is a 2” fluoro-modified nucleoside. “n” is a 2′ O-methyl modified nucleoside, and “s” is a phosphorothioate linkage.

In some embodiments, the composition comprises an oligonucleotide that inhibits the expression of PLIN1, wherein the oligonucleotide comprises an siRNA comprising a sense strand and an antisense strand, wherein the antisense strand comprises modification pattern 1AS:
5″-nsNfsnNfnNfnNfnNfnnnNfnNfnNfnsnsn-3′ (SEQ ID NO: 6065), wherein “Nf is a 2” fluoro-modified nucleoside. “n” is a 2′ O-methyl modified nucleoside, and “s” is a phosphorothioate linkage. In some embodiments, the antisense strand comprises modification pattern 2AS:
5′-nsNfsnnnNfnNfNfnnnnNfnNfinnsnsn-3″ (SEQ ID NO: 6066), wherein “Nf” is a 2′ fluoro-modified nucleoside. “n” is a 2′ O-methyl modified nucleoside, and “s” is a phosphorothioate linkage. In some embodiments, the antisense strand comprises modification pattern 3AS:
5′-nsNfsnnnNfnnnnnnnNfnNfnnnsnsn-3′ (SEQ ID NO: 6067), wherein “Nf” is a 2′ fluoro-modified nucleoside. “n” is a 2′ O-methyl modified nucleoside, and “s” is a phosphorothioate linkage. In some embodiments, the antisense strand comprises modification pattern 4AS:
5′-nsNfsnNfnNfnnnnnnnNfnNfnnnsnsn-3″ (SEQ ID NO: 6068), wherein “Nf is a 2” fluoro-modified nucleoside. “n” is a 2′ O-methyl modified nucleoside, and “s” is a phosphorothioate linkage. In some embodiments, the antisense strand comprises modification pattern 5AS:
5″-nsNfsnnnnnnnnnnnNfnNfnnnsnsn-3″ (SEQ ID NO: 6069), wherein “Nf” is a 2′ fluoro-modified nucleoside. “n” is a 2′ O-methyl modified nucleoside, and “s” is a phosphorothioate linkage. In some embodiments, the antisense strand comprises modification pattern 6AS:
5″-nsNfsnnnNfnnNfnnnnNfnNfnnnsnsn-3″ (SEQ ID NO: 6070), wherein “Nf” is a 2′ fluoro-modified nucleoside. “n” is a 2′ O-methyl modified nucleoside, and “s” is a phosphorothioate linkage. In some embodiments, the antisense strand comprises modification pattern 7AS:
5′-nsNfsnNfnNfnNfnNfnNfnNfnNfnNfnsnsn-3′ (SEQ ID NO: 6071), wherein “Nf” is a 2′ fluoro-modified nucleoside. “n” is a 2′ O-methyl modified nucleoside, and “s” is a phosphorothioate linkage. In some embodiments, the antisense strand comprises modification pattern 8AS:
5″-nsNfsnnnnnnnnnnnNfnnnnnsnsn-3′ (SEQ ID NO: 6072), wherein “Nf” is a 2′ fluoro-modified nucleoside. “n” is a 2′ O-methyl modified nucleoside, and “s” is a phosphorothioate linkage. In some embodiments, the antisense strand comprises modification pattern 9AS:
5′-5 VpnsNfsnNfnNfnNfnNfnNfnNfnNfnNfnsnsn-3″ (SEQ ID NO: 6073), wherein “Nf” is a 2′ fluoro-modified nucleoside. “n” is a 2′ O-methyl modified nucleoside. “s” is a phosphorothioate linkage, and “5Vp” is a 5′ vinylphosphonate. In some embodiments, the antisense strand comprises modification pattern 10AS: 5′-nsnsnNfnNfnNfnNfnNfnNfnNfnNfnsnsn-3′ (SEQ ID NO: 6075), wherein “Nf” is a 2′ fluoro-modified nucleoside. “n” is a 2′ O-methyl modified nucleoside, and “s” is a phosphorothioate linkage.

In some embodiments, the composition comprises an oligonucleotide that inhibits the expression of PLIN1, wherein the oligonucleotide comprises an siRNA comprising a sense strand and an antisense strand, wherein the sense strand comprises pattern 1S and the antisense strand comprises pattern 1AS, 2AS, 3AS, 4AS, 5AS, 6AS, 7AS, 8AS, 9AS or 10AS. In some embodiments, the sense strand comprises pattern 2S and the antisense strand comprises pattern 1AS, 2AS, 3AS, 4AS, 5AS. 6AS, 7AS, 8AS, 9AS or 10AS. In some embodiments, the sense strand comprises pattern 3S and the antisense strand comprises pattern 1AS, 2AS, 3AS, 4AS, 5AS, 6AS, 7AS, 8AS, 9AS or 10AS. In some embodiments, the sense strand comprises pattern 4S and the antisense strand comprises pattern 1AS, 2AS, 3AS, 4AS, 5AS, 6AS, 7AS, 8AS, 9AS or 10AS. In some embodiments, the sense strand comprises pattern 5S and the antisense strand comprises pattern 1AS, 2AS, 3AS, 4AS, 5AS, 6AS. 7AS, 8AS, 9AS or 10AS. In some embodiments, the sense strand comprises pattern 6S and the antisense strand comprises pattern 1AS, 2AS, 3AS, 4AS, 5AS, 6AS, 7AS, 8AS, 9AS or 10AS. In some embodiments, the sense strand comprises pattern 7S and the antisense strand comprises pattern 1AS, 2AS, 3AS, 4AS, 5AS, 6AS, 7AS, 8AS, 9AS or 10AS. In some embodiments, the sense strand comprises pattern 8S and the antisense strand comprises pattern 1AS, 2AS, 3AS, 4AS, 5AS, 6AS. 7AS, 8AS, 9AS or 10AS. In some embodiments, the sense strand comprises pattern 9S and the antisense strand comprises pattern 1AS, 2AS, 3AS, 4AS, 5AS, 6AS, 7AS, 8AS, 9AS or 10AS. In some embodiments, the sense strand comprises pattern 10S and the antisense strand comprises pattern 1AS, 2AS, 3AS, 4AS, 5AS, 6AS, 7AS, 8AS, 9AS or 10AS. In some embodiments, the sense strand comprises pattern 11S and the antisense strand comprises pattern 1AS, 2AS, 3AS, 4AS, 5AS, 6AS. 7AS, 8AS, 9AS or 10AS. In some embodiments, the sense strand comprises pattern 12S and the antisense strand comprises pattern 1AS, 2AS, 3AS, 4AS, 5AS, 6AS, 7AS, 8AS, 9AS or 10AS. In some embodiments, the sense strand comprises pattern 13S and the antisense strand comprises pattern 1AS, 2AS, 3AS, 4AS, 5AS, 6AS, 7AS, 8AS, 9AS or 10AS. In some embodiments, the sense strand comprises pattern 14S and the antisense strand comprises pattern 1AS, 2AS, 3AS, 4AS, 5AS, 6AS. 7AS, 8AS, 9AS or 10AS. In some embodiments, the sense strand comprises pattern 15S and the antisense strand comprises pattern 1AS, 2AS, 3AS, 4AS, 5AS, 6AS, 7AS, 8AS, 9AS or 10AS. In some embodiments, the sense strand comprises pattern 16S and the antisense strand comprises pattern 1AS, 2AS, 3AS, 4AS, 5AS, 6AS, 7AS, 8AS, 9AS or 10AS. In some embodiments, the sense strand comprises pattern 17S and the antisense strand comprises pattern 1AS, 2AS, 3AS, 4AS, 5AS, 6AS. 7AS, 8AS, 9AS or 10AS. In some embodiments, the sense strand comprises pattern 18S and the antisense strand comprises pattern 1AS, 2AS, 3AS, 4AS, 5AS, 6AS, 7AS, 8AS, 9AS or 10AS. In some embodiments, the sense strand comprises pattern 19S and the antisense strand comprises pattern 1AS, 2AS, 3AS, 4AS, 5AS, 6AS, 7AS, 8AS, 9AS or 10AS. In some embodiments, the sense strand comprises pattern 20S and the antisense strand comprises pattern 1AS, 2AS, 3AS, 4AS, 5AS, 6AS. 7AS, 8AS, 9AS or 10AS. In some embodiments, the sense strand comprises pattern 21S and the antisense strand comprises pattern 1AS, 2AS, 3AS, 4AS, 5AS, 6AS, 7AS, 8AS, 9AS or 10AS. In some embodiments, the sense strand comprises pattern 22S and the antisense strand comprises pattern 1AS, 2AS, 3AS, 4AS, 5AS, 6AS, 7AS, 8AS, 9AS or 10AS. In some embodiments, the sense strand comprises pattern 23S and the antisense strand comprises pattern 1AS, 2AS, 3AS, 4AS, 5AS, 6AS. 7AS, 8AS, 9AS or 10AS. In some embodiments, the sense strand comprises pattern 24S and the antisense strand comprises pattern 1AS, 2AS, 3AS, 4AS, 5AS, 6AS, 7AS, 8AS, 9AS or 10AS. In some embodiments, the sense strand comprises pattern 25S and the antisense strand comprises pattern 1AS, 2AS, 3AS, 4AS, 5AS, 6AS, 7AS, 8AS, 9AS or 10AS. In some embodiments, the sense strand comprises pattern 26S and the antisense strand comprises pattern 1AS, 2AS, 3AS, 4AS, 5AS, 6AS. 7AS, 8AS, 9AS or 10AS. In some embodiments, the sense strand comprises pattern 27S and the antisense strand comprises pattern 1AS, 2AS, 3AS, 4AS, 5AS, 6AS, 7AS, 8AS, 9AS or 10AS. In some embodiments, the sense strand comprises pattern 28S and the antisense strand comprises pattern 1AS, 2AS, 3AS, 4AS, 5AS, 6AS, 7AS, 8AS, 9AS or 10AS. In some embodiments, the sense strand comprises pattern 29S and the antisense strand comprises pattern 1AS, 2AS, 3AS, 4AS, 5AS, 6AS. 7AS, 8AS, 9AS or 10AS. In some embodiments, the sense strand comprises pattern 30S and the antisense strand comprises pattern 1AS, 2AS, 3AS, 4AS, 5AS, 6AS, 7AS, 8AS, 9AS or 10AS. In some embodiments, the sense strand comprises pattern 31S and the antisense strand comprises pattern 1AS, 2AS, 3AS, 4AS, 5AS, 6AS, 7AS, 8AS, 9AS or 10AS. In some embodiments, the sense strand comprises pattern 32S and the antisense strand comprises pattern 1AS, 2AS, 3AS, 4AS, 5AS, 6AS. 7AS, 8AS, 9AS or 10AS. In some embodiments, the sense strand comprises pattern 33S and the antisense strand comprises pattern 1AS, 2AS, 3AS, 4AS, 5AS, 6AS, 7AS, 8AS, 9AS or 10AS. In some embodiments, the sense strand comprises pattern 34S and the antisense strand comprises pattern 1AS, 2AS, 3AS, 4AS, 5AS, 6AS, 7AS, 8AS, 9AS or 10AS. In some embodiments, the sense strand comprises pattern 35S and the antisense strand comprises pattern 1AS, 2AS, 3AS, 4AS, 5AS, 6AS. 7AS, 8AS, 9AS or 10AS. In some embodiments, the sense strand comprises pattern 36S and the antisense strand comprises pattern 1AS, 2AS, 3AS, 4AS, 5AS, 6AS, 7AS, 8AS, 9AS or 10AS. In some embodiments, the sense strand comprises pattern 37S and the antisense strand comprises pattern 1AS, 2AS, 3AS, 4AS, 5AS, 6AS, 7AS, 8AS, 9AS or 10AS. In some embodiments, the sense strand comprises pattern 38S and the antisense strand comprises pattern 1AS, 2AS, 3AS, 4AS, 5AS, 6AS. 7AS, 8AS, 9AS or 10AS. In some embodiments, the sense strand comprises pattern 39S and the antisense strand comprises pattern 1AS, 2AS, 3AS, 4AS, 5AS, 6AS, 7AS, 8AS, 9AS or 10AS. In some embodiments, the sense strand comprises pattern 40S and the antisense strand comprises pattern 1AS, 2AS, 3AS, 4AS, 5AS, 6AS, 7AS, 8AS, 9AS or 10AS. In some embodiments, the sense strand comprises pattern 41S and the antisense strand comprises pattern 1AS, 2AS, 3AS, 4AS, 5AS, 6AS. 7AS, 8AS, 9AS or 10AS. In some embodiments, the sense strand comprises pattern 42S and the antisense strand comprises pattern 1AS, 2AS, 3AS, 4AS, 5AS, 6AS, 7AS, 8AS, 9AS or 10AS. In some embodiments, the sense strand comprises pattern 43S and the antisense strand comprises pattern 1AS, 2AS, 3AS, 4AS, 5AS, 6AS, 7AS, 8AS, 9AS or 10AS. In some embodiments, the sense strand comprises pattern 44S and the antisense strand comprises pattern 1AS, 2AS, 3AS, 4AS, 5AS, 6AS. 7AS, 8AS, 9AS or 10AS. In some embodiments, the sense strand comprises pattern 45S and the antisense strand comprises pattern 1AS, 2AS, 3AS, 4AS, 5AS, 6AS, 7AS, 8AS, 9AS or 10AS. In some embodiments, the sense strand comprises pattern 46S and the antisense strand comprises pattern 1AS, 2AS, 3AS, 4AS, 5AS, 6AS, 7AS, 8AS, 9AS or 10AS. In some embodiments, the sense strand comprises pattern 47S and the antisense strand comprises pattern 1AS, 2AS, 3AS, 4AS, 5AS, 6AS. 7AS, 8AS, 9AS or 10AS. In some embodiments, the sense strand comprises pattern 48S and the antisense strand comprises pattern 1AS, 2AS, 3AS, 4AS, 5AS, 6AS, 7AS, 8AS, 9AS or 10AS. In some embodiments, the sense strand comprises pattern 49S and the antisense strand comprises pattern 1AS, 2AS, 3AS, 4AS, 5AS, 6AS, 7AS, 8AS, 9AS or 10AS. In some embodiments, the sense strand comprises pattern 50S and the antisense strand comprises pattern 1AS, 2AS, 3AS, 4AS, 5AS, 6AS. 7AS, 8AS, 9AS or 10AS.

In some embodiments, the sense strand comprises pattern 1S, 2S, 3S, 4S, 5S, 6S, 7S, 8S, 9S. 10S, 11S, 12S, 13S, 14S, 15S, 16S, 17S, 18S, 19S, 20S, 21S, 22S, 23S, 24S, 25S, 26S, 27S, 28S. 29S, 30S, 31S, 32S, 33S, 34S, 35S, 36S, 37S, 38S, 39S, 40S, 41S, 42S, 43S, 44S, 45S, 46S, 47S, 48S. 49S, or 50S, and the antisense strand comprises pattern 1AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S. 19S, 20S, 21S, 22S, 23S, 24S, 25S, 26S, 27S, 28S, 29S, 30S, 31S, 32S, 33S, 34S, 35S, 36S, 37S, 38S. 39S, 40S, 41S, 42S, 43S, 44S, 45S, 46S, 47S, 48S, 49S, or 50S, and the antisense strand comprises pattern 2AS. In some embodiments, the sense strand comprises pattern 1S, 2S, 3S, 4S, 5S, 6S, 7S, 8S. 9S, 10S, 11S, 12S, 13S, 14S, 15S, 16S, 17S, 18S, 19S, 20S, 21S, 22S, 23S, 24S, 25S, 26S, 27S, 28S. 29S, 30S, 31S, 32S, 33S, 34S, 35S, 36S, 37S, 38S, 39S, 40S, 41S, 42S, 43S, 44S, 45S, 46S, 47S, 48S. 49S, or 50S, and the antisense strand comprises pattern 3AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S. 19S, 20S, 21S, 22S, 23S, 24S, 25S, 26S, 27S, 28S, 29S, 30S, 31S, 32S, 33S, 34S, 35S, 36S, 37S, 38S. 39S, 40S, 41S, 42S, 43S, 44S, 45S, 46S, 47S, 48S, 49S, or 50S, and the antisense strand comprises pattern 4AS. In some embodiments, the sense strand comprises pattern 1S, 2S, 3S, 4S, 5S, 6S, 7S, 8S. 9S, 10S, 11S, 12S, 13S, 14S, 15S, 16S, 17S, 18S, 19S, 20S, 21S, 22S, 23S, 24S, 25S, 26S, 27S, 28S. 29S, 30S, 31S, 32S, 33S, 34S, 35S, 36S, 37S, 38S, 39S, 40S, 41S, 42S, 43S, 44S, 45S, 46S, 47S, 48S. 49S, or 50S, and the antisense strand comprises pattern 5AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S. 19S, 20S, 21S, 22S, 23S, 24S, 25S, 26S, 27S, 28S, 29S, 30S, 31S, 32S, 33S, 34S, 35S, 36S, 37S, 38S. 39S, 40S, 41S, 42S, 43S, 44S, 45S, 46S, 47S, 48S, 49S, or 50S, and the antisense strand comprises pattern 6AS. In some embodiments, the sense strand comprises pattern 1S, 2S, 3S, 4S, 5S, 6S, 7S, 8S. 9S, 10S, 11S, 12S, 13S, 14S, 15S, 16S, 17S, 18S, 19S, 20S, 21S, 22S, 23S, 24S, 25S, 26S, 27S, 28S. 29S, 30S, 31S, 32S, 33S, 34S, 35S, 36S, 37S, 38S, 39S, 40S, 41S, 42S, 43S, 44S, 45S, 46S, 47S, 48S. 49S, or 50S, and the antisense strand comprises pattern 7AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S. 19S, 20S, 21S, 22S, 23S, 24S, 25S, 26S, 27S, 28S, 29S, 30S, 31S, 32S, 33S, 34S, 35S, 36S, 37S, 38S. 39S, 40S, 41S, 42S, 43S, 44S, 45S, 46S, 47S, 48S, 49S, or 50S, and the antisense strand comprises pattern 8AS. In some embodiments, the sense strand comprises pattern 1S, 2S, 3S, 4S, 5S, 6S, 7S, 8S. 9S, 10S, 11S, 12S, 13S, 14S, 15S, 16S, 17S, 18S, 19S, 20S, 21S, 22S, 23S, 24S, 25S, 26S, 27S, 28S. 29S, 30S, 31S, 32S, 33S, 34S, 35S, 36S, 37S, 38S, 39S, 40S, 41S, 42S, 43S, 44S, 45S, 46S, 47S, 48S. 49S, or 50S, and the antisense strand comprises pattern 9AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S. 19S, 20S, 21S, 22S, 23S, 24S, 25S, 26S, 27S, 28S, 29S, 30S, 31S, 32S, 33S, 34S, 35S, 36S, 37S, 38S. 39S, 40S, 41S, 42S, 43S, 44S, 45S, 46S, 47S, 48S, 49S, or 50S, and the antisense strand comprises pattern 10AS.

In some embodiments, the sense strand comprises modification pattern 1S, 2S, 3S, 4S, 5S, 6S. 7S, 8S, 9S, 10S, 11S, 12S, 13S, 14S, 15S, 16S, 17S, 18S, 19S, 20S, 21S, 22S, 23S, 24S, 25S, 26S. 27S, 28S, 29S, 30S, 31S, 32S, 33S, 34S, 35S, 36S, 37S, 38S, 39S, 40S, 41S, 42S, 43S, 44S, 45S, 46S. 47S, 48S, 49S, or 50S. In some embodiments, the sense strand comprises modification pattern 1S, 2S. 3S, 4S, 5S, 6S, 7S, 8S, 9S, 10S, 11S, 12S, 13S, 14S, 15S, 16S, 17S, 18S, 19S, 20S, 21S, 22S, 23S. 24S, 25S, 26S, 27S, 28S, 29S, 30S, 31S, 32S, 33S, 34S, 35S, 36S, 37S, 38S, 39S or 40S. In some embodiments, the sense strand comprises modification pattern 1AS, 2AS, 3AS, 4AS, 5AS, 6AS, 7AS. 7AS, 8AS, 9AS or 10AS. In some embodiments, the antisense strand comprises modification pattern 1AS, 2AS, 3AS, 4AS, 5AS, 6AS, 7AS, 8AS, 9AS or 10AS. In some embodiments, the antisense strand comprises modification pattern 1S, 2S, 3S, 4S, 5S, 6S, 7S, 8S, 9S, 10S, 11S, 12S, 13S, 14S. 15S, 16S, 17S, 18S, 19S, 20S, 21S, 22S, 23S, 24S, 25S, 26S, 27S, 28S, 29S, 30S, 31S, 32S, 33S, 34S. 35S, 36S, 37S, 38S, 39S, 40S, 41S, 42S, 43S, 44S, 45S, 46S, 47S, 48S, 49S, or 50S. In some embodiments, the sense strand or the antisense strand comprises modification pattern ASO1.

In some embodiments, purines of the sense strand comprise 2′ fluoro modified purines. In some embodiments, purines of the sense strand comprise 2′-O-methyl modified purines. In some embodiments, purines of the sense strand comprise a mixture of 2′ fluoro and 2′-O-methyl modified purines. In some embodiments, all purines of the sense strand comprise 2° fluoro modified purines. In some embodiments, all purines of the sense strand comprise 2″-O-methyl modified purines. In some embodiments, all purines of the sense strand comprise a mixture of 2′ fluoro and 2′-O-methyl modified purines.

In some embodiments, pyrimidines of the sense strand comprise 2′ fluoro modified pyrimidines. In some embodiments, pyrimidines of the sense strand comprise 2′-O-methyl modified pyrimidines. In some embodiments, pyrimidines of the sense strand comprise a mixture of 2′ fluoro and 2′-O-methyl modified pyrimidines. In some embodiments, all pyrimidines of the sense strand comprise 2′ fluoro modified pyrimidines. In some embodiments, all pyrimidines of the sense strand comprise 2′-O-methyl modified pyrimidines. In some embodiments, all pyrimidines of the sense strand comprise a mixture of 2′ fluoro and 2′-O-methyl modified pyrimidines.

In some embodiments, purines of the sense strand comprise 2′ fluoro modified purines, and pyrimidines of the sense strand comprise a mixture of 2′ fluoro and 2′-O-methyl modified pyrimidines. In some embodiments, purines of the sense strand comprise 2′-O-methyl modified purines, and pyrimidines of the sense strand comprise a mixture of 2′ fluoro and 2-O-methyl modified pyrimidines. In some embodiments, purines of the sense strand comprise 2′ fluoro modified purines, and pyrimidines of the sense strand comprise 2′-O-methyl modified pyrimidines. In some embodiments, purines of the sense strand comprise 2′-O-methyl modified purines, and pyrimidines of the sense strand comprise 2′ fluoro modified pyrimidines. In some embodiments, pyrimidines of the sense strand comprise 2′ fluoro modified pyrimidines, and purines of the sense strand comprise a mixture of 2′ fluoro and 2′-O-methyl modified purines. In some embodiments, pyrimidines of the sense strand comprise 2′-O-methyl modified pyrimidines, and purines of the sense strand comprise a mixture of 2′ fluoro and 2′-O-methyl modified purines. In some embodiments, pyrimidines of the sense strand comprise 2′ fluoro modified pyrimidines, and purines of the sense strand comprise 2′-O-methyl modified purines. In some embodiments, pyrimidines of the sense strand comprise 2′-O-methyl modified pyrimidines, and purines of the sense strand comprise 2′ fluoro modified purines.

In some embodiments, all purines of the sense strand comprise 2′ fluoro modified purines, and all pyrimidines of the sense strand comprise a mixture of 2′ fluoro and 2′-O-methyl modified pyrimidines. In some embodiments, all purines of the sense strand comprise 2′-O-methyl modified purines, and all pyrimidines of the sense strand comprise a mixture of 2′ fluoro and 2′-O-methyl modified pyrimidines. In some embodiments, all purines of the sense strand comprise 2′ fluoro modified purines, and all pyrimidines of the sense strand comprise 2′-O-methyl modified pyrimidines. In some embodiments, all purines of the sense strand comprise 2′-O-methyl modified purines, and all pyrimidines of the sense strand comprise 2′ fluoro modified pyrimidines. In some embodiments, all pyrimidines of the sense strand comprise 2′ fluoro modified pyrimidines, and all purines of the sense strand comprise a mixture of 2′ fluoro and 2′-O-methyl modified purines. In some embodiments, all pyrimidines of the sense strand comprise 2′-O-methyl modified pyrimidines, and all purines of the sense strand comprise a mixture of 2′ fluoro and 2′-O-methyl modified purines. In some embodiments, all pyrimidines of the sense strand comprise 2′ fluoro modified pyrimidines, and all purines of the sense strand comprise 2′-O-methyl modified purines. In some embodiments, all pyrimidines of the sense strand comprise 2′-O-methyl modified pyrimidines, and all purines of the sense strand comprise 2′ fluoro modified purines.

In some embodiments, purines of the antisense strand comprise 2′ fluoro modified purines. In some embodiments, purines of the antisense strand comprise 2′-O-methyl modified purines. In some embodiments, purines of the antisense strand comprise a mixture of 2′ fluoro and 2′-O-methyl modified purines. In some embodiments, all purines of the antisense strand comprise 2′ fluoro modified purines. In some embodiments, all purines of the antisense strand comprise 2′-O-methyl modified purines. In some embodiments, all purines of the antisense strand comprise a mixture of 2′ fluoro and 2′-O-methyl modified purines.

In some embodiments, pyrimidines of the antisense strand comprise 2′ fluoro modified pyrimidines. In some embodiments, pyrimidines of the antisense strand comprise 2″-O-methyl modified pyrimidines. In some embodiments, pyrimidines of the antisense strand comprise a mixture of 2′ fluoro and 2′-O-methyl modified pyrimidines. In some embodiments, all pyrimidines of the antisense strand comprise 2′ fluoro modified pyrimidines. In some embodiments, all pyrimidines of the antisense strand comprise 2′-O-methyl modified pyrimidines. In some embodiments, all pyrimidines of the antisense strand comprise a mixture of 2′ fluoro and 2′-O-methyl modified pyrimidines.

In some embodiments, purines of the antisense strand comprise 2′ fluoro modified purines, and pyrimidines of the antisense strand comprise a mixture of 2′ fluoro and 2′-O-methyl modified pyrimidines. In some embodiments, purines of the antisense strand comprise 2′-O-methyl modified purines, and pyrimidines of the antisense strand comprise a mixture of 2′ fluoro and 2-O-methyl modified pyrimidines. In some embodiments, purines of the antisense strand comprise 2′ fluoro modified purines, and pyrimidines of the antisense strand comprise 2′-O-methyl modified pyrimidines. In some embodiments, purines of the antisense strand comprise 2′-O-methyl modified purines, and pyrimidines of the antisense strand comprise 2′ fluoro modified pyrimidines. In some embodiments, pyrimidines of the antisense strand comprise 2′ fluoro modified pyrimidines, and purines of the antisense strand comprise a mixture of 2′ fluoro and 2′-O-methyl modified purines. In some embodiments, pyrimidines of the antisense strand comprise 2′-O-methyl modified pyrimidines, and purines of the antisense strand comprise a mixture of 2′ fluoro and 2′-O-methyl modified purines. In some embodiments, pyrimidines of the antisense strand comprise 2″ fluoro modified pyrimidines, and purines of the antisense strand comprise 2′-O-methyl modified purines. In some embodiments, pyrimidines of the antisense strand comprise 2′-O-methyl modified pyrimidines, and purines of the antisense strand comprise 2′ fluoro modified purines.

In some embodiments, all purines of the antisense strand comprise 2′ fluoro modified purines, and all pyrimidines of the antisense strand comprise a mixture of 2′ fluoro and 2′-O-methyl modified pyrimidines. In some embodiments, all purines of the antisense strand comprise 2′-O-methyl modified purines, and all pyrimidines of the antisense strand comprise a mixture of 2′ fluoro and 2′-O-methyl modified pyrimidines. In some embodiments, all purines of the antisense strand comprise 2′ fluoro modified purines, and all pyrimidines of the antisense strand comprise 2′-O-methyl modified pyrimidines. In some embodiments, all purines of the antisense strand comprise 2′-O-methyl modified purines, and all pyrimidines of the antisense strand comprise 2′ fluoro modified pyrimidines. In some embodiments, all pyrimidines of the antisense strand comprise 2° fluoro modified pyrimidines, and all purines of the antisense strand comprise a mixture of 2′ fluoro and 2′-O-methyl modified purines. In some embodiments, all pyrimidines of the antisense strand comprise 2′-O-methyl modified pyrimidines, and all purines of the antisense strand comprise a mixture of 2′ fluoro and 2-O-methyl modified purines. In some embodiments, all pyrimidines of the antisense strand comprise 2′ fluoro modified pyrimidines, and all purines of the antisense strand comprise 2′-O-methyl modified purines. In some embodiments, all pyrimidines of the antisense strand comprise 2-O-methyl modified pyrimidines, and all purines of the antisense strand comprise 2′ fluoro modified purines.

Disclosed herein, in some embodiments, are modified oligonucleotides. The modified oligonucleotide may be an siRNA that includes modifications to the ribose rings, and phosphate linkages. The modifications may be in particular patterns that maximize cell delivery, stability, and efficiency. The siRNA may also include a vinyl phosphonate and a hydrophobic group. These modifications may aid in delivery to a cell or tissue within a subject. The modified oligonucleotide may be used in a method such as a treatment method or a method of reducing gene expression.

In some embodiments, the oligonucleotide comprises a duplex consisting of 21 nucleotide single strands with base pairing between 19 of the base pairs. In some embodiments, the duplex comprises single-stranded 2 nucleotide overhangs are at the 3′ ends of each strand. One strand (antisense strand) is complementary to a PLIN1 mRNA. Each end of the antisense strand has one to two phosphorothioate bonds. The 5′ end has an optional phosphate mimic such as a vinyl phosphonate. In some embodiments, the oligonucleotide is used to knock down a PLIN1 mRNA or a target protein. In some embodiments, the sense strand has the same sequence as the PLIN1 mRNA. In some embodiments, there are 1-2 phosphorothioates at the 3′ end. In some embodiments, there are 1 or no phosphorothioates at the 5′ end. In some embodiments, there is a hydrophobic conjugate of 12 to 25 carbons attached at the 5′ end via a phosphodiester bond.

In some cases, the sense strand of any of the siRNAs comprises siRNA with a particular modification pattern. In some embodiments of the modification pattern, position 9 counting from the 5′ end of the sense strand may have a 2′F modification. In some embodiments, when position 9 of the sense strand is a pyrimidine, then all purines in the sense strand have a 2′OMe modification. In some embodiments, when position 9 is the only pyrimidine between positions 5 and 11 of the sense stand, then position 9 is the only position with a 2′F modification in the sense strand. In some embodiments, when position 9 and only one other base between positions 5 and 11 of the sense strand are pyrimidines, then both of these pyrimidines are the only two positions with a 2′F modification in the sense strand. In some embodiments, when position 9 and only two other bases between positions 5 and 11 of the sense strand are pyrimidines, and those two other pyrimidines are in adjacent positions so that there would be not three 2′F modifications in a row, then any combination of 2′F modifications can be made that give three 2′F modifications in total. In some embodiments, when there are more than 2 pyrimidines between positions 5 and 11 of the sense strand, then all combinations of pyrimidines having the 2′F modification are allowed that have three to five 2′F modifications in total, provided that the sense strand does not have three 2′F modifications in a row. In some cases, the sense strand of any of the siRNAs comprises a modification pattern which conforms to any or all of these sense strand rules.

In some embodiments, when position 9 of the sense strand is a purine, then all purines in the sense strand have a 2′OMe modification. In some embodiments, when position 9 is the only purine between positions 5 and 11 of the sense stand, then position 9 is the only position with a 2′F modification in the sense strand. In some embodiments, when position 9 and only one other base between positions 5 and 11 of the sense strand are purines, then both of these purines are the only two positions with a 2′F modification in the sense strand. In some embodiments, when position 9 and only two other bases between positions 5 and 11 of the sense strand are purines, and those two other purines are in adjacent positions so that there would be not three 2′F modifications in a row, then any combination of 2′F modifications can be made that give three 2′F modifications in total. In some embodiments, when there are more than 2 purines between positions 5 and 11 of the sense strand, then all combinations of purines having the 2′F modification are allowed that have three to five 2′F modifications in total, provided that the sense strand does not have three 2′F modifications in a row. In some cases, the sense strand of any of the siRNAs comprises a modification pattern which conforms to any or all of these sense strand rules.

In some cases, position 9 of the sense strand can be a 2′ deoxy. In these cases, 2′F and 2′OMe modifications may occur at the other positions of the sense strand. In some cases, the sense strand of any of the siRNAs comprises a modification pattern which conforms to these sense strand rules.

In some cases, the sense strand of any of the siRNAs comprises a modification pattern which conforms to these sense strand rules.

Disclosed herein, in some embodiments are compositions comprising an oligonucleotide that targets PLIN1 and when administered to a cell decreases expression of PLIN1, wherein the oligonucleotide comprises a small interfering RNA (siRNA) comprising a sense strand and an antisense strand, wherein the sense strand comprises a sense strand sequence described herein in which at least one internucleoside linkage is modified and at least one nucleoside is modified, or an sense strand sequence comprising 1 or 2 nucleoside substitutions, additions, or deletions of the oligonucleotide sequence in which at least one internucleoside linkage is modified and at least one nucleoside is modified, and wherein the antisense strand comprises an antisense strand sequence described herein in which at least one internucleoside linkage is modified and at least one nucleoside is modified, or an oligonucleotide sequence comprising 1 or 2 nucleoside substitutions, additions, or deletions of the antisense strand sequence in which at least one internucleoside linkage is modified and at least one nucleoside is modified. Some embodiments relate to methods that include administering the composition to a subject.

In some embodiments, the siRNA comprises a sense strand, an antisense strand, and a lipid moiety connected to an end of the sense or antisense strand; wherein the lipid moiety comprises a phenyl or cyclohexyl linker, wherein the linker is connected to a lipid and to the end of the sense or antisense strand. In some embodiments, any one of the following is true with regard to the sense strand; all purines comprise 2′ fluoro modified purines, and all pyrimidines comprise a mixture of 2′ fluoro and 2′-O-methyl modified pyrimidines; all purines comprise 2′-O-methyl modified purines, and all pyrimidines comprise a mixture of 2′ fluoro and 2′-O-methyl modified pyrimidines; all purines comprise 2′ fluoro modified purines, and all pyrimidines comprise 2′-O-methyl modified pyrimidines; all pyrimidines comprise 2′ fluoro modified pyrimidines, and all purines comprise a mixture of 2′ fluoro and 2′-O-methyl modified purines; all pyrimidines comprise 2′-O-methyl modified pyrimidines, and all purines comprise a mixture of 2′ fluoro and 2′-O-methyl modified purines; or all pyrimidines comprise 2′ fluoro modified pyrimidines, and all purines comprise 2′-O-methyl modified purines. In some embodiments, any one of the following is true with regard to the antisense strand; all purines comprise 2′ fluoro modified purines, and all pyrimidines comprise a mixture of 2′ fluoro and 2′-O-methyl modified pyrimidines; all purines comprise 2′-O-methyl modified purines, and all pyrimidines comprise a mixture of 2′ fluoro and 2′-O-methyl modified pyrimidines; all purines comprise 2′-O-methyl modified purines, and all pyrimidines comprise 2′ fluoro modified pyrimidines; all pyrimidines comprise 2′ fluoro modified pyrimidines, and all purines comprise a mixture of 2′ fluoro and 2′-O-methyl modified purines; all pyrimidines comprise 2′-O-methyl modified pyrimidines, and all purines comprise a mixture of 2′ fluoro and 2″-O-methyl modified purines; or all pyrimidines comprise 2′-O-methyl modified pyrimidines, and all purines comprise 2′ fluoro modified purines. In some embodiments, the siRNA comprises comprising a sense strand and an antisense strand; wherein the antisense strand comprises a 5′ end comprising a vinyl phosphonate and 2 phosphorothioate linkages, and a 3′ end comprising 2 phosphorothioate linkages; wherein the sense strand comprises a 5′ end comprising a hydrophobic moiety, and a 3′ end comprising 2 phosphorothioate linkages; wherein any one of the following is true with regard to the sense strand; all purines comprise 2′ fluoro modified purines, and all pyrimidines comprise a mixture of 2′ fluoro and 2′-O-methyl modified pyrimidines, all purines comprise 2′-O-methyl modified purines, and all pyrimidines comprise a mixture of 2′ fluoro and 2′-O-methyl modified pyrimidines, all purines comprise 2′ fluoro modified purines, and all pyrimidines comprise 2′-O-methyl modified pyrimidines, all pyrimidines comprise 2′ fluoro modified pyrimidines, and all purines comprise a mixture of 2′ fluoro and 2′-O-methyl modified purines, all pyrimidines comprise 2′-O-methyl modified pyrimidines, and all purines comprise a mixture of 2′ fluoro and 2-O-methyl modified purines, or all pyrimidines comprise 2′ fluoro modified pyrimidines, and all purines comprise 2′-O-methyl modified purines; and wherein any one of the following is true with regard to the antisense strand; all purines comprise 2′ fluoro modified purines, and all pyrimidines comprise a mixture of 2′ fluoro and 2′-O-methyl modified pyrimidines, all purines comprise 2′-O-methyl modified purines, and all pyrimidines comprise a mixture of 2′ fluoro and 2′-O-methyl modified pyrimidines, all purines comprise 2′-O-methyl modified purines, and all pyrimidines comprise 2′ fluoro modified pyrimidines, all pyrimidines comprise 2′ fluoro modified pyrimidines, and all purines comprise a mixture of 2′ fluoro and 2-O-methyl modified purines, all pyrimidines comprise 2′-O-methyl modified pyrimidines, and all purines comprise a mixture of 2′ fluoro and 2′-O-methyl modified purines, or all pyrimidines comprise 2′-O-methyl modified pyrimidines, and all purines comprise 2″ fluoro modified purines.

In some embodiments, the antisense strand comprises one or two 3′ phosphorothioate linkages. For example, there may be a phosphorothioate linkage between the first and second nucleotides from the 3′ end of the antisense strand, or there may be phosphorothioate linkages between the first, second and third nucleotides from the 3′ end of the antisense strand. In some embodiments, the sense strand comprises one or two 5′ phosphorothioate linkages. For example, there may be a phosphorothioate linkage between the first and second nucleotides from the 5′ end of the sense strand, or there may be phosphorothioate linkages between the first, second and third nucleotides from the 5′ end of the sense strand. In some embodiments, the sense strand does not comprise one or two 5′ phosphorothioate linkages. For example, in some embodiments, there are no phosphorothioate linkages between the last 3 nucleotides at the 5′ end of the sense strand. In some embodiments, the sense strand comprises 5′ phosphate linkages. In some embodiments, the sense strand comprises one or two 3′ phosphorothioate linkages. For example, there may be a phosphorothioate linkage between the first and second nucleotides from the 3′ end of the sense strand, or there may be phosphorothioate linkages between the first, second and third nucleotides from the 3″ end of the sense strand.

In some embodiments, the composition comprises an oligonucleotide that inhibits the expression of a target nucleic acid, wherein the oligonucleotide comprises an siRNA comprising a sense strand and an antisense strand, wherein the oligonucleotide comprises a hydrophobic moiety. In some embodiments, the hydrophobic moiety may be attached at the 5′ end of the sense strand. In some embodiments, the hydrophobic moiety may be attached at the 3′ end of the sense strand. In some embodiments, the hydrophobic moiety may be attached at the 5′ end of the antisense strand. In some embodiments, the hydrophobic moiety may be attached at the 3′ end of the antisense strand.

In some embodiments, the composition comprises an oligonucleotide that inhibits the expression of a target nucleic acid, wherein the oligonucleotide comprises an siRNA comprising a sense strand and an antisense strand, wherein the oligonucleotide comprises one or more vinyl phosphonate. In some embodiments, the one or more vinyl phosphonate may be attached at the 5′ end of the sense strand. In some embodiments, the one or more vinyl phosphonate may be attached at the 3′ end of the sense strand. In some embodiments, the one or more vinyl phosphonate may be attached at the 5′ end of the antisense strand. In some embodiments, the one or more vinyl phosphonate may be attached at the 3′ end of the antisense strand.

In some embodiments, the sense strand comprises or consists of RNA or modified RNA nucleotides. In some embodiments, the sense strand comprises a deoxy nucleoside. The deoxy nucleoside may include a DNA nucleoside. In some embodiments, the deoxy nucleoside comprises or consists of a 2′ deoxy nucleoside. The deoxy nucleoside may be at a position within the sense strand (5′ to 3′, where the 5′ position is 1). The position within the sense strand may be or include position 2, 4, 6, 8, 9, 10, 12, 14, 16, or 18, or a combination of said positions. The position within the sense strand may be or include position 2, 4, 6, 8, 10, 12, 14, 16, or 18, or a combination of said positions. The position within the sense strand may be or include position 2, 6, 9, 10, 14, or 18, or a combination of said positions. The position within the sense strand may be or include position 2, 6, 10, 14, or 18, or a combination of said positions. The position within the sense strand may be or include position 4, 8, 9, 12, or 16, or a combination of said positions. The position within the sense strand may be or include position 4, 8, 12, or 16, or a combination of said positions. The position within the sense strand may include position 9. The position within the sense strand may be position 9. The sense strand may include 1, 2, 3, 4, 5, 6, 7, 8, 9, 10, 11, 12, 13, 14, 15, 16, 17, 18, or 19 deoxy nucleosides. In some embodiments, the sense strand includes 1 deoxy nucleoside. The sense strand may include 1, 2, 3, 4, 5, 6, 7, 8, 9, 10, 11, 12, 13, 14, 15, 16, 17, 18, or 19 deoxy nucleosides, or a range of deoxy nucleosides defined by any two of the aforementioned numbers of deoxy nucleosides. The sense strand may include deoxy nucleosides at all even positions. The sense strand may include deoxy nucleosides at some even positions. The sense strand may include deoxy nucleosides at every other even position. The sense strand may include 1 deoxy nucleoside. The sense strand may include at least 1 deoxy nucleoside. The sense strand may include at least 2 deoxy nucleosides. The sense strand may include at least 3 deoxy nucleosides. The sense strand may include at least 4 deoxy nucleosides. The sense strand may include at least 5 deoxy nucleosides. The sense strand may include at least 6 deoxy nucleosides. The sense strand may include at least 7 deoxy nucleosides. The sense strand may include at least 8 deoxy nucleosides. The sense strand may include at least 9 deoxy nucleosides. The sense strand may include at least 10 deoxy nucleosides. The sense strand may include no greater than 2 deoxy nucleosides. The sense strand may include no greater than 3 deoxy nucleosides. The sense strand may include no greater than 4 deoxy nucleosides. The sense strand may include no greater than 5 deoxy nucleosides. The sense strand may include no greater than 6 deoxy nucleosides. The sense strand may include no greater than 7 deoxy nucleosides. The sense strand may include no greater than 8 deoxy nucleosides. The sense strand may include no greater than 9 deoxy nucleosides. The sense strand may include no greater than 10 deoxy nucleosides.

In some embodiments, the siRNA comprises the sense strand and/or the antisense strand sequence of an siRNA in Table 10, or a nucleic acid sequence thereof having 3 or 4 nucleoside substitutions, additions, or deletions. In some embodiments, the siRNA comprises the sense strand and/or the antisense strand sequence of an siRNA in Table 10, or a nucleic acid sequence thereof having 1 or 2 nucleoside substitutions, additions, or deletions. In some embodiments, the siRNA comprises the sense strand and/or the antisense strand sequence of an siRNA in Table 10. The siRNA may include the same internucleoside linkage modifications or nucleoside modifications as those in Table 10. The siRNA may include any different internucleoside linkage modifications or nucleoside modifications different from those in Table 10. The siRNA may include some unmodified internucleoside linkages or nucleosides.

In some embodiments, the siRNA comprises the sense strand and/or the antisense strand sequence of an siRNA in Table 11, or a nucleic acid sequence thereof having 3 or 4 nucleoside substitutions, additions, or deletions. In some embodiments, the siRNA comprises the sense strand and/or the antisense strand sequence of an siRNA in Table 11, or a nucleic acid sequence thereof having 1 or 2 nucleoside substitutions, additions, or deletions. In some embodiments, the siRNA comprises the sense strand and/or the antisense strand sequence of an siRNA in Table 11. The siRNA may include the same internucleoside linkage modifications or nucleoside modifications as those in Table 11. The siRNA may include any different internucleoside linkage modifications or nucleoside modifications different from those in Table 11. The siRNA may include some unmodified internucleoside linkages or nucleosides.

In some embodiments, the siRNA comprises the sense strand and/or the antisense strand sequence of an siRNA in Table 12, or a nucleic acid sequence thereof having 3 or 4 nucleoside substitutions, additions, or deletions. In some embodiments, the siRNA comprises the sense strand and/or the antisense strand sequence of an siRNA in Table 12, or a nucleic acid sequence thereof having 1 or 2 nucleoside substitutions, additions, or deletions. In some embodiments, the siRNA comprises the sense strand and/or the antisense strand sequence of an siRNA in Table 12. The siRNA may include the same internucleoside linkage modifications or nucleoside modifications as those in Table 12. The siRNA may include any different internucleoside linkage modifications or nucleoside modifications different from those in Table 12. The siRNA may include some unmodified internucleoside linkages or nucleosides.

In some embodiments, the siRNA comprises the sense strand and/or the antisense strand sequence of an siRNA in Table 14, or a nucleic acid sequence thereof having 3 or 4 nucleoside substitutions, additions, or deletions. In some embodiments, the siRNA comprises the sense strand and/or the antisense strand sequence of an siRNA in Table 14, or a nucleic acid sequence thereof having 1 or 2 nucleoside substitutions, additions, or deletions. In some embodiments, the siRNA comprises the sense strand and/or the antisense strand sequence of an siRNA in Table 14. The siRNA may include the same internucleoside linkage modifications or nucleoside modifications as those in Table 14. The siRNA may include any different internucleoside linkage modifications or nucleoside modifications different from those in Table 14. The siRNA may include some unmodified internucleoside linkages or nucleosides.

In some embodiments, the siRNA comprises the sense strand and/or the antisense strand sequence of an siRNA in Table 16, or a nucleic acid sequence thereof having 3 or 4 nucleoside substitutions, additions, or deletions. In some embodiments, the siRNA comprises the sense strand and/or the antisense strand sequence of an siRNA in Table 16, or a nucleic acid sequence thereof having 1 or 2 nucleoside substitutions, additions, or deletions. In some embodiments, the siRNA comprises the sense strand and/or the antisense strand sequence of an siRNA in Table 16. The siRNA may include the same internucleoside linkage modifications or nucleoside modifications as those in Table 16. The siRNA may include any different internucleoside linkage modifications or nucleoside modifications different from those in Table 16. The siRNA may include some unmodified internucleoside linkages or nucleosides.

In some embodiments, the siRNA comprises the sense strand and/or the antisense strand sequence of an siRNA in Table 18, or a nucleic acid sequence thereof having 3 or 4 nucleoside substitutions, additions, or deletions. In some embodiments, the siRNA comprises the sense strand and/or the antisense strand sequence of an siRNA in Table 18, or a nucleic acid sequence thereof having 1 or 2 nucleoside substitutions, additions, or deletions. In some embodiments, the siRNA comprises the sense strand and/or the antisense strand sequence of an siRNA in Table 18. The siRNA may include the same internucleoside linkage modifications or nucleoside modifications as those in Table 18. The siRNA may include any different internucleoside linkage modifications or nucleoside modifications different from those in Table 18. The siRNA may include some unmodified internucleoside linkages or nucleosides.

Some siRNAs include ETD01754 or a variant thereof. In some embodiments, the siRNA comprises a sense strand having a sequence in accordance with SEQ ID NO: 5947. In some embodiments, the sense strand sequence comprises or consists of sequence at least 75% identical to SEQ ID NO: 5947, at least 80% identical to SEQ ID NO: 5947, at least 85% identical to SEQ ID NO: 5947, at least 90% identical to SEQ ID NO: 5947, or at least 95% identical to SEQ ID NO: 5947. In some embodiments, the sense strand sequence comprises or consists of the sequence of SEQ ID NO 5947, or a sense strand sequence thereof having 1, 2, 3, or 4 nucleoside substitutions, additions, or deletions. In some embodiments, the sense strand sequence comprises or consists of the sequence of SEQ ID NO: 5947, or a sense strand sequence thereof having 1 or 2 nucleoside substitutions, additions, or deletions. In some embodiments, the sense strand sequence comprises or consists of a sequence 100% identical to SEQ ID NO: 5947. The sense strand may comprise any modifications or modification pattern described herein. The sense strand may comprise a moiety such as a GalNAc moiety or a lipid moiety. In some embodiments, the siRNA comprises an antisense strand having a sequence in accordance with SEQ ID NO: 5960. In some embodiments, the antisense strand sequence comprises or consists of sequence at least 75% identical to SEQ ID NO: 5960, at least 80% identical to SEQ ID NO: 5960, at least 85% identical to SEQ ID NO: 5960, at least 90% identical to SEQ ID NO: 5960, or at least 95% identical to SEQ ID NO: 5960. In some embodiments, the antisense strand sequence comprises or consists of the sequence of SEQ ID NO 5960, or an antisense strand sequence thereof having 1, 2, 3, or 4 nucleoside substitutions, additions, or deletions. In some embodiments, the antisense strand sequence comprises or consists of the sequence of SEQ ID NO: 5960, or an antisense strand sequence thereof having 1 or 2 nucleoside substitutions, additions, or deletions. In some embodiments, the antisense strand sequence comprises or consists of a sequence 100% identical to SEQ ID NO: 5960. The antisense strand may comprise any modifications or modification pattern described herein. The antisense strand may comprise a moiety such as a GalNAc moiety or a lipid moiety.

Some siRNAs include ETD01900 or a variant thereof. In some embodiments, the siRNA comprises a sense strand having a sequence in accordance with SEQ ID NO: 5951. In some embodiments, the sense strand sequence comprises or consists of sequence at least 75% identical to SEQ ID NO: 5951, at least 80% identical to SEQ ID NO: 5951, at least 85% identical to SEQ ID NO: 5951, at least 90% identical to SEQ ID NO: 5951, or at least 95% identical to SEQ ID NO: 5951. In some embodiments, the sense strand sequence comprises or consists of the sequence of SEQ ID NO 5951, or a sense strand sequence thereof having 1, 2, 3, or 4 nucleoside substitutions, additions, or deletions. In some embodiments, the sense strand sequence comprises or consists of the sequence of SEQ ID NO: 5951, or a sense strand sequence thereof having 1 or 2 nucleoside substitutions, additions, or deletions. In some embodiments, the sense strand sequence comprises or consists of a sequence 100% identical to SEQ ID NO: 5951. The sense strand may comprise any modifications or modification pattern described herein. The sense strand may comprise a moiety such as a GalNAc moiety or a lipid moiety. In some embodiments, the siRNA comprises an antisense strand having a sequence in accordance with SEQ ID NO: 5964. In some embodiments, the antisense strand sequence comprises or consists of sequence at least 75% identical to SEQ ID NO: 5964, at least 80% identical to SEQ ID NO: 5964, at least 85% identical to SEQ ID NO: 5964, at least 90% identical to SEQ ID NO: 5964, or at least 95% identical to SEQ ID NO: 5964. In some embodiments, the antisense strand sequence comprises or consists of the sequence of SEQ ID NO 5964, or an antisense strand sequence thereof having 1, 2, 3, or 4 nucleoside substitutions, additions, or deletions. In some embodiments, the antisense strand sequence comprises or consists of the sequence of SEQ ID NO: 5964, or an antisense strand sequence thereof having 1 or 2 nucleoside substitutions, additions, or deletions. In some embodiments, the antisense strand sequence comprises or consists of a sequence 100% identical to SEQ ID NO: 5964. The antisense strand may comprise any modifications or modification pattern described herein. The antisense strand may comprise a moiety such as a GalNAc moiety or a lipid moiety.

Some siRNAs include ETD01901 or a variant thereof. In some embodiments, the siRNA comprises a sense strand having a sequence in accordance with SEQ ID NO: 5952. In some embodiments, the sense strand sequence comprises or consists of sequence at least 75% identical to SEQ ID NO: 5952, at least 80% identical to SEQ ID NO: 5952, at least 85% identical to SEQ ID NO: 5952, at least 90% identical to SEQ ID NO: 5952, or at least 95% identical to SEQ ID NO: 5952. In some embodiments, the sense strand sequence comprises or consists of the sequence of SEQ ID NO 5952, or a sense strand sequence thereof having 1, 2, 3, or 4 nucleoside substitutions, additions, or deletions. In some embodiments, the sense strand sequence comprises or consists of the sequence of SEQ ID NO: 5952, or a sense strand sequence thereof having 1 or 2 nucleoside substitutions, additions, or deletions. In some embodiments, the sense strand sequence comprises or consists of a sequence 100% identical to SEQ ID NO: 5952. The sense strand may comprise any modifications or modification pattern described herein. The sense strand may comprise a moiety such as a GalNAc moiety or a lipid moiety. In some embodiments, the siRNA comprises an antisense strand having a sequence in accordance with SEQ ID NO: 5965. In some embodiments, the antisense strand sequence comprises or consists of sequence at least 75% identical to SEQ ID NO: 5965, at least 80% identical to SEQ ID NO: 5965, at least 85% identical to SEQ ID NO: 5965, at least 90% identical to SEQ ID NO: 5965, or at least 95% identical to SEQ ID NO: 5965. In some embodiments, the antisense strand sequence comprises or consists of the sequence of SEQ ID NO 5965, or an antisense strand sequence thereof having 1, 2, 3, or 4 nucleoside substitutions, additions, or deletions. In some embodiments, the antisense strand sequence comprises or consists of the sequence of SEQ ID NO: 5965, or an antisense strand sequence thereof having 1 or 2 nucleoside substitutions, additions, or deletions. In some embodiments, the antisense strand sequence comprises or consists of a sequence 100% identical to SEQ ID NO: 5965. The antisense strand may comprise any modifications or modification pattern described herein. The antisense strand may comprise a moiety such as a GalNAc moiety or a lipid moiety.

Some siRNAs include ETD01902 or a variant thereof. In some embodiments, the siRNA comprises a sense strand having a sequence in accordance with SEQ ID NO: 5953. In some embodiments, the sense strand sequence comprises or consists of sequence at least 75% identical to SEQ ID NO: 5953, at least 80% identical to SEQ ID NO: 5953, at least 85% identical to SEQ ID NO: 5953, at least 90% identical to SEQ ID NO: 5953, or at least 95% identical to SEQ ID NO: 5953. In some embodiments, the sense strand sequence comprises or consists of the sequence of SEQ ID NO 5953, or a sense strand sequence thereof having 1, 2, 3, or 4 nucleoside substitutions, additions, or deletions. In some embodiments, the sense strand sequence comprises or consists of the sequence of SEQ ID NO: 5953, or a sense strand sequence thereof having 1 or 2 nucleoside substitutions, additions, or deletions. In some embodiments, the sense strand sequence comprises or consists of a sequence 100% identical to SEQ ID NO: 5953. The sense strand may comprise any modifications or modification pattern described herein. The sense strand may comprise a moiety such as a GalNAc moiety or a lipid moiety. In some embodiments, the siRNA comprises an antisense strand having a sequence in accordance with SEQ ID NO: 5966. In some embodiments, the antisense strand sequence comprises or consists of sequence at least 75% identical to SEQ ID NO: 5966, at least 80% identical to SEQ ID NO: 5966, at least 85% identical to SEQ ID NO: 5966, at least 90% identical to SEQ ID NO: 5966, or at least 95% identical to SEQ ID NO: 5966. In some embodiments, the antisense strand sequence comprises or consists of the sequence of SEQ ID NO 5966, or an antisense strand sequence thereof having 1, 2, 3, or 4 nucleoside substitutions, additions, or deletions. In some embodiments, the antisense strand sequence comprises or consists of the sequence of SEQ ID NO: 5966, or an antisense strand sequence thereof having 1 or 2 nucleoside substitutions, additions, or deletions. In some embodiments, the antisense strand sequence comprises or consists of a sequence 100% identical to SEQ ID NO: 5966. The antisense strand may comprise any modifications or modification pattern described herein. The antisense strand may comprise a moiety such as a GalNAc moiety or a lipid moiety.

### 4. ASO Modification Patterns

In some embodiments, the composition comprises an oligonucleotide that inhibits the expression of PLIN1, wherein the oligonucleotide comprises an antisense oligonucleotide (ASO). In some embodiments, the ASO comprises modification pattern ASO1:

5″-nsnsnsnsnsdNsdNsdNsdNsdNsdNsdNsdNsdNsdNsnsnsnsnsn-3′ (SEQ ID NO: 6075), wherein “dN” is any deoxynucleotide, “n” is a 2′O-methyl or 2′O-methoxyethyl-modified nucleoside, and “'s” is a phosphorothioate linkage. In some embodiments, the ASO comprises modification 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 1AS, 2AS, 3AS, 4AS, 5AS, 6AS, 7AS, 8AS, 9AS or 10AS.

### D. Formulations

In some embodiments, the composition is a pharmaceutical composition. In some embodiments, the composition is sterile. In some embodiments, the composition further comprises a pharmaceutically acceptable carrier.

In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

### II. Methods and Uses

Disclosed herein, in some embodiments, are methods of administering a composition described herein to a subject. Some embodiments relate to use a composition described herein, such as administering the composition to a subject.

Some embodiments relate to a method of treating a disorder in a subject in need thereof. Some embodiments relate to use of a composition described herein in the method of treatment. Some embodiments include administering a composition described herein to a subject with the disorder. In some embodiments, the administration treats the disorder in the subject. In some embodiments, the composition treats the disorder in the subject.

In some embodiments, the treatment comprises prevention, inhibition, or reversion of the disorder in the subject. Some embodiments relate to use of a composition described herein in the method of preventing, inhibiting, or reversing the disorder. Some embodiments relate to a method of preventing, inhibiting, or reversing a disorder a disorder in a subject in need thereof. Some embodiments include administering a composition described herein to a subject with the disorder. In some embodiments, the administration prevents, inhibits, or reverses the disorder in the subject. In some embodiments, the composition prevents, inhibits, or reverses the disorder in the subject.

Some embodiments relate to a method of preventing a disorder a disorder in a subject in need thereof. Some embodiments relate to use of a composition described herein in the method of preventing the disorder. Some embodiments include administering a composition described herein to a subject with the disorder. In some embodiments, the administration prevents the disorder in the subject. In some embodiments, the composition prevents the disorder in the subject.

Some embodiments relate to a method of inhibiting a disorder a disorder in a subject in need thereof. Some embodiments relate to use of a composition described herein in the method of inhibiting the disorder. Some embodiments include administering a composition described herein to a subject with the disorder. In some embodiments, the administration inhibits the disorder in the subject. In some embodiments, the composition inhibits the disorder in the subject.

Some embodiments relate to a method of reversing a disorder a disorder in a subject in need thereof. Some embodiments relate to use of a composition described herein in the method of reversing the disorder. Some embodiments include administering a composition described herein to a subject with the disorder. In some embodiments, the administration reverses the disorder in the subject. In some embodiments, the composition reverses the disorder in the subject.

In some embodiments, the administration is systemic. In some embodiments, the administration is intravenous. In some embodiments, the administration is by injection.

### A. Disorders

Some embodiments of the methods described herein include treating a disorder in a subject in need thereof. In some embodiments, the disorder is a cardiometabolic disorder. The cardiometabolic disorder may comprise a cardiovascular disorder, a cerebrovascular disorder, a hypertensive disorder, or a metabolic disorder, or a combination thereof.

In some embodiments, the disorder comprises a cardiovascular disorder. Non-limiting examples of cardiovascular disorders include coronary artery disease, peripheral vascular disease, peripheral arterial disease, myocardial infarction, heart failure, or hypertension. In some embodiments, the cardiovascular disorder includes coronary artery disease. In some embodiments, the cardiovascular disorder includes peripheral vascular disease. In some embodiments, the cardiovascular disorder includes peripheral arterial disease. In some embodiments, the cardiovascular disorder includes myocardial infarction. In some embodiments, the cardiovascular disorder includes heart failure. In some embodiments, the cardiovascular disorder includes hypertension.

In some embodiments, the disorder comprises a cerebrovascular disorder. A non-limiting example of a cerebrovascular disorder may include a stroke.

In some embodiments, the disorder comprises a metabolic disorder. Non-limiting examples of metabolic disorders include hyperlipidemia, hypertriglyceridemia, diabetes, or a liver disease such as non-alcoholic fatty liver disease (NAFLD) or non-alcoholic steatohepatitis (NASH). In some embodiments, the metabolic disorder includes hyperlipidemia. In some embodiments, the metabolic disorder includes hypertriglyceridemia. In some embodiments, the metabolic disorder includes diabetes. In some embodiments, the metabolic disorder includes a liver disease. In some embodiments, the liver disease includes NAFLD. In some embodiments, the liver disease includes NASH.

In some embodiments, the disorder comprises a hypertensive disorder. The hypertensive disorder may include hypertension.

The disorder may include hyperlipidemia, hypertriglyceridemia, coronary artery disease, myocardial infarction, heart failure, stroke, hypertension, diabetes, non-alcoholic fatty liver disease (NAFLD), or non-alcoholic steatohepatitis (NASH), or a combination thereof. In some embodiments, the disorder comprises hyperlipidemia. In some embodiments, the disorder comprises hypertriglyceridemia. In some embodiments, the disorder comprises coronary artery disease. In some embodiments, the disorder comprises peripheral vascular disease. In some embodiments, the disorder comprises peripheral arterial disease. In some embodiments, the disorder comprises myocardial infarction. In some embodiments, the disorder comprises heart failure. In some embodiments, the disorder comprises stroke. In some embodiments, the disorder comprises hypertension. In some embodiments, the disorder comprises diabetes. In some embodiments, the disorder comprises a liver disease. In some embodiments, the disorder comprises NAFLD. In some embodiments, the disorder comprises NASH.

### B. Subjects

Some embodiments of the methods described herein include treatment of a subject. Non-limiting examples of subjects include vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is a dog. In some embodiments, the subject is a cat. In some embodiments, the subject is a cattle. In some embodiments, the subject is a mouse. In some embodiments, the subject is a rat. In some embodiments, the subject is a primate. In some embodiments, the subject is a monkey. In some embodiments, the subject is an animal, a mammal, a dog, a cat, cattle, a rodent, a mouse, a rat, a primate, or a monkey. In some embodiments, the subject is a human.

In some embodiments, the subject is male. In some embodiments, the subject is female. In some embodiments, the subject is an adult (e.g. at least 18 years old).

In some embodiments, the subject has a body mass index (BMI) of 25, 26, 27, 28, 29, 30, 31, 32, 33, 34, 35, 36, 37, 38, 39, 40, 41, 42, 43, 44, 45, 46, 47, 48, 49, 50, or more, or a range defined by any two of the aforementioned integers. In some embodiments, the subject is overweight. In some embodiments, the subject has a BMI of 25 or more. In some embodiments, the subject has a BMI of 25-29. In some embodiments, the subject is obese. In some embodiments, the subject has a BMI of 30 or more. In some embodiments, the subject has a BMI of 30-39. In some embodiments, the subject has a BMI of 40-50. In some embodiments, the subject has a BMI of 25-50.

### C. Baseline Measurements

Some embodiments of the methods described herein include obtaining a baseline measurement from a subject. For example, in some embodiments, a baseline measurement is obtained from the subject prior to treating the subject. Non-limiting examples of baseline measurements include a baseline total cholesterol measurement, a baseline non-high density lipoprotein (HDL) cholesterol measurement, a baseline low density lipoprotein (LDL) measurement, a baseline triglyceride measurement, a baseline hemoglobin A1c measurement, a baseline Apolipoprotein B (APOB) measurement, a baseline glucose measurement, a baseline systolic blood pressure measurement, a baseline diastolic blood pressure measurement, a baseline alanine aminotransferase (ALT) measurement, a baseline aspartate aminotransferase (AST) measurement, a baseline blood alkaline phosphatase (ALP) measurement, a baseline gamma-glutamyl transferase (GGT) measurement, a baseline liver fibrosis score, a baseline nonalcoholic fatty liver disease (NAFLD) fibrosis score, a baseline NAFLD activity score, a baseline liver fat percentage measurement, a baseline HDL measurement, a baseline apolipoprotein A1 (ApoA1) measurement, a baseline insulin sensitivity measurement, a baseline left ventricular ejection fraction measurement, a baseline PLIN1 protein measurement, or a baseline PLIN1 mRNA measurement.

In some embodiments, the baseline measurement is obtained directly from the subject. In some embodiments, the baseline measurement is obtained by observation, for example by observation of the subject or of the subject's tissue. In some embodiments, the baseline measurement is obtained noninvasively using an imaging device.

In some embodiments, the baseline measurement is obtained in a sample from the subject. In some embodiments, the baseline measurement is obtained in one or more histological tissue sections. In some embodiments, the baseline measurement is obtained by performing an assay such as an immunoassay, a colorimetric assay, or a fluorescence assay, on the sample obtained from the subject. In some embodiments, the baseline measurement is obtained by an immunoassay, a colorimetric assay, a fluorescence assay, or a chromatography (e.g. HPLC) assay. In some embodiments, the baseline measurement is obtained by PCR.

In some embodiments, the baseline measurement is a baseline cholesterol measurement. In some embodiments, the baseline cholesterol concentration is a baseline total cholesterol measurement. In some embodiments, the baseline cholesterol concentration is a baseline non-high density lipoprotein (HDL) cholesterol measurement. In some embodiments, the baseline cholesterol concentration is a baseline low density lipoprotein (LDL) cholesterol measurement. In some embodiments, the baseline cholesterol measurement is a baseline cholesterol concentration. In some embodiments, the baseline cholesterol measurement is a baseline circulating cholesterol measurement. In some embodiments, the baseline cholesterol measurement is a baseline blood cholesterol measurement. In some embodiments, the baseline cholesterol measurement is obtained by an assay such as an immunoassay, a colorimetric assay, or a fluorescence assay.

In some embodiments, the baseline measurement is a baseline low density lipoprotein (LDL) measurement. In some embodiments, the baseline LDL measurement comprises a baseline very low density lipoprotein (VLDL) measurement. In some embodiments, the baseline LDL measurement is a baseline LDL concentration. In some embodiments, the baseline LDL measurement is a baseline circulating LDL measurement. In some embodiments, the baseline LDL measurement is a baseline blood LDL measurement. In some embodiments, the baseline LDL measurement is obtained by an assay such as an immunoassay, a colorimetric assay, or a fluorescence assay.

In some embodiments, the baseline measurement is a baseline high density lipoprotein (HDL) measurement. In some embodiments, the baseline HDL measurement is a baseline HDL concentration. In some embodiments, the baseline HDL measurement is a baseline circulating HDL measurement. In some embodiments, the baseline HDL measurement is a baseline blood HDL measurement. In some embodiments, the baseline HDL measurement is obtained by an assay such as an immunoassay, a colorimetric assay, or a fluorescence assay.

In some embodiments, the baseline measurement is a baseline apolipoprotein A1 (ApoA1) measurement. In some embodiments, the baseline ApoA1 measurement is a baseline ApoA1 concentration. In some embodiments, the baseline ApoA1 measurement is a baseline circulating ApoA1 measurement. In some embodiments, the baseline ApoA1 measurement is a baseline blood ApoA1 measurement. In some embodiments, the baseline ApoA1 measurement is obtained by an assay such as an immunoassay, a colorimetric assay, or a fluorescence assay.

In some embodiments, the baseline measurement is a baseline triglyceride measurement. In some embodiments, the baseline triglyceride measurement is a baseline triglyceride concentration (for example, mg/dL). In some embodiments, the baseline triglyceride measurement is a baseline circulating triglyceride measurement. In some embodiments, the baseline triglyceride measurement a baseline circulating triglyceride measurement above 150 mg/dL. In some embodiments, the baseline triglyceride measurement is obtained by an assay such as an immunoassay, a colorimetric assay, or a fluorescence assay.

In some embodiments, the baseline measurement is a baseline hemoglobin A1C measurement. In some embodiments, the baseline hemoglobin A1C measurement is a baseline hemoglobin A1C concentration. In some embodiments, the baseline hemoglobin A1C measurement is a baseline circulating hemoglobin A1C measurement. In some embodiments, the baseline hemoglobin A1C measurement is obtained by an assay such as an immunoassay, a colorimetric assay, a fluorescence assay, or HPLC. The baseline hemoglobin A1C measurement may be indicative of a healthy normal A1C measurement. The healthy normal hemoglobin A1C measurement may be below 48 mmol/mol (6.5 DCCT %). The healthy normal hemoglobin A1C measurement may be below 53 mmol/mol (7.0) DCCT %). The baseline hemoglobin A1C measurement may be indicative of diabetes of pre-diabetes. A baseline hemoglobin A1C measurement above 48 mmol/mol, or above 53 mmol/mol may indicate diabetes of pre-diabetes. The baseline hemoglobin A1C measurement may be indicative of diabetes. The baseline hemoglobin A1C measurement may be indicative of pre-diabetes. In some cases, the baseline hemoglobin A1C measurement is below 5.7 DCCT % (e.g. indicative of a normal healthy diagnosis). In some cases, the baseline hemoglobin A1C measurement is between 5.7 and 6.4 DCCT % (e.g. indicative of prediabetes). In some cases, the baseline hemoglobin A1C measurement is above 6.4 DCCT % (e.g. indicative of diabetes).

In some embodiments, the baseline measurement is a baseline apolipoprotein B (APOB) measurement. In some embodiments, the baseline APOB measurement is a baseline APOB concentration. In some embodiments, the baseline APOB measurement comprises a baseline APOB concentration. In some embodiments, the baseline APOB measurement is a baseline circulating APOB measurement. In some embodiments, the baseline APOB measurement is obtained by an assay such as an immunoassay, a colorimetric assay, or a fluorescence assay.

In some embodiments, the baseline measurement is a baseline glucose measurement. In some embodiments, the baseline glucose measurement is a baseline glucose concentration (for example, mg/dL). In some embodiments, the baseline glucose measurement comprises a baseline glucose concentration. In some embodiments, the baseline glucose measurement is a baseline circulating glucose measurement. In some embodiments, the baseline glucose measurement is obtained by an assay such as an immunoassay, a colorimetric assay, or a fluorescence assay.

In some embodiments, the baseline glucose measurement comprises a baseline glucose tolerance test. In some embodiments, the baseline glucose tolerance test comprises administering glucose to the subject, and then obtaining multiple baseline glucose measurements over time after administering the glucose to the subject. In some embodiments, the glucose is administered orally. In some embodiments, the glucose is administered by injection. In some embodiments, the multiple baseline glucose measurements are integrated into a baseline glucose area under the curve (AUC) measurement. In some embodiments, the baseline glucose tolerance test is performed on the subject in a fasted state such as after an overnight fast. In some embodiments, the baseline glucose measurement comprises a baseline glucose measurement other than a baseline glucose tolerance test.

In some embodiments, the baseline measurement is a baseline insulin measurement. In some embodiments, the baseline insulin measurement is a baseline insulin sensitivity measurement. In some embodiments, the baseline insulin sensitivity measurement is obtained using a glucose clamp technique such as a hyperinsulinemic euglycemic clamp. In some embodiments, the baseline insulin measurement is a baseline insulin concentration. In some embodiments, the baseline insulin measurement comprises a baseline insulin concentration. In some embodiments, the baseline insulin measurement is a baseline circulating insulin measurement. In some embodiments, the baseline insulin measurement is obtained by an assay such as an immunoassay (for example, an ELISA or an immunoblot), a colorimetric assay, or a fluorescence assay. In some embodiments, the baseline insulin sensitivity measurement comprises a baseline glucose tolerance test. In some embodiments, the baseline insulin sensitivity measurement comprises a baseline insulin sensitivity measurement other than a baseline glucose tolerance test.

In some embodiments, the baseline insulin measurement comprises a baseline insulin response test. In some embodiments, the baseline insulin response test comprises administering glucose to the subject and then obtaining multiple baseline insulin measurements over time after administering the glucose to the subject. In some embodiments, the glucose is administered orally. In some embodiments, the glucose is administered by injection. In some embodiments, the multiple baseline insulin measurements are integrated into a baseline insulin AUC measurement. In some embodiments, the baseline insulin response test is performed on the subject in a fasted state such as after an overnight fast.

In some embodiments, the baseline insulin measurement comprises a baseline glucose response test. In some embodiments, the baseline glucose response test comprises administering insulin to the subject, and then obtaining multiple baseline glucose measurements over time after administering the insulin to the subject. In some embodiments, the insulin is administered by injection. In some embodiments, the multiple baseline glucose measurements are integrated into a baseline glucose AUC measurement. In some embodiments, the multiple baseline glucose measurements are obtained with a glucometer. In some embodiments, the glucose response test is performed on the subject in a fasted state such as after an overnight fast. In some embodiments, the glucose response test is performed on the subject after administering food, drink or glucose to the subject.

Some embodiments of the methods described herein include obtaining the baseline measurement of the subject by measuring blood pressure (e.g. systolic or diastolic) with a sphygmomanometer in which a healthcare professional places a cuff around an arm of the subject and inflates the cuff with a pump until the circulation is cut off. A small valve slowly deflates the cuff, and the healthcare professional measures the pressure with the aid of a stethoscope that is placed over the arm of the subject in order to listen for the sound of the blood pulsing through the arteries. The first measurement in which blood rushes is the systolic blood pressure (SBP), and after the sound fades, the second number indicates the diastolic blood pressure (DBP), which is a measure the blood pressure of the heart at rest. The mean arterial pressure (MAP) is an average blood pressure of the subject during a single cardiac cycle. The MAP can be measured directly using methods such as applanation tonometry or it can be approximated by using a formula in which the diastolic blood pressure is doubled and added to the systolic blood pressure and that composite sum is then divided by 3 to estimate MAP.

In some embodiments, the baseline measurement is a baseline systolic blood (SBP) pressure measurement. In some embodiments, the baseline SBP measurement is measured in mm of mercury (mm Hg). In some embodiments, the SBP measurement is obtained with a sphygmomanometer. The baseline SBP measurement may be indicative of normal blood pressure. For most adults, normal SBP at rest is within the range of 100-130 mmHg. For most adults, hypertension is present if the resting blood pressure is persistently at or above 130/80 or 140/90 mmHg. The baseline SBP measurement may be indicative of hypertension (e.g. at least 130 mmHg, or at least 140 mmHg). The baseline SBP measurement may include a baseline cerebral SBP measurement.

In some embodiments, the baseline measurement is a baseline diastolic blood (DBP) pressure measurement. In some embodiments, the baseline DBP measurement is measured in mm Hg. In some embodiments, the DBP measurement is obtained with a sphygmomanometer. The baseline DBP measurement may be indicative of normal blood pressure. For most adults, normal DBP at rest is within the range of 60-80 mmHg. The baseline DBP measurement may be indicative of hypertension (e.g. at least 80 mmHg, or at least 90 mmHg). The baseline DBP measurement may include a baseline cerebral DBP measurement.

In some embodiments, the baseline measurement is a baseline systolic heart function measurement. A baseline heart systolic function measurement may include a measure of heart pumping capacity. An example of a baseline systolic function measurement includes a baseline ejection fraction measurement. A baseline ejection fraction measurement may include a baseline left ventricular ejection fraction measurement, a baseline right ventricular ejection fraction measurement, a baseline left atrial ejection fraction measurement, or a baseline right atrial ejection fraction measurement. In some embodiments, the baseline ejection fraction measurement includes a baseline left ventricular ejection fraction measurement. A subject with heart failure, for example, may have a left ventricular ejection fraction below 60%, below 50%, below 40%, below 30%, below 20%, or below: 10%. In some embodiments, a baseline left ventricular ejection fraction below 35% is indicative of systolic dysfunction. Another example of a baseline systolic heart function measurement is a baseline cardiac output measurement. The baseline systolic heart function measurement may be measured using a medical imaging device such as an ultrasound (e.g., an echocardiography device) or magnetic resonance imaging device.

In some embodiments, the baseline measurement is a baseline liver enzyme measurement. In some embodiments, the baseline liver enzyme measurement is a baseline alanine aminotransferase (ALT) measurement. In some embodiments, the baseline liver enzyme measurement is a baseline aspartate aminotransferase (AST) measurement. In some embodiments, the baseline liver enzyme measurement comprises an ALT/AST ratio, or comprises an AST/ALT ratio.

In some embodiments, the baseline measurement is a baseline alanine aminotransferase (ALT) measurement. In some embodiments, the baseline ALT measurement is a baseline ALT concentration (for example. Units/dL). In some embodiments, the baseline ALT measurement is a baseline blood ALT measurement, for example, a baseline blood, serum, or plasma ALT level. In some embodiments, the baseline ALT measurement is obtained by an assay such as an immunoassay, a colorimetric assay, or a fluorescence assay.

In some embodiments, the baseline measurement is a baseline aspartate aminotransferase (AST) measurement. In some embodiments, the baseline AST measurement is a baseline AST concentration (for example. Units/L). In some embodiments, the baseline AST measurement is a baseline blood AST measurement, for example, a baseline blood, serum, or plasma AST level. In some embodiments, the baseline AST measurement is obtained by an assay such as an immunoassay, a colorimetric assay, or a fluorescence assay.

In some embodiments, the baseline measurement is a baseline alkaline phosphatase (ALP) measurement. In some embodiments, the baseline ALP measurement is a baseline ALP concentration. In some embodiments, the baseline ALP measurement is a baseline blood ALP measurement. In some embodiments, the baseline ALP measurement is obtained by an assay such as an immunoassay, a colorimetric assay, a chromatography assay, or a fluorescence assay.

In some embodiments, the baseline measurement is a baseline gamma-glutamyl transferase (GGT) measurement. In some embodiments, the baseline GGT measurement is a baseline GGT concentration. In some embodiments, the baseline GGT measurement is a baseline blood GGT measurement. In some embodiments, the baseline GGT measurement is obtained by an assay such as an immunoassay, a colorimetric assay, a chromatography assay, or a fluorescence assay.

In some embodiments, the baseline measurement is a baseline liver fibrosis measurement. In some embodiments, the baseline liver fibrosis measurement is a baseline liver fibrosis score (LFS). In some embodiments, the baseline LFS comprises a score of 0, 1, 2, 3, or 4, or a range of scores defined by any two of the aforementioned numbers. In some embodiments, the baseline LFS comprises a score of 0-4. In some embodiments, the baseline LFS is obtained using a scoring system exemplified in Table 2. In some embodiments, the baseline LFS measurement is obtained noninvasively. In some embodiments, the baseline LFS measurement is obtained by a medical imaging device such as a vibration-controlled transient elastography (VCTE) device, a shear wave elastography device, a medical resonance imaging (MRI) device, a magnetic resonance spectroscopy device, a computed tomography device, or an ultrasound device. In some embodiments, the baseline LFS measurement is obtained in a liver sample. In some embodiments, the baseline LFS is obtained by an assay such as an immunoassay, a colorimetric assay, or a fluorescence assay. In some embodiments, the baseline LFS is obtained using one or more indirect markers or measures of liver fibrosis such as an aspartate aminotransferase-to-platelet ratio index (APRI), a Fibrosis-4 (FIB-4) index, a FibroIndex, a Forns Index, a Hepascore, or a FibroTest. In some embodiments, the baseline LFS is obtained using one or more indirect markers or measures of liver fibrosis such as a FIBROSpect test or a FIBROSpect II test. In some embodiments, the baseline LFS is obtained by RT-qPCR or RNA sequencing of one or more fibrosis-related genes such as a collagen gene. In some embodiments, the baseline LFS or the baseline LFS is obtained using a scoring system upon a visual inspection of a sample such as a histological sample. In some embodiments, the baseline LFS or the baseline LFS is obtained using a stain with an affinity to collagen.

TABLE 2
Non-Limiting Examples of Liver Fibrosis Scoring Systems

| Score | IASL | Batts-Ludwig | Metavir |
|---|---|---|---|
| 0 | No fibrosis | No fibrosis | No fibrosis |
| 1 | Mild fibrosis | Fibrous portal | Periportal fibrotic |
| | | expansion | expansion |
| 2 | Moderate fibrosis | Rare bridges or | Periportal septae |
| | | septae | (>1 septum) |
| 3 | Severe fibrosis | Numerous bridges | Portal-central septae |
| | | or septae | |
| 4 | Cirrhosis | Cirrhosis | Cirrhosis |

In some embodiments, the baseline liver fibrosis measurement is a baseline nonalcoholic fatty liver disease (NAFLD) fibrosis score. A baseline NAFLD fibrosis score may take into account laboratory test values such as platelet count, albumin, and AST/ALT ratio, and patient characteristics such as BMI, and diabetes status. A baseline NAFLD fibrosis score below −1.455 may be indicative of no fibrosis, mild fibrosis, or moderate fibrosis. A baseline NAFLD fibrosis score between-1.455 and 0.675 may be indicative of severe fibrosis. A baseline NAFLD fibrosis score above 0.675 may be indicative of cirrhosis.

In some embodiments, the baseline measurement is a baseline non-alcoholic fatty liver disease (NAFLD) activity score. In some embodiments, the baseline NAFLD activity score comprises a numerical value such as a number of points. In some embodiments, the numerical value is 0, 1, 2, 3, 4, 5, 6, 7, or 8, or a range defined by any two of the aforementioned numerical values. In some embodiments, the numerical value is 0-8. In some embodiments, the baseline NAFLD activity score comprises a steatosis grade such as a baseline liver fat percentage. In some embodiments, a steatosis grade <5% comprises 0 points in the baseline NAFLD activity score. In some embodiments, a steatosis grade of 5-33% comprises 1 point in the baseline NAFLD activity score. In some embodiments, a steatosis grade of 34-66% comprises 2 points in the baseline NAFLD activity score. In some embodiments, a steatosis grade of >66% comprises 3 points in the baseline NAFLD activity score. In some embodiments, the baseline NAFLD activity score comprises a lobular inflammation grade. In some embodiments, the lobular inflammation grade comprises an assessment of inflammatory foci. In some embodiments, a lobular inflammation grade comprising 0 foci comprises 0 points in the baseline NAFLD activity score. In some embodiments, a lobular inflammation grade comprising 1 focus per a field (such as a 20× field or a 200× field) comprises 1 point in the baseline NAFLD activity score. In some embodiments, a lobular inflammation grade comprising 2-4 foci per field comprises 2 points in the baseline NAFLD activity score. In some embodiments, a lobular inflammation grade comprising >4 foci per field comprises 3 points in the baseline NAFLD activity score. In some embodiments, the baseline NAFLD activity score comprises a liver cell injury grade such as an amount of ballooning cells. In some embodiments, a liver cell injury comprising no ballooning cells comprises 0 points in the baseline NAFLD activity score. In some embodiments, a liver cell injury comprising some new balloon cells comprises 1 points in the baseline NAFLD activity score. In some embodiments, a liver cell injury comprising many ballooning cells or prominent ballooning comprises 2 points in the baseline NAFLD activity score. In some embodiments, the baseline NAFLD activity score is obtained invasively, based on histology, and/or in a liver biopsy.

In some embodiments, the baseline measurement is a baseline liver steatosis measurement. In some embodiments, the baseline liver steatosis measurement is a baseline liver fat percentage (LFP) measurement. In some embodiments, the baseline measurement is a baseline LFP measurement. In some embodiments, the baseline LFP measurement is indicated as a mass/mass percentage of fat/total tissue. In some embodiments, the baseline LFP measurement is indicated as a mass/volume percentage of fat/total tissue. In some embodiments, the baseline LFP measurement is indicated as a volume/mass percentage of fat/total tissue. In some embodiments, the baseline LFP measurement is indicated as a volume/volume percentage of fat/total tissue. In some embodiments, the baseline LFP measurement is indicated as a score. In some embodiments, the baseline LFP measurement is obtained noninvasively. In some embodiments, the baseline LFP measurement is obtained by a medical imaging device. In some embodiments, the baseline LFP measurement is obtained by a device such as a medical resonance imaging (MRI) device, a magnetic resonance spectroscopy device a computed tomography device, a controlled attenuation parameter (CAP), a transient elastography device, or an ultrasound device. In some embodiments, the baseline LFP measurement is obtained in a liver sample. In some embodiments, the baseline LFP measurement comprises a baseline liver triglyceride measurement. In some embodiments, the baseline LFP measurement is obtained by an assay such as an immunoassay, a colorimetric assay, or a fluorescence assay. In some embodiments, the baseline LFP measurement or the baseline LFP measurement is obtained using a scoring system upon a visual inspection of a sample such as a histological sample. In some embodiments, the baseline LFP measurement or the baseline LFP measurement is obtained using a stain with an affinity to fats, such as a lysochrome diazo dye.

In some embodiments, the baseline measurement is a baseline PLIN1 protein measurement. In some embodiments, the baseline PLIN1 protein measurement comprises a baseline PLIN1 protein level. In some embodiments, the baseline PLIN1 protein level is indicated as a mass or percentage of PLIN1 protein per sample weight. In some embodiments, the baseline PLIN1 protein level is indicated as a mass or percentage of PLIN1 protein per sample volume. In some embodiments, the baseline PLIN1 protein level is indicated as a mass or percentage of PLIN1 protein per total protein within the sample. In some embodiments, the baseline PLIN1 protein measurement is a baseline tissue PLIN1 protein measurement. In some embodiments, the baseline PLIN1 protein measurement is obtained by an assay such as an immunoassay, a colorimetric assay, or a fluorescence assay.

In some embodiments, the baseline measurement is a baseline PLIN1 mRNA measurement. In some embodiments, the baseline PLIN1 mRNA measurement comprises a baseline PLIN1 mRNA level. In some embodiments, the baseline PLIN1 mRNA level is indicated as an amount or percentage of PLIN1 mRNA per sample weight. In some embodiments, the baseline PLIN1 mRNA level is indicated as an amount or percentage of PLIN1 mRNA per sample volume. In some embodiments, the baseline PLIN1 mRNA level is indicated as an amount or percentage of PLIN1 mRNA per total mRNA within the sample. In some embodiments, the baseline PLIN1 mRNA level is indicated as an amount or percentage of PLIN1 mRNA per total nucleic acids within the sample. In some embodiments, the baseline PLIN1 mRNA level is indicated relative to another mRNA level, such as an mRNA level of a housekeeping gene, within the sample. In some embodiments, the baseline PLIN1 mRNA measurement is a baseline tissue PLIN1 mRNA measurement. In some embodiments, the baseline PLIN1 mRNA measurement is obtained by an assay such as a polymerase chain reaction (PCR) assay. In some embodiments, the PCR comprises quantitative PCR (qPCR). In some embodiments, the PCR comprises reverse transcription of the PLIN1 mRNA.

Some embodiments of the methods described herein include obtaining a sample from a subject. In some embodiments, the baseline measurement is obtained in a sample obtained from the subject. In some embodiments, the sample is obtained from the subject prior to administration or treatment of the subject with a composition described herein. In some embodiments, a baseline measurement is obtained in a sample obtained from the subject prior to administering the composition to the subject. In some embodiments, the sample is obtained from the subject in a fasted state. In some embodiments, the sample is obtained from the subject after an overnight fasting period. In some embodiments, the sample is obtained from the subject in a fed state.

In some embodiments, the sample comprises a fluid. In some embodiments, the sample is a fluid sample. In some embodiments, the sample is a blood, plasma, or serum sample. In some embodiments, the sample comprises blood. In some embodiments, the sample is a blood sample. In some embodiments, the sample is a whole-blood sample. In some embodiments, the blood is fractionated or centrifuged. In some embodiments, the sample comprises plasma. In some embodiments, the sample is a plasma sample. A blood sample may be a plasma sample. In some embodiments, the sample comprises serum. In some embodiments, the sample is a serum sample. A blood sample may be a serum sample.

In some embodiments, the sample comprises a tissue. In some embodiments, the sample is a tissue sample. In some embodiments, the tissue comprises adipose, liver, brain, vascular, or heart tissue. For example, the baseline PLIN1 mRNA measurement, or the baseline PLIN1 protein measurement, may be obtained in a adipose or liver sample obtained from the patient. In some embodiments, the tissue comprises adipose tissue. In some embodiments, the adipose tissue comprises white adipose tissue. The adipose tissue may include adipocytes. In some embodiments, the tissue comprises liver tissue. The liver may include hepatocytes. In some embodiments, the tissue comprises brain tissue. In some embodiments, the tissue comprises vascular tissue. In some embodiments, the tissue comprises heart tissue. The heart tissue may include cardiomyocytes.

In some embodiments, the sample includes cells. In some embodiments, the sample comprises a cell. In some embodiments, the cell comprises an adipose cell, a liver cell, a brain cell, a vasculature cell, or a heart cell. In some embodiments, the cell is an adipose cell. In some embodiments, the adipose cell is an adipocyte. In some embodiments, the cell is a liver cell. In some embodiments, the liver cell is a hepatocyte. In some embodiments, the cell is a brain cell. In some embodiments, the cell is a vasculature cell. In some embodiments, the cell is a heart cell. In some embodiments, the heart cell is a cardiomyocyte.

### D. Effects

In some embodiments, the composition or administration of the composition affects a measurement such as a total cholesterol measurement, a non-high density lipoprotein (HDL) cholesterol measurement, a low density lipoprotein (LDL) measurement, a triglyceride measurement, a hemoglobin A1c measurement, a glucose measurement, an APOB measurement, a systolic blood pressure measurement, a diastolic blood pressure measurement, an alanine aminotransferase (ALT) measurement, an aspartate aminotransferase (AST) measurement, a blood alkaline phosphatase (ALP) measurement, a gamma-glutamyl transferase (GGT) measurement, a liver fibrosis score, a nonalcoholic fatty liver disease (NAFLD) fibrosis score, an NAFLD activity score, a liver fat percentage measurement, an HDL measurement, an apolipoprotein A1 (ApoA1) measurement, an insulin sensitivity measurement, a left ventricular ejection fraction measurement, an PLIN1 protein measurement, or an PLIN1 mRNA measurement, relative to the baseline measurement.

Some embodiments of the methods described herein include obtaining the measurement from a subject. For example, the measurement may be obtained from the subject after treating the subject. In some embodiments, the measurement is obtained in a second sample (such as a fluid or tissue sample described herein) obtained from the subject after the composition is administered to the subject. In some embodiments, the measurement is an indication that the disorder has been treated.

In some embodiments, the measurement is obtained directly from the subject. In some embodiments, the measurement is obtained noninvasively using an imaging device. In some embodiments, the measurement is obtained in a second sample from the subject. In some embodiments, the measurement is obtained in one or more histological tissue sections. In some embodiments, the measurement is obtained by performing an assay on the second sample obtained from the subject. In some embodiments, the measurement is obtained by an assay, such as an assay described herein. In some embodiments, the assay is an immunoassay, a colorimetric assay, a fluorescence assay, a chromatography (e.g. HPLC) assay, or a PCR assay. In some embodiments, the measurement is obtained by an assay such as an immunoassay, a colorimetric assay, a fluorescence assay, or a chromatography (e.g. HPLC) assay. In some embodiments, the measurement is obtained by PCR. In some embodiments, the measurement is obtained by histology. In some embodiments, the measurement is obtained by observation. In some embodiments, additional measurements are made, such as in a 3rd sample, a 4th sample, or a fifth sample.

In some embodiments, the measurement is obtained within 1 hour, within 2 hours, within 3 hours, within 4 hours, within 5 hours, within 6 hours, within 12 hours, within 18 hours, or within 24 hours after the administration of the composition. In some embodiments, the measurement is obtained within 1 day, within 2 days, within 3 days, within 4 days, within 5 days, within 6 days, or within 7 days after the administration of the composition. In some embodiments, the measurement is obtained within 1 week, within 2 weeks, within 3 weeks, within 1 month, within 2 months, within 3 months, within 6 months, within 1 year, within 2 years, within 3 years, within 4 years, or within 5 years after the administration of the composition. In some embodiments, the measurement is obtained after 1 hour, after 2 hours, after 3 hours, after 4 hours, after 5 hours, after 6 hours, after 12 hours, after 18 hours, or after 24 hours after the administration of the composition. In some embodiments, the measurement is obtained after 1 day, after 2 days, after 3 days, after 4 days, after 5 days, after 6 days, or after 7 days after the administration of the composition. In some embodiments, the measurement is obtained after 1 week, after 2 weeks, after 3 weeks, after 1 month, after 2 months, after 3 months, after 6 months, after 1 year, after 2 years, after 3 years, after 4 years, or after 5 years, following the administration of the composition.

In some embodiments, the composition reduces the measurement relative to the baseline measurement. For example, an adverse phenotype of a cardiometabolic disorder may be reduced upon administration of the composition. In some embodiments, the reduction is measured in a second sample obtained from the subject after administering the composition to the subject. In some embodiments, the reduction is measured directly in the subject after administering the composition to the subject. In some embodiments, the measurement is decreased by about 2.5% or more, about 5% or more, or about 7.5% or more, relative to the baseline measurement. In some embodiments, the measurement is decreased by about 10% or more, relative to the baseline measurement. In some embodiments, the measurement is decreased by about 20% or more, about 30% or more, about 40% or more, about 50% or more, about 60% or more, about 70% or more, about 80% or more, about 90% or more, relative to the baseline measurement. In some embodiments, the measurement is decreased by no more than about 2.5%, no more than about 5%, or no more than about 7.5%, relative to the baseline measurement. In some embodiments, the measurement is decreased by no more than about 10%, relative to the baseline measurement. In some embodiments, the measurement is decreased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the composition increases the measurement relative to the baseline measurement. For example, a protective cardiometabolic phenotype may be increased upon administration of the composition. In some embodiments, the increase is measured in a second sample obtained from the subject after administering the composition to the subject. In some embodiments, the increase is measured directly in the subject after administering the composition to the subject. In some embodiments, the measurement is increased by about 2.5% or more, about 5% or more, or about 7.5% or more, relative to the baseline measurement. In some embodiments, the measurement is increased by about 10% or more, relative to the baseline measurement. In some embodiments, the measurement is increased by about 20% or more, about 30% or more, about 40% or more, about 50% or more, about 60% or more, about 70% or more, about 80% or more, about 90% or more, relative to the baseline measurement. In some embodiments, the measurement is increased by about 100% or more, increased by about 250% or more, increased by about 500% or more, increased by about 750% or more, or increased by about 1000% or more, relative to the baseline measurement. In some embodiments, the measurement is increased by no more than about 2.5%, no more than about 5%, or no more than about 7.5%, relative to the baseline measurement. In some embodiments, the measurement is increased by no more than about 10%, relative to the baseline measurement. In some embodiments, the measurement is increased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is increased by no more than about 100%, increased by no more than about 250%, increased by no more than about 500%, increased by no more than about 750%, or increased by no more than about 1000%, relative to the baseline measurement. In some embodiments, the measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a cholesterol measurement. In some embodiments, the cholesterol concentration is a total cholesterol measurement. In some embodiments, the cholesterol concentration is a non-high density lipoprotein (HDL) cholesterol measurement. In some embodiments, the cholesterol concentration is a low density lipoprotein (LDL) cholesterol measurement. In some embodiments, the cholesterol measurement is a cholesterol concentration. In some embodiments, the cholesterol measurement is a circulating cholesterol measurement. In some embodiments, the cholesterol measurement is a blood cholesterol measurement. In some embodiments, the cholesterol measurement is obtained by an assay such as an immunoassay, a colorimetric assay, or a fluorescence assay.

In some embodiments, the composition reduces the cholesterol measurement relative to the baseline cholesterol measurement. In some embodiments, the reduction is measured in a second sample obtained from the subject after administering the composition to the subject. In some embodiments, the cholesterol measurement is decreased by about 2.5% or more, about 5% or more, or about 7.5% or more, relative to the baseline cholesterol measurement. In some embodiments, the cholesterol measurement is decreased by about 10% or more, relative to the baseline cholesterol measurement. In some embodiments, the cholesterol measurement is decreased by about 20% or more, about 30)% or more, about 40% or more, about 50% or more, about 60% or more, about 70% or more, about 80% or more, about 90% or more, relative to the baseline cholesterol measurement. In some embodiments, the cholesterol measurement is decreased by no more than about 2.5%, no more than about 5%, or no more than about 7.5%, relative to the baseline cholesterol measurement. In some embodiments, the cholesterol measurement is decreased by no more than about 10%, relative to the baseline cholesterol measurement. In some embodiments, the cholesterol measurement is decreased by no more than about 20%, no more than about 30%, no more than about 40%, no more than about 50%, no more than about 60%, no more than about 70%, no more than about 80%, or no more than about 90%, relative to the baseline cholesterol measurement. In some embodiments, the cholesterol measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a low density lipoprotein (LDL) measurement. In some embodiments, the LDL measurement comprises a very low density lipoprotein (VLDL) measurement. In some embodiments, the LDL measurement is a LDL concentration. In some embodiments, the LDL measurement is a circulating LDL measurement. In some embodiments, the LDL measurement is a blood LDL measurement. In some embodiments, the LDL measurement is obtained by an assay such as an immunoassay, a colorimetric assay, or a fluorescence assay.

In some embodiments, the composition reduces the LDL measurement relative to the baseline LDL measurement. In some embodiments, the reduction is measured in a second sample obtained from the subject after administering the composition to the subject. In some embodiments, the LDL measurement is decreased by about 2.5% or more, about 5% or more, or about 7.5% or more, relative to the baseline LDL measurement. In some embodiments, the LDL measurement is decreased by about 10% or more, relative to the baseline LDL measurement. In some embodiments, the LDL measurement is decreased by about 20% or more, about 30% or more, about 40% or more, about 50% or more, about 60% or more, about 70% or more, about 80% or more, about 90% or more, relative to the baseline LDL measurement. In some embodiments, the LDL measurement is decreased by no more than about 2.5%, no more than about 5%, or no more than about 7.5%, relative to the baseline LDL measurement. In some embodiments, the LDL measurement is decreased by no more than about 10%, relative to the baseline LDL measurement. In some embodiments, the LDL measurement is decreased by no more than about 20%, no more than about 30%, no more than about 40%, no more than about 50%, no more than about 60%, no more than about 70%, no more than about 80%, or no more than about 90%, relative to the baseline LDL measurement. In some embodiments, the LDL measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a high density lipoprotein (HDL) measurement. In some embodiments, the HDL measurement is an HDL concentration. In some embodiments, the HDL measurement is a circulating HDL measurement. In some embodiments, the HDL measurement is a blood HDL measurement. In some embodiments, the HDL measurement is obtained by an assay such as an immunoassay, a colorimetric assay, or a fluorescence assay.

In some embodiments, the composition increases the HDL measurement relative to the baseline HDL measurement. In some embodiments, the increase is measured in a second sample obtained from the subject after administering the composition to the subject. In some embodiments, the HDL measurement is increased by about 2.5% or more, about 5% or more, or about 7.5% or more relative to the baseline HDL measurement. In some embodiments, the HDL measurement is increased by about 10% or more, relative to the baseline HDL measurement. In some embodiments, the HDL measurement is increased by about 20% or more, about 30% or more, about 40% or more, about 50% or more, about 60% or more, about 70% or more, about 80% or more, about 90% or more, relative to the baseline HDL measurement. In some embodiments, the HDL measurement is increased by about 100% or more, increased by about 250% or more, increased by about 500% or more, increased by about 750% or more, or increased by about 1000% or more, relative to the baseline HDL measurement. In some embodiments, the HDL measurement is increased by no more than about 2.5%, no more than about 5%, or no more than about 7.5%, relative to the baseline HDL measurement. In some embodiments, the HDL measurement is increased by no more than about 10%, relative to the baseline HDL measurement. In some embodiments, the HDL measurement is increased by no more than about 20%, no more than about 30%, no more than about 40%, no more than about 50%, no more than about 60%, no more than about 70%, no more than about 80%, no more than about 90%, or no more than about 100% relative to the baseline HDL measurement. In some embodiments, the HDL measurement is increased by no more than about 100%, increased by no more than about 250%, increased by no more than about 500%, increased by no more than about 750%, or increased by no more than about 1000%, relative to the baseline HDL measurement. In some embodiments, the HDL measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a apolipoprotein A1 (ApoA1) measurement. In some embodiments, the ApoA1 measurement is an ApoA1 concentration. In some embodiments, the ApoA1 measurement is a circulating ApoA1 measurement. In some embodiments, the ApoA1 measurement is a blood ApoA1 measurement. In some embodiments, the ApoA1 measurement is obtained by an assay such as an immunoassay, a colorimetric assay, or a fluorescence assay.

In some embodiments, the composition increases the ApoA1 measurement relative to the baseline ApoA1 measurement. In some embodiments, the increase is measured in a second sample obtained from the subject after administering the composition to the subject. In some embodiments, the ApoA1 measurement is increased by about 2.5% or more, about 5% or more, or about 7.5% or more, relative to the baseline ApoA1 measurement. In some embodiments, the ApoA1 measurement is increased by about 10% or more, relative to the baseline ApoA1 measurement. In some embodiments, the ApoA1 measurement is increased by about 20% or more, about 30% or more, about 40% or more, about 50% or more, about 60% or more, about 70% or more, about 80% or more, about 90% or more, relative to the baseline ApoA1 measurement. In some embodiments, the ApoA1 measurement is increased by about 100% or more, increased by about 250% or more, increased by about 500% or more, increased by about 750% or more, or increased by about 1000% or more, relative to the baseline ApoA1 measurement. In some embodiments, the ApoA1 measurement is increased by no more than about 2.5%, no more than about 5%, or no more than about 7.5%, relative to the baseline ApoA1 measurement. In some embodiments, the ApoA1 measurement is increased by no more than about 10%, relative to the baseline ApoA1 measurement. In some embodiments, the ApoA1 measurement is increased by no more than about 20%, no more than about 30%, no more than about 40%, no more than about 50%, no more than about 60%, no more than about 70%, no more than about 80%, no more than about 90%, or no more than about 100% relative to the baseline ApoA1 measurement. In some embodiments, the ApoA1 measurement is increased by no more than about 100%, increased by no more than about 250%, increased by no more than about 500%, increased by no more than about 750%, or increased by no more than about 1000%, relative to the baseline ApoA1 measurement. In some embodiments, the ApoA1 measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a triglyceride measurement. In some embodiments, the triglyceride measurement is a triglyceride concentration (for example, mg/dL). In some embodiments, the triglyceride measurement is a circulating triglyceride measurement. In some embodiments, the triglyceride measurement is obtained by an assay such as an immunoassay, a colorimetric assay, or a fluorescence assay.

In some embodiments, the composition reduces the triglyceride measurement relative to the baseline triglyceride measurement. In some embodiments, the composition reduces circulating triglycerides relative to the baseline triglyceride measurement. In some embodiments, the reduced triglycerides are measured in a second sample obtained from the subject after administering the composition to the subject. In some embodiments, the triglyceride measurement is decreased by about 2.5% or more, about 5% or more, or about 7.5% or more, relative to the baseline triglyceride measurement. In some embodiments, the triglyceride measurement is decreased by about 10% or more, relative to the baseline triglyceride measurement. In some embodiments, the triglyceride measurement is decreased by about 20% or more, about 30% or more, about 40% or more, about 50% or more, about 60% or more, about 70% or more, about 80% or more, about 90% or more, or about 100%, relative to the baseline triglyceride measurement. In some embodiments, the triglyceride measurement is decreased by no more than about 2.5%, no more than about 5%, or no more than about 7.5%, relative to the baseline triglyceride measurement. In some embodiments, the triglyceride measurement is decreased by no more than about 10%, relative to the baseline triglyceride measurement. In some embodiments, the triglyceride measurement is decreased by no more than about 20%, no more than about 30%, no more than about 40%, no more than about 50%, no more than about 60%, no more than about 70%, no more than about 80%, no more than about 90%, or no more than about 100% relative to the baseline triglyceride measurement. In some embodiments, the triglyceride measurement is decreased by 2.5%, 5%, 7.5%, 10%, 20%, 30%, 40%, 50%, 60%, 70%, 80%, 90%, 100%, or by a range defined by any of the two aforementioned percentages.

In some embodiments, the measurement is a hemoglobin A1C measurement. In some embodiments, the hemoglobin A1C measurement is a hemoglobin A1C concentration. In some embodiments, the hemoglobin A1C measurement is a circulating hemoglobin A1C measurement. In some embodiments, the hemoglobin A1C measurement is obtained by an assay such as an immunoassay, a colorimetric assay, a fluorescence assay, or HPLC. The hemoglobin A1C measurement may be indicative of a healthy normal A1C measurement. The hemoglobin A1C measurement may be indicative of diabetes. The hemoglobin A1C measurement may be indicative of pre-diabetes.

In some embodiments, the composition reduces the hemoglobin A1C measurement relative to the baseline hemoglobin A1C measurement. In some embodiments, the reduction is measured in a second fluid sample obtained from the subject after administering the composition to the subject. In some embodiments, the reduction is measured directly in the subject after administering the composition to the subject. In some embodiments, the hemoglobin A1C measurement is decreased by about 2.5% or more, about 5% or more, or about 7.5% or more, relative to the baseline hemoglobin A1C measurement. In some embodiments, the hemoglobin A1C measurement is decreased by about 10% or more, relative to the baseline hemoglobin A1C measurement. In some embodiments, the hemoglobin A1C measurement is decreased by about 20% or more, about 30% or more, about 40% or more, about 50% or more, about 60% or more, about 70% or more, about 80% or more, about 90% or more, relative to the baseline hemoglobin A1C measurement. In some embodiments, the hemoglobin A1C measurement is decreased by no more than about 2.5%, no more than about 5%, or no more than about 7.5%, relative to the baseline hemoglobin A1C measurement. In some embodiments, the hemoglobin A1C measurement is decreased by no more than about 10%, relative to the baseline hemoglobin A1C measurement. In some embodiments, the hemoglobin A1C measurement is decreased by no more than about 20%, no more than about 30%, no more than about 40%, no more than about 50%, no more than about 60%, no more than about 70%, no more than about 80%, or no more than about 90% relative to the baseline hemoglobin A1C measurement. In some embodiments, the hemoglobin A1C measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a apolipoprotein B (APOB) measurement. In some embodiments, the APOB measurement is a APOB concentration. In some embodiments, the APOB measurement comprises a APOB concentration. In some embodiments, the APOB measurement is a circulating APOB measurement. In some embodiments, the APOB measurement is obtained by an assay such as an immunoassay, a colorimetric assay, or a fluorescence assay.

In some embodiments, the composition reduces the APOB measurement relative to the baseline APOB measurement. In some embodiments, the reduction is measured in a second fluid sample obtained from the subject after administering the composition to the subject. In some embodiments, the reduction is measured directly in the subject after administering the composition to the subject. In some embodiments, the APOB measurement is decreased by about 2.5% or more, about 5% or more, or about 7.5% or more, relative to the baseline APOB measurement. In some embodiments, the APOB measurement is decreased by about 10% or more, relative to the baseline APOB measurement. In some embodiments, the APOB measurement is decreased by about 20% or more, about 30% or more, about 40% or more, about 50% or more, about 60% or more, about 70% or more, about 80% or more, about 90% or more, relative to the baseline APOB measurement. In some embodiments, the APOB measurement is decreased by no more than about 2.5%, no more than about 5%, or no more than about 7.5%, relative to the baseline APOB measurement. In some embodiments, the APOB measurement is decreased by no more than about 10%, relative to the baseline APOB measurement. In some embodiments, the APOB measurement is decreased by no more than about 20%, no more than about 30%, no more than about 40%, no more than about 50%, no more than about 60%, no more than about 70%, no more than about 80%, or no more than about 90% relative to the baseline APOB measurement. In some embodiments, the APOB measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a glucose measurement. In some embodiments, the glucose measurement comprises a glucose concentration (for example, mg/dL). In some embodiments, the glucose measurement is a glucose concentration. In some embodiments, the glucose measurement is a circulating glucose measurement. In some embodiments, the glucose measurement is obtained by an assay such as an immunoassay, a colorimetric assay, or a fluorescence assay. In some embodiments, the glucose measurement is obtained using a glucometer.

In some embodiments, the glucose measurement comprises a glucose tolerance test. In some embodiments, the glucose tolerance test comprises administering glucose to the subject, and then obtaining multiple glucose measurements over time after administering the glucose to the subject. In some embodiments, the glucose is administered orally. In some embodiments, the glucose is administered by injection. In some embodiments, the multiple glucose measurements are integrated into a glucose area under the curve (AUC) measurement. In some embodiments, the glucose tolerance test is performed on the subject in a fasted state such as after an overnight fast. In some embodiments, the glucose measurement comprises a glucose measurement other than a glucose tolerance test.

In some embodiments, the composition reduces the glucose measurement relative to the baseline glucose measurement. In some embodiments, the composition reduces circulating glucose relative to the baseline glucose measurement. In some embodiments, the reduced glucose is measured in a second sample obtained from the subject after administering the composition to the subject. In some embodiments, the composition reduces one or more of the multiple glucose measurements of the glucose tolerance test relative to one or more of the multiple glucose measurements of the baseline glucose tolerance test. In some embodiments, the composition reduces the glucose AUC measurement relative to the baseline glucose AUC measurement. In some embodiments, the glucose measurement is decreased by about 2.5% or more, about 5% or more, or about 7.5% or more, relative to the baseline glucose measurement. In some embodiments, the glucose measurement is decreased by about 10% or more, relative to the baseline glucose measurement. In some embodiments, the glucose measurement is decreased by about 20% or more, about 30% or more, about 40% or more, about 50% or more, about 60% or more, about 70% or more, about 80% or more, about 90% or more, or about 100%, relative to the baseline glucose measurement. In some embodiments, the glucose is decreased by no more than about 2.5%, no more than about 5%, or no more than about 7.5%, relative to the baseline glucose measurement. In some embodiments, the glucose is decreased by no more than about 10%, relative to the baseline glucose measurement. In some embodiments, the glucose is decreased by no more than about 20%, no more than about 30%, no more than about 40%, no more than about 50%, no more than about 60%, no more than about 70%, no more than about 80%, no more than about 90%, or no more than about 100% relative to the baseline glucose measurement. In some embodiments, the glucose measurement is decreased by 2.5%, 5%, 7.5%, 10%, 20%, 30%, 40%, 50%, 60%, 70%, 80%, 90%, 100%, or by a range defined by any of the two aforementioned percentages.

In some embodiments, the measurement is an insulin measurement. In some embodiments, the insulin measurement is an insulin sensitivity measurement. In some embodiments, the insulin sensitivity measurement is obtained using a glucose clamp technique such as a hyperinsulinemic euglycemic clamp. In some embodiments, the insulin measurement comprises an insulin concentration. In some embodiments, the insulin measurement is an insulin concentration. In some embodiments, the insulin measurement is a circulating insulin measurement. In some embodiments, the insulin measurement is obtained by an assay such as an immunoassay, a colorimetric assay, or a fluorescence assay. In some embodiments, the insulin sensitivity measurement comprises a glucose tolerance test. In some embodiments, the insulin sensitivity measurement comprises an insulin sensitivity measurement other than a glucose tolerance test.

In some embodiments, the insulin measurement comprises an insulin response test. In some embodiments, the insulin response test comprises administering glucose to the subject, and then obtaining multiple insulin measurements over time after administering the glucose to the subject. In some embodiments, the glucose is administered orally. In some embodiments, the glucose is administered by injection. In some embodiments, the multiple insulin measurements are integrated into an insulin AUC measurement. In some embodiments, the insulin response test is performed on the subject in a fasted state such as after an overnight fast.

In some embodiments, the insulin measurement comprises a glucose response test. In some embodiments, the glucose response test comprises administering insulin to the subject, and then obtaining multiple glucose measurements over time after administering the insulin to the subject. In some embodiments, the insulin is administered by injection. In some embodiments, the multiple glucose measurements are integrated into a glucose AUC measurement. In some embodiments, the multiple glucose measurements are measured with a glucometer. In some embodiments, the glucose response test is performed on the subject in a fasted state such as after an overnight fast. In some embodiments, the glucose response test is performed on the subject after administering food, drink, or glucose to the subject.

In some embodiments, the composition increases the insulin sensitivity relative to the baseline insulin sensitivity measurement. In some embodiments, the insulin sensitivity is increased by about 2.5% or more, about 5% or more, or about 7.5% or more, relative to the baseline insulin sensitivity measurement. In some embodiments, the insulin sensitivity is increased by about 10% or more, relative to the baseline insulin sensitivity measurement. In some embodiments, the insulin sensitivity is increased by about 20% or more, about 30% or more, about 40% or more, about 50% or more, about 60% or more, about 70% or more, about 80% or more, about 90% or more, or about 100% or more relative to the baseline insulin sensitivity measurement. In some embodiments, the insulin sensitivity is increased by about 200% or more, about 300% or more, about 400% or more, about 500% or more, about 600% or more, about 700% or more, about 800% or more, about 900% or more, or about 1000% or more, relative to the baseline insulin sensitivity measurement. In some embodiments, the insulin sensitivity is increased by no more than about 2.5%, no more than about 5%, or no more than about 7.5%, relative to the baseline insulin sensitivity measurement. In some embodiments, the insulin sensitivity is increased by no more than about 10%, relative to the baseline insulin sensitivity measurement. In some embodiments, the insulin sensitivity is increased by no more than about 20%, no more than about 30%, no more than about 40%, no more than about 50%, no more than about 60%, no more than about 70%, no more than about 80%, no more than about 90%, or no more than about 100% relative to the baseline insulin sensitivity measurement. In some embodiments, the insulin sensitivity is increased by no more than about 200%, no more than about 300%, no more than about 400%, no more than about 500%, no more than about 600%, no more than about 700%, no more than about 800%, no more than about 900%, or no more than about 1000%, relative to the baseline insulin sensitivity measurement. In some embodiments, the insulin sensitivity is increased by 2.5%, 5%, 7.5%, 10%, 20%, 30%, 40%, 50%, 60%, 70%, 80%, 90%, 100%, 200% 300%, 400%, 500%, 600%, 700%, 800%, 900%, 1000%, or by a range defined by any of the two aforementioned percentages.

In some embodiments, the composition reduces the insulin measurement relative to the baseline insulin measurement. In some embodiments, the composition reduces circulating insulin relative to the baseline insulin measurement. In some embodiments, the reduced insulin is measured in a second sample obtained from the subject after administering the composition to the subject. In some embodiments, the composition reduces the insulin AUC measurement relative to the baseline insulin AUC measurement.

In some embodiments, the insulin measurement is decreased by about 2.5% or more, about 5% or more, or about 7.5% or more, relative to the baseline insulin measurement. In some embodiments, the insulin measurement is decreased by about 10% or more, relative to the baseline insulin measurement. In some embodiments, the insulin measurement is decreased by about 20% or more, about 30% or more, about 40% or more, about 50% or more, about 60% or more, about 70% or more, about 80% or more, about 90% or more, or about 100%, relative to the baseline insulin measurement. In some embodiments, the insulin is decreased by no more than about 2.5%, no more than about 5%, or no more than about 7.5%, relative to the baseline insulin measurement. In some embodiments, the insulin is decreased by no more than about 10%, relative to the baseline insulin measurement. In some embodiments, the insulin is decreased by no more than about 20%, no more than about 30%, no more than about 40%, no more than about 50%, no more than about 60%, no more than about 70%, no more than about 80%, no more than about 90%, or no more than about 100% relative to the baseline insulin measurement. In some embodiments, the insulin measurement is decreased by 2.5%, 5%, 7.5%, 19%, 20%, 30%, 40%, 50%, 60%, 70%, 80%, 90%, 100%, or by a range defined by any of the two aforementioned percentages.

In some embodiments, the measurement is a systolic blood (SBP) pressure measurement. In some embodiments, the SBP measurement is measured in mm of mercury (mm Hg). In some embodiments, the SBP measurement is obtained with a sphygmomanometer. The SBP measurement may be indicative of hypertension. The SBP measurement may be indicative of normal blood pressure. The SBP measurement may include a cerebral SBP measurement.

In some embodiments, the composition reduces the SBP measurement relative to the baseline SBP measurement. In some embodiments, the reduction is measured directly in the subject after administering the composition to the subject. In some embodiments, the SBP measurement is decreased by about 2.5% or more, about 5% or more, or about 7.5% or more, relative to the baseline SBP measurement. In some embodiments, the SBP measurement is decreased by about 10% or more, relative to the baseline SBP measurement. In some embodiments, the SBP measurement is decreased by about 20% or more, about 30% or more, about 40)% or more, about 50% or more, about 60)% or more, about 70% or more, about 80% or more, about 90% or more, relative to the baseline SBP measurement. In some embodiments, the SBP measurement is decreased by no more than about 2.5%, no more than about 5%, or no more than about 7.5%, relative to the baseline SBP measurement. In some embodiments, the SBP measurement is decreased by no more than about 10%, relative to the baseline SBP measurement. In some embodiments, the SBP measurement is decreased by no more than about 20)%, no more than about 30%, no more than about 40%, no more than about 50%, no more than about 60)%, no more than about 70%, no more than about 80%, or no more than about 90% relative to the baseline SBP measurement. In some embodiments, the SBP measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a diastolic blood (DBP) pressure measurement. In some embodiments, the DBP measurement is measured in mm of mercury (mm Hg). In some embodiments, the DBP measurement is obtained with a sphygmomanometer. The DBP measurement may be indicative of hypertension. The DBP measurement may be indicative of normal blood pressure. The DBP measurement may include a cerebral DBP measurement.

In some embodiments, the composition reduces the DBP measurement relative to the baseline DBP measurement. In some embodiments, the reduction is measured directly in the subject after administering the composition to the subject. In some embodiments, the DBP measurement is decreased by about 2.5% or more, about 5% or more, or about 7.5% or more, relative to the baseline DBP measurement. In some embodiments, the DBP measurement is decreased by about 10% or more, relative to the baseline DBP measurement. In some embodiments, the DBP measurement is decreased by about 20% or more, about 30% or more, about 40)% or more, about 50% or more, about 60% or more, about 70)% or more, about 80% or more, about 90% or more, relative to the baseline DBP measurement. In some embodiments, the DBP measurement is decreased by no more than about 2.5%, no more than about 5%, or no more than about 7.5%, relative to the baseline DBP measurement. In some embodiments, the DBP measurement is decreased by no more than about 10%, relative to the baseline DBP measurement. In some embodiments, the DBP measurement is decreased by no more than about 20)%, no more than about 30%, no more than about 40)%, no more than about 50)%, no more than about 60)%, no more than about 70)%, no more than about 80%, or no more than about 90% relative to the baseline DBP measurement. In some embodiments, the DBP measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a systolic heart function measurement. A heart systolic function measurement may include a measure of heart pumping capacity. An example of a systolic function measurement includes an ejection fraction measurement. A ejection fraction measurement may include a left ventricular ejection fraction measurement, a right ventricular ejection fraction measurement, a left atrial ejection fraction measurement, or a right atrial ejection fraction measurement. In some embodiments, the ejection fraction measurement includes a left ventricular ejection fraction measurement. A subject with heart failure, for example, may have a left ventricular ejection fraction below 60%, below 50%, below 40%, below 30%, below: 20%, or below 10%. In some embodiments, a left ventricular ejection fraction below 35% is indicative of systolic dysfunction. Another example of a systolic heart function measurement is a cardiac output measurement. The systolic heart function measurement may be measured using a medical imaging device such as an ultrasound (e.g., an echocardiography device) or magnetic resonance imaging device.

In some embodiments, the composition increases the systolic heart function measurement relative to the baseline systolic heart function measurement. In some embodiments, the increase is measured in a second sample obtained from the subject after administering the composition to the subject. In some embodiments, the systolic heart function measurement is increased by about 2.5% or more, about 5% or more, or about 7.5% or more, relative to the baseline systolic heart function measurement. In some embodiments, the systolic heart function measurement is increased by about 10% or more, relative to the baseline systolic heart function measurement. In some embodiments, the systolic heart function measurement is increased by about 20% or more, about 30% or more, about 40% or more, about 50% or more, about 60% or more, about 70% or more, about 80% or more, about 90% or more, relative to the baseline systolic heart function measurement. In some embodiments, the systolic heart function measurement is increased by about 100% or more, increased by about 250% or more, increased by about 500% or more, increased by about 750% or more, or increased by about 1000% or more, relative to the baseline systolic heart function measurement. In some embodiments, the systolic heart function measurement is increased by no more than about 2.5%, no more than about 5%, or no more than about 7.5%, relative to the baseline systolic heart function measurement. In some embodiments, the systolic heart function measurement is increased by no more than about 10%, relative to the baseline systolic heart function measurement. In some embodiments, the systolic heart function measurement is increased by no more than about 20%, no more than about 30%, no more than about 40%, no more than about 50%, no more than about 60%, no more than about 70%, no more than about 80%, no more than about 90%, or no more than about 100% relative to the baseline systolic heart function measurement. In some embodiments, the systolic heart function measurement is increased by no more than about 100%, increased by no more than about 250%, increased by no more than about 500%, increased by no more than about 750%, or increased by no more than about 1000%, relative to the baseline systolic heart function measurement. In some embodiments, the systolic heart function measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a liver enzyme measurement. In some embodiments, the liver enzyme measurement is an alanine aminotransferase (ALT) measurement. In some embodiments, the liver enzyme measurement is an aspartate aminotransferase (AST) measurement. In some embodiments, the liver enzyme measurement comprises an ALT/AST ratio, or comprises an AST/ALT ratio.

In some embodiments, the measurement is an alanine aminotransferase (ALT) measurement. In some embodiments, the ALT measurement is an ALT concentration (for example. Units/dL). In some embodiments, the ALT measurement is a blood ALT measurement, for example, a blood, serum, or plasma ALT level. In some embodiments, the ALT measurement is obtained by an assay such as an immunoassay, a colorimetric assay, or a fluorescence assay.

In some embodiments, the composition reduces the ALT measurement relative to the baseline ALT measurement. In some embodiments, the reduced ALT is measured in a second blood sample, plasma sample, or serum sample obtained from the subject after administering the composition to the subject. In some embodiments, the ALT measurement is decreased by about 2.5% or more, about 5% or more, or about 7.5% or more, relative to the baseline ALT measurement. In some embodiments, the ALT measurement is decreased by about 10% or more, relative to the baseline ALT measurement. In some embodiments, the ALT measurement is decreased by about 20% or more, about 30% or more, about 40% or more, about 50% or more, about 60% or more, about 70% or more, about 80% or more, about 90% or more, relative to the baseline ALT measurement. In some embodiments, the ALT measurement is decreased by no more than about 2.5%, no more than about 5%, or no more than about 7.5%, relative to the baseline ALT measurement. In some embodiments, the ALT measurement is decreased by no more than about 10%, relative to the baseline ALT measurement. In some embodiments, the ALT measurement is decreased by no more than about 20%, no more than about 30%, no more than about 40%, no more than about 50%, no more than about 60%, no more than about 70%, no more than about 80%, no more than about 90%, or no more than about 100% relative to the baseline ALT measurement. In some embodiments, the ALT measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is an aspartate aminotransferase (AST) measurement. In some embodiments, the AST measurement is an AST concentration (for example. Units/dL). In some embodiments, the AST measurement is a blood AST measurement, for example, a blood, serum, or plasma AST level. In some embodiments, the AST measurement is obtained by an assay such as an immunoassay, a colorimetric assay, or a fluorescence assay.

In some embodiments, the composition reduces the AST measurement relative to the baseline AST measurement. In some embodiments, the reduced AST is measured in a second blood sample, plasma sample, or serum sample obtained from the subject after administering the composition to the subject. In some embodiments, the AST measurement is decreased by about 2.5% or more, about 5% or more, or about 7.5% or more, relative to the baseline AST measurement. In some embodiments, the AST measurement is decreased by about 10% or more, relative to the baseline AST measurement. In some embodiments, the AST measurement is decreased by about 20% or more, about 30% or more, about 40% or more, about 50% or more, about 60% or more, about 70% or more, about 80% or more, about 90% or more, relative to the baseline AST measurement. In some embodiments, the AST measurement is decreased by no more than about 2.5%, no more than about 5%, or no more than about 7.5%, relative to the baseline AST measurement. In some embodiments, the AST measurement is decreased by no more than about 10%, relative to the baseline AST measurement. In some embodiments, the AST measurement is decreased by no more than about 20%, no more than about 30%, no more than about 40%, no more than about 50%, no more than about 60%, no more than about 70%, no more than about 80%, no more than about 90%, or no more than about 100% relative to the baseline AST measurement. In some embodiments, the AST measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a alkaline phosphatase (ALP) measurement. In some embodiments, the ALP measurement is a ALP concentration. In some embodiments, the ALP measurement is a blood ALP measurement. In some embodiments, the ALP measurement is obtained by an assay such as an immunoassay, a colorimetric assay, a chromatography assay, or a fluorescence assay.

In some embodiments, the composition reduces the ALP measurement relative to the baseline ALP measurement. In some embodiments, the reduced ALP is measured in a second blood sample, plasma sample, or serum sample obtained from the subject after administering the composition to the subject. In some embodiments, the ALP measurement is decreased by about 2.5% or more, about 5% or more, or about 7.5% or more, relative to the baseline ALP measurement. In some embodiments, the ALP measurement is decreased by about 10% or more, relative to the baseline ALP measurement. In some embodiments, the ALP measurement is decreased by about 20% or more, about 30% or more, about 40% or more, about 50% or more, about 60)% or more, about 70% or more, about 80% or more, about 90% or more, relative to the baseline ALP measurement. In some embodiments, the ALP measurement is decreased by no more than about 2.5%, no more than about 5%, or no more than about 7.5%, relative to the baseline ALP measurement. In some embodiments, the ALP measurement is decreased by no more than about 10%, relative to the baseline ALP measurement. In some embodiments, the ALP measurement is decreased by no more than about 20%, no more than about 30%, no more than about 40%, no more than about 50%, no more than about 60%, no more than about 70%, no more than about 80%, no more than about 90%, or no more than about 100% relative to the baseline ALP measurement. In some embodiments, the ALP measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a gamma-glutamyl transferase (GGT) measurement. In some embodiments, the GGT measurement is a GGT concentration. In some embodiments, the GGT measurement is a blood GGT measurement. In some embodiments, the GGT measurement is obtained by an assay such as an immunoassay, a colorimetric assay, a chromatography assay, or a fluorescence assay.

In some embodiments, the composition reduces the GGT measurement relative to the baseline GGT measurement. In some embodiments, the reduced GGT is measured in a second blood sample, plasma sample, or serum sample obtained from the subject after administering the composition to the subject. In some embodiments, the GGT measurement is decreased by about 2.5% or more, about 5% or more, or about 7.5% or more, relative to the baseline GGT measurement. In some embodiments, the GGT measurement is decreased by about 10% or more, relative to the baseline GGT measurement. In some embodiments, the GGT measurement is decreased by about 20% or more, about 30% or more, about 40% or more, about 50% or more, about 60% or more, about 70% or more, about 80% or more, about 90% or more, relative to the baseline GGT measurement. In some embodiments, the GGT measurement is decreased by no more than about 2.5%, no more than about 5%, or no more than about 7.5%, relative to the baseline GGT measurement. In some embodiments, the GGT measurement is decreased by no more than about 10%, relative to the baseline GGT measurement. In some embodiments, the GGT measurement is decreased by no more than about 20%, no more than about 30%, no more than about 40%, no more than about 50%, no more than about 60%, no more than about 70%, no more than about 80%, no more than about 90%, or no more than about 100% relative to the baseline GGT measurement. In some embodiments, the GGT measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a liver fibrosis measurement. In some embodiments, the liver fibrosis measurement is a liver fibrosis score (LFS). In some embodiments, the LFS comprises a score of 0, 1, 2, 3, or 4, or a range of scores defined by any two of the aforementioned numbers. In some embodiments, the LFS comprises a score of 0-4. In some embodiments, the LFS is obtained using a scoring system. In some embodiments, the LFS measurement is obtained noninvasively. In some embodiments, the LFS measurement is obtained by a medical imaging device such as a vibration-controlled transient elastography (VCTE) device, a shear wave elastography device, a medical resonance imaging (MRI) device, a magnetic resonance spectroscopy device, a computed tomography device, or an ultrasound device. In some embodiments, the LFS measurement is obtained in a second liver sample. In some embodiments, the LFS is obtained by an assay such as an immunoassay, a colorimetric assay, or a fluorescence assay. In some embodiments, the LFS is obtained using one or more indirect markers or measures of liver fibrosis such as an aspartate aminotransferase-to-platelet ratio index (APRI), a Fibrosis-4 (FIB-4) index, a FibroIndex, a Forns Index, a Hepascore, or a FibroTest. In some embodiments, the LFS is obtained using one or more indirect markers or measures of liver fibrosis such as a FIBROSpect test or a FIBROSpect II test. In some embodiments, the LFS is obtained by RT-qPCR or RNA sequencing of one or more fibrosis-related genes such as a collagen gene. In some embodiments, the LFS or the LFS is obtained using a scoring system upon a visual inspection of a sample such as a histological sample. In some embodiments, the LFS or the LFS is obtained using a stain with an affinity to collagen.

In some embodiments, the composition reduces the LFS relative to the baseline LFS. In some embodiments, the reduced LFS is measured in a second liver sample obtained from the subject after administering the composition to the subject. In some embodiments, the reduced LFS is measured directly in the subject after administering the composition to the subject. In some embodiments, the LFS is decreased by 1 relative to the baseline LFS. In some embodiments, the LFS is decreased by 2 relative to the baseline LFS. In some embodiments, the LFS is decreased by 3 relative to the baseline LFS. In some embodiments, the LFS is decreased by 4 relative to the baseline LFS. In some embodiments, the LFS is decreased by 1 or more, relative to the baseline LFS. In some embodiments, the LFS is decreased by 2 or more, relative to the baseline LFS. In some embodiments, the LFS is decreased by 3 more, relative to the baseline LFS. In some embodiments, the LFS is decreased by no more than 1, relative to the baseline LFS. In some embodiments, the LFS is decreased by no more than 2, relative to the baseline LFS. In some embodiments, the LFS is decreased by no more than 3, relative to the baseline LFS. In some embodiments, the LFS is decreased by no more than 4, relative to the baseline LFS. In some embodiments, the LFS is decreased by 1, 2, 3, or 4, or by a range defined by any of the two aforementioned numbers.

In some embodiments, the liver fibrosis measurement is a nonalcoholic fatty liver disease (NAFLD) fibrosis score. A NAFLD fibrosis score may take into account laboratory test values such as platelet count, albumin, and AST/ALT ratio, and patient characteristics such as BMI, and diabetes status. A NAFLD fibrosis score below −1.455 may be indicative of no fibrosis, mild fibrosis, or moderate fibrosis. A NAFLD fibrosis score between-1.455 and 0.675 may be indicative of severe fibrosis. A NAFLD fibrosis score above 0.675 may be indicative of cirrhosis.

In some embodiments, the composition reduces the NAFLD fibrosis score relative to the baseline NAFLD fibrosis score. In some embodiments, the NAFLD fibrosis score is decreased by about 2.5% or more, about 5% or more, or about 7.5% or more, relative to the baseline NAFLD fibrosis score. In some embodiments, the NAFLD fibrosis score is decreased by about 10% or more, relative to the baseline NAFLD fibrosis score. In some embodiments, the NAFLD fibrosis score is decreased by about 20% or more, about 30% or more, about 40% or more, about 50% or more, about 60% or more, about 70% or more, about 80% or more, about 90% or more, relative to the baseline NAFLD fibrosis score. In some embodiments, the NAFLD fibrosis score is decreased by no more than about 2.5%, no more than about 5%, or no more than about 7.5%, relative to the baseline NAFLD fibrosis score. In some embodiments, the NAFLD fibrosis score is decreased by no more than about 10%, relative to the baseline NAFLD fibrosis score. In some embodiments, the NAFLD fibrosis score is decreased by no more than about 20%, no more than about 30%, no more than about 40%, no more than about 50%, no more than about 60%, no more than about 70%, no more than about 80%, no more than about 90%, or no more than about 100% relative to the baseline NAFLD fibrosis score. In some embodiments, the NAFLD fibrosis score is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a non-alcoholic fatty liver disease (NAFLD) activity score. In some embodiments, the NAFLD activity score comprises a numerical value such as a number of points. In some embodiments, the numerical value is 0, 1, 2, 3, 4, 5, 6, 7, or 8, or a range defined by any two of the aforementioned numerical values. In some embodiments, the numerical value is 0-8. In some embodiments, the NAFLD activity score comprises a steatosis grade such as a liver fat percentage. In some embodiments, a steatosis grade <5% comprises 0 points in the NAFLD activity score. In some embodiments, a steatosis grade of 5-33% comprises 1 point in the NAFLD activity score. In some embodiments, a steatosis grade of 34-66% comprises 2 points in the NAFLD activity score. In some embodiments, a steatosis grade of >66% comprises 3 points in the NAFLD activity score. In some embodiments, the NAFLD activity score comprises a lobular inflammation grade. In some embodiments, the lobular inflammation grade comprises an assessment of inflammatory foci. In some embodiments, a lobular inflammation grade comprising 0 foci comprises 0 points in the NAFLD activity score. In some embodiments, a lobular inflammation grade comprising 1 focus per a field (such as a 20× field or a 200× field) comprises 1 point in the NAFLD activity score. In some embodiments, a lobular inflammation grade comprising 2-4 foci per field comprises 2 points in the NAFLD activity score. In some embodiments, a lobular inflammation grade comprising >4 foci per field comprises 3 points in the NAFLD activity score. In some embodiments, the NAFLD activity score comprises a liver cell injury grade such as an amount of ballooning cells. In some embodiments, a liver cell injury comprising no ballooning cells comprises 0 points in the NAFLD activity score. In some embodiments, a liver cell injury comprising some new balloon cells comprises 1 point in the NAFLD activity score. In some embodiments, a liver cell injury comprising many ballooning cells or prominent ballooning comprises 2 points in the NAFLD activity score. In some embodiments, the NAFLD activity score is obtained invasively, based on histology, and/or in a liver biopsy.

In some embodiments, the composition reduces the NAFLD activity score relative to the baseline NAFLD activity score. In some embodiments, the reduced NAFLD activity score is measured in a second liver sample obtained from the subject after administering the composition to the subject. In some embodiments, the NAFLD activity score is decreased by 1 relative to the baseline NAFLD activity score. In some embodiments, the NAFLD activity score is decreased by 2 relative to the baseline NAFLD activity score. In some embodiments, the NAFLD activity score is decreased by 3 relative to the baseline NAFLD activity score. In some embodiments, the NAFLD activity score is decreased by 4 relative to the baseline NAFLD activity score. In some embodiments, the NAFLD activity score is decreased by 5 relative to the baseline NAFLD activity score. In some embodiments, the NAFLD activity score is decreased by 6 relative to the baseline NAFLD activity score. In some embodiments, the NAFLD activity score is decreased by 7 relative to the baseline NAFLD activity score. In some embodiments, the NAFLD activity score is decreased by 8 relative to the baseline NAFLD activity score. In some embodiments, the NAFLD activity score is decreased by 1 or more, relative to the baseline NAFLD activity score. In some embodiments, the NAFLD activity score is decreased by 2 or more, 3 or more, 4 or more, 5 or more, 6 or more, 7 or more, or 8 or more, relative to the baseline NAFLD activity score. In some embodiments, the NAFLD activity score is decreased by no more than 1, no more than 2, no more than 3, no more than 4, no more than 5, no more than 6, no more than 7, or no more than 8, relative to the baseline NAFLD activity score. In some embodiments, the NAFLD activity score is decreased by 1, 2, 3, 4, 5, 6, 7, or 8, or by a range defined by any of the two aforementioned numbers.

In some embodiments, the measurement is a liver steatosis measurement. In some embodiments, the liver steatosis measurement is a liver fat percentage (LFP) measurement. In some embodiments, the measurement is a LFP measurement. In some embodiments, the LFP measurement is indicated as a mass/mass percentage of fat/total tissue. In some embodiments, the LFP measurement is indicated as a mass/volume percentage of fat/total tissue. In some embodiments, the LFP measurement is indicated as a volume/mass percentage of fat/total tissue. In some embodiments, the LFP measurement is indicated as a volume/volume percentage of fat/total tissue. In some embodiments, the LFP measurement is indicated as a score. In some embodiments, the LFP measurement is obtained noninvasively. In some embodiments, the LFP measurement is obtained by a medical imaging device. In some embodiments, the LFP measurement is obtained by a device such as a medical resonance imaging (MRI) device, a magnetic resonance spectroscopy device, a computed tomography device, a controlled attenuation parameter (CAP), a transient elastography device, or an ultrasound device. In some embodiments, the LFP measurement is obtained in a second liver sample. In some embodiments, the LFP measurement comprises a liver triglyceride measurement. In some embodiments, the LFP measurement is obtained by an assay such as an immunoassay, a colorimetric assay, or a fluorescence assay. In some embodiments, the LFP measurement or the LFP measurement is obtained using a scoring system upon a visual inspection of a sample such as a histological sample. In some embodiments, the LFP measurement or the LFP measurement is obtained using a stain with an affinity to fats, such as a lysochrome diazo dye.

In some embodiments, the composition reduces the LFP measurement relative to the baseline LFP measurement. In some embodiments, the reduced LFP is measured in a second liver sample obtained from the subject after administering the composition to the subject. In some embodiments, the reduced LFP is measured directly in the subject after administering the composition to the subject. In some embodiments, the LFP measurement is decreased by about 2.5% or more, about 5% or more, or about 7.5% or more, relative to the baseline LFP measurement. In some embodiments, the LFP measurement is decreased by about 10% or more, relative to the baseline LFP measurement. In some embodiments, the LFP measurement is decreased by about 20% or more, about 30% or more, about 40% or more, about 50% or more, about 60% or more, about 70% or more, about 80% or more, about 90% or more, relative to the baseline LFP measurement. In some embodiments, the LFP measurement is decreased by no more than about 2.5%, no more than about 5%, or no more than about 7.5%, relative to the baseline LFP measurement. In some embodiments, the LFP measurement is decreased by no more than about 10%, relative to the baseline LFP measurement. In some embodiments, the LFP measurement is decreased by no more than about 20%, no more than about 30%, no more than about 40%, no more than about 50%, no more than about 60%, no more than about 70%, no more than about 80%, no more than about 90%, or no more than about 100% relative to the baseline LFP measurement. In some embodiments, the LFP measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is an PLIN1 protein measurement. In some embodiments, the PLIN1 protein measurement comprises an PLIN1 protein level. In some embodiments, the PLIN1 protein level is indicated as a mass or percentage of PLIN1 protein per sample weight. In some embodiments, the PLIN1 protein level is indicated as a mass or percentage of PLIN1 protein per sample volume. In some embodiments, the PLIN1 protein level is indicated as a mass or percentage of PLIN1 protein per total protein within the sample. In some embodiments, the PLIN1 protein measurement is a circulating PLIN1 protein measurement. In some embodiments, the PLIN1 protein measurement is obtained by an assay such as an immunoassay, a colorimetric assay, or a fluorescence assay.

In some embodiments, the composition reduces the PLIN1 protein measurement relative to the baseline PLIN1 protein measurement. In some embodiments, the composition reduces circulating PLIN1 protein levels relative to the baseline PLIN1 protein measurement. In some embodiments, the composition reduces tissue PLIN1 protein levels relative to the baseline PLIN1 protein measurement. In some embodiments, the reduced PLIN1 protein levels are measured in a second sample obtained from the subject after administering the composition to the subject. In some embodiments, the PLIN1 protein measurement is decreased by about 2.5% or more, about 5% or more, or about 7.5% or more, relative to the baseline PLIN1 protein measurement. In some embodiments, the PLIN1 protein measurement is decreased by about 10% or more, relative to the baseline PLIN1 protein measurement. In some embodiments, the PLIN1 protein measurement is decreased by about 20% or more, about 30% or more, about 40)% or more, about 50)% or more, about 60% or more, about 70% or more, about 80% or more, about 90% or more, or about 100%, relative to the baseline PLIN1 protein measurement. In some embodiments, the PLIN1 protein measurement is decreased by no more than about 2.5%, no more than about 5%, or no more than about 7.5%, relative to the baseline PLIN1 protein measurement. In some embodiments, the PLIN1 protein measurement is decreased by no more than about 10%, relative to the baseline PLIN1 protein measurement. In some embodiments, the PLIN1 protein measurement is decreased by no more than about 20%, no more than about 30%, no more than about 40)%, no more than about 50%, no more than about 60%, no more than about 70%, no more than about 80%, no more than about 90%, or no more than about 100% relative to the baseline PLIN1 protein measurement. In some embodiments, the PLIN1 protein measurement is decreased by 2.5%, 5%, 7.5%, 19%, 20%, 30%, 40%, 50%, 60%, 70%, 80%, 90%, or 100%, or by a range defined by any of the two aforementioned percentages.

In some embodiments, the measurement is an PLIN1 mRNA measurement. In some embodiments, the PLIN1 mRNA measurement comprises an PLIN1 mRNA level. In some embodiments, the PLIN1 mRNA level is indicated as an amount or percentage of PLIN1 mRNA per sample weight. In some embodiments, the PLIN1 mRNA level is indicated as an amount or percentage of PLIN1 mRNA per sample volume. In some embodiments, the PLIN1 mRNA level is indicated as an amount or percentage of PLIN1 mRNA per total mRNA within the sample. In some embodiments, the PLIN1 mRNA level is indicated as an amount or percentage of PLIN1 mRNA per total nucleic acids within the sample. In some embodiments, the PLIN1 mRNA level is indicated relative to another mRNA level, such as an mRNA level of a housekeeping gene, within the sample. In some embodiments, the PLIN1 mRNA measurement is obtained by an assay such as a PCR assay. In some embodiments, the PCR comprises qPCR. In some embodiments, the PCR comprises reverse transcription of the PLIN1 mRNA.

In some embodiments, the composition reduces the PLIN1 mRNA measurement relative to the baseline PLIN1 mRNA measurement. In some embodiments, the PLIN1 mRNA measurement is obtained in a second sample obtained from the subject after administering the composition to the subject. In some embodiments, the composition reduces PLIN1 mRNA levels relative to the baseline PLIN1 mRNA levels. In some embodiments, the reduced PLIN1 mRNA levels are measured in a second sample obtained from the subject after administering the composition to the subject. In some embodiments, the second sample is a liver sample. In some embodiments, the second sample is an adipose sample. In some embodiments, the PLIN1 mRNA measurement is reduced by about 2.5% or more, about 5% or more, or about 7.5% or more, relative to the baseline PLIN1 mRNA measurement. In some embodiments, the PLIN1 mRNA measurement is decreased by about 10% or more, relative to the baseline PLIN1 mRNA measurement. In some embodiments, the PLIN1 mRNA measurement is decreased by about 20% or more, about 30% or more, about 40% or more, about 50% or more, about 60% or more, about 70% or more, about 80% or more, about 90% or more, or about 100%, relative to the baseline PLIN1 mRNA measurement. In some embodiments, the PLIN1 mRNA measurement is decreased by no more than about 2.5%, no more than about 5%, or no more than about 7.5%, relative to the baseline PLIN1 mRNA measurement. In some embodiments, the PLIN1 mRNA measurement is decreased by no more than about 10%, relative to the baseline PLIN1 mRNA measurement. In some embodiments, the PLIN1 mRNA measurement is decreased by no more than about 20%, no more than about 30%, no more than about 40%, no more than about 50%, no more than about 60%, no more than about 70%, no more than about 80%, no more than about 90%, or no more than about 100%, relative to the baseline PLIN1 mRNA measurement. In some embodiments, the PLIN1 mRNA measurement is decreased by 2.5%, 5%, 7.5%, 10%, 20%, 30%, 40%, 50%, 60%, 70%, 80%, 90%, or 100% or by a range defined by any of the two aforementioned percentages.

### III. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”. “an” and “the” include plural references unless the context clearly dictates otherwise. For example, the term “a sample” includes a plurality of samples, including mixtures thereof.

The terms “determining.” “measuring.” “evaluating.” “assessing.” “assaying.” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” and “patient” may be used interchangeably herein. A “subject” can be a biological entity containing expressed genetic materials. The biological entity can be a plant, animal, or microorganism, including, for example, bacteria, viruses, fungi, and protozoa. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

“Treatment” or “treating” may include an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treatment via administration of a compound described herein does not require the involvement of a medical professional.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons.

The terms “C_{x-y} alkenyl” and “C_{x-y} alkynyl” refer to substituted or unsubstituted unsaturated aliphatic groups analogous in length and possible substitution to the alkyls described above, but that contain at least one double or triple bond, respectively.

The term “carbocycle” as used herein refers to a saturated, unsaturated or aromatic ring in which each atom of the ring is carbon. Carbocycle includes 3- to 10-membered monocyclic rings, 5- to 12-membered bicyclic rings, 5- to 12-membered spiro bicycles, and 5- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle further includes spiro bicyclic rings such as spiropentane. A bicyclic carbocycle includes any combination of ring sizes such as 3-3 spiro ring systems, 4-4 spiro ring systems, 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, naphthyl, and bicyclo[1.1.1]pentanyl.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

The term “cycloalkyl” refers to a saturated ring in which each atom of the ring is carbon. Cycloalkyl may include monocyclic and polycyclic rings such as 3- to 10-membered monocyclic rings, 5- to 12-membered bicyclic rings, 5- to 12-membered spiro bicycles, and 5- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, spiropentane, norbomyl (i.e., bicyclo[2.2.1]heptanyl), decalinyl, 7,7 dimethyl bicyclo[2.2.1]heptanyl, bicyclo[1.1.1]pentanyl, and the like.

The term “cycloalkenyl” refers to a saturated ring in which each atom of the ring is carbon and there is at least one double bond between two ring carbons. Cycloalkenyl may include monocyclic and polycyclic rings such as 3- to 10-membered monocyclic rings, 6- to 12-membered bicyclic rings, and 5- to 12-membered bridged rings. In other embodiments, a cycloalkenyl comprises five to seven carbon atoms. The cycloalkenyl may be attached to the rest of the molecule by a single bond. Examples of monocyclic cycloalkenyls include, e.g., cyclopentenyl, cyclohexenyl, cycloheptenyl, and cyclooctenyl.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally further substituted as described herein.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, 5- to 12-membered spiro bicycles, and 5- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. A bicyclic heterocycle further includes spiro bicyclic rings, e.g., 5 to 12-membered spiro bicycles, such as 2-oxa-6-azaspiro[3.3]heptane.

The term “heteroaryl” refers to a radical derived from a 5 to 18 membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is aromatic, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzoxazolyl, benzo[d]thiazolyl, benzothiadiazolyl, benzo[b][1,4]dioxepinyl, benzo[b][1,4]oxavinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl. 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridavinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridavinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridavinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl).

The term “heterocycloalkyl” refers to a saturated ring with carbon atoms and at least one heteroatom. Exemplary heteroatoms include N, O, Si, P, B, and S atoms. Heterocycloalkyl may include monocyclic and polycyclic rings such as 3- to 10-membered monocyclic rings, 6- to 12-membered bicyclic rings, 5- to 12-membered spiro bicycles, and 5- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 2-oxa-6-azaspiro[3.3]heptane, and 1,1-dioxo-thiomorpholinyl.

The term “heterocycloalkenyl” refers to an unsaturated ring with carbon atoms and at least one heteroatom and there is at least one double bond between two ring carbons. Heterocycloalkenyl does not include heteroaryl rings. Exemplary heteroatoms include N, O, Si, P, B, and S atoms. Heterocycloalkenyl may include monocyclic and polycyclic rings such as 3- to 10-membered monocyclic rings, 6- to 12-membered bicyclic rings, and 5- to 12-membered bridged rings. In other embodiments, a heterocycloalkenyl comprises five to seven ring atoms. The heterocycloalkenyl may be attached to the rest of the molecule by a single bond. Examples of monocyclic cycloalkenyls include, e.g., pyrroline (dihydropyrrole), pyrazoline (dihydropyrazole), imidazoline (dihydroimidazole), triazoline (dihydrothiazole), dihydrofuran, dihydrothiophene, oxazoline (dihydrooxazole), isoxazoline (dihydroisoxazole), thiazoline (dihydrothiazole), isothiazoline (dihydroisothiazole), oxadiazoline (dihydroxadiazole), thiadiazoline (dihydrothiadiazole), dihydropyridine, tetrahydropyridine, dihydropyridazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a}), —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O),N(R^{a}) (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a}), —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O),N(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a} valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a}), —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)R^{a} (where t is 1 or 2), —R^{b}—S(O)ₜOR^{a} (where t is 1 or 2) and —R^{b}—S(O)ₜN(R^{a}) (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

Double bonds to oxygen atoms, such as oxo groups, are represented herein as both “═O” and “(O)”. Double bonds to nitrogen atoms are represented as both “═NR” and “(NR)”. Double bonds to sulfur atoms are represented as both “═S” and “(S)”.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soy bean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

Some embodiments refer to nucleic acid sequence information. It is contemplated that in some embodiments, thymine (T) may be interchanged with uracil (U), or vice versa. For example, some sequences in the sequence listing may recite Ts, but these may be replaced with Us in some embodiments. In some oligonucleotides with nucleic acid sequences that include uracil, the uracil may be replaced with thymine. Similarly, in some oligonucleotides with nucleic acid sequences that include thymine, the thymine may be replaced with uracil. In some embodiments, an oligonucleotide such as an siRNA comprises or consists of RNA. In some embodiments, the oligonucleotide may include DNA. For example, the oligonucleotide may include 2′ deoxyribonucleotides. An ASO may comprise or consist of DNA.

The section headings used herein are for organizational purposes only and are not to be construed as limiting the subject matter described.

### VI. EXAMPLES

### Example 1: Loss-of-Function Variants in PLIN1 Demonstrate Protective Associations for Lipid Traits and Cardiometabolic Disease

Variants in PLIN1 were evaluated for associations with blood lipids, cardiometabolic disease and related traits in approximately 452.000 individuals with genotype data from the UK Biobank cohort. Variants evaluated included (1) rs139271800, a rare (AAF=0.001) missense variant (Lue90)Pro; L90P), (2) rs750619494, a rare (AAF=0.0003) frameshift variant (Thr338 AspfsTer51; T338 DfsTer51), (3) rs150822845, a rare (AAF=0.0001) stop-gained variant (Arg93Ter; R93Ter) and (4) rs201579932, a rare (AAF=0.0003) splice acceptor variant. The four variants were considered to be hypomorphic or loss of function variants that may result in a decrease in the abundance or activity of the PLIN1 gene product. Stepwise conditional analyses in multiple traits, as well as direct evaluation of linkage disequilibrium, confirmed that they are independent variants. A PLIN1 loss of function gene burden test was also evaluated which aggregated carriers of rare annotated PLIN1 loss of function variants to increase statistical power.

The analyses resulted in identification of associations for the individual PLIN1 variants and for the PLIN1 burden test. For example, there were protective associations with multiple cardiometabolic traits. PLIN1 variants were individually or collectively associated with decreased triglycerides, increased HDL, decreased LDL and decreased APOB (Table 3A and 3B).

TABLE 3A
PLIN1 lipid associations

| | | | | Triglycerides | | HDL Cholesterol | |
|---|---|---|---|---|---|---|---|
| | | | | (n = 430,833) | | (n = 396,317) | |
| | | | | P | | P | |
| Variant | Gene | Function | AAF | value | Beta | value | Beta |
| rs139271800 | PLIN1 | Missense; | 0.001 | 2.43E−08 | ↓−0.035 | 6.39E−09 | ↑0.019 |
| | | L90P | | | | | |
| rs750619494 | PLIN1 | Frameshift; | 0.0003 | 3.40E−07 | ↓−0.068 | 3.10E−10 | ↑0.042 |
| | | T338D(fsTer51) | | | | | |
| rs150822845 | PLIN1 | Stop gained; | 0.0001 | 1.26E−03 | ↓−0.063 | 1.69E−06 | ↑0.048 |
| | | R93Ter | | | | | |
| rs201579932 | PLIN1 | Splice donor; | 0.0003 | 5.33E−03 | ↓−0.035 | 6.57E−03 | ↑0.017 |
| | | 964-1G > C | | | | | |
| LoF Burden | PLIN1 | Loss of | 0.0009 | 2.90E−10 | ↓−0.048 | 1.66E−15 | ↑0.031 |
| | | Function | | | | | |
| | | Burden | | | | | |

TABLE 3B
PLIN1 lipid associations

| | |
|---|---|
| LDL Cholesterol | APOB |
| (n = 431,767) | (n = 430,494) |

| Variant | P value | Beta | P value | Beta |
|---|---|---|---|---|
| rs139271800 | 0.038 | ↓-0.007 | 0.029 | ↓-0.007 |
| rs750619494 | 0.050 | ↓-0.014 | 0.013 | ↓-0.016 |
| rs150822845 | 0.373 | ↓-0.009 | 0.360 | ↓-0.009 |
| rs201579932 | 0.466 | ↓-0.005 | 0.701 | ↓-0.002 |
| LoF Burden | 0.048 | ↓-0.008 | 0.066 | ↓-0.007 |

The PLIN1 loss of function gene burden was additionally associated with decreased risk of myocardial infarction, angina, cerebrovascular disease, peripheral vascular disease and hypertension (Table 3C and 3D).

TABLE 3C
PLIN1 cardiovascular associations

| | | | | Myocardial | | | |
|---|---|---|---|---|---|---|---|
| | | | | Infarction | | Angina | |
| | | | | (n = 20,851) | | (n = 23,116) | |
| | | | | P | | P | |
| Variant | Gene | Function | AAF | value | OR | value | OR |
| LoF | PLIN1 | Loss of | 0.0009 | 0.016 | ↓0.601 | 0.021 | ↓0.654 |
| Burden | | Function | | | | | |
| | | Burden | | | | | |

TABLE 3D
PLIN1 cardiovascular associations

| | | | Peripheral | | | |
|---|---|---|---|---|---|---|
| | Cerebrovascular | | Vascular | | | |
| | Disease | | Disease | | Hypertension | |
| | (n = 29,617) | | (n = 11,752) | | (n = 177,488) | |
| | P | | P | | P | |
| Variant | value | OR | value | OR | value | OR |
| LoF | 0.018 | ↓0.704 | 6.56E−03 | ↓0.523 | 2.09E−03 | ↓0.740 |
| Burden | | | | | | |

The PLIN1 loss of function gene burden was additionally associated with decreased blood Aspartate Aminotransferase (AST) and decreased risk of type 2 diabetes (Table 3E).

TABLE 3E
PLIN1 liver and diabetes associations

| | | | | Aspartate | | Type 2 | |
|---|---|---|---|---|---|---|---|
| | | | | Aminotransferase | | Diabetes | |
| | | | | (n = 429,246) | | (n = 27,897) | |
| | | | | P | | P | |
| Variant | Gene | Function | AAF | value | Beta | value | OR |
| LoF | PLIN1 | Loss of | 0.0009 | 0.049 | ↓−0.008 | 4.43E−03 | ↓0.788 |
| Burden | | Function | | | | | |
| | | Burden | | | | | |

These results indicate that loss-of-function of PLIN1 resulted in decreased triglycerides, increased HDL, decreased LDL, decreased APOB and decreased risk of cardiovascular diseases, cerebrovascular diseases and hypertensive diseases, liver diseases and diabetes. These results further indicate that therapeutic inhibition of PLIN1 may result in similar disease-protective effects.

### Example 2: Protective Variants in PLIN1 Result in Less or Truncated PLIN1 Protein

Protein-coding sequence (CDS) expression constructs encoding for wild type. R93Ter and T338D(fsTer51) proteins were generated. The CDS of the protein coding transcript (ENST00000300055) of PLIN1 was cloned into a pcDNA3.1(+) vector driven by a CMV promoter. Empty vector was used as control. For R93Ter (rs150822845) expression constructs, the A allele replaced the G allele at DNA sequence position chr 15:89671538 (human genome build 38). This created an R93Ter premature stop codon. For T338D(fsTer51) (rs750619494) expression constructs, the C allele replaced the CTTCTGCAGGGT allele at DNA position chr15:89667122 (human genome build 38). This created an T338D frameshift resulting in a premature stop codon.

Transfections of COS-7 cells were optimized. COS-7 cells were plated in a T75 flask in complete growth media and grown for 48 hours followed by a media change. Cells were then transfected with 15 μg of plasmid DNA and 19 μl of TransIT-2020. Cells were incubated for 48 hours, and then harvested.

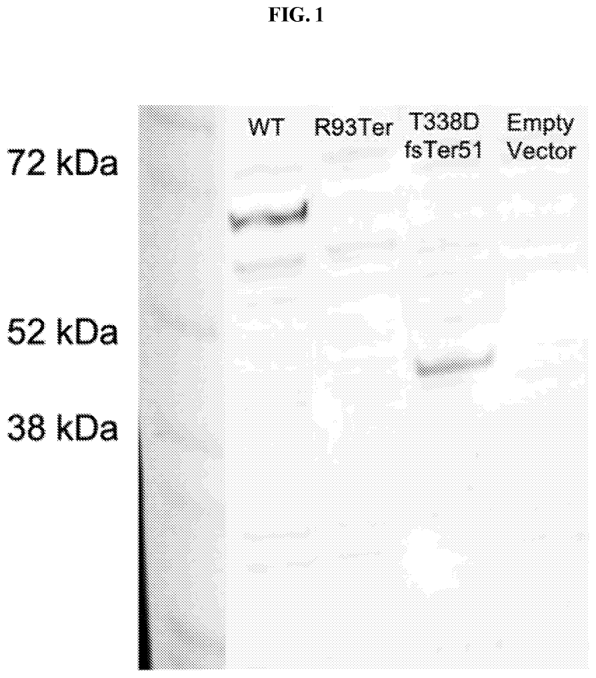

Cell lysates from transfected cells were assayed to evaluate intracellular PLIN1 protein by western blot (FIG. 1). In empty vector transfected COS-7 cells. PLIN1 was not detectable by western blot. In cells transfected with the wild type construct. PLIN1 was detected by western blot as a band between 52-72 kDa. In cells transfected with the R93Ter construct. PLIN1 was not detectable by western blot, suggesting the premature stop codon results in loss of PLIN1 via nonsense mediated decay or degradation at the protein level. In cells transfected with the T338D(fsTer51) construct, a truncated protein product was detected by western Blot as a band ˜40 kDa, near the predicted size of the T338D(fsTer51) truncated product.

These data provide experimental verification that PLIN1 gene variants associated with decreased triglycerides, increased HDL, decreased LDL, decreased APOB and decreased risk of cardiovascular diseases, cerebrovascular diseases and hypertensive diseases, liver diseases and diabetes, resulted in loss of PLIN1 protein abundance or function. Accordingly, in some cases therapeutic inhibition or modulation of PLIN1 may be an effective genetically-informed method of treatment for these measures and diseases.

### Example 3: Bioinformatic Selection of Sequences in Order to Identify Therapeutic siRNAs to Downmodulate Expression of the PLIN1 mRNA

Screening sets were defined based on bioinformatic analysis. Therapeutic siRNAs were designed to target human PLIN1, and the PLIN1 sequence of at least one toxicology-relevant species, in this case, the non-human primates (NHP) rhesus and cynomolgus monkeys. Drivers for the design of the screening set were predicted specificity of the siRNAs against the transcriptome of the relevant species as well as cross-reactivity between species. Predicted specificity in human, rhesus monkey, cynomolgus monkey, mouse and rat was determined for sense (S) and antisense (AS) strands. These were assigned a “specificity score” which considers the likelihood of unintended downregulation of any other transcript by full or partial complementarity of an siRNA strand (up to 4 mismatches within positions 2-18) as well as the number and positions of mismatches. Thus, off-target(s) for antisense and sense strands of each siRNA were identified. In addition, the number of potential off-targets was used as an additional specificity factor in the specificity score. As identified, siRNAs with high specificity and a low number of predicted off-targets provide a benefit of increased targeting specificity.

In addition to selecting siRNA sequences with high sequence specificity to PLIN1 mRNA, siRNA sequences within the seed region were analyzed for similarity to seed regions of known miRNAs, siRNAs can function in a miRNA like manner via base-pairing with complementary sequences within the 3′-UTR of mRNA molecules. The complementarity typically encompasses the 5′-bases at positions 2-7 of the miRNA (seed region). To circumvent siRNAs to act via functional miRNA binding sites, siRNA strands containing natural miRNA seed regions were avoided. Seed regions identified in miRNAs from human, mouse, rat, rhesus monkey, dog, rabbit and pig are referred to as “conserved”. Combining the “specificity score” with miRNA seed analysis yielded a “specificity category”. This is divided into categories 1-4, with 1 having the highest specificity and 4 having the lowest specificity. Each strand of the siRNA is assigned to a specificity category.

Specificity and species cross-reactivity was assessed for human, cynomolgus monkey, rhesus monkey, mouse and rat PLIN1. The analysis was based on a canonical siRNA design using 19 bases and 17 bases (without considering positions 1 and 19) for cross-reactivity. Full match as well as single mismatch analyses were included.

Analysis of the human Single Nucleotide Polymorphism (SNP) database (NCBI-DB-SNP) to identify siRNAs targeting regions with known SNPs was also carried out to identify siRNAs that may be non-functional in individuals containing the SNP. Information regarding the positions of SNPs within the target sequence as well as minor allele frequency (MAF) in case data was obtained in this analysis.

Initial analysis of the relevant PLIN1 mRNA sequence revealed few sequences that fulfil the specificity parameters and at the same time target PLIN1 mRNA in all of the analyzed relevant species. Therefore, it was decided to design independent screening subsets for the therapeutic siRNAs.

The siRNAs in these subsets recognize the human, cynomolgus monkey, rhesus monkey PLIN1 sequences. Therefore, the siRNAs in these subsets can be used to target human PLIN1 in a therapeutic setting.

The number of siRNA sequences that can be derived from human PLIN1 mRNA (ENST00000300055.10, SEQ ID NO: 6014) without consideration of specificity or species cross-reactivity was 2898 (sense and antisense strand sequences included in SEQ ID NOS: 1-5796).

Prioritizing sequences for target specificity, species cross-reactivity, miRNA seed region sequences and SNPs as described above yields subset A. Subset A contains 145 siRNAs whose base sequences are shown in Table 4.

TABLE 4
Sequences in siRNA subset A

| SEQ ID | sense strand sequence | SEQ ID | antisense strand sequence |
|---|---|---|---|
| NO: | (5′-3′) | NO: | (5′-3′) |
| 209 | GCUGCCGGUGGUGAGUGGC | 3107 | GCCACUCACCACCGGCAGC |
| 213 | CCGGUGGUGAGUGGCACCU | 3111 | AGGUGCCACUCACCACCGG |
| 218 | GGUGAGUGGCACCUGCGAA | 3116 | UUCGCAGGUGCCACUCACC |
| 300 | GCCUAUGAGAAGGGCGUGC | 3198 | GCACGCCCUUCUCAUAGGC |
| 301 | CCUAUGAGAAGGGCGUGCA | 3199 | UGCACGCCCUUCUCAUAGG |
| 302 | CUAUGAGAAGGGCGUGCAG | 3200 | CUGCACGCCCUUCUCAUAG |
| 310 | AGGGCGUGCAGAGCGCCAG | 3208 | CUGGCGCUCUGCACGCCCU |
| 311 | GGGCGUGCAGAGCGCCAGU | 3209 | ACUGGCGCUCUGCACGCCC |
| 313 | GCGUGCAGAGCGCCAGUAG | 3211 | CUACUGGCGCUCUGCACGC |
| 341 | CUGGAGCAUGGAGCCGGUG | 3239 | CACCGGCUCCAUGCUCCAG |
| 344 | GAGCAUGGAGCCGGUGGUC | 3242 | GACCACCGGCUCCAUGCUC |
| 378 | CAGUUCACAGCUGCCAAUG | 3276 | CAUUGGCAGCUGUGAACUG |
| 393 | AAUGAGCUGGCCUGCCGAG | 3291 | CUCGGCAGGCCAGCUCAUU |
| 394 | AUGAGCUGGCCUGCCGAGG | 3292 | CCUCGGCAGGCCAGCUCAU |
| 399 | CUGGCCUGCCGAGGCUUGG | 3297 | CCAAGCCUCGGCAGGCCAG |
| 404 | CUGCCGAGGCUUGGACCAC | 3302 | GUGGUCCAAGCCUCGGCAG |
| 405 | UGCCGAGGCUUGGACCACC | 3303 | GGUGGUCCAAGCCUCGGCA |
| 407 | CCGAGGCUUGGACCACCUG | 3305 | CAGGUGGUCCAAGCCUCGG |
| 501 | CUCCGCAGUGCCAGAAACA | 3399 | UGUUUCUGGCACUGCGGAG |
| 502 | UCCGCAGUGCCAGAAACAG | 3400 | CUGUUUCUGGCACUGCGGA |
| 503 | CCGCAGUGCCAGAAACAGC | 3401 | GCUGUUUCUGGCACUGCGG |
| 530 | UCCCAUCGCGAGCACUUCA | 3428 | UGAAGUGCUCGCGAUGGGA |
| 533 | CAUCGCGAGCACUUCAGAC | 3431 | GUCUGAAGUGCUCGCGAUG |
| 534 | AUCGCGAGCACUUCAGACA | 3432 | UGUCUGAAGUGCUCGCGAU |
| 540 | AGCACUUCAGACAAGGUCC | 3438 | GGACCUUGUCUGAAGUGCU |
| 541 | GCACUUCAGACAAGGUCCU | 3439 | AGGACCUUGUCUGAAGUGC |
| 601 | CCAGAGACACUGCGGAAUU | 3499 | AAUUCCGCAGUGUCUCUGG |
| 606 | GACACUGCGGAAUUUGCUG | 3504 | CAGCAAAUUCCGCAGUGUC |
| 610 | CUGCGGAAUUUGCUGCCAA | 3508 | UUGGCAGCAAAUUCCGCAG |
| 616 | AAUUUGCUGCCAACACUCG | 3514 | CGAGUGUUGGCAGCAAAUU |
| 617 | AUUUGCUGCCAACACUCGA | 3515 | UCGAGUGUUGGCAGCAAAU |
| 618 | UUUGCUGCCAACACUCGAG | 3516 | CUCGAGUGUUGGCAGCAAA |
| 619 | UUGCUGCCAACACUCGAGC | 3517 | GCUCGAGUGUUGGCAGCAA |
| 620 | UGCUGCCAACACUCGAGCU | 3518 | AGCUCGAGUGUUGGCAGCA |
| 621 | GCUGCCAACACUCGAGCUG | 3519 | CAGCUCGAGUGUUGGCAGC |
| 622 | CUGCCAACACUCGAGCUGG | 3520 | CCAGCUCGAGUGUUGGCAG |
| 623 | UGCCAACACUCGAGCUGGC | 3521 | GCCAGCUCGAGUGUUGGCA |
| 624 | GCCAACACUCGAGCUGGCC | 3522 | GGCCAGCUCGAGUGUUGGC |
| 625 | CCAACACUCGAGCUGGCCG | 3523 | CGGCCAGCUCGAGUGUUGG |
| 627 | AACACUCGAGCUGGCCGAC | 3525 | GUCGGCCAGCUCGAGUGUU |
| 628 | ACACUCGAGCUGGCCGACU | 3526 | AGUCGGCCAGCUCGAGUGU |
| 629 | CACUCGAGCUGGCCGACUG | 3527 | CAGUCGGCCAGCUCGAGUG |
| 630 | ACUCGAGCUGGCCGACUGG | 3528 | CCAGUCGGCCAGCUCGAGU |
| 631 | CUCGAGCUGGCCGACUGGC | 3529 | GCCAGUCGGCCAGCUCGAG |
| 634 | GAGCUGGCCGACUGGCUUC | 3532 | GAAGCCAGUCGGCCAGCUC |
| 636 | GCUGGCCGACUGGCUUCUG | 3534 | CAGAAGCCAGUCGGCCAGC |
| 649 | CUUCUGGAGGGGCCGACUU | 3547 | AAGUCGGCCCCUCCAGAAG |
| 650 | UUCUGGAGGGGCCGACUUG | 3548 | CAAGUCGGCCCCUCCAGAA |
| 690 | GUGGUGGAGUACCUCCUCC | 3588 | GGAGGAGGUACUCCACCAC |
| 692 | GGUGGAGUACCUCCUCCCU | 3590 | AGGGAGGAGGUACUCCACC |
| 797 | UGGGGCUCUGACCAACACC | 3695 | GGUGUUGGUCAGAGCCCCA |
| 798 | GGGGCUCUGACCAACACCC | 3696 | GGGUGUUGGUCAGAGCCCC |
| 935 | CAUGCAGGCGGUGUCCCGG | 3833 | CCGGGACACCGCCUGCAUG |
| 943 | CGGUGUCCCGGCGGAGGAG | 3841 | CUCCUCCGCCGGGACACCG |
| 1175 | GGCUGUGACAUGGGCACCU | 4073 | AGGUGCCCAUGUCACAGCC |
| 1349 | GCUGUCGCUGAUGGAGCCC | 4247 | GGGCUCCAUCAGCGACAGC |
| 1350 | CUGUCGCUGAUGGAGCCCG | 4248 | CGGGCUCCAUCAGCGACAG |
| 1351 | UGUCGCUGAUGGAGCCCGA | 4249 | UCGGGCUCCAUCAGCGACA |
| 1379 | CCGGGACAUCGACAACCCA | 4277 | UGGGUUGUCGAUGUCCCGG |
| 1400 | AGCCGAGGUCGAGCGCCGG | 4298 | CCGGCGCUCGACCUCGGCU |
| 1401 | GCCGAGGUCGAGCGCCGGG | 4299 | CCCGGCGCUCGACCUCGGC |
| 1404 | GAGGUCGAGCGCCGGGAGG | 4302 | CCUCCCGGCGCUCGACCUC |
| 1551 | GUCGCCACGCCCGCAGCGC | 4449 | GCGCUGCGGGCGUGGCGAC |
| 1589 | CGUGCCCCGCGAGAAGCCA | 4487 | UGGCUUCUCGCGGGGCACG |
| 1607 | AAAGCGCAGGGUCAGCGAC | 4505 | GUCGCUGACCCUGCGCUUU |
| 1608 | AAGCGCAGGGUCAGCGACA | 4506 | UGUCGCUGACCCUGCGCUU |
| 1609 | AGCGCAGGGUCAGCGACAG | 4507 | CUGUCGCUGACCCUGCGCU |
| 1612 | GCAGGGUCAGCGACAGCUU | 4510 | AAGCUGUCGCUGACCCUGC |
| 1613 | CAGGGUCAGCGACAGCUUC | 4511 | GAAGCUGUCGCUGACCCUG |
| 1614 | AGGGUCAGCGACAGCUUCU | 4512 | AGAAGCUGUCGCUGACCCU |
| 1619 | CAGCGACAGCUUCUUCCGG | 4517 | CCGGAAGAAGCUGUCGCUG |
| 1620 | AGCGACAGCUUCUUCCGGC | 4518 | GCCGGAAGAAGCUGUCGCU |
| 1630 | UCUUCCGGCCCAGCGUCAU | 4528 | AUGACGCUGGGCCGGAAGA |
| 1631 | CUUCCGGCCCAGCGUCAUG | 4529 | CAUGACGCUGGGCCGGAAG |
| 1632 | UUCCGGCCCAGCGUCAUGG | 4530 | CCAUGACGCUGGGCCGGAA |
| 1687 | GCAAGAAGAGCUGAGUCGC | 4585 | GCGACUCAGCUCUUCUUGC |
| 1692 | AAGAGCUGAGUCGCCGCAC | 4590 | GUGCGGCGACUCAGCUCUU |
| 1905 | AGCCAGUUUUUAAGGGACA | 4803 | UGUCCCUUAAAAACUGGCU |
| 1907 | CCAGUUUUUAAGGGACACC | 4805 | GGUGUCCCUUAAAAACUGG |
| 1908 | CAGUUUUUAAGGGACACCA | 4806 | UGGUGUCCCUUAAAAACUG |
| 1909 | AGUUUUUAAGGGACACCAG | 4807 | CUGGUGUCCCUUAAAAACU |
| 1911 | UUUUUAAGGGACACCAGAG | 4809 | CUCUGGUGUCCCUUAAAAA |
| 1913 | UUUAAGGGACACCAGAGCC | 4811 | GGCUCUGGUGUCCCUUAAA |
| 1929 | GCCGCAGCCUGCUCUGAUU | 4827 | AAUCAGAGCAGGCUGCGGC |
| 1930 | CCGCAGCCUGCUCUGAUUC | 4828 | GAAUCAGAGCAGGCUGCGG |
| 1934 | AGCCUGCUCUGAUUCUAUG | 4832 | CAUAGAAUCAGAGCAGGCU |
| 1968 | UAAGAGUAAUUGCCUAACU | 4866 | AGUUAGGCAAUUACUCUUA |
| 1969 | AAGAGUAAUUGCCUAACUU | 4867 | AAGUUAGGCAAUUACUCUU |
| 1973 | GUAAUUGCCUAACUUGAUU | 4871 | AAUCAAGUUAGGCAAUUAC |
| 1974 | UAAUUGCCUAACUUGAUUU | 4872 | AAAUCAAGUUAGGCAAUUA |
| 2002 | UUAACCAAACUUGUGGCCA | 4900 | UGGCCACAAGUUUGGUUAA |
| 2076 | CCACGAAUGAGUAACUCCU | 4974 | AGGAGUUACUCAUUCGUGG |
| 2080 | GAAUGAGUAACUCCUGUCA | 4978 | UGACAGGAGUUACUCAUUC |
| 2124 | UUUUGACACAUUCUUAGCA | 5022 | UGCUAAGAAUGUGUCAAAA |
| 2125 | UUUGACACAUUCUUAGCAC | 5023 | GUGCUAAGAAUGUGUCAAA |
| 2126 | UUGACACAUUCUUAGCACU | 5024 | AGUGCUAAGAAUGUGUCAA |
| 2129 | ACACAUUCUUAGCACUGAA | 5027 | UUCAGUGCUAAGAAUGUGU |
| 2131 | ACAUUCUUAGCACUGAACU | 5029 | AGUUCAGUGCUAAGAAUGU |
| 2218 | GCUACUUUGAAGGGAACAA | 5116 | UUGUUCCCUUCAAAGUAGC |
| 2275 | AGUGUCACUUUCUGAGUAC | 5173 | GUACUCAGAAAGUGACACU |
| 2276 | GUGUCACUUUCUGAGUACC | 5174 | GGUACUCAGAAAGUGACAC |
| 2385 | UCACCUGCAUAGUCACUCU | 5283 | AGAGUGACUAUGCAGGUGA |
| 2386 | CACCUGCAUAGUCACUCUU | 5284 | AAGAGUGACUAUGCAGGUG |
| 2390 | UGCAUAGUCACUCUUUUGA | 5288 | UCAAAAGAGUGACUAUGCA |
| 2394 | UAGUCACUCUUUUGAUGCU | 5292 | AGCAUCAAAAGAGUGACUA |
| 2448 | GUAUAGCCACAGUUCAUCC | 5346 | GGAUGAACUGUGGCUAUAC |
| 2501 | AAUCUAGAAAAUGCAUUCA | 5399 | UGAAUGCAUUUUCUAGAUU |
| 2554 | UGUGUCUUUCUCCCCGAGC | 5452 | GCUCGGGGAGAAAGACACA |
| 2639 | GCAGGCACGUGUGUAUGCA | 5537 | UGCAUACACACGUGCCUGC |
| 2646 | CGUGUGUAUGCACUCCCCG | 5544 | CGGGGAGUGCAUACACACG |
| 2647 | GUGUGUAUGCACUCCCCGC | 5545 | GCGGGGAGUGCAUACACAC |
| 2650 | UGUAUGCACUCCCCGCUUG | 5548 | CAAGCGGGGAGUGCAUACA |
| 2653 | AUGCACUCCCCGCUUGUGU | 5551 | ACACAAGCGGGGAGUGCAU |
| 2654 | UGCACUCCCCGCUUGUGUU | 5552 | AACACAAGCGGGGAGUGCA |
| 2660 | CCCCGCUUGUGUUUACACA | 5558 | UGUGUAAACACAAGCGGGG |
| 2750 | UAUAAAUCCCACUCGCUCU | 5648 | AGAGCGAGUGGGAUUUAUA |
| 2752 | UAAAUCCCACUCGCUCUUU | 5650 | AAAGAGCGAGUGGGAUUUA |
| 2753 | AAAUCCCACUCGCUCUUUU | 5651 | AAAAGAGCGAGUGGGAUUU |
| 2754 | AAUCCCACUCGCUCUUUUU | 5652 | AAAAAGAGCGAGUGGGAUU |
| 2785 | AUAACUACUGCAUAAUAUG | 5683 | CAUAUUAUGCAGUAGUUAU |
| 2786 | UAACUACUGCAUAAUAUGG | 5684 | CCAUAUUAUGCAGUAGUUA |
| 2787 | AACUACUGCAUAAUAUGGA | 5685 | UCCAUAUUAUGCAGUAGUU |
| 2788 | ACUACUGCAUAAUAUGGAU | 5686 | AUCCAUAUUAUGCAGUAGU |
| 2793 | UGCAUAAUAUGGAUACGCC | 5691 | GGCGUAUCCAUAUUAUGCA |
| 2794 | GCAUAAUAUGGAUACGCCU | 5692 | AGGCGUAUCCAUAUUAUGC |
| 2796 | AUAAUAUGGAUACGCCUUA | 5694 | UAAGGCGUAUCCAUAUUAU |
| 2797 | UAAUAUGGAUACGCCUUAU | 5695 | AUAAGGCGUAUCCAUAUUA |
| 2798 | AAUAUGGAUACGCCUUAUU | 5696 | AAUAAGGCGUAUCCAUAUU |
| 2799 | AUAUGGAUACGCCUUAUUU | 5697 | AAAUAAGGCGUAUCCAUAU |
| 2800 | UAUGGAUACGCCUUAUUUG | 5698 | CAAAUAAGGCGUAUCCAUA |
| 2802 | UGGAUACGCCUUAUUUGAU | 5700 | AUCAAAUAAGGCGUAUCCA |
| 2803 | GGAUACGCCUUAUUUGAUU | 5701 | AAUCAAAUAAGGCGUAUCC |
| 2804 | GAUACGCCUUAUUUGAUUU | 5702 | AAAUCAAAUAAGGCGUAUC |
| 2806 | UACGCCUUAUUUGAUUUAA | 5704 | UUAAAUCAAAUAAGGCGUA |
| 2807 | ACGCCUUAUUUGAUUUAAC | 5705 | GUUAAAUCAAAUAAGGCGU |
| 2808 | CGCCUUAUUUGAUUUAACU | 5706 | AGUUAAAUCAAAUAAGGCG |
| 2809 | GCCUUAUUUGAUUUAACUA | 5707 | UAGUUAAAUCAAAUAAGGC |
| 2811 | CUUAUUUGAUUUAACUAGU | 5709 | ACUAGUUAAAUCAAAUAAG |
| 2815 | UUUGAUUUAACUAGUUCCC | 5713 | GGGAACUAGUUAAAUCAAA |
| 2817 | UGAUUUAACUAGUUCCCUA | 5715 | UAGGGAACUAGUUAAAUCA |
| 2819 | AUUUAACUAGUUCCCUAAU | 5717 | AUUAGGGAACUAGUUAAAU |
| 2821 | UUAACUAGUUCCCUAAUGA | 5719 | UCAUUAGGGAACUAGUUAA |
| 2824 | ACUAGUUCCCUAAUGAUGG | 5722 | CCAUCAUUAGGGAACUAGU |
| 2825 | CUAGUUCCCUAAUGAUGGA | 5723 | UCCAUCAUUAGGGAACUAG |
| 2826 | UAGUUCCCUAAUGAUGGAC | 5724 | GUCCAUCAUUAGGGAACUA |
| 2827 | AGUUCCCUAAUGAUGGACU | 5725 | AGUCCAUCAUUAGGGAACU |

The siRNAs in subset A have the following characteristics:
Cross-reactivity: With 19mer in human PLIN1 mRNA, with 17mer/19mer in NHP PLIN1
Specificity category: For human and NHP: AS2 or better, SS3 or better
miRNA seeds: AS+SS strand: seed region not conserved in human, mouse, and rat and not present in >4 species
Off-target frequency: ≤20 human off-targets matched with 2 mismatches in antisense strand
SNPs: siRNA target sites do not harbor SNPs with a MAF≥1% (pos, 2-18)

The siRNA sequences in subset A were selected for more stringent specificity to yield subset B. Subset B includes 119 siRNAs whose base sequences are shown in Table 5.

TABLE 5
Sequences in siRNA subset B

| SEQ ID | sense strand sequence | SEQ ID | antisense strand sequence |
|---|---|---|---|
| NO: | (5′-3′) | NO: | (5′-3′) |
| 209 | GCUGCCGGUGGUGAGUGGC | 3107 | GCCACUCACCACCGGCAGC |
| 213 | CCGGUGGUGAGUGGCACCU | 3111 | AGGUGCCACUCACCACCGG |
| 218 | GGUGAGUGGCACCUGCGAA | 3116 | UUCGCAGGUGCCACUCACC |
| 301 | CCUAUGAGAAGGGCGUGCA | 3199 | UGCACGCCCUUCUCAUAGG |
| 302 | CUAUGAGAAGGGCGUGCAG | 3200 | CUGCACGCCCUUCUCAUAG |
| 311 | GGGCGUGCAGAGCGCCAGU | 3209 | ACUGGCGCUCUGCACGCCC |
| 313 | GCGUGCAGAGCGCCAGUAG | 3211 | CUACUGGCGCUCUGCACGC |
| 341 | CUGGAGCAUGGAGCCGGUG | 3239 | CACCGGCUCCAUGCUCCAG |
| 378 | CAGUUCACAGCUGCCAAUG | 3276 | CAUUGGCAGCUGUGAACUG |
| 393 | AAUGAGCUGGCCUGCCGAG | 3291 | CUCGGCAGGCCAGCUCAUU |
| 399 | CUGGCCUGCCGAGGCUUGG | 3297 | CCAAGCCUCGGCAGGCCAG |
| 404 | CUGCCGAGGCUUGGACCAC | 3302 | GUGGUCCAAGCCUCGGCAG |
| 405 | UGCCGAGGCUUGGACCACC | 3303 | GGUGGUCCAAGCCUCGGCA |
| 407 | CCGAGGCUUGGACCACCUG | 3305 | CAGGUGGUCCAAGCCUCGG |
| 501 | CUCCGCAGUGCCAGAAACA | 3399 | UGUUUCUGGCACUGCGGAG |
| 502 | UCCGCAGUGCCAGAAACAG | 3400 | CUGUUUCUGGCACUGCGGA |
| 530 | UCCCAUCGCGAGCACUUCA | 3428 | UGAAGUGCUCGCGAUGGGA |
| 533 | CAUCGCGAGCACUUCAGAC | 3431 | GUCUGAAGUGCUCGCGAUG |
| 534 | AUCGCGAGCACUUCAGACA | 3432 | UGUCUGAAGUGCUCGCGAU |
| 540 | AGCACUUCAGACAAGGUCC | 3438 | GGACCUUGUCUGAAGUGCU |
| 601 | CCAGAGACACUGCGGAAUU | 3499 | AAUUCCGCAGUGUCUCUGG |
| 606 | GACACUGCGGAAUUUGCUG | 3504 | CAGCAAAUUCCGCAGUGUC |
| 610 | CUGCGGAAUUUGCUGCCAA | 3508 | UUGGCAGCAAAUUCCGCAG |
| 616 | AAUUUGCUGCCAACACUCG | 3514 | CGAGUGUUGGCAGCAAAUU |
| 617 | AUUUGCUGCCAACACUCGA | 3515 | UCGAGUGUUGGCAGCAAAU |
| 618 | UUUGCUGCCAACACUCGAG | 3516 | CUCGAGUGUUGGCAGCAAA |
| 619 | UUGCUGCCAACACUCGAGC | 3517 | GCUCGAGUGUUGGCAGCAA |
| 620 | UGCUGCCAACACUCGAGCU | 3518 | AGCUCGAGUGUUGGCAGCA |
| 621 | GCUGCCAACACUCGAGCUG | 3519 | CAGCUCGAGUGUUGGCAGC |
| 622 | CUGCCAACACUCGAGCUGG | 3520 | CCAGCUCGAGUGUUGGCAG |
| 623 | UGCCAACACUCGAGCUGGC | 3521 | GCCAGCUCGAGUGUUGGCA |
| 624 | GCCAACACUCGAGCUGGCC | 3522 | GGCCAGCUCGAGUGUUGGC |
| 625 | CCAACACUCGAGCUGGCCG | 3523 | CGGCCAGCUCGAGUGUUGG |
| 627 | AACACUCGAGCUGGCCGAC | 3525 | GUCGGCCAGCUCGAGUGUU |
| 628 | ACACUCGAGCUGGCCGACU | 3526 | AGUCGGCCAGCUCGAGUGU |
| 629 | CACUCGAGCUGGCCGACUG | 3527 | CAGUCGGCCAGCUCGAGUG |
| 630 | ACUCGAGCUGGCCGACUGG | 3528 | CCAGUCGGCCAGCUCGAGU |
| 631 | CUCGAGCUGGCCGACUGGC | 3529 | GCCAGUCGGCCAGCUCGAG |
| 649 | CUUCUGGAGGGGCCGACUU | 3547 | AAGUCGGCCCCUCCAGAAG |
| 650 | UUCUGGAGGGGCCGACUUG | 3548 | CAAGUCGGCCCCUCCAGAA |
| 690 | GUGGUGGAGUACCUCCUCC | 3588 | GGAGGAGGUACUCCACCAC |
| 692 | GGUGGAGUACCUCCUCCCU | 3590 | AGGGAGGAGGUACUCCACC |
| 797 | UGGGGCUCUGACCAACACC | 3695 | GGUGUUGGUCAGAGCCCCA |
| 943 | CGGUGUCCCGGCGGAGGAG | 3841 | CUCCUCCGCCGGGACACCG |
| 1350 | CUGUCGCUGAUGGAGCCCG | 4248 | CGGGCUCCAUCAGCGACAG |
| 1351 | UGUCGCUGAUGGAGCCCGA | 4249 | UCGGGCUCCAUCAGCGACA |
| 1379 | CCGGGACAUCGACAACCCA | 4277 | UGGGUUGUCGAUGUCCCGG |
| 1400 | AGCCGAGGUCGAGCGCCGG | 4298 | CCGGCGCUCGACCUCGGCU |
| 1401 | GCCGAGGUCGAGCGCCGGG | 4299 | CCCGGCGCUCGACCUCGGC |
| 1404 | GAGGUCGAGCGCCGGGAGG | 4302 | CCUCCCGGCGCUCGACCUC |
| 1551 | GUCGCCACGCCCGCAGCGC | 4449 | GCGCUGCGGGCGUGGCGAC |
| 1607 | AAAGCGCAGGGUCAGCGAC | 4505 | GUCGCUGACCCUGCGCUUU |
| 1609 | AGCGCAGGGUCAGCGACAG | 4507 | CUGUCGCUGACCCUGCGCU |
| 1612 | GCAGGGUCAGCGACAGCUU | 4510 | AAGCUGUCGCUGACCCUGC |
| 1613 | CAGGGUCAGCGACAGCUUC | 4511 | GAAGCUGUCGCUGACCCUG |
| 1614 | AGGGUCAGCGACAGCUUCU | 4512 | AGAAGCUGUCGCUGACCCU |
| 1619 | CAGCGACAGCUUCUUCCGG | 4517 | CCGGAAGAAGCUGUCGCUG |
| 1620 | AGCGACAGCUUCUUCCGGC | 4518 | GCCGGAAGAAGCUGUCGCU |
| 1630 | UCUUCCGGCCCAGCGUCAU | 4528 | AUGACGCUGGGCCGGAAGA |
| 1631 | CUUCCGGCCCAGCGUCAUG | 4529 | CAUGACGCUGGGCCGGAAG |
| 1632 | UUCCGGCCCAGCGUCAUGG | 4530 | CCAUGACGCUGGGCCGGAA |
| 1687 | GCAAGAAGAGCUGAGUCGC | 4585 | GCGACUCAGCUCUUCUUGC |
| 1692 | AAGAGCUGAGUCGCCGCAC | 4590 | GUGCGGCGACUCAGCUCUU |
| 1905 | AGCCAGUUUUUAAGGGACA | 4803 | UGUCCCUUAAAAACUGGCU |
| 1907 | CCAGUUUUUAAGGGACACC | 4805 | GGUGUCCCUUAAAAACUGG |
| 1908 | CAGUUUUUAAGGGACACCA | 4806 | UGGUGUCCCUUAAAAACUG |
| 1909 | AGUUUUUAAGGGACACCAG | 4807 | CUGGUGUCCCUUAAAAACU |
| 1911 | UUUUUAAGGGACACCAGAG | 4809 | CUCUGGUGUCCCUUAAAAA |
| 1930 | CCGCAGCCUGCUCUGAUUC | 4828 | GAAUCAGAGCAGGCUGCGG |
| 1934 | AGCCUGCUCUGAUUCUAUG | 4832 | CAUAGAAUCAGAGCAGGCU |
| 1968 | UAAGAGUAAUUGCCUAACU | 4866 | AGUUAGGCAAUUACUCUUA |
| 1969 | AAGAGUAAUUGCCUAACUU | 4867 | AAGUUAGGCAAUUACUCUU |
| 1973 | GUAAUUGCCUAACUUGAUU | 4871 | AAUCAAGUUAGGCAAUUAC |
| 1974 | UAAUUGCCUAACUUGAUUU | 4872 | AAAUCAAGUUAGGCAAUUA |
| 2002 | UUAACCAAACUUGUGGCCA | 4900 | UGGCCACAAGUUUGGUUAA |
| 2076 | CCACGAAUGAGUAACUCCU | 4974 | AGGAGUUACUCAUUCGUGG |
| 2080 | GAAUGAGUAACUCCUGUCA | 4978 | UGACAGGAGUUACUCAUUC |
| 2124 | UUUUGACACAUUCUUAGCA | 5022 | UGCUAAGAAUGUGUCAAAA |
| 2125 | UUUGACACAUUCUUAGCAC | 5023 | GUGCUAAGAAUGUGUCAAA |
| 2126 | UUGACACAUUCUUAGCACU | 5024 | AGUGCUAAGAAUGUGUCAA |
| 2275 | AGUGUCACUUUCUGAGUAC | 5173 | GUACUCAGAAAGUGACACU |
| 2276 | GUGUCACUUUCUGAGUACC | 5174 | GGUACUCAGAAAGUGACAC |
| 2386 | CACCUGCAUAGUCACUCUU | 5284 | AAGAGUGACUAUGCAGGUG |
| 2390 | UGCAUAGUCACUCUUUUGA | 5288 | UCAAAAGAGUGACUAUGCA |
| 2394 | UAGUCACUCUUUUGAUGCU | 5292 | AGCAUCAAAAGAGUGACUA |
| 2448 | GUAUAGCCACAGUUCAUCC | 5346 | GGAUGAACUGUGGCUAUAC |
| 2554 | UGUGUCUUUCUCCCCGAGC | 5452 | GCUCGGGGAGAAAGACACA |
| 2646 | CGUGUGUAUGCACUCCCCG | 5544 | CGGGGAGUGCAUACACACG |
| 2647 | GUGUGUAUGCACUCCCCGC | 5545 | GCGGGGAGUGCAUACACAC |
| 2650 | UGUAUGCACUCCCCGCUUG | 5548 | CAAGCGGGGAGUGCAUACA |
| 2653 | AUGCACUCCCCGCUUGUGU | 5551 | ACACAAGCGGGGAGUGCAU |
| 2654 | UGCACUCCCCGCUUGUGUU | 5552 | AACACAAGCGGGGAGUGCA |
| 2660 | CCCCGCUUGUGUUUACACA | 5558 | UGUGUAAACACAAGCGGGG |
| 2750 | UAUAAAUCCCACUCGCUCU | 5648 | AGAGCGAGUGGGAUUUAUA |
| 2752 | UAAAUCCCACUCGCUCUUU | 5650 | AAAGAGCGAGUGGGAUUUA |
| 2753 | AAAUCCCACUCGCUCUUUU | 5651 | AAAAGAGCGAGUGGGAUUU |
| 2754 | AAUCCCACUCGCUCUUUUU | 5652 | AAAAAGAGCGAGUGGGAUU |
| 2787 | AACUACUGCAUAAUAUGGA | 5685 | UCCAUAUUAUGCAGUAGUU |
| 2788 | ACUACUGCAUAAUAUGGAU | 5686 | AUCCAUAUUAUGCAGUAGU |
| 2793 | UGCAUAAUAUGGAUACGCC | 5691 | GGCGUAUCCAUAUUAUGCA |
| 2794 | GCAUAAUAUGGAUACGCCU | 5692 | AGGCGUAUCCAUAUUAUGC |
| 2796 | AUAAUAUGGAUACGCCUUA | 5694 | UAAGGCGUAUCCAUAUUAU |
| 2797 | UAAUAUGGAUACGCCUUAU | 5695 | AUAAGGCGUAUCCAUAUUA |
| 2798 | AAUAUGGAUACGCCUUAUU | 5696 | AAUAAGGCGUAUCCAUAUU |
| 2799 | AUAUGGAUACGCCUUAUUU | 5697 | AAAUAAGGCGUAUCCAUAU |
| 2800 | UAUGGAUACGCCUUAUUUG | 5698 | CAAAUAAGGCGUAUCCAUA |
| 2802 | UGGAUACGCCUUAUUUGAU | 5700 | AUCAAAUAAGGCGUAUCCA |
| 2803 | GGAUACGCCUUAUUUGAUU | 5701 | AAUCAAAUAAGGCGUAUCC |
| 2804 | GAUACGCCUUAUUUGAUUU | 5702 | AAAUCAAAUAAGGCGUAUC |
| 2806 | UACGCCUUAUUUGAUUUAA | 5704 | UUAAAUCAAAUAAGGCGUA |
| 2807 | ACGCCUUAUUUGAUUUAAC | 5705 | GUUAAAUCAAAUAAGGCGU |
| 2811 | CUUAUUUGAUUUAACUAGU | 5709 | ACUAGUUAAAUCAAAUAAG |
| 2817 | UGAUUUAACUAGUUCCCUA | 5715 | UAGGGAACUAGUUAAAUCA |
| 2819 | AUUUAACUAGUUCCCUAAU | 5717 | AUUAGGGAACUAGUUAAAU |
| 2821 | UUAACUAGUUCCCUAAUGA | 5719 | UCAUUAGGGAACUAGUUAA |
| 2824 | ACUAGUUCCCUAAUGAUGG | 5722 | CCAUCAUUAGGGAACUAGU |
| 2825 | CUAGUUCCCUAAUGAUGGA | 5723 | UCCAUCAUUAGGGAACUAG |
| 2826 | UAGUUCCCUAAUGAUGGAC | 5724 | GUCCAUCAUUAGGGAACUA |
| 2827 | AGUUCCCUAAUGAUGGACU | 5725 | AGUCCAUCAUUAGGGAACU |

The siRNAs in subset B have the following characteristics:
Cross-reactivity: With 19mer in human PLIN1 mRNA, with 17mer/19mer in NHP PLIN1
Specificity category: For human and NHP: AS2 or better, SS3 or better
miRNA seeds: AS+SS strand: seed region not conserved in human, mouse, and rat and not present in >4 species
Off-target frequency: ≤15 human off-targets matched with 2 mismatches in antisense strand
SNPs: siRNA target sites do not harbor SNPs with a MAF≥1% (pos, 2-18)

The siRNA sequences in subset B were further selected for absence of seed regions in the AS strand that are identical to a seed region of known human miRNA to yield subset C. Subset C includes 77 siRNAs whose base sequences are shown in Table 6.

TABLE 6
Sequences in siRNA subset C

| SEQ ID | sense strand sequence | SEQ ID | antisense strand sequence |
|---|---|---|---|
| NO: | (5′-3′) | NO: | (5′-3′) |
| 218 | GGUGAGUGGCACCUGCGAA | 3116 | UUCGCAGGUGCCACUCACC |
| 301 | CCUAUGAGAAGGGCGUGCA | 3199 | UGCACGCCCUUCUCAUAGG |
| 302 | CUAUGAGAAGGGCGUGCAG | 3200 | CUGCACGCCCUUCUCAUAG |
| 311 | GGGCGUGCAGAGCGCCAGU | 3209 | ACUGGCGCUCUGCACGCCC |
| 313 | GCGUGCAGAGCGCCAGUAG | 3211 | CUACUGGCGCUCUGCACGC |
| 341 | CUGGAGCAUGGAGCCGGUG | 3239 | CACCGGCUCCAUGCUCCAG |
| 378 | CAGUUCACAGCUGCCAAUG | 3276 | CAUUGGCAGCUGUGAACUG |
| 393 | AAUGAGCUGGCCUGCCGAG | 3291 | CUCGGCAGGCCAGCUCAUU |
| 399 | CUGGCCUGCCGAGGCUUGG | 3297 | CCAAGCCUCGGCAGGCCAG |
| 404 | CUGCCGAGGCUUGGACCAC | 3302 | GUGGUCCAAGCCUCGGCAG |
| 405 | UGCCGAGGCUUGGACCACC | 3303 | GGUGGUCCAAGCCUCGGCA |
| 501 | CUCCGCAGUGCCAGAAACA | 3399 | UGUUUCUGGCACUGCGGAG |
| 502 | UCCGCAGUGCCAGAAACAG | 3400 | CUGUUUCUGGCACUGCGGA |
| 533 | CAUCGCGAGCACUUCAGAC | 3431 | GUCUGAAGUGCUCGCGAUG |
| 534 | AUCGCGAGCACUUCAGACA | 3432 | UGUCUGAAGUGCUCGCGAU |
| 601 | CCAGAGACACUGCGGAAUU | 3499 | AAUUCCGCAGUGUCUCUGG |
| 617 | AUUUGCUGCCAACACUCGA | 3515 | UCGAGUGUUGGCAGCAAAU |
| 619 | UUGCUGCCAACACUCGAGC | 3517 | GCUCGAGUGUUGGCAGCAA |
| 620 | UGCUGCCAACACUCGAGCU | 3518 | AGCUCGAGUGUUGGCAGCA |
| 621 | GCUGCCAACACUCGAGCUG | 3519 | CAGCUCGAGUGUUGGCAGC |
| 625 | CCAACACUCGAGCUGGCCG | 3523 | CGGCCAGCUCGAGUGUUGG |
| 628 | ACACUCGAGCUGGCCGACU | 3526 | AGUCGGCCAGCUCGAGUGU |
| 629 | CACUCGAGCUGGCCGACUG | 3527 | CAGUCGGCCAGCUCGAGUG |
| 630 | ACUCGAGCUGGCCGACUGG | 3528 | CCAGUCGGCCAGCUCGAGU |
| 631 | CUCGAGCUGGCCGACUGGC | 3529 | GCCAGUCGGCCAGCUCGAG |
| 649 | CUUCUGGAGGGGCCGACUU | 3547 | AAGUCGGCCCCUCCAGAAG |
| 650 | UUCUGGAGGGGCCGACUUG | 3548 | CAAGUCGGCCCCUCCAGAA |
| 797 | UGGGGCUCUGACCAACACC | 3695 | GGUGUUGGUCAGAGCCCCA |
| 1351 | UGUCGCUGAUGGAGCCCGA | 4249 | UCGGGCUCCAUCAGCGACA |
| 1400 | AGCCGAGGUCGAGCGCCGG | 4298 | CCGGCGCUCGACCUCGGCU |
| 1401 | GCCGAGGUCGAGCGCCGGG | 4299 | CCCGGCGCUCGACCUCGGC |
| 1404 | GAGGUCGAGCGCCGGGAGG | 4302 | CCUCCCGGCGCUCGACCUC |
| 1551 | GUCGCCACGCCCGCAGCGC | 4449 | GCGCUGCGGGCGUGGCGAC |
| 1609 | AGCGCAGGGUCAGCGACAG | 4507 | CUGUCGCUGACCCUGCGCU |
| 1612 | GCAGGGUCAGCGACAGCUU | 4510 | AAGCUGUCGCUGACCCUGC |
| 1620 | AGCGACAGCUUCUUCCGGC | 4518 | GCCGGAAGAAGCUGUCGCU |
| 1630 | UCUUCCGGCCCAGCGUCAU | 4528 | AUGACGCUGGGCCGGAAGA |
| 1632 | UUCCGGCCCAGCGUCAUGG | 4530 | CCAUGACGCUGGGCCGGAA |
| 1692 | AAGAGCUGAGUCGCCGCAC | 4590 | GUGCGGCGACUCAGCUCUU |
| 1905 | AGCCAGUUUUUAAGGGACA | 4803 | UGUCCCUUAAAAACUGGCU |
| 1908 | CAGUUUUUAAGGGACACCA | 4806 | UGGUGUCCCUUAAAAACUG |
| 1909 | AGUUUUUAAGGGACACCAG | 4807 | CUGGUGUCCCUUAAAAACU |
| 1911 | UUUUUAAGGGACACCAGAG | 4809 | CUCUGGUGUCCCUUAAAAA |
| 1934 | AGCCUGCUCUGAUUCUAUG | 4832 | CAUAGAAUCAGAGCAGGCU |
| 1969 | AAGAGUAAUUGCCUAACUU | 4867 | AAGUUAGGCAAUUACUCUU |
| 1973 | GUAAUUGCCUAACUUGAUU | 4871 | AAUCAAGUUAGGCAAUUAC |
| 1974 | UAAUUGCCUAACUUGAUUU | 4872 | AAAUCAAGUUAGGCAAUUA |
| 2002 | UUAACCAAACUUGUGGCCA | 4900 | UGGCCACAAGUUUGGUUAA |
| 2080 | GAAUGAGUAACUCCUGUCA | 4978 | UGACAGGAGUUACUCAUUC |
| 2124 | UUUUGACACAUUCUUAGCA | 5022 | UGCUAAGAAUGUGUCAAAA |
| 2126 | UUGACACAUUCUUAGCACU | 5024 | AGUGCUAAGAAUGUGUCAA |
| 2275 | AGUGUCACUUUCUGAGUAC | 5173 | GUACUCAGAAAGUGACACU |
| 2276 | GUGUCACUUUCUGAGUACC | 5174 | GGUACUCAGAAAGUGACAC |
| 2390 | UGCAUAGUCACUCUUUUGA | 5288 | UCAAAAGAGUGACUAUGCA |
| 2448 | GUAUAGCCACAGUUCAUCC | 5346 | GGAUGAACUGUGGCUAUAC |
| 2554 | UGUGUCUUUCUCCCCGAGC | 5452 | GCUCGGGGAGAAAGACACA |
| 2650 | UGUAUGCACUCCCCGCUUG | 5548 | CAAGCGGGGAGUGCAUACA |
| 2660 | CCCCGCUUGUGUUUACACA | 5558 | UGUGUAAACACAAGCGGGG |
| 2750 | UAUAAAUCCCACUCGCUCU | 5648 | AGAGCGAGUGGGAUUUAUA |
| 2752 | UAAAUCCCACUCGCUCUUU | 5650 | AAAGAGCGAGUGGGAUUUA |
| 2754 | AAUCCCACUCGCUCUUUUU | 5652 | AAAAAGAGCGAGUGGGAUU |
| 2787 | AACUACUGCAUAAUAUGGA | 5685 | UCCAUAUUAUGCAGUAGUU |
| 2788 | ACUACUGCAUAAUAUGGAU | 5686 | AUCCAUAUUAUGCAGUAGU |
| 2793 | UGCAUAAUAUGGAUACGCC | 5691 | GGCGUAUCCAUAUUAUGCA |
| 2794 | GCAUAAUAUGGAUACGCCU | 5692 | AGGCGUAUCCAUAUUAUGC |
| 2797 | UAAUAUGGAUACGCCUUAU | 5695 | AUAAGGCGUAUCCAUAUUA |
| 2798 | AAUAUGGAUACGCCUUAUU | 5696 | AAUAAGGCGUAUCCAUAUU |
| 2799 | AUAUGGAUACGCCUUAUUU | 5697 | AAAUAAGGCGUAUCCAUAU |
| 2803 | GGAUACGCCUUAUUUGAUU | 5701 | AAUCAAAUAAGGCGUAUCC |
| 2804 | GAUACGCCUUAUUUGAUUU | 5702 | AAAUCAAAUAAGGCGUAUC |
| 2806 | UACGCCUUAUUUGAUUUAA | 5704 | UUAAAUCAAAUAAGGCGUA |
| 2807 | ACGCCUUAUUUGAUUUAAC | 5705 | GUUAAAUCAAAUAAGGCGU |
| 2811 | CUUAUUUGAUUUAACUAGU | 5709 | ACUAGUUAAAUCAAAUAAG |
| 2821 | UUAACUAGUUCCCUAAUGA | 5719 | UCAUUAGGGAACUAGUUAA |
| 2824 | ACUAGUUCCCUAAUGAUGG | 5722 | CCAUCAUUAGGGAACUAGU |
| 2826 | UAGUUCCCUAAUGAUGGAC | 5724 | GUCCAUCAUUAGGGAACUA |
| 2827 | AGUUCCCUAAUGAUGGACU | 5725 | AGUCCAUCAUUAGGGAACU |

The siRNAs in subset C have the following characteristics:
Cross-reactivity: With 19mer in human PLIN1 mRNA, with 17mer/19mer in NHP PLIN1
Specificity category: For human and NHP: AS2 or better, SS3 or better
miRNA seeds: AS+SS strand: seed region not conserved in human, mouse, and rat and not present in >4 species. AS strand: seed region not identical to seed region of known human miRNA
Off-target frequency: ≤15 human off-targets matched with 2 mismatches by antisense strand
SNPs: siRNA target sites do not harbor SNPs with a MAF≥1% (pos, 2-18)

The siRNA sequences in subset C were also selected for absence of seed regions in the AS or S strands that are identical to a seed region of known human miRNA to yield subset D. Subset D includes 62 siRNAs whose base sequences are shown in Table 7.

TABLE 7
Sequences in siRNA subset D

| SEQ ID | sense strand sequence | SEQ ID | antisense strand sequence |
|---|---|---|---|
| NO: | (5′-3′) | NO: | (5′-3′) |
| 218 | GGUGAGUGGCACCUGCGAA | 3116 | UUCGCAGGUGCCACUCACC |
| 300 | GCCUAUGAGAAGGGCGUGC | 3198 | GCACGCCCUUCUCAUAGGC |
| 301 | CCUAUGAGAAGGGCGUGCA | 3199 | UGCACGCCCUUCUCAUAGG |
| 302 | CUAUGAGAAGGGCGUGCAG | 3200 | CUGCACGCCCUUCUCAUAG |
| 311 | GGGCGUGCAGAGCGCCAGU | 3209 | ACUGGCGCUCUGCACGCCC |
| 344 | GAGCAUGGAGCCGGUGGUC | 3242 | GACCACCGGCUCCAUGCUC |
| 404 | CUGCCGAGGCUUGGACCAC | 3302 | GUGGUCCAAGCCUCGGCAG |
| 405 | UGCCGAGGCUUGGACCACC | 3303 | GGUGGUCCAAGCCUCGGCA |
| 501 | CUCCGCAGUGCCAGAAACA | 3399 | UGUUUCUGGCACUGCGGAG |
| 503 | CCGCAGUGCCAGAAACAGC | 3401 | GCUGUUUCUGGCACUGCGG |
| 533 | CAUCGCGAGCACUUCAGAC | 3431 | GUCUGAAGUGCUCGCGAUG |
| 534 | AUCGCGAGCACUUCAGACA | 3432 | UGUCUGAAGUGCUCGCGAU |
| 619 | UUGCUGCCAACACUCGAGC | 3517 | GCUCGAGUGUUGGCAGCAA |
| 625 | CCAACACUCGAGCUGGCCG | 3523 | CGGCCAGCUCGAGUGUUGG |
| 628 | ACACUCGAGCUGGCCGACU | 3526 | AGUCGGCCAGCUCGAGUGU |
| 629 | CACUCGAGCUGGCCGACUG | 3527 | CAGUCGGCCAGCUCGAGUG |
| 630 | ACUCGAGCUGGCCGACUGG | 3528 | CCAGUCGGCCAGCUCGAGU |
| 631 | CUCGAGCUGGCCGACUGGC | 3529 | GCCAGUCGGCCAGCUCGAG |
| 634 | GAGCUGGCCGACUGGCUUC | 3532 | GAAGCCAGUCGGCCAGCUC |
| 649 | CUUCUGGAGGGGCCGACUU | 3547 | AAGUCGGCCCCUCCAGAAG |
| 935 | CAUGCAGGCGGUGUCCCGG | 3833 | CCGGGACACCGCCUGCAUG |
| 1351 | UGUCGCUGAUGGAGCCCGA | 4249 | UCGGGCUCCAUCAGCGACA |
| 1400 | AGCCGAGGUCGAGCGCCGG | 4298 | CCGGCGCUCGACCUCGGCU |
| 1401 | GCCGAGGUCGAGCGCCGGG | 4299 | CCCGGCGCUCGACCUCGGC |
| 1608 | AAGCGCAGGGUCAGCGACA | 4506 | UGUCGCUGACCCUGCGCUU |
| 1609 | AGCGCAGGGUCAGCGACAG | 4507 | CUGUCGCUGACCCUGCGCU |
| 1630 | UCUUCCGGCCCAGCGUCAU | 4528 | AUGACGCUGGGCCGGAAGA |
| 1632 | UUCCGGCCCAGCGUCAUGG | 4530 | CCAUGACGCUGGGCCGGAA |
| 1692 | AAGAGCUGAGUCGCCGCAC | 4590 | GUGCGGCGACUCAGCUCUU |
| 1905 | AGCCAGUUUUUAAGGGACA | 4803 | UGUCCCUUAAAAACUGGCU |
| 1908 | CAGUUUUUAAGGGACACCA | 4806 | UGGUGUCCCUUAAAAACUG |
| 1909 | AGUUUUUAAGGGACACCAG | 4807 | CUGGUGUCCCUUAAAAACU |
| 1911 | UUUUUAAGGGACACCAGAG | 4809 | CUCUGGUGUCCCUUAAAAA |
| 1969 | AAGAGUAAUUGCCUAACUU | 4867 | AAGUUAGGCAAUUACUCUU |
| 1973 | GUAAUUGCCUAACUUGAUU | 4871 | AAUCAAGUUAGGCAAUUAC |
| 2002 | UUAACCAAACUUGUGGCCA | 4900 | UGGCCACAAGUUUGGUUAA |
| 2080 | GAAUGAGUAACUCCUGUCA | 4978 | UGACAGGAGUUACUCAUUC |
| 2124 | UUUUGACACAUUCUUAGCA | 5022 | UGCUAAGAAUGUGUCAAAA |
| 2126 | UUGACACAUUCUUAGCACU | 5024 | AGUGCUAAGAAUGUGUCAA |
| 2131 | ACAUUCUUAGCACUGAACU | 5029 | AGUUCAGUGCUAAGAAUGU |
| 2276 | GUGUCACUUUCUGAGUACC | 5174 | GGUACUCAGAAAGUGACAC |
| 2390 | UGCAUAGUCACUCUUUUGA | 5288 | UCAAAAGAGUGACUAUGCA |
| 2448 | GUAUAGCCACAGUUCAUCC | 5346 | GGAUGAACUGUGGCUAUAC |
| 2501 | AAUCUAGAAAAUGCAUUCA | 5399 | UGAAUGCAUUUUCUAGAUU |
| 2650 | UGUAUGCACUCCCCGCUUG | 5548 | CAAGCGGGGAGUGCAUACA |
| 2660 | CCCCGCUUGUGUUUACACA | 5558 | UGUGUAAACACAAGCGGGG |
| 2750 | UAUAAAUCCCACUCGCUCU | 5648 | AGAGCGAGUGGGAUUUAUA |
| 2787 | AACUACUGCAUAAUAUGGA | 5685 | UCCAUAUUAUGCAGUAGUU |
| 2788 | ACUACUGCAUAAUAUGGAU | 5686 | AUCCAUAUUAUGCAGUAGU |
| 2793 | UGCAUAAUAUGGAUACGCC | 5691 | GGCGUAUCCAUAUUAUGCA |
| 2794 | GCAUAAUAUGGAUACGCCU | 5692 | AGGCGUAUCCAUAUUAUGC |
| 2799 | AUAUGGAUACGCCUUAUUU | 5697 | AAAUAAGGCGUAUCCAUAU |
| 2803 | GGAUACGCCUUAUUUGAUU | 5701 | AAUCAAAUAAGGCGUAUCC |
| 2804 | GAUACGCCUUAUUUGAUUU | 5702 | AAAUCAAAUAAGGCGUAUC |
| 2806 | UACGCCUUAUUUGAUUUAA | 5704 | UUAAAUCAAAUAAGGCGUA |
| 2808 | CGCCUUAUUUGAUUUAACU | 5706 | AGUUAAAUCAAAUAAGGCG |
| 2809 | GCCUUAUUUGAUUUAACUA | 5707 | UAGUUAAAUCAAAUAAGGC |
| 2811 | CUUAUUUGAUUUAACUAGU | 5709 | ACUAGUUAAAUCAAAUAAG |
| 2815 | UUUGAUUUAACUAGUUCCC | 5713 | GGGAACUAGUUAAAUCAAA |
| 2821 | UUAACUAGUUCCCUAAUGA | 5719 | UCAUUAGGGAACUAGUUAA |
| 2824 | ACUAGUUCCCUAAUGAUGG | 5722 | CCAUCAUUAGGGAACUAGU |
| 2826 | UAGUUCCCUAAUGAUGGAC | 5724 | GUCCAUCAUUAGGGAACUA |

The siRNAs in subset D have the following characteristics:
Cross-reactivity: With 19mer in human PLIN1 mRNA, with 17mer/19mer in NHP PLIN1
Specificity category: For human and NHP: AS2 or better, SS3 or better
miRNA seeds: AS+SS strand: seed region not conserved in human, mouse, and rat and not present in >4 species. AS+SS strand: seed region not identical to seed region of known human miRNA
Off-target frequency: ≤20 human off-targets matched with 2 mismatches by antisense strand
SNPs: siRNA target sites do not harbor SNPs with a MAF≥1% (pos, 2-18)

The siRNA sequences in subset D were further selected for more stringent specificity to yield subset E. Subset E includes 51 siRNAs whose base sequences are shown in Table 8.

TABLE 8
Sequences in siRNA subset E

| SEQ ID | sense strand sequence | SEQ ID | antisense strand sequence |
|---|---|---|---|
| NO: | (5′-3′) | NO: | (5′-3′) |
| 218 | GGUGAGUGGCACCUGCGAA | 3116 | UUCGCAGGUGCCACUCACC |
| 301 | CCUAUGAGAAGGGCGUGCA | 3199 | UGCACGCCCUUCUCAUAGG |
| 302 | CUAUGAGAAGGGCGUGCAG | 3200 | CUGCACGCCCUUCUCAUAG |
| 311 | GGGCGUGCAGAGCGCCAGU | 3209 | ACUGGCGCUCUGCACGCCC |
| 404 | CUGCCGAGGCUUGGACCAC | 3302 | GUGGUCCAAGCCUCGGCAG |
| 405 | UGCCGAGGCUUGGACCACC | 3303 | GGUGGUCCAAGCCUCGGCA |
| 501 | CUCCGCAGUGCCAGAAACA | 3399 | UGUUUCUGGCACUGCGGAG |
| 533 | CAUCGCGAGCACUUCAGAC | 3431 | GUCUGAAGUGCUCGCGAUG |
| 534 | AUCGCGAGCACUUCAGACA | 3432 | UGUCUGAAGUGCUCGCGAU |
| 619 | UUGCUGCCAACACUCGAGC | 3517 | GCUCGAGUGUUGGCAGCAA |
| 625 | CCAACACUCGAGCUGGCCG | 3523 | CGGCCAGCUCGAGUGUUGG |
| 628 | ACACUCGAGCUGGCCGACU | 3526 | AGUCGGCCAGCUCGAGUGU |
| 629 | CACUCGAGCUGGCCGACUG | 3527 | CAGUCGGCCAGCUCGAGUG |
| 630 | ACUCGAGCUGGCCGACUGG | 3528 | CCAGUCGGCCAGCUCGAGU |
| 631 | CUCGAGCUGGCCGACUGGC | 3529 | GCCAGUCGGCCAGCUCGAG |
| 649 | CUUCUGGAGGGGCCGACUU | 3547 | AAGUCGGCCCCUCCAGAAG |
| 1351 | UGUCGCUGAUGGAGCCCGA | 4249 | UCGGGCUCCAUCAGCGACA |
| 1400 | AGCCGAGGUCGAGCGCCGG | 4298 | CCGGCGCUCGACCUCGGCU |
| 1401 | GCCGAGGUCGAGCGCCGGG | 4299 | CCCGGCGCUCGACCUCGGC |
| 1609 | AGCGCAGGGUCAGCGACAG | 4507 | CUGUCGCUGACCCUGCGCU |
| 1630 | UCUUCCGGCCCAGCGUCAU | 4528 | AUGACGCUGGGCCGGAAGA |
| 1632 | UUCCGGCCCAGCGUCAUGG | 4530 | CCAUGACGCUGGGCCGGAA |
| 1692 | AAGAGCUGAGUCGCCGCAC | 4590 | GUGCGGCGACUCAGCUCUU |
| 1905 | AGCCAGUUUUUAAGGGACA | 4803 | UGUCCCUUAAAAACUGGCU |
| 1908 | CAGUUUUUAAGGGACACCA | 4806 | UGGUGUCCCUUAAAAACUG |
| 1909 | AGUUUUUAAGGGACACCAG | 4807 | CUGGUGUCCCUUAAAAACU |
| 1911 | UUUUUAAGGGACACCAGAG | 4809 | CUCUGGUGUCCCUUAAAAA |
| 1969 | AAGAGUAAUUGCCUAACUU | 4867 | AAGUUAGGCAAUUACUCUU |
| 1973 | GUAAUUGCCUAACUUGAUU | 4871 | AAUCAAGUUAGGCAAUUAC |
| 2002 | UUAACCAAACUUGUGGCCA | 4900 | UGGCCACAAGUUUGGUUAA |
| 2080 | GAAUGAGUAACUCCUGUCA | 4978 | UGACAGGAGUUACUCAUUC |
| 2124 | UUUUGACACAUUCUUAGCA | 5022 | UGCUAAGAAUGUGUCAAAA |
| 2126 | UUGACACAUUCUUAGCACU | 5024 | AGUGCUAAGAAUGUGUCAA |
| 2276 | GUGUCACUUUCUGAGUACC | 5174 | GGUACUCAGAAAGUGACAC |
| 2390 | UGCAUAGUCACUCUUUUGA | 5288 | UCAAAAGAGUGACUAUGCA |
| 2448 | GUAUAGCCACAGUUCAUCC | 5346 | GGAUGAACUGUGGCUAUAC |
| 2650 | UGUAUGCACUCCCCGCUUG | 5548 | CAAGCGGGGAGUGCAUACA |
| 2660 | CCCCGCUUGUGUUUACACA | 5558 | UGUGUAAACACAAGCGGGG |
| 2750 | UAUAAAUCCCACUCGCUCU | 5648 | AGAGCGAGUGGGAUUUAUA |
| 2787 | AACUACUGCAUAAUAUGGA | 5685 | UCCAUAUUAUGCAGUAGUU |
| 2788 | ACUACUGCAUAAUAUGGAU | 5686 | AUCCAUAUUAUGCAGUAGU |
| 2793 | UGCAUAAUAUGGAUACGCC | 5691 | GGCGUAUCCAUAUUAUGCA |
| 2794 | GCAUAAUAUGGAUACGCCU | 5692 | AGGCGUAUCCAUAUUAUGC |
| 2799 | AUAUGGAUACGCCUUAUUU | 5697 | AAAUAAGGCGUAUCCAUAU |
| 2803 | GGAUACGCCUUAUUUGAUU | 5701 | AAUCAAAUAAGGCGUAUCC |
| 2804 | GAUACGCCUUAUUUGAUUU | 5702 | AAAUCAAAUAAGGCGUAUC |
| 2806 | UACGCCUUAUUUGAUUUAA | 5704 | UUAAAUCAAAUAAGGCGUA |
| 2811 | CUUAUUUGAUUUAACUAGU | 5709 | ACUAGUUAAAUCAAAUAAG |
| 2821 | UUAACUAGUUCCCUAAUGA | 5719 | UCAUUAGGGAACUAGUUAA |
| 2824 | ACUAGUUCCCUAAUGAUGG | 5722 | CCAUCAUUAGGGAACUAGU |
| 2826 | UAGUUCCCUAAUGAUGGAC | 5724 | GUCCAUCAUUAGGGAACUA |

The siRNAs in subset E have the following characteristics:
Cross-reactivity: With 19mer in human PLIN1 mRNA, with 17mer/19mer in NHP PLIN1
Specificity category: For human and NHP: AS2 or better, SS3 or better
miRNA seeds: AS+SS strand: seed region not conserved in human, mouse, and rat and not present in >4 species. AS+SS strand: seed region not identical to seed region of known human miRNA
Off-target frequency: ≤15 human off-targets matched with 2 mismatches by antisense strand
SNPs: siRNA target sites do not harbor SNPs with a MAF≥1% (pos, 2-18)

Subset F includes 49 siRNAs. The siRNAs in subset F include siRNAs from subset A, and are included in Table 9. In some cases, the sense strand of any of the siRNAs of subset F comprises modification pattern 6S (Table 10). In some cases, the antisense strand of any of the siRNAs of subset F comprises modification pattern 7AS (Table 10). In some cases, the sense strand of any of the siRNAs of subset F contains an alternative modification pattern (Table 11). In some cases, the antisense strand of any of the siRNAs of subset F comprises modification pattern 7AS (Table 11). The siRNAs in subset F may comprise any other modification pattern(s). In Table 10 and Table 11, Nf (e.g. Af, Cf, Gf, Tf, or Uf) is a 2″ fluoro-modified nucleoside, n (e.g. a, c, g, t, or u) is a 2′ O-methyl modified nucleoside, and “s” is a phosphorothioate linkage.

TABLE 9
Sequences in siRNA subset F

| SEQ ID | sense strand sequence | SEQ ID | antisense strand sequence |
|---|---|---|---|
| NO: | (5′-3′) | NO: | (5′-3′) |
| 378 | CAGUUCACAGCUGCCAAUG | 3276 | CAUUGGCAGCUGUGAACUG |
| 540 | AGCACUUCAGACAAGGUCC | 3438 | GGACCUUGUCUGAAGUGCU |
| 601 | CCAGAGACACUGCGGAAUU | 3499 | AAUUCCGCAGUGUCUCUGG |
| 610 | CUGCGGAAUUUGCUGCCAA | 3508 | UUGGCAGCAAAUUCCGCAG |
| 1905 | AGCCAGUUUUUAAGGGACA | 4803 | UGUCCCUUAAAAACUGGCU |
| 1907 | CCAGUUUUUAAGGGACACC | 4805 | GGUGUCCCUUAAAAACUGG |
| 1908 | CAGUUUUUAAGGGACACCA | 4806 | UGGUGUCCCUUAAAAACUG |
| 1911 | UUUUUAAGGGACACCAGAG | 4809 | CUCUGGUGUCCCUUAAAAA |
| 1913 | UUUAAGGGACACCAGAGCC | 4811 | GGCUCUGGUGUCCCUUAAA |
| 1934 | AGCCUGCUCUGAUUCUAUG | 4832 | CAUAGAAUCAGAGCAGGCU |
| 1968 | UAAGAGUAAUUGCCUAACU | 4866 | AGUUAGGCAAUUACUCUUA |
| 1973 | GUAAUUGCCUAACUUGAUU | 4871 | AAUCAAGUUAGGCAAUUAC |
| 1974 | UAAUUGCCUAACUUGAUUU | 4872 | AAAUCAAGUUAGGCAAUUA |
| 2002 | UUAACCAAACUUGUGGCCA | 4900 | UGGCCACAAGUUUGGUUAA |
| 2080 | GAAUGAGUAACUCCUGUCA | 4978 | UGACAGGAGUUACUCAUUC |
| 2124 | UUUUGACACAUUCUUAGCA | 5022 | UGCUAAGAAUGUGUCAAAA |
| 2125 | UUUGACACAUUCUUAGCAC | 5023 | GUGCUAAGAAUGUGUCAAA |
| 2126 | UUGACACAUUCUUAGCACU | 5024 | AGUGCUAAGAAUGUGUCAA |
| 2129 | ACACAUUCUUAGCACUGAA | 5027 | UUCAGUGCUAAGAAUGUGU |
| 2131 | ACAUUCUUAGCACUGAACU | 5029 | AGUUCAGUGCUAAGAAUGU |
| 2218 | GCUACUUUGAAGGGAACAA | 5116 | UUGUUCCCUUCAAAGUAGC |
| 2275 | AGUGUCACUUUCUGAGUAC | 5173 | GUACUCAGAAAGUGACACU |
| 2276 | GUGUCACUUUCUGAGUACC | 5174 | GGUACUCAGAAAGUGACAC |
| 2385 | UCACCUGCAUAGUCACUCU | 5283 | AGAGUGACUAUGCAGGUGA |
| 2390 | UGCAUAGUCACUCUUUUGA | 5288 | UCAAAAGAGUGACUAUGCA |
| 2394 | UAGUCACUCUUUUGAUGCU | 5292 | AGCAUCAAAAGAGUGACUA |
| 2448 | GUAUAGCCACAGUUCAUCC | 5346 | GGAUGAACUGUGGCUAUAC |
| 2752 | UAAAUCCCACUCGCUCUUU | 5650 | AAAGAGCGAGUGGGAUUUA |
| 2753 | AAAUCCCACUCGCUCUUUU | 5651 | AAAAGAGCGAGUGGGAUUU |
| 2785 | AUAACUACUGCAUAAUAUG | 5683 | CAUAUUAUGCAGUAGUUAU |
| 2786 | UAACUACUGCAUAAUAUGG | 5684 | CCAUAUUAUGCAGUAGUUA |
| 2787 | AACUACUGCAUAAUAUGGA | 5685 | UCCAUAUUAUGCAGUAGUU |
| 2793 | UGCAUAAUAUGGAUACGCC | 5691 | GGCGUAUCCAUAUUAUGCA |
| 2797 | UAAUAUGGAUACGCCUUAU | 5695 | AUAAGGCGUAUCCAUAUUA |
| 2798 | AAUAUGGAUACGCCUUAUU | 5696 | AAUAAGGCGUAUCCAUAUU |
| 2800 | UAUGGAUACGCCUUAUUUG | 5698 | CAAAUAAGGCGUAUCCAUA |
| 2802 | UGGAUACGCCUUAUUUGAU | 5700 | AUCAAAUAAGGCGUAUCCA |
| 2803 | GGAUACGCCUUAUUUGAUU | 5701 | AAUCAAAUAAGGCGUAUCC |
| 2804 | GAUACGCCUUAUUUGAUUU | 5702 | AAAUCAAAUAAGGCGUAUC |
| 2806 | UACGCCUUAUUUGAUUUAA | 5704 | UUAAAUCAAAUAAGGCGUA |
| 2807 | ACGCCUUAUUUGAUUUAAC | 5705 | GUUAAAUCAAAUAAGGCGU |
| 2808 | CGCCUUAUUUGAUUUAACU | 5706 | AGUUAAAUCAAAUAAGGCG |
| 2811 | CUUAUUUGAUUUAACUAGU | 5709 | ACUAGUUAAAUCAAAUAAG |
| 2817 | UGAUUUAACUAGUUCCCUA | 5715 | UAGGGAACUAGUUAAAUCA |
| 2819 | AUUUAACUAGUUCCCUAAU | 5717 | AUUAGGGAACUAGUUAAAU |
| 2821 | UUAACUAGUUCCCUAAUGA | 5719 | UCAUUAGGGAACUAGUUAA |
| 2825 | CUAGUUCCCUAAUGAUGGA | 5723 | UCCAUCAUUAGGGAACUAG |
| 2826 | UAGUUCCCUAAUGAUGGAC | 5724 | GUCCAUCAUUAGGGAACUA |
| 2827 | AGUUCCCUAAUGAUGGACU | 5725 | AGUCCAUCAUUAGGGAACU |

TABLE 10
Modified siRNA subset F sequences

| SEQ | | SEQ | |
|---|---|---|---|
| ID | | ID | |
| NO: | sense strand sequence (5′-3′) | NO: | antisense strand sequence (5′-3′) |
| 5797 | CfsasGfuUfcAfcAfgCfuGfcCfaAfuAfsusu | 5846 | usAfsuUfgGfcAfgCfuGfuGfaAfcUfgsusu |
| 5798 | AfsgsCfaCfuUfcAfgAfcAfaGfgUfcAfsusu | 5847 | usGfsaCfcUfuGfuCfuGfaAfgUfgCfususu |
| 5799 | CfscsAfgAfgAfcAfcUfgCfgGfaAfuAfsusu | 5848 | usAfsuUfcCfgCfaGfuGfuCfuCfuGfgsusu |
| 5800 | CfsusGfcGfgAfaUfuUfgCfuGfcCfaAfsusu | 5849 | usUfsgGfcAfgCfaAfaUfuCfcGfcAfgsusu |
| 5801 | AfsgsCfcAfgUfuUfuUfaAfgGfgAfcAfsusu | 5850 | usGfsuCfcCfuUfaAfaAfaCfuGfgCfususu |
| 5802 | CfscsAfgUfuUfuUfaAfgGfgAfcAfcAfsusu | 5851 | usGfsuGfuCfcCfuUfaAfaAfaCfuGfgsusu |
| 5803 | CfsasGfuUfuUfuAfaGfgGfaCfaCfcAfsusu | 5852 | usGfsgUfgUfcCfcUfuAfaAfaAfcUfgsusu |
| 5804 | UfsusUfuUfaAfgGfgAfcAfcCfaGfaAfsusu | 5853 | usUfscUfgGfuGfuCfcCfuUfaAfaAfasusu |
| 5805 | UfsusUfaAfgGfgAfcAfcCfaGfaGfcAfsusu | 5854 | usGfscUfcUfgGfuGfuCfcCfuUfaAfasusu |
| 5806 | AfsgsCfcUfgCfuCfuGfaUfuCfuAfuAfsusu | 5855 | usAfsuAfgAfaUfcAfgAfgCfaGfgCfususu |
| 5807 | UfsasAfgAfgUfaAfuUfgCfcUfaAfcAfsusu | 5856 | usGfsuUfaGfgCfaAfuUfaCfuCfuUfasusu |
| 5808 | GfsusAfaUfuGfcCfuAfaCfuUfgAfuAfsusu | 5857 | usAfsuCfaAfgUfuAfgGfcAfaUfuAfcsusu |
| 5809 | UfsasAfuUfgCfcUfaAfcUfuGfaUfuAfsusu | 5858 | usAfsaUfcAfaGfuUfaGfgCfaAfuUfasusu |
| 5810 | UfsusAfaCfcAfaAfcUfuGfuGfgCfcAfsusu | 5859 | usGfsgCfcAfcAfaGfuUfuGfgUfuAfasusu |
| 5811 | GfsasAfuGfaGfuAfaCfuCfcUfgUfcAfsusu | 5860 | usGfsaCfaGfgAfgUfuAfcUfcAfuUfcsusu |
| 5812 | UfsusUfuGfaCfaCfaUfuCfuUfaGfcAfsusu | 5861 | usGfscUfaAfgAfaUfgUfgUfcAfaAfasusu |
| 5813 | UfsusUfgAfcAfcAfuUfcUfuAfgCfaAfsusu | 5862 | usUfsgCfuAfaGfaAfuGfuGfuCfaAfasusu |
| 5814 | UfsusGfaCfaCfaUfuCfuUfaGfcAfcAfsusu | 5863 | usGfsuGfcUfaAfgAfaUfgUfgUfcAfasusu |
| 5815 | AfscsAfcAfuUfcUfuAfgCfaCfuGfaAfsusu | 5864 | usUfscAfgUfgCfuAfaGfaAfuGfuGfususu |
| 5816 | AfscsAfuUfcUfuAfgCfaCfuGfaAfcAfsusu | 5865 | usGfsuUfcAfgUfgCfuAfaGfaAfuGfususu |
| 5817 | GfscsUfaCfuUfuGfaAfgGfgAfaCfaAfsusu | 5866 | usUfsgUfuCfcCfuUfcAfaAfgUfaGfcsusu |
| 5818 | AfsgsUfgUfcAfcUfuUfcUfgAfgUfaAfsusu | 586 | usUfsaCfuCfaGfaAfaGfuGfaCfaCfususu |
| 5819 | GfsusGfuCfaCfuUfuCfuGfaGfuAfcAfsusu | 5868 | usGfsuAfcUfcAfgAfaAfgUfgAfcAfcsusu |
| 5820 | UfscsAfcCfuGfcAfuAfgUfcAfcUfcAfsusu | 5869 | usGfsaGfuGfaCfuAfuGfcAfgGfuGfasusu |
| 5821 | UfsgsCfaUfaGfuCfaCfuCfuUfuUfgAfsusu | 5870 | usCfsaAfaAfgAfgUfgAfcUfaUfgCfasusu |
| 5822 | UfsasGfuCfaCfuCfuUfuUfgAfuGfcAfsusu | 5871 | usGfscAfuCfaAfaAfgAfgUfgAfcUfasusu |
| 5823 | GfsusAfuAfgCfcAfcAfgUfuCfaUfcAfsusu | 5872 | usGfsaUfgAfaCfuGfuGfgCfuAfuAfcsusu |
| 5824 | UfsasAfaUfcCfcAfcUfcGfcUfcUfuAfsusu | 5873 | usAfsaGfaGfcGfaGfuGfgGfaUfuUfasusu |
| 5825 | AfsasAfuCfcCfaCfuCfgCfuCfuUfuAfsusu | 5874 | usAfsaAfgAfgCfgAfgUfgGfgAfuUfususu |
| 5826 | AfsusAfaCfuAfcUfgCfaUfaAfuAfuAfsusu | 5875 | usAfsuAfuUfaUfgCfaGfuAfgUfuAfususu |
| 5827 | UfsasAfcUfaCfuGfcAfuAfaUfaUfgAfsusu | 5876 | usCfsaUfaUfuAfuGfcAfgUfaGfuUfasusu |
| 5828 | AfsasCfuAfcUfgCfaUfaAfuAfuGfgAfsusu | 5877 | usCfscAfuAfuUfaUfgCfaGfuAfgUfususu |
| 5829 | UfsgsCfaUfaAfuAfuGfgAfuAfcGfcAfsusu | 5878 | usGfscGfuAfuCfcAfuAfuUfaUfgCfasusu |
| 5830 | UfsasAfuAfuGfgAfuAfcGfcCfuUfaAfsusu | 5879 | usUfsaAfgGfcGfuAfuCfcAfuAfuUfasusu |
| 5831 | AfsasUfaUfgGfaUfaCfgCfcUfuAfuAfsusu | 5880 | usAfsuAfaGfgCfgUfaUfcCfaUfaUfususu |
| 5832 | UfsasUfgGfaUfaCfgCfcUfuAfuUfuAfsusu | 5881 | usAfsaAfuAfaGfgCfgUfaUfcCfaUfasusu |
| 5833 | UfsgsGfaUfaCfgCfcUfuAfuUfuGfaAfsusu | 5882 | usUfscAfaAfuAfaGfgCfgUfaUfcCfasusu |
| 5834 | GfsgsAfuAfcGfcCfuUfaUfuUfgAfuAfsusu | 5883 | usAfsuCfaAfaUfaAfgGfcGfuAfuCfcsusu |
| 5835 | GfsasUfaCfgCfcUfuAfuUfuGfaUfuAfsusu | 5884 | usAfsaUfcAfaAfuAfaGfgCfgUfaUfcsusu |
| 5836 | UfsasCfgCfcUfuAfuUfuGfaUfuUfaAfsusu | 5885 | usUfsaAfaUfcAfaAfuAfaGfgCfgUfasusu |
| 5837 | AfscsGfcCfuUfaUfuUfgAfuUfuAfaAfsusu | 5886 | usUfsuAfaAfuCfaAfaUfaAfgGfcGfususu |
| 5838 | CfsgsCfcUfuAfuUfuGfaUfuUfaAfcAfsusu | 5887 | usGfsuUfaAfaUfcAfaAfuAfaGfgCfgsusu |
| 5839 | CfsusUfaUfuUfgAfuUfuAfaCfuAfgAfsusu | 5888 | usCfsuAfgUfuAfaAfuCfaAfaUfaAfgsusu |
| 5840 | UfsgsAfuUfuAfaCfuAfgUfuCfcCfuAfsusu | 5889 | usAfsgGfgAfaCfuAfgUfuAfaAfuCfasusu |
| 5841 | AfsusUfuAfaCfuAfgUfuCfcCfuAfaAfsusu | 5890 | usUfsuAfgGfgAfaCfuAfgUfuAfaAfususu |
| 5842 | UfsusAfaCfuAfgUfuCfcCfuAfaUfgAfsusu | 5891 | usCfsaUfuAfgGfgAfaCfuAfgUfuAfasusu |
| 5843 | CfsusAfgUfuCfcCfuAfaUfgAfuGfgAfsusu | 5892 | usCfscAfuCfaUfuAfgGfgAfaCfuAfgsusu |
| 5844 | UfsasGfuUfcCfcUfaAfuGfaUfgGfaAfsusu | 5893 | usUfscCfaUfcAfuUfaGfgGfaAfcUfasusu |
| 5845 | AfsgsUfuCfcCfuAfaUfgAfuGfgAfcAfsusu | 5894 | usGfsuCfcAfuCfaUfuAfgGfgAfaCfususu |

TABLE 11
Alternatively modified siRNA subset F sequences

| | SEQ | | SEQ | |
|---|---|---|---|---|
| siRNA | ID | | ID | |
| Name | NO: | sense strand sequence (5′-3′) | NO: | antisense strand sequence (5′-3′) |
| ETD01314 | 5895 | csasguucAfcAfGfcugccaauasusu | 5846 | usAfsuUfgGfcAfgCfuGfuGfaAfcUfgsusu |
| ETD01315 | 5896 | asgscacuucAfgAfcaaggucasusu | 5847 | usGfsaCfcUfuGfuCfuGfaAfgUfgCfususu |
| ETD01316 | 5897 | cscsagAfgAfcAfcugcggaauasusu | 5848 | usAfsuUfcCfgCfaGfuGfuCfuCfuGfgsusu |
| ETD01317 | 5898 | csusgcggaaUfuUfgcugccaasusu | 5849 | usUfsgGfcAfgCfaAfaUfuCfcGfcAfgsusu |
| ETD01318 | 5899 | asgsccagUfUfUfUfUfaagggacasusu | 5850 | usGfsuCfcCfuUfaAfaAfaCfuGfgCfususu |
| ETD01319 | 5900 | cscsagUfUfUfUfUfaagggacacasusu | 5851 | usGfsuGfuCfcCfuUfaAfaAfaCfuGfgsusu |
| ETD01320 | 5901 | csasguuuuuAfAfggoacaccasusu | 5852 | usGfsgUfgUfcCfcUfuAfaAfaAfcUfgsusu |
| ETD01321 | 5902 | ususuuuaaGfGfGfAfcaccagaasusu | 5853 | usUfscUfgGfuGfuCfcCfuUfaAfaAfasusu |
| ETD01322 | 5903 | ususuaAfgggAfcAfccagagcasusu | 5854 | usGfscUfcUfgGfuGfuCfcCfuUfaAfasusu |
| ETD01323 | 5904 | asgsccUfgCfUfCfUfgauucuauasusu | 5855 | usAfsuAfgAfaUfcAfgAfgCfaGfgCfususu |
| ETD01324 | 5905 | usasagAfguAfAfuugccuaacasusu | 5856 | usGfsuUfaGfgCfaAfuUfaCfuCfuUfasusu |
| ETD01325 | 5906 | gsusaaUfUfgCfCfuaacuugauasusu | 5857 | usAfsuCfaAfgUfuAfgGfcAfaUfuAfcsusu |
| ETD01326 | 5907 | usasauUfgCfcUfaacuugauuasusu | 5858 | usAfsaUfcAfaGfuUfaGfgCfaAfuUfasusu |
| ETD01327 | 5908 | ususaaccAfaAfcuuguggccasusu | 5859 | usGfsgCfcAfcAfaGfuUfuGfgUfuAfasusu |
| ETD01328 | 5909 | gsasaugAfguAfAfcuccugucasusu | 5860 | usGfsaCfaGfgAfgUfuAfcUfcAfuUfcsusu |
| ETD01329 | 5910 | ususuugaCfaCfaUfucuuagcasusu | 5861 | usGfscUfaAfgAfaUfgUfgUfcAfaAfasusu |
| ETD01330 | 5911 | ususugAfcAfcAfuucuuagcaasusu | 5862 | usUfsgCfuAfaGfaAfuGfuGfuCfaAfasusu |
| ETD01331 | 5912 | ususgaCfaCfaUfUfcuuagcacasusu | 5863 | usGfsuGfcUfaAfgAfaUfgUfgUfcAfasusu |
| ETD01332 | 5913 | ascsacaUfUfcUfUfagcacugaasusu | 5864 | usUfscAfgUfgCfuAfaGfaAfuGfuGfususu |
| ETD01333 | 5914 | ascsauucuuAfGfcacugaacasusu | 5865 | usGfsuUfcAfgUfgCfuAfaGfaAfuGfususu |
| ETD01334 | 5915 | gscsuacuuuGfaAfgggaacaasusu | 5866 | usUfsgUfuCfcCfuUfcAfaAfgUfaGfcsusu |
| ETD01335 | 5916 | asgsugUfcacUfuUfcugaguaasusu | 5867 | usUfsaCfuCfaGfaAfaGfuGfaCfaCfususu |
| ETD01336 | 5917 | gsusgucaCfUfUfUfcugaguacasusu | 5868 | usGfsuAfcUfcAfgAfaAfgUfgAfcAfcsusu |
| ETD01337 | 5918 | uscsaccuGfcAfuAfgucacucasusu | 5869 | usGfsaGfuGfaCfuAfuGfcAfgGfuGfasusu |
| ETD01338 | 5919 | usgscaUfagUfCfaCfucuuuugasusu | 5870 | usCfsaAfaAfgAfgUfgAfcUfaUfgCfasusu |
| ETD01339 | 5920 | usasguCfaCfuCfuuuugaugcasusu | 5871 | usGfscAfuCfaAfaAfgAfgUfgAfcUfasusu |
| ETD01340 | 5921 | gsusauAfgccAfcAfguucaucasusu | 5872 | usGfsaUfgAfaCfuGfuGfgCfuAfuAfcsusu |
| ETD01341 | 5922 | usasaaucccAfcucgcucuuasusu | 5873 | usAfsaGfaGfcGfaGfuGfgGfaUfuUfasusu |
| ETD01342 | 5923 | asasauCfcCfaCfuCfgcucuuuasusu | 5874 | usAfsaAfgAfgCfgAfgUfgGfgAfuUfususu |
| ETD01343 | 5924 | asusaaCfUfaCfUfgCfauaauauasusu | 5875 | usAfsuAfuUfaUfgCfaGfuAfgUfuAfususu |
| ETD01344 | 5925 | usasacuAfcuGfcAfuaauaugasusu | 5876 | usCfsaUfaUfuAfuGfcAfgUfaGfuUfasusu |
| ETD01345 | 5926 | asascuaCfUfgCfaUfaauauggasusu | 5877 | usCfscAfuAfuUfaUfgCfaGfuAfgUfususu |
| ETD01346 | 5927 | usgscauAfAfuAfuggauacgcasusu | 5878 | usGfscGfuAfuCfcAfuAfuUfaUfgCfasusu |
| ETD01347 | 5928 | usasauAfuggAfuAfcgccuuaasusu | 5879 | usUfsaAfgGfcGfuAfuCfcAfuAfuUfasusu |
| ETD01348 | 5929 | asasuaUfggaUfaCfgccuuauasusu | 5880 | usAfsuAfaGfgCfgUfaUfcCfaUfaUfususu |
| ETD01349 | 5930 | usasuggaUfaCfgCfcuuauuuasusu | 5881 | usAfsaAfuAfaGfgCfgUfaUfcCfaUfasusu |
| ETD01350 | 5931 | usgsgauaCfgCfCfuuauuugaasusu | 5882 | usUfscAfaAfuAfaGfgCfgUfaUfcCfasusu |
| ETD01351 | 5932 | gsgsauaCfgCfCfuuauuugauasusu | 5883 | usAfsuCfaAfaUfaAfgGfcGfuAfuCfcsusu |
| ETD01352 | 5933 | gsasuaCfgCfCfUfUfauuugauuasusu | 5884 | usAfsaUfcAfaAfuAfaGfgCfgUfaUfcsusu |
| ETD01353 | 5934 | usascgccuuAfuuugauuuaasusu | 5885 | usUfsaAfaUfcAfaAfuAfaGfgCfgUfasusu |
| ETD01354 | 5935 | ascsgccUfUfaUfuUfgauuuaaasusu | 5886 | usUfsuAfaAfuCfaAfaUfaAfgGfcGfususu |
| ETD01355 | 5936 | csgsccUfUfaUfUfugauuuaacasusu | 5887 | usGfsuUfaAfaUfcAfaAfuAfaGfgCfgsusu |
| ETD01356 | 5937 | CsusuauuuGfAfuuuaacuagasusu | 5888 | usCfsuAfgUfuAfaAfuCfaAfaUfaAfgsusu |
| ETD01357 | 5938 | usgsauUfUfaaCfUfaguucccuasusu | 5889 | usAfsgGfgAfaCfuAfgUfuAfaAfuCfasusu |
| ETD01358 | 5939 | asusuUAfAfcuAfguucccuaaasusu | 5890 | usUfsuAfgGfgAfaCfuAfgUfuAfaAfususu |
| ETD01359 | 5940 | ususaacUfagUfUfcccuaaugasusu | 5891 | usCfsaUfuAfgGfgAfaCfuAfgUfuAfasusu |
| ETD01360 | 5941 | csusaguUfCfCfCfuaaugauggasusu | 5892 | usCfscAfuCfaUfuAfgGfgAfaCfuAfgsusu |
| ETD01361 | 5942 | usasguUfcCfCfUfaaugauggaasusu | 5893 | usUfscCfaUfcAfuUfaGfgGfaAfcUfasusu |
| ETD01362 | 5943 | asgsuucccuAfAfugauggacasusu | 5894 | usGfsuCfcAfuCfaUfuAfgGfgAfaCfususu |

Any siRNA among any of subsets A-H may comprise any modification pattern described herein. If a sequence is a different number of nucleotides in length than a modification pattern, the modification pattern may still be used with the appropriate number of additional nucleotides added 5′ or 3′ to match the number of nucleotides in the modification pattern. For example, if a sense or antisense strand of the siRNA among any of subsets A-F comprises 19 nucleotides, and a modification pattern comprises 21 nucleotides. UU may be added onto the 5′ end of the sense or antisense strand.

### Example 4: Screening PLIN1 siRNAs for Activity in Human Cells in Culture

Chemically modified PLIN1 siRNAs cross reactive for human and non-human primate and derived from sequences in siRNA subset F (Table 9) will be assayed for PLIN1 mRNA knockdown activity in cells in culture. UACC-812 (ATCC® CRL-1897™) cells will be seeded in 96-well tissue culture plates at a cell density of 10,000 cells per well in Leibovitz's L-15 Medium (ATCC Catalog No, 30-2008) supplemented with 20% fetal bovine serum and incubated overnight in a water-jacketed, humidified incubator at 37° C. in an atmosphere composed of air. The PLIN1 siRNAs will be individually transfected into UACC-812 cells in duplicate wells at 10 nM final concentration using 0.3 μL Lipofectamine RNAiMax (Fisher) per well. Silencer Select Negative Control #1 (ThermoFisher, Catalog #4390843) will be transfected at 10 nM final concentration as a control. After incubation for 48 hours at 37° C. total RNA will be harvested from each well and cDNA prepared using TaqMan R Fast Advanced Cells-to-CT™ Kit (ThermoFisher. Catalog #A35374) according to the manufacturer's instructions. The level of PLIN1 mRNA from each well will be measured in triplicate by real-time qPCR on a QuantStudio™ 6 Pro Real-Time PCR System using TaqMan Gene Expression Assay for human PLIN1 (ThermoFisher, assay #Hs01106925_m1). The level of PPIA mRNA will be measured using TaqMan Gene Expression Assay (ThermoFisher, assay #Hs99999904_m1) and used to determine relative PLIN1 mRNA levels in each well using the delta-delta Ct method. All data will be normalized to relative PLIN1 mRNA levels in untreated UACC-812 cells.

### Example 5: Determining the IC50 of PLIN1 siRNAs

The IC50 values for knockdown of PLIN1 mRNA by select PLIN1 siRNAs will be determined in UACC-812 (ATCC® CRL-1897™) cells. The siRNAs will be assayed individually at 30 nM, 10 nM, 3 nM, 1 nM and 0.3 nM, or 3 nM, 1 nM, 0.3 nM, 0.1 nM and 0.03 nM, or 30 nM, 10 nM, 3 nM, 1 nM, 0.3 nM, 0.1 nM and 0.03 nM. The UACC-812 cells will be seeded in 96-well tissue culture plates at a cell density of 7,500 cells per well in DMEM (ATCC Catalog No, 30-2002) supplemented with 20% fetal bovine serum and incubated overnight in a water-jacketed, humidified incubator at 37° C. in an atmosphere composed of air plus 5% carbon dioxide. The PLIN1 siRNAs will be individually transfected into UACC-812 cells in triplicate wells using 0.3 μL Lipofectamine RNAiMax (Fisher) per well. After incubation for 48 hours at 37° C. total RNA will be harvested from each well and cDNA prepared using TaqMan R; Fast Advanced Cells-to-CT™ Kit (ThermoFisher. Catalog #A35374) according to the manufacturer's instructions. The level of PLIN1 mRNA from each well will be measured in triplicate by real-time qPCR on a QuantStudio™ 6 Pro Real-Time PCR System using TaqMan Gene Expression Assay for human PLIN1 (ThermoFisher, assay #Hs01106925_m1). The level of PPIA mRNA will be measured using TaqMan Gene Expression Assay (ThermoFisher, assay #Hs99999904_m1) and used to determine relative PLIN1 mRNA levels in each well using the delta-delta Ct method. All data will be normalized to relative PLIN1 mRNA levels in untreated UACC-812 cells. Curve fit will be accomplish using the [inhibitor]vs. response (three parameters) function in GraphPad Prism software.

### Example 6: siRNA-Mediated Knockdown of PLIN1 in HepG2 Cells

siRNAs targeted to PLIN1 mRNA that downregulate levels of PLIN1 mRNA may lead to increased expression of adipose triglyceride lipase (ATGL) mRNA and hormone-sensitive lipase (HSL) mRNA, when administered to the cultured human hepatocellular cell line HEPG2.

On Day 0, the HEPG2 cells are to be seeded at 150,000 cells/mL into a Falcon 24-well tissue culture plate (ThermoFisher Cat. No, 353047) at 0.5 mL per well.

On Day 1, PLIN1 siRNA and negative control siRNA master mixes are prepared. The PLIN1 siRNA master mix contains 350 μL of Opti-MEM (ThermoFisher Cat. No, 4427037—s1288 Lot No. AS02B02D) and 3.5 μL of a mixture of two PLIN1 siRNAs (10 μM stock). The negative control siRNA master mix contains 350 μL of Opti-MEM and 3.5 μL of negative control siRNA (ThermoFisher Cat. No, 4390843, 10 μM stock). Next, 3 μL of TransIT-X2 (Mirus Cat. No. MIR-6000) is added to each master mix. The mixes are incubated for 15 minutes to allow transfection complexes to form, then 51 μL of the appropriate master mix+TransIT-X2 is added to duplicate wells of HEPG2 cells with a final siRNA concentration of 10) nM.

On Day 3, 48 hours post transfection, duplicate wells are lysed using the Cells-to-Ct kit according to the manufacturer's protocol (ThermoFisher Cat. No, 4399002) or using protein lysis buffer containing protease and phosphatase inhibitors. For the Cells-to-Ct, cells are washed with 50 μL using cold 1×PBS and lysed by adding 49.5 μL of Lysis Solution and 0.5 μL DNase 1 per well and pipetting up and down 5 times and incubating for 5 minutes at room temperature. The Stop Solution (5 μL/well) is added to each well and mixed by pipetting up and down five times and incubating at room temperature for 2 minutes. The reverse transcriptase reaction is performed using 22.5 μL of the lysate according to the manufacturer's protocol. Samples are stored at −80° C. until real-time qPCR is performed in triplicate using TaqMan Gene Expression Assays (Applied Biosystems FAM/PLIN1. FAM/ATGL and FAM/HSL and using a BioRad CFX96 Cat. No, 1855195).

A decrease in PLIN1 mRNA expression in the HEPG2 cells is expected after transfection with the PLIN1 siRNAs compared to PLIN1 mRNA levels in HEPG2 cells transfected with the non-specific control siRNA 48 hours after transfection. There is an expected decrease in the amount of ATGL and HSL mRNA. These results will show that the PLIN1 siRNAs elicit knockdown of PLIN1 mRNA in HEPG2 cells and that the decrease in PLIN1 expression is correlated with a decrease in ATGL and HSL mRNAs. This will indicate that beneficial cardiometabolic effects may be obtained upon administration of PLIN1 siRNAs to mammalian subjects such as humans.

### Example 7: ASO-Mediated Knockdown of PLIN1 in HepG2 Cells

ASOs targeted to PLIN1 mRNA that downregulate levels of PLIN1 mRNA may lead to increased expression of adipose triglyceride lipase (ATGL) mRNA and hormone-sensitive lipase (HSL) mRNA, when administered to the cultured human hepatocellular cell line HEPG2.

On Day 0, the HEPG2 cells are to be seeded at 150.000 cells/mL into a Falcon 24-well tissue culture plate (ThermoFisher Cat. No, 353047) at 0.5 mL per well.

On Day 1, PLIN1 ASO and negative control ASO master mixes are prepared. The PLIN1 ASO master mix contains 350 μL of Opti-MEM (ThermoFisher Cat. No, 4427037-s1288 Lot No. AS02B02D) and 3.5 μL of a mixture of two PLIN1 ASOs (10 μM stock). The negative control ASO master mix contains 350 μL of Opti-MEM and 3.5 μL of negative control ASO (ThermoFisher Cat. No, 4390843, 10 μM stock). Next, 3 μL of TransIT-X2 (Mirus Cat. No. MIR-6000) is added to each master mix. The mixes are incubated for 15 minutes to allow transfection complexes to form, then 51 μL of the appropriate master mix+TransIT-X2 is added to duplicate wells of HEPG2 cells with a final ASO concentration of 10 nM.

On Day 3, 48 hours post transfection, duplicate wells are lysed using the Cells-to-Ct kit according to the manufacturer's protocol (ThermoFisher Cat. No, 4399002) or using protein lysis buffer containing protease and phosphatase inhibitors. For the Cells-to-Ct, cells are washed with 50 μL using cold 1×PBS and lysed by adding 49.5 μL of Lysis Solution and 0.5 μL DNase 1 per well and pipetting up and down 5 times and incubating for 5 minutes at room temperature. The Stop Solution (5 μL/well) is added to each well and mixed by pipetting up and down five times and incubating at room temperature for 2 minutes. The reverse transcriptase reaction is performed using 22.5 μL of the lysate according to the manufacturer's protocol. Samples are stored at −80° C. until real-time qPCR is performed in triplicate using TaqMan Gene Expression Assays (Applied Biosystems FAM/PLIN1. FAM/ATGL and FAM/HSL and using a BioRad CFX96 Cat. No, 1855195).

A decrease in PLIN1 mRNA expression in the HEPG2 cells is expected after transfection with the PLIN1 ASOs compared to PLIN1 mRNA levels in HEPG2 cells transfected with the non-specific control ASO 48 hours after transfection. There is an expected decrease in the amount of ATGL and HSL mRNA. These results will show that the PLIN1 ASOs elicit knockdown of PLIN1 mRNA in HEPG2 cells and that the decrease in PLIN1 expression is correlated with a decrease in ATGL and HSL mRNAs. This will indicate that beneficial cardiometabolic effects may be obtained upon administration of PLIN1 ASOs to mammalian subjects such as humans.

### Example 8: Inhibition of PLIN1 in a Mouse Model of Hyperlipidemia Using PLIN1 siRNAs or ASOs

In this experiment, a mouse model of hyperlipidemia and metabolic dysfunction is to be used to evaluate the effect of siRNA or ASO inhibition of PLIN1. The hyperlipidemia and metabolic dysfunction disease model utilizes APOE knockout mice that are fed a high fructose/fat/cholesterol diet to induce hyperlipidemia for 12 weeks prior to treatment. Metabolic status is monitored by measuring fasted serum triglycerides, LDL, HDL, total cholesterol, glucose, ALT, AST and ALP.

Briefly, mice are divided into five groups: Group 1—a group treated with non-targeting control siRNA. Group 2—a group treated with non-targeting control ASO. Group 3—a group treated with PLIN1 siRNA1, Group 4—a group treated with PLIN1 ASO1, Group 5—a group treated with vehicle. Each group contains eight mice (4 males and 4 females).

Administration of siRNA or ASO is achieved with a 200 μL subcutaneous injection of siRNA or ASO resuspended in PBS at concentration of 10 μM. On Study Day 0. Group 1 mice are injected subcutaneously with non-targeting control siRNA. Group 2 mice are injected subcutaneously with non-targeting control ASO. Group 3 mice are injected subcutaneously with siRNA1 targeting mouse PLIN1, Group 4 mice are injected subcutaneously with ASO1 targeting mouse PLIN1, and Group 5 mice are injected subcutaneously with vehicle. Every 7 days after the first injection animals from each group will be dosed. Blood samples are taken twice per week: fasted serum triglycerides. LDL. HDL, total cholesterol, glucose. ALT. AST and ALP are measured.

Six weeks after the start of treatment, the mice are sacrificed by cervical dislocation following an intraperitoneal injection of 0.3 ml Nembutal (5 mg/ml) (Sigma Cat. No, 1507002). Final blood samples are collected, and livers are removed and a section placed in RNAlater for mRNA isolation.

mRNA is isolated from tissue placed in RNAlater solution using the PureLink kit according to the manufacturer's protocol (ThermoFisher Cat. No, 12183020). The reverse transcriptase reaction is performed according to the manufacturer's protocol. Samples are stored at −80° C. until real-time qPCR is performed in triplicate using TaqMan Gene Expression Assays (Applied Biosystems FAM/PLIN1 using a BioRad CFX96 Cat. No, 1855195).

A decrease in PLIN1 mRNA expression in the liver tissue from mice dosed with the PLIN1 siRNA1 or ASO1 is expected compared to PLIN1 mRNA levels in the liver tissue from mice dosed with the non-specific controls. There is an expected decrease in fasted serum triglycerides and an increase in fasted serum HDL in mice that receive the PLIN1 siRNA or ASO compared to the mice that receive the non-specific control. These results will show that the PLIN1 siRNA or ASO elicit knockdown of PLIN1 mRNA in liver tissue and that the decrease in PLIN1 expression is correlated with a decrease in fasted serum triglyceride and an increase in fasted serum HDL. These results will further indicate that beneficial cardiometabolic effects may be obtained upon administration of PLIN1 ASOs to primate subjects such as humans.

### Example 9: siRNA-Mediated Knockdown of PLIN1 in Adipose Tissue in a Mice

4-7 week old ICR mice (Envigo Labs) mice in Group 1 (n=4) were given 100 μL of phosphate buffered saline (PBS) or given 500 μg of siRNA targeting mouse PLIN1 in 100 μL PBS by a single subcutaneous injection. The siRNA duplexes are depicted in Table 12, and each included a lipid moiety. In the table, Nf (e.g. Af, Cf, Gf, Tf, or Uf) is a 2′ fluoro-modified nucleoside, n (e.g. a, c, g, t, or u) is a 2′ O-methyl modified nucleoside, “s” is a phosphorothioate linkage, and “5Vp” is a 5′ vinylphosphonate. On Day 20, the mice were then euthanized and an abdominal white fat sample from each was collected and placed in RNAlater (ThermoFisher Cat #AM7020) until processing. Total fat RNA was prepared by homogenizing the tissue in homogenization buffer (Maxwell RSC simplyRNA Tissue Kit) using a Percellys 24 tissue homogenizer (Bertin Instruments) set at 5000 rpm for two 10 second cycles. The homogenate was centrifuged for 10′ at 16,000×g at 4C and the lower liquid layer was removed to a fresh tube. The sample was centrifuged two additional times, each time removing the lower liquid layer to a fresh tube. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of mouse PLIN1 mRNA were assessed by RT-qPCR in triplicate on a QuantStudio™ 6 Pro Real-Time PCR System using TaqMan assays for mouse PLIN1 (ThermoFisher, assay #Mm00558672_m1) and the mouse housekeeping gene PPIA (ThermoFisher, assay #Mm02342430_g1). Data were normalized to the level in animals receiving PBS.

TABLE 12
Example siRNA duplexes

| | | Sense Strand | | |
|---|---|---|---|---|
| | | Sequence | Anti- | |
| | Sense | (5′ to 3′) | sense | Antisense |
| | SEQ | with 5′ | SEQ | Strand |
| SIRNA | ID | Hydrophobic | ID | Sequence |
| Name | NO: | Moiety | NO: | (5′ to 3′) |
| ETD01540 | 5944 | [ETL3]CfsaAfc | 5957 | usUfsgUfcGf |
| | | AfcUfcUfuUfcU | | aGfaAfaGfa |
| | | fcGfaCfaAfsu | | GfUGfUUfgs |
| | | su | | usu |
| ETD01624 | 5944 | [ETL3]CfsaAfc | 5958 | 5VpusUfsgUfc |
| | | AfcUfcUfuUfcU | | GfaGfaAfaGfa |
| | | fcGfaCfaAfsu | | GfuGfUUfgsu |
| | | su | | su |
| ETD01751 | 5945 | [ETL3]CfaAfc | 5958 | 5VpusUfsgUf |
| | | AfcUfcUfuUfc | | cGfaGfaAfa |
| | | UfcGfaCfaAfs | | GfaGfuGfUU |
| | | usu | | fgsusu |

The results are depicted in Table 13. Addition of 5′ vinylphosphonate (5Vp) resulted in increased potency and enabled delivery to adipose tissue.

TABLE 13
mRNA expression levels

| | Average mRNA relative to |
|---|---|
| siRNA | no treatment control animals |
| ETD01540 | 1.7 |
| ETD01624 | 0.67 |
| ETD01751 | 0.53 |

### Example 10: Knockdown of PLIN1 in Adipose Tissue of Mice Using Cholesterol-Conjugated SiRNA

4-6 week old ICR mice (Envigo Labs) mice in Group 1 (n=4) were given 100 μL of phosphate buffered saline (PBS) or 1.5 mg of a cholesterol-conjugated siRNA, ETD01510, targeting mouse PLIN1 in 100 μL PBS by subcutaneous injection. A separate group of mice were given ETD01510 by intravenous injection. The siRNA duplex ETD01510 is depicted in Table 14, and included a hydrophobic moiety. In the table, Nf (e.g. Af, Cf, Gf, Tf, or Uf) is a 2′ fluoro-modified nucleoside, n (e.g. a, c, g, t, or u) is a 2′ O-methyl modified nucleoside, and “s” is a phosphorothioate linkage. On Day 9, the mice were then euthanized and an abdominal white fat sample from each was collected and placed in RNAlater (ThermoFisher Cat #AM7020) until processing. Total fat RNA was prepared by homogenizing the tissue in homogenization buffer (Maxwell RSC simplyRNA Tissue Kit) using a Percellys 24 tissue homogenizer (Bertin Instruments) set at 5000 rpm for two 10 second cycles. The homogenate was centrifuged for 10′ at 16,000×g at 4C and the liquid layer was removed to a fresh tube. The sample was centrifuged two additional times, each time removing the lower liquid layer to a fresh tube. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of mouse PLIN1 mRNA were assessed by RT-qPCR in triplicate on a QuantStudio™ 6 Pro Real-Time PCR System using TaqMan assays for mouse PLIN1 (ThermoFisher, assay #Mm00558672_m1) and the mouse housekeeping gene PPIA (ThermoFisher, assay #Mm02342430_g1). Data were normalized to the level in animals receiving PBS. The results are shown in Table 15. The cholesterol-conjugated siRNA ETD01510 reduced PLIN1 mRNA in adipose tissue in mice.

TABLE 14
Example siRNA Sequences

| | | Sense | | |
|---|---|---|---|---|
| | | Strand | | |
| | | Sequence | | |
| | | (5′ to 3′) | Anti- | |
| | Sense | with 5′ | sense | Antisense |
| | SEQ | Hydro- | SEQ | Strand |
| SiRNA | ID | phobic | ID | Sequence |
| Name | NO: | Moiety | NO: | (5′ to 3′) |
| ETD01510 | 5946 | [ETL2]Cfsa | 5959 | usUfsgUfcG |
| | | sAfcAfcUfc | | faGfaAfaGf |
| | | UfuUfcUfcG | | aGfuGfuUfg |
| | | faCfaAfsus | | susu |
| | | u | | |

TABLE 15
Relative PLIN1 mRNA Levels in Adipose Tissue of Mice

| | | | | | Mean PLIN1 mRNA |
|---|---|---|---|---|---|
| | | | Dose | | (Normalized to |
| Group | n | Treatment | (mg) | Route | Group 1, Day 9) |
| 1 | 3 | PBS | 0 | SQ | 1.00 |
| 2 | 3 | ETD01510 | 1.5 | SQ | 0.35 |
| 3 | 3 | ETD01510 | 1.5 | IV | 0.64 |

SQ, subcutaneous;
IV, intravenous

### Example 11: Screening of siRNAs Targeting Human PLIN1 mRNA in Mice Transfected with AAV8-TBG-h-PLIN1

Several siRNAs designed to be cross-reactive with human and cynomolgus monkey PLIN1 mRNA were tested for activity in mice following transfection with an adeno-associated viral vector. The siRNAs were attached to the GalNAc ligand ETL1. The siRNA sequences are shown in Table 16, where “Nf” is a 2′ fluoro-modified nucleoside, “n” is a 2′ O-methyl modified nucleoside, and “s” is a phosphorothioate linkage.

Six to eight week old female mice (C57Bl/6) were injected with 5 μL of a recombinant adeno-associated virus 8 (AAV8) vector (1.8×10E¹³ genome copies/mL) by the retroorbital route on Day −14. The recombinant AAV8 contained the sequence of the human PLIN1 (NM_002666.5) under the control of the human thyroxine binding globulin promoter in an AAV2 backbone packaged in AAV8 capsid (AAV8-TBG-h-PLIN1). On Day 0, infected mice (n=5) were given a subcutaneous injection of a single 100 μg dose of a GalNAc-conjugated siRNA or PBS as vehicle control.

Mice were euthanized on Day 10 after subcutaneous injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver PLIN1 mRNA were assessed by RT-qPCR in triplicate on a QuantStudio™ 6 Pro Real-Time PCR System using TaqMan assays for human PLIN1 (ThermoFisher, assay #Hs01106925_m1) and the mouse housekeeping gene PPIA (ThermoFisher, assay #Mm02342430_g1) and PerfeCTa® qPCR FastMix®, Low ROX™ (VWR, Catalog #101419-222). Data were normalized to the mean PLIN1 mRNA level in animals receiving PBS. Results are shown in Table 17. Mice injected with ETD01754, ETD01755 or ETD01756 had reduced mean liver PLIN1 mRNA on Day 10 relative to mice receiving PBS.

TABLE 16
Example siRNA Sequences

| | | | Anti- | Anti- |
|---|---|---|---|---|
| | Sense | Sense Strand | sense | sense |
| | SEQ | Sequence | SEQ | Strand |
| SIRNA | ID | (5′-3′) with | ID | Sequence |
| Name | NO: | GalNAc moiety | NO: | (5′-3′) |
| ETD01754 | 5947 | [ETL1]suaagAf | 5960 | usGfsUUfaGf |
| | | GfUAfAfuugccu | | gCfaAfuUfaC |
| | | aacasusu | | fuCfuUfasus |
| | | | | u |
| ETD01755 | 5948 | [ETL1]sguaaUf | 5961 | usAfsuCfaAf |
| | | UfgcCfUfaacuu | | gUfuAfgGfcA |
| | | gauasusu | | faUfUAfcsus |
| | | | | u |
| ETD01756 | 5949 | [ETL1]suuugAf | 5962 | usUfsgCfuAf |
| | | cAfcAfuucuuag | | aGfaAfuGfUG |
| | | caasusu | | fuCfaAfasus |
| | | | | u |

TABLE 17
Relative human PLIN1 mRNA Levels in Livers of Mice

| | | | | Mean PLIN1 mRNA |
|---|---|---|---|---|
| | | | Dose | (Normalized to |
| Group | n | Treatment | (ug) | Group 1, Day 10) |
| 1 | 5 | PBS | 0 | 1.00 |
| 2 | 5 | ETD01754 | 100 | 0.12 |
| 3 | 5 | ETD01755 | 100 | 0.12 |
| 4 | 5 | ETD01756 | 100 | 0.17 |

### Example 12: Screening of Additional siRNAs Targeting Human PLIN1 mRNA in Mice Transfected with AAV8-TBG-h-PLIN1

Additional siRNAs designed to be cross-reactive with human and cynomolgus monkey PLIN1 mRNA were tested for activity in mice following transfection with an adeno-associated viral vector. The siRNAs were attached to the GalNAc ligand ETL1 or ETL17. The siRNA sequences are shown in Table 18, where “Nf” is a 2′ fluoro-modified nucleoside, “n” is a 2′ O-methyl modified nucleoside, and “s” is a phosphorothioate linkage.

Six to eight week old female mice (C57Bl/6) were injected with 10 μL of a recombinant adeno-associated virus 8 (AAV8) vector (1.7×10E¹³ genome copies/mL) by the retroorbital route on Day-14. The recombinant AAV8 contained the sequence of the human PLIN1 (NM_002666.5) under the control of the human thyroxine binding globulin promoter in an AAV2 backbone packaged in AAV8 capsid (AAV8-TBG-h-PLIN1). On Day 0, infected mice (n=4) were given a subcutaneous injection of a single 100 μg dose of a GalNAc-conjugated siRNA or PBS as vehicle control.

Mice were euthanized on Day 10 after subcutaneous injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver PLIN1 mRNA were assessed by RT-qPCR in triplicate on a QuantStudio™ 6 Pro Real-Time PCR System using TaqMan assays for human PLIN1 (ThermoFisher, assay #Hs01106925_m1) and the mouse housekeeping gene PPIA (ThermoFisher, assay #Mm02342430_g1) and PerfeCTa® qPCR FastMix®, Low ROX™ (VWR, Catalog #101419-222). Data were normalized to the mean PLIN1 mRNA level in animals receiving PBS. Results are shown in Table 19. All of the siRNAs tested caused a reduction in mean liver PLIN1 mRNA on Day 10 relative to mice receiving PBS. The siRNAs ETD01900, ETD01901 and ETD01902 gave the largest reductions in mean liver PLIN1 mRNA.

TABLE 18
Example siRNA Sequences

| | | Sense | | |
|---|---|---|---|---|
| | | Strand | | |
| | | Sequence | | Anti- |
| | Sense | (5′-3′) | Anti- | sense |
| | SEQ | with | sense | Strand |
| SIRNA | ID | GalNAc | SEQ ID | Sequence |
| Name | NO: | moiety | NO: | (5′-3′) |
| ETD01754 | 5947 | [ETL1]suaagAf | 5960 | usGfsUU |
| | | GfUAfAf | | faGfgCf |
| | | uugccua | | aAfuUfa |
| | | acasusu | | CfuCfuU |
| | | | | fasusu |
| ETD01899 | 5950 | [ETL17]scaguuu | 5963 | usGfsgU |
| | | uuAfaGf | | fgUfcCf |
| | | ggacacc | | cUfuAfa |
| | | asusu | | AfaAfcU |
| | | | | fgsusu |
| ETD01900 | 5951 | ETL17]suuuuuA | 5964 | usUfscU |
| | | fAfGfGf | | fgGfuGf |
| | | gAfcacc | | uCfcCfu |
| | | agaasus | | UfaAfaA |
| | | u | | fasusu |
| ETD01901 | 5952 | [ETL17]suuuUga | 5965 | usGfscU |
| | | CfaCfaU | | faAfgAf |
| | | fucuuag | | aUfgUfg |
| | | casusu | | UfcAfaA |
| | | | | fasusu |
| ETD01902 | 5953 | ETL17]suugaCf | 5966 | usGfsuG |
| | | aCfaUfu | | fcUfaAf |
| | | Cfuuagc | | gAfaUfg |
| | | acasusu | | UfgUfcA |
| | | | | fasusu |
| ETD01903 | 5954 | [ETL17]sacauuc | 5967 | usGfsUU |
| | | uuAfGfc | | fcAfgUf |
| | | acugaac | | gCfuAfa |
| | | asusu | | GfaAfUG |
| | | | | fususu |
| ETD01904 | 5955 | ETL17]sugcaUf | 5968 | usCfsaA |
| | | agUfCfa | | faAfgAf |
| | | Cfucuuu | | gUfgAfc |
| | | ugasusu | | UfaUfgC |
| | | | | fasusu |
| ETD01905 | 5956 | [ETL17]s | 5969 | usCfscA |
| | | aacuaCf | | fuAfuUf |
| | | UfgCfaU | | aUfgCfa |
| | | faauaug | | GfUAfgU |
| | | gasusu | | fususu |

TABLE 19
Relative human PLIN1 mRNA Levels in Livers of Mice

| | | | | Mean PLIN1 mRNA |
|---|---|---|---|---|
| | | | Dose | (Normalized to |
| Group | n | Treatment | (ug) | Group 1, Day 10) |
| 1 | 4 | PBS | 0 | 1.00 |
| 2 | 4 | ETD01754 | 100 | 0.44 |
| 3 | 4 | ETD01899 | 100 | 0.79 |
| 4 | 4 | ETD01900 | 100 | 0.30 |
| 5 | 4 | ETD01901 | 100 | 0.37 |
| 6 | 4 | ETD01902 | 100 | 0.24 |
| 7 | 4 | ETD01903 | 100 | 0.83 |
| 8 | 4 | ETD01904 | 100 | 0.58 |

### Example 13: Oligonucleotide Synthesis

Oligonucleotides such as siRNAs may be synthesized according to phosphoramidite technology on a solid phase. For example, a K & A oligonucleotide synthesizer may be used. Syntheses may be performed on a solid support made of controlled pore glass (CPG, 500 Å or 600 Å, obtained from AM Chemicals, Oceanside, CA, USA). All 2′-OMe and 2′-F phosphoramidites may be purchased from Hongene Biotech (Union City, CA, USA). All phosphoramidites may be dissolved in anhydrous acetonitrile (100 mM) and molecular sieves (3 Å) may be added, 5-Benzylthio-1H-tetrazole (BTT, 250 mM in acetonitrile) or 5-Ethylthio-1H-tetrazole (ETT, 250 mM in acetonitrile) may be used as activator solution. Coupling times may be 9-18 min (e.g, with a GalNAc such as ETL17), 6 min (e.g, with 2′OMe and 2′F). In order to introduce phosphorothioate linkages, a 100 mM solution of 3-phenyl 1,2,4-dithiazoline-5-one (POS, obtained from Poly Org, Inc., Leominster, Mass., USA) in anhydrous acetonitrile may be employed.

After solid phase synthesis, the dried solid support may be treated with a 1:1 volume solution of 40 wt. % methylamine in water and 28% ammonium hydroxide solution (Aldrich) for two hours at 30° C. The solution may be evaporated and the solid residue may be reconstituted in water and purified by anionic exchange HPLC using a TKSgel SuperQ-5PW 13u column. Buffer A may be 20 mM Tris, 5 mM EDTA, pH 9.0 and contained 20% Acetonitrile and buffer B may be the same as buffer A with the addition of 1 M sodium chloride. UV traces at 260 nm may be recorded. Appropriate fractions may be pooled then desalted using Sephadex G-25 medium.

Equimolar amounts of sense and antisense strand may be combined to prepare a duplex. The duplex solution may be prepared in 0.1×PBS (Phosphate-Buffered Saline, 1×, Gibco) The duplex solution may be annealed at 95° C., for 5 min, and cooled to room temperature slowly. Duplex concentration may be determined by measuring the solution absorbance on a UV-Vis spectrometer at 260 nm in 0.1×PBS. For some experiments, a conversion factor may be calculated from an experimentally determined extinction coefficient.

### Example 14: GalNAc Ligand for Hepatocyte Targeting of Oligonucleotides

Without limiting the disclosure to these individual methods, there are at least two general methods for attachment of multivalent N-acetylgalactosamine (GalNAc) ligands to oligonucleotides: solid or solution-phase conjugations. GalNAc ligands may be attached to solid phase resin for 3″ conjugation or at the 5′ terminus using GalNAc phosphoramidite reagents. GalNAc phosphoramidites may be coupled on solid phase as for other nucleosides in the oligonucleotide sequence at any position in the sequence. Reagents for GalNAc conjugation to oligonucleotides are shown in Table 20.

TABLE 20
GalNAc Conjugation Reagents

| Type of | |
|---|---|
| conjugation | Structure |
| Solid phase 3′ attachment where squiggly line is rest of oligonucleotide chain and right-most OH is where attachment\grave{ } to solid phase is. | 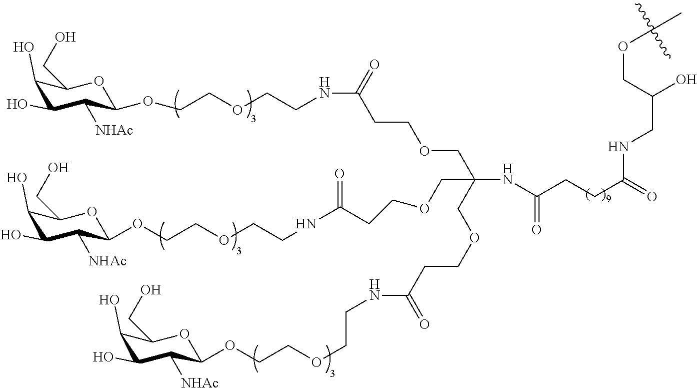 |
| | This GalNAc ligand may be referred to as “GalNAc23” or “GalNAc#23.” |
| Solid phase 5′ attachment phosphoramidite | 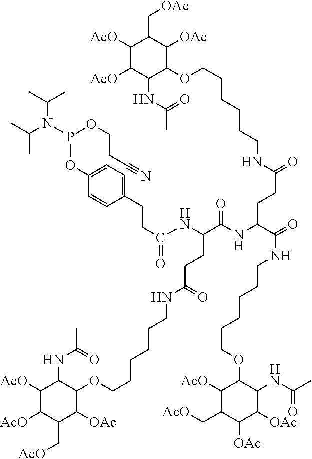 [SMILES: CC(=O)NC1C(OCCCCCCNC(=O)CCC(NC(=O)CCc2ccc(OP(OCCC#N)N(C(C)C)C(C)C)cc2)C(=O)NC(CCC(=O)NCCCCCCOC2C(NC(C)=O)C(OC(C)=O)C(OC(C)=O)C(COC(C)=O)C2OC(C)=O)C(=O)NCCCCCCOC2C(NC(C)=O)C(OC(C)=O)C(OC(C)=O)C(COC(C)=O)C2OC(C)=O)C(OC(C)=O)C(COC(C)=O)C(OC(C)=O)C1OC(C)=O] |
| Solid phase 5′ attachment Phosphoramidite | 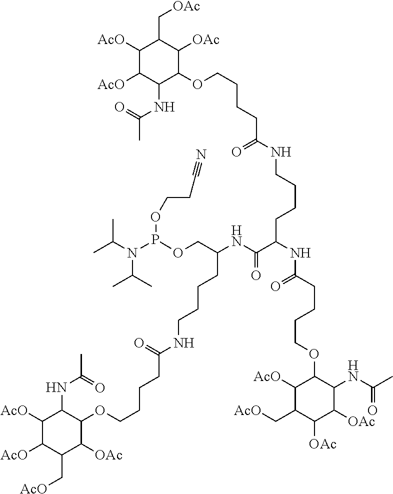 [SMILES: CC(=O)NC1C(OCCCCC(=O)NCCCCC(COP(OCCC#N)N(C(C)C)C(C)C)NC(=O)C(CCCCNC(=O)CCCCOC2C(NC(C)=O)C(OC(C)=O)C(OC(C)=O)C(COC(C)=O)C2OC(C)=O)NC(=O)CCCCOC2C(NC(C)=O)C(OC(C)=O)C(OC(C)=O)C(COC(C)=O)C2OC(C)=O)C(OC(C)=O)C(COC(C)=O)C(OC(C)=O)C1OC(C)=O] |
| Solution phase Carboxylic acid for amide coupling anywhere on oligonucleotide | 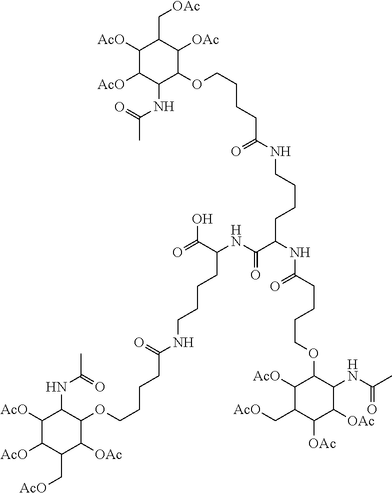 [SMILES: CC(=O)NC1C(OCCCCC(=O)NCCCCC(NC(=O)C(CCCCNC(=O)CCCCOC2C(NC(C)=O)C(OC(C)=O)C(OC(C)=O)C(COC(C)=O)C2OC(C)=O)NC(=O)CCCCOC2C(NC(C)=O)C(OC(C)=O)C(OC(C)=O)C(COC(C)=O)C2OC(C)=O)C(=O)O)C(OC(C)=O)C(COC(C)=O)C(OC(C)=O)C1OC(C)=O] |
| | Where Ac is an acetyl group or other hydroxyl protecting group that can be removed |
| | under basic, acid or reducing conditions. |

In solution phase conjugation, the oligonucleotide sequence—including a reactive conjugation site—is formed on the resin. The oligonucleotide is then removed from the resin and GalNAc is conjugated to the reactive site.

The carboxy GalNAc derivatives may be coupled to amino-modified oligonucleotides. The peptide coupling conditions are known to the skilled in the art using a carbodiimide coupling agent like DCC (N,N′-Dicyclohexylcarbodiimide). EDC (N-(3-dimethylaminopropyl)-N′-ethylcarbodiimide) or EDC.HCl (N-(3-dimethylaminopropyl)-N′-ethylcarbodiimide hydrochloride and an additive like HOBt (1-hydroxy benztriazole). HOSu (N-hydroxysuccinimide). TBTU (N,N,N′,N′-Tetramethyl-O-(benzotriazol-1-yl)uronium tetrafluoroborate. HBTU (2-(1H-benzotriazol-1-yl)-1,1,3,3-tetramethyluronium hexafluorophosphate) or HOAt (1-Hydroxy-7-azabenzotriazole and common combinations thereof such as TBTU/HOBt or HBTU/HOAt to form activated amine-reactive esters.

Amine groups may be incorporated into oligonucleotides using a number of known, commercially available reagents at the 5′ terminus, 3′ terminus or anywhere in between.

Non-limiting examples of reagents for oligonucleotide synthesis to incorporate an amino group include:
5′ attachment:
6-(4-Monomethoxytritylamino)hexyl-(2-cyanoethyl)-(N,N-diisopropyl)-phosphoramidite CAS Number: 114616-27-2
5′-Amino-Modifier TEG CE-Phosphoramidite
10-(O-trifluoroacetamido-N-ethyl)-triethyleneglycol-1-[(2-cyanoethyl)-(N,N-diisopropyl)]-phosphoramidite
3′ attachment:
3′-Amino-Modifier Serinol CPG
3-Dimethoxytrityloxy-2-(3-(fluorenylmethoxy carbonylamino)propanamido)propyl-1-O-succinyl-long chain alkylamino-CPG (where CPG stands for controlled-pore glass and is the solid support)
Amino-Modifier Serinol Phosphoramidite
3-Dimethoxytrityloxy-2-(3-(fluorenylmethoxy carbonylamino)propanamido)propyl-1-O-(2-cyanoethyl)-(N,N-diisopropyl)-phosphoramidite

Internal (base modified):
Amino-Modifier C6 dT
5′-Dimethoxytrityl-5-[N-(trifluoracetylaminohexyl)-3-acrylamido]-2′-deoxyUridine,3′-[(2-cyanoethyl)-(N,N-diisopropyl)]-phosphoramidite. CAS Number: 178925-21-8

Solution phase conjugations may occur after oligonucleotide synthesis via reactions between non-nucleosidic nucleophilic functional groups that are attached to the oligonucleotide and electrophilic GalNAc reagents. Examples of nucleophilic groups include amines and thiols, and examples of electrophilic reagents include activated esters (e.g. N-hydroxysuccinimide, pentafluorophenyl) and maleimides.

### Example 15: GalNAc Ligands for Hepatocyte Targeting of Oligonucleotides

Without limiting the disclosure to these individual methods, there are at least two general methods for attachment of multivalent N-acetylgalactosamine (GalNAc) ligands to oligonucleotides: solid or solution-phase conjugations. GalNAc ligands may be attached to solid phase resin for 3″ conjugation or at the 5′ terminus using GalNAc phosphoramidite reagents. GalNAc phosphoramidites may be coupled on solid phase as for other nucleosides in the oligonucleotide sequence at any position in the sequence. A non-limiting example of a phosphoramidite reagent for GalNAc conjugation to a 5′ end oligonucleotide is shown in Table 21.

TABLE 21
GalNAc Conjugation Reagent

| Type of | |
|---|---|
| conjugation | Structure |
| Solid phase 5′ attachment phosphor- amidite | 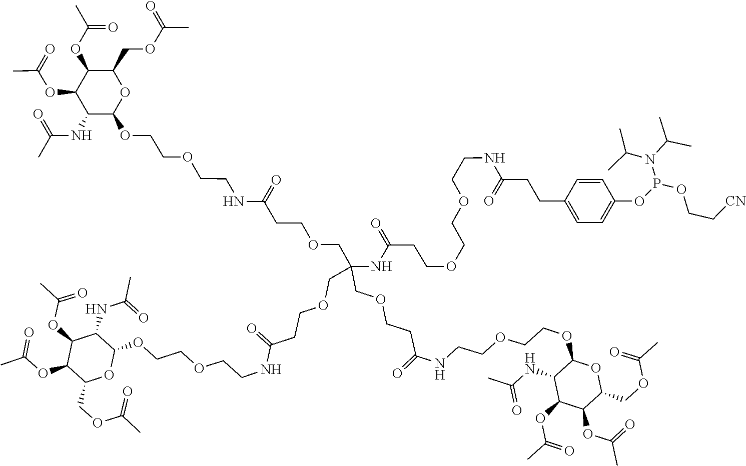 [SMILES: CC(=O)N[C@@H]1[C@H](OCCOCCNC(=O)CCOCC(COCCC(=O)NCCOCCO[C@H]2O[C@H](COC(C)=O)[C@@H](OC(C)=O)[C@@H](OC(C)=O)[C@@H]2NC(C)=O)(COCCC(=O)NCCOCCO[C@@H]2O[C@H](COC(C)=O)[C@H](OC(C)=O)[C@H](OC(C)=O)[C@H]2NC(C)=O)NC(=O)CCOCCOCCNC(=O)CCc2ccc(OP(OCCC#N)N(C(C)C)C(C)C)cc2)O[C@H](COC(C)=O)[C@@H](OC(C)=O)[C@H]1OC(C)=O] |

The following includes examples of synthesis reactions used to create a GalNAc moiety: Scheme for the preparation of NAcegal-Linker-TMSOTf

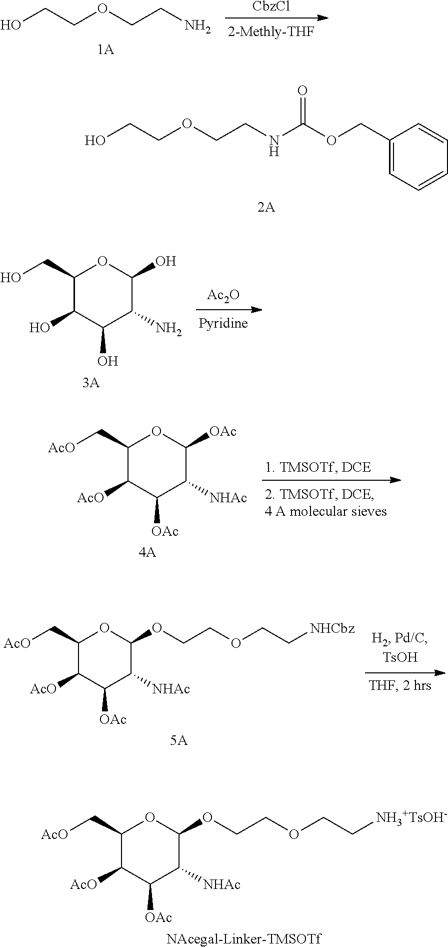

### General Procedure for Preparation of Compound 2A

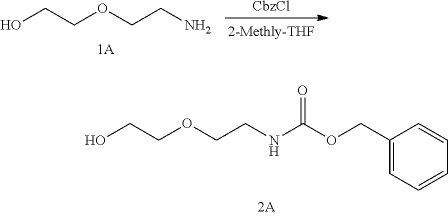

To a solution of Compound 1A (500 g, 4.76 mol, 476 mL) in 2-Methyl-THF (2.00 L) is added CbzCl (406 g, 2.38 mol, 338 mL) in 2-Methyl-THF (750 mL) dropwise at 0° C. The mixture is stirred at 25° C., for 2 hrs under N₂ atmosphere. TLC (DCM:MeOH=20:1, PMA) may indicate CbzCl is consumed completely and one new spot (R_{f}=0.43) formed. The reaction mixture is added HCl/EtOAc (1 N, 180 mL) and stirred for 30 mins, white solid is removed by filtration through celite, the filtrate is concentrated under vacuum to give Compound 2A (540 g, 2.26 mol, 47.5% yield) as a pale yellow oil and used into the next step without further purification, ¹HNMR: δ 7.28-7.41 (m, 5H), 5.55 (br s, 1H), 5.01-5.22 (m, 2H), 3.63-3.80 (m, 2H), 3.46-3.59 (m, 4H), 3.29-3.44 (m, 2H), 2.83-3.02 (m, 1H).

### General Procedure for Preparation of Compound 4A

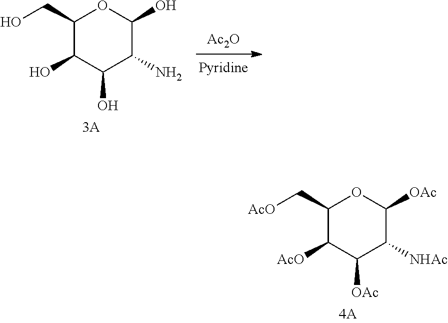 [SMILES: "Ac2O" ; N[C@@H]1[C@@H](O)[C@@H](O)[C@@H](CO)O[C@H]1O ; CC(=O)N[C@H]1[C@H](OC(C)=O)O[C@H](COC(C)=O)[C@H](OC(C)=O)[C@@H]1OC(C)=O]

To a solution of Compound 3A (1.00 kg, 4.64 mol, HCl) in pyridine (5.00 L) is added acetyl acetate (4.73 kg, 46.4 mol, 4.34 L) dropwise at 0° C. under N₂ atmosphere. The mixture is stirred at 25° C., for 16 hrs under N₂ atmosphere. TLC (DCM:MeOH=20:1, PMA) indicated Compound 3A is consumed completely and two new spots (R_{f}=0.35) formed. The reaction mixture is added to cold water (30.0 L) and stirred at 0° C., for 0.5 hr, white solid formed, filtered and dried to give Compound 4A (1.55 kg, 3.98 mol, 85.8% yield) as a white solid and used in the next step without further purification, ¹H NMR: δ 7.90 (d, J=9.29 Hz, 1H), 5.64 (d, J=8.78 Hz, 1H), 5.26 (d, J=3.01 Hz, 1H), 5.06 (dd, J=11.29, 3.26 Hz, 1H), 4.22 (t, J=6.15 Hz, 1H), 3.95-4.16 (m, 3H), 2.12 (s, 3H), 2.03 (s, 3H), 1.99 (s, 3H), 1.90 (s, 3H), 1.78 (s, 3H).

### General Procedure for Preparation of Compound 5A

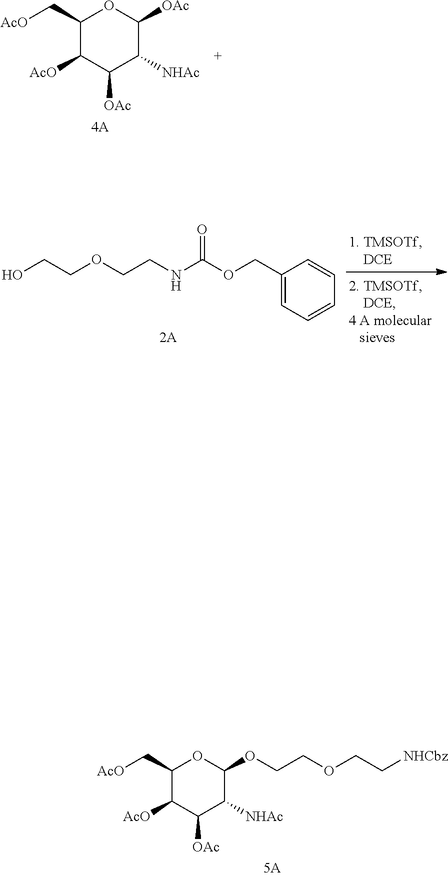 [SMILES: CC(=O)N[C@H]1[C@H](OC(C)=O)O[C@H](COC(C)=O)[C@H](OC(C)=O)[C@@H]1OC(C)=O ; O=C(NCCOCCO)OCc1ccccc1 ; CC(=O)N[C@H]1[C@H](OCCOCCNC(=O)OCc2ccccc2)O[C@H](COC(C)=O)[C@H](OC(C)=O)[C@@H]1OC(C)=O]

To a solution of Compound 4A (300 g, 771 mmol) in DCE (1.50 L) is added TMSOTf (257 g 1.16 mol, 209 mL) and stirred for 2 hrs at 60° C., and then stirred for 1 hr at 25° C. Compound 2A (203 g, 848 mmol) is dissolved in DCE (1.50 L) and added 4 Å powder molecular sieves (150 g) stirring for 30 mins under N₂ atmosphere. Then the solution of Compound 4A in DCE is added dropwise to the mixture at 0° C. The mixture is stirred at 25° C., for 16 hrs under N₂ atmosphere. TLC (DCM:MeOH=25:1, PMA) indicated Compound 4A is consumed completely and new spot (R_{f}=0.24) formed. The reaction mixture is filtered and washed with sat. NaHCO₃ (2.00 L), water (2.00 L) and sat. brine (2.00 L). The organic layer is dried over anhydrous Na₂SO4, filtered and concentrated under reduced pressure to give a residue. The residue is triturated with 2-Me-THE/heptane (5/3, v/v, 1.80 L) for 2 hrs, filtered and dried to give Compound 5A (225 g, 389 mmol, 50.3% yield, 98.4% purity) as a white solid, ¹H NMR: δ 7.81 (d, J=9.29 Hz, 1H), 7.20-7.42 (m, 6H), 5.21 (d, J=3.26 Hz, 1H), 4.92-5.05 (m, 3H), 4.55 (d, J=8.28 Hz, 1H), 3.98-4.07 (m, 3H), 3.82-3.93 (m, 1H), 3.71-3.81 (m, 1H), 3.55-3.62 (m, 1H), 3.43-3.53 (m, 2H), 3.37-3.43 (m, 2H), 3.14 (q, J=5.77 Hz, 2H), 2.10 (s, 3H), 1.99 (s, 3H), 1.89 (s, 3H), 1.77 (s, 3H).

### General Procedure for Preparation of NAcegal-Linker-Tosylate Salt

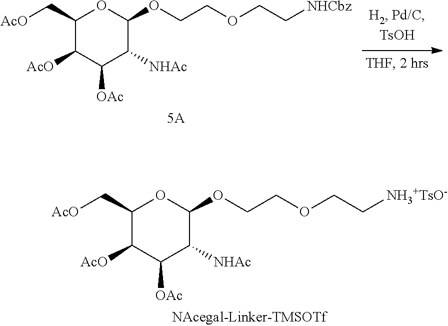

To a solution of Compound 5A (200 g, 352 mmol) in THF (1.0 L) is added dry Pd/C (15.0 g, 10% purity) and TsOH (60.6 g, 352 mmol) under N₂ atmosphere. The suspension is degassed under vacuum and purged with H₂ several times. The mixture is stirred at 25° C., for 3 hrs under H₂ (45 psi) atmosphere. TLC (DCM:MeOH=10:1, PMA) indicated Compound 5A is consumed completely and one new spot (R_{f}=0.04) is formed. The reaction mixture is filtered and concentrated (≤40° C.) under reduced pressure to give a residue. Diluted with anhydrous DCM (500 mL, dried overnight with 4 Å molecular sieves (dried at 300° C., for 12 hrs)) and concentrate to give a residue and run Karl Fisher (KF) to check for water content. This is repeated 3 times with anhydrous DCM (500 mL) dilutions and concentration to give NAcegal-Linker-TMSOTf (205 g, 95.8% yield, TsOH salt) as a foamy white solid, ¹H NMR: δ 7.91 (d, J=9.03 Hz, 1H), 7.53-7.86 (m, 2H), 7.49 (d, J=8.03 Hz, 2H), 7.13 (d, J=8.03 Hz, 2H), 5.22 (d, J=3.26 Hz, 1H), 4.98 (dd, J=11.29, 3.26 Hz, 1H), 4.57 (d, J=8.53 Hz, 1H), 3.99-4.05 (m, 3H), 3.87-3.94 (m, 1H), 3.79-3.85 (m, 1H), 3.51-3.62 (m, 5H), 2.96 (br t, J=5.14 Hz, 2H), 2.29 (s, 3H), 2.10 (s, 3H), 2.00 (s, 3H), 1.89 (s, 3H), 1.78 (s, 3H).

### Scheme for the Preparation of TRIS-PEG2-CBZ

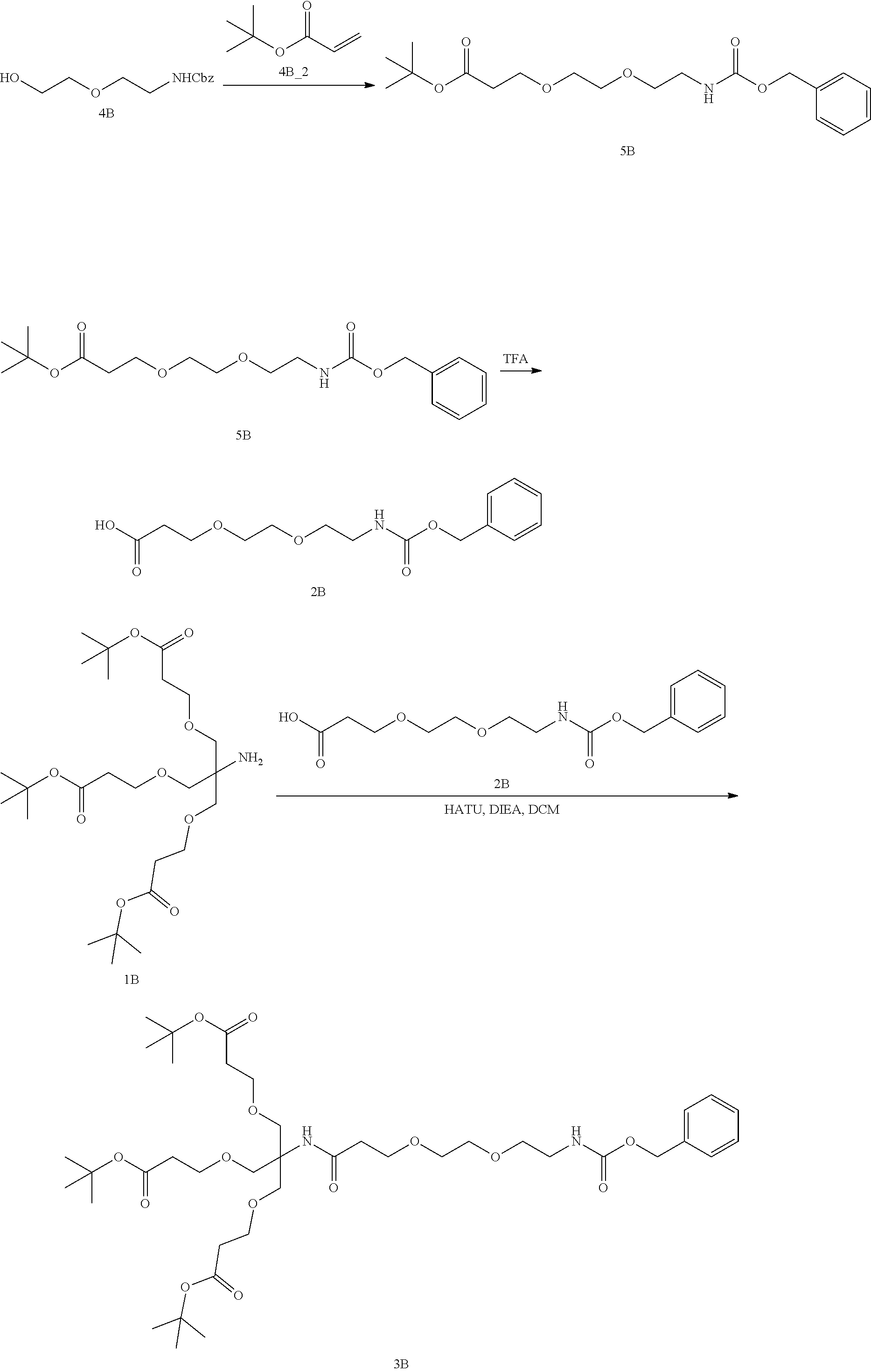

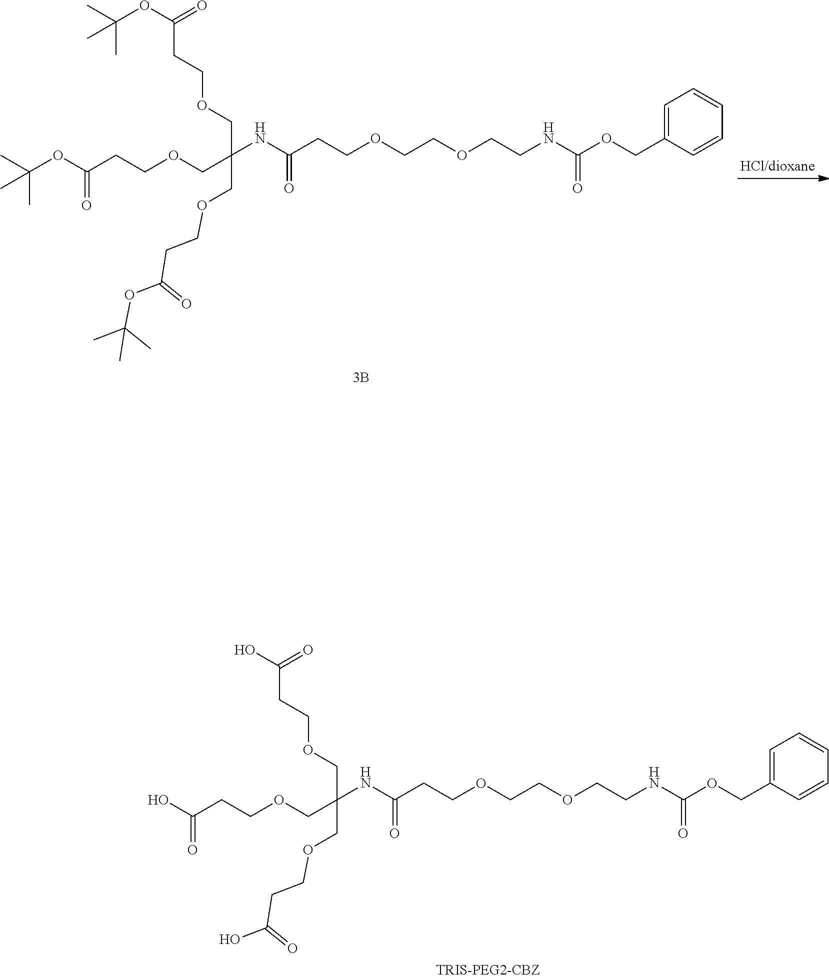 [SMILES: CC(C)(C)OC(=O)CCOCC(COCCC(=O)OC(C)(C)C)(COCCC(=O)OC(C)(C)C)NC(=O)CCOCCOCCNC(=O)OCc1ccccc1 ; O=C(O)CCOCC(COCCC(=O)O)(COCCC(=O)O)NC(=O)CCOCCOCCNC(=O)OCc1ccccc1]

### General Procedure for Preparation of Compound 5B

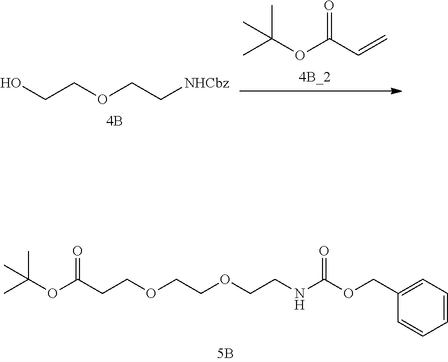

To a solution of Compound 4B (400 g, 1.67 mol, 1.00 eq) and NaOH (10 M, 16.7 mL, 0.10 eq) in THF (2.00 L) is added Compound 4B_2 (1.07 kg, 8.36 mol, 1.20 L, 5.00 eq), the mixture is stirred at 30° C., for 2 hrs. LCMS showed the desired MS is given. Five batches of solution are combined to one batch, then the mixture is diluted with water (6.00 L), extracted with ethyl acetate (3.00 L*3), the combined organic layer is washed with brine (3.00 L), dried over Na₂SO4, filtered and concentrated under vacuum. The crude is purified by column chromatography (SiO₂, petroleum ether:ethyl acetate=100:1-10:1, R_{f}=0.5) to give Compound 5B (2.36 kg, 6.43 mol, 76.9% yield) as light yellow oil. HNMR: δ 7.31-7.36 (m, 5H), 5.38 (s, 1H), 5.11-5.16 (m, 2H), 3.75 (t, J=6.4 Hz), 3.54-3.62 (m, 6H), 3.39 (d, J=5.2 Hz), 2.61 (t, J=6.0 Hz).

General Procedure for Preparation of 3-oxo-1-phenyl-2,7,10-trioxa-4-azatridecan-13-oic Acid (Compound 2B Below)

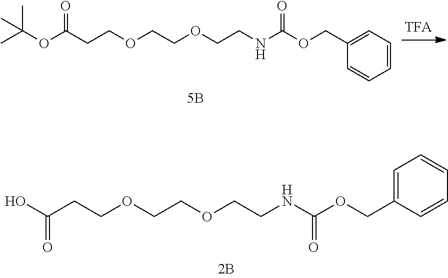 [SMILES: CC(C)(C)OC(=O)CCOCCOCCNC(=O)OCc1ccccc1 ; O=C(O)CCOCCOCCNC(=O)OCc1ccccc1]

To a solution of Compound 5B (741 g, 2.02 mol, 1.00 eq) in DCM (2.80 L) is added TFA (1.43 kg, 12.5 mol, 928 mL, 6.22 eq), the mixture is stirred at 25° C., for 3 hrs. LCMS showed the desired MS is given. The mixture is diluted with DCM (5.00 L), washed with water (3.00 L*3), brine (2.00 L), the combined organic layer is dried over Na₂SO4, filtered and concentrated under vacuum to give Compound 2B (1800 g, crude) as light yellow oil. HNMR: δ 9.46 (s, 5H), 7.27-7.34 (m, 5H), 6.50-6.65 (m, 1H), 5.71 (s, 1H), 5.10-5.15 (m, 2H), 3.68-3.70 (m, 14H), 3.58-3.61 (m, 6H), 3.39 (s, 2H), 2.55 (s, 6H), 2.44 (s, 2H).

### General Procedure for Preparation of Compound 3B

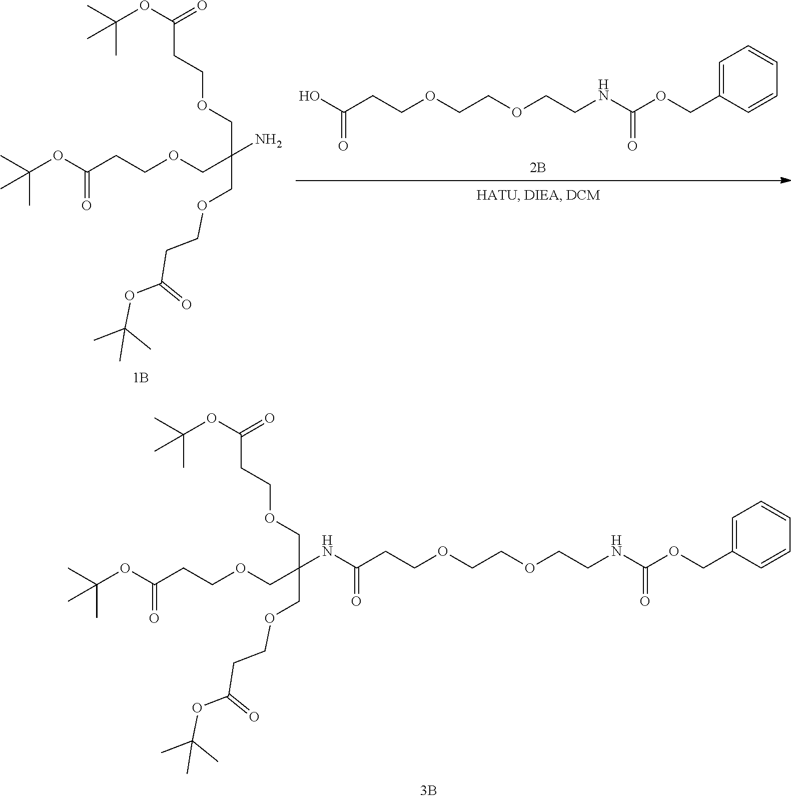 [SMILES: CC(C)(C)OC(=O)CCOCC(N)(COCCC(=O)OC(C)(C)C)COCCC(=O)OC(C)(C)C ; O=C(O)CCOCCOCCNC(=O)OCc1ccccc1 ; CC(C)(C)OC(=O)CCOCC(COCCC(=O)OC(C)(C)C)(COCCC(=O)OC(C)(C)C)NC(=O)CCOCCOCCNC(=O)OCc1ccccc1]

To a solution of Compound 2B (375 g, 999 mmol, 83.0% purity, 1.00 eq) in DCM (1.80 L) is added HATU (570 g, 1.50 mol, 1.50 eq) and DIEA (258 g, 2.00 mol, 348 mL, 2.00 eq) at 0° C., the mixture is stirred at 0° C., for 30 min, then Compound 1B (606 g, 1.20 mol, 1.20 eq) is added, the mixture is stirred at 25° C., for 1 hr. LCMS showed desired MS is given. The mixture is combined to one batch, then the mixture is diluted with DCM (5.00 L), washed with 1 N HCl aqueous solution (2.00 L*2), then the organic layer is washed with saturated Na₂CO₃ aqueous solution (2.00 L*2) and brine (2.00 L), the organic layer is dried over Na₂SO4, filtered and concentrated under vacuum to give Compound 3B (3.88 kg, crude) as yellow oil.

### General Procedure for Preparation of TRIS-PEG2-CBZ.

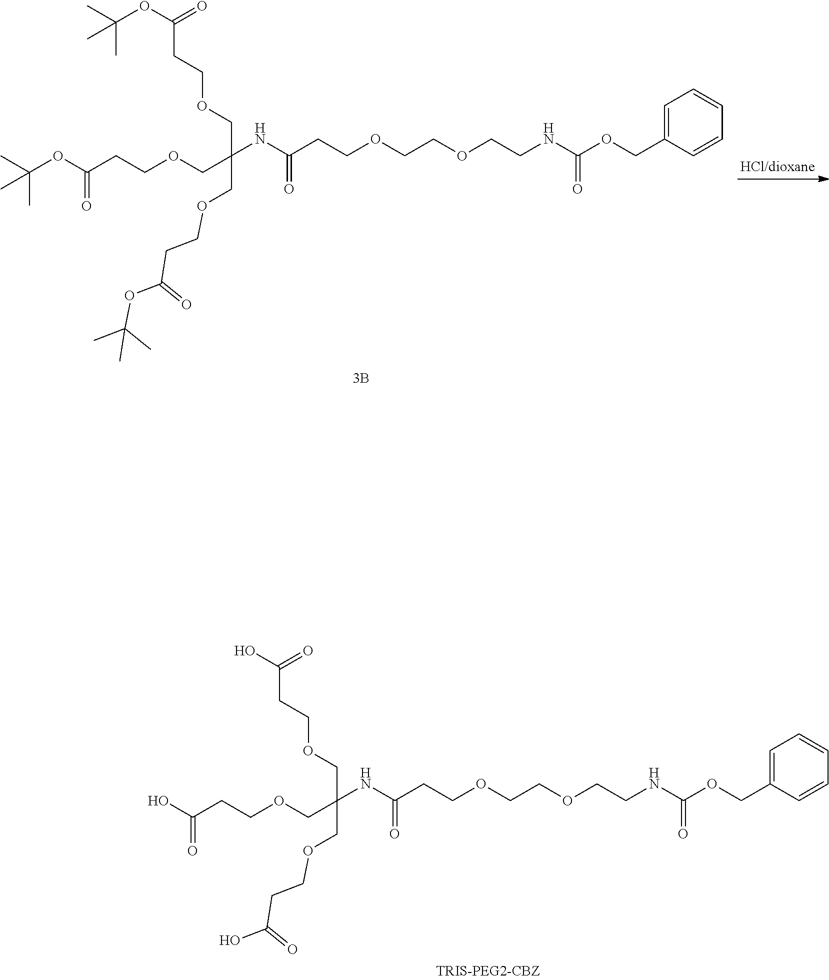 [SMILES: CC(C)(C)OC(=O)CCOCC(COCCC(=O)OC(C)(C)C)(COCCC(=O)OC(C)(C)C)NC(=O)CCOCCOCCNC(=O)OCc1ccccc1 ; O=C(O)CCOCC(COCCC(=O)O)(COCCC(=O)O)NC(=O)CCOCCOCCNC(=O)OCc1ccccc1]

A solution of Compound 3B (775 g, 487 mmol, 50.3% purity, 1.00 eq) in HCl/dioxane (4 M, 2.91 L, 23.8 eq) is stirred at 25° C., for 2 hrs. LCMS showed the desired MS is given. The mixture is concentrated under vacuum to give a residue. Then the combined residue is diluted with DCM (5.00 L), adjusted to pH=8 with 2.5 M NaOH aqueous solution, and separated. The aqueous phase is extracted with DCM (3.00 L) again, then the aqueous solution is adjusted to pH=3 with 1 N HCl aqueous solution, then extracted with DCM (5.00 L*2), the combined organic layer is washed with brine (3.00 L), dried over Na₂SO4, filtered and concentrated under vacuum. The crude is purified by column chromatography (SiO₂, DCM:MeOH=0:1-12:1, 0.1% HOAc, R_{f}=0.4). The residue is diluted with DCM (5.00 L), adjusted to pH=8 with 2.5 M NaOH aqueous solution, separated, the aqueous solution is extracted with DCM (3.00 L) again, then the aqueous solution is adjusted to pH=3 with 6 N HCl aqueous solution, extracted with DCM:MeOH=10:1 (5.00 L*2), the combined organic layer is washed with brine (2.00 L), dried over Na₂SO4, filtered and concentrated under vacuum to give a residue. Then the residue is diluted with MeCN (5.00 L), concentrated under vacuum, repeat this procedure twice to remove water to give TRIS-PEG2-CBZ (1.25 kg, 1.91 mol, 78.1% yield, 95.8% purity) as light yellow oil. ¹HNMR: 400 MHZ, MeOD, δ 7.30-7.35 (5H), 5.07 (s, 2H), 3.65-3.70 (m, 16H), 3.59 (s, 4H), 3.45 (t, J=5.6 Hz), 2.51 (t, J=6.0 Hz), 2.43 (t, 6.4 Hz).

### Scheme for the Preparation of TriNGal-TRIS-Peg2-Phosph 8c

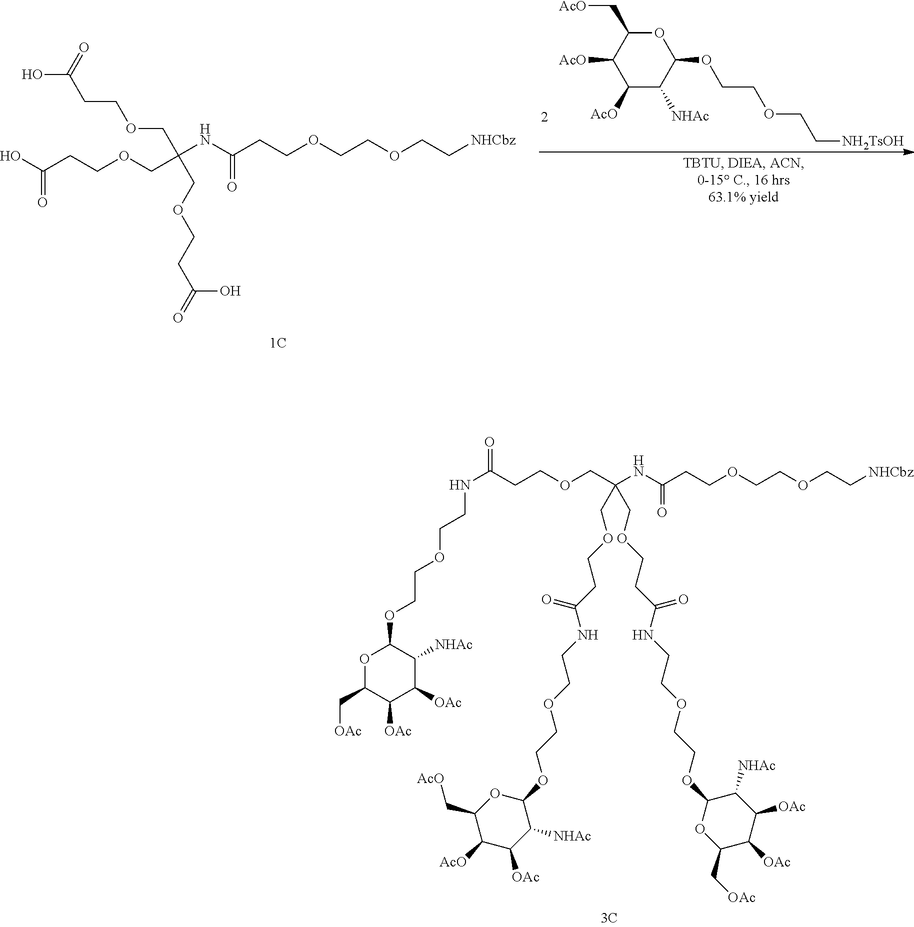

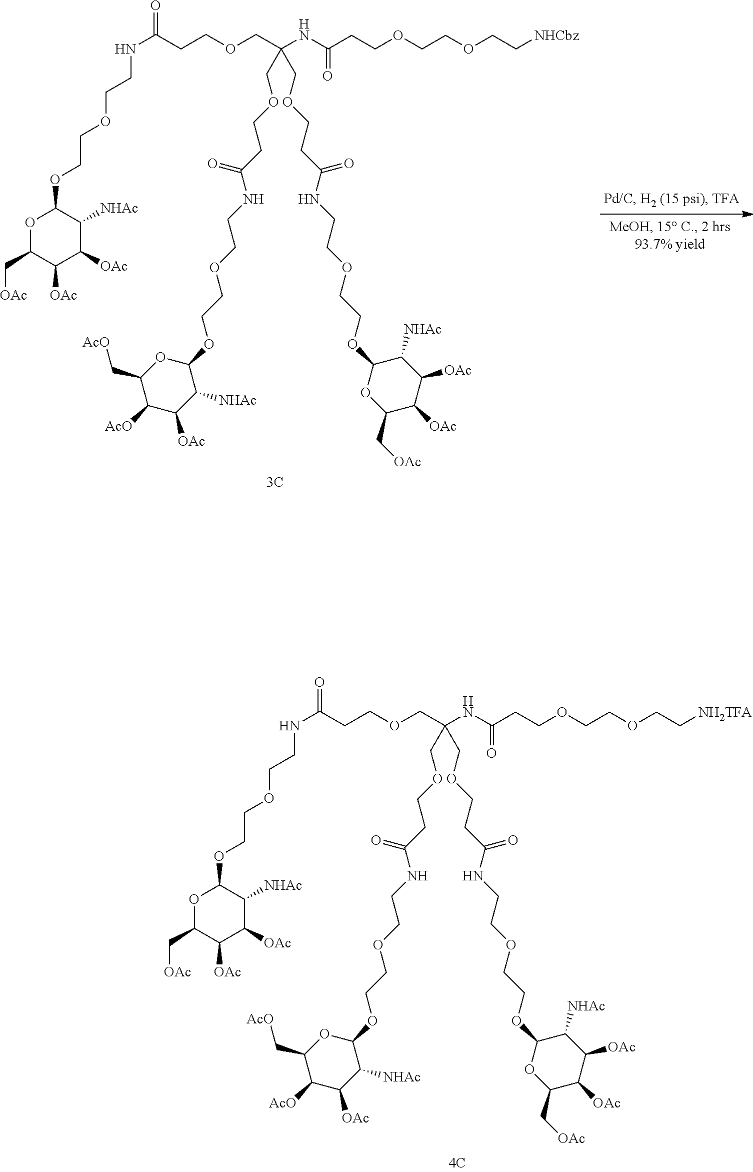

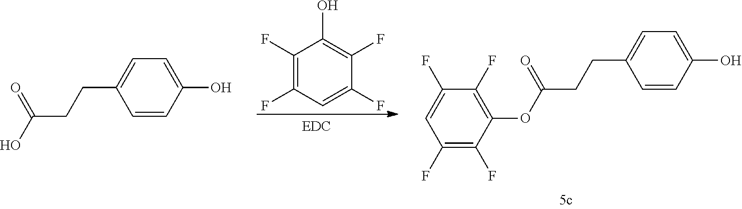 [SMILES: O=C(O)CCc1ccc(O)cc1 ; Oc1c(F)c(F)cc(F)c1F ; O=C(CCc1ccc(O)cc1)Oc1c(F)c(F)cc(F)c1F]

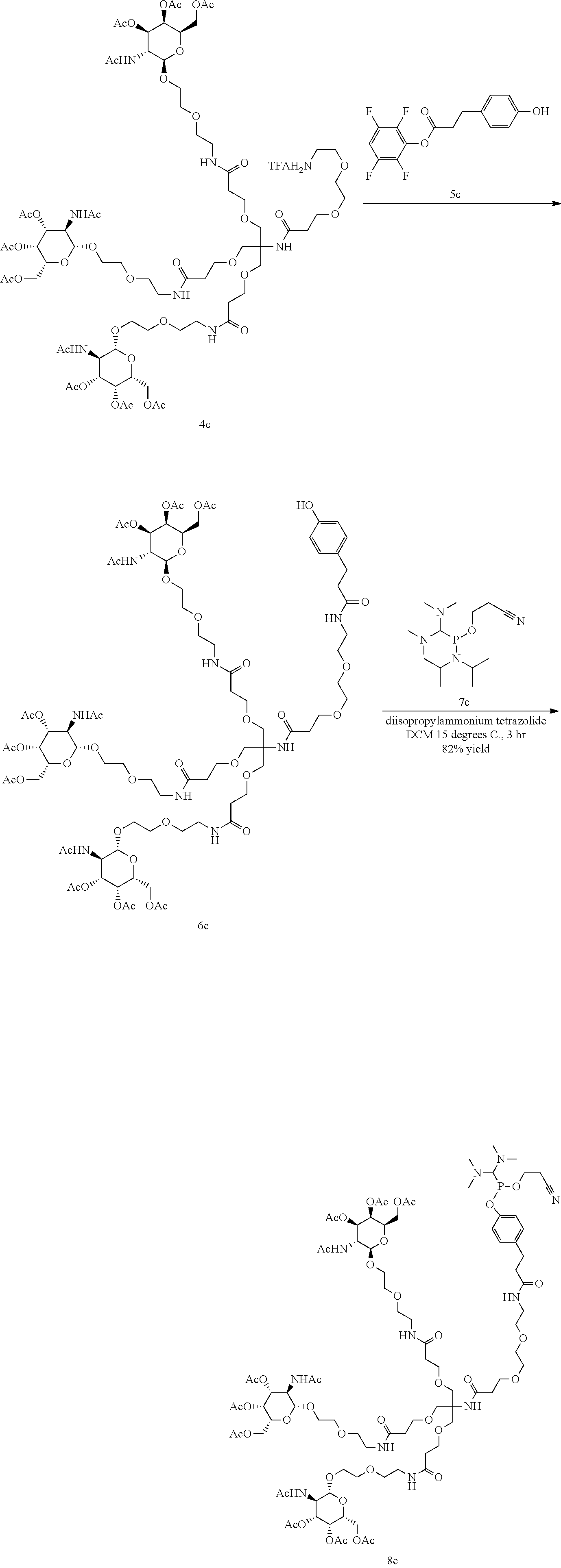

### TriGNal-TRIS-Peg2-Phosph 8c

### General Procedure for Preparation of Compound 3C

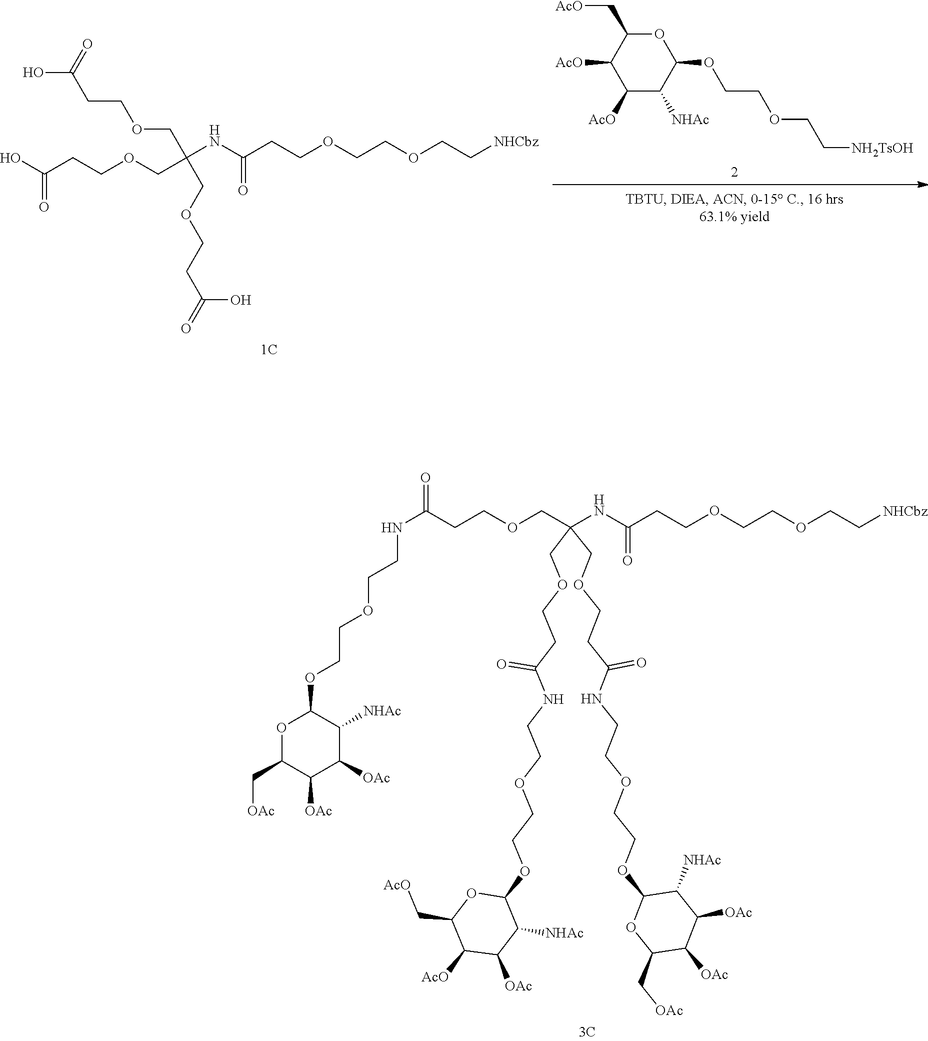

To a solution of Compound 1C (155 g, 245 mmol, 1.00 eq) in ACN (1500 mL) is added TBTU (260 g, 811 mmol, 3.30 eq), DIEA (209 g, 1.62 mol, 282 mL, 6.60 eq) and Compound 2C (492 g, 811 mmol, 3.30 eq, TsOH) at 0° C., the mixture is stirred at 15° C., for 16 hrs. LCMS showed the desired MS is given. The mixture is concentrated under vacuum to give a residue, then the mixture is diluted with DCM (2000 mL), washed with 1 N HCl aqueous solution (700 mL*2), then saturated NaHCO₃ aqueous solution (700 mL*2) and concentrated under vacuum. The crude is purified by column chromatography to give Compound 3C (304 g, 155 mmol, 63.1% yield, 96.0% purity) as a yellow solid.

### General Procedure for Preparation of Compound 4C

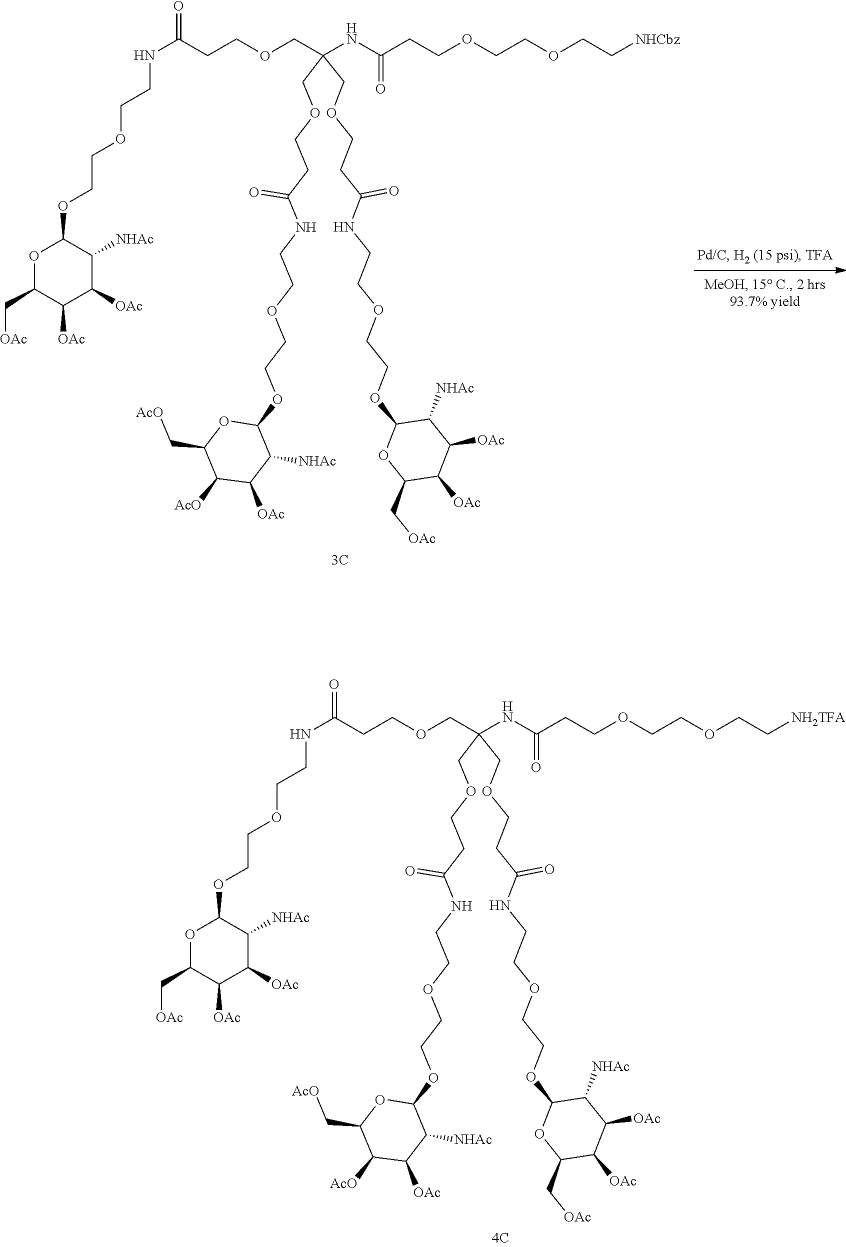

Two batches solution of Compound 3C (55.0 g, 29.2 mmol, 1.00 eq) in MeOH (1600 mL) is added Pd/C (6.60 g, 19.1 mmol, 10.0% purity) and TFA (3.34 g, 29.2 mmol, 2.17 mL, 1.00 eq), the mixture is degassed under vacuum and purged with H₂. The mixture is stirred under H₂ (15 psi) at 15° C., for 2 hours. LCMS showed the desired MS is given. The mixture is filtered and the filtrate is concentrated under vacuum to give Compound 4C (106 g, 54.8 mmol, 93.7% yield, 96.2% purity, TFA) as a white solid.

### General Procedure for Preparation of Compound 5C

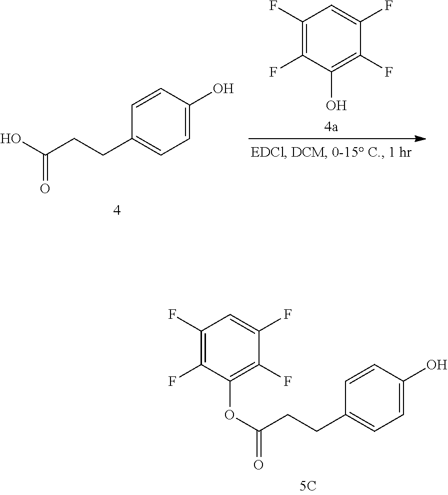 [SMILES: O=C(O)CCc1ccc(O)cc1 ; Oc1c(F)c(F)cc(F)c1F ; O=C(CCc1ccc(O)cc1)Oc1c(F)c(F)cc(F)c1F]

Two batches in parallel. To a solution of EDCI (28.8 g, 150 mmol, 1.00 eq) in DCM (125 mL) is added compound 4a (25.0 g, 150 mmol, 1.00 eq) dropwise at 0° C., then the mixture is added to compound 4 (25.0 g, 150 mmol, 1.00 eq) in DCM (125 mL) at 0° C., then the mixture is stirred at 25° C., for 1 hr. TLC (Petroleum ether:Ethyl acetate=3:1, R_{f}=0.45) showed the reactant is consumed and one new spot is formed. The reaction mixture is diluted with DCM (100 mL) then washed with aq. NaHCO₃ (250 mL*1) and brine (250 mL), dried over Na₂SO4, filtered and concentrated under reduced pressure to give a residue. The residue is purified by column chromatography (SiO₂, Petroleum ether:Ethyl acetate=100:1 to 3:1), TLC (SiO₂, Petroleum ether:Ethyl acetate=3:1), R_{f}=0.45, then concentrated under reduced pressure to give a residue. Compound 5C (57.0 g, 176 mmol, 58.4% yield, 96.9% purity) is obtained as colorless oil and confirmed ¹HNMR: EW33072-2-P1A, 400 MHZ, DMSO δ 9.21 (s, 1H), 7.07-7.09 (m, 2H), 6.67-6.70 (m, 2H), 3.02-3.04 (m, 2H), 2.86-2.90 (m, 2H)

### General Procedure for Preparation of Compound 6

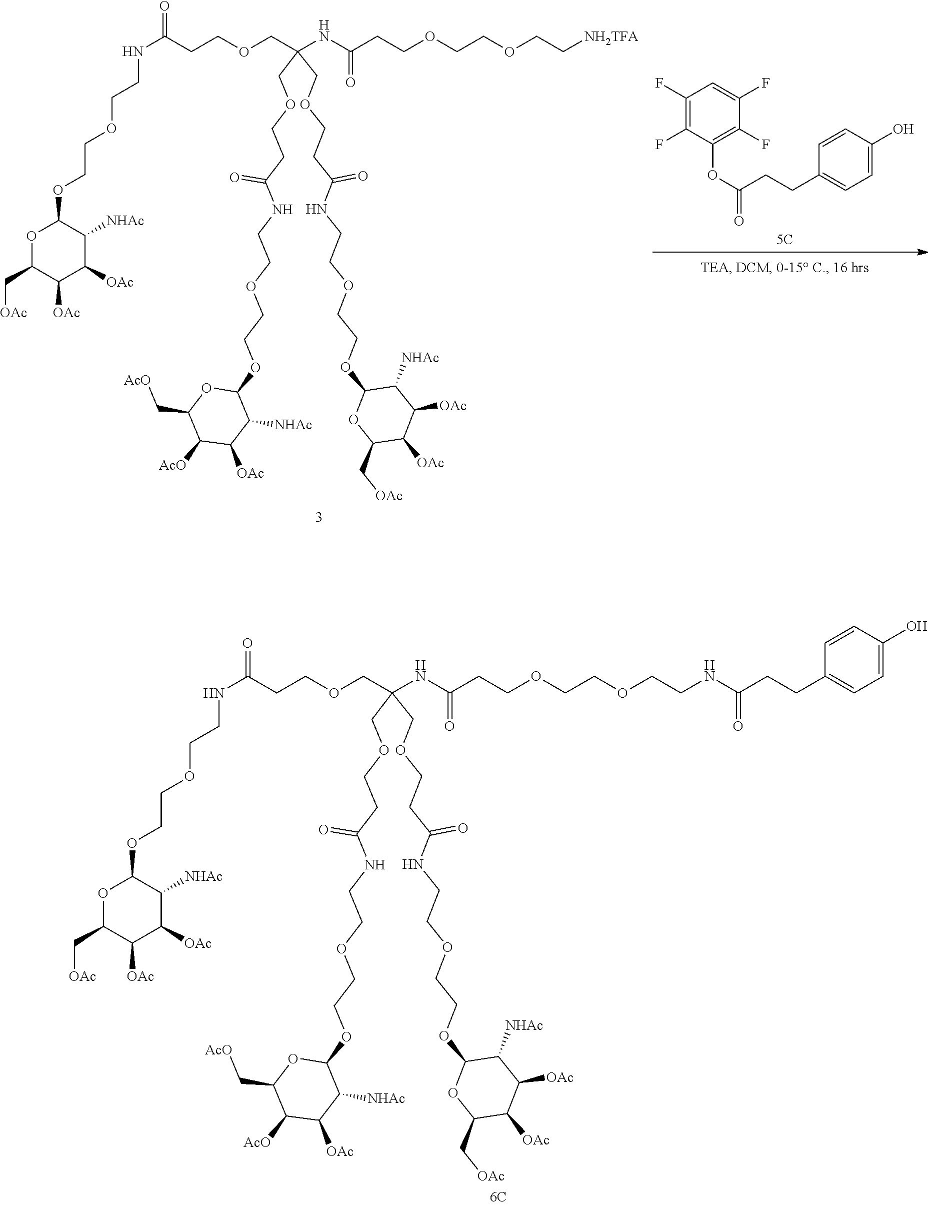

To a mixture of compound 3 (79.0 g, 41.0 mmol, 96.4% purity, 1.00 eq, TFA) and compound 6C (14.2 g, 43.8 mmol, 96.9% purity, 1.07 eq) in DCM (800 mL) is added TEA (16.6 g, 164 mmol, 22.8 mL, 4.00 eq) dropwise at 0° C., the mixture is stirred at 15° C., for 16 hrs. LCMS (EW33072-12-P1B, Rt=0.844 min) showed the desired mass is detected. The reaction mixture is diluted with DCM (400 mL) and washed with aq. NaHCO₃ (400 mL*1) and brine(400 mL*1), then the mixture is diluted with DCM (2.00 L) and washed with 0.7 M Na₂CO₃ (1000 mL*3) and brine(800 mL*3), dried over Na₂SO4, filtered and concentrated under reduced pressure to give a residue. The residue is used to next step directly without purification. Compound 6 (80.0 g, crude) is obtained as white solid and confirmed via ¹HNMR: EW33072-12-P1A, 400 MHz, MeOD & 7.02-7.04 (m, 2H), 6.68-6.70 (m, 2H), 5.34-5.35 (s, 3H), 5.07-5.08 (d, J=4.00 Hz, 3H), 4.62-4.64 (d, J=8.00 Hz, 3H), 3.71-4.16 (m, 16H), 3.31-3.70 (m, 44H), 2.80-2.83 (m, 2H), 2.68 (m, 2H), 2.46-2.47 (m, 10H), 2.14 (s, 9H), 2.03 (s, 9H), 1.94-1.95 (d, J=4.00 Hz, 18H).

### General Procedure for Preparation of TriGNal-TRIS-Peg2-Phosph 8c

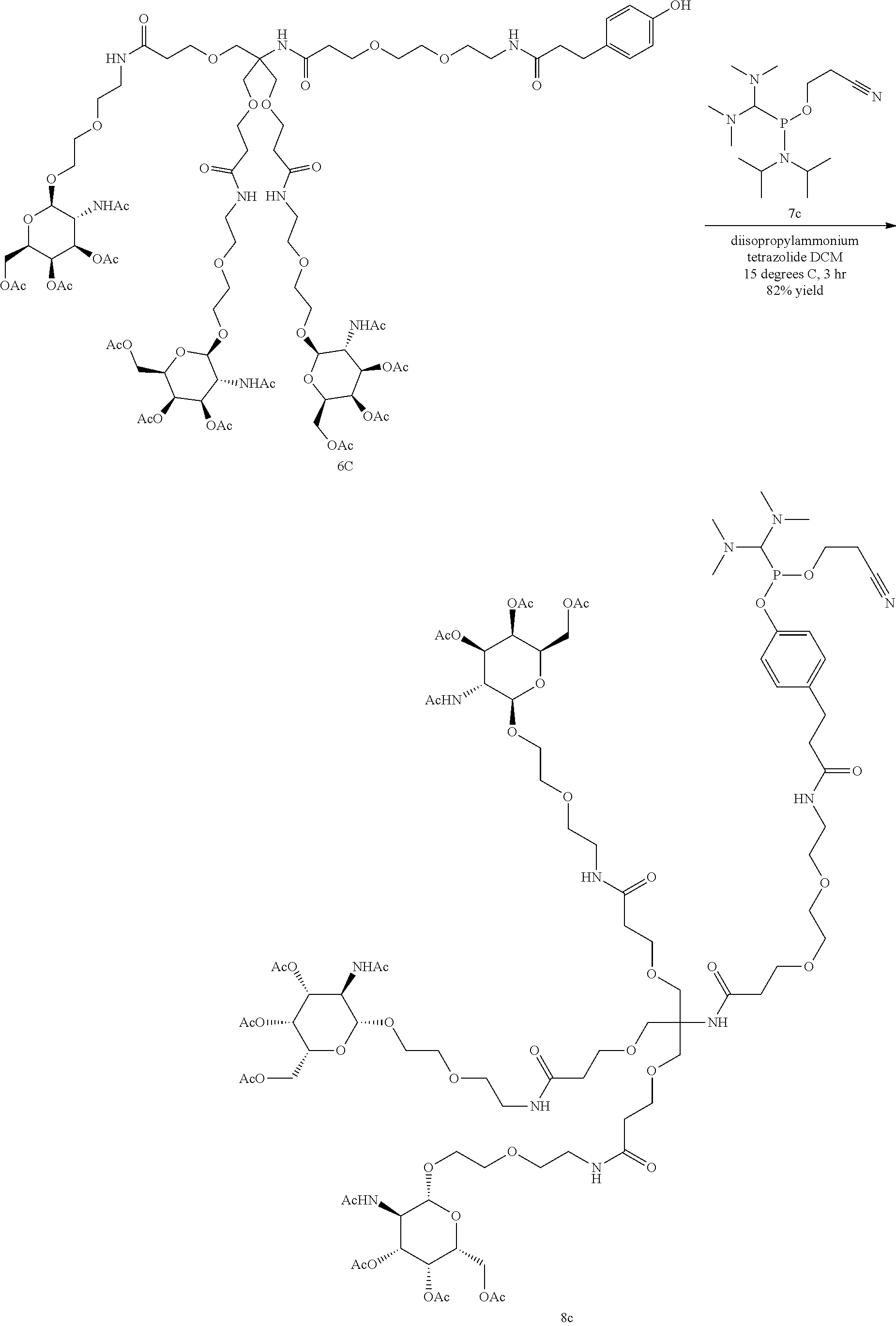

Two batches are synthesized in parallel. To a solution of compound 6C (40.0 g, 21.1 mmol, 1.00 eq in DCM (600 mL) is added diisopropylammonium tetrazolide (3.62 g, 21.1 mmol, 1.00 eq) and compound 7c (6.37 g, 21.1 mmol, 6.71 mL, 1.00 eq) in DCM (8.00 mL) drop-wise, the mixture is stirred at 30° C., for 1 hr, then added compound 7c (3.18 g, 10.6 mmol, 3.35 mL, 0.50 eq) in DCM (8.00 mL) drop-wise, the mixture is stirred at 30° C., for 30 mins, then added compound 7c (3.18 g, 10.6 mmol, 3.35 mL, 0.50 eq) in DCM (8.00 mL) drop-wise, the mixture is stirred at 30° C., for 1.5 hrs. LCMS (EW33072-17-P1C1, Rt=0.921 min) showed the desired MS+1 is detected. LCMS (EW33072-17-P1C₂, Rt=0.919 min) showed the desired MS+1 is detected. Two batches are combined for work-up. The mixture is diluted with DCM (1.20 L), washed with saturated NaHCO₃ aqueous solution (1.60 L*2), 3% DMF in H₂O (1.60 L*2), H₂O (1.60 L*3), brine (1.60 L), dried over Na₂SO4, filtered and concentrated under reduced pressure to give a residue. The residue is purified by column chromatography (SiO₂, DCM:MeOH:TEA=100:3:2) TLC (SiO₂, DCM:MeOH=10:1, R_{f}=0.45), then concentrated under reduced pressure to give a residue. Compound 8C (76.0 g, 34.8 mmol, 82.5% yield, 96.0% purity) is obtained as white solid and confirmed via ¹HNMR: EW33072-19-PIC, 400 MHZ, MeOD δ 7.13-7.15 (d. J=8.50 Hz, 2H), 6.95-6.97 (dd, J=8.38, 1.13 Hz, 2H), 5.34 (d, J=2.88 Hz, 3H), 0.09 (dd, J=11.26, 3.38 Hz, 3H), 4.64 (d, J=8.50 Hz, 3H), 3.99-4.20 (m, 12H), 3.88-3.98 (m, 5H), 3.66-3.83 (m, 20H), 3.51-3.65 (m, 17H), 3.33-3.50 (m, 9H), 2.87 (t, J=7.63 Hz, 2H), 2.76 (t. J=5.94 Hz, 2H), 2.42-2.50 (m, 10H), 2.14 (s, 9H), 2.03 (s, 9H), 1.94-1.95 (d, J=6.13 Hz, 18H), 1.24-1.26 (d. J=6.75 Hz, 6H), 1.18-1.20 (d, J=6.75 Hz, 6H)

### Example 16: Delivery to Central Nervous System Injection

siRNA's with modifications and hydrophobic conjugates as described are injected intracerebroventricularly or intrathecally according to published procedures (Alterman, J. F., Godinho, B. M. D. C., Hassler, M. R. et al. A divalent siRNA chemical scaffold for potent and sustained modulation of gene expression throughout the central nervous system. Nat Biotechnol 37, 884-894 (2019). https://doi.org/10.1038/s41587-019-0205-0, Njoo, C., Heinl, C., Kuner, R. In Vivo SiRNA Transfection and Gene Knockdown in Spinal Cord via Rapid Noninvasive Lumbar Intrathecal Injections in Mice. J. Vis. Exp. (85), e51229, doi: 10.3791/51229 (2014)), 14 days post injection, mice are euthanized, brain hemispheres are harvested, frozen, later homogenized, and tested for PLIN1 mRNA and protein expression.

### Example 17: Bullet Point Description of a Modified siRNA

An example PLIN1-targeting siRNA is as follows:
21 mer:
   a. 19 base pairs
   b. 2 nt overhangs
antisense strand:
   a. vinyl phosphonate (VP) at 5′ end
   b. 2 phosphorothioate bonds (PS) at each end
sense strand:
   a. hydrophobic group (C16-C18) at 5′ end
   b. optional 0-2 PS at 5′ end
   c. 2 PS at 3′ end
modification pattern of 2′ fluoro and 2′ methyl groups.

### Example 18: Modification Motif 1

An example PLIN1-targeting siRNA includes a combination of the following modifications:
Position 9 (from 5′ to 3′) of the sense strand is 2′ F.
If position 9 is a pyrimidine then all purines in the Sense Strand are 2′OMe, and 1-5 pyrimidines between positions 5 and 11 are 2′ F provided that there are never three 2′F modifications in a row.
If position 9 is a purine then all pyrimidines in the Sense Strand are 2′OMe, and 1-5 purines between positions 5 and 11 are 2′ F provided that there are never three 2′F modifications in a row.
Antisense strand odd-numbered positions are 2′OMe and even-numbered positions are a mixture of 2′ F, 2′OMe and 2′ deoxy.

### Example 19: Modification Motif 2

An example PLIN1-targeting siRNA includes a combination of the following modifications:
Position 9 (from 5′ to 3′) of the sense strand is 2′ deoxy.
Sense strand positions 5, 7 and 8 are 2′ F.
All pyrimidines in positions 10-21 are 2′ OMe, and purines are a mixture of 2′ OMe and 2′ F. Alternatively, all purines in positions 10-21 are 2′ OMe and all pyrimidines in positions 10-21 are a mixture of 2′ OMe and 2′ F.
Antisense strand odd-numbered positions are 2′OMe and even-numbered positions are a mixture of 2′ F, 2′OMe and 2′ deoxy.

### Example 20: Example siRNA Sequences

The base sequences of some example siRNAs are shown in Table 22.

TABLE 22
Base sequences of example siRNAs

| | Sense | Sense Strand | Antisense | Antisense Strand |
|---|---|---|---|---|
| siRNA | SEQ ID | Base Sequence | SEQ ID | Base Sequence |
| Name | NO: | (5′ to 3′) | NO: | (5′ to 3′) |
| ETD01510 | 5970 | CAACACUCUUUCUCGACAAUU | 5990 | UUGUCGAGAAAGAGUGUUGUU |
| ETD01540 | 5971 | CAACACUCUUUCUCGACAAUU | 5991 | UUGUCGAGAAAGAGUGUUGUU |
| ETD01624 | 5971 | CAACACUCUUUCUCGACAAUU | 5992 | 5VPUUGUCGAGAAAGAGUGUUGUU |
| ETD01751 | 5971 | CAACACUCUUUCUCGACAAUU | 5993 | 5VPUUGUCGAGAAAGAGUGUUGUU |
| ETD01754 | 5972 | UAAGAGUAAUUGCCUAACAUU | 5994 | UGUUAGGCAAUUACUCUUAUU |
| ETD01755 | 5973 | GUAAUUGCCUAACUUGAUAUU | 5995 | UAUCAAGUUAGGCAAUUACUU |
| ETD01756 | 5974 | UUUGACACAUUCUUAGCAAUU | 5996 | UUGCUAAGAAUGUGUCAAAUU |
| ETD01899 | 5975 | CAGUUUUUAAGGGACACCAUU | 5997 | UGGUGUCCCUUAAAAACUGUU |
| ETD01900 | 5976 | UUUUUAAGGGACACCAGAAUU | 5998 | UUCUGGUGUCCCUUAAAAAUU |
| ETD01901 | 5977 | UUUUGACACAUUCUUAGCAUU | 5999 | UGCUAAGAAUGUGUCAAAAUU |
| ETD01902 | 5978 | UUGACACAUUCUUAGCACAUU | 6000 | UGUGCUAAGAAUGUGUCAAUU |
| ETD01903 | 5979 | ACAUUCUUAGCACUGAACAUU | 6001 | UGUUCAGUGCUAAGAAUGUUU |
| ETD01904 | 5980 | UGCAUAGUCACUCUUUUGAUU | 6002 | UCAAAAGAGUGACUAUGCAUU |
| ETD01905 | 5981 | AACUACUGCAUAAUAUGGAUU | 6003 | UCCAUAUUAUGCAGUAGUUUU |
| ETD01510 | 5982 | CAACACUCUUUCUCGACAA | 6004 | UUGUCGAGAAAGAGUGUUG |
| ETD01540 | 5983 | CAACACUCUUUCUCGACAA | 6005 | UUGUCGAGAAAGAGUGUUG |
| ETD01624 | 5983 | CAACACUCUUUCUCGACAA | 6006 | 5VPUUGUCGAGAAAGAGUGUUG |
| ETD01751 | 5983 | CAACACUCUUUCUCGACAA | 6007 | 5VPUUGUCGAGAAAGAGUGUUG |
| ETD01754 | 5984 | UAAGAGUAAUUGCCUAACA | 6008 | UGUUAGGCAAUUACUCUUA |
| ETD01755 | 5985 | GUAAUUGCCUAACUUGAUA | 6009 | UAUCAAGUUAGGCAAUUAC |
| ETD01756 | 5986 | UUUGACACAUUCUUAGCAA | 6010 | UUGCUAAGAAUGUGUCAAA |
| ETD01899 | 1908 | CAGUUUUUAAGGGACACCA | 4806 | UGGUGUCCCUUAAAAACUG |
| ETD01900 | 5987 | UUUUUAAGGGACACCAGAA | 6011 | UUCUGGUGUCCCUUAAAAA |
| ETD01901 | 2124 | UUUUGACACAUUCUUAGCA | 5022 | UGCUAAGAAUGUGUCAAAA |
| ETD01902 | 5988 | UUGACACAUUCUUAGCACA | 6012 | UGUGCUAAGAAUGUGUCAA |
| ETD01903 | 5989 | ACAUUCUUAGCACUGAACA | 6013 | UGUUCAGUGCUAAGAAUGU |
| ETD01904 | 2390 | UGCAUAGUCACUCUUUUGA | 5288 | UCAAAAGAGUGACUAUGCA |
| ETD01905 | 2787 | AACUACUGCAUAAUAUGGA | 5685 | UCCAUAUUAUGCAGUAGUU |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and compositions within the scope of these claims and their equivalents be covered thereby.

### IV. Sequence Information

Some embodiments include one or more nucleic acid sequences in the following tables:

Sequence Information

| SEQ ID NO: | Description |
|---|---|
| 1-2898 | PLIN1 siRNA sense strand sequences |
| 2899-5796 | PLIN1 siRNA antisense strand sequences |
| 5797-5845 | Modified PLIN1 siRNA sense strand sequences |
| 5846-5894 | Modified PLIN1 siRNA antisense strand sequences |
| 5895-5943 | Alternatively modified PLIN1 siRNA sense strand |
| | sequences |
| 5944-5959 | Examples of modified PLIN1 siRNA sense strand |
| | sequences |
| 5960-5969 | Examples of modified PLIN1 siRNA antisense strand |
| | sequences |
| 5970-5989 | Examples of PLIN1 siRNA sense strand base sequences |
| 5990-6013 | Examples of PLIN1 siRNA antisense strand base |
| | sequences |
| 6014 | Full-length human PLIN1 mRNA sequence (Ensembl Acc. |
| | No. ENST00000300055.10) (human RNA) |
| 6015-6064 | Modification pattern 1S to 50S |
| 6065-6074 | Modification pattern 1AS to 10AS |
| 6075 | Modification pattern ASO1 |

Sequences

| | SEQ | | SEQ | |
|---|---|---|---|---|
| | ID | Sense strand | ID | Antisense strand |
| siRNA Name | NO: | sequence (5′-3′) | NO: | sequence (5′-3′) |
| siRNA 1 | 1 | ACUCUGCAGCCUGGGCUCU | 2899 | AGAGCCCAGGCUGCAGAGU |
| siRNA 2 | 2 | CUCUGCAGCCUGGGCUCUG | 2900 | CAGAGCCCAGGCUGCAGAG |
| siRNA 3 | 3 | UCUGCAGCCUGGGCUCUGU | 2901 | ACAGAGCCCAGGCUGCAGA |
| siRNA 4 | 4 | CUGCAGCCUGGGCUCUGUG | 2902 | CACAGAGCCCAGGCUGCAG |
| siRNA 5 | 5 | UGCAGCCUGGGCUCUGUGA | 2903 | UCACAGAGCCCAGGCUGCA |
| siRNA 6 | 6 | GCAGCCUGGGCUCUGUGAG | 2904 | CUCACAGAGCCCAGGCUGC |
| siRNA 7 | 7 | CAGCCUGGGCUCUGUGAGA | 2905 | UCUCACAGAGCCCAGGCUG |
| siRNA 8 | 8 | AGCCUGGGCUCUGUGAGAC | 2906 | GUCUCACAGAGCCCAGGCU |
| siRNA 9 | 9 | GCCUGGGCUCUGUGAGACU | 2907 | AGUCUCACAGAGCCCAGGC |
| siRNA 10 | 10 | CCUGGGCUCUGUGAGACUG | 2908 | CAGUCUCACAGAGCCCAGG |
| siRNA 11 | 11 | CUGGGCUCUGUGAGACUGA | 2909 | UCAGUCUCACAGAGCCCAG |
| siRNA 12 | 12 | UGGGCUCUGUGAGACUGAG | 2910 | CUCAGUCUCACAGAGCCCA |
| siRNA 13 | 13 | GGGCUCUGUGAGACUGAGG | 2911 | CCUCAGUCUCACAGAGCCC |
| siRNA 14 | 14 | GGCUCUGUGAGACUGAGGU | 2912 | ACCUCAGUCUCACAGAGCC |
| siRNA 15 | 15 | GCUCUGUGAGACUGAGGUG | 2913 | CACCUCAGUCUCACAGAGC |
| siRNA 16 | 16 | CUCUGUGAGACUGAGGUGG | 2914 | CCACCUCAGUCUCACAGAG |
| siRNA 17 | 17 | UCUGUGAGACUGAGGUGGC | 2915 | GCCACCUCAGUCUCACAGA |
| siRNA 18 | 18 | CUGUGAGACUGAGGUGGCG | 2916 | CGCCACCUCAGUCUCACAG |
| siRNA 19 | 19 | UGUGAGACUGAGGUGGCGG | 2917 | CCGCCACCUCAGUCUCACA |
| siRNA 20 | 20 | GUGAGACUGAGGUGGCGGU | 2918 | ACCGCCACCUCAGUCUCAC |
| siRNA 21 | 21 | UGAGACUGAGGUGGCGGUC | 2919 | GACCGCCACCUCAGUCUCA |
| siRNA 22 | 22 | GAGACUGAGGUGGCGGUCA | 2920 | UGACCGCCACCUCAGUCUC |
| siRNA 23 | 23 | AGACUGAGGUGGCGGUCAG | 2921 | CUGACCGCCACCUCAGUCU |
| siRNA 24 | 24 | GACUGAGGUGGCGGUCAGC | 2922 | GCUGACCGCCACCUCAGUC |
| siRNA 25 | 25 | ACUGAGGUGGCGGUCAGCC | 2923 | GGCUGACCGCCACCUCAGU |
| siRNA 26 | 26 | CUGAGGUGGCGGUCAGCCG | 2924 | CGGCUGACCGCCACCUCAG |
| siRNA 27 | 27 | UGAGGUGGCGGUCAGCCGG | 2925 | CCGGCUGACCGCCACCUCA |
| siRNA 28 | 28 | GAGGUGGCGGUCAGCCGGA | 2926 | UCCGGCUGACCGCCACCUC |
| siRNA 29 | 29 | AGGUGGCGGUCAGCCGGAG | 2927 | CUCCGGCUGACCGCCACCU |
| siRNA 30 | 30 | GGUGGCGGUCAGCCGGAGU | 2928 | ACUCCGGCUGACCGCCACC |
| siRNA 31 | 31 | GUGGCGGUCAGCCGGAGUG | 2929 | CACUCCGGCUGACCGCCAC |
| siRNA 32 | 32 | UGGCGGUCAGCCGGAGUGA | 2930 | UCACUCCGGCUGACCGCCA |
| siRNA 33 | 33 | GGCGGUCAGCCGGAGUGAG | 2931 | CUCACUCCGGCUGACCGCC |
| siRNA 34 | 34 | GCGGUCAGCCGGAGUGAGU | 2932 | ACUCACUCCGGCUGACCGC |
| siRNA 35 | 35 | CGGUCAGCCGGAGUGAGUG | 2933 | CACUCACUCCGGCUGACCG |
| siRNA 36 | 36 | GGUCAGCCGGAGUGAGUGU | 2934 | ACACUCACUCCGGCUGACC |
| siRNA 37 | 37 | GUCAGCCGGAGUGAGUGUU | 2935 | AACACUCACUCCGGCUGAC |
| siRNA 38 | 38 | UCAGCCGGAGUGAGUGUUG | 2936 | CAACACUCACUCCGGCUGA |
| siRNA 39 | 39 | CAGCCGGAGUGAGUGUUGG | 2937 | CCAACACUCACUCCGGCUG |
| siRNA 40 | 40 | AGCCGGAGUGAGUGUUGGG | 2938 | CCCAACACUCACUCCGGCU |
| siRNA 41 | 41 | GCCGGAGUGAGUGUUGGGG | 2939 | CCCCAACACUCACUCCGGC |
| siRNA 42 | 42 | CCGGAGUGAGUGUUGGGGU | 2940 | ACCCCAACACUCACUCCGG |
| siRNA 43 | 43 | CGGAGUGAGUGUUGGGGUC | 2941 | GACCCCAACACUCACUCCG |
| siRNA 44 | 44 | GGAGUGAGUGUUGGGGUCC | 2942 | GGACCCCAACACUCACUCC |
| siRNA 45 | 45 | GAGUGAGUGUUGGGGUCCU | 2943 | AGGACCCCAACACUCACUC |
| siRNA 46 | 46 | AGUGAGUGUUGGGGUCCUG | 2944 | CAGGACCCCAACACUCACU |
| siRNA 47 | 47 | GUGAGUGUUGGGGUCCUGG | 2945 | CCAGGACCCCAACACUCAC |
| siRNA 48 | 48 | UGAGUGUUGGGGUCCUGGG | 2946 | CCCAGGACCCCAACACUCA |
| siRNA 49 | 49 | GAGUGUUGGGGUCCUGGGG | 2947 | CCCCAGGACCCCAACACUC |
| SIRNA 50 | 50 | AGUGUUGGGGUCCUGGGGC | 2948 | GCCCCAGGACCCCAACACU |
| siRNA 51 | 51 | GUGUUGGGGUCCUGGGGCA | 2949 | UGCCCCAGGACCCCAACAC |
| siRNA 52 | 52 | UGUUGGGGUCCUGGGGCAC | 2950 | GUGCCCCAGGACCCCAACA |
| siRNA 53 | 53 | GUUGGGGUCCUGGGGCACC | 2951 | GGUGCCCCAGGACCCCAAC |
| siRNA 54 | 54 | UUGGGGUCCUGGGGCACCU | 2952 | AGGUGCCCCAGGACCCCAA |
| siRNA 55 | 55 | UGGGGUCCUGGGGCACCUG | 2953 | CAGGUGCCCCAGGACCCCA |
| siRNA 56 | 56 | GGGGUCCUGGGGCACCUGC | 2954 | GCAGGUGCCCCAGGACCCC |
| siRNA 57 | 57 | GGGUCCUGGGGCACCUGCC | 2955 | GGCAGGUGCCCCAGGACCC |
| siRNA 58 | 58 | GGUCCUGGGGCACCUGCCU | 2956 | AGGCAGGUGCCCCAGGACC |
| siRNA 59 | 59 | GUCCUGGGGCACCUGCCUU | 2957 | AAGGCAGGUGCCCCAGGAC |
| siRNA 60 | 60 | UCCUGGGGCACCUGCCUUA | 2958 | UAAGGCAGGUGCCCCAGGA |
| SIRNA 61 | 61 | CCUGGGGCACCUGCCUUAC | 2959 | GUAAGGCAGGUGCCCCAGG |
| siRNA 62 | 62 | CUGGGGCACCUGCCUUACA | 2960 | UGUAAGGCAGGUGCCCCAG |
| siRNA 63 | 63 | UGGGGCACCUGCCUUACAU | 2961 | AUGUAAGGCAGGUGCCCCA |
| siRNA 64 | 64 | GGGGCACCUGCCUUACAUG | 2962 | CAUGUAAGGCAGGUGCCCC |
| siRNA 65 | 65 | GGGCACCUGCCUUACAUGG | 2963 | CCAUGUAAGGCAGGUGCCC |
| siRNA 66 | 66 | GGCACCUGCCUUACAUGGC | 2964 | GCCAUGUAAGGCAGGUGCC |
| siRNA 67 | 67 | GCACCUGCCUUACAUGGCU | 2965 | AGCCAUGUAAGGCAGGUGC |
| siRNA 68 | 68 | CACCUGCCUUACAUGGCUU | 2966 | AAGCCAUGUAAGGCAGGUG |
| siRNA 69 | 69 | ACCUGCCUUACAUGGCUUG | 2967 | CAAGCCAUGUAAGGCAGGU |
| siRNA 70 | 70 | CCUGCCUUACAUGGCUUGU | 2968 | ACAAGCCAUGUAAGGCAGG |
| siRNA 71 | 71 | CUGCCUUACAUGGCUUGUU | 2969 | AACAAGCCAUGUAAGGCAG |
| siRNA 72 | 72 | UGCCUUACAUGGCUUGUUU | 2970 | AAACAAGCCAUGUAAGGCA |
| siRNA 73 | 73 | GCCUUACAUGGCUUGUUUA | 2971 | UAAACAAGCCAUGUAAGGC |
| siRNA 74 | 74 | CCUUACAUGGCUUGUUUAU | 2972 | AUAAACAAGCCAUGUAAGG |
| siRNA 75 | 75 | CUUACAUGGCUUGUUUAUG | 2973 | CAUAAACAAGCCAUGUAAG |
| siRNA 76 | 76 | UUACAUGGCUUGUUUAUGA | 2974 | UCAUAAACAAGCCAUGUAA |
| siRNA 77 | 77 | UACAUGGCUUGUUUAUGAA | 2975 | UUCAUAAACAAGCCAUGUA |
| siRNA 78 | 78 | ACAUGGCUUGUUUAUGAAC | 2976 | GUUCAUAAACAAGCCAUGU |
| siRNA 79 | 79 | CAUGGCUUGUUUAUGAACA | 2977 | UGUUCAUAAACAAGCCAUG |
| siRNA 80 | 80 | AUGGCUUGUUUAUGAACAU | 2978 | AUGUUCAUAAACAAGCCAU |
| siRNA 81 | 81 | UGGCUUGUUUAUGAACAUU | 2979 | AAUGUUCAUAAACAAGCCA |
| siRNA 82 | 82 | GGCUUGUUUAUGAACAUUA | 2980 | UAAUGUUCAUAAACAAGCC |
| siRNA 83 | 83 | GCUUGUUUAUGAACAUUAA | 2981 | UUAAUGUUCAUAAACAAGC |
| siRNA 84 | 84 | CUUGUUUAUGAACAUUAAA | 2982 | UUUAAUGUUCAUAAACAAG |
| siRNA 85 | 85 | UUGUUUAUGAACAUUAAAG | 2983 | CUUUAAUGUUCAUAAACAA |
| siRNA 86 | 86 | UGUUUAUGAACAUUAAAGG | 2984 | CCUUUAAUGUUCAUAAACA |
| siRNA 87 | 87 | GUUUAUGAACAUUAAAGGG | 2985 | CCCUUUAAUGUUCAUAAAC |
| siRNA 88 | 88 | UUUAUGAACAUUAAAGGGA | 2986 | UCCCUUUAAUGUUCAUAAA |
| siRNA 89 | 89 | UUAUGAACAUUAAAGGGAA | 2987 | UUCCCUUUAAUGUUCAUAA |
| siRNA 90 | 90 | UAUGAACAUUAAAGGGAAG | 2988 | CUUCCCUUUAAUGUUCAUA |
| siRNA 91 | 91 | AUGAACAUUAAAGGGAAGA | 2989 | UCUUCCCUUUAAUGUUCAU |
| siRNA 92 | 92 | UGAACAUUAAAGGGAAGAA | 2990 | UUCUUCCCUUUAAUGUUCA |
| siRNA 93 | 93 | GAACAUUAAAGGGAAGAAG | 2991 | CUUCUUCCCUUUAAUGUUC |
| siRNA 94 | 94 | AACAUUAAAGGGAAGAAGU | 2992 | ACUUCUUCCCUUUAAUGUU |
| siRNA 95 | 95 | ACAUUAAAGGGAAGAAGUU | 2993 | AACUUCUUCCCUUUAAUGU |
| siRNA 96 | 96 | CAUUAAAGGGAAGAAGUUG | 2994 | CAACUUCUUCCCUUUAAUG |
| siRNA 97 | 97 | AUUAAAGGGAAGAAGUUGA | 2995 | UCAACUUCUUCCCUUUAAU |
| siRNA 98 | 98 | UUAAAGGGAAGAAGUUGAA | 2996 | UUCAACUUCUUCCCUUUAA |
| siRNA 99 | 99 | UAAAGGGAAGAAGUUGAAG | 2997 | CUUCAACUUCUUCCCUUUA |
| siRNA 100 | 100 | AAAGGGAAGAAGUUGAAGC | 2998 | GCUUCAACUUCUUCCCUUU |
| siRNA 101 | 101 | AAGGGAAGAAGUUGAAGCU | 2999 | AGCUUCAACUUCUUCCCUU |
| siRNA 102 | 102 | AGGGAAGAAGUUGAAGCUU | 3000 | AAGCUUCAACUUCUUCCCU |
| siRNA 103 | 103 | GGGAAGAAGUUGAAGCUUG | 3001 | CAAGCUUCAACUUCUUCCC |
| siRNA 104 | 104 | GGAAGAAGUUGAAGCUUGA | 3002 | UCAAGCUUCAACUUCUUCC |
| siRNA 105 | 105 | GAAGAAGUUGAAGCUUGAG | 3003 | CUCAAGCUUCAACUUCUUC |
| siRNA 106 | 106 | AAGAAGUUGAAGCUUGAGG | 3004 | CCUCAAGCUUCAACUUCUU |
| siRNA 107 | 107 | AGAAGUUGAAGCUUGAGGA | 3005 | UCCUCAAGCUUCAACUUCU |
| siRNA 108 | 108 | GAAGUUGAAGCUUGAGGAG | 3006 | CUCCUCAAGCUUCAACUUC |
| siRNA 109 | 109 | AAGUUGAAGCUUGAGGAGC | 3007 | GCUCCUCAAGCUUCAACUU |
| siRNA 110 | 110 | AGUUGAAGCUUGAGGAGCG | 3008 | CGCUCCUCAAGCUUCAACU |
| siRNA 111 | 111 | GUUGAAGCUUGAGGAGCGA | 3009 | UCGCUCCUCAAGCUUCAAC |
| siRNA 112 | 112 | UUGAAGCUUGAGGAGCGAG | 3010 | CUCGCUCCUCAAGCUUCAA |
| siRNA 113 | 113 | UGAAGCUUGAGGAGCGAGG | 3011 | CCUCGCUCCUCAAGCUUCA |
| siRNA 114 | 114 | GAAGCUUGAGGAGCGAGGA | 3012 | UCCUCGCUCCUCAAGCUUC |
| siRNA 115 | 115 | AAGCUUGAGGAGCGAGGAU | 3013 | AUCCUCGCUCCUCAAGCUU |
| siRNA 116 | 116 | AGCUUGAGGAGCGAGGAUG | 3014 | CAUCCUCGCUCCUCAAGCU |
| siRNA 117 | 117 | GCUUGAGGAGCGAGGAUGG | 3015 | CCAUCCUCGCUCCUCAAGC |
| siRNA 118 | 118 | CUUGAGGAGCGAGGAUGGC | 3016 | GCCAUCCUCGCUCCUCAAG |
| siRNA 119 | 119 | UUGAGGAGCGAGGAUGGCA | 3017 | UGCCAUCCUCGCUCCUCAA |
| siRNA 120 | 120 | UGAGGAGCGAGGAUGGCAG | 3018 | CUGCCAUCCUCGCUCCUCA |
| siRNA 121 | 121 | GAGGAGCGAGGAUGGCAGU | 3019 | ACUGCCAUCCUCGCUCCUC |
| siRNA 122 | 122 | AGGAGCGAGGAUGGCAGUC | 3020 | GACUGCCAUCCUCGCUCCU |
| siRNA 123 | 123 | GGAGCGAGGAUGGCAGUCA | 3021 | UGACUGCCAUCCUCGCUCC |
| siRNA 124 | 124 | GAGCGAGGAUGGCAGUCAA | 3022 | UUGACUGCCAUCCUCGCUC |
| siRNA 125 | 125 | AGCGAGGAUGGCAGUCAAC | 3023 | GUUGACUGCCAUCCUCGCU |
| siRNA 126 | 126 | GCGAGGAUGGCAGUCAACA | 3024 | UGUUGACUGCCAUCCUCGC |
| siRNA 127 | 127 | CGAGGAUGGCAGUCAACAA | 3025 | UUGUUGACUGCCAUCCUCG |
| siRNA 128 | 128 | GAGGAUGGCAGUCAACAAA | 3026 | UUUGUUGACUGCCAUCCUC |
| siRNA 129 | 129 | AGGAUGGCAGUCAACAAAG | 3027 | CUUUGUUGACUGCCAUCCU |
| siRNA 130 | 130 | GGAUGGCAGUCAACAAAGG | 3028 | CCUUUGUUGACUGCCAUCC |
| siRNA 131 | 131 | GAUGGCAGUCAACAAAGGC | 3029 | GCCUUUGUUGACUGCCAUC |
| siRNA 132 | 132 | AUGGCAGUCAACAAAGGCC | 3030 | GGCCUUUGUUGACUGCCAU |
| siRNA 133 | 133 | UGGCAGUCAACAAAGGCCU | 3031 | AGGCCUUUGUUGACUGCCA |
| siRNA 134 | 134 | GGCAGUCAACAAAGGCCUC | 3032 | GAGGCCUUUGUUGACUGCC |
| siRNA 135 | 135 | GCAGUCAACAAAGGCCUCA | 3033 | UGAGGCCUUUGUUGACUGC |
| siRNA 136 | 136 | CAGUCAACAAAGGCCUCAC | 3034 | GUGAGGCCUUUGUUGACUG |
| siRNA 137 | 137 | AGUCAACAAAGGCCUCACC | 3035 | GGUGAGGCCUUUGUUGACU |
| siRNA 138 | 138 | GUCAACAAAGGCCUCACCU | 3036 | AGGUGAGGCCUUUGUUGAC |
| siRNA 139 | 139 | UCAACAAAGGCCUCACCUU | 3037 | AAGGUGAGGCCUUUGUUGA |
| siRNA 140 | 140 | CAACAAAGGCCUCACCUUG | 3038 | CAAGGUGAGGCCUUUGUUG |
| siRNA 141 | 141 | AACAAAGGCCUCACCUUGC | 3039 | GCAAGGUGAGGCCUUUGUU |
| siRNA 142 | 142 | ACAAAGGCCUCACCUUGCU | 3040 | AGCAAGGUGAGGCCUUUGU |
| siRNA 143 | 143 | CAAAGGCCUCACCUUGCUG | 3041 | CAGCAAGGUGAGGCCUUUG |
| siRNA 144 | 144 | AAAGGCCUCACCUUGCUGG | 3042 | CCAGCAAGGUGAGGCCUUU |
| siRNA 145 | 145 | AAGGCCUCACCUUGCUGGA | 3043 | UCCAGCAAGGUGAGGCCUU |
| siRNA 146 | 146 | AGGCCUCACCUUGCUGGAU | 3044 | AUCCAGCAAGGUGAGGCCU |
| siRNA 147 | 147 | GGCCUCACCUUGCUGGAUG | 3045 | CAUCCAGCAAGGUGAGGCC |
| siRNA 148 | 148 | GCCUCACCUUGCUGGAUGG | 3046 | CCAUCCAGCAAGGUGAGGC |
| siRNA 149 | 149 | CCUCACCUUGCUGGAUGGA | 3047 | UCCAUCCAGCAAGGUGAGG |
| siRNA 150 | 150 | CUCACCUUGCUGGAUGGAG | 3048 | CUCCAUCCAGCAAGGUGAG |
| siRNA 151 | 151 | UCACCUUGCUGGAUGGAGA | 3049 | UCUCCAUCCAGCAAGGUGA |
| siRNA 152 | 152 | CACCUUGCUGGAUGGAGAC | 3050 | GUCUCCAUCCAGCAAGGUG |
| siRNA 153 | 153 | ACCUUGCUGGAUGGAGACC | 3051 | GGUCUCCAUCCAGCAAGGU |
| siRNA 154 | 154 | CCUUGCUGGAUGGAGACCU | 3052 | AGGUCUCCAUCCAGCAAGG |
| siRNA 155 | 155 | CUUGCUGGAUGGAGACCUC | 3053 | GAGGUCUCCAUCCAGCAAG |
| siRNA 156 | 156 | UUGCUGGAUGGAGACCUCC | 3054 | GGAGGUCUCCAUCCAGCAA |
| siRNA 157 | 157 | UGCUGGAUGGAGACCUCCC | 3055 | GGGAGGUCUCCAUCCAGCA |
| siRNA 158 | 158 | GCUGGAUGGAGACCUCCCU | 3056 | AGGGAGGUCUCCAUCCAGC |
| siRNA 159 | 159 | CUGGAUGGAGACCUCCCUG | 3057 | CAGGGAGGUCUCCAUCCAG |
| siRNA 160 | 160 | UGGAUGGAGACCUCCCUGA | 3058 | UCAGGGAGGUCUCCAUCCA |
| siRNA 161 | 161 | GGAUGGAGACCUCCCUGAG | 3059 | CUCAGGGAGGUCUCCAUCC |
| siRNA 162 | 162 | GAUGGAGACCUCCCUGAGC | 3060 | GCUCAGGGAGGUCUCCAUC |
| siRNA 163 | 163 | AUGGAGACCUCCCUGAGCA | 3061 | UGCUCAGGGAGGUCUCCAU |
| siRNA 164 | 164 | UGGAGACCUCCCUGAGCAG | 3062 | CUGCUCAGGGAGGUCUCCA |
| siRNA 165 | 165 | GGAGACCUCCCUGAGCAGG | 3063 | CCUGCUCAGGGAGGUCUCC |
| siRNA 166 | 166 | GAGACCUCCCUGAGCAGGA | 3064 | UCCUGCUCAGGGAGGUCUC |
| siRNA 167 | 167 | AGACCUCCCUGAGCAGGAG | 3065 | CUCCUGCUCAGGGAGGUCU |
| siRNA 168 | 168 | GACCUCCCUGAGCAGGAGA | 3066 | UCUCCUGCUCAGGGAGGUC |
| siRNA 169 | 169 | ACCUCCCUGAGCAGGAGAA | 3067 | UUCUCCUGCUCAGGGAGGU |
| siRNA 170 | 170 | CCUCCCUGAGCAGGAGAAU | 3068 | AUUCUCCUGCUCAGGGAGG |
| siRNA 171 | 171 | CUCCCUGAGCAGGAGAAUG | 3069 | CAUUCUCCUGCUCAGGGAG |
| siRNA 172 | 172 | UCCCUGAGCAGGAGAAUGU | 3070 | ACAUUCUCCUGCUCAGGGA |
| siRNA 173 | 173 | CCCUGAGCAGGAGAAUGUG | 3071 | CACAUUCUCCUGCUCAGGG |
| siRNA 174 | 174 | CCUGAGCAGGAGAAUGUGC | 3072 | GCACAUUCUCCUGCUCAGG |
| siRNA 175 | 175 | CUGAGCAGGAGAAUGUGCU | 3073 | AGCACAUUCUCCUGCUCAG |
| siRNA 176 | 176 | UGAGCAGGAGAAUGUGCUG | 3074 | CAGCACAUUCUCCUGCUCA |
| siRNA 177 | 177 | GAGCAGGAGAAUGUGCUGC | 3075 | GCAGCACAUUCUCCUGCUC |
| siRNA 178 | 178 | AGCAGGAGAAUGUGCUGCA | 3076 | UGCAGCACAUUCUCCUGCU |
| siRNA 179 | 179 | GCAGGAGAAUGUGCUGCAG | 3077 | CUGCAGCACAUUCUCCUGC |
| siRNA 180 | 180 | CAGGAGAAUGUGCUGCAGC | 3078 | GCUGCAGCACAUUCUCCUG |
| siRNA 181 | 181 | AGGAGAAUGUGCUGCAGCG | 3079 | CGCUGCAGCACAUUCUCCU |
| siRNA 182 | 182 | GGAGAAUGUGCUGCAGCGG | 3080 | CCGCUGCAGCACAUUCUCC |
| siRNA 183 | 183 | GAGAAUGUGCUGCAGCGGG | 3081 | CCCGCUGCAGCACAUUCUC |
| siRNA 184 | 184 | AGAAUGUGCUGCAGCGGGU | 3082 | ACCCGCUGCAGCACAUUCU |
| siRNA 185 | 185 | GAAUGUGCUGCAGCGGGUC | 3083 | GACCCGCUGCAGCACAUUC |
| siRNA 186 | 186 | AAUGUGCUGCAGCGGGUCC | 3084 | GGACCCGCUGCAGCACAUU |
| siRNA 187 | 187 | AUGUGCUGCAGCGGGUCCU | 3085 | AGGACCCGCUGCAGCACAU |
| siRNA 188 | 188 | UGUGCUGCAGCGGGUCCUG | 3086 | CAGGACCCGCUGCAGCACA |
| siRNA 189 | 189 | GUGCUGCAGCGGGUCCUGC | 3087 | GCAGGACCCGCUGCAGCAC |
| siRNA 190 | 190 | UGCUGCAGCGGGUCCUGCA | 3088 | UGCAGGACCCGCUGCAGCA |
| siRNA 191 | 191 | GCUGCAGCGGGUCCUGCAG | 3089 | CUGCAGGACCCGCUGCAGC |
| siRNA 192 | 192 | CUGCAGCGGGUCCUGCAGC | 3090 | GCUGCAGGACCCGCUGCAG |
| siRNA 193 | 193 | UGCAGCGGGUCCUGCAGCU | 3091 | AGCUGCAGGACCCGCUGCA |
| siRNA 194 | 194 | GCAGCGGGUCCUGCAGCUG | 3092 | CAGCUGCAGGACCCGCUGC |
| siRNA 195 | 195 | CAGCGGGUCCUGCAGCUGC | 3093 | GCAGCUGCAGGACCCGCUG |
| siRNA 196 | 196 | AGCGGGUCCUGCAGCUGCC | 3094 | GGCAGCUGCAGGACCCGCU |
| siRNA 197 | 197 | GCGGGUCCUGCAGCUGCCG | 3095 | CGGCAGCUGCAGGACCCGC |
| siRNA 198 | 198 | CGGGUCCUGCAGCUGCCGG | 3096 | CCGGCAGCUGCAGGACCCG |
| siRNA 199 | 199 | GGGUCCUGCAGCUGCCGGU | 3097 | ACCGGCAGCUGCAGGACCC |
| siRNA 200 | 200 | GGUCCUGCAGCUGCCGGUG | 3098 | CACCGGCAGCUGCAGGACC |
| siRNA 201 | 201 | GUCCUGCAGCUGCCGGUGG | 3099 | CCACCGGCAGCUGCAGGAC |
| siRNA 202 | 202 | UCCUGCAGCUGCCGGUGGU | 3100 | ACCACCGGCAGCUGCAGGA |
| siRNA 203 | 203 | CCUGCAGCUGCCGGUGGUG | 3101 | CACCACCGGCAGCUGCAGG |
| siRNA 204 | 204 | CUGCAGCUGCCGGUGGUGA | 3102 | UCACCACCGGCAGCUGCAG |
| siRNA 205 | 205 | UGCAGCUGCCGGUGGUGAG | 3103 | CUCACCACCGGCAGCUGCA |
| siRNA 206 | 206 | GCAGCUGCCGGUGGUGAGU | 3104 | ACUCACCACCGGCAGCUGC |
| siRNA 207 | 207 | CAGCUGCCGGUGGUGAGUG | 3105 | CACUCACCACCGGCAGCUG |
| siRNA 208 | 208 | AGCUGCCGGUGGUGAGUGG | 3106 | CCACUCACCACCGGCAGCU |
| siRNA 209 | 209 | GCUGCCGGUGGUGAGUGGC | 3107 | GCCACUCACCACCGGCAGC |
| siRNA 210 | 210 | CUGCCGGUGGUGAGUGGCA | 3108 | UGCCACUCACCACCGGCAG |
| siRNA 211 | 211 | UGCCGGUGGUGAGUGGCAC | 3109 | GUGCCACUCACCACCGGCA |
| siRNA 212 | 212 | GCCGGUGGUGAGUGGCACC | 3110 | GGUGCCACUCACCACCGGC |
| siRNA 213 | 213 | CCGGUGGUGAGUGGCACCU | 3111 | AGGUGCCACUCACCACCGG |
| siRNA 214 | 214 | CGGUGGUGAGUGGCACCUG | 3112 | CAGGUGCCACUCACCACCG |
| siRNA 215 | 215 | GGUGGUGAGUGGCACCUGC | 3113 | GCAGGUGCCACUCACCACC |
| siRNA 216 | 216 | GUGGUGAGUGGCACCUGCG | 3114 | CGCAGGUGCCACUCACCAC |
| siRNA 217 | 217 | UGGUGAGUGGCACCUGCGA | 3115 | UCGCAGGUGCCACUCACCA |
| siRNA 218 | 218 | GGUGAGUGGCACCUGCGAA | 3116 | UUCGCAGGUGCCACUCACC |
| siRNA 219 | 219 | GUGAGUGGCACCUGCGAAU | 3117 | AUUCGCAGGUGCCACUCAC |
| siRNA 220 | 220 | UGAGUGGCACCUGCGAAUG | 3118 | CAUUCGCAGGUGCCACUCA |
| siRNA 221 | 221 | GAGUGGCACCUGCGAAUGC | 3119 | GCAUUCGCAGGUGCCACUC |
| siRNA 222 | 222 | AGUGGCACCUGCGAAUGCU | 3120 | AGCAUUCGCAGGUGCCACU |
| siRNA 223 | 223 | GUGGCACCUGCGAAUGCUU | 3121 | AAGCAUUCGCAGGUGCCAC |
| siRNA 224 | 224 | UGGCACCUGCGAAUGCUUC | 3122 | GAAGCAUUCGCAGGUGCCA |
| siRNA 225 | 225 | GGCACCUGCGAAUGCUUCC | 3123 | GGAAGCAUUCGCAGGUGCC |
| siRNA 226 | 226 | GCACCUGCGAAUGCUUCCA | 3124 | UGGAAGCAUUCGCAGGUGC |
| siRNA 227 | 227 | CACCUGCGAAUGCUUCCAG | 3125 | CUGGAAGCAUUCGCAGGUG |
| siRNA 228 | 228 | ACCUGCGAAUGCUUCCAGA | 3126 | UCUGGAAGCAUUCGCAGGU |
| siRNA 229 | 229 | CCUGCGAAUGCUUCCAGAA | 3127 | UUCUGGAAGCAUUCGCAGG |
| siRNA 230 | 230 | CUGCGAAUGCUUCCAGAAG | 3128 | CUUCUGGAAGCAUUCGCAG |
| siRNA 231 | 231 | UGCGAAUGCUUCCAGAAGA | 3129 | UCUUCUGGAAGCAUUCGCA |
| siRNA 232 | 232 | GCGAAUGCUUCCAGAAGAC | 3130 | GUCUUCUGGAAGCAUUCGC |
| siRNA 233 | 233 | CGAAUGCUUCCAGAAGACC | 3131 | GGUCUUCUGGAAGCAUUCG |
| siRNA 234 | 234 | GAAUGCUUCCAGAAGACCU | 3132 | AGGUCUUCUGGAAGCAUUC |
| siRNA 235 | 235 | AAUGCUUCCAGAAGACCUA | 3133 | UAGGUCUUCUGGAAGCAUU |
| siRNA 236 | 236 | AUGCUUCCAGAAGACCUAC | 3134 | GUAGGUCUUCUGGAAGCAU |
| siRNA 237 | 237 | UGCUUCCAGAAGACCUACA | 3135 | UGUAGGUCUUCUGGAAGCA |
| siRNA 238 | 238 | GCUUCCAGAAGACCUACAC | 3136 | GUGUAGGUCUUCUGGAAGC |
| siRNA 239 | 239 | CUUCCAGAAGACCUACACC | 3137 | GGUGUAGGUCUUCUGGAAG |
| siRNA 240 | 240 | UUCCAGAAGACCUACACCA | 3138 | UGGUGUAGGUCUUCUGGAA |
| siRNA 241 | 241 | UCCAGAAGACCUACACCAG | 3139 | CUGGUGUAGGUCUUCUGGA |
| siRNA 242 | 242 | CCAGAAGACCUACACCAGC | 3140 | GCUGGUGUAGGUCUUCUGG |
| siRNA 243 | 243 | CAGAAGACCUACACCAGCA | 3141 | UGCUGGUGUAGGUCUUCUG |
| siRNA 244 | 244 | AGAAGACCUACACCAGCAC | 3142 | GUGCUGGUGUAGGUCUUCU |
| siRNA 245 | 245 | GAAGACCUACACCAGCACU | 3143 | AGUGCUGGUGUAGGUCUUC |
| siRNA 246 | 246 | AAGACCUACACCAGCACUA | 3144 | UAGUGCUGGUGUAGGUCUU |
| siRNA 247 | 247 | AGACCUACACCAGCACUAA | 3145 | UUAGUGCUGGUGUAGGUCU |
| siRNA 248 | 248 | GACCUACACCAGCACUAAG | 3146 | CUUAGUGCUGGUGUAGGUC |
| siRNA 249 | 249 | ACCUACACCAGCACUAAGG | 3147 | CCUUAGUGCUGGUGUAGGU |
| siRNA 250 | 250 | CCUACACCAGCACUAAGGA | 3148 | UCCUUAGUGCUGGUGUAGG |
| siRNA 251 | 251 | CUACACCAGCACUAAGGAA | 3149 | UUCCUUAGUGCUGGUGUAG |
| siRNA 252 | 252 | UACACCAGCACUAAGGAAG | 3150 | CUUCCUUAGUGCUGGUGUA |
| siRNA 253 | 253 | ACACCAGCACUAAGGAAGC | 3151 | GCUUCCUUAGUGCUGGUGU |
| siRNA 254 | 254 | CACCAGCACUAAGGAAGCC | 3152 | GGCUUCCUUAGUGCUGGUG |
| siRNA 255 | 255 | ACCAGCACUAAGGAAGCCC | 3153 | GGGCUUCCUUAGUGCUGGU |
| siRNA 256 | 256 | CCAGCACUAAGGAAGCCCA | 3154 | UGGGCUUCCUUAGUGCUGG |
| siRNA 257 | 257 | CAGCACUAAGGAAGCCCAC | 3155 | GUGGGCUUCCUUAGUGCUG |
| siRNA 258 | 258 | AGCACUAAGGAAGCCCACC | 3156 | GGUGGGCUUCCUUAGUGCU |
| siRNA 259 | 259 | GCACUAAGGAAGCCCACCC | 3157 | GGGUGGGCUUCCUUAGUGC |
| siRNA 260 | 260 | CACUAAGGAAGCCCACCCC | 3158 | GGGGUGGGCUUCCUUAGUG |
| siRNA 261 | 261 | ACUAAGGAAGCCCACCCCC | 3159 | GGGGGUGGGCUUCCUUAGU |
| siRNA 262 | 262 | CUAAGGAAGCCCACCCCCU | 3160 | AGGGGGUGGGCUUCCUUAG |
| siRNA 263 | 263 | UAAGGAAGCCCACCCCCUG | 3161 | CAGGGGGUGGGCUUCCUUA |
| siRNA 264 | 264 | AAGGAAGCCCACCCCCUGG | 3162 | CCAGGGGGUGGGCUUCCUU |
| siRNA 265 | 265 | AGGAAGCCCACCCCCUGGU | 3163 | ACCAGGGGGUGGGCUUCCU |
| siRNA 266 | 266 | GGAAGCCCACCCCCUGGUG | 3164 | CACCAGGGGGUGGGCUUCC |
| siRNA 267 | 267 | GAAGCCCACCCCCUGGUGG | 3165 | CCACCAGGGGGUGGGCUUC |
| siRNA 268 | 268 | AAGCCCACCCCCUGGUGGC | 3166 | GCCACCAGGGGGUGGGCUU |
| siRNA 269 | 269 | AGCCCACCCCCUGGUGGCC | 3167 | GGCCACCAGGGGGUGGGCU |
| siRNA 270 | 270 | GCCCACCCCCUGGUGGCCU | 3168 | AGGCCACCAGGGGGUGGGC |
| siRNA 271 | 271 | CCCACCCCCUGGUGGCCUC | 3169 | GAGGCCACCAGGGGGGGG |
| siRNA 272 | 272 | CCACCCCCUGGUGGCCUCU | 3170 | AGAGGCCACCAGGGGGUGG |
| siRNA 273 | 273 | CACCCCCUGGUGGCCUCUG | 3171 | CAGAGGCCACCAGGGGGUG |
| siRNA 274 | 274 | ACCCCCUGGUGGCCUCUGU | 3172 | ACAGAGGCCACCAGGGGGU |
| siRNA 275 | 275 | CCCCCUGGUGGCCUCUGUG | 3173 | CACAGAGGCCACCAGGGGG |
| siRNA 276 | 276 | CCCCUGGUGGCCUCUGUGU | 3174 | ACACAGAGGCCACCAGGGG |
| siRNA 277 | 277 | CCCUGGUGGCCUCUGUGUG | 3175 | CACACAGAGGCCACCAGGG |
| siRNA 278 | 278 | CCUGGUGGCCUCUGUGUGC | 3176 | GCACACAGAGGCCACCAGG |
| siRNA 279 | 279 | CUGGUGGCCUCUGUGUGCA | 3177 | UGCACACAGAGGCCACCAG |
| siRNA 280 | 280 | UGGUGGCCUCUGUGUGCAA | 3178 | UUGCACACAGAGGCCACCA |
| siRNA 281 | 281 | GGUGGCCUCUGUGUGCAAU | 3179 | AUUGCACACAGAGGCCACC |
| siRNA 282 | 282 | GUGGCCUCUGUGUGCAAUG | 3180 | CAUUGCACACAGAGGCCAC |
| siRNA 283 | 283 | UGGCCUCUGUGUGCAAUGC | 3181 | GCAUUGCACACAGAGGCCA |
| siRNA 284 | 284 | GGCCUCUGUGUGCAAUGCC | 3182 | GGCAUUGCACACAGAGGCC |
| siRNA 285 | 285 | GCCUCUGUGUGCAAUGCCU | 3183 | AGGCAUUGCACACAGAGGC |
| siRNA 286 | 286 | CCUCUGUGUGCAAUGCCUA | 3184 | UAGGCAUUGCACACAGAGG |
| siRNA 287 | 287 | CUCUGUGUGCAAUGCCUAU | 3185 | AUAGGCAUUGCACACAGAG |
| siRNA 288 | 288 | UCUGUGUGCAAUGCCUAUG | 3186 | CAUAGGCAUUGCACACAGA |
| siRNA 289 | 289 | CUGUGUGCAAUGCCUAUGA | 3187 | UCAUAGGCAUUGCACACAG |
| siRNA 290 | 290 | UGUGUGCAAUGCCUAUGAG | 3188 | CUCAUAGGCAUUGCACACA |
| siRNA 291 | 291 | GUGUGCAAUGCCUAUGAGA | 3189 | UCUCAUAGGCAUUGCACAC |
| siRNA 292 | 292 | UGUGCAAUGCCUAUGAGAA | 3190 | UUCUCAUAGGCAUUGCACA |
| siRNA 293 | 293 | GUGCAAUGCCUAUGAGAAG | 3191 | CUUCUCAUAGGCAUUGCAC |
| siRNA 294 | 294 | UGCAAUGCCUAUGAGAAGG | 3192 | CCUUCUCAUAGGCAUUGCA |
| siRNA 295 | 295 | GCAAUGCCUAUGAGAAGGG | 3193 | CCCUUCUCAUAGGCAUUGC |
| siRNA 296 | 296 | CAAUGCCUAUGAGAAGGGC | 3194 | GCCCUUCUCAUAGGCAUUG |
| siRNA 297 | 297 | AAUGCCUAUGAGAAGGGCG | 3195 | CGCCCUUCUCAUAGGCAUU |
| siRNA 298 | 298 | AUGCCUAUGAGAAGGGCGU | 3196 | ACGCCCUUCUCAUAGGCAU |
| siRNA 299 | 299 | UGCCUAUGAGAAGGGCGUG | 3197 | CACGCCCUUCUCAUAGGCA |
| siRNA 300 | 300 | GCCUAUGAGAAGGGCGUGC | 3198 | GCACGCCCUUCUCAUAGGC |
| siRNA 301 | 301 | CCUAUGAGAAGGGCGUGCA | 3199 | UGCACGCCCUUCUCAUAGG |
| siRNA 302 | 302 | CUAUGAGAAGGGCGUGCAG | 3200 | CUGCACGCCCUUCUCAUAG |
| siRNA 303 | 303 | UAUGAGAAGGGCGUGCAGA | 3201 | UCUGCACGCCCUUCUCAUA |
| siRNA 304 | 304 | AUGAGAAGGGCGUGCAGAG | 3202 | CUCUGCACGCCCUUCUCAU |
| siRNA 305 | 305 | UGAGAAGGGCGUGCAGAGC | 3203 | GCUCUGCACGCCCUUCUCA |
| siRNA 306 | 306 | GAGAAGGGCGUGCAGAGCG | 3204 | CGCUCUGCACGCCCUUCUC |
| siRNA 307 | 307 | AGAAGGGCGUGCAGAGCGC | 3205 | GCGCUCUGCACGCCCUUCU |
| siRNA 308 | 308 | GAAGGGCGUGCAGAGCGCC | 3206 | GGCGCUCUGCACGCCCUUC |
| siRNA 309 | 309 | AAGGGCGUGCAGAGCGCCA | 3207 | UGGCGCUCUGCACGCCCUU |
| siRNA 310 | 310 | AGGGCGUGCAGAGCGCCAG | 3208 | CUGGCGCUCUGCACGCCCU |
| siRNA 311 | 311 | GGGCGUGCAGAGCGCCAGU | 3209 | ACUGGCGCUCUGCACGCCC |
| siRNA 312 | 312 | GGCGUGCAGAGCGCCAGUA | 3210 | UACUGGCGCUCUGCACGCC |
| siRNA 313 | 313 | GCGUGCAGAGCGCCAGUAG | 3211 | CUACUGGCGCUCUGCACGC |
| siRNA 314 | 314 | CGUGCAGAGCGCCAGUAGC | 3212 | GCUACUGGCGCUCUGCACG |
| siRNA 315 | 315 | GUGCAGAGCGCCAGUAGCU | 3213 | AGCUACUGGCGCUCUGCAC |
| siRNA 316 | 316 | UGCAGAGCGCCAGUAGCUU | 3214 | AAGCUACUGGCGCUCUGCA |
| siRNA 317 | 317 | GCAGAGCGCCAGUAGCUUG | 3215 | CAAGCUACUGGCGCUCUGC |
| siRNA 318 | 318 | CAGAGCGCCAGUAGCUUGG | 3216 | CCAAGCUACUGGCGCUCUG |
| siRNA 319 | 319 | AGAGCGCCAGUAGCUUGGC | 3217 | GCCAAGCUACUGGCGCUCU |
| siRNA 320 | 320 | GAGCGCCAGUAGCUUGGCU | 3218 | AGCCAAGCUACUGGCGCUC |
| siRNA 321 | 321 | AGCGCCAGUAGCUUGGCUG | 3219 | CAGCCAAGCUACUGGCGCU |
| siRNA 322 | 322 | GCGCCAGUAGCUUGGCUGC | 3220 | GCAGCCAAGCUACUGGCGC |
| siRNA 323 | 323 | CGCCAGUAGCUUGGCUGCC | 3221 | GGCAGCCAAGCUACUGGCG |
| siRNA 324 | 324 | GCCAGUAGCUUGGCUGCCU | 3222 | AGGCAGCCAAGCUACUGGC |
| siRNA 325 | 325 | CCAGUAGCUUGGCUGCCUG | 3223 | CAGGCAGCCAAGCUACUGG |
| siRNA 326 | 326 | CAGUAGCUUGGCUGCCUGG | 3224 | CCAGGCAGCCAAGCUACUG |
| siRNA 327 | 327 | AGUAGCUUGGCUGCCUGGA | 3225 | UCCAGGCAGCCAAGCUACU |
| siRNA 328 | 328 | GUAGCUUGGCUGCCUGGAG | 3226 | CUCCAGGCAGCCAAGCUAC |
| siRNA 329 | 329 | UAGCUUGGCUGCCUGGAGC | 3227 | GCUCCAGGCAGCCAAGCUA |
| siRNA 330 | 330 | AGCUUGGCUGCCUGGAGCA | 3228 | UGCUCCAGGCAGCCAAGCU |
| siRNA 331 | 331 | GCUUGGCUGCCUGGAGCAU | 3229 | AUGCUCCAGGCAGCCAAGC |
| SiRNA 332 | 332 | CUUGGCUGCCUGGAGCAUG | 3230 | CAUGCUCCAGGCAGCCAAG |
| siRNA 333 | 333 | UUGGCUGCCUGGAGCAUGG | 3231 | CCAUGCUCCAGGCAGCCAA |
| siRNA 334 | 334 | UGGCUGCCUGGAGCAUGGA | 3232 | UCCAUGCUCCAGGCAGCCA |
| siRNA 335 | 335 | GGCUGCCUGGAGCAUGGAG | 3233 | CUCCAUGCUCCAGGCAGCC |
| siRNA 336 | 336 | GCUGCCUGGAGCAUGGAGC | 3234 | GCUCCAUGCUCCAGGCAGC |
| siRNA 337 | 337 | CUGCCUGGAGCAUGGAGCC | 3235 | GGCUCCAUGCUCCAGGCAG |
| siRNA 338 | 338 | UGCCUGGAGCAUGGAGCCG | 3236 | CGGCUCCAUGCUCCAGGCA |
| siRNA 339 | 339 | GCCUGGAGCAUGGAGCCGG | 3237 | CCGGCUCCAUGCUCCAGGC |
| siRNA 340 | 340 | CCUGGAGCAUGGAGCCGGU | 3238 | ACCGGCUCCAUGCUCCAGG |
| siRNA 341 | 341 | CUGGAGCAUGGAGCCGGUG | 3239 | CACCGGCUCCAUGCUCCAG |
| siRNA 342 | 342 | UGGAGCAUGGAGCCGGUGG | 3240 | CCACCGGCUCCAUGCUCCA |
| siRNA 343 | 343 | GGAGCAUGGAGCCGGUGGU | 3241 | ACCACCGGCUCCAUGCUCC |
| siRNA 344 | 344 | GAGCAUGGAGCCGGUGGUC | 3242 | GACCACCGGCUCCAUGCUC |
| siRNA 345 | 345 | AGCAUGGAGCCGGUGGUCC | 3243 | GGACCACCGGCUCCAUGCU |
| siRNA 346 | 346 | GCAUGGAGCCGGUGGUCCG | 3244 | CGGACCACCGGCUCCAUGC |
| siRNA 347 | 347 | CAUGGAGCCGGUGGUCCGC | 3245 | GCGGACCACCGGCUCCAUG |
| siRNA 348 | 348 | AUGGAGCCGGUGGUCCGCA | 3246 | UGCGGACCACCGGCUCCAU |
| siRNA 349 | 349 | UGGAGCCGGUGGUCCGCAG | 3247 | CUGCGGACCACCGGCUCCA |
| siRNA 350 | 350 | GGAGCCGGUGGUCCGCAGG | 3248 | CCUGCGGACCACCGGCUCC |
| siRNA 351 | 351 | GAGCCGGUGGUCCGCAGGC | 3249 | GCCUGCGGACCACCGGCUC |
| siRNA 352 | 352 | AGCCGGUGGUCCGCAGGCU | 3250 | AGCCUGCGGACCACCGGCU |
| siRNA 353 | 353 | GCCGGUGGUCCGCAGGCUG | 3251 | CAGCCUGCGGACCACCGGC |
| siRNA 354 | 354 | CCGGUGGUCCGCAGGCUGU | 3252 | ACAGCCUGCGGACCACCGG |
| siRNA 355 | 355 | CGGUGGUCCGCAGGCUGUC | 3253 | GACAGCCUGCGGACCACCG |
| siRNA 356 | 356 | GGUGGUCCGCAGGCUGUCC | 3254 | GGACAGCCUGCGGACCACC |
| siRNA 357 | 357 | GUGGUCCGCAGGCUGUCCA | 3255 | UGGACAGCCUGCGGACCAC |
| siRNA 358 | 358 | UGGUCCGCAGGCUGUCCAC | 3256 | GUGGACAGCCUGCGGACCA |
| siRNA 359 | 359 | GGUCCGCAGGCUGUCCACC | 3257 | GGUGGACAGCCUGCGGACC |
| siRNA 360 | 360 | GUCCGCAGGCUGUCCACCC | 3258 | GGGUGGACAGCCUGCGGAC |
| siRNA 361 | 361 | UCCGCAGGCUGUCCACCCA | 3259 | UGGGUGGACAGCCUGCGGA |
| siRNA 362 | 362 | CCGCAGGCUGUCCACCCAG | 3260 | CUGGGUGGACAGCCUGCGG |
| siRNA 363 | 363 | CGCAGGCUGUCCACCCAGU | 3261 | ACUGGGUGGACAGCCUGCG |
| siRNA 364 | 364 | GCAGGCUGUCCACCCAGUU | 3262 | AACUGGGUGGACAGCCUGC |
| siRNA 365 | 365 | CAGGCUGUCCACCCAGUUC | 3263 | GAACUGGGUGGACAGCCUG |
| siRNA 366 | 366 | AGGCUGUCCACCCAGUUCA | 3264 | UGAACUGGGUGGACAGCCU |
| siRNA 367 | 367 | GGCUGUCCACCCAGUUCAC | 3265 | GUGAACUGGGUGGACAGCC |
| siRNA 368 | 368 | GCUGUCCACCCAGUUCACA | 3266 | UGUGAACUGGGUGGACAGC |
| siRNA 369 | 369 | CUGUCCACCCAGUUCACAG | 3267 | CUGUGAACUGGGUGGACAG |
| siRNA 370 | 370 | UGUCCACCCAGUUCACAGC | 3268 | GCUGUGAACUGGGUGGACA |
| siRNA 371 | 371 | GUCCACCCAGUUCACAGCU | 3269 | AGCUGUGAACUGGGUGGAC |
| siRNA 372 | 372 | UCCACCCAGUUCACAGCUG | 3270 | CAGCUGUGAACUGGGUGGA |
| siRNA 373 | 373 | CCACCCAGUUCACAGCUGC | 3271 | GCAGCUGUGAACUGGGUGG |
| siRNA 374 | 374 | CACCCAGUUCACAGCUGCC | 3272 | GGCAGCUGUGAACUGGGUG |
| siRNA 375 | 375 | ACCCAGUUCACAGCUGCCA | 3273 | UGGCAGCUGUGAACUGGGU |
| siRNA 376 | 376 | CCCAGUUCACAGCUGCCAA | 3274 | UUGGCAGCUGUGAACUGGG |
| siRNA 377 | 377 | CCAGUUCACAGCUGCCAAU | 3275 | AUUGGCAGCUGUGAACUGG |
| siRNA 378 | 378 | CAGUUCACAGCUGCCAAUG | 3276 | CAUUGGCAGCUGUGAACUG |
| siRNA 379 | 379 | AGUUCACAGCUGCCAAUGA | 3277 | UCAUUGGCAGCUGUGAACU |
| siRNA 380 | 380 | GUUCACAGCUGCCAAUGAG | 3278 | CUCAUUGGCAGCUGUGAAC |
| siRNA 381 | 381 | UUCACAGCUGCCAAUGAGC | 3279 | GCUCAUUGGCAGCUGUGAA |
| siRNA 382 | 382 | UCACAGCUGCCAAUGAGCU | 3280 | AGCUCAUUGGCAGCUGUGA |
| siRNA 383 | 383 | CACAGCUGCCAAUGAGCUG | 3281 | CAGCUCAUUGGCAGCUGUG |
| siRNA 384 | 384 | ACAGCUGCCAAUGAGCUGG | 3282 | CCAGCUCAUUGGCAGCUGU |
| siRNA 385 | 385 | CAGCUGCCAAUGAGCUGGC | 3283 | GCCAGCUCAUUGGCAGCUG |
| siRNA 386 | 386 | AGCUGCCAAUGAGCUGGCC | 3284 | GGCCAGCUCAUUGGCAGCU |
| siRNA 387 | 387 | GCUGCCAAUGAGCUGGCCU | 3285 | AGGCCAGCUCAUUGGCAGC |
| siRNA 388 | 388 | CUGCCAAUGAGCUGGCCUG | 3286 | CAGGCCAGCUCAUUGGCAG |
| siRNA 389 | 389 | UGCCAAUGAGCUGGCCUGC | 3287 | GCAGGCCAGCUCAUUGGCA |
| siRNA 390 | 390 | GCCAAUGAGCUGGCCUGCC | 3288 | GGCAGGCCAGCUCAUUGGC |
| siRNA 391 | 391 | CCAAUGAGCUGGCCUGCCG | 3289 | CGGCAGGCCAGCUCAUUGG |
| siRNA 392 | 392 | CAAUGAGCUGGCCUGCCGA | 3290 | UCGGCAGGCCAGCUCAUUG |
| siRNA 393 | 393 | AAUGAGCUGGCCUGCCGAG | 3291 | CUCGGCAGGCCAGCUCAUU |
| siRNA 394 | 394 | AUGAGCUGGCCUGCCGAGG | 3292 | CCUCGGCAGGCCAGCUCAU |
| siRNA 395 | 395 | UGAGCUGGCCUGCCGAGGC | 3293 | GCCUCGGCAGGCCAGCUCA |
| siRNA 396 | 396 | GAGCUGGCCUGCCGAGGCU | 3294 | AGCCUCGGCAGGCCAGCUC |
| siRNA 397 | 397 | AGCUGGCCUGCCGAGGCUU | 3295 | AAGCCUCGGCAGGCCAGCU |
| siRNA 398 | 398 | GCUGGCCUGCCGAGGCUUG | 3296 | CAAGCCUCGGCAGGCCAGC |
| siRNA 399 | 399 | CUGGCCUGCCGAGGCUUGG | 3297 | CCAAGCCUCGGCAGGCCAG |
| siRNA 400 | 400 | UGGCCUGCCGAGGCUUGGA | 3298 | UCCAAGCCUCGGCAGGCCA |
| siRNA 401 | 401 | GGCCUGCCGAGGCUUGGAC | 3299 | GUCCAAGCCUCGGCAGGCC |
| siRNA 402 | 402 | GCCUGCCGAGGCUUGGACC | 3300 | GGUCCAAGCCUCGGCAGGC |
| siRNA 403 | 403 | CCUGCCGAGGCUUGGACCA | 3301 | UGGUCCAAGCCUCGGCAGG |
| siRNA 404 | 404 | CUGCCGAGGCUUGGACCAC | 3302 | GUGGUCCAAGCCUCGGCAG |
| siRNA 405 | 405 | UGCCGAGGCUUGGACCACC | 3303 | GGUGGUCCAAGCCUCGGCA |
| siRNA 406 | 406 | GCCGAGGCUUGGACCACCU | 3304 | AGGUGGUCCAAGCCUCGGC |
| siRNA 407 | 407 | CCGAGGCUUGGACCACCUG | 3305 | CAGGUGGUCCAAGCCUCGG |
| siRNA 408 | 408 | CGAGGCUUGGACCACCUGG | 3306 | CCAGGUGGUCCAAGCCUCG |
| siRNA 409 | 409 | GAGGCUUGGACCACCUGGA | 3307 | UCCAGGUGGUCCAAGCCUC |
| siRNA 410 | 410 | AGGCUUGGACCACCUGGAG | 3308 | CUCCAGGUGGUCCAAGCCU |
| siRNA 411 | 411 | GGCUUGGACCACCUGGAGG | 3309 | CCUCCAGGUGGUCCAAGCC |
| siRNA 412 | 412 | GCUUGGACCACCUGGAGGA | 3310 | UCCUCCAGGUGGUCCAAGC |
| siRNA 413 | 413 | CUUGGACCACCUGGAGGAA | 3311 | UUCCUCCAGGUGGUCCAAG |
| siRNA 414 | 414 | UUGGACCACCUGGAGGAAA | 3312 | UUUCCUCCAGGUGGUCCAA |
| siRNA 415 | 415 | UGGACCACCUGGAGGAAAA | 3313 | UUUUCCUCCAGGUGGUCCA |
| siRNA 416 | 416 | GGACCACCUGGAGGAAAAG | 3314 | CUUUUCCUCCAGGUGGUCC |
| siRNA 417 | 417 | GACCACCUGGAGGAAAAGA | 3315 | UCUUUUCCUCCAGGUGGUC |
| siRNA 418 | 418 | ACCACCUGGAGGAAAAGAU | 3316 | AUCUUUUCCUCCAGGUGGU |
| siRNA 419 | 419 | CCACCUGGAGGAAAAGAUC | 3317 | GAUCUUUUCCUCCAGGUGG |
| siRNA 420 | 420 | CACCUGGAGGAAAAGAUCC | 3318 | GGAUCUUUUCCUCCAGGUG |
| siRNA 421 | 421 | ACCUGGAGGAAAAGAUCCC | 3319 | GGGAUCUUUUCCUCCAGGU |
| siRNA 422 | 422 | CCUGGAGGAAAAGAUCCCC | 3320 | GGGGAUCUUUUCCUCCAGG |
| siRNA 423 | 423 | CUGGAGGAAAAGAUCCCCG | 3321 | CGGGGAUCUUUUCCUCCAG |
| siRNA 424 | 424 | UGGAGGAAAAGAUCCCCGC | 3322 | GCGGGGAUCUUUUCCUCCA |
| siRNA 425 | 425 | GGAGGAAAAGAUCCCCGCC | 3323 | GGCGGGGAUCUUUUCCUCC |
| siRNA 426 | 426 | GAGGAAAAGAUCCCCGCCC | 3324 | GGGCGGGGAUCUUUUCCUC |
| siRNA 427 | 427 | AGGAAAAGAUCCCCGCCCU | 3325 | AGGGGGGGGAUCUUUUCCU |
| siRNA 428 | 428 | GGAAAAGAUCCCCGCCCUC | 3326 | GAGGGGGGGGAUCUUUUCC |
| siRNA 429 | 429 | GAAAAGAUCCCCGCCCUCC | 3327 | GGAGGGCGGGGAUCUUUUC |
| siRNA 430 | 430 | AAAAGAUCCCCGCCCUCCA | 3328 | UGGAGGGGGGGGAUCUUUU |
| siRNA 431 | 431 | AAAGAUCCCCGCCCUCCAG | 3329 | CUGGAGGGGGGGGAUCUUU |
| siRNA 432 | 432 | AAGAUCCCCGCCCUCCAGU | 3330 | ACUGGAGGGGGGGGAUCUU |
| siRNA 433 | 433 | AGAUCCCCGCCCUCCAGUA | 3331 | UACUGGAGGGGGGGGAUCU |
| siRNA 434 | 434 | GAUCCCCGCCCUCCAGUAC | 3332 | GUACUGGAGGGGGGGGAUC |
| siRNA 435 | 435 | AUCCCCGCCCUCCAGUACC | 3333 | GGUACUGGAGGGCGGGGAU |
| siRNA 436 | 436 | UCCCCGCCCUCCAGUACCC | 3334 | GGGUACUGGAGGGGGGGGA |
| siRNA 437 | 437 | CCCCGCCCUCCAGUACCCC | 3335 | GGGGUACUGGAGGGCGGGG |
| siRNA 438 | 438 | CCCGCCCUCCAGUACCCCC | 3336 | GGGGGUACUGGAGGGCGGG |
| siRNA 439 | 439 | CCGCCCUCCAGUACCCCCC | 3337 | GGGGGGUACUGGAGGGCGG |
| siRNA 440 | 440 | CGCCCUCCAGUACCCCCCU | 3338 | AGGGGGGUACUGGAGGGCG |
| siRNA 441 | 441 | GCCCUCCAGUACCCCCCUG | 3339 | CAGGGGGGUACUGGAGGGC |
| siRNA 442 | 442 | CCCUCCAGUACCCCCCUGA | 3340 | UCAGGGGGGUACUGGAGGG |
| siRNA 443 | 443 | CCUCCAGUACCCCCCUGAA | 3341 | UUCAGGGGGGUACUGGAGG |
| siRNA 444 | 444 | CUCCAGUACCCCCCUGAAA | 3342 | UUUCAGGGGGGUACUGGAG |
| siRNA 445 | 445 | UCCAGUACCCCCCUGAAAA | 3343 | UUUUCAGGGGGGUACUGGA |
| siRNA 446 | 446 | CCAGUACCCCCCUGAAAAG | 3344 | CUUUUCAGGGGGGUACUGG |
| siRNA 447 | 447 | CAGUACCCCCCUGAAAAGA | 3345 | UCUUUUCAGGGGGGUACUG |
| siRNA 448 | 448 | AGUACCCCCCUGAAAAGAU | 3346 | AUCUUUUCAGGGGGGUACU |
| siRNA 449 | 449 | GUACCCCCCUGAAAAGAUU | 3347 | AAUCUUUUCAGGGGGGUAC |
| siRNA 450 | 450 | UACCCCCCUGAAAAGAUUG | 3348 | CAAUCUUUUCAGGGGGGUA |
| siRNA 451 | 451 | ACCCCCCUGAAAAGAUUGC | 3349 | GCAAUCUUUUCAGGGGGGU |
| siRNA 452 | 452 | CCCCCCUGAAAAGAUUGCU | 3350 | AGCAAUCUUUUCAGGGGGG |
| siRNA 453 | 453 | CCCCCUGAAAAGAUUGCUU | 3351 | AAGCAAUCUUUUCAGGGGG |
| siRNA 454 | 454 | CCCCUGAAAAGAUUGCUUC | 3352 | GAAGCAAUCUUUUCAGGGG |
| siRNA 455 | 455 | CCCUGAAAAGAUUGCUUCU | 3353 | AGAAGCAAUCUUUUCAGGG |
| siRNA 456 | 456 | CCUGAAAAGAUUGCUUCUG | 3354 | CAGAAGCAAUCUUUUCAGG |
| siRNA 457 | 457 | CUGAAAAGAUUGCUUCUGA | 3355 | UCAGAAGCAAUCUUUUCAG |
| siRNA 458 | 458 | UGAAAAGAUUGCUUCUGAG | 3356 | CUCAGAAGCAAUCUUUUCA |
| siRNA 459 | 459 | GAAAAGAUUGCUUCUGAGC | 3357 | GCUCAGAAGCAAUCUUUUC |
| siRNA 460 | 460 | AAAAGAUUGCUUCUGAGCU | 3358 | AGCUCAGAAGCAAUCUUUU |
| siRNA 461 | 461 | AAAGAUUGCUUCUGAGCUG | 3359 | CAGCUCAGAAGCAAUCUUU |
| siRNA 462 | 462 | AAGAUUGCUUCUGAGCUGA | 3360 | UCAGCUCAGAAGCAAUCUU |
| siRNA 463 | 463 | AGAUUGCUUCUGAGCUGAA | 3361 | UUCAGCUCAGAAGCAAUCU |
| siRNA 464 | 464 | GAUUGCUUCUGAGCUGAAG | 3362 | CUUCAGCUCAGAAGCAAUC |
| siRNA 465 | 465 | AUUGCUUCUGAGCUGAAGG | 3363 | CCUUCAGCUCAGAAGCAAU |
| siRNA 466 | 466 | UUGCUUCUGAGCUGAAGGA | 3364 | UCCUUCAGCUCAGAAGCAA |
| siRNA 467 | 467 | UGCUUCUGAGCUGAAGGAC | 3365 | GUCCUUCAGCUCAGAAGCA |
| siRNA 468 | 468 | GCUUCUGAGCUGAAGGACA | 3366 | UGUCCUUCAGCUCAGAAGC |
| siRNA 469 | 469 | CUUCUGAGCUGAAGGACAC | 3367 | GUGUCCUUCAGCUCAGAAG |
| siRNA 470 | 470 | UUCUGAGCUGAAGGACACC | 3368 | GGUGUCCUUCAGCUCAGAA |
| siRNA 471 | 471 | UCUGAGCUGAAGGACACCA | 3369 | UGGUGUCCUUCAGCUCAGA |
| siRNA 472 | 472 | CUGAGCUGAAGGACACCAU | 3370 | AUGGUGUCCUUCAGCUCAG |
| siRNA 473 | 473 | UGAGCUGAAGGACACCAUC | 3371 | GAUGGUGUCCUUCAGCUCA |
| siRNA 474 | 474 | GAGCUGAAGGACACCAUCU | 3372 | AGAUGGUGUCCUUCAGCUC |
| siRNA 475 | 475 | AGCUGAAGGACACCAUCUC | 3373 | GAGAUGGUGUCCUUCAGCU |
| siRNA 476 | 476 | GCUGAAGGACACCAUCUCC | 3374 | GGAGAUGGUGUCCUUCAGC |
| siRNA 477 | 477 | CUGAAGGACACCAUCUCCA | 3375 | UGGAGAUGGUGUCCUUCAG |
| siRNA 478 | 478 | UGAAGGACACCAUCUCCAC | 3376 | GUGGAGAUGGUGUCCUUCA |
| siRNA 479 | 479 | GAAGGACACCAUCUCCACC | 3377 | GGUGGAGAUGGUGUCCUUC |
| siRNA 480 | 480 | AAGGACACCAUCUCCACCC | 3378 | GGGUGGAGAUGGUGUCCUU |
| siRNA 481 | 481 | AGGACACCAUCUCCACCCG | 3379 | CGGGUGGAGAUGGUGUCCU |
| siRNA 482 | 482 | GGACACCAUCUCCACCCGC | 3380 | GCGGGUGGAGAUGGUGUCC |
| siRNA 483 | 483 | GACACCAUCUCCACCCGCC | 3381 | GGCGGGUGGAGAUGGUGUC |
| siRNA 484 | 484 | ACACCAUCUCCACCCGCCU | 3382 | AGGCGGGUGGAGAUGGUGU |
| siRNA 485 | 485 | CACCAUCUCCACCCGCCUC | 3383 | GAGGCGGGUGGAGAUGGUG |
| siRNA 486 | 486 | ACCAUCUCCACCCGCCUCC | 3384 | GGAGGCGGGUGGAGAUGGU |
| siRNA 487 | 487 | CCAUCUCCACCCGCCUCCG | 3385 | CGGAGGCGGGUGGAGAUGG |
| siRNA 488 | 488 | CAUCUCCACCCGCCUCCGC | 3386 | GCGGAGGCGGGUGGAGAUG |
| siRNA 489 | 489 | AUCUCCACCCGCCUCCGCA | 3387 | UGCGGAGGCGGGUGGAGAU |
| siRNA 490 | 490 | UCUCCACCCGCCUCCGCAG | 3388 | CUGCGGAGGCGGGUGGAGA |
| siRNA 491 | 491 | CUCCACCCGCCUCCGCAGU | 3389 | ACUGCGGAGGCGGGUGGAG |
| siRNA 492 | 492 | UCCACCCGCCUCCGCAGUG | 3390 | CACUGCGGAGGCGGGUGGA |
| siRNA 493 | 493 | CCACCCGCCUCCGCAGUGC | 3391 | GCACUGCGGAGGCGGGUGG |
| siRNA 494 | 494 | CACCCGCCUCCGCAGUGCC | 3392 | GGCACUGCGGAGGCGGGUG |
| siRNA 495 | 495 | ACCCGCCUCCGCAGUGCCA | 3393 | UGGCACUGCGGAGGCGGGU |
| siRNA 496 | 496 | CCCGCCUCCGCAGUGCCAG | 3394 | CUGGCACUGCGGAGGCGGG |
| siRNA 497 | 497 | CCGCCUCCGCAGUGCCAGA | 3395 | UCUGGCACUGCGGAGGCGG |
| siRNA 498 | 498 | CGCCUCCGCAGUGCCAGAA | 3396 | UUCUGGCACUGCGGAGGCG |
| siRNA 499 | 499 | GCCUCCGCAGUGCCAGAAA | 3397 | UUUCUGGCACUGCGGAGGC |
| siRNA 500 | 500 | CCUCCGCAGUGCCAGAAAC | 3398 | GUUUCUGGCACUGCGGAGG |
| siRNA 501 | 501 | CUCCGCAGUGCCAGAAACA | 3399 | UGUUUCUGGCACUGCGGAG |
| siRNA 502 | 502 | UCCGCAGUGCCAGAAACAG | 3400 | CUGUUUCUGGCACUGCGGA |
| siRNA 503 | 503 | CCGCAGUGCCAGAAACAGC | 3401 | GCUGUUUCUGGCACUGCGG |
| siRNA 504 | 504 | CGCAGUGCCAGAAACAGCA | 3402 | UGCUGUUUCUGGCACUGCG |
| siRNA 505 | 505 | GCAGUGCCAGAAACAGCAU | 3403 | AUGCUGUUUCUGGCACUGC |
| siRNA 506 | 506 | CAGUGCCAGAAACAGCAUC | 3404 | GAUGCUGUUUCUGGCACUG |
| siRNA 507 | 507 | AGUGCCAGAAACAGCAUCA | 3405 | UGAUGCUGUUUCUGGCACU |
| siRNA 508 | 508 | GUGCCAGAAACAGCAUCAG | 3406 | CUGAUGCUGUUUCUGGCAC |
| siRNA 509 | 509 | UGCCAGAAACAGCAUCAGC | 3407 | GCUGAUGCUGUUUCUGGCA |
| siRNA 510 | 510 | GCCAGAAACAGCAUCAGCG | 3408 | CGCUGAUGCUGUUUCUGGC |
| siRNA 511 | 511 | CCAGAAACAGCAUCAGCGU | 3409 | ACGCUGAUGCUGUUUCUGG |
| siRNA 512 | 512 | CAGAAACAGCAUCAGCGUU | 3410 | AACGCUGAUGCUGUUUCUG |
| siRNA 513 | 513 | AGAAACAGCAUCAGCGUUC | 3411 | GAACGCUGAUGCUGUUUCU |
| siRNA 514 | 514 | GAAACAGCAUCAGCGUUCC | 3412 | GGAACGCUGAUGCUGUUUC |
| siRNA 515 | 515 | AAACAGCAUCAGCGUUCCC | 3413 | GGGAACGCUGAUGCUGUUU |
| siRNA 516 | 516 | AACAGCAUCAGCGUUCCCA | 3414 | UGGGAACGCUGAUGCUGUU |
| siRNA 517 | 517 | ACAGCAUCAGCGUUCCCAU | 3415 | AUGGGAACGCUGAUGCUGU |
| siRNA 518 | 518 | CAGCAUCAGCGUUCCCAUC | 3416 | GAUGGGAACGCUGAUGCUG |
| siRNA 519 | 519 | AGCAUCAGCGUUCCCAUCG | 3417 | CGAUGGGAACGCUGAUGCU |
| siRNA 520 | 520 | GCAUCAGCGUUCCCAUCGC | 3418 | GCGAUGGGAACGCUGAUGC |
| siRNA 521 | 521 | CAUCAGCGUUCCCAUCGCG | 3419 | CGCGAUGGGAACGCUGAUG |
| siRNA 522 | 522 | AUCAGCGUUCCCAUCGCGA | 3420 | UCGCGAUGGGAACGCUGAU |
| siRNA 523 | 523 | UCAGCGUUCCCAUCGCGAG | 3421 | CUCGCGAUGGGAACGCUGA |
| siRNA 524 | 524 | CAGCGUUCCCAUCGCGAGC | 3422 | GCUCGCGAUGGGAACGCUG |
| siRNA 525 | 525 | AGCGUUCCCAUCGCGAGCA | 3423 | UGCUCGCGAUGGGAACGCU |
| siRNA 526 | 526 | GCGUUCCCAUCGCGAGCAC | 3424 | GUGCUCGCGAUGGGAACGC |
| siRNA 527 | 527 | CGUUCCCAUCGCGAGCACU | 3425 | AGUGCUCGCGAUGGGAACG |
| siRNA 528 | 528 | GUUCCCAUCGCGAGCACUU | 3426 | AAGUGCUCGCGAUGGGAAC |
| siRNA 529 | 529 | UUCCCAUCGCGAGCACUUC | 3427 | GAAGUGCUCGCGAUGGGAA |
| siRNA 530 | 530 | UCCCAUCGCGAGCACUUCA | 3428 | UGAAGUGCUCGCGAUGGGA |
| siRNA 531 | 531 | CCCAUCGCGAGCACUUCAG | 3429 | CUGAAGUGCUCGCGAUGGG |
| siRNA 532 | 532 | CCAUCGCGAGCACUUCAGA | 3430 | UCUGAAGUGCUCGCGAUGG |
| siRNA 533 | 533 | CAUCGCGAGCACUUCAGAC | 3431 | GUCUGAAGUGCUCGCGAUG |
| siRNA 534 | 534 | AUCGCGAGCACUUCAGACA | 3432 | UGUCUGAAGUGCUCGCGAU |
| siRNA 535 | 535 | UCGCGAGCACUUCAGACAA | 3433 | UUGUCUGAAGUGCUCGCGA |
| siRNA 536 | 536 | CGCGAGCACUUCAGACAAG | 3434 | CUUGUCUGAAGUGCUCGCG |
| siRNA 537 | 537 | GCGAGCACUUCAGACAAGG | 3435 | CCUUGUCUGAAGUGCUCGC |
| siRNA 538 | 538 | CGAGCACUUCAGACAAGGU | 3436 | ACCUUGUCUGAAGUGCUCG |
| siRNA 539 | 539 | GAGCACUUCAGACAAGGUC | 3437 | GACCUUGUCUGAAGUGCUC |
| siRNA 540 | 540 | AGCACUUCAGACAAGGUCC | 3438 | GGACCUUGUCUGAAGUGCU |
| siRNA 541 | 541 | GCACUUCAGACAAGGUCCU | 3439 | AGGACCUUGUCUGAAGUGC |
| siRNA 542 | 542 | CACUUCAGACAAGGUCCUG | 3440 | CAGGACCUUGUCUGAAGUG |
| siRNA 543 | 543 | ACUUCAGACAAGGUCCUGG | 3441 | CCAGGACCUUGUCUGAAGU |
| siRNA 544 | 544 | CUUCAGACAAGGUCCUGGG | 3442 | CCCAGGACCUUGUCUGAAG |
| siRNA 545 | 545 | UUCAGACAAGGUCCUGGGG | 3443 | CCCCAGGACCUUGUCUGAA |
| siRNA 546 | 546 | UCAGACAAGGUCCUGGGGG | 3444 | CCCCCAGGACCUUGUCUGA |
| siRNA 547 | 547 | CAGACAAGGUCCUGGGGGC | 3445 | GCCCCCAGGACCUUGUCUG |
| siRNA 548 | 548 | AGACAAGGUCCUGGGGGCC | 3446 | GGCCCCCAGGACCUUGUCU |
| siRNA 549 | 549 | GACAAGGUCCUGGGGGCCG | 3447 | CGGCCCCCAGGACCUUGUC |
| siRNA 550 | 550 | ACAAGGUCCUGGGGGCCGC | 3448 | GCGGCCCCCAGGACCUUGU |
| siRNA 551 | 551 | CAAGGUCCUGGGGGCCGCU | 3449 | AGCGGCCCCCAGGACCUUG |
| siRNA 552 | 552 | AAGGUCCUGGGGGCCGCUU | 3450 | AAGCGGCCCCCAGGACCUU |
| siRNA 553 | 553 | AGGUCCUGGGGGCCGCUUU | 3451 | AAAGCGGCCCCCAGGACCU |
| siRNA 554 | 554 | GGUCCUGGGGGCCGCUUUG | 3452 | CAAAGCGGCCCCCAGGACC |
| siRNA 555 | 555 | GUCCUGGGGGCCGCUUUGG | 3453 | CCAAAGCGGCCCCCAGGAC |
| siRNA 556 | 556 | UCCUGGGGGCCGCUUUGGC | 3454 | GCCAAAGCGGCCCCCAGGA |
| siRNA 557 | 557 | CCUGGGGGCCGCUUUGGCC | 3455 | GGCCAAAGCGGCCCCCAGG |
| siRNA 558 | 558 | CUGGGGGCCGCUUUGGCCG | 3456 | CGGCCAAAGCGGCCCCCAG |
| siRNA 559 | 559 | UGGGGGCCGCUUUGGCCGG | 3457 | CCGGCCAAAGCGGCCCCCA |
| siRNA 560 | 560 | GGGGGCCGCUUUGGCCGGG | 3458 | CCCGGCCAAAGCGGCCCCC |
| siRNA 561 | 561 | GGGGCCGCUUUGGCCGGGU | 3459 | ACCCGGCCAAAGCGGCCCC |
| siRNA 562 | 562 | GGGCCGCUUUGGCCGGGUG | 3460 | CACCCGGCCAAAGCGGCCC |
| siRNA 563 | 563 | GGCCGCUUUGGCCGGGUGC | 3461 | GCACCCGGCCAAAGCGGCC |
| siRNA 564 | 564 | GCCGCUUUGGCCGGGUGCG | 3462 | CGCACCCGGCCAAAGCGGC |
| siRNA 565 | 565 | CCGCUUUGGCCGGGUGCGA | 3463 | UCGCACCCGGCCAAAGCGG |
| siRNA 566 | 566 | CGCUUUGGCCGGGUGCGAG | 3464 | CUCGCACCCGGCCAAAGCG |
| siRNA 567 | 567 | GCUUUGGCCGGGUGCGAGC | 3465 | GCUCGCACCCGGCCAAAGC |
| siRNA 568 | 568 | CUUUGGCCGGGUGCGAGCU | 3466 | AGCUCGCACCCGGCCAAAG |
| siRNA 569 | 569 | UUUGGCCGGGUGCGAGCUU | 3467 | AAGCUCGCACCCGGCCAAA |
| siRNA 570 | 570 | UUGGCCGGGUGCGAGCUUG | 3468 | CAAGCUCGCACCCGGCCAA |
| siRNA 571 | 571 | UGGCCGGGUGCGAGCUUGC | 3469 | GCAAGCUCGCACCCGGCCA |
| siRNA 572 | 572 | GGCCGGGUGCGAGCUUGCC | 3470 | GGCAAGCUCGCACCCGGCC |
| siRNA 573 | 573 | GCCGGGUGCGAGCUUGCCU | 3471 | AGGCAAGCUCGCACCCGGC |
| siRNA 574 | 574 | CCGGGUGCGAGCUUGCCUG | 3472 | CAGGCAAGCUCGCACCCGG |
| siRNA 575 | 575 | CGGGUGCGAGCUUGCCUGG | 3473 | CCAGGCAAGCUCGCACCCG |
| siRNA 576 | 576 | GGGUGCGAGCUUGCCUGGG | 3474 | CCCAGGCAAGCUCGCACCC |
| siRNA 577 | 577 | GGUGCGAGCUUGCCUGGGG | 3475 | CCCCAGGCAAGCUCGCACC |
| siRNA 578 | 578 | GUGCGAGCUUGCCUGGGGG | 3476 | CCCCCAGGCAAGCUCGCAC |
| siRNA 579 | 579 | UGCGAGCUUGCCUGGGGGG | 3477 | CCCCCCAGGCAAGCUCGCA |
| SIRNA 580 | 580 | GCGAGCUUGCCUGGGGGGU | 3478 | ACCCCCCAGGCAAGCUCGC |
| siRNA 581 | 581 | CGAGCUUGCCUGGGGGGUG | 3479 | CACCCCCCAGGCAAGCUCG |
| siRNA 582 | 582 | GAGCUUGCCUGGGGGGUGG | 3480 | CCACCCCCCAGGCAAGCUC |
| siRNA 583 | 583 | AGCUUGCCUGGGGGGUGGC | 3481 | GCCACCCCCCAGGCAAGCU |
| siRNA 584 | 584 | GCUUGCCUGGGGGGUGGCC | 3482 | GGCCACCCCCCAGGCAAGC |
| siRNA 585 | 585 | CUUGCCUGGGGGGUGGCCA | 3483 | UGGCCACCCCCCAGGCAAG |
| siRNA 586 | 586 | UUGCCUGGGGGGUGGCCAG | 3484 | CUGGCCACCCCCCAGGCAA |
| siRNA 587 | 587 | UGCCUGGGGGGUGGCCAGA | 3485 | UCUGGCCACCCCCCAGGCA |
| siRNA 588 | 588 | GCCUGGGGGGUGGCCAGAG | 3486 | CUCUGGCCACCCCCCAGGC |
| siRNA 589 | 589 | CCUGGGGGGUGGCCAGAGA | 3487 | UCUCUGGCCACCCCCCAGG |
| siRNA 590 | 590 | CUGGGGGGUGGCCAGAGAC | 3488 | GUCUCUGGCCACCCCCCAG |
| siRNA 591 | 591 | UGGGGGGUGGCCAGAGACA | 3489 | UGUCUCUGGCCACCCCCCA |
| siRNA 592 | 592 | GGGGGGUGGCCAGAGACAC | 3490 | GUGUCUCUGGCCACCCCCC |
| siRNA 593 | 593 | GGGGGUGGCCAGAGACACU | 3491 | AGUGUCUCUGGCCACCCCC |
| siRNA 594 | 594 | GGGGUGGCCAGAGACACUG | 3492 | CAGUGUCUCUGGCCACCCC |
| siRNA 595 | 595 | GGGUGGCCAGAGACACUGC | 3493 | GCAGUGUCUCUGGCCACCC |
| siRNA 596 | 596 | GGUGGCCAGAGACACUGCG | 3494 | CGCAGUGUCUCUGGCCACC |
| siRNA 597 | 597 | GUGGCCAGAGACACUGCGG | 3495 | CCGCAGUGUCUCUGGCCAC |
| siRNA 598 | 598 | UGGCCAGAGACACUGCGGA | 3496 | UCCGCAGUGUCUCUGGCCA |
| siRNA 599 | 599 | GGCCAGAGACACUGCGGAA | 3497 | UUCCGCAGUGUCUCUGGCC |
| siRNA 600 | 600 | GCCAGAGACACUGCGGAAU | 3498 | AUUCCGCAGUGUCUCUGGC |
| siRNA 601 | 601 | CCAGAGACACUGCGGAAUU | 3499 | AAUUCCGCAGUGUCUCUGG |
| siRNA 602 | 602 | CAGAGACACUGCGGAAUUU | 3500 | AAAUUCCGCAGUGUCUCUG |
| siRNA 603 | 603 | AGAGACACUGCGGAAUUUG | 3501 | CAAAUUCCGCAGUGUCUCU |
| siRNA 604 | 604 | GAGACACUGCGGAAUUUGC | 3502 | GCAAAUUCCGCAGUGUCUC |
| siRNA 605 | 605 | AGACACUGCGGAAUUUGCU | 3503 | AGCAAAUUCCGCAGUGUCU |
| siRNA 606 | 606 | GACACUGCGGAAUUUGCUG | 3504 | CAGCAAAUUCCGCAGUGUC |
| siRNA 607 | 607 | ACACUGCGGAAUUUGCUGC | 3505 | GCAGCAAAUUCCGCAGUGU |
| siRNA 608 | 608 | CACUGCGGAAUUUGCUGCC | 3506 | GGCAGCAAAUUCCGCAGUG |
| siRNA 609 | 609 | ACUGCGGAAUUUGCUGCCA | 3507 | UGGCAGCAAAUUCCGCAGU |
| siRNA 610 | 610 | CUGCGGAAUUUGCUGCCAA | 3508 | UUGGCAGCAAAUUCCGCAG |
| siRNA 611 | 611 | UGCGGAAUUUGCUGCCAAC | 3509 | GUUGGCAGCAAAUUCCGCA |
| siRNA 612 | 612 | GCGGAAUUUGCUGCCAACA | 3510 | UGUUGGCAGCAAAUUCCGC |
| siRNA 613 | 613 | CGGAAUUUGCUGCCAACAC | 3511 | GUGUUGGCAGCAAAUUCCG |
| siRNA 614 | 614 | GGAAUUUGCUGCCAACACU | 3512 | AGUGUUGGCAGCAAAUUCC |
| siRNA 615 | 615 | GAAUUUGCUGCCAACACUC | 3513 | GAGUGUUGGCAGCAAAUUC |
| siRNA 616 | 616 | AAUUUGCUGCCAACACUCG | 3514 | CGAGUGUUGGCAGCAAAUU |
| siRNA 617 | 617 | AUUUGCUGCCAACACUCGA | 3515 | UCGAGUGUUGGCAGCAAAU |
| siRNA 618 | 618 | UUUGCUGCCAACACUCGAG | 3516 | CUCGAGUGUUGGCAGCAAA |
| siRNA 619 | 619 | UUGCUGCCAACACUCGAGC | 3517 | GCUCGAGUGUUGGCAGCAA |
| siRNA 620 | 620 | UGCUGCCAACACUCGAGCU | 3518 | AGCUCGAGUGUUGGCAGCA |
| siRNA 621 | 621 | GCUGCCAACACUCGAGCUG | 3519 | CAGCUCGAGUGUUGGCAGC |
| siRNA 622 | 622 | CUGCCAACACUCGAGCUGG | 3520 | CCAGCUCGAGUGUUGGCAG |
| siRNA 623 | 623 | UGCCAACACUCGAGCUGGC | 3521 | GCCAGCUCGAGUGUUGGCA |
| siRNA 624 | 624 | GCCAACACUCGAGCUGGCC | 3522 | GGCCAGCUCGAGUGUUGGC |
| siRNA 625 | 625 | CCAACACUCGAGCUGGCCG | 3523 | CGGCCAGCUCGAGUGUUGG |
| siRNA 626 | 626 | CAACACUCGAGCUGGCCGA | 3524 | UCGGCCAGCUCGAGUGUUG |
| siRNA 627 | 627 | AACACUCGAGCUGGCCGAC | 3525 | GUCGGCCAGCUCGAGUGUU |
| siRNA 628 | 628 | ACACUCGAGCUGGCCGACU | 3526 | AGUCGGCCAGCUCGAGUGU |
| siRNA 629 | 629 | CACUCGAGCUGGCCGACUG | 3527 | CAGUCGGCCAGCUCGAGUG |
| siRNA 630 | 630 | ACUCGAGCUGGCCGACUGG | 3528 | CCAGUCGGCCAGCUCGAGU |
| siRNA 631 | 631 | CUCGAGCUGGCCGACUGGC | 3529 | GCCAGUCGGCCAGCUCGAG |
| siRNA 632 | 632 | UCGAGCUGGCCGACUGGCU | 3530 | AGCCAGUCGGCCAGCUCGA |
| siRNA 633 | 633 | CGAGCUGGCCGACUGGCUU | 3531 | AAGCCAGUCGGCCAGCUCG |
| siRNA 634 | 634 | GAGCUGGCCGACUGGCUUC | 3532 | GAAGCCAGUCGGCCAGCUC |
| siRNA 635 | 635 | AGCUGGCCGACUGGCUUCU | 3533 | AGAAGCCAGUCGGCCAGCU |
| siRNA 636 | 636 | GCUGGCCGACUGGCUUCUG | 3534 | CAGAAGCCAGUCGGCCAGC |
| siRNA 637 | 637 | CUGGCCGACUGGCUUCUGG | 3535 | CCAGAAGCCAGUCGGCCAG |
| siRNA 638 | 638 | UGGCCGACUGGCUUCUGGA | 3536 | UCCAGAAGCCAGUCGGCCA |
| SIRNA 639 | 639 | GGCCGACUGGCUUCUGGAG | 3537 | CUCCAGAAGCCAGUCGGCC |
| siRNA 640 | 640 | GCCGACUGGCUUCUGGAGG | 3538 | CCUCCAGAAGCCAGUCGGC |
| siRNA 641 | 641 | CCGACUGGCUUCUGGAGGG | 3539 | CCCUCCAGAAGCCAGUCGG |
| siRNA 642 | 642 | CGACUGGCUUCUGGAGGGG | 3540 | CCCCUCCAGAAGCCAGUCG |
| siRNA 643 | 643 | GACUGGCUUCUGGAGGGGC | 3541 | GCCCCUCCAGAAGCCAGUC |
| siRNA 644 | 644 | ACUGGCUUCUGGAGGGGCC | 3542 | GGCCCCUCCAGAAGCCAGU |
| siRNA 645 | 645 | CUGGCUUCUGGAGGGGCCG | 3543 | CGGCCCCUCCAGAAGCCAG |
| siRNA 646 | 646 | UGGCUUCUGGAGGGGCCGA | 3544 | UCGGCCCCUCCAGAAGCCA |
| siRNA 647 | 647 | GGCUUCUGGAGGGGCCGAC | 3545 | GUCGGCCCCUCCAGAAGCC |
| siRNA 648 | 648 | GCUUCUGGAGGGGCCGACU | 3546 | AGUCGGCCCCUCCAGAAGC |
| siRNA 649 | 649 | CUUCUGGAGGGGCCGACUU | 3547 | AAGUCGGCCCCUCCAGAAG |
| siRNA 650 | 650 | UUCUGGAGGGGCCGACUUG | 3548 | CAAGUCGGCCCCUCCAGAA |
| siRNA 651 | 651 | UCUGGAGGGGCCGACUUGG | 3549 | CCAAGUCGGCCCCUCCAGA |
| siRNA 652 | 652 | CUGGAGGGGCCGACUUGGC | 3550 | GCCAAGUCGGCCCCUCCAG |
| siRNA 653 | 653 | UGGAGGGGCCGACUUGGCC | 3551 | GGCCAAGUCGGCCCCUCCA |
| siRNA 654 | 654 | GGAGGGGCCGACUUGGCCU | 3552 | AGGCCAAGUCGGCCCCUCC |
| siRNA 655 | 655 | GAGGGGCCGACUUGGCCUU | 3553 | AAGGCCAAGUCGGCCCCUC |
| siRNA 656 | 656 | AGGGGCCGACUUGGCCUUG | 3554 | CAAGGCCAAGUCGGCCCCU |
| siRNA 657 | 657 | GGGGCCGACUUGGCCUUGG | 3555 | CCAAGGCCAAGUCGGCCCC |
| siRNA 658 | 658 | GGGCCGACUUGGCCUUGGG | 3556 | CCCAAGGCCAAGUCGGCCC |
| siRNA 659 | 659 | GGCCGACUUGGCCUUGGGC | 3557 | GCCCAAGGCCAAGUCGGCC |
| siRNA 660 | 660 | GCCGACUUGGCCUUGGGCA | 3558 | UGCCCAAGGCCAAGUCGGC |
| siRNA 661 | 661 | CCGACUUGGCCUUGGGCAG | 3559 | CUGCCCAAGGCCAAGUCGG |
| siRNA 662 | 662 | CGACUUGGCCUUGGGCAGC | 3560 | GCUGCCCAAGGCCAAGUCG |
| siRNA 663 | 663 | GACUUGGCCUUGGGCAGCA | 3561 | UGCUGCCCAAGGCCAAGUC |
| siRNA 664 | 664 | ACUUGGCCUUGGGCAGCAU | 3562 | AUGCUGCCCAAGGCCAAGU |
| siRNA 665 | 665 | CUUGGCCUUGGGCAGCAUU | 3563 | AAUGCUGCCCAAGGCCAAG |
| siRNA 666 | 666 | UUGGCCUUGGGCAGCAUUG | 3564 | CAAUGCUGCCCAAGGCCAA |
| siRNA 667 | 667 | UGGCCUUGGGCAGCAUUGA | 3565 | UCAAUGCUGCCCAAGGCCA |
| siRNA 668 | 668 | GGCCUUGGGCAGCAUUGAG | 3566 | CUCAAUGCUGCCCAAGGCC |
| siRNA 669 | 669 | GCCUUGGGCAGCAUUGAGA | 3567 | UCUCAAUGCUGCCCAAGGC |
| siRNA 670 | 670 | CCUUGGGCAGCAUUGAGAA | 3568 | UUCUCAAUGCUGCCCAAGG |
| siRNA 671 | 671 | CUUGGGCAGCAUUGAGAAG | 3569 | CUUCUCAAUGCUGCCCAAG |
| siRNA 672 | 672 | UUGGGCAGCAUUGAGAAGG | 3570 | CCUUCUCAAUGCUGCCCAA |
| siRNA 673 | 673 | UGGGCAGCAUUGAGAAGGU | 3571 | ACCUUCUCAAUGCUGCCCA |
| siRNA 674 | 674 | GGGCAGCAUUGAGAAGGUG | 3572 | CACCUUCUCAAUGCUGCCC |
| siRNA 675 | 675 | GGCAGCAUUGAGAAGGUGG | 3573 | CCACCUUCUCAAUGCUGCC |
| siRNA 676 | 676 | GCAGCAUUGAGAAGGUGGU | 3574 | ACCACCUUCUCAAUGCUGC |
| siRNA 677 | 677 | CAGCAUUGAGAAGGUGGUG | 3575 | CACCACCUUCUCAAUGCUG |
| siRNA 678 | 678 | AGCAUUGAGAAGGUGGUGG | 3576 | CCACCACCUUCUCAAUGCU |
| siRNA 679 | 679 | GCAUUGAGAAGGUGGUGGA | 3577 | UCCACCACCUUCUCAAUGC |
| siRNA 680 | 680 | CAUUGAGAAGGUGGUGGAG | 3578 | CUCCACCACCUUCUCAAUG |
| siRNA 681 | 681 | AUUGAGAAGGUGGUGGAGU | 3579 | ACUCCACCACCUUCUCAAU |
| siRNA 682 | 682 | UUGAGAAGGUGGUGGAGUA | 3580 | UACUCCACCACCUUCUCAA |
| siRNA 683 | 683 | UGAGAAGGUGGUGGAGUAC | 3581 | GUACUCCACCACCUUCUCA |
| siRNA 684 | 684 | GAGAAGGUGGUGGAGUACC | 3582 | GGUACUCCACCACCUUCUC |
| siRNA 685 | 685 | AGAAGGUGGUGGAGUACCU | 3583 | AGGUACUCCACCACCUUCU |
| siRNA 686 | 686 | GAAGGUGGUGGAGUACCUC | 3584 | GAGGUACUCCACCACCUUC |
| siRNA 687 | 687 | AAGGUGGUGGAGUACCUCC | 3585 | GGAGGUACUCCACCACCUU |
| siRNA 688 | 688 | AGGUGGUGGAGUACCUCCU | 3586 | AGGAGGUACUCCACCACCU |
| siRNA 689 | 689 | GGUGGUGGAGUACCUCCUC | 3587 | GAGGAGGUACUCCACCACC |
| siRNA 690 | 690 | GUGGUGGAGUACCUCCUCC | 3588 | GGAGGAGGUACUCCACCAC |
| siRNA 691 | 691 | UGGUGGAGUACCUCCUCCC | 3589 | GGGAGGAGGUACUCCACCA |
| siRNA 692 | 692 | GGUGGAGUACCUCCUCCCU | 3590 | AGGGAGGAGGUACUCCACC |
| siRNA 693 | 693 | GUGGAGUACCUCCUCCCUC | 3591 | GAGGGAGGAGGUACUCCAC |
| siRNA 694 | 694 | UGGAGUACCUCCUCCCUCC | 3592 | GGAGGGAGGAGGUACUCCA |
| siRNA 695 | 695 | GGAGUACCUCCUCCCUCCA | 3593 | UGGAGGGAGGAGGUACUCC |
| siRNA 696 | 696 | GAGUACCUCCUCCCUCCAG | 3594 | CUGGAGGGAGGAGGUACUC |
| siRNA 697 | 697 | AGUACCUCCUCCCUCCAGA | 3595 | UCUGGAGGGAGGAGGUACU |
| siRNA 698 | 698 | GUACCUCCUCCCUCCAGAC | 3596 | GUCUGGAGGGAGGAGGUAC |
| siRNA 699 | 699 | UACCUCCUCCCUCCAGACA | 3597 | UGUCUGGAGGGAGGAGGUA |
| siRNA 700 | 700 | ACCUCCUCCCUCCAGACAA | 3598 | UUGUCUGGAGGGAGGAGGU |
| siRNA 701 | 701 | CCUCCUCCCUCCAGACAAG | 3599 | CUUGUCUGGAGGGAGGAGG |
| siRNA 702 | 702 | CUCCUCCCUCCAGACAAGG | 3600 | CCUUGUCUGGAGGGAGGAG |
| siRNA 703 | 703 | UCCUCCCUCCAGACAAGGA | 3601 | UCCUUGUCUGGAGGGAGGA |
| siRNA 704 | 704 | CCUCCCUCCAGACAAGGAA | 3602 | UUCCUUGUCUGGAGGGAGG |
| siRNA 705 | 705 | CUCCCUCCAGACAAGGAAG | 3603 | CUUCCUUGUCUGGAGGGAG |
| siRNA 706 | 706 | UCCCUCCAGACAAGGAAGA | 3604 | UCUUCCUUGUCUGGAGGGA |
| siRNA 707 | 707 | CCCUCCAGACAAGGAAGAG | 3605 | CUCUUCCUUGUCUGGAGGG |
| siRNA 708 | 708 | CCUCCAGACAAGGAAGAGU | 3606 | ACUCUUCCUUGUCUGGAGG |
| siRNA 709 | 709 | CUCCAGACAAGGAAGAGUC | 3607 | GACUCUUCCUUGUCUGGAG |
| siRNA 710 | 710 | UCCAGACAAGGAAGAGUCA | 3608 | UGACUCUUCCUUGUCUGGA |
| siRNA 711 | 711 | CCAGACAAGGAAGAGUCAG | 3609 | CUGACUCUUCCUUGUCUGG |
| siRNA 712 | 712 | CAGACAAGGAAGAGUCAGC | 3610 | GCUGACUCUUCCUUGUCUG |
| siRNA 713 | 713 | AGACAAGGAAGAGUCAGCC | 3611 | GGCUGACUCUUCCUUGUCU |
| siRNA 714 | 714 | GACAAGGAAGAGUCAGCCC | 3612 | GGGCUGACUCUUCCUUGUC |
| siRNA 715 | 715 | ACAAGGAAGAGUCAGCCCC | 3613 | GGGGCUGACUCUUCCUUGU |
| siRNA 716 | 716 | CAAGGAAGAGUCAGCCCCU | 3614 | AGGGGCUGACUCUUCCUUG |
| siRNA 717 | 717 | AAGGAAGAGUCAGCCCCUG | 3615 | CAGGGGCUGACUCUUCCUU |
| siRNA 718 | 718 | AGGAAGAGUCAGCCCCUGC | 3616 | GCAGGGGCUGACUCUUCCU |
| siRNA 719 | 719 | GGAAGAGUCAGCCCCUGCU | 3617 | AGCAGGGGCUGACUCUUCC |
| siRNA 720 | 720 | GAAGAGUCAGCCCCUGCUC | 3618 | GAGCAGGGGCUGACUCUUC |
| siRNA 721 | 721 | AAGAGUCAGCCCCUGCUCC | 3619 | GGAGCAGGGGCUGACUCUU |
| siRNA 722 | 722 | AGAGUCAGCCCCUGCUCCU | 3620 | AGGAGCAGGGGCUGACUCU |
| siRNA 723 | 723 | GAGUCAGCCCCUGCUCCUG | 3621 | CAGGAGCAGGGGCUGACUC |
| siRNA 724 | 724 | AGUCAGCCCCUGCUCCUGG | 3622 | CCAGGAGCAGGGGCUGACU |
| siRNA 725 | 725 | GUCAGCCCCUGCUCCUGGA | 3623 | UCCAGGAGCAGGGGCUGAC |
| siRNA 726 | 726 | UCAGCCCCUGCUCCUGGAC | 3624 | GUCCAGGAGCAGGGGCUGA |
| siRNA 727 | 727 | CAGCCCCUGCUCCUGGACA | 3625 | UGUCCAGGAGCAGGGGCUG |
| siRNA 728 | 728 | AGCCCCUGCUCCUGGACAC | 3626 | GUGUCCAGGAGCAGGGGCU |
| siRNA 729 | 729 | GCCCCUGCUCCUGGACACC | 3627 | GGUGUCCAGGAGCAGGGGC |
| siRNA 730 | 730 | CCCCUGCUCCUGGACACCA | 3628 | UGGUGUCCAGGAGCAGGGG |
| siRNA 731 | 731 | CCCUGCUCCUGGACACCAG | 3629 | CUGGUGUCCAGGAGCAGGG |
| siRNA 732 | 732 | CCUGCUCCUGGACACCAGC | 3630 | GCUGGUGUCCAGGAGCAGG |
| siRNA 733 | 733 | CUGCUCCUGGACACCAGCA | 3631 | UGCUGGUGUCCAGGAGCAG |
| siRNA 734 | 734 | UGCUCCUGGACACCAGCAA | 3632 | UUGCUGGUGUCCAGGAGCA |
| siRNA 735 | 735 | GCUCCUGGACACCAGCAAG | 3633 | CUUGCUGGUGUCCAGGAGC |
| siRNA 736 | 736 | CUCCUGGACACCAGCAAGC | 3634 | GCUUGCUGGUGUCCAGGAG |
| siRNA 737 | 737 | UCCUGGACACCAGCAAGCC | 3635 | GGCUUGCUGGUGUCCAGGA |
| siRNA 738 | 738 | CCUGGACACCAGCAAGCCC | 3636 | GGGCUUGCUGGUGUCCAGG |
| siRNA 739 | 739 | CUGGACACCAGCAAGCCCA | 3637 | UGGGCUUGCUGGUGUCCAG |
| siRNA 740 | 740 | UGGACACCAGCAAGCCCAG | 3638 | CUGGGCUUGCUGGUGUCCA |
| siRNA 741 | 741 | GGACACCAGCAAGCCCAGA | 3639 | UCUGGGCUUGCUGGUGUCC |
| siRNA 742 | 142 | GACACCAGCAAGCCCAGAA | 3640 | UUCUGGGCUUGCUGGUGUC |
| siRNA 743 | 743 | ACACCAGCAAGCCCAGAAG | 3641 | CUUCUGGGCUUGCUGGUGU |
| siRNA 744 | 744 | CACCAGCAAGCCCAGAAGU | 3642 | ACUUCUGGGCUUGCUGGUG |
| siRNA 745 | 745 | ACCAGCAAGCCCAGAAGUC | 3643 | GACUUCUGGGCUUGCUGGU |
| siRNA 746 | 746 | CCAGCAAGCCCAGAAGUCU | 3644 | AGACUUCUGGGCUUGCUGG |
| siRNA 747 | 747 | CAGCAAGCCCAGAAGUCUC | 3645 | GAGACUUCUGGGCUUGCUG |
| siRNA 748 | 748 | AGCAAGCCCAGAAGUCUCC | 3646 | GGAGACUUCUGGGCUUGCU |
| siRNA 749 | 749 | GCAAGCCCAGAAGUCUCCC | 3647 | GGGAGACUUCUGGGCUUGC |
| siRNA 750 | 750 | CAAGCCCAGAAGUCUCCCA | 3648 | UGGGAGACUUCUGGGCUUG |
| siRNA 751 | 751 | AAGCCCAGAAGUCUCCCAA | 3649 | UUGGGAGACUUCUGGGCUU |
| siRNA 752 | 752 | AGCCCAGAAGUCUCCCAAG | 3650 | CUUGGGAGACUUCUGGGCU |
| siRNA 753 | 753 | GCCCAGAAGUCUCCCAAGG | 3651 | CCUUGGGAGACUUCUGGGC |
| siRNA 754 | 754 | CCCAGAAGUCUCCCAAGGC | 3652 | GCCUUGGGAGACUUCUGGG |
| siRNA 755 | 755 | CCAGAAGUCUCCCAAGGCC | 3653 | GGCCUUGGGAGACUUCUGG |
| siRNA 756 | 756 | CAGAAGUCUCCCAAGGCCA | 3654 | UGGCCUUGGGAGACUUCUG |
| siRNA 757 | 757 | AGAAGUCUCCCAAGGCCAA | 3655 | UUGGCCUUGGGAGACUUCU |
| siRNA 758 | 758 | GAAGUCUCCCAAGGCCAAG | 3656 | CUUGGCCUUGGGAGACUUC |
| siRNA 759 | 759 | AAGUCUCCCAAGGCCAAGC | 3657 | GCUUGGCCUUGGGAGACUU |
| siRNA 760 | 760 | AGUCUCCCAAGGCCAAGCC | 3658 | GGCUUGGCCUUGGGAGACU |
| siRNA 761 | 761 | GUCUCCCAAGGCCAAGCCA | 3659 | UGGCUUGGCCUUGGGAGAC |
| siRNA 762 | 762 | UCUCCCAAGGCCAAGCCAA | 3660 | UUGGCUUGGCCUUGGGAGA |
| siRNA 763 | 763 | CUCCCAAGGCCAAGCCAAG | 3661 | CUUGGCUUGGCCUUGGGAG |
| siRNA 764 | 764 | UCCCAAGGCCAAGCCAAGC | 3662 | GCUUGGCUUGGCCUUGGGA |
| siRNA 765 | 765 | CCCAAGGCCAAGCCAAGCC | 3663 | GGCUUGGCUUGGCCUUGGG |
| siRNA 766 | 766 | CCAAGGCCAAGCCAAGCCU | 3664 | AGGCUUGGCUUGGCCUUGG |
| siRNA 767 | 767 | CAAGGCCAAGCCAAGCCUC | 3665 | GAGGCUUGGCUUGGCCUUG |
| siRNA 768 | 768 | AAGGCCAAGCCAAGCCUCU | 3666 | AGAGGCUUGGCUUGGCCUU |
| siRNA 769 | 769 | AGGCCAAGCCAAGCCUCUU | 3667 | AAGAGGCUUGGCUUGGCCU |
| siRNA 770 | 770 | GGCCAAGCCAAGCCUCUUG | 3668 | CAAGAGGCUUGGCUUGGCC |
| siRNA 771 | 771 | GCCAAGCCAAGCCUCUUGA | 3669 | UCAAGAGGCUUGGCUUGGC |
| siRNA 772 | 772 | CCAAGCCAAGCCUCUUGAG | 3670 | CUCAAGAGGCUUGGCUUGG |
| siRNA 773 | 773 | CAAGCCAAGCCUCUUGAGC | 3671 | GCUCAAGAGGCUUGGCUUG |
| siRNA 774 | 774 | AAGCCAAGCCUCUUGAGCA | 3672 | UGCUCAAGAGGCUUGGCUU |
| siRNA 775 | 775 | AGCCAAGCCUCUUGAGCAG | 3673 | CUGCUCAAGAGGCUUGGCU |
| siRNA 776 | 776 | GCCAAGCCUCUUGAGCAGG | 3674 | CCUGCUCAAGAGGCUUGGC |
| siRNA 777 | 777 | CCAAGCCUCUUGAGCAGGG | 3675 | CCCUGCUCAAGAGGCUUGG |
| siRNA 778 | 778 | CAAGCCUCUUGAGCAGGGU | 3676 | ACCCUGCUCAAGAGGCUUG |
| siRNA 779 | 779 | AAGCCUCUUGAGCAGGGUU | 3677 | AACCCUGCUCAAGAGGCUU |
| siRNA 780 | 780 | AGCCUCUUGAGCAGGGUUG | 3678 | CAACCCUGCUCAAGAGGCU |
| siRNA 781 | 781 | GCCUCUUGAGCAGGGUUGG | 3679 | CCAACCCUGCUCAAGAGGC |
| siRNA 782 | 782 | CCUCUUGAGCAGGGUUGGG | 3680 | CCCAACCCUGCUCAAGAGG |
| siRNA 783 | 783 | CUCUUGAGCAGGGUUGGGG | 3681 | CCCCAACCCUGCUCAAGAG |
| siRNA 784 | 784 | UCUUGAGCAGGGUUGGGGC | 3682 | GCCCCAACCCUGCUCAAGA |
| siRNA 785 | 785 | CUUGAGCAGGGUUGGGGCU | 3683 | AGCCCCAACCCUGCUCAAG |
| siRNA 786 | 786 | UUGAGCAGGGUUGGGGCUC | 3684 | GAGCCCCAACCCUGCUCAA |
| siRNA 787 | 787 | UGAGCAGGGUUGGGGCUCU | 3685 | AGAGCCCCAACCCUGCUCA |
| siRNA 788 | 788 | GAGCAGGGUUGGGGCUCUG | 3686 | CAGAGCCCCAACCCUGCUC |
| siRNA 789 | 789 | AGCAGGGUUGGGGCUCUGA | 3687 | UCAGAGCCCCAACCCUGCU |
| siRNA 790 | 790 | GCAGGGUUGGGGCUCUGAC | 3688 | GUCAGAGCCCCAACCCUGC |
| siRNA 791 | 791 | CAGGGUUGGGGCUCUGACC | 3689 | GGUCAGAGCCCCAACCCUG |
| siRNA 792 | 792 | AGGGUUGGGGCUCUGACCA | 3690 | UGGUCAGAGCCCCAACCCU |
| siRNA 793 | 793 | GGGUUGGGGCUCUGACCAA | 3691 | UUGGUCAGAGCCCCAACCC |
| siRNA 794 | 794 | GGUUGGGGCUCUGACCAAC | 3692 | GUUGGUCAGAGCCCCAACC |
| siRNA 795 | 795 | GUUGGGGCUCUGACCAACA | 3693 | UGUUGGUCAGAGCCCCAAC |
| siRNA 796 | 796 | UUGGGGCUCUGACCAACAC | 3694 | GUGUUGGUCAGAGCCCCAA |
| siRNA 797 | 797 | UGGGGCUCUGACCAACACC | 3695 | GGUGUUGGUCAGAGCCCCA |
| siRNA 798 | 798 | GGGGCUCUGACCAACACCC | 3696 | GGGUGUUGGUCAGAGCCCC |
| siRNA 799 | 799 | GGGCUCUGACCAACACCCU | 3697 | AGGGUGUUGGUCAGAGCCC |
| siRNA 800 | 800 | GGCUCUGACCAACACCCUC | 3698 | GAGGGUGUUGGUCAGAGCC |
| siRNA 801 | 801 | GCUCUGACCAACACCCUCU | 3699 | AGAGGGUGUUGGUCAGAGC |
| siRNA 802 | 802 | CUCUGACCAACACCCUCUC | 3700 | GAGAGGGUGUUGGUCAGAG |
| siRNA 803 | 803 | UCUGACCAACACCCUCUCU | 3701 | AGAGAGGGUGUUGGUCAGA |
| siRNA 804 | 804 | CUGACCAACACCCUCUCUC | 3702 | GAGAGAGGGUGUUGGUCAG |
| siRNA 805 | 805 | UGACCAACACCCUCUCUCG | 3703 | CGAGAGAGGGUGUUGGUCA |
| siRNA 806 | 806 | GACCAACACCCUCUCUCGA | 3704 | UCGAGAGAGGGUGUUGGUC |
| siRNA 807 | 807 | ACCAACACCCUCUCUCGAU | 3705 | AUCGAGAGAGGGUGUUGGU |
| siRNA 808 | 808 | CCAACACCCUCUCUCGAUA | 3706 | UAUCGAGAGAGGGUGUUGG |
| siRNA 809 | 809 | CAACACCCUCUCUCGAUAC | 3707 | GUAUCGAGAGAGGGUGUUG |
| siRNA 810 | 810 | AACACCCUCUCUCGAUACA | 3708 | UGUAUCGAGAGAGGGUGUU |
| siRNA 811 | 811 | ACACCCUCUCUCGAUACAC | 3709 | GUGUAUCGAGAGAGGGUGU |
| siRNA 812 | 812 | CACCCUCUCUCGAUACACC | 3710 | GGUGUAUCGAGAGAGGGUG |
| siRNA 813 | 813 | ACCCUCUCUCGAUACACCG | 3711 | CGGUGUAUCGAGAGAGGGU |
| siRNA 814 | 814 | CCCUCUCUCGAUACACCGU | 3712 | ACGGUGUAUCGAGAGAGGG |
| siRNA 815 | 815 | CCUCUCUCGAUACACCGUG | 3713 | CACGGUGUAUCGAGAGAGG |
| siRNA 816 | 816 | CUCUCUCGAUACACCGUGC | 3714 | GCACGGUGUAUCGAGAGAG |
| siRNA 817 | 817 | UCUCUCGAUACACCGUGCA | 3715 | UGCACGGUGUAUCGAGAGA |
| siRNA 818 | 818 | CUCUCGAUACACCGUGCAG | 3716 | CUGCACGGUGUAUCGAGAG |
| siRNA 819 | 819 | UCUCGAUACACCGUGCAGA | 3717 | UCUGCACGGUGUAUCGAGA |
| siRNA 820 | 820 | CUCGAUACACCGUGCAGAC | 3718 | GUCUGCACGGUGUAUCGAG |
| siRNA 821 | 821 | UCGAUACACCGUGCAGACC | 3719 | GGUCUGCACGGUGUAUCGA |
| siRNA 822 | 822 | CGAUACACCGUGCAGACCA | 3720 | UGGUCUGCACGGUGUAUCG |
| siRNA 823 | 823 | GAUACACCGUGCAGACCAU | 3721 | AUGGUCUGCACGGUGUAUC |
| siRNA 824 | 824 | AUACACCGUGCAGACCAUG | 3722 | CAUGGUCUGCACGGUGUAU |
| siRNA 825 | 825 | UACACCGUGCAGACCAUGG | 3723 | CCAUGGUCUGCACGGUGUA |
| siRNA 826 | 826 | ACACCGUGCAGACCAUGGC | 3724 | GCCAUGGUCUGCACGGUGU |
| siRNA 827 | 827 | CACCGUGCAGACCAUGGCC | 3725 | GGCCAUGGUCUGCACGGUG |
| siRNA 828 | 828 | ACCGUGCAGACCAUGGCCC | 3726 | GGGCCAUGGUCUGCACGGU |
| siRNA 829 | 829 | CCGUGCAGACCAUGGCCCG | 3727 | CGGGCCAUGGUCUGCACGG |
| siRNA 830 | 830 | CGUGCAGACCAUGGCCCGG | 3728 | CCGGGCCAUGGUCUGCACG |
| siRNA 831 | 831 | GUGCAGACCAUGGCCCGGG | 3729 | CCCGGGCCAUGGUCUGCAC |
| siRNA 832 | 832 | UGCAGACCAUGGCCCGGGC | 3730 | GCCCGGGCCAUGGUCUGCA |
| siRNA 833 | 833 | GCAGACCAUGGCCCGGGCC | 3731 | GGCCCGGGCCAUGGUCUGC |
| siRNA 834 | 834 | CAGACCAUGGCCCGGGCCC | 3732 | GGGCCCGGGCCAUGGUCUG |
| siRNA 835 | 835 | AGACCAUGGCCCGGGCCCU | 3733 | AGGGCCCGGGCCAUGGUCU |
| siRNA 836 | 836 | GACCAUGGCCCGGGCCCUG | 3734 | CAGGGCCCGGGCCAUGGUC |
| siRNA 837 | 837 | ACCAUGGCCCGGGCCCUGG | 3735 | CCAGGGCCCGGGCCAUGGU |
| siRNA 838 | 838 | CCAUGGCCCGGGCCCUGGA | 3736 | UCCAGGGCCCGGGCCAUGG |
| siRNA 839 | 839 | CAUGGCCCGGGCCCUGGAG | 3737 | CUCCAGGGCCCGGGCCAUG |
| siRNA 840 | 840 | AUGGCCCGGGCCCUGGAGC | 3738 | GCUCCAGGGCCCGGGCCAU |
| siRNA 841 | 841 | UGGCCCGGGCCCUGGAGCA | 3739 | UGCUCCAGGGCCCGGGCCA |
| siRNA 842 | 842 | GGCCCGGGCCCUGGAGCAG | 3740 | CUGCUCCAGGGCCCGGGCC |
| siRNA 843 | 843 | GCCCGGGCCCUGGAGCAGG | 3741 | CCUGCUCCAGGGCCCGGGC |
| siRNA 844 | 844 | CCCGGGCCCUGGAGCAGGG | 3742 | CCCUGCUCCAGGGCCCGGG |
| siRNA 845 | 845 | CCGGGCCCUGGAGCAGGGC | 3743 | GCCCUGCUCCAGGGCCCGG |
| siRNA 846 | 846 | CGGGCCCUGGAGCAGGGCC | 3744 | GGCCCUGCUCCAGGGCCCG |
| siRNA 847 | 847 | GGGCCCUGGAGCAGGGCCA | 3745 | UGGCCCUGCUCCAGGGCCC |
| siRNA 848 | 848 | GGCCCUGGAGCAGGGCCAC | 3746 | GUGGCCCUGCUCCAGGGCC |
| siRNA 849 | 849 | GCCCUGGAGCAGGGCCACA | 3747 | UGUGGCCCUGCUCCAGGGC |
| siRNA 850 | 850 | CCCUGGAGCAGGGCCACAC | 3748 | GUGUGGCCCUGCUCCAGGG |
| siRNA 851 | 851 | CCUGGAGCAGGGCCACACC | 3749 | GGUGUGGCCCUGCUCCAGG |
| siRNA 852 | 852 | CUGGAGCAGGGCCACACCG | 3750 | CGGUGUGGCCCUGCUCCAG |
| siRNA 853 | 853 | UGGAGCAGGGCCACACCGU | 3751 | ACGGUGUGGCCCUGCUCCA |
| siRNA 854 | 854 | GGAGCAGGGCCACACCGUG | 3752 | CACGGUGUGGCCCUGCUCC |
| siRNA 855 | 855 | GAGCAGGGCCACACCGUGG | 3753 | CCACGGUGUGGCCCUGCUC |
| siRNA 856 | 856 | AGCAGGGCCACACCGUGGC | 3754 | GCCACGGUGUGGCCCUGCU |
| siRNA 857 | 857 | GCAGGGCCACACCGUGGCC | 3755 | GGCCACGGUGUGGCCCUGC |
| siRNA 858 | 858 | CAGGGCCACACCGUGGCCA | 3756 | UGGCCACGGUGUGGCCCUG |
| siRNA 859 | 859 | AGGGCCACACCGUGGCCAU | 3757 | AUGGCCACGGUGUGGCCCU |
| siRNA 860 | 860 | GGGCCACACCGUGGCCAUG | 3758 | CAUGGCCACGGUGUGGCCC |
| siRNA 861 | 861 | GGCCACACCGUGGCCAUGU | 3759 | ACAUGGCCACGGUGUGGCC |
| siRNA 862 | 862 | GCCACACCGUGGCCAUGUG | 3760 | CACAUGGCCACGGUGUGGC |
| siRNA 863 | 863 | CCACACCGUGGCCAUGUGG | 3761 | CCACAUGGCCACGGUGUGG |
| siRNA 864 | 864 | CACACCGUGGCCAUGUGGA | 3762 | UCCACAUGGCCACGGUGUG |
| siRNA 865 | 865 | ACACCGUGGCCAUGUGGAU | 3763 | AUCCACAUGGCCACGGUGU |
| siRNA 866 | 866 | CACCGUGGCCAUGUGGAUC | 3764 | GAUCCACAUGGCCACGGUG |
| siRNA 867 | 867 | ACCGUGGCCAUGUGGAUCC | 3765 | GGAUCCACAUGGCCACGGU |
| siRNA 868 | 868 | CCGUGGCCAUGUGGAUCCC | 3766 | GGGAUCCACAUGGCCACGG |
| siRNA 869 | 869 | CGUGGCCAUGUGGAUCCCA | 3767 | UGGGAUCCACAUGGCCACG |
| siRNA 870 | 870 | GUGGCCAUGUGGAUCCCAG | 3768 | CUGGGAUCCACAUGGCCAC |
| siRNA 871 | 871 | UGGCCAUGUGGAUCCCAGG | 3769 | CCUGGGAUCCACAUGGCCA |
| siRNA 872 | 872 | GGCCAUGUGGAUCCCAGGC | 3770 | GCCUGGGAUCCACAUGGCC |
| siRNA 873 | 873 | GCCAUGUGGAUCCCAGGCG | 3771 | CGCCUGGGAUCCACAUGGC |
| siRNA 874 | 874 | CCAUGUGGAUCCCAGGCGU | 3772 | ACGCCUGGGAUCCACAUGG |
| siRNA 875 | 875 | CAUGUGGAUCCCAGGCGUG | 3773 | CACGCCUGGGAUCCACAUG |
| siRNA 876 | 876 | AUGUGGAUCCCAGGCGUGG | 3774 | CCACGCCUGGGAUCCACAU |
| siRNA 877 | 877 | UGUGGAUCCCAGGCGUGGU | 3775 | ACCACGCCUGGGAUCCACA |
| siRNA 878 | 878 | GUGGAUCCCAGGCGUGGUG | 3776 | CACCACGCCUGGGAUCCAC |
| siRNA 879 | 879 | UGGAUCCCAGGCGUGGUGC | 3777 | GCACCACGCCUGGGAUCCA |
| siRNA 880 | 880 | GGAUCCCAGGCGUGGUGCC | 3778 | GGCACCACGCCUGGGAUCC |
| siRNA 881 | 881 | GAUCCCAGGCGUGGUGCCC | 3779 | GGGCACCACGCCUGGGAUC |
| siRNA 882 | 882 | AUCCCAGGCGUGGUGCCCC | 3780 | GGGGCACCACGCCUGGGAU |
| siRNA 883 | 883 | UCCCAGGCGUGGUGCCCCU | 3781 | AGGGGCACCACGCCUGGGA |
| siRNA 884 | 884 | CCCAGGCGUGGUGCCCCUG | 3782 | CAGGGGCACCACGCCUGGG |
| siRNA 885 | 885 | CCAGGCGUGGUGCCCCUGA | 3783 | UCAGGGGCACCACGCCUGG |
| siRNA 886 | 886 | CAGGCGUGGUGCCCCUGAG | 3784 | CUCAGGGGCACCACGCCUG |
| siRNA 887 | 887 | AGGCGUGGUGCCCCUGAGC | 3785 | GCUCAGGGGCACCACGCCU |
| siRNA 888 | 888 | GGCGUGGUGCCCCUGAGCA | 3786 | UGCUCAGGGGCACCACGCC |
| siRNA 889 | 889 | GCGUGGUGCCCCUGAGCAG | 3787 | CUGCUCAGGGGCACCACGC |
| siRNA 890 | 890 | CGUGGUGCCCCUGAGCAGC | 3788 | GCUGCUCAGGGGCACCACG |
| siRNA 891 | 891 | GUGGUGCCCCUGAGCAGCC | 3789 | GGCUGCUCAGGGGCACCAC |
| siRNA 892 | 892 | UGGUGCCCCUGAGCAGCCU | 3790 | AGGCUGCUCAGGGGCACCA |
| siRNA 893 | 893 | GGUGCCCCUGAGCAGCCUG | 3791 | CAGGCUGCUCAGGGGCACC |
| siRNA 894 | 894 | GUGCCCCUGAGCAGCCUGG | 3792 | CCAGGCUGCUCAGGGGCAC |
| siRNA 895 | 895 | UGCCCCUGAGCAGCCUGGC | 3793 | GCCAGGCUGCUCAGGGGCA |
| siRNA 896 | 896 | GCCCCUGAGCAGCCUGGCC | 3794 | GGCCAGGCUGCUCAGGGGC |
| siRNA 897 | 897 | CCCCUGAGCAGCCUGGCCC | 3795 | GGGCCAGGCUGCUCAGGGG |
| siRNA 898 | 898 | CCCUGAGCAGCCUGGCCCA | 3796 | UGGGCCAGGCUGCUCAGGG |
| siRNA 899 | 899 | CCUGAGCAGCCUGGCCCAG | 3797 | CUGGGCCAGGCUGCUCAGG |
| siRNA 900 | 900 | CUGAGCAGCCUGGCCCAGU | 3798 | ACUGGGCCAGGCUGCUCAG |
| siRNA 901 | 901 | UGAGCAGCCUGGCCCAGUG | 3799 | CACUGGGCCAGGCUGCUCA |
| siRNA 902 | 902 | GAGCAGCCUGGCCCAGUGG | 3800 | CCACUGGGCCAGGCUGCUC |
| siRNA 903 | 903 | AGCAGCCUGGCCCAGUGGG | 3801 | CCCACUGGGCCAGGCUGCU |
| siRNA 904 | 904 | GCAGCCUGGCCCAGUGGGG | 3802 | CCCCACUGGGCCAGGCUGC |
| siRNA 905 | 905 | CAGCCUGGCCCAGUGGGGU | 3803 | ACCCCACUGGGCCAGGCUG |
| siRNA 906 | 906 | AGCCUGGCCCAGUGGGGUG | 3804 | CACCCCACUGGGCCAGGCU |
| siRNA 907 | 907 | GCCUGGCCCAGUGGGGUGC | 3805 | GCACCCCACUGGGCCAGGC |
| siRNA 908 | 908 | CCUGGCCCAGUGGGGUGCC | 3806 | GGCACCCCACUGGGCCAGG |
| siRNA 909 | 909 | CUGGCCCAGUGGGGUGCCU | 3807 | AGGCACCCCACUGGGCCAG |
| siRNA 910 | 910 | UGGCCCAGUGGGGUGCCUC | 3808 | GAGGCACCCCACUGGGCCA |
| siRNA 911 | 911 | GGCCCAGUGGGGUGCCUCA | 3809 | UGAGGCACCCCACUGGGCC |
| siRNA 912 | 912 | GCCCAGUGGGGUGCCUCAG | 3810 | CUGAGGCACCCCACUGGGC |
| siRNA 913 | 913 | CCCAGUGGGGUGCCUCAGU | 3811 | ACUGAGGCACCCCACUGGG |
| siRNA 914 | 914 | CCAGUGGGGUGCCUCAGUG | 3812 | CACUGAGGCACCCCACUGG |
| siRNA 915 | 915 | CAGUGGGGUGCCUCAGUGG | 3813 | CCACUGAGGCACCCCACUG |
| siRNA 916 | 916 | AGUGGGGUGCCUCAGUGGC | 3814 | GCCACUGAGGCACCCCACU |
| siRNA 917 | 917 | GUGGGGUGCCUCAGUGGCC | 3815 | GGCCACUGAGGCACCCCAC |
| siRNA 918 | 918 | UGGGGUGCCUCAGUGGCCA | 3816 | UGGCCACUGAGGCACCCCA |
| SIRNA 919 | 919 | GGGGUGCCUCAGUGGCCAU | 3817 | AUGGCCACUGAGGCACCCC |
| siRNA 920 | 920 | GGGUGCCUCAGUGGCCAUG | 3818 | CAUGGCCACUGAGGCACCC |
| siRNA 921 | 921 | GGUGCCUCAGUGGCCAUGC | 3819 | GCAUGGCCACUGAGGCACC |
| siRNA 922 | 922 | GUGCCUCAGUGGCCAUGCA | 3820 | UGCAUGGCCACUGAGGCAC |
| siRNA 923 | 923 | UGCCUCAGUGGCCAUGCAG | 3821 | CUGCAUGGCCACUGAGGCA |
| siRNA 924 | 924 | GCCUCAGUGGCCAUGCAGG | 3822 | CCUGCAUGGCCACUGAGGC |
| siRNA 925 | 925 | CCUCAGUGGCCAUGCAGGC | 3823 | GCCUGCAUGGCCACUGAGG |
| siRNA 926 | 926 | CUCAGUGGCCAUGCAGGCG | 3824 | CGCCUGCAUGGCCACUGAG |
| siRNA 927 | 927 | UCAGUGGCCAUGCAGGCGG | 3825 | CCGCCUGCAUGGCCACUGA |
| siRNA 928 | 928 | CAGUGGCCAUGCAGGCGGU | 3826 | ACCGCCUGCAUGGCCACUG |
| siRNA 929 | 929 | AGUGGCCAUGCAGGCGGUG | 3827 | CACCGCCUGCAUGGCCACU |
| siRNA 930 | 930 | GUGGCCAUGCAGGCGGUGU | 3828 | ACACCGCCUGCAUGGCCAC |
| siRNA 931 | 931 | UGGCCAUGCAGGCGGUGUC | 3829 | GACACCGCCUGCAUGGCCA |
| siRNA 932 | 932 | GGCCAUGCAGGCGGUGUCC | 3830 | GGACACCGCCUGCAUGGCC |
| siRNA 933 | 933 | GCCAUGCAGGCGGUGUCCC | 3831 | GGGACACCGCCUGCAUGGC |
| siRNA 934 | 934 | CCAUGCAGGCGGUGUCCCG | 3832 | CGGGACACCGCCUGCAUGG |
| siRNA 935 | 935 | CAUGCAGGCGGUGUCCCGG | 3833 | CCGGGACACCGCCUGCAUG |
| siRNA 936 | 936 | AUGCAGGCGGUGUCCCGGC | 3834 | GCCGGGACACCGCCUGCAU |
| siRNA 937 | 937 | UGCAGGCGGUGUCCCGGCG | 3835 | CGCCGGGACACCGCCUGCA |
| siRNA 938 | 938 | GCAGGCGGUGUCCCGGCGG | 3836 | CCGCCGGGACACCGCCUGC |
| siRNA 939 | 939 | CAGGCGGUGUCCCGGCGGA | 3837 | UCCGCCGGGACACCGCCUG |
| siRNA 940 | 940 | AGGCGGUGUCCCGGCGGAG | 3838 | CUCCGCCGGGACACCGCCU |
| siRNA 941 | 941 | GGCGGUGUCCCGGCGGAGG | 3839 | CCUCCGCCGGGACACCGCC |
| siRNA 942 | 942 | GCGGUGUCCCGGCGGAGGA | 3840 | UCCUCCGCCGGGACACCGC |
| siRNA 943 | 943 | CGGUGUCCCGGCGGAGGAG | 3841 | CUCCUCCGCCGGGACACCG |
| siRNA 944 | 944 | GGUGUCCCGGCGGAGGAGC | 3842 | GCUCCUCCGCCGGGACACC |
| siRNA 945 | 945 | GUGUCCCGGCGGAGGAGCG | 3843 | CGCUCCUCCGCCGGGACAC |
| siRNA 946 | 946 | UGUCCCGGCGGAGGAGCGA | 3844 | UCGCUCCUCCGCCGGGACA |
| siRNA 947 | 947 | GUCCCGGCGGAGGAGCGAA | 3845 | UUCGCUCCUCCGCCGGGAC |
| siRNA 948 | 948 | UCCCGGCGGAGGAGCGAAG | 3846 | CUUCGCUCCUCCGCCGGGA |
| siRNA 949 | 949 | CCCGGCGGAGGAGCGAAGU | 3847 | ACUUCGCUCCUCCGCCGGG |
| siRNA 950 | 950 | CCGGCGGAGGAGCGAAGUG | 3848 | CACUUCGCUCCUCCGCCGG |
| siRNA 951 | 951 | CGGCGGAGGAGCGAAGUGC | 3849 | GCACUUCGCUCCUCCGCCG |
| siRNA 952 | 952 | GGCGGAGGAGCGAAGUGCG | 3850 | CGCACUUCGCUCCUCCGCC |
| siRNA 953 | 953 | GCGGAGGAGCGAAGUGCGG | 3851 | CCGCACUUCGCUCCUCCGC |
| siRNA 954 | 954 | CGGAGGAGCGAAGUGCGGG | 3852 | CCCGCACUUCGCUCCUCCG |
| siRNA 955 | 955 | GGAGGAGCGAAGUGCGGGU | 3853 | ACCCGCACUUCGCUCCUCC |
| siRNA 956 | 956 | GAGGAGCGAAGUGCGGGUA | 3854 | UACCCGCACUUCGCUCCUC |
| siRNA 957 | 957 | AGGAGCGAAGUGCGGGUAC | 3855 | GUACCCGCACUUCGCUCCU |
| siRNA 958 | 958 | GGAGCGAAGUGCGGGUACC | 3856 | GGUACCCGCACUUCGCUCC |
| siRNA 959 | 959 | GAGCGAAGUGCGGGUACCC | 3857 | GGGUACCCGCACUUCGCUC |
| siRNA 960 | 960 | AGCGAAGUGCGGGUACCCU | 3858 | AGGGUACCCGCACUUCGCU |
| siRNA 961 | 961 | GCGAAGUGCGGGUACCCUG | 3859 | CAGGGUACCCGCACUUCGC |
| siRNA 962 | 962 | CGAAGUGCGGGUACCCUGG | 3860 | CCAGGGUACCCGCACUUCG |
| siRNA 963 | 963 | GAAGUGCGGGUACCCUGGC | 3861 | GCCAGGGUACCCGCACUUC |
| siRNA 964 | 964 | AAGUGCGGGUACCCUGGCU | 3862 | AGCCAGGGUACCCGCACUU |
| siRNA 965 | 965 | AGUGCGGGUACCCUGGCUG | 3863 | CAGCCAGGGUACCCGCACU |
| siRNA 966 | 966 | GUGCGGGUACCCUGGCUGC | 3864 | GCAGCCAGGGUACCCGCAC |
| siRNA 967 | 967 | UGCGGGUACCCUGGCUGCA | 3865 | UGCAGCCAGGGUACCCGCA |
| siRNA 968 | 968 | GCGGGUACCCUGGCUGCAC | 3866 | GUGCAGCCAGGGUACCCGC |
| siRNA 969 | 969 | CGGGUACCCUGGCUGCACA | 3867 | UGUGCAGCCAGGGUACCCG |
| siRNA 970 | 970 | GGGUACCCUGGCUGCACAG | 3868 | CUGUGCAGCCAGGGUACCC |
| siRNA 971 | 971 | GGUACCCUGGCUGCACAGC | 3869 | GCUGUGCAGCCAGGGUACC |
| siRNA 972 | 972 | GUACCCUGGCUGCACAGCC | 3870 | GGCUGUGCAGCCAGGGUAC |
| siRNA 973 | 973 | UACCCUGGCUGCACAGCCU | 3871 | AGGCUGUGCAGCCAGGGUA |
| siRNA 974 | 974 | ACCCUGGCUGCACAGCCUC | 3872 | GAGGCUGUGCAGCCAGGGU |
| siRNA 975 | 975 | CCCUGGCUGCACAGCCUCG | 3873 | CGAGGCUGUGCAGCCAGGG |
| siRNA 976 | 976 | CCUGGCUGCACAGCCUCGC | 3874 | GCGAGGCUGUGCAGCCAGG |
| siRNA 977 | 977 | CUGGCUGCACAGCCUCGCA | 3875 | UGCGAGGCUGUGCAGCCAG |
| siRNA 978 | 978 | UGGCUGCACAGCCUCGCAG | 3876 | CUGCGAGGCUGUGCAGCCA |
| siRNA 979 | 979 | GGCUGCACAGCCUCGCAGC | 3877 | GCUGCGAGGCUGUGCAGCC |
| siRNA 980 | 980 | GCUGCACAGCCUCGCAGCC | 3878 | GGCUGCGAGGCUGUGCAGC |
| siRNA 981 | 981 | CUGCACAGCCUCGCAGCCG | 3879 | CGGCUGCGAGGCUGUGCAG |
| siRNA 982 | 982 | UGCACAGCCUCGCAGCCGC | 3880 | GCGGCUGCGAGGCUGUGCA |
| siRNA 983 | 983 | GCACAGCCUCGCAGCCGCC | 3881 | GGCGGCUGCGAGGCUGUGC |
| siRNA 984 | 984 | CACAGCCUCGCAGCCGCCC | 3882 | GGGCGGCUGCGAGGCUGUG |
| siRNA 985 | 985 | ACAGCCUCGCAGCCGCCCA | 3883 | UGGGCGGCUGCGAGGCUGU |
| siRNA 986 | 986 | CAGCCUCGCAGCCGCCCAG | 3884 | CUGGGCGGCUGCGAGGCUG |
| siRNA 987 | 987 | AGCCUCGCAGCCGCCCAGG | 3885 | CCUGGGCGGCUGCGAGGCU |
| siRNA 988 | 988 | GCCUCGCAGCCGCCCAGGA | 3886 | UCCUGGGCGGCUGCGAGGC |
| siRNA 989 | 989 | CCUCGCAGCCGCCCAGGAG | 3887 | CUCCUGGGCGGCUGCGAGG |
| siRNA 990 | 990 | CUCGCAGCCGCCCAGGAGG | 3888 | CCUCCUGGGCGGCUGCGAG |
| siRNA 991 | 991 | UCGCAGCCGCCCAGGAGGA | 3889 | UCCUCCUGGGCGGCUGCGA |
| siRNA 992 | 992 | CGCAGCCGCCCAGGAGGAG | 3890 | CUCCUCCUGGGCGGCUGCG |
| siRNA 993 | 993 | GCAGCCGCCCAGGAGGAGG | 3891 | CCUCCUCCUGGGCGGCUGC |
| siRNA 994 | 994 | CAGCCGCCCAGGAGGAGGA | 3892 | UCCUCCUCCUGGGCGGCUG |
| siRNA 995 | 995 | AGCCGCCCAGGAGGAGGAU | 3893 | AUCCUCCUCCUGGGCGGCU |
| siRNA 996 | 996 | GCCGCCCAGGAGGAGGAUC | 3894 | GAUCCUCCUCCUGGGCGGC |
| siRNA 997 | 997 | CCGCCCAGGAGGAGGAUCA | 3895 | UGAUCCUCCUCCUGGGCGG |
| siRNA 998 | 998 | CGCCCAGGAGGAGGAUCAU | 3896 | AUGAUCCUCCUCCUGGGCG |
| siRNA 999 | 999 | GCCCAGGAGGAGGAUCAUG | 3897 | CAUGAUCCUCCUCCUGGGC |
| siRNA 1000 | 1000 | CCCAGGAGGAGGAUCAUGA | 3898 | UCAUGAUCCUCCUCCUGGG |
| siRNA 1001 | 1001 | CCAGGAGGAGGAUCAUGAG | 3899 | CUCAUGAUCCUCCUCCUGG |
| siRNA 1002 | 1002 | CAGGAGGAGGAUCAUGAGG | 3900 | CCUCAUGAUCCUCCUCCUG |
| siRNA 1003 | 1003 | AGGAGGAGGAUCAUGAGGA | 3901 | UCCUCAUGAUCCUCCUCCU |
| siRNA 1004 | 1004 | GGAGGAGGAUCAUGAGGAC | 3902 | GUCCUCAUGAUCCUCCUCC |
| siRNA 1005 | 1005 | GAGGAGGAUCAUGAGGACC | 3903 | GGUCCUCAUGAUCCUCCUC |
| siRNA 1006 | 1006 | AGGAGGAUCAUGAGGACCA | 3904 | UGGUCCUCAUGAUCCUCCU |
| siRNA 1007 | 1007 | GGAGGAUCAUGAGGACCAG | 3905 | CUGGUCCUCAUGAUCCUCC |
| siRNA 1008 | 1008 | GAGGAUCAUGAGGACCAGA | 3906 | UCUGGUCCUCAUGAUCCUC |
| siRNA 1009 | 1009 | AGGAUCAUGAGGACCAGAC | 3907 | GUCUGGUCCUCAUGAUCCU |
| siRNA 1010 | 1010 | GGAUCAUGAGGACCAGACA | 3908 | UGUCUGGUCCUCAUGAUCC |
| siRNA 1011 | 1011 | GAUCAUGAGGACCAGACAG | 3909 | CUGUCUGGUCCUCAUGAUC |
| siRNA 1012 | 1012 | AUCAUGAGGACCAGACAGA | 3910 | UCUGUCUGGUCCUCAUGAU |
| siRNA 1013 | 1013 | UCAUGAGGACCAGACAGAC | 3911 | GUCUGUCUGGUCCUCAUGA |
| siRNA 1014 | 1014 | CAUGAGGACCAGACAGACA | 3912 | UGUCUGUCUGGUCCUCAUG |
| siRNA 1015 | 1015 | AUGAGGACCAGACAGACAC | 3913 | GUGUCUGUCUGGUCCUCAU |
| siRNA 1016 | 1016 | UGAGGACCAGACAGACACG | 3914 | CGUGUCUGUCUGGUCCUCA |
| siRNA 1017 | 1017 | GAGGACCAGACAGACACGG | 3915 | CCGUGUCUGUCUGGUCCUC |
| siRNA 1018 | 1018 | AGGACCAGACAGACACGGA | 3916 | UCCGUGUCUGUCUGGUCCU |
| siRNA 1019 | 1019 | GGACCAGACAGACACGGAG | 3917 | CUCCGUGUCUGUCUGGUCC |
| siRNA 1020 | 1020 | GACCAGACAGACACGGAGG | 3918 | CCUCCGUGUCUGUCUGGUC |
| siRNA 1021 | 1021 | ACCAGACAGACACGGAGGG | 3919 | CCCUCCGUGUCUGUCUGGU |
| siRNA 1022 | 1022 | CCAGACAGACACGGAGGGA | 3920 | UCCCUCCGUGUCUGUCUGG |
| siRNA 1023 | 1023 | CAGACAGACACGGAGGGAG | 3921 | CUCCCUCCGUGUCUGUCUG |
| siRNA 1024 | 1024 | AGACAGACACGGAGGGAGA | 3922 | UCUCCCUCCGUGUCUGUCU |
| siRNA 1025 | 1025 | GACAGACACGGAGGGAGAG | 3923 | CUCUCCCUCCGUGUCUGUC |
| siRNA 1026 | 1026 | ACAGACACGGAGGGAGAGG | 3924 | CCUCUCCCUCCGUGUCUGU |
| siRNA 1027 | 1027 | CAGACACGGAGGGAGAGGA | 3925 | UCCUCUCCCUCCGUGUCUG |
| siRNA 1028 | 1028 | AGACACGGAGGGAGAGGAC | 3926 | GUCCUCUCCCUCCGUGUCU |
| siRNA 1029 | 1029 | GACACGGAGGGAGAGGACA | 3927 | UGUCCUCUCCCUCCGUGUC |
| siRNA 1030 | 1030 | ACACGGAGGGAGAGGACAC | 3928 | GUGUCCUCUCCCUCCGUGU |
| siRNA 1031 | 1031 | CACGGAGGGAGAGGACACG | 3929 | CGUGUCCUCUCCCUCCGUG |
| siRNA 1032 | 1032 | ACGGAGGGAGAGGACACGG | 3930 | CCGUGUCCUCUCCCUCCGU |
| siRNA 1033 | 1033 | CGGAGGGAGAGGACACGGA | 3931 | UCCGUGUCCUCUCCCUCCG |
| siRNA 1034 | 1034 | GGAGGGAGAGGACACGGAG | 3932 | CUCCGUGUCCUCUCCCUCC |
| siRNA 1035 | 1035 | GAGGGAGAGGACACGGAGG | 3933 | CCUCCGUGUCCUCUCCCUC |
| siRNA 1036 | 1036 | AGGGAGAGGACACGGAGGA | 3934 | UCCUCCGUGUCCUCUCCCU |
| siRNA 1037 | 1037 | GGGAGAGGACACGGAGGAG | 3935 | CUCCUCCGUGUCCUCUCCC |
| siRNA 1038 | 1038 | GGAGAGGACACGGAGGAGG | 3936 | CCUCCUCCGUGUCCUCUCC |
| siRNA 1039 | 1039 | GAGAGGACACGGAGGAGGA | 3937 | UCCUCCUCCGUGUCCUCUC |
| siRNA 1040 | 1040 | AGAGGACACGGAGGAGGAG | 3938 | CUCCUCCUCCGUGUCCUCU |
| siRNA 1041 | 1041 | GAGGACACGGAGGAGGAGG | 3939 | CCUCCUCCUCCGUGUCCUC |
| siRNA 1042 | 1042 | AGGACACGGAGGAGGAGGA | 3940 | UCCUCCUCCUCCGUGUCCU |
| siRNA 1043 | 1043 | GGACACGGAGGAGGAGGAA | 3941 | UUCCUCCUCCUCCGUGUCC |
| siRNA 1044 | 1044 | GACACGGAGGAGGAGGAAG | 3942 | CUUCCUCCUCCUCCGUGUC |
| siRNA 1045 | 1045 | ACACGGAGGAGGAGGAAGA | 3943 | UCUUCCUCCUCCUCCGUGU |
| siRNA 1046 | 1046 | CACGGAGGAGGAGGAAGAA | 3944 | UUCUUCCUCCUCCUCCGUG |
| siRNA 1047 | 1047 | ACGGAGGAGGAGGAAGAAU | 3945 | AUUCUUCCUCCUCCUCCGU |
| siRNA 1048 | 1048 | CGGAGGAGGAGGAAGAAUU | 3946 | AAUUCUUCCUCCUCCUCCG |
| siRNA 1049 | 1049 | GGAGGAGGAGGAAGAAUUG | 3947 | CAAUUCUUCCUCCUCCUCC |
| siRNA 1050 | 1050 | GAGGAGGAGGAAGAAUUGG | 3948 | CCAAUUCUUCCUCCUCCUC |
| siRNA 1051 | 1051 | AGGAGGAGGAAGAAUUGGA | 3949 | UCCAAUUCUUCCUCCUCCU |
| siRNA 1052 | 1052 | GGAGGAGGAAGAAUUGGAG | 3950 | CUCCAAUUCUUCCUCCUCC |
| siRNA 1053 | 1053 | GAGGAGGAAGAAUUGGAGA | 3951 | UCUCCAAUUCUUCCUCCUC |
| siRNA 1054 | 1054 | AGGAGGAAGAAUUGGAGAC | 3952 | GUCUCCAAUUCUUCCUCCU |
| siRNA 1055 | 1055 | GGAGGAAGAAUUGGAGACU | 3953 | AGUCUCCAAUUCUUCCUCC |
| siRNA 1056 | 1056 | GAGGAAGAAUUGGAGACUG | 3954 | CAGUCUCCAAUUCUUCCUC |
| siRNA 1057 | 1057 | AGGAAGAAUUGGAGACUGA | 3955 | UCAGUCUCCAAUUCUUCCU |
| siRNA 1058 | 1058 | GGAAGAAUUGGAGACUGAG | 3956 | CUCAGUCUCCAAUUCUUCC |
| siRNA 1059 | 1059 | GAAGAAUUGGAGACUGAGG | 3957 | CCUCAGUCUCCAAUUCUUC |
| siRNA 1060 | 1060 | AAGAAUUGGAGACUGAGGA | 3958 | UCCUCAGUCUCCAAUUCUU |
| siRNA 1061 | 1061 | AGAAUUGGAGACUGAGGAG | 3959 | CUCCUCAGUCUCCAAUUCU |
| siRNA 1062 | 1062 | GAAUUGGAGACUGAGGAGA | 3960 | UCUCCUCAGUCUCCAAUUC |
| siRNA 1063 | 1063 | AAUUGGAGACUGAGGAGAA | 3961 | UUCUCCUCAGUCUCCAAUU |
| siRNA 1064 | 1064 | AUUGGAGACUGAGGAGAAC | 3962 | GUUCUCCUCAGUCUCCAAU |
| siRNA 1065 | 1065 | UUGGAGACUGAGGAGAACA | 3963 | UGUUCUCCUCAGUCUCCAA |
| siRNA 1066 | 1066 | UGGAGACUGAGGAGAACAA | 3964 | UUGUUCUCCUCAGUCUCCA |
| siRNA 1067 | 1067 | GGAGACUGAGGAGAACAAG | 3965 | CUUGUUCUCCUCAGUCUCC |
| siRNA 1068 | 1068 | GAGACUGAGGAGAACAAGU | 3966 | ACUUGUUCUCCUCAGUCUC |
| siRNA 1069 | 1069 | AGACUGAGGAGAACAAGUU | 3967 | AACUUGUUCUCCUCAGUCU |
| siRNA 1070 | 1070 | GACUGAGGAGAACAAGUUC | 3968 | GAACUUGUUCUCCUCAGUC |
| siRNA 1071 | 1071 | ACUGAGGAGAACAAGUUCA | 3969 | UGAACUUGUUCUCCUCAGU |
| siRNA 1072 | 1072 | CUGAGGAGAACAAGUUCAG | 3970 | CUGAACUUGUUCUCCUCAG |
| siRNA 1073 | 1073 | UGAGGAGAACAAGUUCAGU | 3971 | ACUGAACUUGUUCUCCUCA |
| siRNA 1074 | 1074 | GAGGAGAACAAGUUCAGUG | 3972 | CACUGAACUUGUUCUCCUC |
| siRNA 1075 | 1075 | AGGAGAACAAGUUCAGUGA | 3973 | UCACUGAACUUGUUCUCCU |
| siRNA 1076 | 1076 | GGAGAACAAGUUCAGUGAG | 3974 | CUCACUGAACUUGUUCUCC |
| siRNA 1077 | 1077 | GAGAACAAGUUCAGUGAGG | 3975 | CCUCACUGAACUUGUUCUC |
| siRNA 1078 | 1078 | AGAACAAGUUCAGUGAGGU | 3976 | ACCUCACUGAACUUGUUCU |
| siRNA 1079 | 1079 | GAACAAGUUCAGUGAGGUA | 3977 | UACCUCACUGAACUUGUUC |
| siRNA 1080 | 1080 | AACAAGUUCAGUGAGGUAG | 3978 | CUACCUCACUGAACUUGUU |
| siRNA 1081 | 1081 | ACAAGUUCAGUGAGGUAGC | 3979 | GCUACCUCACUGAACUUGU |
| siRNA 1082 | 1082 | CAAGUUCAGUGAGGUAGCA | 3980 | UGCUACCUCACUGAACUUG |
| siRNA 1083 | 1083 | AAGUUCAGUGAGGUAGCAG | 3981 | CUGCUACCUCACUGAACUU |
| siRNA 1084 | 1084 | AGUUCAGUGAGGUAGCAGC | 3982 | GCUGCUACCUCACUGAACU |
| siRNA 1085 | 1085 | GUUCAGUGAGGUAGCAGCC | 3983 | GGCUGCUACCUCACUGAAC |
| siRNA 1086 | 1086 | UUCAGUGAGGUAGCAGCCC | 3984 | GGGCUGCUACCUCACUGAA |
| siRNA 1087 | 1087 | UCAGUGAGGUAGCAGCCCU | 3985 | AGGGCUGCUACCUCACUGA |
| siRNA 1088 | 1088 | CAGUGAGGUAGCAGCCCUG | 3986 | CAGGGCUGCUACCUCACUG |
| siRNA 1089 | 1089 | AGUGAGGUAGCAGCCCUGC | 3987 | GCAGGGCUGCUACCUCACU |
| siRNA 1090 | 1090 | GUGAGGUAGCAGCCCUGCC | 3988 | GGCAGGGCUGCUACCUCAC |
| siRNA 1091 | 1091 | UGAGGUAGCAGCCCUGCCA | 3989 | UGGCAGGGCUGCUACCUCA |
| siRNA 1092 | 1092 | GAGGUAGCAGCCCUGCCAG | 3990 | CUGGCAGGGCUGCUACCUC |
| siRNA 1093 | 1093 | AGGUAGCAGCCCUGCCAGG | 3991 | CCUGGCAGGGCUGCUACCU |
| siRNA 1094 | 1094 | GGUAGCAGCCCUGCCAGGC | 3992 | GCCUGGCAGGGCUGCUACC |
| siRNA 1095 | 1095 | GUAGCAGCCCUGCCAGGCC | 3993 | GGCCUGGCAGGGCUGCUAC |
| siRNA 1096 | 1096 | UAGCAGCCCUGCCAGGCCC | 3994 | GGGCCUGGCAGGGCUGCUA |
| siRNA 1097 | 1097 | AGCAGCCCUGCCAGGCCCU | 3995 | AGGGCCUGGCAGGGCUGCU |
| siRNA 1098 | 1098 | GCAGCCCUGCCAGGCCCUC | 3996 | GAGGGCCUGGCAGGGCUGC |
| siRNA 1099 | 1099 | CAGCCCUGCCAGGCCCUCG | 3997 | CGAGGGCCUGGCAGGGCUG |
| siRNA 1100 | 1100 | AGCCCUGCCAGGCCCUCGA | 3998 | UCGAGGGCCUGGCAGGGCU |
| siRNA 1101 | 1101 | GCCCUGCCAGGCCCUCGAG | 3999 | CUCGAGGGCCUGGCAGGGC |
| siRNA 1102 | 1102 | CCCUGCCAGGCCCUCGAGG | 4000 | CCUCGAGGGCCUGGCAGGG |
| siRNA 1103 | 1103 | CCUGCCAGGCCCUCGAGGC | 4001 | GCCUCGAGGGCCUGGCAGG |
| siRNA 1104 | 1104 | CUGCCAGGCCCUCGAGGCC | 4002 | GGCCUCGAGGGCCUGGCAG |
| SIRNA 1105 | 1105 | UGCCAGGCCCUCGAGGCCU | 4003 | AGGCCUCGAGGGCCUGGCA |
| siRNA 1106 | 1106 | GCCAGGCCCUCGAGGCCUC | 4004 | GAGGCCUCGAGGGCCUGGC |
| siRNA 1107 | 1107 | CCAGGCCCUCGAGGCCUCC | 4005 | GGAGGCCUCGAGGGCCUGG |
| siRNA 1108 | 1108 | CAGGCCCUCGAGGCCUCCU | 4006 | AGGAGGCCUCGAGGGCCUG |
| siRNA 1109 | 1109 | AGGCCCUCGAGGCCUCCUG | 4007 | CAGGAGGCCUCGAGGGCCU |
| siRNA 1110 | 1110 | GGCCCUCGAGGCCUCCUGG | 4008 | CCAGGAGGCCUCGAGGGCC |
| SiRNA 1111 | 1111 | GCCCUCGAGGCCUCCUGGG | 4009 | CCCAGGAGGCCUCGAGGGC |
| siRNA 1112 | 1112 | CCCUCGAGGCCUCCUGGGU | 4010 | ACCCAGGAGGCCUCGAGGG |
| siRNA 1113 | 1113 | CCUCGAGGCCUCCUGGGUG | 4011 | CACCCAGGAGGCCUCGAGG |
| siRNA 1114 | 1114 | CUCGAGGCCUCCUGGGUGG | 4012 | CCACCCAGGAGGCCUCGAG |
| siRNA 1115 | 1115 | UCGAGGCCUCCUGGGUGGU | 4013 | ACCACCCAGGAGGCCUCGA |
| siRNA 1116 | 1116 | CGAGGCCUCCUGGGUGGUG | 4014 | CACCACCCAGGAGGCCUCG |
| siRNA 1117 | 1117 | GAGGCCUCCUGGGUGGUGU | 4015 | ACACCACCCAGGAGGCCUC |
| siRNA 1118 | 1118 | AGGCCUCCUGGGUGGUGUG | 4016 | CACACCACCCAGGAGGCCU |
| siRNA 1119 | 1119 | GGCCUCCUGGGUGGUGUGG | 4017 | CCACACCACCCAGGAGGCC |
| siRNA 1120 | 1120 | GCCUCCUGGGUGGUGUGGC | 4018 | GCCACACCACCCAGGAGGC |
| siRNA 1121 | 1121 | CCUCCUGGGUGGUGUGGCA | 4019 | UGCCACACCACCCAGGAGG |
| siRNA 1122 | 1122 | CUCCUGGGUGGUGUGGCAC | 4020 | GUGCCACACCACCCAGGAG |
| siRNA 1123 | 1123 | UCCUGGGUGGUGUGGCACA | 4021 | UGUGCCACACCACCCAGGA |
| siRNA 1124 | 1124 | CCUGGGUGGUGUGGCACAU | 4022 | AUGUGCCACACCACCCAGG |
| siRNA 1125 | 1125 | CUGGGUGGUGUGGCACAUA | 4023 | UAUGUGCCACACCACCCAG |
| siRNA 1126 | 1126 | UGGGUGGUGUGGCACAUAC | 4024 | GUAUGUGCCACACCACCCA |
| siRNA 1127 | 1127 | GGGUGGUGUGGCACAUACC | 4025 | GGUAUGUGCCACACCACCC |
| siRNA 1128 | 1128 | GGUGGUGUGGCACAUACCC | 4026 | GGGUAUGUGCCACACCACC |
| siRNA 1129 | 1129 | GUGGUGUGGCACAUACCCU | 4027 | AGGGUAUGUGCCACACCAC |
| siRNA 1130 | 1130 | UGGUGUGGCACAUACCCUG | 4028 | CAGGGUAUGUGCCACACCA |
| siRNA 1131 | 1131 | GGUGUGGCACAUACCCUGC | 4029 | GCAGGGUAUGUGCCACACC |
| siRNA 1132 | 1132 | GUGUGGCACAUACCCUGCA | 4030 | UGCAGGGUAUGUGCCACAC |
| siRNA 1133 | 1133 | UGUGGCACAUACCCUGCAG | 4031 | CUGCAGGGUAUGUGCCACA |
| siRNA 1134 | 1134 | GUGGCACAUACCCUGCAGA | 4032 | UCUGCAGGGUAUGUGCCAC |
| siRNA 1135 | 1135 | UGGCACAUACCCUGCAGAA | 4033 | UUCUGCAGGGUAUGUGCCA |
| siRNA 1136 | 1136 | GGCACAUACCCUGCAGAAG | 4034 | CUUCUGCAGGGUAUGUGCC |
| siRNA 1137 | 1137 | GCACAUACCCUGCAGAAGA | 4035 | UCUUCUGCAGGGUAUGUGC |
| SIRNA 1138 | 1138 | CACAUACCCUGCAGAAGAC | 4036 | GUCUUCUGCAGGGUAUGUG |
| siRNA 1139 | 1139 | ACAUACCCUGCAGAAGACC | 4037 | GGUCUUCUGCAGGGUAUGU |
| siRNA 1140 | 1140 | CAUACCCUGCAGAAGACCC | 4038 | GGGUCUUCUGCAGGGUAUG |
| siRNA 1141 | 1141 | AUACCCUGCAGAAGACCCU | 4039 | AGGGUCUUCUGCAGGGUAU |
| siRNA 1142 | 1142 | UACCCUGCAGAAGACCCUC | 4040 | GAGGGUCUUCUGCAGGGUA |
| siRNA 1143 | 1143 | ACCCUGCAGAAGACCCUCC | 4041 | GGAGGGUCUUCUGCAGGGU |
| siRNA 1144 | 1144 | CCCUGCAGAAGACCCUCCA | 4042 | UGGAGGGUCUUCUGCAGGG |
| siRNA 1145 | 1145 | CCUGCAGAAGACCCUCCAG | 4043 | CUGGAGGGUCUUCUGCAGG |
| siRNA 1146 | 1146 | CUGCAGAAGACCCUCCAGA | 4044 | UCUGGAGGGUCUUCUGCAG |
| siRNA 1147 | 1147 | UGCAGAAGACCCUCCAGAC | 4045 | GUCUGGAGGGUCUUCUGCA |
| siRNA 1148 | 1148 | GCAGAAGACCCUCCAGACC | 4046 | GGUCUGGAGGGUCUUCUGC |
| siRNA 1149 | 1149 | CAGAAGACCCUCCAGACCA | 4047 | UGGUCUGGAGGGUCUUCUG |
| siRNA 1150 | 1150 | AGAAGACCCUCCAGACCAC | 4048 | GUGGUCUGGAGGGUCUUCU |
| siRNA 1151 | 1151 | GAAGACCCUCCAGACCACC | 4049 | GGUGGUCUGGAGGGUCUUC |
| siRNA 1152 | 1152 | AAGACCCUCCAGACCACCA | 4050 | UGGUGGUCUGGAGGGUCUU |
| siRNA 1153 | 1153 | AGACCCUCCAGACCACCAU | 4051 | AUGGUGGUCUGGAGGGUCU |
| SiRNA 1154 | 1154 | GACCCUCCAGACCACCAUC | 4052 | GAUGGUGGUCUGGAGGGUC |
| siRNA 1155 | 1155 | ACCCUCCAGACCACCAUCU | 4053 | AGAUGGUGGUCUGGAGGGU |
| siRNA 1156 | 1156 | CCCUCCAGACCACCAUCUC | 4054 | GAGAUGGUGGUCUGGAGGG |
| siRNA 1157 | 1157 | CCUCCAGACCACCAUCUCG | 4055 | CGAGAUGGUGGUCUGGAGG |
| siRNA 1158 | 1158 | CUCCAGACCACCAUCUCGG | 4056 | CCGAGAUGGUGGUCUGGAG |
| siRNA 1159 | 1159 | UCCAGACCACCAUCUCGGC | 4057 | GCCGAGAUGGUGGUCUGGA |
| siRNA 1160 | 1160 | CCAGACCACCAUCUCGGCU | 4058 | AGCCGAGAUGGUGGUCUGG |
| siRNA 1161 | 1161 | CAGACCACCAUCUCGGCUG | 4059 | CAGCCGAGAUGGUGGUCUG |
| SIRNA 1162 | 1162 | AGACCACCAUCUCGGCUGU | 4060 | ACAGCCGAGAUGGUGGUCU |
| siRNA 1163 | 1163 | GACCACCAUCUCGGCUGUG | 4061 | CACAGCCGAGAUGGUGGUC |
| siRNA 1164 | 1164 | ACCACCAUCUCGGCUGUGA | 4062 | UCACAGCCGAGAUGGUGGU |
| siRNA 1165 | 1165 | CCACCAUCUCGGCUGUGAC | 4063 | GUCACAGCCGAGAUGGUGG |
| siRNA 1166 | 1166 | CACCAUCUCGGCUGUGACA | 4064 | UGUCACAGCCGAGAUGGUG |
| siRNA 1167 | 1167 | ACCAUCUCGGCUGUGACAU | 4065 | AUGUCACAGCCGAGAUGGU |
| siRNA 1168 | 1168 | CCAUCUCGGCUGUGACAUG | 4066 | CAUGUCACAGCCGAGAUGG |
| siRNA 1169 | 1169 | CAUCUCGGCUGUGACAUGG | 4067 | CCAUGUCACAGCCGAGAUG |
| siRNA 1170 | 1170 | AUCUCGGCUGUGACAUGGG | 4068 | CCCAUGUCACAGCCGAGAU |
| siRNA 1171 | 1171 | UCUCGGCUGUGACAUGGGC | 4069 | GCCCAUGUCACAGCCGAGA |
| siRNA 1172 | 1172 | CUCGGCUGUGACAUGGGCA | 4070 | UGCCCAUGUCACAGCCGAG |
| siRNA 1173 | 1173 | UCGGCUGUGACAUGGGCAC | 4071 | GUGCCCAUGUCACAGCCGA |
| siRNA 1174 | 1174 | CGGCUGUGACAUGGGCACC | 4072 | GGUGCCCAUGUCACAGCCG |
| siRNA 1175 | 1175 | GGCUGUGACAUGGGCACCU | 4073 | AGGUGCCCAUGUCACAGCC |
| siRNA 1176 | 1176 | GCUGUGACAUGGGCACCUG | 4074 | CAGGUGCCCAUGUCACAGC |
| siRNA 1177 | 1177 | CUGUGACAUGGGCACCUGC | 4075 | GCAGGUGCCCAUGUCACAG |
| siRNA 1178 | 1178 | UGUGACAUGGGCACCUGCA | 4076 | UGCAGGUGCCCAUGUCACA |
| siRNA 1179 | 1179 | GUGACAUGGGCACCUGCAG | 4077 | CUGCAGGUGCCCAUGUCAC |
| siRNA 1180 | 1180 | UGACAUGGGCACCUGCAGC | 4078 | GCUGCAGGUGCCCAUGUCA |
| siRNA 1181 | 1181 | GACAUGGGCACCUGCAGCU | 4079 | AGCUGCAGGUGCCCAUGUC |
| siRNA 1182 | 1182 | ACAUGGGCACCUGCAGCUG | 4080 | CAGCUGCAGGUGCCCAUGU |
| siRNA 1183 | 1183 | CAUGGGCACCUGCAGCUGU | 4081 | ACAGCUGCAGGUGCCCAUG |
| siRNA 1184 | 1184 | AUGGGCACCUGCAGCUGUG | 4082 | CACAGCUGCAGGUGCCCAU |
| siRNA 1185 | 1185 | UGGGCACCUGCAGCUGUGC | 4083 | GCACAGCUGCAGGUGCCCA |
| siRNA 1186 | 1186 | GGGCACCUGCAGCUGUGCU | 4084 | AGCACAGCUGCAGGUGCCC |
| siRNA 1187 | 1187 | GGCACCUGCAGCUGUGCUG | 4085 | CAGCACAGCUGCAGGUGCC |
| siRNA 1188 | 1188 | GCACCUGCAGCUGUGCUGG | 4086 | CCAGCACAGCUGCAGGUGC |
| siRNA 1189 | 1189 | CACCUGCAGCUGUGCUGGG | 4087 | CCCAGCACAGCUGCAGGUG |
| siRNA 1190 | 1190 | ACCUGCAGCUGUGCUGGGC | 4088 | GCCCAGCACAGCUGCAGGU |
| siRNA 1191 | 1191 | CCUGCAGCUGUGCUGGGCA | 4089 | UGCCCAGCACAGCUGCAGG |
| siRNA 1192 | 1192 | CUGCAGCUGUGCUGGGCAU | 4090 | AUGCCCAGCACAGCUGCAG |
| siRNA 1193 | 1193 | UGCAGCUGUGCUGGGCAUG | 4091 | CAUGCCCAGCACAGCUGCA |
| siRNA 1194 | 1194 | GCAGCUGUGCUGGGCAUGG | 4092 | CCAUGCCCAGCACAGCUGC |
| siRNA 1195 | 1195 | CAGCUGUGCUGGGCAUGGC | 4093 | GCCAUGCCCAGCACAGCUG |
| siRNA 1196 | 1196 | AGCUGUGCUGGGCAUGGCA | 4094 | UGCCAUGCCCAGCACAGCU |
| siRNA 1197 | 1197 | GCUGUGCUGGGCAUGGCAG | 4095 | CUGCCAUGCCCAGCACAGC |
| siRNA 1198 | 1198 | CUGUGCUGGGCAUGGCAGG | 4096 | CCUGCCAUGCCCAGCACAG |
| siRNA 1199 | 1199 | UGUGCUGGGCAUGGCAGGG | 4097 | CCCUGCCAUGCCCAGCACA |
| siRNA 1200 | 1200 | GUGCUGGGCAUGGCAGGGA | 4098 | UCCCUGCCAUGCCCAGCAC |
| siRNA 1201 | 1201 | UGCUGGGCAUGGCAGGGAG | 4099 | CUCCCUGCCAUGCCCAGCA |
| SiRNA 1202 | 1202 | GCUGGGCAUGGCAGGGAGG | 4100 | CCUCCCUGCCAUGCCCAGC |
| siRNA 1203 | 1203 | CUGGGCAUGGCAGGGAGGG | 4101 | CCCUCCCUGCCAUGCCCAG |
| siRNA 1204 | 1204 | UGGGCAUGGCAGGGAGGGU | 4102 | ACCCUCCCUGCCAUGCCCA |
| siRNA 1205 | 1205 | GGGCAUGGCAGGGAGGGUG | 4103 | CACCCUCCCUGCCAUGCCC |
| siRNA 1206 | 1206 | GGCAUGGCAGGGAGGGUGC | 4104 | GCACCCUCCCUGCCAUGCC |
| siRNA 1207 | 1207 | GCAUGGCAGGGAGGGUGCU | 4105 | AGCACCCUCCCUGCCAUGC |
| siRNA 1208 | 1208 | CAUGGCAGGGAGGGUGCUG | 4106 | CAGCACCCUCCCUGCCAUG |
| siRNA 1209 | 1209 | AUGGCAGGGAGGGUGCUGC | 4107 | GCAGCACCCUCCCUGCCAU |
| siRNA 1210 | 1210 | UGGCAGGGAGGGUGCUGCA | 4108 | UGCAGCACCCUCCCUGCCA |
| siRNA 1211 | 1211 | GGCAGGGAGGGUGCUGCAC | 4109 | GUGCAGCACCCUCCCUGCC |
| siRNA 1212 | 1212 | GCAGGGAGGGUGCUGCACC | 4110 | GGUGCAGCACCCUCCCUGC |
| siRNA 1213 | 1213 | CAGGGAGGGUGCUGCACCU | 4111 | AGGUGCAGCACCCUCCCUG |
| siRNA 1214 | 1214 | AGGGAGGGUGCUGCACCUC | 4112 | GAGGUGCAGCACCCUCCCU |
| siRNA 1215 | 1215 | GGGAGGGUGCUGCACCUCA | 4113 | UGAGGUGCAGCACCCUCCC |
| siRNA 1216 | 1216 | GGAGGGUGCUGCACCUCAC | 4114 | GUGAGGUGCAGCACCCUCC |
| siRNA 1217 | 1217 | GAGGGUGCUGCACCUCACA | 4115 | UGUGAGGUGCAGCACCCUC |
| siRNA 1218 | 1218 | AGGGUGCUGCACCUCACAC | 4116 | GUGUGAGGUGCAGCACCCU |
| siRNA 1219 | 1219 | GGGUGCUGCACCUCACACC | 4117 | GGUGUGAGGUGCAGCACCC |
| siRNA 1220 | 1220 | GGUGCUGCACCUCACACCA | 4118 | UGGUGUGAGGUGCAGCACC |
| siRNA 1221 | 1221 | GUGCUGCACCUCACACCAG | 4119 | CUGGUGUGAGGUGCAGCAC |
| siRNA 1222 | 1222 | UGCUGCACCUCACACCAGC | 4120 | GCUGGUGUGAGGUGCAGCA |
| siRNA 1223 | 1223 | GCUGCACCUCACACCAGCC | 4121 | GGCUGGUGUGAGGUGCAGC |
| siRNA 1224 | 1224 | CUGCACCUCACACCAGCCC | 4122 | GGGCUGGUGUGAGGUGCAG |
| siRNA 1225 | 1225 | UGCACCUCACACCAGCCCC | 4123 | GGGGCUGGUGUGAGGUGCA |
| siRNA 1226 | 1226 | GCACCUCACACCAGCCCCU | 4124 | AGGGGCUGGUGUGAGGUGC |
| siRNA 1227 | 1227 | CACCUCACACCAGCCCCUG | 4125 | CAGGGGCUGGUGUGAGGUG |
| siRNA 1228 | 1228 | ACCUCACACCAGCCCCUGC | 4126 | GCAGGGGCUGGUGUGAGGU |
| siRNA 1229 | 1229 | CCUCACACCAGCCCCUGCU | 4127 | AGCAGGGGCUGGUGUGAGG |
| siRNA 1230 | 1230 | CUCACACCAGCCCCUGCUG | 4128 | CAGCAGGGGCUGGUGUGAG |
| siRNA 1231 | 1231 | UCACACCAGCCCCUGCUGU | 4129 | ACAGCAGGGGCUGGUGUGA |
| siRNA 1232 | 1232 | CACACCAGCCCCUGCUGUC | 4130 | GACAGCAGGGGCUGGUGUG |
| siRNA 1233 | 1233 | ACACCAGCCCCUGCUGUCU | 4131 | AGACAGCAGGGGCUGGUGU |
| siRNA 1234 | 1234 | CACCAGCCCCUGCUGUCUC | 4132 | GAGACAGCAGGGGCUGGUG |
| siRNA 1235 | 1235 | ACCAGCCCCUGCUGUCUCC | 4133 | GGAGACAGCAGGGGCUGGU |
| siRNA 1236 | 1236 | CCAGCCCCUGCUGUCUCCU | 4134 | AGGAGACAGCAGGGGCUGG |
| siRNA 1237 | 1237 | CAGCCCCUGCUGUCUCCUC | 4135 | GAGGAGACAGCAGGGGCUG |
| siRNA 1238 | 1238 | AGCCCCUGCUGUCUCCUCA | 4136 | UGAGGAGACAGCAGGGGCU |
| siRNA 1239 | 1239 | GCCCCUGCUGUCUCCUCAA | 4137 | UUGAGGAGACAGCAGGGGC |
| SiRNA 1240 | 1240 | CCCCUGCUGUCUCCUCAAC | 4138 | GUUGAGGAGACAGCAGGGG |
| siRNA 1241 | 1241 | CCCUGCUGUCUCCUCAACC | 4139 | GGUUGAGGAGACAGCAGGG |
| siRNA 1242 | 1242 | CCUGCUGUCUCCUCAACCA | 4140 | UGGUUGAGGAGACAGCAGG |
| siRNA 1243 | 1243 | CUGCUGUCUCCUCAACCAA | 4141 | UUGGUUGAGGAGACAGCAG |
| siRNA 1244 | 1244 | UGCUGUCUCCUCAACCAAG | 4142 | CUUGGUUGAGGAGACAGCA |
| siRNA 1245 | 1245 | GCUGUCUCCUCAACCAAGG | 4143 | CCUUGGUUGAGGAGACAGC |
| siRNA 1246 | 1246 | CUGUCUCCUCAACCAAGGG | 4144 | CCCUUGGUUGAGGAGACAG |
| siRNA 1247 | 1247 | UGUCUCCUCAACCAAGGGG | 4145 | CCCCUUGGUUGAGGAGACA |
| siRNA 1248 | 1248 | GUCUCCUCAACCAAGGGGA | 4146 | UCCCCUUGGUUGAGGAGAC |
| siRNA 1249 | 1249 | UCUCCUCAACCAAGGGGAG | 4147 | CUCCCCUUGGUUGAGGAGA |
| siRNA 1250 | 1250 | CUCCUCAACCAAGGGGAGG | 4148 | CCUCCCCUUGGUUGAGGAG |
| siRNA 1251 | 1251 | UCCUCAACCAAGGGGAGGG | 4149 | CCCUCCCCUUGGUUGAGGA |
| siRNA 1252 | 1252 | CCUCAACCAAGGGGAGGGC | 4150 | GCCCUCCCCUUGGUUGAGG |
| siRNA 1253 | 1253 | CUCAACCAAGGGGAGGGCC | 4151 | GGCCCUCCCCUUGGUUGAG |
| siRNA 1254 | 1254 | UCAACCAAGGGGAGGGCCA | 4152 | UGGCCCUCCCCUUGGUUGA |
| siRNA 1255 | 1255 | CAACCAAGGGGAGGGCCAU | 4153 | AUGGCCCUCCCCUUGGUUG |
| siRNA 1256 | 1256 | AACCAAGGGGAGGGCCAUG | 4154 | CAUGGCCCUCCCCUUGGUU |
| siRNA 1257 | 1257 | ACCAAGGGGAGGGCCAUGU | 4155 | ACAUGGCCCUCCCCUUGGU |
| siRNA 1258 | 1258 | CCAAGGGGAGGGCCAUGUC | 4156 | GACAUGGCCCUCCCCUUGG |
| siRNA 1259 | 1259 | CAAGGGGAGGGCCAUGUCC | 4157 | GGACAUGGCCCUCCCCUUG |
| siRNA 1260 | 1260 | AAGGGGAGGGCCAUGUCCC | 4158 | GGGACAUGGCCCUCCCCUU |
| siRNA 1261 | 1261 | AGGGGAGGGCCAUGUCCCU | 4159 | AGGGACAUGGCCCUCCCCU |
| siRNA 1262 | 1262 | GGGGAGGGCCAUGUCCCUA | 4160 | UAGGGACAUGGCCCUCCCC |
| siRNA 1263 | 1263 | GGGAGGGCCAUGUCCCUAU | 4161 | AUAGGGACAUGGCCCUCCC |
| siRNA 1264 | 1264 | GGAGGGCCAUGUCCCUAUC | 4162 | GAUAGGGACAUGGCCCUCC |
| siRNA 1265 | 1265 | GAGGGCCAUGUCCCUAUCA | 4163 | UGAUAGGGACAUGGCCCUC |
| siRNA 1266 | 1266 | AGGGCCAUGUCCCUAUCAG | 4164 | CUGAUAGGGACAUGGCCCU |
| SIRNA 1267 | 1267 | GGGCCAUGUCCCUAUCAGA | 4165 | UCUGAUAGGGACAUGGCCC |
| siRNA 1268 | 1268 | GGCCAUGUCCCUAUCAGAU | 4166 | AUCUGAUAGGGACAUGGCC |
| siRNA 1269 | 1269 | GCCAUGUCCCUAUCAGAUG | 4167 | CAUCUGAUAGGGACAUGGC |
| siRNA 1270 | 1270 | CCAUGUCCCUAUCAGAUGC | 4168 | GCAUCUGAUAGGGACAUGG |
| siRNA 1271 | 1271 | CAUGUCCCUAUCAGAUGCC | 4169 | GGCAUCUGAUAGGGACAUG |
| siRNA 1272 | 1272 | AUGUCCCUAUCAGAUGCCC | 4170 | GGGCAUCUGAUAGGGACAU |
| siRNA 1273 | 1273 | UGUCCCUAUCAGAUGCCCU | 4171 | AGGGCAUCUGAUAGGGACA |
| siRNA 1274 | 1274 | GUCCCUAUCAGAUGCCCUG | 4172 | CAGGGCAUCUGAUAGGGAC |
| siRNA 1275 | 1275 | UCCCUAUCAGAUGCCCUGA | 4173 | UCAGGGCAUCUGAUAGGGA |
| siRNA 1276 | 1276 | CCCUAUCAGAUGCCCUGAA | 4174 | UUCAGGGCAUCUGAUAGGG |
| siRNA 1277 | 1277 | CCUAUCAGAUGCCCUGAAG | 4175 | CUUCAGGGCAUCUGAUAGG |
| siRNA 1278 | 1278 | CUAUCAGAUGCCCUGAAGG | 4176 | CCUUCAGGGCAUCUGAUAG |
| siRNA 1279 | 1279 | UAUCAGAUGCCCUGAAGGG | 4177 | CCCUUCAGGGCAUCUGAUA |
| siRNA 1280 | 1280 | AUCAGAUGCCCUGAAGGGC | 4178 | GCCCUUCAGGGCAUCUGAU |
| siRNA 1281 | 1281 | UCAGAUGCCCUGAAGGGCG | 4179 | CGCCCUUCAGGGCAUCUGA |
| siRNA 1282 | 1282 | CAGAUGCCCUGAAGGGCGU | 4180 | ACGCCCUUCAGGGCAUCUG |
| siRNA 1283 | 1283 | AGAUGCCCUGAAGGGCGUU | 4181 | AACGCCCUUCAGGGCAUCU |
| siRNA 1284 | 1284 | GAUGCCCUGAAGGGCGUUA | 4182 | UAACGCCCUUCAGGGCAUC |
| siRNA 1285 | 1285 | AUGCCCUGAAGGGCGUUAC | 4183 | GUAACGCCCUUCAGGGCAU |
| siRNA 1286 | 1286 | UGCCCUGAAGGGCGUUACU | 4184 | AGUAACGCCCUUCAGGGCA |
| siRNA 1287 | 1287 | GCCCUGAAGGGCGUUACUG | 4185 | CAGUAACGCCCUUCAGGGC |
| siRNA 1288 | 1288 | CCCUGAAGGGCGUUACUGA | 4186 | UCAGUAACGCCCUUCAGGG |
| siRNA 1289 | 1289 | CCUGAAGGGCGUUACUGAC | 4187 | GUCAGUAACGCCCUUCAGG |
| siRNA 1290 | 1290 | CUGAAGGGCGUUACUGACA | 4188 | UGUCAGUAACGCCCUUCAG |
| siRNA 1291 | 1291 | UGAAGGGCGUUACUGACAA | 4189 | UUGUCAGUAACGCCCUUCA |
| siRNA 1292 | 1292 | GAAGGGCGUUACUGACAAC | 4190 | GUUGUCAGUAACGCCCUUC |
| siRNA 1293 | 1293 | AAGGGCGUUACUGACAACG | 4191 | CGUUGUCAGUAACGCCCUU |
| siRNA 1294 | 1294 | AGGGCGUUACUGACAACGU | 4192 | ACGUUGUCAGUAACGCCCU |
| siRNA 1295 | 1295 | GGGCGUUACUGACAACGUG | 4193 | CACGUUGUCAGUAACGCCC |
| siRNA 1296 | 1296 | GGCGUUACUGACAACGUGG | 4194 | CCACGUUGUCAGUAACGCC |
| siRNA 1297 | 1297 | GCGUUACUGACAACGUGGU | 4195 | ACCACGUUGUCAGUAACGC |
| siRNA 1298 | 1298 | CGUUACUGACAACGUGGUG | 4196 | CACCACGUUGUCAGUAACG |
| siRNA 1299 | 1299 | GUUACUGACAACGUGGUGG | 4197 | CCACCACGUUGUCAGUAAC |
| siRNA 1300 | 1300 | UUACUGACAACGUGGUGGA | 4198 | UCCACCACGUUGUCAGUAA |
| siRNA 1301 | 1301 | UACUGACAACGUGGUGGAC | 4199 | GUCCACCACGUUGUCAGUA |
| siRNA 1302 | 1302 | ACUGACAACGUGGUGGACA | 4200 | UGUCCACCACGUUGUCAGU |
| SiRNA 1303 | 1303 | CUGACAACGUGGUGGACAC | 4201 | GUGUCCACCACGUUGUCAG |
| siRNA 1304 | 1304 | UGACAACGUGGUGGACACA | 4202 | UGUGUCCACCACGUUGUCA |
| siRNA 1305 | 1305 | GACAACGUGGUGGACACAG | 4203 | CUGUGUCCACCACGUUGUC |
| siRNA 1306 | 1306 | ACAACGUGGUGGACACAGU | 4204 | ACUGUGUCCACCACGUUGU |
| siRNA 1307 | 1307 | CAACGUGGUGGACACAGUG | 4205 | CACUGUGUCCACCACGUUG |
| siRNA 1308 | 1308 | AACGUGGUGGACACAGUGG | 4206 | CCACUGUGUCCACCACGUU |
| siRNA 1309 | 1309 | ACGUGGUGGACACAGUGGU | 4207 | ACCACUGUGUCCACCACGU |
| siRNA 1310 | 1310 | CGUGGUGGACACAGUGGUG | 4208 | CACCACUGUGUCCACCACG |
| siRNA 1311 | 1311 | GUGGUGGACACAGUGGUGC | 4209 | GCACCACUGUGUCCACCAC |
| siRNA 1312 | 1312 | UGGUGGACACAGUGGUGCA | 4210 | UGCACCACUGUGUCCACCA |
| siRNA 1313 | 1313 | GGUGGACACAGUGGUGCAU | 4211 | AUGCACCACUGUGUCCACC |
| siRNA 1314 | 1314 | GUGGACACAGUGGUGCAUU | 4212 | AAUGCACCACUGUGUCCAC |
| siRNA 1315 | 1315 | UGGACACAGUGGUGCAUUA | 4213 | UAAUGCACCACUGUGUCCA |
| siRNA 1316 | 1316 | GGACACAGUGGUGCAUUAC | 4214 | GUAAUGCACCACUGUGUCC |
| siRNA 1317 | 1317 | GACACAGUGGUGCAUUACG | 4215 | CGUAAUGCACCACUGUGUC |
| siRNA 1318 | 1318 | ACACAGUGGUGCAUUACGU | 4216 | ACGUAAUGCACCACUGUGU |
| siRNA 1319 | 1319 | CACAGUGGUGCAUUACGUG | 4217 | CACGUAAUGCACCACUGUG |
| siRNA 1320 | 1320 | ACAGUGGUGCAUUACGUGC | 4218 | GCACGUAAUGCACCACUGU |
| siRNA 1321 | 1321 | CAGUGGUGCAUUACGUGCC | 4219 | GGCACGUAAUGCACCACUG |
| siRNA 1322 | 1322 | AGUGGUGCAUUACGUGCCG | 4220 | CGGCACGUAAUGCACCACU |
| siRNA 1323 | 1323 | GUGGUGCAUUACGUGCCGC | 4221 | GCGGCACGUAAUGCACCAC |
| siRNA 1324 | 1324 | UGGUGCAUUACGUGCCGCU | 4222 | AGCGGCACGUAAUGCACCA |
| siRNA 1325 | 1325 | GGUGCAUUACGUGCCGCUC | 4223 | GAGCGGCACGUAAUGCACC |
| siRNA 1326 | 1326 | GUGCAUUACGUGCCGCUCC | 4224 | GGAGCGGCACGUAAUGCAC |
| siRNA 1327 | 1327 | UGCAUUACGUGCCGCUCCC | 4225 | GGGAGCGGCACGUAAUGCA |
| siRNA 1328 | 1328 | GCAUUACGUGCCGCUCCCC | 4226 | GGGGAGCGGCACGUAAUGC |
| siRNA 1329 | 1329 | CAUUACGUGCCGCUCCCCA | 4227 | UGGGGAGCGGCACGUAAUG |
| siRNA 1330 | 1330 | AUUACGUGCCGCUCCCCAG | 4228 | CUGGGGAGCGGCACGUAAU |
| siRNA 1331 | 1331 | UUACGUGCCGCUCCCCAGG | 4229 | CCUGGGGAGCGGCACGUAA |
| siRNA 1332 | 1332 | UACGUGCCGCUCCCCAGGC | 4230 | GCCUGGGGAGCGGCACGUA |
| siRNA 1333 | 1333 | ACGUGCCGCUCCCCAGGCU | 4231 | AGCCUGGGGAGCGGCACGU |
| siRNA 1334 | 1334 | CGUGCCGCUCCCCAGGCUG | 4232 | CAGCCUGGGGAGCGGCACG |
| siRNA 1335 | 1335 | GUGCCGCUCCCCAGGCUGU | 4233 | ACAGCCUGGGGAGCGGCAC |
| siRNA 1336 | 1336 | UGCCGCUCCCCAGGCUGUC | 4234 | GACAGCCUGGGGAGCGGCA |
| siRNA 1337 | 1337 | GCCGCUCCCCAGGCUGUCG | 4235 | CGACAGCCUGGGGAGCGGC |
| siRNA 1338 | 1338 | CCGCUCCCCAGGCUGUCGC | 4236 | GCGACAGCCUGGGGAGCGG |
| siRNA 1339 | 1339 | CGCUCCCCAGGCUGUCGCU | 4237 | AGCGACAGCCUGGGGAGCG |
| siRNA 1340 | 1340 | GCUCCCCAGGCUGUCGCUG | 4238 | CAGCGACAGCCUGGGGAGC |
| siRNA 1341 | 1341 | CUCCCCAGGCUGUCGCUGA | 4239 | UCAGCGACAGCCUGGGGAG |
| siRNA 1342 | 1342 | UCCCCAGGCUGUCGCUGAU | 4240 | AUCAGCGACAGCCUGGGGA |
| siRNA 1343 | 1343 | CCCCAGGCUGUCGCUGAUG | 4241 | CAUCAGCGACAGCCUGGGG |
| siRNA 1344 | 1344 | CCCAGGCUGUCGCUGAUGG | 4242 | CCAUCAGCGACAGCCUGGG |
| siRNA 1345 | 1345 | CCAGGCUGUCGCUGAUGGA | 4243 | UCCAUCAGCGACAGCCUGG |
| siRNA 1346 | 1346 | CAGGCUGUCGCUGAUGGAG | 4244 | CUCCAUCAGCGACAGCCUG |
| siRNA 1347 | 1347 | AGGCUGUCGCUGAUGGAGC | 4245 | GCUCCAUCAGCGACAGCCU |
| siRNA 1348 | 1348 | GGCUGUCGCUGAUGGAGCC | 4246 | GGCUCCAUCAGCGACAGCC |
| siRNA 1349 | 1349 | GCUGUCGCUGAUGGAGCCC | 4247 | GGGCUCCAUCAGCGACAGC |
| siRNA 1350 | 1350 | CUGUCGCUGAUGGAGCCCG | 4248 | CGGGCUCCAUCAGCGACAG |
| siRNA 1351 | 1351 | UGUCGCUGAUGGAGCCCGA | 4249 | UCGGGCUCCAUCAGCGACA |
| siRNA 1352 | 1352 | GUCGCUGAUGGAGCCCGAG | 4250 | CUCGGGCUCCAUCAGCGAC |
| siRNA 1353 | 1353 | UCGCUGAUGGAGCCCGAGA | 4251 | UCUCGGGCUCCAUCAGCGA |
| siRNA 1354 | 1354 | CGCUGAUGGAGCCCGAGAG | 4252 | CUCUCGGGCUCCAUCAGCG |
| siRNA 1355 | 1355 | GCUGAUGGAGCCCGAGAGC | 4253 | GCUCUCGGGCUCCAUCAGC |
| siRNA 1356 | 1356 | CUGAUGGAGCCCGAGAGCG | 4254 | CGCUCUCGGGCUCCAUCAG |
| siRNA 1357 | 1357 | UGAUGGAGCCCGAGAGCGA | 4255 | UCGCUCUCGGGCUCCAUCA |
| siRNA 1358 | 1358 | GAUGGAGCCCGAGAGCGAA | 4256 | UUCGCUCUCGGGCUCCAUC |
| siRNA 1359 | 1359 | AUGGAGCCCGAGAGCGAAU | 4257 | AUUCGCUCUCGGGCUCCAU |
| siRNA 1360 | 1360 | UGGAGCCCGAGAGCGAAUU | 4258 | AAUUCGCUCUCGGGCUCCA |
| siRNA 1361 | 1361 | GGAGCCCGAGAGCGAAUUC | 4259 | GAAUUCGCUCUCGGGCUCC |
| siRNA 1362 | 1362 | GAGCCCGAGAGCGAAUUCC | 4260 | GGAAUUCGCUCUCGGGCUC |
| siRNA 1363 | 1363 | AGCCCGAGAGCGAAUUCCG | 4261 | CGGAAUUCGCUCUCGGGCU |
| siRNA 1364 | 1364 | GCCCGAGAGCGAAUUCCGG | 4262 | CCGGAAUUCGCUCUCGGGC |
| siRNA 1365 | 1365 | CCCGAGAGCGAAUUCCGGG | 4263 | CCCGGAAUUCGCUCUCGGG |
| siRNA 1366 | 1366 | CCGAGAGCGAAUUCCGGGA | 4264 | UCCCGGAAUUCGCUCUCGG |
| siRNA 1367 | 1367 | CGAGAGCGAAUUCCGGGAC | 4265 | GUCCCGGAAUUCGCUCUCG |
| siRNA 1368 | 1368 | GAGAGCGAAUUCCGGGACA | 4266 | UGUCCCGGAAUUCGCUCUC |
| siRNA 1369 | 1369 | AGAGCGAAUUCCGGGACAU | 4267 | AUGUCCCGGAAUUCGCUCU |
| siRNA 1370 | 1370 | GAGCGAAUUCCGGGACAUC | 4268 | GAUGUCCCGGAAUUCGCUC |
| siRNA 1371 | 1371 | AGCGAAUUCCGGGACAUCG | 4269 | CGAUGUCCCGGAAUUCGCU |
| siRNA 1372 | 1372 | GCGAAUUCCGGGACAUCGA | 4270 | UCGAUGUCCCGGAAUUCGC |
| siRNA 1373 | 1373 | CGAAUUCCGGGACAUCGAC | 4271 | GUCGAUGUCCCGGAAUUCG |
| siRNA 1374 | 1374 | GAAUUCCGGGACAUCGACA | 4272 | UGUCGAUGUCCCGGAAUUC |
| siRNA 1375 | 1375 | AAUUCCGGGACAUCGACAA | 4273 | UUGUCGAUGUCCCGGAAUU |
| siRNA 376 | 1376 | AUUCCGGGACAUCGACAAC | 4274 | GUUGUCGAUGUCCCGGAAU |
| siRNA 1377 | 1377 | UUCCGGGACAUCGACAACC | 4275 | GGUUGUCGAUGUCCCGGAA |
| siRNA 1378 | 1378 | UCCGGGACAUCGACAACCC | 4276 | GGGUUGUCGAUGUCCCGGA |
| siRNA 1379 | 1379 | CCGGGACAUCGACAACCCA | 4277 | UGGGUUGUCGAUGUCCCGG |
| siRNA 1380 | 1380 | CGGGACAUCGACAACCCAC | 4278 | GUGGGUUGUCGAUGUCCCG |
| siRNA 1381 | 1381 | GGGACAUCGACAACCCACC | 4279 | GGUGGGUUGUCGAUGUCCC |
| siRNA 1382 | 1382 | GGACAUCGACAACCCACCA | 4280 | UGGUGGGUUGUCGAUGUCC |
| siRNA 1383 | 1383 | GACAUCGACAACCCACCAG | 4281 | CUGGUGGGUUGUCGAUGUC |
| siRNA 1384 | 1384 | ACAUCGACAACCCACCAGC | 4282 | GCUGGUGGGUUGUCGAUGU |
| siRNA 1385 | 1385 | CAUCGACAACCCACCAGCC | 4283 | GGCUGGUGGGUUGUCGAUG |
| siRNA 1386 | 1386 | AUCGACAACCCACCAGCCG | 4284 | CGGCUGGUGGGUUGUCGAU |
| siRNA 1387 | 1387 | UCGACAACCCACCAGCCGA | 4285 | UCGGCUGGUGGGUUGUCGA |
| siRNA 1388 | 1388 | CGACAACCCACCAGCCGAG | 4286 | CUCGGCUGGUGGGUUGUCG |
| siRNA 1389 | 1389 | GACAACCCACCAGCCGAGG | 4287 | CCUCGGCUGGUGGGUUGUC |
| siRNA 1390 | 1390 | ACAACCCACCAGCCGAGGU | 4288 | ACCUCGGCUGGUGGGUUGU |
| siRNA 1391 | 1391 | CAACCCACCAGCCGAGGUC | 4289 | GACCUCGGCUGGUGGGUUG |
| siRNA 1392 | 1392 | AACCCACCAGCCGAGGUCG | 4290 | CGACCUCGGCUGGUGGGUU |
| siRNA 1393 | 1393 | ACCCACCAGCCGAGGUCGA | 4291 | UCGACCUCGGCUGGUGGGU |
| siRNA 1394 | 1394 | CCCACCAGCCGAGGUCGAG | 4292 | CUCGACCUCGGCUGGUGGG |
| siRNA 1395 | 1395 | CCACCAGCCGAGGUCGAGC | 4293 | GCUCGACCUCGGCUGGUGG |
| siRNA 1396 | 1396 | CACCAGCCGAGGUCGAGCG | 4294 | CGCUCGACCUCGGCUGGUG |
| siRNA 1397 | 1397 | ACCAGCCGAGGUCGAGCGC | 4295 | GCGCUCGACCUCGGCUGGU |
| siRNA 1398 | 1398 | CCAGCCGAGGUCGAGCGCC | 4296 | GGCGCUCGACCUCGGCUGG |
| siRNA 1399 | 1399 | CAGCCGAGGUCGAGCGCCG | 4297 | CGGCGCUCGACCUCGGCUG |
| siRNA 1400 | 1400 | AGCCGAGGUCGAGCGCCGG | 4298 | CCGGCGCUCGACCUCGGCU |
| siRNA 1401 | 1401 | GCCGAGGUCGAGCGCCGGG | 4299 | CCCGGCGCUCGACCUCGGC |
| siRNA 1402 | 1402 | CCGAGGUCGAGCGCCGGGA | 4300 | UCCCGGCGCUCGACCUCGG |
| siRNA 1403 | 1403 | CGAGGUCGAGCGCCGGGAG | 4301 | CUCCCGGCGCUCGACCUCG |
| siRNA 1404 | 1404 | GAGGUCGAGCGCCGGGAGG | 4302 | CCUCCCGGCGCUCGACCUC |
| siRNA 1405 | 1405 | AGGUCGAGCGCCGGGAGGC | 4303 | GCCUCCCGGCGCUCGACCU |
| siRNA 1406 | 1406 | GGUCGAGCGCCGGGAGGCG | 4304 | CGCCUCCCGGCGCUCGACC |
| siRNA 1407 | 1407 | GUCGAGCGCCGGGAGGCGG | 4305 | CCGCCUCCCGGCGCUCGAC |
| siRNA 1408 | 1408 | UCGAGCGCCGGGAGGCGGA | 4306 | UCCGCCUCCCGGCGCUCGA |
| siRNA 1409 | 1409 | CGAGCGCCGGGAGGCGGAG | 4307 | CUCCGCCUCCCGGCGCUCG |
| siRNA 1410 | 1410 | GAGCGCCGGGAGGCGGAGC | 4308 | GCUCCGCCUCCCGGCGCUC |
| siRNA 1411 | 1411 | AGCGCCGGGAGGCGGAGCG | 4309 | CGCUCCGCCUCCCGGCGCU |
| siRNA 1412 | 1412 | GCGCCGGGAGGCGGAGCGC | 4310 | GCGCUCCGCCUCCCGGCGC |
| siRNA 1413 | 1413 | CGCCGGGAGGCGGAGCGCA | 4311 | UGCGCUCCGCCUCCCGGCG |
| siRNA 1414 | 1414 | GCCGGGAGGCGGAGCGCAG | 4312 | CUGCGCUCCGCCUCCCGGC |
| siRNA 1415 | 1415 | CCGGGAGGCGGAGCGCAGA | 4313 | UCUGCGCUCCGCCUCCCGG |
| siRNA 1416 | 1416 | CGGGAGGCGGAGCGCAGAG | 4314 | CUCUGCGCUCCGCCUCCCG |
| siRNA 1417 | 1417 | GGGAGGCGGAGCGCAGAGC | 4315 | GCUCUGCGCUCCGCCUCCC |
| siRNA 1418 | 1418 | GGAGGCGGAGCGCAGAGCG | 4316 | CGCUCUGCGCUCCGCCUCC |
| siRNA 1419 | 1419 | GAGGCGGAGCGCAGAGCGU | 4317 | ACGCUCUGCGCUCCGCCUC |
| siRNA 1420 | 1420 | AGGCGGAGCGCAGAGCGUC | 4318 | GACGCUCUGCGCUCCGCCU |
| siRNA 1421 | 1421 | GGCGGAGCGCAGAGCGUCU | 4319 | AGACGCUCUGCGCUCCGCC |
| siRNA 1422 | 1422 | GCGGAGCGCAGAGCGUCUG | 4320 | CAGACGCUCUGCGCUCCGC |
| siRNA 1423 | 1423 | CGGAGCGCAGAGCGUCUGG | 4321 | CCAGACGCUCUGCGCUCCG |
| siRNA 1424 | 1424 | GGAGCGCAGAGCGUCUGGG | 4322 | CCCAGACGCUCUGCGCUCC |
| siRNA 1425 | 1425 | GAGCGCAGAGCGUCUGGGG | 4323 | CCCCAGACGCUCUGCGCUC |
| siRNA 1426 | 1426 | AGCGCAGAGCGUCUGGGGC | 4324 | GCCCCAGACGCUCUGCGCU |
| siRNA 1427 | 1427 | GCGCAGAGCGUCUGGGGCG | 4325 | CGCCCCAGACGCUCUGCGC |
| siRNA 1428 | 1428 | CGCAGAGCGUCUGGGGCGC | 4326 | GCGCCCCAGACGCUCUGCG |
| siRNA 1429 | 1429 | GCAGAGCGUCUGGGGCGCC | 4327 | GGCGCCCCAGACGCUCUGC |
| siRNA 1430 | 1430 | CAGAGCGUCUGGGGCGCCG | 4328 | CGGCGCCCCAGACGCUCUG |
| siRNA 1431 | 1431 | AGAGCGUCUGGGGCGCCGU | 4329 | ACGGCGCCCCAGACGCUCU |
| siRNA 1432 | 1432 | GAGCGUCUGGGGCGCCGUC | 4330 | GACGGCGCCCCAGACGCUC |
| siRNA 1433 | 1433 | AGCGUCUGGGGCGCCGUCC | 4331 | GGACGGCGCCCCAGACGCU |
| siRNA 1434 | 1434 | GCGUCUGGGGCGCCGUCCG | 4332 | CGGACGGCGCCCCAGACGC |
| siRNA 1435 | 1435 | CGUCUGGGGCGCCGUCCGC | 4333 | GCGGACGGCGCCCCAGACG |
| siRNA 1436 | 1436 | GUCUGGGGCGCCGUCCGCC | 4334 | GGCGGACGGCGCCCCAGAC |
| siRNA 1437 | 1437 | UCUGGGGCGCCGUCCGCCG | 4335 | CGGCGGACGGCGCCCCAGA |
| siRNA 1438 | 1438 | CUGGGGCGCCGUCCGCCGG | 4336 | CCGGCGGACGGCGCCCCAG |
| siRNA 1439 | 1439 | UGGGGCGCCGUCCGCCGGC | 4337 | GCCGGCGGACGGCGCCCCA |
| siRNA 1440 | 1440 | GGGGCGCCGUCCGCCGGCC | 4338 | GGCCGGCGGACGGCGCCCC |
| siRNA 1441 | 1441 | GGGCGCCGUCCGCCGGCCC | 4339 | GGGCCGGCGGACGGCGCCC |
| siRNA 1442 | 1442 | GGCGCCGUCCGCCGGCCCG | 4340 | CGGGCCGGCGGACGGCGCC |
| siRNA 1443 | 1443 | GCGCCGUCCGCCGGCCCGG | 4341 | CCGGGCCGGCGGACGGCGC |
| siRNA 1444 | 1444 | CGCCGUCCGCCGGCCCGGA | 4342 | UCCGGGCCGGCGGACGGCG |
| siRNA 1445 | 1445 | GCCGUCCGCCGGCCCGGAG | 4343 | CUCCGGGCCGGCGGACGGC |
| siRNA 1446 | 1446 | CCGUCCGCCGGCCCGGAGC | 4344 | GCUCCGGGCCGGCGGACGG |
| siRNA 1447 | 1447 | CGUCCGCCGGCCCGGAGCC | 4345 | GGCUCCGGGCCGGCGGACG |
| siRNA 1448 | 1448 | GUCCGCCGGCCCGGAGCCC | 4346 | GGGCUCCGGGCCGGCGGAC |
| siRNA 1449 | 1449 | UCCGCCGGCCCGGAGCCCG | 4347 | CGGGCUCCGGGCCGGCGGA |
| siRNA 1450 | 1450 | CCGCCGGCCCGGAGCCCGC | 4348 | GCGGGCUCCGGGCCGGCGG |
| siRNA 1451 | 1451 | CGCCGGCCCGGAGCCCGCC | 4349 | GGCGGGCUCCGGGCCGGCG |
| siRNA 1452 | 1452 | GCCGGCCCGGAGCCCGCCC | 4350 | GGGCGGGCUCCGGGCCGGC |
| siRNA 1453 | 1453 | CCGGCCCGGAGCCCGCCCC | 4351 | GGGGCGGGCUCCGGGCCGG |
| siRNA 1454 | 1454 | CGGCCCGGAGCCCGCCCCG | 4352 | CGGGGGGGGCUCCGGGCCG |
| siRNA 1455 | 1455 | GGCCCGGAGCCCGCCCCGC | 4353 | GCGGGGGGGGCUCCGGGCC |
| siRNA 1456 | 1456 | GCCCGGAGCCCGCCCCGCG | 4354 | CGCGGGGGGGGCUCCGGGC |
| siRNA 1457 | 1457 | CCCGGAGCCCGCCCCGCGU | 4355 | ACGCGGGGGGGGCUCCGGG |
| siRNA 1458 | 1458 | CCGGAGCCCGCCCCGCGUC | 4356 | GACGCGGGGGGGGCUCCGG |
| siRNA 1459 | 1459 | CGGAGCCCGCCCCGCGUCU | 4357 | AGACGCGGGGGGGGCUCCG |
| siRNA 1460 | 1460 | GGAGCCCGCCCCGCGUCUC | 4358 | GAGACGCGGGGGGGGCUCC |
| siRNA 1461 | 1461 | GAGCCCGCCCCGCGUCUCG | 4359 | CGAGACGCGGGGGGGGCUC |
| siRNA 1462 | 1462 | AGCCCGCCCCGCGUCUCGC | 4360 | GCGAGACGCGGGGCGGGCU |
| siRNA 1463 | 1463 | GCCCGCCCCGCGUCUCGCA | 4361 | UGCGAGACGCGGGGGGGGC |
| siRNA 1464 | 1464 | CCCGCCCCGCGUCUCGCAC | 4362 | GUGCGAGACGCGGGGCGGG |
| siRNA 1465 | 1465 | CCGCCCCGCGUCUCGCACA | 4363 | UGUGCGAGACGCGGGGCGG |
| siRNA 1466 | 1466 | CGCCCCGCGUCUCGCACAG | 4364 | CUGUGCGAGACGCGGGGCG |
| siRNA 1467 | 1467 | GCCCCGCGUCUCGCACAGC | 4365 | GCUGUGCGAGACGCGGGGC |
| siRNA 1468 | 1468 | CCCCGCGUCUCGCACAGCC | 4366 | GGCUGUGCGAGACGCGGGG |
| siRNA 1469 | 1469 | CCCGCGUCUCGCACAGCCC | 4367 | GGGCUGUGCGAGACGCGGG |
| siRNA 1470 | 1470 | CCGCGUCUCGCACAGCCCC | 4368 | GGGGCUGUGCGAGACGCGG |
| siRNA 1471 | 1471 | CGCGUCUCGCACAGCCCCG | 4369 | CGGGGCUGUGCGAGACGCG |
| siRNA 1472 | 1472 | GCGUCUCGCACAGCCCCGC | 4370 | GCGGGGCUGUGCGAGACGC |
| siRNA 1473 | 1473 | CGUCUCGCACAGCCCCGCC | 4371 | GGCGGGGCUGUGCGAGACG |
| siRNA 1474 | 1474 | GUCUCGCACAGCCCCGCCG | 4372 | CGGCGGGGCUGUGCGAGAC |
| siRNA 1475 | 1475 | UCUCGCACAGCCCCGCCGC | 4373 | GCGGCGGGGCUGUGCGAGA |
| siRNA 1476 | 1476 | CUCGCACAGCCCCGCCGCA | 4374 | UGCGGCGGGGCUGUGCGAG |
| siRNA 1477 | 1477 | UCGCACAGCCCCGCCGCAG | 4375 | CUGCGGCGGGGCUGUGCGA |
| siRNA 1478 | 1478 | CGCACAGCCCCGCCGCAGC | 4376 | GCUGCGGCGGGGCUGUGCG |
| siRNA 1479 | 1479 | GCACAGCCCCGCCGCAGCC | 4377 | GGCUGCGGCGGGGCUGUGC |
| siRNA 1480 | 1480 | CACAGCCCCGCCGCAGCCU | 4378 | AGGCUGCGGCGGGGCUGUG |
| siRNA 1481 | 1481 | ACAGCCCCGCCGCAGCCUG | 4379 | CAGGCUGCGGCGGGGCUGU |
| siRNA 1482 | 1482 | CAGCCCCGCCGCAGCCUGC | 4380 | GCAGGCUGCGGCGGGGCUG |
| siRNA 1483 | 1483 | AGCCCCGCCGCAGCCUGCG | 4381 | CGCAGGCUGCGGCGGGGCU |
| siRNA 1484 | 1484 | GCCCCGCCGCAGCCUGCGC | 4382 | GCGCAGGCUGCGGCGGGGC |
| siRNA 1485 | 1485 | CCCCGCCGCAGCCUGCGCA | 4383 | UGCGCAGGCUGCGGCGGGG |
| siRNA 1486 | 1486 | CCCGCCGCAGCCUGCGCAG | 4384 | CUGCGCAGGCUGCGGCGGG |
| siRNA 1487 | 1487 | CCGCCGCAGCCUGCGCAGC | 4385 | GCUGCGCAGGCUGCGGCGG |
| siRNA 1488 | 1488 | CGCCGCAGCCUGCGCAGCG | 4386 | CGCUGCGCAGGCUGCGGCG |
| siRNA 1489 | 1489 | GCCGCAGCCUGCGCAGCGC | 4387 | GCGCUGCGCAGGCUGCGGC |
| siRNA 1490 | 1490 | CCGCAGCCUGCGCAGCGCG | 4388 | CGCGCUGCGCAGGCUGCGG |
| siRNA 1491 | 1491 | CGCAGCCUGCGCAGCGCGC | 4389 | GCGCGCUGCGCAGGCUGCG |
| siRNA 1492 | 1492 | GCAGCCUGCGCAGCGCGCA | 4390 | UGCGCGCUGCGCAGGCUGC |
| siRNA 1493 | 1493 | CAGCCUGCGCAGCGCGCAG | 4391 | CUGCGCGCUGCGCAGGCUG |
| siRNA 1494 | 1494 | AGCCUGCGCAGCGCGCAGA | 4392 | UCUGCGCGCUGCGCAGGCU |
| siRNA 1495 | 1495 | GCCUGCGCAGCGCGCAGAG | 4393 | CUCUGCGCGCUGCGCAGGC |
| siRNA 1496 | 1496 | CCUGCGCAGCGCGCAGAGC | 4394 | GCUCUGCGCGCUGCGCAGG |
| siRNA 1497 | 1497 | CUGCGCAGCGCGCAGAGCC | 4395 | GGCUCUGCGCGCUGCGCAG |
| siRNA 1498 | 1498 | UGCGCAGCGCGCAGAGCCC | 4396 | GGGCUCUGCGCGCUGCGCA |
| siRNA 1499 | 1499 | GCGCAGCGCGCAGAGCCCC | 4397 | GGGGCUCUGCGCGCUGCGC |
| siRNA 1500 | 1500 | CGCAGCGCGCAGAGCCCCG | 4398 | CGGGGCUCUGCGCGCUGCG |
| siRNA 1501 | 1501 | GCAGCGCGCAGAGCCCCGG | 4399 | CCGGGGCUCUGCGCGCUGC |
| siRNA 1502 | 1502 | CAGCGCGCAGAGCCCCGGC | 4400 | GCCGGGGCUCUGCGCGCUG |
| siRNA 1503 | 1503 | AGCGCGCAGAGCCCCGGCG | 4401 | CGCCGGGGCUCUGCGCGCU |
| SiRNA 1504 | 1504 | GCGCGCAGAGCCCCGGCGC | 4402 | GCGCCGGGGCUCUGCGCGC |
| siRNA 1505 | 1505 | CGCGCAGAGCCCCGGCGCG | 4403 | CGCGCCGGGGCUCUGCGCG |
| siRNA 1506 | 1506 | GCGCAGAGCCCCGGCGCGC | 4404 | GCGCGCCGGGGCUCUGCGC |
| siRNA 1507 | 1507 | CGCAGAGCCCCGGCGCGCC | 4405 | GGCGCGCCGGGGCUCUGCG |
| siRNA 1508 | 1508 | GCAGAGCCCCGGCGCGCCC | 4406 | GGGCGCGCCGGGGCUCUGC |
| siRNA 1509 | 1509 | CAGAGCCCCGGCGCGCCCC | 4407 | GGGGCGCGCCGGGGCUCUG |
| siRNA 1510 | 1510 | AGAGCCCCGGCGCGCCCCC | 4408 | GGGGGCGCGCCGGGGCUCU |
| siRNA 1511 | 1511 | GAGCCCCGGCGCGCCCCCC | 4409 | GGGGGGCGCGCCGGGGCUC |
| siRNA 1512 | 1512 | AGCCCCGGCGCGCCCCCCG | 4410 | CGGGGGGCGCGCCGGGGCU |
| siRNA 1513 | 1513 | GCCCCGGCGCGCCCCCGG | 4411 | CCGGGGGGCGCGCCGGGGC |
| siRNA 1514 | 1514 | CCCCGGCGCGCCCCCCGGC | 4412 | GCCGGGGGGCGCGCCGGGG |
| siRNA 1515 | 1515 | CCCGGCGCGCCCCCCGGCC | 4413 | GGCCGGGGGGCGCGCCGGG |
| siRNA 1516 | 1516 | CCGGCGCGCCCCCCGGCCC | 4414 | GGGCCGGGGGGCGCGCCGG |
| siRNA 1517 | 1517 | CGGCGCGCCCCCCGGCCCG | 4415 | CGGGCCGGGGGGCGCGCCG |
| siRNA 1518 | 1518 | GGCGCGCCCCCCGGCCCGG | 4416 | CCGGGCCGGGGGGCGCGCC |
| siRNA 1519 | 1519 | GCGCGCCCCCCGGCCCGGG | 4417 | CCCGGGCCGGGGGGCGCGC |
| siRNA 1520 | 1520 | CGCGCCCCCCGGCCCGGGC | 4418 | GCCCGGGCCGGGGGGCGCG |
| siRNA 1521 | 1521 | GCGCCCCCCGGCCCGGGCC | 4419 | GGCCCGGGCCGGGGGGCGC |
| siRNA 1522 | 1522 | CGCCCCCCGGCCCGGGCCU | 4420 | AGGCCCGGGCCGGGGGGCG |
| siRNA 1523 | 1523 | GCCCCCCGGCCCGGGCCUG | 4421 | CAGGCCCGGGCCGGGGGGC |
| siRNA 1524 | 1524 | CCCCCCGGCCCGGGCCUGG | 4422 | CCAGGCCCGGGCCGGGGGG |
| siRNA 1525 | 1525 | CCCCCGGCCCGGGCCUGGA | 4423 | UCCAGGCCCGGGCCGGGGG |
| siRNA 1526 | 1526 | CCCCGGCCCGGGCCUGGAG | 4424 | CUCCAGGCCCGGGCCGGGG |
| siRNA 1527 | 1527 | CCCGGCCCGGGCCUGGAGG | 4425 | CCUCCAGGCCCGGGCCGGG |
| siRNA 1528 | 1528 | CCGGCCCGGGCCUGGAGGA | 4426 | UCCUCCAGGCCCGGGCCGG |
| siRNA 1529 | 1529 | CGGCCCGGGCCUGGAGGAC | 4427 | GUCCUCCAGGCCCGGGCCG |
| siRNA 1530 | 1530 | GGCCCGGGCCUGGAGGACG | 4428 | CGUCCUCCAGGCCCGGGCC |
| siRNA 1531 | 1531 | GCCCGGGCCUGGAGGACGA | 4429 | UCGUCCUCCAGGCCCGGGC |
| siRNA 1532 | 1532 | CCCGGGCCUGGAGGACGAA | 4430 | UUCGUCCUCCAGGCCCGGG |
| siRNA 1533 | 1533 | CCGGGCCUGGAGGACGAAG | 4431 | CUUCGUCCUCCAGGCCCGG |
| siRNA 1534 | 1534 | CGGGCCUGGAGGACGAAGU | 4432 | ACUUCGUCCUCCAGGCCCG |
| siRNA 1535 | 1535 | GGGCCUGGAGGACGAAGUC | 4433 | GACUUCGUCCUCCAGGCCC |
| siRNA 1536 | 1536 | GGCCUGGAGGACGAAGUCG | 4434 | CGACUUCGUCCUCCAGGCC |
| siRNA 1537 | 1537 | GCCUGGAGGACGAAGUCGC | 4435 | GCGACUUCGUCCUCCAGGC |
| siRNA 1538 | 1538 | CCUGGAGGACGAAGUCGCC | 4436 | GGCGACUUCGUCCUCCAGG |
| siRNA 1539 | 1539 | CUGGAGGACGAAGUCGCCA | 4437 | UGGCGACUUCGUCCUCCAG |
| siRNA 1540 | 1540 | UGGAGGACGAAGUCGCCAC | 4438 | GUGGCGACUUCGUCCUCCA |
| siRNA 1541 | 1541 | GGAGGACGAAGUCGCCACG | 4439 | CGUGGCGACUUCGUCCUCC |
| siRNA 1542 | 1542 | GAGGACGAAGUCGCCACGC | 4440 | GCGUGGCGACUUCGUCCUC |
| siRNA 1543 | 1543 | AGGACGAAGUCGCCACGCC | 4441 | GGCGUGGCGACUUCGUCCU |
| siRNA 1544 | 1544 | GGACGAAGUCGCCACGCCC | 4442 | GGGCGUGGCGACUUCGUCC |
| siRNA 1545 | 1545 | GACGAAGUCGCCACGCCCG | 4443 | CGGGCGUGGCGACUUCGUC |
| siRNA 1546 | 1546 | ACGAAGUCGCCACGCCCGC | 4444 | GCGGGCGUGGCGACUUCGU |
| siRNA 1547 | 1547 | CGAAGUCGCCACGCCCGCA | 4445 | UGCGGGCGUGGCGACUUCG |
| siRNA 1548 | 1548 | GAAGUCGCCACGCCCGCAG | 4446 | CUGCGGGCGUGGCGACUUC |
| siRNA 1549 | 1549 | AAGUCGCCACGCCCGCAGC | 4447 | GCUGCGGGCGUGGCGACUU |
| siRNA 1550 | 1550 | AGUCGCCACGCCCGCAGCG | 4448 | CGCUGCGGGCGUGGCGACU |
| siRNA 1551 | 1551 | GUCGCCACGCCCGCAGCGC | 4449 | GCGCUGCGGGCGUGGCGAC |
| siRNA 1552 | 1552 | UCGCCACGCCCGCAGCGCC | 4450 | GGCGCUGCGGGCGUGGCGA |
| siRNA 1553 | 1553 | CGCCACGCCCGCAGCGCCG | 4451 | CGGCGCUGCGGGCGUGGCG |
| siRNA 1554 | 1554 | GCCACGCCCGCAGCGCCGC | 4452 | GCGGCGCUGCGGGCGUGGC |
| siRNA 1555 | 1555 | CCACGCCCGCAGCGCCGCG | 4453 | CGCGGCGCUGCGGGCGUGG |
| siRNA 1556 | 1556 | CACGCCCGCAGCGCCGCGC | 4454 | GCGCGGCGCUGCGGGCGUG |
| siRNA 1557 | 1557 | ACGCCCGCAGCGCCGCGCC | 4455 | GGCGCGGCGCUGCGGGCGU |
| siRNA 1558 | 1558 | CGCCCGCAGCGCCGCGCCC | 4456 | GGGCGCGGCGCUGCGGGCG |
| siRNA 1559 | 1559 | GCCCGCAGCGCCGCGCCCG | 4457 | CGGGCGCGGCGCUGCGGGC |
| SIRNA 1560 | 1560 | CCCGCAGCGCCGCGCCCGG | 4458 | CCGGGCGCGGCGCUGCGGG |
| siRNA 1561 | 1561 | CCGCAGCGCCGCGCCCGGG | 4459 | CCCGGGCGCGGCGCUGCGG |
| siRNA 1562 | 1562 | CGCAGCGCCGCGCCCGGGC | 4460 | GCCCGGGCGCGGCGCUGCG |
| siRNA 1563 | 1563 | GCAGCGCCGCGCCCGGGCU | 4461 | AGCCCGGGCGCGGCGCUGC |
| siRNA 1564 | 1564 | CAGCGCCGCGCCCGGGCUU | 4462 | AAGCCCGGGCGCGGCGCUG |
| siRNA 1565 | 1565 | AGCGCCGCGCCCGGGCUUC | 4463 | GAAGCCCGGGCGCGGCGCU |
| siRNA 1566 | 1566 | GCGCCGCGCCCGGGCUUCC | 4464 | GGAAGCCCGGGCGCGGCGC |
| siRNA 1567 | 1567 | CGCCGCGCCCGGGCUUCCC | 4465 | GGGAAGCCCGGGCGCGGCG |
| siRNA 1568 | 1568 | GCCGCGCCCGGGCUUCCCG | 4466 | CGGGAAGCCCGGGCGCGGC |
| siRNA 1569 | 1569 | CCGCGCCCGGGCUUCCCGG | 4467 | CCGGGAAGCCCGGGCGCGG |
| siRNA 1570 | 1570 | CGCGCCCGGGCUUCCCGGC | 4468 | GCCGGGAAGCCCGGGCGCG |
| siRNA 1571 | 1571 | GCGCCCGGGCUUCCCGGCC | 4469 | GGCCGGGAAGCCCGGGCGC |
| siRNA 1572 | 1572 | CGCCCGGGCUUCCCGGCCG | 4470 | CGGCCGGGAAGCCCGGGCG |
| siRNA 1573 | 1573 | GCCCGGGCUUCCCGGCCGU | 4471 | ACGGCCGGGAAGCCCGGGC |
| SIRNA 1574 | 1574 | CCCGGGCUUCCCGGCCGUG | 4472 | CACGGCCGGGAAGCCCGGG |
| siRNA 1575 | 1575 | CCGGGCUUCCCGGCCGUGC | 4473 | GCACGGCCGGGAAGCCCGG |
| siRNA 1576 | 1576 | CGGGCUUCCCGGCCGUGCC | 4474 | GGCACGGCCGGGAAGCCCG |
| siRNA 1577 | 1577 | GGGCUUCCCGGCCGUGCCC | 4475 | GGGCACGGCCGGGAAGCCC |
| siRNA 1578 | 1578 | GGCUUCCCGGCCGUGCCCC | 4476 | GGGGCACGGCCGGGAAGCC |
| siRNA 1579 | 1579 | GCUUCCCGGCCGUGCCCCG | 4477 | CGGGGCACGGCCGGGAAGC |
| siRNA 1580 | 1580 | CUUCCCGGCCGUGCCCCGC | 4478 | GCGGGGCACGGCCGGGAAG |
| siRNA 1581 | 1581 | UUCCCGGCCGUGCCCCGCG | 4479 | CGCGGGGCACGGCCGGGAA |
| siRNA 1582 | 1582 | UCCCGGCCGUGCCCCGCGA | 4480 | UCGCGGGGCACGGCCGGGA |
| siRNA 1583 | 1583 | CCCGGCCGUGCCCCGCGAG | 4481 | CUCGCGGGGCACGGCCGGG |
| siRNA 1584 | 1584 | CCGGCCGUGCCCCGCGAGA | 4482 | UCUCGCGGGGCACGGCCGG |
| siRNA 1585 | 1585 | CGGCCGUGCCCCGCGAGAA | 4483 | UUCUCGCGGGGCACGGCCG |
| siRNA 1586 | 1586 | GGCCGUGCCCCGCGAGAAG | 4484 | CUUCUCGCGGGGCACGGCC |
| siRNA 1587 | 1587 | GCCGUGCCCCGCGAGAAGC | 4485 | GCUUCUCGCGGGGCACGGC |
| siRNA 1588 | 1588 | CCGUGCCCCGCGAGAAGCC | 4486 | GGCUUCUCGCGGGGCACGG |
| siRNA 1589 | 1589 | CGUGCCCCGCGAGAAGCCA | 4487 | UGGCUUCUCGCGGGGCACG |
| siRNA 1590 | 1590 | GUGCCCCGCGAGAAGCCAA | 4488 | UUGGCUUCUCGCGGGGCAC |
| siRNA 1591 | 1591 | UGCCCCGCGAGAAGCCAAA | 4489 | UUUGGCUUCUCGCGGGGCA |
| siRNA 1592 | 1592 | GCCCCGCGAGAAGCCAAAG | 4490 | CUUUGGCUUCUCGCGGGGC |
| siRNA 1593 | 1593 | CCCCGCGAGAAGCCAAAGC | 4491 | GCUUUGGCUUCUCGCGGGG |
| siRNA 1594 | 1594 | CCCGCGAGAAGCCAAAGCG | 4492 | CGCUUUGGCUUCUCGCGGG |
| siRNA 1595 | 1595 | CCGCGAGAAGCCAAAGCGC | 4493 | GCGCUUUGGCUUCUCGCGG |
| siRNA 1596 | 1596 | CGCGAGAAGCCAAAGCGCA | 4494 | UGCGCUUUGGCUUCUCGCG |
| siRNA 1597 | 1597 | GCGAGAAGCCAAAGCGCAG | 4495 | CUGCGCUUUGGCUUCUCGC |
| siRNA 1598 | 1598 | CGAGAAGCCAAAGCGCAGG | 4496 | CCUGCGCUUUGGCUUCUCG |
| siRNA 1599 | 1599 | GAGAAGCCAAAGCGCAGGG | 4497 | CCCUGCGCUUUGGCUUCUC |
| siRNA 1600 | 1600 | AGAAGCCAAAGCGCAGGGU | 4498 | ACCCUGCGCUUUGGCUUCU |
| siRNA 1601 | 1601 | GAAGCCAAAGCGCAGGGUC | 4499 | GACCCUGCGCUUUGGCUUC |
| siRNA 1602 | 1602 | AAGCCAAAGCGCAGGGUCA | 4500 | UGACCCUGCGCUUUGGCUU |
| siRNA 1603 | 1603 | AGCCAAAGCGCAGGGUCAG | 4501 | CUGACCCUGCGCUUUGGCU |
| siRNA 1604 | 1604 | GCCAAAGCGCAGGGUCAGC | 4502 | GCUGACCCUGCGCUUUGGC |
| siRNA 1605 | 1605 | CCAAAGCGCAGGGUCAGCG | 4503 | CGCUGACCCUGCGCUUUGG |
| siRNA 1606 | 1606 | CAAAGCGCAGGGUCAGCGA | 4504 | UCGCUGACCCUGCGCUUUG |
| siRNA 1607 | 1607 | AAAGCGCAGGGUCAGCGAC | 4505 | GUCGCUGACCCUGCGCUUU |
| siRNA 1608 | 1608 | AAGCGCAGGGUCAGCGACA | 4506 | UGUCGCUGACCCUGCGCUU |
| siRNA 1609 | 1609 | AGCGCAGGGUCAGCGACAG | 4507 | CUGUCGCUGACCCUGCGCU |
| siRNA 1610 | 1610 | GCGCAGGGUCAGCGACAGC | 4508 | GCUGUCGCUGACCCUGCGC |
| siRNA 1611 | 1611 | CGCAGGGUCAGCGACAGCU | 4509 | AGCUGUCGCUGACCCUGCG |
| siRNA 1612 | 1612 | GCAGGGUCAGCGACAGCUU | 4510 | AAGCUGUCGCUGACCCUGC |
| siRNA 1613 | 1613 | CAGGGUCAGCGACAGCUUC | 4511 | GAAGCUGUCGCUGACCCUG |
| siRNA 1614 | 1614 | AGGGUCAGCGACAGCUUCU | 4512 | AGAAGCUGUCGCUGACCCU |
| siRNA 1615 | 1615 | GGGUCAGCGACAGCUUCUU | 4513 | AAGAAGCUGUCGCUGACCC |
| siRNA 1616 | 1616 | GGUCAGCGACAGCUUCUUC | 4514 | GAAGAAGCUGUCGCUGACC |
| SIRNA 1617 | 1617 | GUCAGCGACAGCUUCUUCC | 4515 | GGAAGAAGCUGUCGCUGAC |
| siRNA 1618 | 1618 | UCAGCGACAGCUUCUUCCG | 4516 | CGGAAGAAGCUGUCGCUGA |
| siRNA 1619 | 1619 | CAGCGACAGCUUCUUCCGG | 4517 | CCGGAAGAAGCUGUCGCUG |
| siRNA 1620 | 1620 | AGCGACAGCUUCUUCCGGC | 4518 | GCCGGAAGAAGCUGUCGCU |
| siRNA 1621 | 1621 | GCGACAGCUUCUUCCGGCC | 4519 | GGCCGGAAGAAGCUGUCGC |
| siRNA 1622 | 1622 | CGACAGCUUCUUCCGGCCC | 4520 | GGGCCGGAAGAAGCUGUCG |
| siRNA 1623 | 1623 | GACAGCUUCUUCCGGCCCA | 4521 | UGGGCCGGAAGAAGCUGUC |
| siRNA 1624 | 1624 | ACAGCUUCUUCCGGCCCAG | 4522 | CUGGGCCGGAAGAAGCUGU |
| siRNA 1625 | 1625 | CAGCUUCUUCCGGCCCAGC | 4523 | GCUGGGCCGGAAGAAGCUG |
| siRNA 1626 | 1626 | AGCUUCUUCCGGCCCAGCG | 4524 | CGCUGGGCCGGAAGAAGCU |
| siRNA 1627 | 1627 | GCUUCUUCCGGCCCAGCGU | 4525 | ACGCUGGGCCGGAAGAAGC |
| siRNA 1628 | 1628 | CUUCUUCCGGCCCAGCGUC | 4526 | GACGCUGGGCCGGAAGAAG |
| siRNA 1629 | 1629 | UUCUUCCGGCCCAGCGUCA | 4527 | UGACGCUGGGCCGGAAGAA |
| siRNA 1630 | 1630 | UCUUCCGGCCCAGCGUCAU | 4528 | AUGACGCUGGGCCGGAAGA |
| siRNA 1631 | 1631 | CUUCCGGCCCAGCGUCAUG | 4529 | CAUGACGCUGGGCCGGAAG |
| siRNA 1632 | 1632 | UUCCGGCCCAGCGUCAUGG | 4530 | CCAUGACGCUGGGCCGGAA |
| siRNA 1633 | 1633 | UCCGGCCCAGCGUCAUGGA | 4531 | UCCAUGACGCUGGGCCGGA |
| siRNA 1634 | 1634 | CCGGCCCAGCGUCAUGGAG | 4532 | CUCCAUGACGCUGGGCCGG |
| siRNA 1635 | 1635 | CGGCCCAGCGUCAUGGAGC | 4533 | GCUCCAUGACGCUGGGCCG |
| siRNA 1636 | 1636 | GGCCCAGCGUCAUGGAGCC | 4534 | GGCUCCAUGACGCUGGGCC |
| siRNA 1637 | 1637 | GCCCAGCGUCAUGGAGCCC | 4535 | GGGCUCCAUGACGCUGGGC |
| siRNA 1638 | 1638 | CCCAGCGUCAUGGAGCCCA | 4536 | UGGGCUCCAUGACGCUGGG |
| siRNA 1639 | 1639 | CCAGCGUCAUGGAGCCCAU | 4537 | AUGGGCUCCAUGACGCUGG |
| siRNA 1640 | 1640 | CAGCGUCAUGGAGCCCAUC | 4538 | GAUGGGCUCCAUGACGCUG |
| siRNA 1641 | 1641 | AGCGUCAUGGAGCCCAUCC | 4539 | GGAUGGGCUCCAUGACGCU |
| siRNA 1642 | 1642 | GCGUCAUGGAGCCCAUCCU | 4540 | AGGAUGGGCUCCAUGACGC |
| siRNA 1643 | 1643 | CGUCAUGGAGCCCAUCCUG | 4541 | CAGGAUGGGCUCCAUGACG |
| siRNA 1644 | 1644 | GUCAUGGAGCCCAUCCUGG | 4542 | CCAGGAUGGGCUCCAUGAC |
| siRNA 1645 | 1645 | UCAUGGAGCCCAUCCUGGG | 4543 | CCCAGGAUGGGCUCCAUGA |
| siRNA 1646 | 1646 | CAUGGAGCCCAUCCUGGGC | 4544 | GCCCAGGAUGGGCUCCAUG |
| siRNA 1647 | 1647 | AUGGAGCCCAUCCUGGGCC | 4545 | GGCCCAGGAUGGGCUCCAU |
| siRNA 1648 | 1648 | UGGAGCCCAUCCUGGGCCG | 4546 | CGGCCCAGGAUGGGCUCCA |
| siRNA 1649 | 1649 | GGAGCCCAUCCUGGGCCGC | 4547 | GCGGCCCAGGAUGGGCUCC |
| siRNA 1650 | 1650 | GAGCCCAUCCUGGGCCGCA | 4548 | UGCGGCCCAGGAUGGGCUC |
| siRNA 1651 | 1651 | AGCCCAUCCUGGGCCGCAC | 4549 | GUGCGGCCCAGGAUGGGCU |
| siRNA 1652 | 1652 | GCCCAUCCUGGGCCGCACG | 4550 | CGUGCGGCCCAGGAUGGGC |
| siRNA 1653 | 1653 | CCCAUCCUGGGCCGCACGC | 4551 | GCGUGCGGCCCAGGAUGGG |
| siRNA 1654 | 1654 | CCAUCCUGGGCCGCACGCA | 4552 | UGCGUGCGGCCCAGGAUGG |
| siRNA 1655 | 1655 | CAUCCUGGGCCGCACGCAU | 4553 | AUGCGUGCGGCCCAGGAUG |
| siRNA 1656 | 1656 | AUCCUGGGCCGCACGCAUU | 4554 | AAUGCGUGCGGCCCAGGAU |
| siRNA 1657 | 1657 | UCCUGGGCCGCACGCAUUA | 4555 | UAAUGCGUGCGGCCCAGGA |
| siRNA 1658 | 1658 | CCUGGGCCGCACGCAUUAC | 4556 | GUAAUGCGUGCGGCCCAGG |
| siRNA 1659 | 1659 | CUGGGCCGCACGCAUUACA | 4557 | UGUAAUGCGUGCGGCCCAG |
| siRNA 1660 | 1660 | UGGGCCGCACGCAUUACAG | 4558 | CUGUAAUGCGUGCGGCCCA |
| siRNA 1661 | 1661 | GGGCCGCACGCAUUACAGC | 4559 | GCUGUAAUGCGUGCGGCCC |
| siRNA 1662 | 1662 | GGCCGCACGCAUUACAGCC | 4560 | GGCUGUAAUGCGUGCGGCC |
| siRNA 1663 | 1663 | GCCGCACGCAUUACAGCCA | 4561 | UGGCUGUAAUGCGUGCGGC |
| siRNA 1664 | 1664 | CCGCACGCAUUACAGCCAG | 4562 | CUGGCUGUAAUGCGUGCGG |
| siRNA 1665 | 1665 | CGCACGCAUUACAGCCAGC | 4563 | GCUGGCUGUAAUGCGUGCG |
| siRNA 1666 | 1666 | GCACGCAUUACAGCCAGCU | 4564 | AGCUGGCUGUAAUGCGUGC |
| siRNA 1667 | 1667 | CACGCAUUACAGCCAGCUG | 4565 | CAGCUGGCUGUAAUGCGUG |
| siRNA 1668 | 1668 | ACGCAUUACAGCCAGCUGC | 4566 | GCAGCUGGCUGUAAUGCGU |
| siRNA 1669 | 1669 | CGCAUUACAGCCAGCUGCG | 4567 | CGCAGCUGGCUGUAAUGCG |
| siRNA 1670 | 1670 | GCAUUACAGCCAGCUGCGC | 4568 | GCGCAGCUGGCUGUAAUGC |
| siRNA 1671 | 1671 | CAUUACAGCCAGCUGCGCA | 4569 | UGCGCAGCUGGCUGUAAUG |
| siRNA 1672 | 1672 | AUUACAGCCAGCUGCGCAA | 4570 | UUGCGCAGCUGGCUGUAAU |
| siRNA 1673 | 1673 | UUACAGCCAGCUGCGCAAG | 4571 | CUUGCGCAGCUGGCUGUAA |
| siRNA 1674 | 1674 | UACAGCCAGCUGCGCAAGA | 4572 | UCUUGCGCAGCUGGCUGUA |
| siRNA 1675 | 1675 | ACAGCCAGCUGCGCAAGAA | 4573 | UUCUUGCGCAGCUGGCUGU |
| siRNA 1676 | 1676 | CAGCCAGCUGCGCAAGAAG | 4574 | CUUCUUGCGCAGCUGGCUG |
| siRNA 1677 | 1677 | AGCCAGCUGCGCAAGAAGA | 4575 | UCUUCUUGCGCAGCUGGCU |
| siRNA 1678 | 1678 | GCCAGCUGCGCAAGAAGAG | 4576 | CUCUUCUUGCGCAGCUGGC |
| siRNA 1679 | 1679 | CCAGCUGCGCAAGAAGAGC | 4577 | GCUCUUCUUGCGCAGCUGG |
| siRNA 1680 | 1680 | CAGCUGCGCAAGAAGAGCU | 4578 | AGCUCUUCUUGCGCAGCUG |
| siRNA 1681 | 1681 | AGCUGCGCAAGAAGAGCUG | 4579 | CAGCUCUUCUUGCGCAGCU |
| siRNA 1682 | 1682 | GCUGCGCAAGAAGAGCUGA | 4580 | UCAGCUCUUCUUGCGCAGC |
| siRNA 1683 | 1683 | CUGCGCAAGAAGAGCUGAG | 4581 | CUCAGCUCUUCUUGCGCAG |
| siRNA 1684 | 1684 | UGCGCAAGAAGAGCUGAGU | 4582 | ACUCAGCUCUUCUUGCGCA |
| siRNA 1685 | 1685 | GCGCAAGAAGAGCUGAGUC | 4583 | GACUCAGCUCUUCUUGCGC |
| siRNA 1686 | 1686 | CGCAAGAAGAGCUGAGUCG | 4584 | CGACUCAGCUCUUCUUGCG |
| siRNA 1687 | 1687 | GCAAGAAGAGCUGAGUCGC | 4585 | GCGACUCAGCUCUUCUUGC |
| siRNA 1688 | 1688 | CAAGAAGAGCUGAGUCGCC | 4586 | GGCGACUCAGCUCUUCUUG |
| siRNA 1689 | 1689 | AAGAAGAGCUGAGUCGCCG | 4587 | CGGCGACUCAGCUCUUCUU |
| siRNA 1690 | 1690 | AGAAGAGCUGAGUCGCCGC | 4588 | GCGGCGACUCAGCUCUUCU |
| siRNA 1691 | 1691 | GAAGAGCUGAGUCGCCGCA | 4589 | UGCGGCGACUCAGCUCUUC |
| siRNA 1692 | 1692 | AAGAGCUGAGUCGCCGCAC | 4590 | GUGCGGCGACUCAGCUCUU |
| siRNA 1693 | 1693 | AGAGCUGAGUCGCCGCACC | 4591 | GGUGCGGCGACUCAGCUCU |
| siRNA 1694 | 1694 | GAGCUGAGUCGCCGCACCA | 4592 | UGGUGCGGCGACUCAGCUC |
| siRNA 1695 | 1695 | AGCUGAGUCGCCGCACCAG | 4593 | CUGGUGCGGCGACUCAGCU |
| siRNA 1696 | 1696 | GCUGAGUCGCCGCACCAGC | 4594 | GCUGGUGCGGCGACUCAGC |
| siRNA 1697 | 1697 | CUGAGUCGCCGCACCAGCC | 4595 | GGCUGGUGCGGCGACUCAG |
| siRNA 1698 | 1698 | UGAGUCGCCGCACCAGCCG | 4596 | CGGCUGGUGCGGCGACUCA |
| siRNA 1699 | 1699 | GAGUCGCCGCACCAGCCGC | 4597 | GCGGCUGGUGCGGCGACUC |
| siRNA 1700 | 1700 | AGUCGCCGCACCAGCCGCC | 4598 | GGCGGCUGGUGCGGCGACU |
| siRNA 1701 | 1701 | GUCGCCGCACCAGCCGCCG | 4599 | CGGCGGCUGGUGCGGCGAC |
| siRNA 1702 | 1702 | UCGCCGCACCAGCCGCCGC | 4600 | GCGGCGGCUGGUGCGGCGA |
| siRNA 1703 | 1703 | CGCCGCACCAGCCGCCGCG | 4601 | CGCGGCGGCUGGUGCGGCG |
| siRNA 1704 | 1704 | GCCGCACCAGCCGCCGCGC | 4602 | GCGCGGCGGCUGGUGCGGC |
| siRNA 1705 | 1705 | CCGCACCAGCCGCCGCGCC | 4603 | GGCGCGGCGGCUGGUGCGG |
| siRNA 1706 | 1706 | CGCACCAGCCGCCGCGCCC | 4604 | GGGCGCGGCGGCUGGUGCG |
| siRNA 1707 | 1707 | GCACCAGCCGCCGCGCCCC | 4605 | GGGGCGCGGCGGCUGGUGC |
| siRNA 1708 | 1708 | CACCAGCCGCCGCGCCCCG | 4606 | CGGGGCGCGGCGGCUGGUG |
| SIRNA 1709 | 1709 | ACCAGCCGCCGCGCCCCGG | 4607 | CCGGGGCGCGGCGGCUGGU |
| siRNA 1710 | 1710 | CCAGCCGCCGCGCCCCGGG | 4608 | CCCGGGGCGCGGCGGCUGG |
| siRNA 1711 | 1711 | CAGCCGCCGCGCCCCGGGC | 4609 | GCCCGGGGCGCGGCGGCUG |
| siRNA 1712 | 1712 | AGCCGCCGCGCCCCGGGCC | 4610 | GGCCCGGGGCGCGGCGGCU |
| siRNA 1713 | 1713 | GCCGCCGCGCCCCGGGCCG | 4611 | CGGCCCGGGGCGCGGCGGC |
| siRNA 1714 | 1714 | CCGCCGCGCCCCGGGCCGG | 4612 | CCGGCCCGGGGCGCGGCGG |
| siRNA 1715 | 1715 | CGCCGCGCCCCGGGCCGGC | 4613 | GCCGGCCCGGGGCGCGGCG |
| siRNA 1716 | 1716 | GCCGCGCCCCGGGCCGGCG | 4614 | CGCCGGCCCGGGGCGCGGC |
| siRNA 1717 | 1717 | CCGCGCCCCGGGCCGGCGG | 4615 | CCGCCGGCCCGGGGCGCGG |
| siRNA 1718 | 1718 | CGCGCCCCGGGCCGGCGGG | 4616 | CCCGCCGGCCCGGGGCGCG |
| siRNA 1719 | 1719 | GCGCCCCGGGCCGGCGGGU | 4617 | ACCCGCCGGCCCGGGGCGC |
| siRNA 1720 | 1720 | CGCCCCGGGCCGGCGGGUU | 4618 | AACCCGCCGGCCCGGGGCG |
| siRNA 1721 | 1721 | GCCCCGGGCCGGCGGGUUU | 4619 | AAACCCGCCGGCCCGGGGC |
| siRNA 1722 | 1722 | CCCCGGGCCGGCGGGUUUC | 4620 | GAAACCCGCCGGCCCGGGG |
| siRNA 1723 | 1723 | CCCGGGCCGGCGGGUUUCU | 4621 | AGAAACCCGCCGGCCCGGG |
| siRNA 1724 | 1724 | CCGGGCCGGCGGGUUUCUC | 4622 | GAGAAACCCGCCGGCCCGG |
| SIRNA 1725 | 1725 | CGGGCCGGCGGGUUUCUCU | 4623 | AGAGAAACCCGCCGGCCCG |
| siRNA 1726 | 1726 | GGGCCGGCGGGUUUCUCUA | 4624 | UAGAGAAACCCGCCGGCCC |
| siRNA 1727 | 1727 | GGCCGGCGGGUUUCUCUAA | 4625 | UUAGAGAAACCCGCCGGCC |
| siRNA 1728 | 1728 | GCCGGCGGGUUUCUCUAAC | 4626 | GUUAGAGAAACCCGCCGGC |
| siRNA 1729 | 1729 | CCGGCGGGUUUCUCUAACA | 4627 | UGUUAGAGAAACCCGCCGG |
| siRNA 1730 | 1730 | CGGCGGGUUUCUCUAACAA | 4628 | UUGUUAGAGAAACCCGCCG |
| siRNA 1731 | 1731 | GGCGGGUUUCUCUAACAAA | 4629 | UUUGUUAGAGAAACCCGCC |
| siRNA 1732 | 1732 | GCGGGUUUCUCUAACAAAU | 4630 | AUUUGUUAGAGAAACCCGC |
| siRNA 1733 | 1733 | CGGGUUUCUCUAACAAAUA | 4631 | UAUUUGUUAGAGAAACCCG |
| siRNA 1734 | 1734 | GGGUUUCUCUAACAAAUAA | 4632 | UUAUUUGUUAGAGAAACCC |
| siRNA 1735 | 1735 | GGUUUCUCUAACAAAUAAA | 4633 | UUUAUUUGUUAGAGAAACC |
| siRNA 1736 | 1736 | GUUUCUCUAACAAAUAAAC | 4634 | GUUUAUUUGUUAGAGAAAC |
| siRNA 1737 | 1737 | UUUCUCUAACAAAUAAACA | 4635 | UGUUUAUUUGUUAGAGAAA |
| siRNA 1738 | 1738 | UUCUCUAACAAAUAAACAG | 4636 | CUGUUUAUUUGUUAGAGAA |
| siRNA 1739 | 1739 | UCUCUAACAAAUAAACAGA | 4637 | UCUGUUUAUUUGUUAGAGA |
| siRNA 1740 | 1740 | CUCUAACAAAUAAACAGAA | 4638 | UUCUGUUUAUUUGUUAGAG |
| siRNA 1741 | 1741 | UCUAACAAAUAAACAGAAC | 4639 | GUUCUGUUUAUUUGUUAGA |
| siRNA 1742 | 1742 | CUAACAAAUAAACAGAACC | 4640 | GGUUCUGUUUAUUUGUUAG |
| siRNA 1743 | 1743 | UAACAAAUAAACAGAACCC | 4641 | GGGUUCUGUUUAUUUGUUA |
| siRNA 1744 | 1744 | AACAAAUAAACAGAACCCG | 4642 | CGGGUUCUGUUUAUUUGUU |
| siRNA 1745 | 1745 | ACAAAUAAACAGAACCCGC | 4643 | GCGGGUUCUGUUUAUUUGU |
| siRNA 1746 | 1746 | CAAAUAAACAGAACCCGCA | 4644 | UGCGGGUUCUGUUUAUUUG |
| siRNA 1747 | 1747 | AAAUAAACAGAACCCGCAC | 4645 | GUGCGGGUUCUGUUUAUUU |
| siRNA 1748 | 1748 | AAUAAACAGAACCCGCACU | 4646 | AGUGCGGGUUCUGUUUAUU |
| siRNA 1749 | 1749 | AUAAACAGAACCCGCACUG | 4647 | CAGUGCGGGUUCUGUUUAU |
| siRNA 1750 | 1750 | UAAACAGAACCCGCACUGC | 4648 | GCAGUGCGGGUUCUGUUUA |
| siRNA 1751 | 1751 | AAACAGAACCCGCACUGCC | 4649 | GGCAGUGCGGGUUCUGUUU |
| siRNA 1752 | 1752 | AACAGAACCCGCACUGCCC | 4650 | GGGCAGUGCGGGUUCUGUU |
| siRNA 1753 | 1753 | ACAGAACCCGCACUGCCCA | 4651 | UGGGCAGUGCGGGUUCUGU |
| siRNA 1754 | 1754 | CAGAACCCGCACUGCCCAG | 4652 | CUGGGCAGUGCGGGUUCUG |
| siRNA 1755 | 1755 | AGAACCCGCACUGCCCAGG | 4653 | CCUGGGCAGUGCGGGUUCU |
| siRNA 1756 | 1756 | GAACCCGCACUGCCCAGGC | 4654 | GCCUGGGCAGUGCGGGUUC |
| SIRNA 1757 | 1757 | AACCCGCACUGCCCAGGCG | 4655 | CGCCUGGGCAGUGCGGGUU |
| siRNA 1758 | 1758 | ACCCGCACUGCCCAGGCGA | 4656 | UCGCCUGGGCAGUGCGGGU |
| siRNA 1759 | 1759 | CCCGCACUGCCCAGGCGAG | 4657 | CUCGCCUGGGCAGUGCGGG |
| siRNA 1760 | 1760 | CCGCACUGCCCAGGCGAGC | 4658 | GCUCGCCUGGGCAGUGCGG |
| siRNA 1761 | 1761 | CGCACUGCCCAGGCGAGCG | 4659 | CGCUCGCCUGGGCAGUGCG |
| siRNA 1762 | 1762 | GCACUGCCCAGGCGAGCGU | 4660 | ACGCUCGCCUGGGCAGUGC |
| siRNA 1763 | 1763 | CACUGCCCAGGCGAGCGUU | 4661 | AACGCUCGCCUGGGCAGUG |
| siRNA 1764 | 1764 | ACUGCCCAGGCGAGCGUUG | 4662 | CAACGCUCGCCUGGGCAGU |
| siRNA 1765 | 1765 | CUGCCCAGGCGAGCGUUGC | 4663 | GCAACGCUCGCCUGGGCAG |
| siRNA 1766 | 1766 | UGCCCAGGCGAGCGUUGCC | 4664 | GGCAACGCUCGCCUGGGCA |
| siRNA 1767 | 1767 | GCCCAGGCGAGCGUUGCCA | 4665 | UGGCAACGCUCGCCUGGGC |
| siRNA 1768 | 1768 | CCCAGGCGAGCGUUGCCAC | 4666 | GUGGCAACGCUCGCCUGGG |
| siRNA 1769 | 1769 | CCAGGCGAGCGUUGCCACU | 4667 | AGUGGCAACGCUCGCCUGG |
| siRNA 1770 | 1770 | CAGGCGAGCGUUGCCACUU | 4668 | AAGUGGCAACGCUCGCCUG |
| siRNA 1771 | 1771 | AGGCGAGCGUUGCCACUUU | 4669 | AAAGUGGCAACGCUCGCCU |
| siRNA 1772 | 1772 | GGCGAGCGUUGCCACUUUC | 4670 | GAAAGUGGCAACGCUCGCC |
| siRNA 1773 | 1773 | GCGAGCGUUGCCACUUUCA | 4671 | UGAAAGUGGCAACGCUCGC |
| siRNA 1774 | 1774 | CGAGCGUUGCCACUUUCAA | 4672 | UUGAAAGUGGCAACGCUCG |
| siRNA 1775 | 1775 | GAGCGUUGCCACUUUCAAA | 4673 | UUUGAAAGUGGCAACGCUC |
| siRNA 1776 | 1776 | AGCGUUGCCACUUUCAAAG | 4674 | CUUUGAAAGUGGCAACGCU |
| siRNA 1777 | 1777 | GCGUUGCCACUUUCAAAGU | 4675 | ACUUUGAAAGUGGCAACGC |
| siRNA 1778 | 1778 | CGUUGCCACUUUCAAAGUG | 4676 | CACUUUGAAAGUGGCAACG |
| siRNA 1779 | 1779 | GUUGCCACUUUCAAAGUGG | 4677 | CCACUUUGAAAGUGGCAAC |
| siRNA 1780 | 1780 | UUGCCACUUUCAAAGUGGU | 4678 | ACCACUUUGAAAGUGGCAA |
| siRNA 1781 | 1781 | UGCCACUUUCAAAGUGGUC | 4679 | GACCACUUUGAAAGUGGCA |
| siRNA 1782 | 1782 | GCCACUUUCAAAGUGGUCC | 4680 | GGACCACUUUGAAAGUGGC |
| siRNA 1783 | 1783 | CCACUUUCAAAGUGGUCCC | 4681 | GGGACCACUUUGAAAGUGG |
| siRNA 1784 | 1784 | CACUUUCAAAGUGGUCCCC | 4682 | GGGGACCACUUUGAAAGUG |
| siRNA 1785 | 1785 | ACUUUCAAAGUGGUCCCCU | 4683 | AGGGGACCACUUUGAAAGU |
| siRNA 1786 | 1786 | CUUUCAAAGUGGUCCCCUG | 4684 | CAGGGGACCACUUUGAAAG |
| siRNA 1787 | 1787 | UUUCAAAGUGGUCCCCUGG | 4685 | CCAGGGGACCACUUUGAAA |
| siRNA 1788 | 1788 | UUCAAAGUGGUCCCCUGGG | 4686 | CCCAGGGGACCACUUUGAA |
| siRNA 1789 | 1789 | UCAAAGUGGUCCCCUGGGG | 4687 | CCCCAGGGGACCACUUUGA |
| siRNA 1790 | 1790 | CAAAGUGGUCCCCUGGGGA | 4688 | UCCCCAGGGGACCACUUUG |
| siRNA 1791 | 1791 | AAAGUGGUCCCCUGGGGAG | 4689 | CUCCCCAGGGGACCACUUU |
| siRNA 1792 | 1792 | AAGUGGUCCCCUGGGGAGC | 4690 | GCUCCCCAGGGGACCACUU |
| siRNA 1793 | 1793 | AGUGGUCCCCUGGGGAGCU | 4691 | AGCUCCCCAGGGGACCACU |
| siRNA 1794 | 1794 | GUGGUCCCCUGGGGAGCUC | 4692 | GAGCUCCCCAGGGGACCAC |
| siRNA 1795 | 1795 | UGGUCCCCUGGGGAGCUCA | 4693 | UGAGCUCCCCAGGGGACCA |
| siRNA 1796 | 1796 | GGUCCCCUGGGGAGCUCAG | 4694 | CUGAGCUCCCCAGGGGACC |
| siRNA 1797 | 1797 | GUCCCCUGGGGAGCUCAGC | 4695 | GCUGAGCUCCCCAGGGGAC |
| siRNA 1798 | 1798 | UCCCCUGGGGAGCUCAGCC | 4696 | GGCUGAGCUCCCCAGGGGA |
| siRNA 1799 | 1799 | CCCCUGGGGAGCUCAGCCU | 4697 | AGGCUGAGCUCCCCAGGGG |
| siRNA 1800 | 1800 | CCCUGGGGAGCUCAGCCUC | 4698 | GAGGCUGAGCUCCCCAGGG |
| siRNA 1801 | 1801 | CCUGGGGAGCUCAGCCUCA | 4699 | UGAGGCUGAGCUCCCCAGG |
| siRNA 1802 | 1802 | CUGGGGAGCUCAGCCUCAU | 4700 | AUGAGGCUGAGCUCCCCAG |
| siRNA 1803 | 1803 | UGGGGAGCUCAGCCUCAUC | 4701 | GAUGAGGCUGAGCUCCCCA |
| siRNA 1804 | 1804 | GGGGAGCUCAGCCUCAUCC | 4702 | GGAUGAGGCUGAGCUCCCC |
| siRNA 1805 | 1805 | GGGAGCUCAGCCUCAUCCU | 4703 | AGGAUGAGGCUGAGCUCCC |
| siRNA 1806 | 1806 | GGAGCUCAGCCUCAUCCUG | 4704 | CAGGAUGAGGCUGAGCUCC |
| siRNA 1807 | 1807 | GAGCUCAGCCUCAUCCUGA | 4705 | UCAGGAUGAGGCUGAGCUC |
| siRNA 1808 | 1808 | AGCUCAGCCUCAUCCUGAU | 4706 | AUCAGGAUGAGGCUGAGCU |
| siRNA 1809 | 1809 | GCUCAGCCUCAUCCUGAUG | 4707 | CAUCAGGAUGAGGCUGAGC |
| SIRNA 1810 | 1810 | CUCAGCCUCAUCCUGAUGA | 4708 | UCAUCAGGAUGAGGCUGAG |
| siRNA 1811 | 1811 | UCAGCCUCAUCCUGAUGAU | 4709 | AUCAUCAGGAUGAGGCUGA |
| siRNA 1812 | 1812 | CAGCCUCAUCCUGAUGAUG | 4710 | CAUCAUCAGGAUGAGGCUG |
| siRNA 1813 | 1813 | AGCCUCAUCCUGAUGAUGC | 4711 | GCAUCAUCAGGAUGAGGCU |
| siRNA 1814 | 1814 | GCCUCAUCCUGAUGAUGCU | 4712 | AGCAUCAUCAGGAUGAGGC |
| siRNA 1815 | 1815 | CCUCAUCCUGAUGAUGCUG | 4713 | CAGCAUCAUCAGGAUGAGG |
| siRNA 1816 | 1816 | CUCAUCCUGAUGAUGCUGC | 4714 | GCAGCAUCAUCAGGAUGAG |
| siRNA 1817 | 1817 | UCAUCCUGAUGAUGCUGCC | 4715 | GGCAGCAUCAUCAGGAUGA |
| siRNA 1818 | 1818 | CAUCCUGAUGAUGCUGCCA | 4716 | UGGCAGCAUCAUCAGGAUG |
| siRNA 1819 | 1819 | AUCCUGAUGAUGCUGCCAA | 4717 | UUGGCAGCAUCAUCAGGAU |
| siRNA 1820 | 1820 | UCCUGAUGAUGCUGCCAAG | 4718 | CUUGGCAGCAUCAUCAGGA |
| siRNA 1821 | 1821 | CCUGAUGAUGCUGCCAAGG | 4719 | CCUUGGCAGCAUCAUCAGG |
| siRNA 1822 | 1822 | CUGAUGAUGCUGCCAAGGC | 4720 | GCCUUGGCAGCAUCAUCAG |
| siRNA 1823 | 1823 | UGAUGAUGCUGCCAAGGCG | 4721 | CGCCUUGGCAGCAUCAUCA |
| siRNA 1824 | 1824 | GAUGAUGCUGCCAAGGCGC | 4722 | GCGCCUUGGCAGCAUCAUC |
| siRNA 1825 | 1825 | AUGAUGCUGCCAAGGCGCA | 4723 | UGCGCCUUGGCAGCAUCAU |
| siRNA 1826 | 1826 | UGAUGCUGCCAAGGCGCAC | 4724 | GUGCGCCUUGGCAGCAUCA |
| siRNA 1827 | 1827 | GAUGCUGCCAAGGCGCACU | 4725 | AGUGCGCCUUGGCAGCAUC |
| siRNA 1828 | 1828 | AUGCUGCCAAGGCGCACUU | 4726 | AAGUGCGCCUUGGCAGCAU |
| siRNA 1829 | 1829 | UGCUGCCAAGGCGCACUUU | 4727 | AAAGUGCGCCUUGGCAGCA |
| siRNA 1830 | 1830 | GCUGCCAAGGCGCACUUUU | 4728 | AAAAGUGCGCCUUGGCAGC |
| siRNA 1831 | 1831 | CUGCCAAGGCGCACUUUUU | 4729 | AAAAAGUGCGCCUUGGCAG |
| siRNA 1832 | 1832 | UGCCAAGGCGCACUUUUUA | 4730 | UAAAAAGUGCGCCUUGGCA |
| siRNA 1833 | 1833 | GCCAAGGCGCACUUUUUAU | 4731 | AUAAAAAGUGCGCCUUGGC |
| siRNA 1834 | 1834 | CCAAGGCGCACUUUUUAUU | 4732 | AAUAAAAAGUGCGCCUUGG |
| siRNA 1835 | 1835 | CAAGGCGCACUUUUUAUUU | 4733 | AAAUAAAAAGUGCGCCUUG |
| siRNA 1836 | 1836 | AAGGCGCACUUUUUAUUUU | 4734 | AAAAUAAAAAGUGCGCCUU |
| siRNA 1837 | 1837 | AGGCGCACUUUUUUUUUU | 4735 | AAAAAUAAAAAGUGCGCCU |
| siRNA 1838 | 1838 | GGCGCACUUUUUAUUUUUA | 4736 | UAAAAAUAAAAAGUGCGCC |
| siRNA 1839 | 1839 | GCGCACUUUUUAUUUUUAU | 4737 | AUAAAAAUAAAAAGUGCGC |
| siRNA 1840 | 1840 | CGCACUUUUUAUUUUUAUU | 4738 | AAUAAAAAUAAAAAGUGCG |
| siRNA 1841 | 1841 | GCACUUUUUAUUUUUAUUU | 4739 | AAAUAAAAAUAAAAAGUGC |
| siRNA 1842 | 1842 | CACUUUUUAUUUUUAUUUU | 4740 | AAAAUAAAAAUAAAAAGUG |
| siRNA 1843 | 1843 | ACUUUUUAUUUUUAUUUUA | 4741 | UAAAAUAAAAAUAAAAAGU |
| siRNA 1844 | 1844 | CUUUUUAUUUUUAUUUUAU | 4742 | AUAAAAUAAAAAUAAAAAG |
| siRNA 1845 | 1845 | UUUUUAUUUUUAUUUUAUU | 4743 | AAUAAAAUAAAAAUAAAAA |
| SIRNA 1846 | 1846 | UUUUAUUUUUAUUUUAUUU | 4744 | AAAUAAAAUAAAAAUAAAA |
| siRNA 1847 | 1847 | UUUAUUUUUAUUUUAUUUU | 4745 | AAAAUAAAAAUAAAAUAAA |
| siRNA 1848 | 1848 | UUAUUUUUAUUUUAUUUUU | 4746 | AAAAAUAAAAUAAAAAUAA |
| siRNA 1849 | 1849 | UAUUUUUAUUUUAUUUUUA | 4747 | UAAAAAUAAAAUAAAAAUA |
| siRNA 1850 | 1850 | AUUUUUAUUUUAUUUUUAU | 4748 | AUAAAAAUAAAAUAAAAAU |
| siRNA 1851 | 1851 | UUUUUAUUUUAUUUUUAUU | 4749 | AAUAAAAAUAAAAUAAAAA |
| siRNA 1852 | 1852 | UUUUAUUUUAUUUUUAUUU | 4750 | AAAUAAAAAUAAAAUAAAA |
| siRNA 1853 | 1853 | UUUAUUUUAUUUUUAUUUU | 4751 | AAAAUAAAAAUAAAAUAAA |
| siRNA 1854 | 1854 | UUAUUUUAUUUUUAUUUUU | 4752 | AAAAAUAAAAAUAAAAUAA |
| siRNA 1855 | 1855 | UAUUUUAUUUUUAUUUUUU | 4753 | AAAAAAUAAAAAUAAAAUA |
| siRNA 1856 | 1856 | AUUUUAUUUUUAUUUUUUU | 4754 | AAAAAAAUAAAAAUAAAAU |
| siRNA 1857 | 1857 | UUUUAUUUUUAUUUUUUUU | 4755 | AAAAAAAAUAAAAAUAAAA |
| siRNA 1858 | 1858 | UUUAUUUUUAUUUUUUUUU | 4756 | AAAAAAAAAUAAAAAUAAA |
| siRNA 1859 | 1859 | UUAUUUUUAUUUUUUUUUU | 4757 | AAAAAAAAAAUAAAAAUAA |
| siRNA 1860 | 1860 | UAUUUUUAUUUUUUUUUUA | 4758 | UAAAAAAAAAAUAAAAAUA |
| siRNA 1861 | 1861 | AUUUUUAUUUUUUUUUUAG | 4759 | CUAAAAAAAAAAUAAAAAU |
| siRNA 1862 | 1862 | UUUUUAUUUUUUUUUUAGC | 4760 | GCUAAAAAAAAAAUAAAAA |
| siRNA 1863 | 1863 | UUUUAUUUUUUUUUUAGCA | 4761 | UGCUAAAAAAAAAAUAAAA |
| siRNA 1864 | 1864 | UUUAUUUUUUUUUUAGCAU | 4762 | AUGCUAAAAAAAAAAUAAA |
| siRNA 1865 | 1865 | UUAUUUUUUUUUUAGCAUC | 4763 | GAUGCUAAAAAAAAAAUAA |
| siRNA 1866 | 1866 | UAUUUUUUUUUUAGCAUCC | 4764 | GGAUGCUAAAAAAAAAAUA |
| siRNA 1867 | 1867 | AUUUUUUUUUUAGCAUCCU | 4765 | AGGAUGCUAAAAAAAAAAU |
| siRNA 1868 | 1868 | UUUUUUUUUUAGCAUCCUU | 4766 | AAGGAUGCUAAAAAAAAAA |
| siRNA 1869 | 1869 | UUUUUUUUUAGCAUCCUUU | 4767 | AAAGGAUGCUAAAAAAAAA |
| siRNA 1870 | 1870 | UUUUUUUUAGCAUCCUUUU | 4768 | AAAAGGAUGCUAAAAAAAA |
| siRNA 1871 | 1871 | UUUUUUUAGCAUCCUUUUG | 4769 | CAAAAGGAUGCUAAAAAAA |
| siRNA 1872 | 1872 | UUUUUUAGCAUCCUUUUGG | 4770 | CCAAAAGGAUGCUAAAAAA |
| siRNA 1873 | 1873 | UUUUUAGCAUCCUUUUGGG | 4771 | CCCAAAAGGAUGCUAAAAA |
| siRNA 1874 | 1874 | UUUUAGCAUCCUUUUGGGG | 4772 | CCCCAAAAGGAUGCUAAAA |
| siRNA 1875 | 1875 | UUUAGCAUCCUUUUGGGGC | 4773 | GCCCCAAAAGGAUGCUAAA |
| siRNA 1876 | 1876 | UUAGCAUCCUUUUGGGGCU | 4774 | AGCCCCAAAAGGAUGCUAA |
| siRNA 1877 | 1877 | UAGCAUCCUUUUGGGGCUU | 4775 | AAGCCCCAAAAGGAUGCUA |
| siRNA 1878 | 1878 | AGCAUCCUUUUGGGGCUUC | 4776 | GAAGCCCCAAAAGGAUGCU |
| siRNA 1879 | 1879 | GCAUCCUUUUGGGGCUUCA | 4777 | UGAAGCCCCAAAAGGAUGC |
| siRNA 1880 | 1880 | CAUCCUUUUGGGGCUUCAC | 4778 | GUGAAGCCCCAAAAGGAUG |
| siRNA 1881 | 1881 | AUCCUUUUGGGGCUUCACU | 4779 | AGUGAAGCCCCAAAAGGAU |
| siRNA 1882 | 1882 | UCCUUUUGGGGCUUCACUC | 4780 | GAGUGAAGCCCCAAAAGGA |
| siRNA 1883 | 1883 | CCUUUUGGGGCUUCACUCU | 4781 | AGAGUGAAGCCCCAAAAGG |
| siRNA 1884 | 1884 | CUUUUGGGGCUUCACUCUC | 4782 | GAGAGUGAAGCCCCAAAAG |
| siRNA 1885 | 1885 | UUUUGGGGCUUCACUCUCA | 4783 | UGAGAGUGAAGCCCCAAAA |
| siRNA 1886 | 1886 | UUUGGGGCUUCACUCUCAG | 4784 | CUGAGAGUGAAGCCCCAAA |
| siRNA 1887 | 1887 | UUGGGGCUUCACUCUCAGA | 4785 | UCUGAGAGUGAAGCCCCAA |
| siRNA 1888 | 1888 | UGGGGCUUCACUCUCAGAG | 4786 | CUCUGAGAGUGAAGCCCCA |
| siRNA 1889 | 1889 | GGGGCUUCACUCUCAGAGC | 4787 | GCUCUGAGAGUGAAGCCCC |
| siRNA 1890 | 1890 | GGGCUUCACUCUCAGAGCC | 4788 | GGCUCUGAGAGUGAAGCCC |
| siRNA 1891 | 1891 | GGCUUCACUCUCAGAGCCA | 4789 | UGGCUCUGAGAGUGAAGCC |
| siRNA 1892 | 1892 | GCUUCACUCUCAGAGCCAG | 4790 | CUGGCUCUGAGAGUGAAGC |
| siRNA 1893 | 1893 | CUUCACUCUCAGAGCCAGU | 4791 | ACUGGCUCUGAGAGUGAAG |
| siRNA 1894 | 1894 | UUCACUCUCAGAGCCAGUU | 4792 | AACUGGCUCUGAGAGUGAA |
| siRNA 1895 | 1895 | UCACUCUCAGAGCCAGUUU | 4793 | AAACUGGCUCUGAGAGUGA |
| siRNA 1896 | 1896 | CACUCUCAGAGCCAGUUUU | 4794 | AAAACUGGCUCUGAGAGUG |
| siRNA 1897 | 1897 | ACUCUCAGAGCCAGUUUUU | 4795 | AAAAACUGGCUCUGAGAGU |
| siRNA 1898 | 1898 | CUCUCAGAGCCAGUUUUUA | 4796 | UAAAAACUGGCUCUGAGAG |
| siRNA 1899 | 1899 | UCUCAGAGCCAGUUUUUAA | 4797 | UUAAAAACUGGCUCUGAGA |
| siRNA 1900 | 1900 | CUCAGAGCCAGUUUUUAAG | 4798 | CUUAAAAACUGGCUCUGAG |
| siRNA 1901 | 1901 | UCAGAGCCAGUUUUUAAGG | 4799 | CCUUAAAAACUGGCUCUGA |
| siRNA 1902 | 1902 | CAGAGCCAGUUUUUAAGGG | 4800 | CCCUUAAAAACUGGCUCUG |
| siRNA 1903 | 1903 | AGAGCCAGUUUUUAAGGGA | 4801 | UCCCUUAAAAACUGGCUCU |
| siRNA 1904 | 1904 | GAGCCAGUUUUUAAGGGAC | 4802 | GUCCCUUAAAAACUGGCUC |
| siRNA 1905 | 1905 | AGCCAGUUUUUAAGGGACA | 4803 | UGUCCCUUAAAAACUGGCU |
| siRNA 1906 | 1906 | GCCAGUUUUUAAGGGACAC | 4804 | GUGUCCCUUAAAAACUGGC |
| siRNA 1907 | 1907 | CCAGUUUUUAAGGGACACC | 4805 | GGUGUCCCUUAAAAACUGG |
| siRNA 1908 | 1908 | CAGUUUUUAAGGGACACCA | 4806 | UGGUGUCCCUUAAAAACUG |
| siRNA 1909 | 1909 | AGUUUUUAAGGGACACCAG | 4807 | CUGGUGUCCCUUAAAAACU |
| siRNA 1910 | 1910 | GUUUUUAAGGGACACCAGA | 4808 | UCUGGUGUCCCUUAAAAAC |
| siRNA 1911 | 1911 | UUUUUAAGGGACACCAGAG | 4809 | CUCUGGUGUCCCUUAAAAA |
| siRNA 1912 | 1912 | UUUUAAGGGACACCAGAGC | 4810 | GCUCUGGUGUCCCUUAAAA |
| siRNA 1913 | 1913 | UUUAAGGGACACCAGAGCC | 4811 | GGCUCUGGUGUCCCUUAAA |
| siRNA 1914 | 1914 | UUAAGGGACACCAGAGCCG | 4812 | CGGCUCUGGUGUCCCUUAA |
| siRNA 1915 | 1915 | UAAGGGACACCAGAGCCGC | 4813 | GCGGCUCUGGUGUCCCUUA |
| siRNA 1916 | 1916 | AAGGGACACCAGAGCCGCA | 4814 | UGCGGCUCUGGUGUCCCUU |
| siRNA 1917 | 1917 | AGGGACACCAGAGCCGCAG | 4815 | CUGCGGCUCUGGUGUCCCU |
| siRNA 1918 | 1918 | GGGACACCAGAGCCGCAGC | 4816 | GCUGCGGCUCUGGUGUCCC |
| siRNA 1919 | 1919 | GGACACCAGAGCCGCAGCC | 4817 | GGCUGCGGCUCUGGUGUCC |
| siRNA 1920 | 1920 | GACACCAGAGCCGCAGCCU | 4818 | AGGCUGCGGCUCUGGUGUC |
| siRNA 1921 | 1921 | ACACCAGAGCCGCAGCCUG | 4819 | CAGGCUGCGGCUCUGGUGU |
| siRNA 1922 | 1922 | CACCAGAGCCGCAGCCUGC | 4820 | GCAGGCUGCGGCUCUGGUG |
| siRNA 1923 | 1923 | ACCAGAGCCGCAGCCUGCU | 4821 | AGCAGGCUGCGGCUCUGGU |
| siRNA 1924 | 1924 | CCAGAGCCGCAGCCUGCUC | 4822 | GAGCAGGCUGCGGCUCUGG |
| siRNA 1925 | 1925 | CAGAGCCGCAGCCUGCUCU | 4823 | AGAGCAGGCUGCGGCUCUG |
| siRNA 1926 | 1926 | AGAGCCGCAGCCUGCUCUG | 4824 | CAGAGCAGGCUGCGGCUCU |
| siRNA 1927 | 1927 | GAGCCGCAGCCUGCUCUGA | 4825 | UCAGAGCAGGCUGCGGCUC |
| siRNA 1928 | 1928 | AGCCGCAGCCUGCUCUGAU | 4826 | AUCAGAGCAGGCUGCGGCU |
| siRNA 1929 | 1929 | GCCGCAGCCUGCUCUGAUU | 4827 | AAUCAGAGCAGGCUGCGGC |
| siRNA 1930 | 1930 | CCGCAGCCUGCUCUGAUUC | 4828 | GAAUCAGAGCAGGCUGCGG |
| siRNA 1931 | 1931 | CGCAGCCUGCUCUGAUUCU | 4829 | AGAAUCAGAGCAGGCUGCG |
| siRNA 1932 | 1932 | GCAGCCUGCUCUGAUUCUA | 4830 | UAGAAUCAGAGCAGGCUGC |
| SIRNA 1933 | 1933 | CAGCCUGCUCUGAUUCUAU | 4831 | AUAGAAUCAGAGCAGGCUG |
| siRNA 1934 | 1934 | AGCCUGCUCUGAUUCUAUG | 4832 | CAUAGAAUCAGAGCAGGCU |
| siRNA 1935 | 1935 | GCCUGCUCUGAUUCUAUGG | 4833 | CCAUAGAAUCAGAGCAGGC |
| siRNA 1936 | 1936 | CCUGCUCUGAUUCUAUGGC | 4834 | GCCAUAGAAUCAGAGCAGG |
| siRNA 1937 | 1937 | CUGCUCUGAUUCUAUGGCU | 4835 | AGCCAUAGAAUCAGAGCAG |
| siRNA 1938 | 1938 | UGCUCUGAUUCUAUGGCUU | 4836 | AAGCCAUAGAAUCAGAGCA |
| siRNA 1939 | 1939 | GCUCUGAUUCUAUGGCUUG | 4837 | CAAGCCAUAGAAUCAGAGC |
| siRNA 1940 | 1940 | CUCUGAUUCUAUGGCUUGG | 4838 | CCAAGCCAUAGAAUCAGAG |
| siRNA 1941 | 1941 | UCUGAUUCUAUGGCUUGGU | 4839 | ACCAAGCCAUAGAAUCAGA |
| siRNA 1942 | 1942 | CUGAUUCUAUGGCUUGGUU | 4840 | AACCAAGCCAUAGAAUCAG |
| siRNA 1943 | 1943 | UGAUUCUAUGGCUUGGUUG | 4841 | CAACCAAGCCAUAGAAUCA |
| siRNA 1944 | 1944 | GAUUCUAUGGCUUGGUUGU | 4842 | ACAACCAAGCCAUAGAAUC |
| siRNA 1945 | 1945 | AUUCUAUGGCUUGGUUGUU | 4843 | AACAACCAAGCCAUAGAAU |
| siRNA 1946 | 1946 | UUCUAUGGCUUGGUUGUUA | 4844 | UAACAACCAAGCCAUAGAA |
| siRNA 1947 | 1947 | UCUAUGGCUUGGUUGUUAC | 4845 | GUAACAACCAAGCCAUAGA |
| siRNA 1948 | 1948 | CUAUGGCUUGGUUGUUACU | 4846 | AGUAACAACCAAGCCAUAG |
| siRNA 1949 | 1949 | UAUGGCUUGGUUGUUACUA | 4847 | UAGUAACAACCAAGCCAUA |
| siRNA 1950 | 1950 | AUGGCUUGGUUGUUACUAU | 4848 | AUAGUAACAACCAAGCCAU |
| siRNA 1951 | 1951 | UGGCUUGGUUGUUACUAUA | 4849 | UAUAGUAACAACCAAGCCA |
| siRNA 1952 | 1952 | GGCUUGGUUGUUACUAUAA | 4850 | UUAUAGUAACAACCAAGCC |
| siRNA 1953 | 1953 | GCUUGGUUGUUACUAUAAG | 4851 | CUUAUAGUAACAACCAAGC |
| siRNA 1954 | 1954 | CUUGGUUGUUACUAUAAGA | 4852 | UCUUAUAGUAACAACCAAG |
| siRNA 1955 | 1955 | UUGGUUGUUACUAUAAGAG | 4853 | CUCUUAUAGUAACAACCAA |
| siRNA 1956 | 1956 | UGGUUGUUACUAUAAGAGU | 4854 | ACUCUUAUAGUAACAACCA |
| siRNA 1957 | 1957 | GGUUGUUACUAUAAGAGUA | 4855 | UACUCUUAUAGUAACAACC |
| siRNA 1958 | 1958 | GUUGUUACUAUAAGAGUAA | 4856 | UUACUCUUAUAGUAACAAC |
| siRNA 1959 | 1959 | UUGUUACUAUAAGAGUAAU | 4857 | AUUACUCUUAUAGUAACAA |
| siRNA 1960 | 1960 | UGUUACUAUAAGAGUAAUU | 4858 | AAUUACUCUUAUAGUAACA |
| siRNA 1961 | 1961 | GUUACUAUAAGAGUAAUUG | 4859 | CAAUUACUCUUAUAGUAAC |
| siRNA 1962 | 1962 | UUACUAUAAGAGUAAUUGC | 4860 | GCAAUUACUCUUAUAGUAA |
| siRNA 1963 | 1963 | UACUAUAAGAGUAAUUGCC | 4861 | GGCAAUUACUCUUAUAGUA |
| siRNA 1964 | 1964 | ACUAUAAGAGUAAUUGCCU | 4862 | AGGCAAUUACUCUUAUAGU |
| siRNA 1965 | 1965 | CUAUAAGAGUAAUUGCCUA | 4863 | UAGGCAAUUACUCUUAUAG |
| siRNA 1966 | 1966 | UAUAAGAGUAAUUGCCUAA | 4864 | UUAGGCAAUUACUCUUAUA |
| siRNA 1967 | 1967 | AUAAGAGUAAUUGCCUAAC | 4865 | GUUAGGCAAUUACUCUUAU |
| siRNA 1968 | 1968 | UAAGAGUAAUUGCCUAACU | 4866 | AGUUAGGCAAUUACUCUUA |
| siRNA 1969 | 1969 | AAGAGUAAUUGCCUAACUU | 4867 | AAGUUAGGCAAUUACUCUU |
| siRNA 1970 | 1970 | AGAGUAAUUGCCUAACUUG | 4868 | CAAGUUAGGCAAUUACUCU |
| siRNA 1971 | 1971 | GAGUAAUUGCCUAACUUGA | 4869 | UCAAGUUAGGCAAUUACUC |
| siRNA 1972 | 1972 | AGUAAUUGCCUAACUUGAU | 4870 | AUCAAGUUAGGCAAUUACU |
| siRNA 1973 | 1973 | GUAAUUGCCUAACUUGAUU | 4871 | AAUCAAGUUAGGCAAUUAC |
| siRNA 1974 | 1974 | UAAUUGCCUAACUUGAUUU | 4872 | AAAUCAAGUUAGGCAAUUA |
| siRNA 1975 | 1975 | AAUUGCCUAACUUGAUUUU | 4873 | AAAAUCAAGUUAGGCAAUU |
| siRNA 1976 | 1976 | AUUGCCUAACUUGAUUUUU | 4874 | AAAAAUCAAGUUAGGCAAU |
| siRNA 1977 | 1977 | UUGCCUAACUUGAUUUUUC | 4875 | GAAAAAUCAAGUUAGGCAA |
| siRNA 1978 | 1978 | UGCCUAACUUGAUUUUUCA | 4876 | UGAAAAAUCAAGUUAGGCA |
| siRNA 1979 | 1979 | GCCUAACUUGAUUUUUCAU | 4877 | AUGAAAAAUCAAGUUAGGC |
| siRNA 1980 | 1980 | CCUAACUUGAUUUUUCAUC | 4878 | GAUGAAAAAUCAAGUUAGG |
| siRNA 1981 | 1981 | CUAACUUGAUUUUUCAUCU | 4879 | AGAUGAAAAAUCAAGUUAG |
| siRNA 1982 | 1982 | UAACUUGAUUUUUCAUCUC | 4880 | GAGAUGAAAAAUCAAGUUA |
| siRNA 1983 | 1983 | AACUUGAUUUUUCAUCUCU | 4881 | AGAGAUGAAAAAUCAAGUU |
| siRNA 1984 | 1984 | ACUUGAUUUUUCAUCUCUU | 4882 | AAGAGAUGAAAAAUCAAGU |
| siRNA 1985 | 1985 | CUUGAUUUUUCAUCUCUUU | 4883 | AAAGAGAUGAAAAAUCAAG |
| siRNA 1986 | 1986 | UUGAUUUUUCAUCUCUUUA | 4884 | UAAAGAGAUGAAAAAUCAA |
| siRNA 1987 | 1987 | UGAUUUUUCAUCUCUUUAA | 4885 | UUAAAGAGAUGAAAAAUCA |
| siRNA 1988 | 1988 | GAUUUUUCAUCUCUUUAAC | 4886 | GUUAAAGAGAUGAAAAAUC |
| siRNA 1989 | 1989 | AUUUUUCAUCUCUUUAACC | 4887 | GGUUAAAGAGAUGAAAAAU |
| siRNA 1990 | 1990 | UUUUUCAUCUCUUUAACCA | 4888 | UGGUUAAAGAGAUGAAAAA |
| siRNA 1991 | 1991 | UUUUCAUCUCUUUAACCAA | 4889 | UUGGUUAAAGAGAUGAAAA |
| siRNA 1992 | 1992 | UUUCAUCUCUUUAACCAAA | 4890 | UUUGGUUAAAGAGAUGAAA |
| siRNA 1993 | 1993 | UUCAUCUCUUUAACCAAAC | 4891 | GUUUGGUUAAAGAGAUGAA |
| siRNA 1994 | 1994 | UCAUCUCUUUAACCAAACU | 4892 | AGUUUGGUUAAAGAGAUGA |
| siRNA 1995 | 1995 | CAUCUCUUUAACCAAACUU | 4893 | AAGUUUGGUUAAAGAGAUG |
| siRNA 1996 | 1996 | AUCUCUUUAACCAAACUUG | 4894 | CAAGUUUGGUUAAAGAGAU |
| siRNA 1997 | 1997 | UCUCUUUAACCAAACUUGU | 4895 | ACAAGUUUGGUUAAAGAGA |
| siRNA 1998 | 1998 | CUCUUUAACCAAACUUGUG | 4896 | CACAAGUUUGGUUAAAGAG |
| siRNA 1999 | 1999 | UCUUUAACCAAACUUGUGG | 4897 | CCACAAGUUUGGUUAAAGA |
| siRNA 2000 | 2000 | CUUUAACCAAACUUGUGGC | 4898 | GCCACAAGUUUGGUUAAAG |
| siRNA 2001 | 2001 | UUUAACCAAACUUGUGGCC | 4899 | GGCCACAAGUUUGGUUAAA |
| siRNA 2002 | 2002 | UUAACCAAACUUGUGGCCA | 4900 | UGGCCACAAGUUUGGUUAA |
| siRNA 2003 | 2003 | UAACCAAACUUGUGGCCAA | 4901 | UUGGCCACAAGUUUGGUUA |
| siRNA 2004 | 2004 | AACCAAACUUGUGGCCAAA | 4902 | UUUGGCCACAAGUUUGGUU |
| siRNA 2005 | 2005 | ACCAAACUUGUGGCCAAAA | 4903 | UUUUGGCCACAAGUUUGGU |
| siRNA 2006 | 2006 | CCAAACUUGUGGCCAAAAG | 4904 | CUUUUGGCCACAAGUUUGG |
| siRNA 2007 | 2007 | CAAACUUGUGGCCAAAAGA | 4905 | UCUUUUGGCCACAAGUUUG |
| siRNA 2008 | 2008 | AAACUUGUGGCCAAAAGAU | 4906 | AUCUUUUGGCCACAAGUUU |
| siRNA 2009 | 2009 | AACUUGUGGCCAAAAGAUA | 4907 | UAUCUUUUGGCCACAAGUU |
| siRNA 2010 | 2010 | ACUUGUGGCCAAAAGAUAU | 4908 | AUAUCUUUUGGCCACAAGU |
| siRNA 2011 | 2011 | CUUGUGGCCAAAAGAUAUU | 4909 | AAUAUCUUUUGGCCACAAG |
| siRNA 2012 | 2012 | UUGUGGCCAAAAGAUAUUU | 4910 | AAAUAUCUUUUGGCCACAA |
| siRNA 2013 | 2013 | UGUGGCCAAAAGAUAUUUG | 4911 | CAAAUAUCUUUUGGCCACA |
| siRNA 2014 | 2014 | GUGGCCAAAAGAUAUUUGA | 4912 | UCAAAUAUCUUUUGGCCAC |
| siRNA 2015 | 2015 | UGGCCAAAAGAUAUUUGAC | 4913 | GUCAAAUAUCUUUUGGCCA |
| siRNA 2016 | 2016 | GGCCAAAAGAUAUUUGACC | 4914 | GGUCAAAUAUCUUUUGGCC |
| siRNA 2017 | 2017 | GCCAAAAGAUAUUUGACCG | 4915 | CGGUCAAAUAUCUUUUGGC |
| siRNA 2018 | 2018 | CCAAAAGAUAUUUGACCGU | 4916 | ACGGUCAAAUAUCUUUUGG |
| siRNA 2019 | 2019 | CAAAAGAUAUUUGACCGUU | 4917 | AACGGUCAAAUAUCUUUUG |
| siRNA 2020 | 2020 | AAAAGAUAUUUGACCGUUU | 4918 | AAACGGUCAAAUAUCUUUU |
| siRNA 2021 | 2021 | AAAGAUAUUUGACCGUUUC | 4919 | GAAACGGUCAAAUAUCUUU |
| siRNA 2022 | 2022 | AAGAUAUUUGACCGUUUCC | 4920 | GGAAACGGUCAAAUAUCUU |
| siRNA 2023 | 2023 | AGAUAUUUGACCGUUUCCA | 4921 | UGGAAACGGUCAAAUAUCU |
| siRNA 2024 | 2024 | GAUAUUUGACCGUUUCCAA | 4922 | UUGGAAACGGUCAAAUAUC |
| siRNA 2025 | 2025 | AUAUUUGACCGUUUCCAAA | 4923 | UUUGGAAACGGUCAAAUAU |
| siRNA 2026 | 2026 | UAUUUGACCGUUUCCAAAA | 4924 | UUUUGGAAACGGUCAAAUA |
| siRNA 2027 | 2027 | AUUUGACCGUUUCCAAAAU | 4925 | AUUUUGGAAACGGUCAAAU |
| siRNA 2028 | 2028 | UUUGACCGUUUCCAAAAUU | 4926 | AAUUUUGGAAACGGUCAAA |
| siRNA 2029 | 2029 | UUGACCGUUUCCAAAAUUC | 4927 | GAAUUUUGGAAACGGUCAA |
| siRNA 2030 | 2030 | UGACCGUUUCCAAAAUUCA | 4928 | UGAAUUUUGGAAACGGUCA |
| siRNA 2031 | 2031 | GACCGUUUCCAAAAUUCAG | 4929 | CUGAAUUUUGGAAACGGUC |
| siRNA 2032 | 2032 | ACCGUUUCCAAAAUUCAGA | 4930 | UCUGAAUUUUGGAAACGGU |
| siRNA 2033 | 2033 | CCGUUUCCAAAAUUCAGAU | 4931 | AUCUGAAUUUUGGAAACGG |
| siRNA 2034 | 2034 | CGUUUCCAAAAUUCAGAUU | 4932 | AAUCUGAAUUUUGGAAACG |
| siRNA 2035 | 2035 | GUUUCCAAAAUUCAGAUUC | 4933 | GAAUCUGAAUUUUGGAAAC |
| siRNA 2036 | 2036 | UUUCCAAAAUUCAGAUUCU | 4934 | AGAAUCUGAAUUUUGGAAA |
| siRNA 2037 | 2037 | UUCCAAAAUUCAGAUUCUG | 4935 | CAGAAUCUGAAUUUUGGAA |
| siRNA 2038 | 2038 | UCCAAAAUUCAGAUUCUGC | 4936 | GCAGAAUCUGAAUUUUGGA |
| siRNA 2039 | 2039 | CCAAAAUUCAGAUUCUGCC | 4937 | GGCAGAAUCUGAAUUUUGG |
| siRNA 2040 | 2040 | CAAAAUUCAGAUUCUGCCU | 4938 | AGGCAGAAUCUGAAUUUUG |
| siRNA 2041 | 2041 | AAAAUUCAGAUUCUGCCUC | 4939 | GAGGCAGAAUCUGAAUUUU |
| siRNA 2042 | 2042 | AAAUUCAGAUUCUGCCUCU | 4940 | AGAGGCAGAAUCUGAAUUU |
| siRNA 2043 | 2043 | AAUUCAGAUUCUGCCUCUG | 4941 | CAGAGGCAGAAUCUGAAUU |
| siRNA 2044 | 2044 | AUUCAGAUUCUGCCUCUGC | 4942 | GCAGAGGCAGAAUCUGAAU |
| siRNA 2045 | 2045 | UUCAGAUUCUGCCUCUGCG | 4943 | CGCAGAGGCAGAAUCUGAA |
| siRNA 2046 | 2046 | UCAGAUUCUGCCUCUGCGG | 4944 | CCGCAGAGGCAGAAUCUGA |
| siRNA 2047 | 2047 | CAGAUUCUGCCUCUGCGGA | 4945 | UCCGCAGAGGCAGAAUCUG |
| siRNA 2048 | 2048 | AGAUUCUGCCUCUGCGGAU | 4946 | AUCCGCAGAGGCAGAAUCU |
| siRNA 2049 | 2049 | GAUUCUGCCUCUGCGGAUA | 4947 | UAUCCGCAGAGGCAGAAUC |
| siRNA 2050 | 2050 | AUUCUGCCUCUGCGGAUAA | 4948 | UUAUCCGCAGAGGCAGAAU |
| siRNA 2051 | 2051 | UUCUGCCUCUGCGGAUAAA | 4949 | UUUAUCCGCAGAGGCAGAA |
| siRNA 2052 | 2052 | UCUGCCUCUGCGGAUAAAU | 4950 | AUUUAUCCGCAGAGGCAGA |
| siRNA 2053 | 2053 | CUGCCUCUGCGGAUAAAUA | 4951 | UAUUUAUCCGCAGAGGCAG |
| siRNA 2054 | 2054 | UGCCUCUGCGGAUAAAUAU | 4952 | AUAUUUAUCCGCAGAGGCA |
| siRNA 2055 | 2055 | GCCUCUGCGGAUAAAUAUU | 4953 | AAUAUUUAUCCGCAGAGGC |
| siRNA 2056 | 2056 | CCUCUGCGGAUAAAUAUUU | 4954 | AAAUAUUUAUCCGCAGAGG |
| siRNA 2057 | 2057 | CUCUGCGGAUAAAUAUUUG | 4955 | CAAAUAUUUAUCCGCAGAG |
| siRNA 2058 | 2058 | UCUGCGGAUAAAUAUUUGC | 4956 | GCAAAUAUUUAUCCGCAGA |
| siRNA 2059 | 2059 | CUGCGGAUAAAUAUUUGCC | 4957 | GGCAAAUAUUUAUCCGCAG |
| siRNA 2060 | 2060 | UGCGGAUAAAUAUUUGCCA | 4958 | UGGCAAAUAUUUAUCCGCA |
| siRNA 2061 | 2061 | GCGGAUAAAUAUUUGCCAC | 4959 | GUGGCAAAUAUUUAUCCGC |
| siRNA 2062 | 2062 | CGGAUAAAUAUUUGCCACG | 4960 | CGUGGCAAAUAUUUAUCCG |
| siRNA 2063 | 2063 | GGAUAAAUAUUUGCCACGA | 4961 | UCGUGGCAAAUAUUUAUCC |
| siRNA 2064 | 2064 | GAUAAAUAUUUGCCACGAA | 4962 | UUCGUGGCAAAUAUUUAUC |
| siRNA 2065 | 2065 | AUAAAUAUUUGCCACGAAU | 4963 | AUUCGUGGCAAAUAUUUAU |
| siRNA 2066 | 2066 | UAAAUAUUUGCCACGAAUG | 4964 | CAUUCGUGGCAAAUAUUUA |
| siRNA 2067 | 2067 | AAAUAUUUGCCACGAAUGA | 4965 | UCAUUCGUGGCAAAUAUUU |
| siRNA 2068 | 2068 | AAUAUUUGCCACGAAUGAG | 4966 | CUCAUUCGUGGCAAAUAUU |
| siRNA 2069 | 2069 | AUAUUUGCCACGAAUGAGU | 4967 | ACUCAUUCGUGGCAAAUAU |
| siRNA 2070 | 2070 | UAUUUGCCACGAAUGAGUA | 4968 | UACUCAUUCGUGGCAAAUA |
| siRNA 2071 | 2071 | AUUUGCCACGAAUGAGUAA | 4969 | UUACUCAUUCGUGGCAAAU |
| siRNA 2072 | 2072 | UUUGCCACGAAUGAGUAAC | 4970 | GUUACUCAUUCGUGGCAAA |
| siRNA 2073 | 2073 | UUGCCACGAAUGAGUAACU | 4971 | AGUUACUCAUUCGUGGCAA |
| siRNA 2074 | 2074 | UGCCACGAAUGAGUAACUC | 4972 | GAGUUACUCAUUCGUGGCA |
| siRNA 2075 | 2075 | GCCACGAAUGAGUAACUCC | 4973 | GGAGUUACUCAUUCGUGGC |
| siRNA 2076 | 2076 | CCACGAAUGAGUAACUCCU | 4974 | AGGAGUUACUCAUUCGUGG |
| siRNA 2077 | 2077 | CACGAAUGAGUAACUCCUG | 4975 | CAGGAGUUACUCAUUCGUG |
| siRNA 2078 | 2078 | ACGAAUGAGUAACUCCUGU | 4976 | ACAGGAGUUACUCAUUCGU |
| siRNA 2079 | 2079 | CGAAUGAGUAACUCCUGUC | 4977 | GACAGGAGUUACUCAUUCG |
| siRNA 2080 | 2080 | GAAUGAGUAACUCCUGUCA | 4978 | UGACAGGAGUUACUCAUUC |
| siRNA 2081 | 2081 | AAUGAGUAACUCCUGUCAC | 4979 | GUGACAGGAGUUACUCAUU |
| siRNA 2082 | 2082 | AUGAGUAACUCCUGUCACC | 4980 | GGUGACAGGAGUUACUCAU |
| siRNA 2083 | 2083 | UGAGUAACUCCUGUCACCA | 4981 | UGGUGACAGGAGUUACUCA |
| siRNA 2084 | 2084 | GAGUAACUCCUGUCACCAC | 4982 | GUGGUGACAGGAGUUACUC |
| siRNA 2085 | 2085 | AGUAACUCCUGUCACCACU | 4983 | AGUGGUGACAGGAGUUACU |
| siRNA 2086 | 2086 | GUAACUCCUGUCACCACUC | 4984 | GAGUGGUGACAGGAGUUAC |
| siRNA 2087 | 2087 | UAACUCCUGUCACCACUCU | 4985 | AGAGUGGUGACAGGAGUUA |
| siRNA 2088 | 2088 | AACUCCUGUCACCACUCUG | 4986 | CAGAGUGGUGACAGGAGUU |
| siRNA 2089 | 2089 | ACUCCUGUCACCACUCUGA | 4987 | UCAGAGUGGUGACAGGAGU |
| siRNA 2090 | 2090 | CUCCUGUCACCACUCUGAA | 4988 | UUCAGAGUGGUGACAGGAG |
| siRNA 2091 | 2091 | UCCUGUCACCACUCUGAAG | 4989 | CUUCAGAGUGGUGACAGGA |
| siRNA 2092 | 2092 | CCUGUCACCACUCUGAAGG | 4990 | CCUUCAGAGUGGUGACAGG |
| siRNA 2093 | 2093 | CUGUCACCACUCUGAAGGU | 4991 | ACCUUCAGAGUGGUGACAG |
| siRNA 2094 | 2094 | UGUCACCACUCUGAAGGUC | 4992 | GACCUUCAGAGUGGUGACA |
| siRNA 2095 | 2095 | GUCACCACUCUGAAGGUCC | 4993 | GGACCUUCAGAGUGGUGAC |
| siRNA 2096 | 2096 | UCACCACUCUGAAGGUCCA | 4994 | UGGACCUUCAGAGUGGUGA |
| siRNA 2097 | 2097 | CACCACUCUGAAGGUCCAG | 4995 | CUGGACCUUCAGAGUGGUG |
| siRNA 2098 | 2098 | ACCACUCUGAAGGUCCAGA | 4996 | UCUGGACCUUCAGAGUGGU |
| siRNA 2099 | 2099 | CCACUCUGAAGGUCCAGAC | 4997 | GUCUGGACCUUCAGAGUGG |
| siRNA 2100 | 2100 | CACUCUGAAGGUCCAGACA | 4998 | UGUCUGGACCUUCAGAGUG |
| siRNA 2101 | 2101 | ACUCUGAAGGUCCAGACAG | 4999 | CUGUCUGGACCUUCAGAGU |
| siRNA 2102 | 2102 | CUCUGAAGGUCCAGACAGA | 5000 | UCUGUCUGGACCUUCAGAG |
| siRNA 2103 | 2103 | UCUGAAGGUCCAGACAGAA | 5001 | UUCUGUCUGGACCUUCAGA |
| siRNA 2104 | 2104 | CUGAAGGUCCAGACAGAAG | 5002 | CUUCUGUCUGGACCUUCAG |
| siRNA 2105 | 2105 | UGAAGGUCCAGACAGAAGG | 5003 | CCUUCUGUCUGGACCUUCA |
| siRNA 2106 | 2106 | GAAGGUCCAGACAGAAGGU | 5004 | ACCUUCUGUCUGGACCUUC |
| siRNA 2107 | 2107 | AAGGUCCAGACAGAAGGUU | 5005 | AACCUUCUGUCUGGACCUU |
| siRNA 2108 | 2108 | AGGUCCAGACAGAAGGUUU | 5006 | AAACCUUCUGUCUGGACCU |
| SiRNA 2109 | 2109 | GGUCCAGACAGAAGGUUUU | 5007 | AAAACCUUCUGUCUGGACC |
| siRNA 2110 | 2110 | GUCCAGACAGAAGGUUUUG | 5008 | CAAAACCUUCUGUCUGGAC |
| siRNA 2111 | 2111 | UCCAGACAGAAGGUUUUGA | 5009 | UCAAAACCUUCUGUCUGGA |
| siRNA 2112 | 2112 | CCAGACAGAAGGUUUUGAC | 5010 | GUCAAAACCUUCUGUCUGG |
| siRNA 2113 | 2113 | CAGACAGAAGGUUUUGACA | 5011 | UGUCAAAACCUUCUGUCUG |
| siRNA 2114 | 2114 | AGACAGAAGGUUUUGACAC | 5012 | GUGUCAAAACCUUCUGUCU |
| siRNA 2115 | 2115 | GACAGAAGGUUUUGACACA | 5013 | UGUGUCAAAACCUUCUGUC |
| siRNA 2116 | 2116 | ACAGAAGGUUUUGACACAU | 5014 | AUGUGUCAAAACCUUCUGU |
| siRNA 2117 | 2117 | CAGAAGGUUUUGACACAUU | 5015 | AAUGUGUCAAAACCUUCUG |
| siRNA 2118 | 2118 | AGAAGGUUUUGACACAUUC | 5016 | GAAUGUGUCAAAACCUUCU |
| siRNA 2119 | 2119 | GAAGGUUUUGACACAUUCU | 5017 | AGAAUGUGUCAAAACCUUC |
| siRNA 2120 | 2120 | AAGGUUUUGACACAUUCUU | 5018 | AAGAAUGUGUCAAAACCUU |
| siRNA 2121 | 2121 | AGGUUUUGACACAUUCUUA | 5019 | UAAGAAUGUGUCAAAACCU |
| siRNA 2122 | 2122 | GGUUUUGACACAUUCUUAG | 5020 | CUAAGAAUGUGUCAAAACC |
| siRNA 2123 | 2123 | GUUUUGACACAUUCUUAGC | 5021 | GCUAAGAAUGUGUCAAAAC |
| siRNA 2124 | 2124 | UUUUGACACAUUCUUAGCA | 5022 | UGCUAAGAAUGUGUCAAAA |
| siRNA 2125 | 2125 | UUUGACACAUUCUUAGCAC | 5023 | GUGCUAAGAAUGUGUCAAA |
| siRNA 2126 | 2126 | UUGACACAUUCUUAGCACU | 5024 | AGUGCUAAGAAUGUGUCAA |
| siRNA 2127 | 2127 | UGACACAUUCUUAGCACUG | 5025 | CAGUGCUAAGAAUGUGUCA |
| siRNA 2128 | 2128 | GACACAUUCUUAGCACUGA | 5026 | UCAGUGCUAAGAAUGUGUC |
| siRNA 2129 | 2129 | ACACAUUCUUAGCACUGAA | 5027 | UUCAGUGCUAAGAAUGUGU |
| siRNA 2130 | 2130 | CACAUUCUUAGCACUGAAC | 5028 | GUUCAGUGCUAAGAAUGUG |
| siRNA 2131 | 2131 | ACAUUCUUAGCACUGAACU | 5029 | AGUUCAGUGCUAAGAAUGU |
| siRNA 2132 | 2132 | CAUUCUUAGCACUGAACUC | 5030 | GAGUUCAGUGCUAAGAAUG |
| siRNA 2133 | 2133 | AUUCUUAGCACUGAACUCC | 5031 | GGAGUUCAGUGCUAAGAAU |
| siRNA 2134 | 2134 | UUCUUAGCACUGAACUCCU | 5032 | AGGAGUUCAGUGCUAAGAA |
| siRNA 2135 | 2135 | UCUUAGCACUGAACUCCUC | 5033 | GAGGAGUUCAGUGCUAAGA |
| siRNA 2136 | 2136 | CUUAGCACUGAACUCCUCU | 5034 | AGAGGAGUUCAGUGCUAAG |
| siRNA 2137 | 2137 | UUAGCACUGAACUCCUCUG | 5035 | CAGAGGAGUUCAGUGCUAA |
| siRNA 2138 | 2138 | UAGCACUGAACUCCUCUGU | 5036 | ACAGAGGAGUUCAGUGCUA |
| siRNA 2139 | 2139 | AGCACUGAACUCCUCUGUG | 5037 | CACAGAGGAGUUCAGUGCU |
| siRNA 2140 | 2140 | GCACUGAACUCCUCUGUGA | 5038 | UCACAGAGGAGUUCAGUGC |
| siRNA 2141 | 2141 | CACUGAACUCCUCUGUGAU | 5039 | AUCACAGAGGAGUUCAGUG |
| siRNA 2142 | 2142 | ACUGAACUCCUCUGUGAUC | 5040 | GAUCACAGAGGAGUUCAGU |
| siRNA 2143 | 2143 | CUGAACUCCUCUGUGAUCU | 5041 | AGAUCACAGAGGAGUUCAG |
| siRNA 2144 | 2144 | UGAACUCCUCUGUGAUCUA | 5042 | UAGAUCACAGAGGAGUUCA |
| siRNA 2145 | 2145 | GAACUCCUCUGUGAUCUAG | 5043 | CUAGAUCACAGAGGAGUUC |
| siRNA 2146 | 2146 | AACUCCUCUGUGAUCUAGG | 5044 | CCUAGAUCACAGAGGAGUU |
| siRNA 2147 | 2147 | ACUCCUCUGUGAUCUAGGA | 5045 | UCCUAGAUCACAGAGGAGU |
| siRNA 2148 | 2148 | CUCCUCUGUGAUCUAGGAU | 5046 | AUCCUAGAUCACAGAGGAG |
| siRNA 2149 | 2149 | UCCUCUGUGAUCUAGGAUG | 5047 | CAUCCUAGAUCACAGAGGA |
| siRNA 2150 | 2150 | CCUCUGUGAUCUAGGAUGA | 5048 | UCAUCCUAGAUCACAGAGG |
| siRNA 2151 | 2151 | CUCUGUGAUCUAGGAUGAU | 5049 | AUCAUCCUAGAUCACAGAG |
| siRNA 2152 | 2152 | UCUGUGAUCUAGGAUGAUC | 5050 | GAUCAUCCUAGAUCACAGA |
| siRNA 2153 | 2153 | CUGUGAUCUAGGAUGAUCU | 5051 | AGAUCAUCCUAGAUCACAG |
| siRNA 2154 | 2154 | UGUGAUCUAGGAUGAUCUG | 5052 | CAGAUCAUCCUAGAUCACA |
| siRNA 2155 | 2155 | GUGAUCUAGGAUGAUCUGU | 5053 | ACAGAUCAUCCUAGAUCAC |
| siRNA 2156 | 2156 | UGAUCUAGGAUGAUCUGUU | 5054 | AACAGAUCAUCCUAGAUCA |
| siRNA 2157 | 2157 | GAUCUAGGAUGAUCUGUUC | 5055 | GAACAGAUCAUCCUAGAUC |
| siRNA 2158 | 2158 | AUCUAGGAUGAUCUGUUCC | 5056 | GGAACAGAUCAUCCUAGAU |
| siRNA 2159 | 2159 | UCUAGGAUGAUCUGUUCCC | 5057 | GGGAACAGAUCAUCCUAGA |
| siRNA 2160 | 2160 | CUAGGAUGAUCUGUUCCCC | 5058 | GGGGAACAGAUCAUCCUAG |
| siRNA 2161 | 2161 | UAGGAUGAUCUGUUCCCCC | 5059 | GGGGGAACAGAUCAUCCUA |
| siRNA 2162 | 2162 | AGGAUGAUCUGUUCCCCCU | 5060 | AGGGGGAACAGAUCAUCCU |
| siRNA 2163 | 2163 | GGAUGAUCUGUUCCCCCUC | 5061 | GAGGGGGAACAGAUCAUCC |
| siRNA 2164 | 2164 | GAUGAUCUGUUCCCCCUCU | 5062 | AGAGGGGGAACAGAUCAUC |
| siRNA 2165 | 2165 | AUGAUCUGUUCCCCCUCUG | 5063 | CAGAGGGGGAACAGAUCAU |
| siRNA 2166 | 2166 | UGAUCUGUUCCCCCUCUGA | 5064 | UCAGAGGGGGAACAGAUCA |
| siRNA 2167 | 2167 | GAUCUGUUCCCCCUCUGAU | 5065 | AUCAGAGGGGGAACAGAUC |
| siRNA 2168 | 2168 | AUCUGUUCCCCCUCUGAUG | 5066 | CAUCAGAGGGGGAACAGAU |
| siRNA 2169 | 2169 | UCUGUUCCCCCUCUGAUGA | 5067 | UCAUCAGAGGGGGAACAGA |
| siRNA 2170 | 2170 | CUGUUCCCCCUCUGAUGAA | 5068 | UUCAUCAGAGGGGGAACAG |
| siRNA 2171 | 2171 | UGUUCCCCCUCUGAUGAAC | 5069 | GUUCAUCAGAGGGGGAACA |
| siRNA 2172 | 2172 | GUUCCCCCUCUGAUGAACA | 5070 | UGUUCAUCAGAGGGGGAAC |
| siRNA 2173 | 2173 | UUCCCCCUCUGAUGAACAU | 5071 | AUGUUCAUCAGAGGGGGAA |
| siRNA 2174 | 2174 | UCCCCCUCUGAUGAACAUC | 5072 | GAUGUUCAUCAGAGGGGGA |
| siRNA 2175 | 2175 | CCCCCUCUGAUGAACAUCC | 5073 | GGAUGUUCAUCAGAGGGGG |
| siRNA 2176 | 2176 | CCCCUCUGAUGAACAUCCU | 5074 | AGGAUGUUCAUCAGAGGGG |
| siRNA 2177 | 2177 | CCCUCUGAUGAACAUCCUC | 5075 | GAGGAUGUUCAUCAGAGGG |
| siRNA 2178 | 2178 | CCUCUGAUGAACAUCCUCU | 5076 | AGAGGAUGUUCAUCAGAGG |
| siRNA 2179 | 2179 | CUCUGAUGAACAUCCUCUG | 5077 | CAGAGGAUGUUCAUCAGAG |
| siRNA 2180 | 2180 | UCUGAUGAACAUCCUCUGA | 5078 | UCAGAGGAUGUUCAUCAGA |
| siRNA 2181 | 2181 | CUGAUGAACAUCCUCUGAU | 5079 | AUCAGAGGAUGUUCAUCAG |
| siRNA 2182 | 2182 | UGAUGAACAUCCUCUGAUG | 5080 | CAUCAGAGGAUGUUCAUCA |
| siRNA 2183 | 2183 | GAUGAACAUCCUCUGAUGA | 5081 | UCAUCAGAGGAUGUUCAUC |
| siRNA 2184 | 2184 | AUGAACAUCCUCUGAUGAU | 5082 | AUCAUCAGAGGAUGUUCAU |
| siRNA 2185 | 2185 | UGAACAUCCUCUGAUGAUC | 5083 | GAUCAUCAGAGGAUGUUCA |
| siRNA 2186 | 2186 | GAACAUCCUCUGAUGAUCU | 5084 | AGAUCAUCAGAGGAUGUUC |
| siRNA 2187 | 2187 | AACAUCCUCUGAUGAUCUA | 5085 | UAGAUCAUCAGAGGAUGUU |
| siRNA 2188 | 2188 | ACAUCCUCUGAUGAUCUAG | 5086 | CUAGAUCAUCAGAGGAUGU |
| siRNA 2189 | 2189 | CAUCCUCUGAUGAUCUAGG | 5087 | CCUAGAUCAUCAGAGGAUG |
| siRNA 2190 | 2190 | AUCCUCUGAUGAUCUAGGC | 5088 | GCCUAGAUCAUCAGAGGAU |
| siRNA 2191 | 2191 | UCCUCUGAUGAUCUAGGCU | 5089 | AGCCUAGAUCAUCAGAGGA |
| siRNA 2192 | 2192 | CCUCUGAUGAUCUAGGCUC | 5090 | GAGCCUAGAUCAUCAGAGG |
| siRNA 2193 | 2193 | CUCUGAUGAUCUAGGCUCC | 5091 | GGAGCCUAGAUCAUCAGAG |
| siRNA 2194 | 2194 | UCUGAUGAUCUAGGCUCCC | 5092 | GGGAGCCUAGAUCAUCAGA |
| siRNA 2195 | 2195 | CUGAUGAUCUAGGCUCCCA | 5093 | UGGGAGCCUAGAUCAUCAG |
| siRNA 2196 | 2196 | UGAUGAUCUAGGCUCCCAG | 5094 | CUGGGAGCCUAGAUCAUCA |
| siRNA 2197 | 2197 | GAUGAUCUAGGCUCCCAGC | 5095 | GCUGGGAGCCUAGAUCAUC |
| siRNA 2198 | 2198 | AUGAUCUAGGCUCCCAGCA | 5096 | UGCUGGGAGCCUAGAUCAU |
| siRNA 2199 | 2199 | UGAUCUAGGCUCCCAGCAG | 5097 | CUGCUGGGAGCCUAGAUCA |
| SIRNA 2200 | 2200 | GAUCUAGGCUCCCAGCAGG | 5098 | CCUGCUGGGAGCCUAGAUC |
| siRNA 2201 | 2201 | AUCUAGGCUCCCAGCAGGC | 5099 | GCCUGCUGGGAGCCUAGAU |
| siRNA 2202 | 2202 | UCUAGGCUCCCAGCAGGCU | 5100 | AGCCUGCUGGGAGCCUAGA |
| siRNA 2203 | 2203 | CUAGGCUCCCAGCAGGCUA | 5101 | UAGCCUGCUGGGAGCCUAG |
| siRNA 2204 | 2204 | UAGGCUCCCAGCAGGCUAC | 5102 | GUAGCCUGCUGGGAGCCUA |
| siRNA 2205 | 2205 | AGGCUCCCAGCAGGCUACU | 5103 | AGUAGCCUGCUGGGAGCCU |
| siRNA 2206 | 2206 | GGCUCCCAGCAGGCUACUU | 5104 | AAGUAGCCUGCUGGGAGCC |
| siRNA 2207 | 2207 | GCUCCCAGCAGGCUACUUU | 5105 | AAAGUAGCCUGCUGGGAGC |
| siRNA 2208 | 2208 | CUCCCAGCAGGCUACUUUG | 5106 | CAAAGUAGCCUGCUGGGAG |
| siRNA 2209 | 2209 | UCCCAGCAGGCUACUUUGA | 5107 | UCAAAGUAGCCUGCUGGGA |
| siRNA 2210 | 2210 | CCCAGCAGGCUACUUUGAA | 5108 | UUCAAAGUAGCCUGCUGGG |
| siRNA 2211 | 2211 | CCAGCAGGCUACUUUGAAG | 5109 | CUUCAAAGUAGCCUGCUGG |
| siRNA 2212 | 2212 | CAGCAGGCUACUUUGAAGG | 5110 | CCUUCAAAGUAGCCUGCUG |
| siRNA 2213 | 2213 | AGCAGGCUACUUUGAAGGG | 5111 | CCCUUCAAAGUAGCCUGCU |
| siRNA 2214 | 2214 | GCAGGCUACUUUGAAGGGA | 5112 | UCCCUUCAAAGUAGCCUGC |
| siRNA 2215 | 2215 | CAGGCUACUUUGAAGGGAA | 5113 | UUCCCUUCAAAGUAGCCUG |
| siRNA 2216 | 2216 | AGGCUACUUUGAAGGGAAC | 5114 | GUUCCCUUCAAAGUAGCCU |
| siRNA 2217 | 2217 | GGCUACUUUGAAGGGAACA | 5115 | UGUUCCCUUCAAAGUAGCC |
| siRNA 2218 | 2218 | GCUACUUUGAAGGGAACAA | 5116 | UUGUUCCCUUCAAAGUAGC |
| siRNA 2219 | 2219 | CUACUUUGAAGGGAACAAU | 5117 | AUUGUUCCCUUCAAAGUAG |
| siRNA 2220 | 2220 | UACUUUGAAGGGAACAAUC | 5118 | GAUUGUUCCCUUCAAAGUA |
| siRNA 2221 | 2221 | ACUUUGAAGGGAACAAUCA | 5119 | UGAUUGUUCCCUUCAAAGU |
| siRNA 2222 | 2222 | CUUUGAAGGGAACAAUCAG | 5120 | CUGAUUGUUCCCUUCAAAG |
| siRNA 2223 | 2223 | UUUGAAGGGAACAAUCAGA | 5121 | UCUGAUUGUUCCCUUCAAA |
| siRNA 2224 | 2224 | UUGAAGGGAACAAUCAGAU | 5122 | AUCUGAUUGUUCCCUUCAA |
| siRNA 2225 | 2225 | UGAAGGGAACAAUCAGAUG | 5123 | CAUCUGAUUGUUCCCUUCA |
| siRNA 2226 | 2226 | GAAGGGAACAAUCAGAUGC | 5124 | GCAUCUGAUUGUUCCCUUC |
| siRNA 2227 | 2227 | AAGGGAACAAUCAGAUGCA | 5125 | UGCAUCUGAUUGUUCCCUU |
| siRNA 2228 | 2228 | AGGGAACAAUCAGAUGCAA | 5126 | UUGCAUCUGAUUGUUCCCU |
| siRNA 2229 | 2229 | GGGAACAAUCAGAUGCAAA | 5127 | UUUGCAUCUGAUUGUUCCC |
| siRNA 2230 | 2230 | GGAACAAUCAGAUGCAAAA | 5128 | UUUUGCAUCUGAUUGUUCC |
| siRNA 2231 | 2231 | GAACAAUCAGAUGCAAAAG | 5129 | CUUUUGCAUCUGAUUGUUC |
| siRNA 2232 | 2232 | AACAAUCAGAUGCAAAAGC | 5130 | GCUUUUGCAUCUGAUUGUU |
| siRNA 2233 | 2233 | ACAAUCAGAUGCAAAAGCU | 5131 | AGCUUUUGCAUCUGAUUGU |
| siRNA 2234 | 2234 | CAAUCAGAUGCAAAAGCUC | 5132 | GAGCUUUUGCAUCUGAUUG |
| siRNA 2235 | 2235 | AAUCAGAUGCAAAAGCUCU | 5133 | AGAGCUUUUGCAUCUGAUU |
| siRNA 2236 | 2236 | AUCAGAUGCAAAAGCUCUU | 5134 | AAGAGCUUUUGCAUCUGAU |
| siRNA 2237 | 2237 | UCAGAUGCAAAAGCUCUUG | 5135 | CAAGAGCUUUUGCAUCUGA |
| siRNA 2238 | 2238 | CAGAUGCAAAAGCUCUUGG | 5136 | CCAAGAGCUUUUGCAUCUG |
| siRNA 2239 | 2239 | AGAUGCAAAAGCUCUUGGG | 5137 | CCCAAGAGCUUUUGCAUCU |
| siRNA 2240 | 2240 | GAUGCAAAAGCUCUUGGGU | 5138 | ACCCAAGAGCUUUUGCAUC |
| siRNA 2241 | 2241 | AUGCAAAAGCUCUUGGGUG | 5139 | CACCCAAGAGCUUUUGCAU |
| siRNA 2242 | 2242 | UGCAAAAGCUCUUGGGUGU | 5140 | ACACCCAAGAGCUUUUGCA |
| siRNA 2243 | 2243 | GCAAAAGCUCUUGGGUGUU | 5141 | AACACCCAAGAGCUUUUGC |
| siRNA 2244 | 2244 | CAAAAGCUCUUGGGUGUUU | 5142 | AAACACCCAAGAGCUUUUG |
| siRNA 2245 | 2245 | AAAAGCUCUUGGGUGUUUA | 5143 | UAAACACCCAAGAGCUUUU |
| siRNA 2246 | 2246 | AAAGCUCUUGGGUGUUUAU | 5144 | AUAAACACCCAAGAGCUUU |
| siRNA 2247 | 2247 | AAGCUCUUGGGUGUUUAUU | 5145 | AAUAAACACCCAAGAGCUU |
| siRNA 2248 | 2248 | AGCUCUUGGGUGUUUAUUU | 5146 | AAAUAAACACCCAAGAGCU |
| siRNA 2249 | 2249 | GCUCUUGGGUGUUUAUUUA | 5147 | UAAAUAAACACCCAAGAGC |
| siRNA 2250 | 2250 | CUCUUGGGUGUUUAUUUAA | 5148 | UUAAAUAAACACCCAAGAG |
| siRNA 2251 | 2251 | UCUUGGGUGUUUAUUUAAA | 5149 | UUUAAAUAAACACCCAAGA |
| siRNA 2252 | 2252 | CUUGGGUGUUUAUUUAAAA | 5150 | UUUUAAAUAAACACCCAAG |
| siRNA 2253 | 2253 | UUGGGUGUUUAUUUAAAAU | 5151 | AUUUUAAAUAAACACCCAA |
| siRNA 2254 | 2254 | UGGGUGUUUAUUUAAAAUA | 5152 | UAUUUUAAAUAAACACCCA |
| siRNA 2255 | 2255 | GGGUGUUUAUUUAAAAUAC | 5153 | GUAUUUUAAAUAAACACCC |
| siRNA 2256 | 2256 | GGUGUUUAUUUAAAAUACU | 5154 | AGUAUUUUAAAUAAACACC |
| siRNA 2257 | 2257 | GUGUUUAUUUAAAAUACUA | 5155 | UAGUAUUUUAAAUAAACAC |
| siRNA 2258 | 2258 | UGUUUAUUUAAAAUACUAG | 5156 | CUAGUAUUUUAAAUAAACA |
| siRNA 2259 | 2259 | GUUUAUUUAAAAUACUAGU | 5157 | ACUAGUAUUUUAAAUAAAC |
| siRNA 2260 | 2260 | UUUAUUUAAAAUACUAGUG | 5158 | CACUAGUAUUUUAAAUAAA |
| siRNA 2261 | 2261 | UUAUUUAAAAUACUAGUGU | 5159 | ACACUAGUAUUUUAAAUAA |
| siRNA 2262 | 2262 | UAUUUAAAAUACUAGUGUC | 5160 | GACACUAGUAUUUUAAAUA |
| siRNA 2263 | 2263 | AUUUAAAAUACUAGUGUCA | 5161 | UGACACUAGUAUUUUAAAU |
| siRNA 2264 | 2264 | UUUAAAAUACUAGUGUCAC | 5162 | GUGACACUAGUAUUUUAAA |
| siRNA 2265 | 2265 | UUAAAAUACUAGUGUCACU | 5163 | AGUGACACUAGUAUUUUAA |
| siRNA 2266 | 2266 | UAAAAUACUAGUGUCACUU | 5164 | AAGUGACACUAGUAUUUUA |
| siRNA 2267 | 2267 | AAAAUACUAGUGUCACUUU | 5165 | AAAGUGACACUAGUAUUUU |
| siRNA 2268 | 2268 | AAAUACUAGUGUCACUUUC | 5166 | GAAAGUGACACUAGUAUUU |
| siRNA 2269 | 2269 | AAUACUAGUGUCACUUUCU | 5167 | AGAAAGUGACACUAGUAUU |
| siRNA 2270 | 2270 | AUACUAGUGUCACUUUCUG | 5168 | CAGAAAGUGACACUAGUAU |
| siRNA 2271 | 2271 | UACUAGUGUCACUUUCUGA | 5169 | UCAGAAAGUGACACUAGUA |
| siRNA 2272 | 2272 | ACUAGUGUCACUUUCUGAG | 5170 | CUCAGAAAGUGACACUAGU |
| siRNA 2273 | 2273 | CUAGUGUCACUUUCUGAGU | 5171 | ACUCAGAAAGUGACACUAG |
| siRNA 2274 | 2274 | UAGUGUCACUUUCUGAGUA | 5172 | UACUCAGAAAGUGACACUA |
| siRNA 2275 | 2275 | AGUGUCACUUUCUGAGUAC | 5173 | GUACUCAGAAAGUGACACU |
| siRNA 2276 | 2276 | GUGUCACUUUCUGAGUACC | 5174 | GGUACUCAGAAAGUGACAC |
| SiRNA 2277 | 2277 | UGUCACUUUCUGAGUACCC | 5175 | GGGUACUCAGAAAGUGACA |
| siRNA 2278 | 2278 | GUCACUUUCUGAGUACCCG | 5176 | CGGGUACUCAGAAAGUGAC |
| siRNA 2279 | 2279 | UCACUUUCUGAGUACCCGC | 5177 | GCGGGUACUCAGAAAGUGA |
| siRNA 2280 | 2280 | CACUUUCUGAGUACCCGCC | 5178 | GGCGGGUACUCAGAAAGUG |
| siRNA 2281 | 2281 | ACUUUCUGAGUACCCGCCG | 5179 | CGGCGGGUACUCAGAAAGU |
| siRNA 2282 | 2282 | CUUUCUGAGUACCCGCCGC | 5180 | GCGGCGGGUACUCAGAAAG |
| siRNA 2283 | 2283 | UUUCUGAGUACCCGCCGCU | 5181 | AGCGGCGGGUACUCAGAAA |
| siRNA 2284 | 2284 | UUCUGAGUACCCGCCGCUU | 5182 | AAGCGGCGGGUACUCAGAA |
| siRNA 2285 | 2285 | UCUGAGUACCCGCCGCUUC | 5183 | GAAGCGGCGGGUACUCAGA |
| siRNA 2286 | 2286 | CUGAGUACCCGCCGCUUCA | 5184 | UGAAGCGGCGGGUACUCAG |
| siRNA 2287 | 2287 | UGAGUACCCGCCGCUUCAC | 5185 | GUGAAGCGGCGGGUACUCA |
| siRNA 2288 | 2288 | GAGUACCCGCCGCUUCACA | 5186 | UGUGAAGCGGCGGGUACUC |
| siRNA 2289 | 2289 | AGUACCCGCCGCUUCACAG | 5187 | CUGUGAAGCGGCGGGUACU |
| siRNA 2290 | 2290 | GUACCCGCCGCUUCACAGG | 5188 | CCUGUGAAGCGGCGGGUAC |
| siRNA 2291 | 2291 | UACCCGCCGCUUCACAGGC | 5189 | GCCUGUGAAGCGGCGGGUA |
| siRNA 2292 | 2292 | ACCCGCCGCUUCACAGGCU | 5190 | AGCCUGUGAAGCGGCGGGU |
| siRNA 2293 | 2293 | CCCGCCGCUUCACAGGCUG | 5191 | CAGCCUGUGAAGCGGCGGG |
| siRNA 2294 | 2294 | CCGCCGCUUCACAGGCUGA | 5192 | UCAGCCUGUGAAGCGGCGG |
| siRNA 2295 | 2295 | CGCCGCUUCACAGGCUGAG | 5193 | CUCAGCCUGUGAAGCGGCG |
| siRNA 2296 | 2296 | GCCGCUUCACAGGCUGAGU | 5194 | ACUCAGCCUGUGAAGCGGC |
| siRNA 2297 | 2297 | CCGCUUCACAGGCUGAGUC | 5195 | GACUCAGCCUGUGAAGCGG |
| siRNA 2298 | 2298 | CGCUUCACAGGCUGAGUCC | 5196 | GGACUCAGCCUGUGAAGCG |
| siRNA 2299 | 2299 | GCUUCACAGGCUGAGUCCA | 5197 | UGGACUCAGCCUGUGAAGC |
| siRNA 2300 | 2300 | CUUCACAGGCUGAGUCCAG | 5198 | CUGGACUCAGCCUGUGAAG |
| siRNA 2301 | 2301 | UUCACAGGCUGAGUCCAGG | 5199 | CCUGGACUCAGCCUGUGAA |
| siRNA 2302 | 2302 | UCACAGGCUGAGUCCAGGC | 5200 | GCCUGGACUCAGCCUGUGA |
| siRNA 2303 | 2303 | CACAGGCUGAGUCCAGGCC | 5201 | GGCCUGGACUCAGCCUGUG |
| siRNA 2304 | 2304 | ACAGGCUGAGUCCAGGCCU | 5202 | AGGCCUGGACUCAGCCUGU |
| siRNA 2305 | 2305 | CAGGCUGAGUCCAGGCCUG | 5203 | CAGGCCUGGACUCAGCCUG |
| siRNA 2306 | 2306 | AGGCUGAGUCCAGGCCUGU | 5204 | ACAGGCCUGGACUCAGCCU |
| siRNA 2307 | 2307 | GGCUGAGUCCAGGCCUGUG | 5205 | CACAGGCCUGGACUCAGCC |
| siRNA 2308 | 2308 | GCUGAGUCCAGGCCUGUGU | 5206 | ACACAGGCCUGGACUCAGC |
| siRNA 2309 | 2309 | CUGAGUCCAGGCCUGUGUG | 5207 | CACACAGGCCUGGACUCAG |
| siRNA 2310 | 2310 | UGAGUCCAGGCCUGUGUGC | 5208 | GCACACAGGCCUGGACUCA |
| siRNA 2311 | 2311 | GAGUCCAGGCCUGUGUGCU | 5209 | AGCACACAGGCCUGGACUC |
| siRNA 2312 | 2312 | AGUCCAGGCCUGUGUGCUU | 5210 | AAGCACACAGGCCUGGACU |
| siRNA 2313 | 2313 | GUCCAGGCCUGUGUGCUUU | 5211 | AAAGCACACAGGCCUGGAC |
| siRNA 2314 | 2314 | UCCAGGCCUGUGUGCUUUG | 5212 | CAAAGCACACAGGCCUGGA |
| siRNA 2315 | 2315 | CCAGGCCUGUGUGCUUUGU | 5213 | ACAAAGCACACAGGCCUGG |
| siRNA 2316 | 2316 | CAGGCCUGUGUGCUUUGUA | 5214 | UACAAAGCACACAGGCCUG |
| siRNA 2317 | 2317 | AGGCCUGUGUGCUUUGUAG | 5215 | CUACAAAGCACACAGGCCU |
| siRNA 2318 | 2318 | GGCCUGUGUGCUUUGUAGA | 5216 | UCUACAAAGCACACAGGCC |
| siRNA 2319 | 2319 | GCCUGUGUGCUUUGUAGAG | 5217 | CUCUACAAAGCACACAGGC |
| siRNA 2320 | 2320 | CCUGUGUGCUUUGUAGAGC | 5218 | GCUCUACAAAGCACACAGG |
| siRNA 2321 | 2321 | CUGUGUGCUUUGUAGAGCC | 5219 | GGCUCUACAAAGCACACAG |
| siRNA 2322 | 2322 | UGUGUGCUUUGUAGAGCCA | 5220 | UGGCUCUACAAAGCACACA |
| siRNA 2323 | 2323 | GUGUGCUUUGUAGAGCCAG | 5221 | CUGGCUCUACAAAGCACAC |
| siRNA 2324 | 2324 | UGUGCUUUGUAGAGCCAGC | 5222 | GCUGGCUCUACAAAGCACA |
| siRNA 2325 | 2325 | GUGCUUUGUAGAGCCAGCU | 5223 | AGCUGGCUCUACAAAGCAC |
| siRNA 2326 | 2326 | UGCUUUGUAGAGCCAGCUG | 5224 | CAGCUGGCUCUACAAAGCA |
| siRNA 2327 | 2327 | GCUUUGUAGAGCCAGCUGC | 5225 | GCAGCUGGCUCUACAAAGC |
| siRNA 2328 | 2328 | CUUUGUAGAGCCAGCUGCU | 5226 | AGCAGCUGGCUCUACAAAG |
| siRNA 2329 | 2329 | UUUGUAGAGCCAGCUGCUU | 5227 | AAGCAGCUGGCUCUACAAA |
| siRNA 2330 | 2330 | UUGUAGAGCCAGCUGCUUG | 5228 | CAAGCAGCUGGCUCUACAA |
| siRNA 2331 | 2331 | UGUAGAGCCAGCUGCUUGC | 5229 | GCAAGCAGCUGGCUCUACA |
| siRNA 2332 | 2332 | GUAGAGCCAGCUGCUUGCU | 5230 | AGCAAGCAGCUGGCUCUAC |
| siRNA 2333 | 2333 | UAGAGCCAGCUGCUUGCUC | 5231 | GAGCAAGCAGCUGGCUCUA |
| siRNA 2334 | 2334 | AGAGCCAGCUGCUUGCUCA | 5232 | UGAGCAAGCAGCUGGCUCU |
| siRNA 2335 | 2335 | GAGCCAGCUGCUUGCUCAC | 5233 | GUGAGCAAGCAGCUGGCUC |
| siRNA 2336 | 2336 | AGCCAGCUGCUUGCUCACA | 5234 | UGUGAGCAAGCAGCUGGCU |
| siRNA 2337 | 2337 | GCCAGCUGCUUGCUCACAG | 5235 | CUGUGAGCAAGCAGCUGGC |
| siRNA 2338 | 2338 | CCAGCUGCUUGCUCACAGC | 5236 | GCUGUGAGCAAGCAGCUGG |
| siRNA 2339 | 2339 | CAGCUGCUUGCUCACAGCC | 5237 | GGCUGUGAGCAAGCAGCUG |
| siRNA 2340 | 2340 | AGCUGCUUGCUCACAGCCA | 5238 | UGGCUGUGAGCAAGCAGCU |
| siRNA 2341 | 2341 | GCUGCUUGCUCACAGCCAC | 5239 | GUGGCUGUGAGCAAGCAGC |
| siRNA 2342 | 2342 | CUGCUUGCUCACAGCCACA | 5240 | UGUGGCUGUGAGCAAGCAG |
| siRNA 2343 | 2343 | UGCUUGCUCACAGCCACAU | 5241 | AUGUGGCUGUGAGCAAGCA |
| siRNA 2344 | 2344 | GCUUGCUCACAGCCACAUU | 5242 | AAUGUGGCUGUGAGCAAGC |
| siRNA 2345 | 2345 | CUUGCUCACAGCCACAUUU | 5243 | AAAUGUGGCUGUGAGCAAG |
| siRNA 2346 | 2346 | UUGCUCACAGCCACAUUUC | 5244 | GAAAUGUGGCUGUGAGCAA |
| siRNA 2347 | 2347 | UGCUCACAGCCACAUUUCC | 5245 | GGAAAUGUGGCUGUGAGCA |
| siRNA 2348 | 2348 | GCUCACAGCCACAUUUCCA | 5246 | UGGAAAUGUGGCUGUGAGC |
| siRNA 2349 | 2349 | CUCACAGCCACAUUUCCAU | 5247 | AUGGAAAUGUGGCUGUGAG |
| siRNA 2350 | 2350 | UCACAGCCACAUUUCCAUU | 5248 | AAUGGAAAUGUGGCUGUGA |
| siRNA 2351 | 2351 | CACAGCCACAUUUCCAUUU | 5249 | AAAUGGAAAUGUGGCUGUG |
| siRNA 2352 | 2352 | ACAGCCACAUUUCCAUUUG | 5250 | CAAAUGGAAAUGUGGCUGU |
| siRNA 2353 | 2353 | CAGCCACAUUUCCAUUUGC | 5251 | GCAAAUGGAAAUGUGGCUG |
| siRNA 2354 | 2354 | AGCCACAUUUCCAUUUGCA | 5252 | UGCAAAUGGAAAUGUGGCU |
| siRNA 2355 | 2355 | GCCACAUUUCCAUUUGCAU | 5253 | AUGCAAAUGGAAAUGUGGC |
| siRNA 2356 | 2356 | CCACAUUUCCAUUUGCAUC | 5254 | GAUGCAAAUGGAAAUGUGG |
| siRNA 2357 | 2357 | CACAUUUCCAUUUGCAUCA | 5255 | UGAUGCAAAUGGAAAUGUG |
| siRNA 2358 | 2358 | ACAUUUCCAUUUGCAUCAU | 5256 | AUGAUGCAAAUGGAAAUGU |
| siRNA 2359 | 2359 | CAUUUCCAUUUGCAUCAUU | 5257 | AAUGAUGCAAAUGGAAAUG |
| siRNA 2360 | 2360 | AUUUCCAUUUGCAUCAUUA | 5258 | UAAUGAUGCAAAUGGAAAU |
| siRNA 2361 | 2361 | UUUCCAUUUGCAUCAUUAC | 5259 | GUAAUGAUGCAAAUGGAAA |
| siRNA 2362 | 2362 | UUCCAUUUGCAUCAUUACU | 5260 | AGUAAUGAUGCAAAUGGAA |
| siRNA 2363 | 2363 | UCCAUUUGCAUCAUUACUG | 5261 | CAGUAAUGAUGCAAAUGGA |
| siRNA 2364 | 2364 | CCAUUUGCAUCAUUACUGC | 5262 | GCAGUAAUGAUGCAAAUGG |
| siRNA 2365 | 2365 | CAUUUGCAUCAUUACUGCC | 5263 | GGCAGUAAUGAUGCAAAUG |
| siRNA 2366 | 2366 | AUUUGCAUCAUUACUGCCU | 5264 | AGGCAGUAAUGAUGCAAAU |
| siRNA 2367 | 2367 | UUUGCAUCAUUACUGCCUU | 5265 | AAGGCAGUAAUGAUGCAAA |
| siRNA 2368 | 2368 | UUGCAUCAUUACUGCCUUC | 5266 | GAAGGCAGUAAUGAUGCAA |
| siRNA 2369 | 2369 | UGCAUCAUUACUGCCUUCA | 5267 | UGAAGGCAGUAAUGAUGCA |
| siRNA 2370 | 2370 | GCAUCAUUACUGCCUUCAC | 5268 | GUGAAGGCAGUAAUGAUGC |
| siRNA 2371 | 2371 | CAUCAUUACUGCCUUCACC | 5269 | GGUGAAGGCAGUAAUGAUG |
| siRNA 2372 | 2372 | AUCAUUACUGCCUUCACCU | 5270 | AGGUGAAGGCAGUAAUGAU |
| siRNA 2373 | 2373 | UCAUUACUGCCUUCACCUG | 5271 | CAGGUGAAGGCAGUAAUGA |
| siRNA 2374 | 2374 | CAUUACUGCCUUCACCUGC | 5272 | GCAGGUGAAGGCAGUAAUG |
| siRNA 2375 | 2375 | AUUACUGCCUUCACCUGCA | 5273 | UGCAGGUGAAGGCAGUAAU |
| siRNA 2376 | 2376 | UUACUGCCUUCACCUGCAU | 5274 | AUGCAGGUGAAGGCAGUAA |
| siRNA 2377 | 2377 | UACUGCCUUCACCUGCAUA | 5275 | UAUGCAGGUGAAGGCAGUA |
| siRNA 2378 | 2378 | ACUGCCUUCACCUGCAUAG | 5276 | CUAUGCAGGUGAAGGCAGU |
| siRNA 2379 | 2379 | CUGCCUUCACCUGCAUAGU | 5277 | ACUAUGCAGGUGAAGGCAG |
| siRNA 2380 | 2380 | UGCCUUCACCUGCAUAGUC | 5278 | GACUAUGCAGGUGAAGGCA |
| siRNA 2381 | 2381 | GCCUUCACCUGCAUAGUCA | 5279 | UGACUAUGCAGGUGAAGGC |
| siRNA 2382 | 2382 | CCUUCACCUGCAUAGUCAC | 5280 | GUGACUAUGCAGGUGAAGG |
| siRNA 2383 | 2383 | CUUCACCUGCAUAGUCACU | 5281 | AGUGACUAUGCAGGUGAAG |
| siRNA 2384 | 2384 | UUCACCUGCAUAGUCACUC | 5282 | GAGUGACUAUGCAGGUGAA |
| siRNA 2385 | 2385 | UCACCUGCAUAGUCACUCU | 5283 | AGAGUGACUAUGCAGGUGA |
| siRNA 2386 | 2386 | CACCUGCAUAGUCACUCUU | 5284 | AAGAGUGACUAUGCAGGUG |
| siRNA 2387 | 2387 | ACCUGCAUAGUCACUCUUU | 5285 | AAAGAGUGACUAUGCAGGU |
| siRNA 2388 | 2388 | CCUGCAUAGUCACUCUUUU | 5286 | AAAAGAGUGACUAUGCAGG |
| siRNA 2389 | 2389 | CUGCAUAGUCACUCUUUUG | 5287 | CAAAAGAGUGACUAUGCAG |
| siRNA 2390 | 2390 | UGCAUAGUCACUCUUUUGA | 5288 | UCAAAAGAGUGACUAUGCA |
| siRNA 2391 | 2391 | GCAUAGUCACUCUUUUGAU | 5289 | AUCAAAAGAGUGACUAUGC |
| siRNA 2392 | 2392 | CAUAGUCACUCUUUUGAUG | 5290 | CAUCAAAAGAGUGACUAUG |
| siRNA 2393 | 2393 | AUAGUCACUCUUUUGAUGC | 5291 | GCAUCAAAAGAGUGACUAU |
| siRNA 2394 | 2394 | UAGUCACUCUUUUGAUGCU | 5292 | AGCAUCAAAAGAGUGACUA |
| siRNA 2395 | 2395 | AGUCACUCUUUUGAUGCUG | 5293 | CAGCAUCAAAAGAGUGACU |
| siRNA 2396 | 2396 | GUCACUCUUUUGAUGCUGG | 5294 | CCAGCAUCAAAAGAGUGAC |
| siRNA 2397 | 2397 | UCACUCUUUUGAUGCUGGG | 5295 | CCCAGCAUCAAAAGAGUGA |
| siRNA 2398 | 2398 | CACUCUUUUGAUGCUGGGG | 5296 | CCCCAGCAUCAAAAGAGUG |
| siRNA 2399 | 2399 | ACUCUUUUGAUGCUGGGGA | 5297 | UCCCCAGCAUCAAAAGAGU |
| siRNA 2400 | 2400 | CUCUUUUGAUGCUGGGGAA | 5298 | UUCCCCAGCAUCAAAAGAG |
| siRNA 2401 | 2401 | UCUUUUGAUGCUGGGGAAC | 5299 | GUUCCCCAGCAUCAAAAGA |
| siRNA 2402 | 2402 | CUUUUGAUGCUGGGGAACC | 5300 | GGUUCCCCAGCAUCAAAAG |
| siRNA 2403 | 2403 | UUUUGAUGCUGGGGAACCA | 5301 | UGGUUCCCCAGCAUCAAAA |
| siRNA 2404 | 2404 | UUUGAUGCUGGGGAACCAA | 5302 | UUGGUUCCCCAGCAUCAAA |
| siRNA 2405 | 2405 | UUGAUGCUGGGGAACCAAA | 5303 | UUUGGUUCCCCAGCAUCAA |
| siRNA 2406 | 2406 | UGAUGCUGGGGAACCAAAA | 5304 | UUUUGGUUCCCCAGCAUCA |
| siRNA 2407 | 2407 | GAUGCUGGGGAACCAAAAU | 5305 | AUUUUGGUUCCCCAGCAUC |
| siRNA 2408 | 2408 | AUGCUGGGGAACCAAAAUG | 5306 | CAUUUUGGUUCCCCAGCAU |
| siRNA 2409 | 2409 | UGCUGGGGAACCAAAAUGG | 5307 | CCAUUUUGGUUCCCCAGCA |
| siRNA 2410 | 2410 | GCUGGGGAACCAAAAUGGU | 5308 | ACCAUUUUGGUUCCCCAGC |
| siRNA 2411 | 2411 | CUGGGGAACCAAAAUGGUG | 5309 | CACCAUUUUGGUUCCCCAG |
| siRNA 2412 | 2412 | UGGGGAACCAAAAUGGUGA | 5310 | UCACCAUUUUGGUUCCCCA |
| siRNA 2413 | 2413 | GGGGAACCAAAAUGGUGAU | 5311 | AUCACCAUUUUGGUUCCCC |
| siRNA 2414 | 2414 | GGGAACCAAAAUGGUGAUG | 5312 | CAUCACCAUUUUGGUUCCC |
| siRNA 2415 | 2415 | GGAACCAAAAUGGUGAUGA | 5313 | UCAUCACCAUUUUGGUUCC |
| siRNA 2416 | 2416 | GAACCAAAAUGGUGAUGAU | 5314 | AUCAUCACCAUUUUGGUUC |
| siRNA 2417 | 2417 | AACCAAAAUGGUGAUGAUA | 5315 | UAUCAUCACCAUUUUGGUU |
| siRNA 2418 | 2418 | ACCAAAAUGGUGAUGAUAU | 5316 | AUAUCAUCACCAUUUUGGU |
| siRNA 2419 | 2419 | CCAAAAUGGUGAUGAUAUA | 5317 | UAUAUCAUCACCAUUUUGG |
| siRNA 2420 | 2420 | CAAAAUGGUGAUGAUAUAU | 5318 | AUAUAUCAUCACCAUUUUG |
| siRNA 2421 | 2421 | AAAAUGGUGAUGAUAUAUA | 5319 | UAUAUAUCAUCACCAUUUU |
| siRNA 2422 | 2422 | AAAUGGUGAUGAUAUAUAG | 5320 | CUAUAUAUCAUCACCAUUU |
| siRNA 2423 | 2423 | AAUGGUGAUGAUAUAUAGA | 5321 | UCUAUAUAUCAUCACCAUU |
| siRNA 2424 | 2424 | AUGGUGAUGAUAUAUAGAC | 5322 | GUCUAUAUAUCAUCACCAU |
| siRNA 2425 | 2425 | UGGUGAUGAUAUAUAGACU | 5323 | AGUCUAUAUAUCAUCACCA |
| siRNA 2426 | 2426 | GGUGAUGAUAUAUAGACUU | 5324 | AAGUCUAUAUAUCAUCACC |
| siRNA 2427 | 2427 | GUGAUGAUAUAUAGACUUU | 5325 | AAAGUCUAUAUAUCAUCAC |
| siRNA 2428 | 2428 | UGAUGAUAUAUAGACUUUA | 5326 | UAAAGUCUAUAUAUCAUCA |
| siRNA 2429 | 2429 | GAUGAUAUAUAGACUUUAU | 5327 | AUAAAGUCUAUAUAUCAUC |
| siRNA 2430 | 2430 | AUGAUAUAUAGACUUUAUG | 5328 | CAUAAAGUCUAUAUAUCAU |
| siRNA 2431 | 2431 | UGAUAUAUAGACUUUAUGU | 5329 | ACAUAAAGUCUAUAUAUCA |
| siRNA 2432 | 2432 | GAUAUAUAGACUUUAUGUA | 5330 | UACAUAAAGUCUAUAUAUC |
| siRNA 2433 | 2433 | AUAUAUAGACUUUAUGUAU | 5331 | AUACAUAAAGUCUAUAUAU |
| siRNA 2434 | 2434 | UAUAUAGACUUUAUGUAUA | 5332 | UAUACAUAAAGUCUAUAUA |
| siRNA 2435 | 2435 | AUAUAGACUUUAUGUAUAG | 5333 | CUAUACAUAAAGUCUAUAU |
| siRNA 2436 | 2436 | UAUAGACUUUAUGUAUAGC | 5334 | GCUAUACAUAAAGUCUAUA |
| siRNA 2437 | 2437 | AUAGACUUUAUGUAUAGCC | 5335 | GGCUAUACAUAAAGUCUAU |
| siRNA 2438 | 2438 | UAGACUUUAUGUAUAGCCA | 5336 | UGGCUAUACAUAAAGUCUA |
| siRNA 2439 | 2439 | AGACUUUAUGUAUAGCCAC | 5337 | GUGGCUAUACAUAAAGUCU |
| siRNA 2440 | 2440 | GACUUUAUGUAUAGCCACA | 5338 | UGUGGCUAUACAUAAAGUC |
| siRNA 2441 | 2441 | ACUUUAUGUAUAGCCACAG | 5339 | CUGUGGCUAUACAUAAAGU |
| siRNA 2442 | 2442 | CUUUAUGUAUAGCCACAGU | 5340 | ACUGUGGCUAUACAUAAAG |
| siRNA 2443 | 2443 | UUUAUGUAUAGCCACAGUU | 5341 | AACUGUGGCUAUACAUAAA |
| siRNA 2444 | 2444 | UUAUGUAUAGCCACAGUUC | 5342 | GAACUGUGGCUAUACAUAA |
| siRNA 2445 | 2445 | UAUGUAUAGCCACAGUUCA | 5343 | UGAACUGUGGCUAUACAUA |
| siRNA 2446 | 2446 | AUGUAUAGCCACAGUUCAU | 5344 | AUGAACUGUGGCUAUACAU |
| siRNA 2447 | 2447 | UGUAUAGCCACAGUUCAUC | 5345 | GAUGAACUGUGGCUAUACA |
| siRNA 2448 | 2448 | GUAUAGCCACAGUUCAUCC | 5346 | GGAUGAACUGUGGCUAUAC |
| siRNA 2449 | 2449 | UAUAGCCACAGUUCAUCCC | 5347 | GGGAUGAACUGUGGCUAUA |
| siRNA 2450 | 2450 | AUAGCCACAGUUCAUCCCC | 5348 | GGGGAUGAACUGUGGCUAU |
| siRNA 2451 | 2451 | UAGCCACAGUUCAUCCCCA | 5349 | UGGGGAUGAACUGUGGCUA |
| siRNA 2452 | 2452 | AGCCACAGUUCAUCCCCAA | 5350 | UUGGGGAUGAACUGUGGCU |
| siRNA 2453 | 2453 | GCCACAGUUCAUCCCCAAC | 5351 | GUUGGGGAUGAACUGUGGC |
| siRNA 2454 | 2454 | CCACAGUUCAUCCCCAACC | 5352 | GGUUGGGGAUGAACUGUGG |
| siRNA 2455 | 2455 | CACAGUUCAUCCCCAACCC | 5353 | GGGUUGGGGAUGAACUGUG |
| siRNA 2456 | 2456 | ACAGUUCAUCCCCAACCCU | 5354 | AGGGUUGGGGAUGAACUGU |
| siRNA 2457 | 2457 | CAGUUCAUCCCCAACCCUA | 5355 | UAGGGUUGGGGAUGAACUG |
| siRNA 2458 | 2458 | AGUUCAUCCCCAACCCUAG | 5356 | CUAGGGUUGGGGAUGAACU |
| siRNA 2459 | 2459 | GUUCAUCCCCAACCCUAGU | 5357 | ACUAGGGUUGGGGAUGAAC |
| siRNA 2460 | 2460 | UUCAUCCCCAACCCUAGUC | 5358 | GACUAGGGUUGGGGAUGAA |
| siRNA 2461 | 2461 | UCAUCCCCAACCCUAGUCU | 5359 | AGACUAGGGUUGGGGAUGA |
| siRNA 2462 | 2462 | CAUCCCCAACCCUAGUCUU | 5360 | AAGACUAGGGUUGGGGAUG |
| siRNA 2463 | 2463 | AUCCCCAACCCUAGUCUUC | 5361 | GAAGACUAGGGUUGGGGAU |
| siRNA 2464 | 2464 | UCCCCAACCCUAGUCUUCG | 5362 | CGAAGACUAGGGUUGGGGA |
| siRNA 2465 | 2465 | CCCCAACCCUAGUCUUCGA | 5363 | UCGAAGACUAGGGUUGGGG |
| siRNA 2466 | 2466 | CCCAACCCUAGUCUUCGAA | 5364 | UUCGAAGACUAGGGUUGGG |
| siRNA 2467 | 2467 | CCAACCCUAGUCUUCGAAA | 5365 | UUUCGAAGACUAGGGUUGG |
| siRNA 2468 | 2468 | CAACCCUAGUCUUCGAAAU | 5366 | AUUUCGAAGACUAGGGUUG |
| siRNA 2469 | 2469 | AACCCUAGUCUUCGAAAUG | 5367 | CAUUUCGAAGACUAGGGUU |
| siRNA 2470 | 2470 | ACCCUAGUCUUCGAAAUGU | 5368 | ACAUUUCGAAGACUAGGGU |
| siRNA 2471 | 2471 | CCCUAGUCUUCGAAAUGUU | 5369 | AACAUUUCGAAGACUAGGG |
| siRNA 2472 | 2472 | CCUAGUCUUCGAAAUGUUA | 5370 | UAACAUUUCGAAGACUAGG |
| siRNA 2473 | 2473 | CUAGUCUUCGAAAUGUUAA | 5371 | UUAACAUUUCGAAGACUAG |
| siRNA 2474 | 2474 | UAGUCUUCGAAAUGUUAAU | 5372 | AUUAACAUUUCGAAGACUA |
| siRNA 2475 | 2475 | AGUCUUCGAAAUGUUAAUA | 5373 | UAUUAACAUUUCGAAGACU |
| siRNA 2476 | 2476 | GUCUUCGAAAUGUUAAUAU | 5374 | AUAUUAACAUUUCGAAGAC |
| siRNA 2477 | 2477 | UCUUCGAAAUGUUAAUAUU | 5375 | AAUAUUAACAUUUCGAAGA |
| siRNA 2478 | 2478 | CUUCGAAAUGUUAAUAUUU | 5376 | AAAUAUUAACAUUUCGAAG |
| siRNA 2479 | 2479 | UUCGAAAUGUUAAUAUUUG | 5377 | CAAAUAUUAACAUUUCGAA |
| siRNA 2480 | 2480 | UCGAAAUGUUAAUAUUUGA | 5378 | UCAAAUAUUAACAUUUCGA |
| siRNA 2481 | 2481 | CGAAAUGUUAAUAUUUGAU | 5379 | AUCAAAUAUUAACAUUUCG |
| siRNA 2482 | 2482 | GAAAUGUUAAUAUUUGAUA | 5380 | UAUCAAAUAUUAACAUUUC |
| siRNA 2483 | 2483 | AAAUGUUAAUAUUUGAUAA | 5381 | UUAUCAAAUAUUAACAUUU |
| siRNA 2484 | 2484 | AAUGUUAAUAUUUGAUAAA | 5382 | UUUAUCAAAUAUUAACAUU |
| SIRNA 2485 | 2485 | AUGUUAAUAUUUGAUAAAU | 5383 | AUUUAUCAAAUAUUAACAU |
| siRNA 2486 | 2486 | UGUUAAUAUUUGAUAAAUC | 5384 | GAUUUAUCAAAUAUUAACA |
| siRNA 2487 | 2487 | GUUAAUAUUUGAUAAAUCU | 5385 | AGAUUUAUCAAAUAUUAAC |
| siRNA 2488 | 2488 | UUAAUAUUUGAUAAAUCUA | 5386 | UAGAUUUAUCAAAUAUUAA |
| siRNA 2489 | 2489 | UAAUAUUUGAUAAAUCUAG | 5387 | CUAGAUUUAUCAAAUAUUA |
| siRNA 2490 | 2490 | AAUAUUUGAUAAAUCUAGA | 5388 | UCUAGAUUUAUCAAAUAUU |
| siRNA 2491 | 2491 | AUAUUUGAUAAAUCUAGAA | 5389 | UUCUAGAUUUAUCAAAUAU |
| siRNA 2492 | 2492 | UAUUUGAUAAAUCUAGAAA | 5390 | UUUCUAGAUUUAUCAAAUA |
| siRNA 2493 | 2493 | AUUUGAUAAAUCUAGAAAA | 5391 | UUUUCUAGAUUUAUCAAAU |
| siRNA 2494 | 2494 | UUUGAUAAAUCUAGAAAAU | 5392 | AUUUUCUAGAUUUAUCAAA |
| siRNA 2495 | 2495 | UUGAUAAAUCUAGAAAAUG | 5393 | CAUUUUCUAGAUUUAUCAA |
| siRNA 2496 | 2496 | UGAUAAAUCUAGAAAAUGC | 5394 | GCAUUUUCUAGAUUUAUCA |
| siRNA 2497 | 2497 | GAUAAAUCUAGAAAAUGCA | 5395 | UGCAUUUUCUAGAUUUAUC |
| SIRNA 2498 | 2498 | AUAAAUCUAGAAAAUGCAU | 5396 | AUGCAUUUUCUAGAUUUAU |
| siRNA 2499 | 2499 | UAAAUCUAGAAAAUGCAUU | 5397 | AAUGCAUUUUCUAGAUUUA |
| siRNA 2500 | 2500 | AAAUCUAGAAAAUGCAUUC | 5398 | GAAUGCAUUUUCUAGAUUU |
| siRNA 2501 | 2501 | AAUCUAGAAAAUGCAUUCA | 5399 | UGAAUGCAUUUUCUAGAUU |
| siRNA 2502 | 2502 | AUCUAGAAAAUGCAUUCAU | 5400 | AUGAAUGCAUUUUCUAGAU |
| siRNA 2503 | 2503 | UCUAGAAAAUGCAUUCAUA | 5401 | UAUGAAUGCAUUUUCUAGA |
| siRNA 2504 | 2504 | CUAGAAAAUGCAUUCAUAC | 5402 | GUAUGAAUGCAUUUUCUAG |
| siRNA 2505 | 2505 | UAGAAAAUGCAUUCAUACA | 5403 | UGUAUGAAUGCAUUUUCUA |
| siRNA 2506 | 2506 | AGAAAAUGCAUUCAUACAA | 5404 | UUGUAUGAAUGCAUUUUCU |
| siRNA 2507 | 2507 | GAAAAUGCAUUCAUACAAU | 5405 | AUUGUAUGAAUGCAUUUUC |
| siRNA 2508 | 2508 | AAAAUGCAUUCAUACAAUU | 5406 | AAUUGUAUGAAUGCAUUUU |
| siRNA 2509 | 2509 | AAAUGCAUUCAUACAAUUA | 5407 | UAAUUGUAUGAAUGCAUUU |
| siRNA 2510 | 2510 | AAUGCAUUCAUACAAUUAC | 5408 | GUAAUUGUAUGAAUGCAUU |
| siRNA 2511 | 2511 | AUGCAUUCAUACAAUUACA | 5409 | UGUAAUUGUAUGAAUGCAU |
| siRNA 2512 | 2512 | UGCAUUCAUACAAUUACAG | 5410 | CUGUAAUUGUAUGAAUGCA |
| siRNA 2513 | 2513 | GCAUUCAUACAAUUACAGA | 5411 | UCUGUAAUUGUAUGAAUGC |
| siRNA 2514 | 2514 | CAUUCAUACAAUUACAGAA | 5412 | UUCUGUAAUUGUAUGAAUG |
| siRNA 2515 | 2515 | AUUCAUACAAUUACAGAAU | 5413 | AUUCUGUAAUUGUAUGAAU |
| siRNA 2516 | 2516 | UUCAUACAAUUACAGAAUU | 5414 | AAUUCUGUAAUUGUAUGAA |
| siRNA 2517 | 2517 | UCAUACAAUUACAGAAUUC | 5415 | GAAUUCUGUAAUUGUAUGA |
| siRNA 2518 | 2518 | CAUACAAUUACAGAAUUCA | 5416 | UGAAUUCUGUAAUUGUAUG |
| siRNA 2519 | 2519 | AUACAAUUACAGAAUUCAA | 5417 | UUGAAUUCUGUAAUUGUAU |
| siRNA 2520 | 2520 | UACAAUUACAGAAUUCAAA | 5418 | UUUGAAUUCUGUAAUUGUA |
| siRNA 2521 | 2521 | ACAAUUACAGAAUUCAAAU | 5419 | AUUUGAAUUCUGUAAUUGU |
| siRNA 2522 | 2522 | CAAUUACAGAAUUCAAAUA | 5420 | UAUUUGAAUUCUGUAAUUG |
| siRNA 2523 | 2523 | AAUUACAGAAUUCAAAUAU | 5421 | AUAUUUGAAUUCUGUAAUU |
| siRNA 2524 | 2524 | AUUACAGAAUUCAAAUAUU | 5422 | AAUAUUUGAAUUCUGUAAU |
| siRNA 2525 | 2525 | UUACAGAAUUCAAAUAUUG | 5423 | CAAUAUUUGAAUUCUGUAA |
| siRNA 2526 | 2526 | UACAGAAUUCAAAUAUUGC | 5424 | GCAAUAUUUGAAUUCUGUA |
| siRNA 2527 | 2527 | ACAGAAUUCAAAUAUUGCA | 5425 | UGCAAUAUUUGAAUUCUGU |
| siRNA 2528 | 2528 | CAGAAUUCAAAUAUUGCAA | 5426 | UUGCAAUAUUUGAAUUCUG |
| siRNA 2529 | 2529 | AGAAUUCAAAUAUUGCAAA | 5427 | UUUGCAAUAUUUGAAUUCU |
| siRNA 2530 | 2530 | GAAUUCAAAUAUUGCAAAA | 5428 | UUUUGCAAUAUUUGAAUUC |
| siRNA 2531 | 2531 | AAUUCAAAUAUUGCAAAAG | 5429 | CUUUUGCAAUAUUUGAAUU |
| siRNA 2532 | 2532 | AUUCAAAUAUUGCAAAAGG | 5430 | CCUUUUGCAAUAUUUGAAU |
| siRNA 2533 | 2533 | UUCAAAUAUUGCAAAAGGA | 5431 | UCCUUUUGCAAUAUUUGAA |
| siRNA 2534 | 2534 | UCAAAUAUUGCAAAAGGAU | 5432 | AUCCUUUUGCAAUAUUUGA |
| siRNA 2535 | 2535 | CAAAUAUUGCAAAAGGAUG | 5433 | CAUCCUUUUGCAAUAUUUG |
| siRNA 2536 | 2536 | AAAUAUUGCAAAAGGAUGU | 5434 | ACAUCCUUUUGCAAUAUUU |
| siRNA 2537 | 2537 | AAUAUUGCAAAAGGAUGUG | 5435 | CACAUCCUUUUGCAAUAUU |
| siRNA 2538 | 2538 | AUAUUGCAAAAGGAUGUGU | 5436 | ACACAUCCUUUUGCAAUAU |
| siRNA 2539 | 2539 | UAUUGCAAAAGGAUGUGUG | 5437 | CACACAUCCUUUUGCAAUA |
| siRNA 2540 | 2540 | AUUGCAAAAGGAUGUGUGU | 5438 | ACACACAUCCUUUUGCAAU |
| siRNA 2541 | 2541 | UUGCAAAAGGAUGUGUGUC | 5439 | GACACACAUCCUUUUGCAA |
| siRNA 2542 | 2542 | UGCAAAAGGAUGUGUGUCU | 5440 | AGACACACAUCCUUUUGCA |
| siRNA 2543 | 2543 | GCAAAAGGAUGUGUGUCUU | 5441 | AAGACACACAUCCUUUUGC |
| siRNA 2544 | 2544 | CAAAAGGAUGUGUGUCUUU | 5442 | AAAGACACACAUCCUUUUG |
| siRNA 2545 | 2545 | AAAAGGAUGUGUGUCUUUC | 5443 | GAAAGACACACAUCCUUUU |
| siRNA 2546 | 2546 | AAAGGAUGUGUGUCUUUCU | 5444 | AGAAAGACACACAUCCUUU |
| siRNA 2547 | 2547 | AAGGAUGUGUGUCUUUCUC | 5445 | GAGAAAGACACACAUCCUU |
| siRNA 2548 | 2548 | AGGAUGUGUGUCUUUCUCC | 5446 | GGAGAAAGACACACAUCCU |
| siRNA 2549 | 2549 | GGAUGUGUGUCUUUCUCCC | 5447 | GGGAGAAAGACACACAUCC |
| siRNA 2550 | 2550 | GAUGUGUGUCUUUCUCCCC | 5448 | GGGGAGAAAGACACACAUC |
| siRNA 2551 | 2551 | AUGUGUGUCUUUCUCCCCG | 5449 | CGGGGAGAAAGACACACAU |
| siRNA 2552 | 2552 | UGUGUGUCUUUCUCCCCGA | 5450 | UCGGGGAGAAAGACACACA |
| siRNA 2553 | 2553 | GUGUGUCUUUCUCCCCGAG | 5451 | CUCGGGGAGAAAGACACAC |
| siRNA 2554 | 2554 | UGUGUCUUUCUCCCCGAGC | 5452 | GCUCGGGGAGAAAGACACA |
| siRNA 2555 | 2555 | GUGUCUUUCUCCCCGAGCU | 5453 | AGCUCGGGGAGAAAGACAC |
| siRNA 2556 | 2556 | UGUCUUUCUCCCCGAGCUC | 5454 | GAGCUCGGGGAGAAAGACA |
| siRNA 2557 | 2557 | GUCUUUCUCCCCGAGCUCC | 5455 | GGAGCUCGGGGAGAAAGAC |
| siRNA 2558 | 2558 | UCUUUCUCCCCGAGCUCCC | 5456 | GGGAGCUCGGGGAGAAAGA |
| siRNA 2559 | 2559 | CUUUCUCCCCGAGCUCCCC | 5457 | GGGGAGCUCGGGGAGAAAG |
| siRNA 2560 | 2560 | UUUCUCCCCGAGCUCCCCU | 5458 | AGGGGAGCUCGGGGAGAAA |
| siRNA 2561 | 2561 | UUCUCCCCGAGCUCCCCUG | 5459 | CAGGGGAGCUCGGGGAGAA |
| siRNA 2562 | 2562 | UCUCCCCGAGCUCCCCUGU | 5460 | ACAGGGGAGCUCGGGGAGA |
| siRNA 2563 | 2563 | CUCCCCGAGCUCCCCUGUU | 5461 | AACAGGGGAGCUCGGGGAG |
| siRNA 2564 | 2564 | UCCCCGAGCUCCCCUGUUC | 5462 | GAACAGGGGAGCUCGGGGA |
| siRNA 2565 | 2565 | CCCCGAGCUCCCCUGUUCC | 5463 | GGAACAGGGGAGCUCGGGG |
| siRNA 2566 | 2566 | CCCGAGCUCCCCUGUUCCC | 5464 | GGGAACAGGGGAGCUCGGG |
| siRNA 2567 | 2567 | CCGAGCUCCCCUGUUCCCC | 5465 | GGGGAACAGGGGAGCUCGG |
| siRNA 2568 | 2568 | CGAGCUCCCCUGUUCCCCU | 5466 | AGGGGAACAGGGGAGCUCG |
| siRNA 2569 | 2569 | GAGCUCCCCUGUUCCCCUU | 5467 | AAGGGGAACAGGGGAGCUC |
| siRNA 2570 | 2570 | AGCUCCCCUGUUCCCCUUC | 5468 | GAAGGGGAACAGGGGAGCU |
| siRNA 2571 | 2571 | GCUCCCCUGUUCCCCUUCA | 5469 | UGAAGGGGAACAGGGGAGC |
| siRNA 2572 | 2572 | CUCCCCUGUUCCCCUUCAU | 5470 | AUGAAGGGGAACAGGGGAG |
| siRNA 2573 | 2573 | UCCCCUGUUCCCCUUCAUU | 5471 | AAUGAAGGGGAACAGGGGA |
| siRNA 2574 | 2574 | CCCCUGUUCCCCUUCAUUG | 5472 | CAAUGAAGGGGAACAGGGG |
| siRNA 2575 | 2575 | CCCUGUUCCCCUUCAUUGA | 5473 | UCAAUGAAGGGGAACAGGG |
| siRNA 2576 | 2576 | CCUGUUCCCCUUCAUUGAA | 5474 | UUCAAUGAAGGGGAACAGG |
| siRNA 2577 | 2577 | CUGUUCCCCUUCAUUGAAA | 5475 | UUUCAAUGAAGGGGAACAG |
| siRNA 2578 | 2578 | UGUUCCCCUUCAUUGAAAA | 5476 | UUUUCAAUGAAGGGGAACA |
| siRNA 2579 | 2579 | GUUCCCCUUCAUUGAAAAC | 5477 | GUUUUCAAUGAAGGGGAAC |
| siRNA 2580 | 2580 | UUCCCCUUCAUUGAAAACC | 5478 | GGUUUUCAAUGAAGGGGAA |
| siRNA 2581 | 2581 | UCCCCUUCAUUGAAAACCA | 5479 | UGGUUUUCAAUGAAGGGGA |
| siRNA 2582 | 2582 | CCCCUUCAUUGAAAACCAC | 5480 | GUGGUUUUCAAUGAAGGGG |
| SIRNA 2583 | 2583 | CCCUUCAUUGAAAACCACC | 5481 | GGUGGUUUUCAAUGAAGGG |
| siRNA 2584 | 2584 | CCUUCAUUGAAAACCACCA | 5482 | UGGUGGUUUUCAAUGAAGG |
| siRNA 2585 | 2585 | CUUCAUUGAAAACCACCAC | 5483 | GUGGUGGUUUUCAAUGAAG |
| siRNA 2586 | 2586 | UUCAUUGAAAACCACCACG | 5484 | CGUGGUGGUUUUCAAUGAA |
| siRNA 2587 | 2587 | UCAUUGAAAACCACCACGG | 5485 | CCGUGGUGGUUUUCAAUGA |
| siRNA 2588 | 2588 | CAUUGAAAACCACCACGGU | 5486 | ACCGUGGUGGUUUUCAAUG |
| siRNA 2589 | 2589 | AUUGAAAACCACCACGGUG | 5487 | CACCGUGGUGGUUUUCAAU |
| siRNA 2590 | 2590 | UUGAAAACCACCACGGUGC | 5488 | GCACCGUGGUGGUUUUCAA |
| siRNA 2591 | 2591 | UGAAAACCACCACGGUGCC | 5489 | GGCACCGUGGUGGUUUUCA |
| siRNA 2592 | 2592 | GAAAACCACCACGGUGCCA | 5490 | UGGCACCGUGGUGGUUUUC |
| siRNA 2593 | 2593 | AAAACCACCACGGUGCCAU | 5491 | AUGGCACCGUGGUGGUUUU |
| siRNA 2594 | 2594 | AAACCACCACGGUGCCAUC | 5492 | GAUGGCACCGUGGUGGUUU |
| siRNA 2595 | 2595 | AACCACCACGGUGCCAUCU | 5493 | AGAUGGCACCGUGGUGGUU |
| siRNA 2596 | 2596 | ACCACCACGGUGCCAUCUC | 5494 | GAGAUGGCACCGUGGUGGU |
| siRNA 2597 | 2597 | CCACCACGGUGCCAUCUCU | 5495 | AGAGAUGGCACCGUGGUGG |
| siRNA 2598 | 2598 | CACCACGGUGCCAUCUCUU | 5496 | AAGAGAUGGCACCGUGGUG |
| siRNA 2599 | 2599 | ACCACGGUGCCAUCUCUUG | 5497 | CAAGAGAUGGCACCGUGGU |
| siRNA 2600 | 2600 | CCACGGUGCCAUCUCUUGU | 5498 | ACAAGAGAUGGCACCGUGG |
| siRNA 2601 | 2601 | CACGGUGCCAUCUCUUGUG | 5499 | CACAAGAGAUGGCACCGUG |
| siRNA 2602 | 2602 | ACGGUGCCAUCUCUUGUGU | 5500 | ACACAAGAGAUGGCACCGU |
| siRNA 2603 | 2603 | CGGUGCCAUCUCUUGUGUA | 5501 | UACACAAGAGAUGGCACCG |
| siRNA 2604 | 2604 | GGUGCCAUCUCUUGUGUAU | 5502 | AUACACAAGAGAUGGCACC |
| siRNA 2605 | 2605 | GUGCCAUCUCUUGUGUAUG | 5503 | CAUACACAAGAGAUGGCAC |
| siRNA 2606 | 2606 | UGCCAUCUCUUGUGUAUGC | 5504 | GCAUACACAAGAGAUGGCA |
| siRNA 2607 | 2607 | GCCAUCUCUUGUGUAUGCA | 5505 | UGCAUACACAAGAGAUGGC |
| siRNA 2608 | 2608 | CCAUCUCUUGUGUAUGCAG | 5506 | CUGCAUACACAAGAGAUGG |
| siRNA 2609 | 2609 | CAUCUCUUGUGUAUGCAGG | 5507 | CCUGCAUACACAAGAGAUG |
| siRNA 2610 | 2610 | AUCUCUUGUGUAUGCAGGG | 5508 | CCCUGCAUACACAAGAGAU |
| siRNA 2611 | 2611 | UCUCUUGUGUAUGCAGGGC | 5509 | GCCCUGCAUACACAAGAGA |
| siRNA 2612 | 2612 | CUCUUGUGUAUGCAGGGCU | 5510 | AGCCCUGCAUACACAAGAG |
| siRNA 2613 | 2613 | UCUUGUGUAUGCAGGGCUA | 5511 | UAGCCCUGCAUACACAAGA |
| siRNA 2614 | 2614 | CUUGUGUAUGCAGGGCUAU | 5512 | AUAGCCCUGCAUACACAAG |
| siRNA 2615 | 2615 | UUGUGUAUGCAGGGCUAUG | 5513 | CAUAGCCCUGCAUACACAA |
| siRNA 2616 | 2616 | UGUGUAUGCAGGGCUAUGC | 5514 | GCAUAGCCCUGCAUACACA |
| siRNA 2617 | 2617 | GUGUAUGCAGGGCUAUGCA | 5515 | UGCAUAGCCCUGCAUACAC |
| siRNA 2618 | 2618 | UGUAUGCAGGGCUAUGCAC | 5516 | GUGCAUAGCCCUGCAUACA |
| siRNA 2619 | 2619 | GUAUGCAGGGCUAUGCACC | 5517 | GGUGCAUAGCCCUGCAUAC |
| siRNA 2620 | 2620 | UAUGCAGGGCUAUGCACCU | 5518 | AGGUGCAUAGCCCUGCAUA |
| siRNA 2621 | 2621 | AUGCAGGGCUAUGCACCUG | 5519 | CAGGUGCAUAGCCCUGCAU |
| siRNA 2622 | 2622 | UGCAGGGCUAUGCACCUGC | 5520 | GCAGGUGCAUAGCCCUGCA |
| siRNA 2623 | 2623 | GCAGGGCUAUGCACCUGCA | 5521 | UGCAGGUGCAUAGCCCUGC |
| siRNA 2624 | 2624 | CAGGGCUAUGCACCUGCAG | 5522 | CUGCAGGUGCAUAGCCCUG |
| siRNA 2625 | 2625 | AGGGCUAUGCACCUGCAGG | 5523 | CCUGCAGGUGCAUAGCCCU |
| siRNA 2626 | 2626 | GGGCUAUGCACCUGCAGGC | 5524 | GCCUGCAGGUGCAUAGCCC |
| siRNA 2627 | 2627 | GGCUAUGCACCUGCAGGCA | 5525 | UGCCUGCAGGUGCAUAGCC |
| siRNA 2628 | 2628 | GCUAUGCACCUGCAGGCAC | 5526 | GUGCCUGCAGGUGCAUAGC |
| siRNA 2629 | 2629 | CUAUGCACCUGCAGGCACG | 5527 | CGUGCCUGCAGGUGCAUAG |
| siRNA 2630 | 2630 | UAUGCACCUGCAGGCACGU | 5528 | ACGUGCCUGCAGGUGCAUA |
| siRNA 2631 | 2631 | AUGCACCUGCAGGCACGUG | 5529 | CACGUGCCUGCAGGUGCAU |
| siRNA 2632 | 2632 | UGCACCUGCAGGCACGUGU | 5530 | ACACGUGCCUGCAGGUGCA |
| siRNA 2633 | 2633 | GCACCUGCAGGCACGUGUG | 5531 | CACACGUGCCUGCAGGUGC |
| siRNA 2634 | 2634 | CACCUGCAGGCACGUGUGU | 5532 | ACACACGUGCCUGCAGGUG |
| siRNA 2635 | 2635 | ACCUGCAGGCACGUGUGUA | 5533 | UACACACGUGCCUGCAGGU |
| siRNA 2636 | 2636 | CCUGCAGGCACGUGUGUAU | 5534 | AUACACACGUGCCUGCAGG |
| siRNA 2637 | 2637 | CUGCAGGCACGUGUGUAUG | 5535 | CAUACACACGUGCCUGCAG |
| siRNA 2638 | 2638 | UGCAGGCACGUGUGUAUGC | 5536 | GCAUACACACGUGCCUGCA |
| siRNA 2639 | 2639 | GCAGGCACGUGUGUAUGCA | 5537 | UGCAUACACACGUGCCUGC |
| siRNA 2640 | 2640 | CAGGCACGUGUGUAUGCAC | 5538 | GUGCAUACACACGUGCCUG |
| siRNA 2641 | 2641 | AGGCACGUGUGUAUGCACU | 5539 | AGUGCAUACACACGUGCCU |
| siRNA 2642 | 2642 | GGCACGUGUGUAUGCACUC | 5540 | GAGUGCAUACACACGUGCC |
| siRNA 2643 | 2643 | GCACGUGUGUAUGCACUCC | 5541 | GGAGUGCAUACACACGUGC |
| siRNA 2644 | 2644 | CACGUGUGUAUGCACUCCC | 5542 | GGGAGUGCAUACACACGUG |
| siRNA 2645 | 2645 | ACGUGUGUAUGCACUCCCC | 5543 | GGGGAGUGCAUACACACGU |
| siRNA 2646 | 2646 | CGUGUGUAUGCACUCCCCG | 5544 | CGGGGAGUGCAUACACACG |
| siRNA 2647 | 2647 | GUGUGUAUGCACUCCCCGC | 5545 | GCGGGGAGUGCAUACACAC |
| siRNA 2648 | 2648 | UGUGUAUGCACUCCCCGCU | 5546 | AGCGGGGAGUGCAUACACA |
| siRNA 2649 | 2649 | GUGUAUGCACUCCCCGCUU | 5547 | AAGCGGGGAGUGCAUACAC |
| siRNA 2650 | 2650 | UGUAUGCACUCCCCGCUUG | 5548 | CAAGCGGGGAGUGCAUACA |
| siRNA 2651 | 2651 | GUAUGCACUCCCCGCUUGU | 5549 | ACAAGCGGGGAGUGCAUAC |
| siRNA 2652 | 2652 | UAUGCACUCCCCGCUUGUG | 5550 | CACAAGCGGGGAGUGCAUA |
| siRNA 2653 | 2653 | AUGCACUCCCCGCUUGUGU | 5551 | ACACAAGCGGGGAGUGCAU |
| siRNA 2654 | 2654 | UGCACUCCCCGCUUGUGUU | 5552 | AACACAAGCGGGGAGUGCA |
| siRNA 2655 | 2655 | GCACUCCCCGCUUGUGUUU | 5553 | AAACACAAGCGGGGAGUGC |
| siRNA 2656 | 2656 | CACUCCCCGCUUGUGUUUA | 5554 | UAAACACAAGCGGGGAGUG |
| siRNA 2657 | 2657 | ACUCCCCGCUUGUGUUUAC | 5555 | GUAAACACAAGCGGGGAGU |
| siRNA 2658 | 2658 | CUCCCCGCUUGUGUUUACA | 5556 | UGUAAACACAAGCGGGGAG |
| siRNA 2659 | 2659 | UCCCCGCUUGUGUUUACAC | 5557 | GUGUAAACACAAGCGGGGA |
| siRNA 2660 | 2660 | CCCCGCUUGUGUUUACACA | 5558 | UGUGUAAACACAAGCGGGG |
| siRNA 2661 | 2661 | CCCGCUUGUGUUUACACAA | 5559 | UUGUGUAAACACAAGCGGG |
| siRNA 2662 | 2662 | CCGCUUGUGUUUACACAAG | 5560 | CUUGUGUAAACACAAGCGG |
| siRNA 2663 | 2663 | CGCUUGUGUUUACACAAGC | 5561 | GCUUGUGUAAACACAAGCG |
| siRNA 2664 | 2664 | GCUUGUGUUUACACAAGCU | 5562 | AGCUUGUGUAAACACAAGC |
| siRNA 2665 | 2665 | CUUGUGUUUACACAAGCUG | 5563 | CAGCUUGUGUAAACACAAG |
| siRNA 2666 | 2666 | UUGUGUUUACACAAGCUGU | 5564 | ACAGCUUGUGUAAACACAA |
| siRNA 2667 | 2667 | UGUGUUUACACAAGCUGUG | 5565 | CACAGCUUGUGUAAACACA |
| siRNA 2668 | 2668 | GUGUUUACACAAGCUGUGG | 5566 | CCACAGCUUGUGUAAACAC |
| siRNA 2669 | 2669 | UGUUUACACAAGCUGUGGG | 5567 | CCCACAGCUUGUGUAAACA |
| siRNA 2670 | 2670 | GUUUACACAAGCUGUGGGG | 5568 | CCCCACAGCUUGUGUAAAC |
| siRNA 2671 | 2671 | UUUACACAAGCUGUGGGGU | 5569 | ACCCCACAGCUUGUGUAAA |
| siRNA 2672 | 2672 | UUACACAAGCUGUGGGGUG | 5570 | CACCCCACAGCUUGUGUAA |
| siRNA 2673 | 2673 | UACACAAGCUGUGGGGUGU | 5571 | ACACCCCACAGCUUGUGUA |
| siRNA 2674 | 2674 | ACACAAGCUGUGGGGUGUU | 5572 | AACACCCCACAGCUUGUGU |
| siRNA 2675 | 2675 | CACAAGCUGUGGGGUGUUA | 5573 | UAACACCCCACAGCUUGUG |
| siRNA 2676 | 2676 | ACAAGCUGUGGGGUGUUAC | 5574 | GUAACACCCCACAGCUUGU |
| siRNA 2677 | 2677 | CAAGCUGUGGGGUGUUACG | 5575 | CGUAACACCCCACAGCUUG |
| siRNA 2678 | 2678 | AAGCUGUGGGGUGUUACGC | 5576 | GCGUAACACCCCACAGCUU |
| siRNA 2679 | 2679 | AGCUGUGGGGUGUUACGCA | 5577 | UGCGUAACACCCCACAGCU |
| siRNA 2680 | 2680 | GCUGUGGGGUGUUACGCAU | 5578 | AUGCGUAACACCCCACAGC |
| siRNA 2681 | 2681 | CUGUGGGGUGUUACGCAUG | 5579 | CAUGCGUAACACCCCACAG |
| siRNA 2682 | 2682 | UGUGGGGUGUUACGCAUGC | 5580 | GCAUGCGUAACACCCCACA |
| siRNA 2683 | 2683 | GUGGGGUGUUACGCAUGCC | 5581 | GGCAUGCGUAACACCCCAC |
| siRNA 2684 | 2684 | UGGGGUGUUACGCAUGCCU | 5582 | AGGCAUGCGUAACACCCCA |
| siRNA 2685 | 2685 | GGGGUGUUACGCAUGCCUG | 5583 | CAGGCAUGCGUAACACCCC |
| siRNA 2686 | 2686 | GGGUGUUACGCAUGCCUGC | 5584 | GCAGGCAUGCGUAACACCC |
| siRNA 2687 | 2687 | GGUGUUACGCAUGCCUGCU | 5585 | AGCAGGCAUGCGUAACACC |
| siRNA 2688 | 2688 | GUGUUACGCAUGCCUGCUU | 5586 | AAGCAGGCAUGCGUAACAC |
| siRNA 2689 | 2689 | UGUUACGCAUGCCUGCUUU | 5587 | AAAGCAGGCAUGCGUAACA |
| siRNA 2690 | 2690 | GUUACGCAUGCCUGCUUUU | 5588 | AAAAGCAGGCAUGCGUAAC |
| siRNA 2691 | 2691 | UUACGCAUGCCUGCUUUUU | 5589 | AAAAAGCAGGCAUGCGUAA |
| siRNA 2692 | 2692 | UACGCAUGCCUGCUUUUUU | 5590 | AAAAAAGCAGGCAUGCGUA |
| siRNA 2693 | 2693 | ACGCAUGCCUGCUUUUUUC | 5591 | GAAAAAAGCAGGCAUGCGU |
| siRNA 2694 | 2694 | CGCAUGCCUGCUUUUUUCA | 5592 | UGAAAAAAGCAGGCAUGCG |
| siRNA 2695 | 2695 | GCAUGCCUGCUUUUUUCAC | 5593 | GUGAAAAAAGCAGGCAUGC |
| siRNA 2696 | 2696 | CAUGCCUGCUUUUUUCACU | 5594 | AGUGAAAAAAGCAGGCAUG |
| siRNA 2697 | 2697 | AUGCCUGCUUUUUUCACUU | 5595 | AAGUGAAAAAAGCAGGCAU |
| siRNA 2698 | 2698 | UGCCUGCUUUUUUCACUUA | 5596 | UAAGUGAAAAAAGCAGGCA |
| siRNA 2699 | 2699 | GCCUGCUUUUUUCACUUAA | 5597 | UUAAGUGAAAAAAGCAGGC |
| siRNA 2700 | 2700 | CCUGCUUUUUUCACUUAAU | 5598 | AUUAAGUGAAAAAAGCAGG |
| siRNA 2701 | 2701 | CUGCUUUUUUCACUUAAUA | 5599 | UAUUAAGUGAAAAAAGCAG |
| siRNA 2702 | 2702 | UGCUUUUUUCACUUAAUAA | 5600 | UUAUUAAGUGAAAAAAGCA |
| siRNA 2703 | 2703 | GCUUUUUUCACUUAAUAAU | 5601 | AUUAUUAAGUGAAAAAAGC |
| siRNA 2704 | 2704 | CUUUUUUCACUUAAUAAUA | 5602 | UAUUAUUAAGUGAAAAAAG |
| siRNA 2705 | 2705 | UUUUUUCACUUAAUAAUAC | 5603 | GUAUUAUUAAGUGAAAAAA |
| siRNA 2706 | 2706 | UUUUUCACUUAAUAAUACA | 5604 | UGUAUUAUUAAGUGAAAAA |
| siRNA 2707 | 2707 | UUUUCACUUAAUAAUACAG | 5605 | CUGUAUUAUUAAGUGAAAA |
| siRNA 2708 | 2708 | UUUCACUUAAUAAUACAGC | 5606 | GCUGUAUUAUUAAGUGAAA |
| siRNA 2709 | 2709 | UUCACUUAAUAAUACAGCU | 5607 | AGCUGUAUUAUUAAGUGAA |
| siRNA 2710 | 2710 | UCACUUAAUAAUACAGCUU | 5608 | AAGCUGUAUUAUUAAGUGA |
| siRNA 2711 | 2711 | CACUUAAUAAUACAGCUUG | 5609 | CAAGCUGUAUUAUUAAGUG |
| siRNA 2712 | 2712 | ACUUAAUAAUACAGCUUGG | 5610 | CCAAGCUGUAUUAUUAAGU |
| siRNA 2713 | 2713 | CUUAAUAAUACAGCUUGGA | 5611 | UCCAAGCUGUAUUAUUAAG |
| siRNA 2714 | 2714 | UUAAUAAUACAGCUUGGAG | 5612 | CUCCAAGCUGUAUUAUUAA |
| siRNA 2715 | 2715 | UAAUAAUACAGCUUGGAGA | 5613 | UCUCCAAGCUGUAUUAUUA |
| siRNA 2716 | 2716 | AAUAAUACAGCUUGGAGAG | 5614 | CUCUCCAAGCUGUAUUAUU |
| siRNA 2717 | 2717 | AUAAUACAGCUUGGAGAGA | 5615 | UCUCUCCAAGCUGUAUUAU |
| siRNA 2718 | 2718 | UAAUACAGCUUGGAGAGAU | 5616 | AUCUCUCCAAGCUGUAUUA |
| siRNA 2719 | 2719 | AAUACAGCUUGGAGAGAUU | 5617 | AAUCUCUCCAAGCUGUAUU |
| siRNA 2720 | 2720 | AUACAGCUUGGAGAGAUUU | 5618 | AAAUCUCUCCAAGCUGUAU |
| siRNA 2721 | 2721 | UACAGCUUGGAGAGAUUUU | 5619 | AAAAUCUCUCCAAGCUGUA |
| siRNA 2722 | 2722 | ACAGCUUGGAGAGAUUUUU | 5620 | AAAAAUCUCUCCAAGCUGU |
| siRNA 2723 | 2723 | CAGCUUGGAGAGAUUUUUG | 5621 | CAAAAAUCUCUCCAAGCUG |
| siRNA 2724 | 2724 | AGCUUGGAGAGAUUUUUGU | 5622 | ACAAAAAUCUCUCCAAGCU |
| siRNA 2725 | 2725 | GCUUGGAGAGAUUUUUGUA | 5623 | UACAAAAAUCUCUCCAAGC |
| siRNA 2726 | 2726 | CUUGGAGAGAUUUUUGUAU | 5624 | AUACAAAAAUCUCUCCAAG |
| siRNA 2727 | 2727 | UUGGAGAGAUUUUUGUAUC | 5625 | GAUACAAAAAUCUCUCCAA |
| siRNA 2728 | 2728 | UGGAGAGAUUUUUGUAUCA | 5626 | UGAUACAAAAAUCUCUCCA |
| siRNA 2729 | 2729 | GGAGAGAUUUUUGUAUCAC | 5627 | GUGAUACAAAAAUCUCUCC |
| siRNA 2730 | 2730 | GAGAGAUUUUUGUAUCACA | 5628 | UGUGAUACAAAAAUCUCUC |
| siRNA 2731 | 2731 | AGAGAUUUUUGUAUCACAU | 5629 | AUGUGAUACAAAAAUCUCU |
| siRNA 2732 | 2732 | GAGAUUUUUGUAUCACAUU | 5630 | AAUGUGAUACAAAAAUCUC |
| siRNA 2733 | 2733 | AGAUUUUUGUAUCACAUUA | 5631 | UAAUGUGAUACAAAAAUCU |
| siRNA 2734 | 2734 | GAUUUUUGUAUCACAUUAU | 5632 | AUAAUGUGAUACAAAAAUC |
| siRNA 2735 | 2735 | AUUUUUGUAUCACAUUAUA | 5633 | UAUAAUGUGAUACAAAAAU |
| siRNA 2736 | 2736 | UUUUUGUAUCACAUUAUAA | 5634 | UUAUAAUGUGAUACAAAAA |
| siRNA 2737 | 2737 | UUUUGUAUCACAUUAUAAA | 5635 | UUUAUAAUGUGAUACAAAA |
| siRNA 2738 | 2738 | UUUGUAUCACAUUAUAAAU | 5636 | AUUUAUAAUGUGAUACAAA |
| siRNA 2739 | 2739 | UUGUAUCACAUUAUAAAUC | 5637 | GAUUUAUAAUGUGAUACAA |
| siRNA 2740 | 2740 | UGUAUCACAUUAUAAAUCC | 5638 | GGAUUUAUAAUGUGAUACA |
| siRNA 2741 | 2741 | GUAUCACAUUAUAAAUCCC | 5639 | GGGAUUUAUAAUGUGAUAC |
| siRNA 2742 | 2742 | UAUCACAUUAUAAAUCCCA | 5640 | UGGGAUUUAUAAUGUGAUA |
| siRNA 2743 | 2743 | AUCACAUUAUAAAUCCCAC | 5641 | GUGGGAUUUAUAAUGUGAU |
| siRNA 2744 | 2744 | UCACAUUAUAAAUCCCACU | 5642 | AGUGGGAUUUAUAAUGUGA |
| siRNA 2745 | 2745 | CACAUUAUAAAUCCCACUC | 5643 | GAGUGGGAUUUAUAAUGUG |
| siRNA 2746 | 2746 | ACAUUAUAAAUCCCACUCG | 5644 | CGAGUGGGAUUUAUAAUGU |
| siRNA 2747 | 2747 | CAUUAUAAAUCCCACUCGC | 5645 | GCGAGUGGGAUUUAUAAUG |
| siRNA 2748 | 2748 | AUUAUAAAUCCCACUCGCU | 5646 | AGCGAGUGGGAUUUAUAAU |
| siRNA 2749 | 2749 | UUAUAAAUCCCACUCGCUC | 5647 | GAGCGAGUGGGAUUUAUAA |
| siRNA 2750 | 2750 | UAUAAAUCCCACUCGCUCU | 5648 | AGAGCGAGUGGGAUUUAUA |
| siRNA 2751 | 2751 | AUAAAUCCCACUCGCUCUU | 5649 | AAGAGCGAGUGGGAUUUAU |
| siRNA 2752 | 2752 | UAAAUCCCACUCGCUCUUU | 5650 | AAAGAGCGAGUGGGAUUUA |
| siRNA 2753 | 2753 | AAAUCCCACUCGCUCUUUU | 5651 | AAAAGAGCGAGUGGGAUUU |
| siRNA 2754 | 2754 | AAUCCCACUCGCUCUUUUU | 5652 | AAAAAGAGCGAGUGGGAUU |
| siRNA 2755 | 2755 | AUCCCACUCGCUCUUUUUG | 5653 | CAAAAAGAGCGAGUGGGAU |
| siRNA 2756 | 2756 | UCCCACUCGCUCUUUUUGA | 5654 | UCAAAAAGAGCGAGUGGGA |
| siRNA 2757 | 2757 | CCCACUCGCUCUUUUUGAU | 5655 | AUCAAAAAGAGCGAGUGGG |
| siRNA 2758 | 2758 | CCACUCGCUCUUUUUGAUG | 5656 | CAUCAAAAAGAGCGAGUGG |
| siRNA 2759 | 2759 | CACUCGCUCUUUUUGAUGG | 5657 | CCAUCAAAAAGAGCGAGUG |
| siRNA 2760 | 2760 | ACUCGCUCUUUUUGAUGGC | 5658 | GCCAUCAAAAAGAGCGAGU |
| siRNA 2761 | 2761 | CUCGCUCUUUUUGAUGGCC | 5659 | GGCCAUCAAAAAGAGCGAG |
| siRNA 2762 | 2762 | UCGCUCUUUUUGAUGGCCA | 5660 | UGGCCAUCAAAAAGAGCGA |
| SIRNA 2763 | 2763 | CGCUCUUUUUGAUGGCCAC | 5661 | GUGGCCAUCAAAAAGAGCG |
| siRNA 2764 | 2764 | GCUCUUUUUGAUGGCCACA | 5662 | UGUGGCCAUCAAAAAGAGC |
| siRNA 2765 | 2765 | CUCUUUUUGAUGGCCACAU | 5663 | AUGUGGCCAUCAAAAAGAG |
| siRNA 2766 | 2766 | UCUUUUUGAUGGCCACAUA | 5664 | UAUGUGGCCAUCAAAAAGA |
| siRNA 2767 | 2767 | CUUUUUGAUGGCCACAUAA | 5665 | UUAUGUGGCCAUCAAAAAG |
| siRNA 2768 | 2768 | UUUUUGAUGGCCACAUAAU | 5666 | AUUAUGUGGCCAUCAAAAA |
| siRNA 2769 | 2769 | UUUUGAUGGCCACAUAAUA | 5667 | UAUUAUGUGGCCAUCAAAA |
| siRNA 2770 | 2770 | UUUGAUGGCCACAUAAUAA | 5668 | UUAUUAUGUGGCCAUCAAA |
| siRNA 2771 | 2771 | UUGAUGGCCACAUAAUAAC | 5669 | GUUAUUAUGUGGCCAUCAA |
| siRNA 2772 | 2772 | UGAUGGCCACAUAAUAACU | 5670 | AGUUAUUAUGUGGCCAUCA |
| siRNA 2773 | 2773 | GAUGGCCACAUAAUAACUA | 5671 | UAGUUAUUAUGUGGCCAUC |
| siRNA 2774 | 2774 | AUGGCCACAUAAUAACUAC | 5672 | GUAGUUAUUAUGUGGCCAU |
| siRNA 2775 | 2775 | UGGCCACAUAAUAACUACU | 5673 | AGUAGUUAUUAUGUGGCCA |
| siRNA 2776 | 2776 | GGCCACAUAAUAACUACUG | 5674 | CAGUAGUUAUUAUGUGGCC |
| siRNA 2777 | 2777 | GCCACAUAAUAACUACUGC | 5675 | GCAGUAGUUAUUAUGUGGC |
| siRNA 2778 | 2778 | CCACAUAAUAACUACUGCA | 5676 | UGCAGUAGUUAUUAUGUGG |
| siRNA 2779 | 2779 | CACAUAAUAACUACUGCAU | 5677 | AUGCAGUAGUUAUUAUGUG |
| siRNA 2780 | 2780 | ACAUAAUAACUACUGCAUA | 5678 | UAUGCAGUAGUUAUUAUGU |
| siRNA 2781 | 2781 | CAUAAUAACUACUGCAUAA | 5679 | UUAUGCAGUAGUUAUUAUG |
| siRNA 2782 | 2782 | AUAAUAACUACUGCAUAAU | 5680 | AUUAUGCAGUAGUUAUUAU |
| siRNA 2783 | 2783 | UAAUAACUACUGCAUAAUA | 5681 | UAUUAUGCAGUAGUUAUUA |
| siRNA 2784 | 2784 | AAUAACUACUGCAUAAUAU | 5682 | AUAUUAUGCAGUAGUUAUU |
| siRNA 2785 | 2785 | AUAACUACUGCAUAAUAUG | 5683 | CAUAUUAUGCAGUAGUUAU |
| siRNA 2786 | 2786 | UAACUACUGCAUAAUAUGG | 5684 | CCAUAUUAUGCAGUAGUUA |
| siRNA 2787 | 2787 | AACUACUGCAUAAUAUGGA | 5685 | UCCAUAUUAUGCAGUAGUU |
| siRNA 2788 | 2788 | ACUACUGCAUAAUAUGGAU | 5686 | AUCCAUAUUAUGCAGUAGU |
| siRNA 2789 | 2789 | CUACUGCAUAAUAUGGAUA | 5687 | UAUCCAUAUUAUGCAGUAG |
| siRNA 2790 | 2790 | UACUGCAUAAUAUGGAUAC | 5688 | GUAUCCAUAUUAUGCAGUA |
| siRNA 2791 | 2791 | ACUGCAUAAUAUGGAUACG | 5689 | CGUAUCCAUAUUAUGCAGU |
| siRNA 2792 | 2792 | CUGCAUAAUAUGGAUACGC | 5690 | GCGUAUCCAUAUUAUGCAG |
| siRNA 2793 | 2793 | UGCAUAAUAUGGAUACGCC | 5691 | GGCGUAUCCAUAUUAUGCA |
| siRNA 2794 | 2794 | GCAUAAUAUGGAUACGCCU | 5692 | AGGCGUAUCCAUAUUAUGC |
| siRNA 2795 | 2795 | CAUAAUAUGGAUACGCCUU | 5693 | AAGGCGUAUCCAUAUUAUG |
| siRNA 2796 | 2796 | AUAAUAUGGAUACGCCUUA | 5694 | UAAGGCGUAUCCAUAUUAU |
| siRNA 2797 | 2797 | UAAUAUGGAUACGCCUUAU | 5695 | AUAAGGCGUAUCCAUAUUA |
| siRNA 2798 | 2798 | AAUAUGGAUACGCCUUAUU | 5696 | AAUAAGGCGUAUCCAUAUU |
| siRNA 2799 | 2799 | AUAUGGAUACGCCUUAUUU | 5697 | AAAUAAGGCGUAUCCAUAU |
| siRNA 2800 | 2800 | UAUGGAUACGCCUUAUUUG | 5698 | CAAAUAAGGCGUAUCCAUA |
| siRNA 2801 | 2801 | AUGGAUACGCCUUAUUUGA | 5699 | UCAAAUAAGGCGUAUCCAU |
| siRNA 2802 | 2802 | UGGAUACGCCUUAUUUGAU | 5700 | AUCAAAUAAGGCGUAUCCA |
| siRNA 2803 | 2803 | GGAUACGCCUUAUUUGAUU | 5701 | AAUCAAAUAAGGCGUAUCC |
| siRNA 2804 | 2804 | GAUACGCCUUAUUUGAUUU | 5702 | AAAUCAAAUAAGGCGUAUC |
| siRNA 2805 | 2805 | AUACGCCUUAUUUGAUUUA | 5703 | UAAAUCAAAUAAGGCGUAU |
| siRNA 2806 | 2806 | UACGCCUUAUUUGAUUUAA | 5704 | UUAAAUCAAAUAAGGCGUA |
| siRNA 2807 | 2807 | ACGCCUUAUUUGAUUUAAC | 5705 | GUUAAAUCAAAUAAGGCGU |
| siRNA 2808 | 2808 | CGCCUUAUUUGAUUUAACU | 5706 | AGUUAAAUCAAAUAAGGCG |
| siRNA 2809 | 2809 | GCCUUAUUUGAUUUAACUA | 5707 | UAGUUAAAUCAAAUAAGGC |
| siRNA 2810 | 2810 | CCUUAUUUGAUUUAACUAG | 5708 | CUAGUUAAAUCAAAUAAGG |
| siRNA 2811 | 2811 | CUUAUUUGAUUUAACUAGU | 5709 | ACUAGUUAAAUCAAAUAAG |
| siRNA 2812 | 2812 | UUAUUUGAUUUAACUAGUU | 5710 | AACUAGUUAAAUCAAAUAA |
| siRNA 2813 | 2813 | UAUUUGAUUUAACUAGUUC | 5711 | GAACUAGUUAAAUCAAAUA |
| siRNA 2814 | 2814 | AUUUGAUUUAACUAGUUCC | 5712 | GGAACUAGUUAAAUCAAAU |
| siRNA 2815 | 2815 | UUUGAUUUAACUAGUUCCC | 5713 | GGGAACUAGUUAAAUCAAA |
| siRNA 2816 | 2816 | UUGAUUUAACUAGUUCCCU | 5714 | AGGGAACUAGUUAAAUCAA |
| siRNA 2817 | 2817 | UGAUUUAACUAGUUCCCUA | 5715 | UAGGGAACUAGUUAAAUCA |
| siRNA 2818 | 2818 | GAUUUAACUAGUUCCCUAA | 5716 | UUAGGGAACUAGUUAAAUC |
| siRNA 2819 | 2819 | AUUUAACUAGUUCCCUAAU | 5717 | AUUAGGGAACUAGUUAAAU |
| siRNA 2820 | 2820 | UUUAACUAGUUCCCUAAUG | 5718 | CAUUAGGGAACUAGUUAAA |
| siRNA 2821 | 2821 | UUAACUAGUUCCCUAAUGA | 5719 | UCAUUAGGGAACUAGUUAA |
| siRNA 2822 | 2822 | UAACUAGUUCCCUAAUGAU | 5720 | AUCAUUAGGGAACUAGUUA |
| siRNA 2823 | 2823 | AACUAGUUCCCUAAUGAUG | 5721 | CAUCAUUAGGGAACUAGUU |
| siRNA 2824 | 2824 | ACUAGUUCCCUAAUGAUGG | 5722 | CCAUCAUUAGGGAACUAGU |
| siRNA 2825 | 2825 | CUAGUUCCCUAAUGAUGGA | 5723 | UCCAUCAUUAGGGAACUAG |
| siRNA 2826 | 2826 | UAGUUCCCUAAUGAUGGAC | 5724 | GUCCAUCAUUAGGGAACUA |
| siRNA 2827 | 2827 | AGUUCCCUAAUGAUGGACU | 5725 | AGUCCAUCAUUAGGGAACU |
| siRNA 2828 | 2828 | GUUCCCUAAUGAUGGACUU | 5726 | AAGUCCAUCAUUAGGGAAC |
| siRNA 2829 | 2829 | UUCCCUAAUGAUGGACUUU | 5727 | AAAGUCCAUCAUUAGGGAA |
| siRNA 2830 | 2830 | UCCCUAAUGAUGGACUUUU | 5728 | AAAAGUCCAUCAUUAGGGA |
| siRNA 2831 | 2831 | CCCUAAUGAUGGACUUUUA | 5729 | UAAAAGUCCAUCAUUAGGG |
| siRNA 2832 | 2832 | CCUAAUGAUGGACUUUUAA | 5730 | UUAAAAGUCCAUCAUUAGG |
| siRNA 2833 | 2833 | CUAAUGAUGGACUUUUAAG | 5731 | CUUAAAAGUCCAUCAUUAG |
| siRNA 2834 | 2834 | UAAUGAUGGACUUUUAAGU | 5732 | ACUUAAAAGUCCAUCAUUA |
| siRNA 2835 | 2835 | AAUGAUGGACUUUUAAGUU | 5733 | AACUUAAAAGUCCAUCAUU |
| siRNA 2836 | 2836 | AUGAUGGACUUUUAAGUUG | 5734 | CAACUUAAAAGUCCAUCAU |
| siRNA 2837 | 2837 | UGAUGGACUUUUAAGUUGU | 5735 | ACAACUUAAAAGUCCAUCA |
| siRNA 2838 | 2838 | GAUGGACUUUUAAGUUGUU | 5736 | AACAACUUAAAAGUCCAUC |
| siRNA 2839 | 2839 | AUGGACUUUUAAGUUGUUU | 5737 | AAACAACUUAAAAGUCCAU |
| siRNA 2840 | 2840 | UGGACUUUUAAGUUGUUUC | 5738 | GAAACAACUUAAAAGUCCA |
| siRNA 2841 | 2841 | GGACUUUUAAGUUGUUUCC | 5739 | GGAAACAACUUAAAAGUCC |
| siRNA 2842 | 2842 | GACUUUUAAGUUGUUUCCU | 5740 | AGGAAACAACUUAAAAGUC |
| siRNA 2843 | 2843 | ACUUUUAAGUUGUUUCCUU | 5741 | AAGGAAACAACUUAAAAGU |
| siRNA 2844 | 2844 | CUUUUAAGUUGUUUCCUUU | 5742 | AAAGGAAACAACUUAAAAG |
| siRNA 2845 | 2845 | UUUUAAGUUGUUUCCUUUU | 5743 | AAAAGGAAACAACUUAAAA |
| siRNA 2846 | 2846 | UUUAAGUUGUUUCCUUUUU | 5744 | AAAAAGGAAACAACUUAAA |
| siRNA 2847 | 2847 | UUAAGUUGUUUCCUUUUUU | 5745 | AAAAAAGGAAACAACUUAA |
| siRNA 2848 | 2848 | UAAGUUGUUUCCUUUUUUU | 5746 | AAAAAAAGGAAACAACUUA |
| siRNA 2849 | 2849 | AAGUUGUUUCCUUUUUUUU | 5747 | AAAAAAAAGGAAACAACUU |
| siRNA 2850 | 2850 | AGUUGUUUCCUUUUUUUUU | 5748 | AAAAAAAAAGGAAACAACU |
| siRNA 2851 | 2851 | GUUGUUUCCUUUUUUUUUC | 5749 | GAAAAAAAAAGGAAACAAC |
| siRNA 2852 | 2852 | UUGUUUCCUUUUUUUUUCU | 5750 | AGAAAAAAAAAGGAAACAA |
| siRNA 2853 | 2853 | UGUUUCCUUUUUUUUUCUU | 5751 | AAGAAAAAAAAAGGAAACA |
| siRNA 2854 | 2854 | GUUUCCUUUUUUUUUCUUU | 5752 | AAAGAAAAAAAAAGGAAAC |
| siRNA 2855 | 2855 | UUUCCUUUUUUUUUCUUUU | 5753 | AAAAGAAAAAAAAAGGAAA |
| siRNA 2856 | 2856 | UUCCUUUUUUUUUCUUUUU | 5754 | AAAAAGAAAAAAAAAGGAA |
| siRNA 2857 | 2857 | UCCUUUUUUUUUCUUUUUU | 5755 | AAAAAAGAAAAAAAAAGGA |
| siRNA 2858 | 2858 | CCUUUUUUUUUCUUUUUUG | 5756 | CAAAAAAGAAAAAAAAAGG |
| siRNA 2859 | 2859 | CUUUUUUUUUCUUUUUUGC | 5757 | GCAAAAAAGAAAAAAAAAG |
| siRNA 2860 | 2860 | UUUUUUUUUCUUUUUUGCU | 5758 | AGCAAAAAAGAAAAAAAAA |
| siRNA 2861 | 2861 | UUUUUUUUCUUUUUUGCUA | 5759 | UAGCAAAAAAGAAAAAAAA |
| siRNA 2862 | 2862 | UUUUUUUCUUUUUUGCUAC | 5760 | GUAGCAAAAAAGAAAAAAA |
| siRNA 2863 | 2863 | UUUUUUCUUUUUUGCUACU | 5761 | AGUAGCAAAAAAGAAAAAA |
| siRNA 2864 | 2864 | UUUUUCUUUUUUGCUACUG | 5762 | CAGUAGCAAAAAAGAAAAA |
| siRNA 2865 | 2865 | UUUUCUUUUUUGCUACUGC | 5763 | GCAGUAGCAAAAAAGAAAA |
| siRNA 2866 | 2866 | UUUCUUUUUUGCUACUGCA | 5764 | UGCAGUAGCAAAAAAGAAA |
| siRNA 2867 | 2867 | UUCUUUUUUGCUACUGCAA | 5765 | UUGCAGUAGCAAAAAAGAA |
| siRNA 2868 | 2868 | UCUUUUUUGCUACUGCAAA | 5766 | UUUGCAGUAGCAAAAAAGA |
| siRNA 2869 | 2869 | CUUUUUUGCUACUGCAAAC | 5767 | GUUUGCAGUAGCAAAAAAG |
| siRNA 2870 | 2870 | UUUUUUGCUACUGCAAACG | 5768 | CGUUUGCAGUAGCAAAAAA |
| siRNA 2871 | 2871 | UUUUUGCUACUGCAAACGA | 5769 | UCGUUUGCAGUAGCAAAAA |
| siRNA 2872 | 2872 | UUUUGCUACUGCAAACGAU | 5770 | AUCGUUUGCAGUAGCAAAA |
| siRNA 2873 | 2873 | UUUGCUACUGCAAACGAUG | 5771 | CAUCGUUUGCAGUAGCAAA |
| siRNA 2874 | 2874 | UUGCUACUGCAAACGAUGC | 5772 | GCAUCGUUUGCAGUAGCAA |
| siRNA 2875 | 2875 | UGCUACUGCAAACGAUGCU | 5773 | AGCAUCGUUUGCAGUAGCA |
| siRNA 2876 | 2876 | GCUACUGCAAACGAUGCUA | 5774 | UAGCAUCGUUUGCAGUAGC |
| siRNA 2877 | 2877 | CUACUGCAAACGAUGCUAU | 5775 | AUAGCAUCGUUUGCAGUAG |
| siRNA 2878 | 2878 | UACUGCAAACGAUGCUAUA | 5776 | UAUAGCAUCGUUUGCAGUA |
| siRNA 2879 | 2879 | ACUGCAAACGAUGCUAUAA | 5777 | UUAUAGCAUCGUUUGCAGU |
| siRNA 2880 | 2880 | CUGCAAACGAUGCUAUAAU | 5778 | AUUAUAGCAUCGUUUGCAG |
| siRNA 2881 | 2881 | UGCAAACGAUGCUAUAAUA | 5779 | UAUUAUAGCAUCGUUUGCA |
| siRNA 2882 | 2882 | GCAAACGAUGCUAUAAUAA | 5780 | UUAUUAUAGCAUCGUUUGC |
| siRNA 2883 | 2883 | CAAACGAUGCUAUAAUAAA | 5781 | UUUAUUAUAGCAUCGUUUG |
| siRNA 2884 | 2884 | AAACGAUGCUAUAAUAAAU | 5782 | AUUUAUUAUAGCAUCGUUU |
| siRNA 2885 | 2885 | AACGAUGCUAUAAUAAAUG | 5783 | CAUUUAUUAUAGCAUCGUU |
| siRNA 2886 | 2886 | ACGAUGCUAUAAUAAAUGU | 5784 | ACAUUUAUUAUAGCAUCGU |
| siRNA 2887 | 2887 | CGAUGCUAUAAUAAAUGUC | 5785 | GACAUUUAUUAUAGCAUCG |
| siRNA 2888 | 2888 | GAUGCUAUAAUAAAUGUCC | 5786 | GGACAUUUAUUAUAGCAUC |
| siRNA 2889 | 2889 | AUGCUAUAAUAAAUGUCCU | 5787 | AGGACAUUUAUUAUAGCAU |
| siRNA 2890 | 2890 | UGCUAUAAUAAAUGUCCUU | 5788 | AAGGACAUUUAUUAUAGCA |
| siRNA 2891 | 2891 | GCUAUAAUAAAUGUCCUUA | 5789 | UAAGGACAUUUAUUAUAGC |
| siRNA 2892 | 2892 | CUAUAAUAAAUGUCCUUAU | 5790 | AUAAGGACAUUUAUUAUAG |
| siRNA 2893 | 2893 | UAUAAUAAAUGUCCUUAUC | 5791 | GAUAAGGACAUUUAUUAUA |
| siRNA 2894 | 2894 | AUAAUAAAUGUCCUUAUCA | 5792 | UGAUAAGGACAUUUAUUAU |
| siRNA 2895 | 2895 | UAAUAAAUGUCCUUAUCAA | 5793 | UUGAUAAGGACAUUUAUUA |
| siRNA 2896 | 2896 | AAUAAAUGUCCUUAUCAAA | 5794 | UUUGAUAAGGACAUUUAUU |
| siRNA 2897 | 2897 | AUAAAUGUCCUUAUCAAAA | 5795 | UUUUGAUAAGGACAUUUAU |
| siRNA 2898 | 2898 | UAAAUGUCCUUAUCAAAAA | 5796 | UUUUUGAUAAGGACAUUUA |

Additional Sequence

| SEQ ID | |
|---|---|
| NO: | 5′ to 3′ Sequence |
| 5895 | ACTCTGCAGCCTGGGCTCTGTGAGACTGAGG |
| | TGGCGGTCAGCCGGAGTGAGTGTTGGGGTC |
| | CTGGGGCACCTGCCTTACATGGCTTGTTTA |
| | TGAACATTAAAGGGAAGAAGTTGAAGCTTG |
| | AGGAGCGAGGATGGCAGTCAACAAAGGCCT |
| | CACCTTGCTGGATGGAGACCTCCCTGAGCA |
| | GGAGAATGTGCTGCAGCGGGTCCTGCAGCT |
| | GCCGGTGGTGAGTGGCACCTGCGAATGCTT |
| | CCAGAAGACCTACACCAGCACTAAGGAAGC |
| | CCACCCCCTGGTGGCCTCTGTGTGCAATGC |
| | CTATGAGAAGGGCGTGCAGAGCGCCAGTAG |
| | CTTGGCTGCCTGGAGCATGGAGCCGGTGGT |
| | CCGCAGGCTGTCCACCCAGTTCACAGCTGC |
| | CAATGAGCTGGCCTGCCGAGGCTTGGACCA |
| | CCTGGAGGAAAAGATCCCCGCCCTCCAGTA |
| | CCCCCCTGAAAAGATTGCTTCTGAGCTGAA |
| | GGACACCATCTCCACCCGCCTCCGCAGTGC |
| | CAGAAACAGCATCAGCGTTCCCATCGCGAG |
| | CACTTCAGACAAGGTCCTGGGGGCCGCTTT |
| | GGCCGGGTGCGAGCTTGCCTGGGGGGTGGC |
| | CAGAGACACTGCGGAATTTGCTGCCAACAC |
| | TCGAGCTGGCCGACTGGCTTCTGGAGGGGC |
| | CGACTTGGCCTTGGGCAGCATTGAGAAGGT |
| | GGTGGAGTACCTCCTCCCTCCAGACAAGGA |
| | AGAGTCAGCCCCTGCTCCTGGACACCAGCA |
| | AGCCCAGAAGTCTCCCAAGGCCAAGCCAAG |
| | CCTCTTGAGCAGGGTTGGGGCTCTGACCAA |
| | CACCCTCTCTCGATACACCGTGCAGACCAT |
| | GGCCCGGGCCCTGGAGCAGGGCCACACCGT |
| | GGCCATGTGGATCCCAGGCGTGGTGCCCCT |
| | GAGCAGCCTGGCCCAGTGGGGTGCCTCAGT |
| | GGCCATGCAGGCGGTGTCCCGGCGGAGGAG |
| | CGAAGTGCGGGTACCCTGGCTGCACAGCCT |
| | CGCAGCCGCCCAGGAGGAGGATCATGAGGA |
| | CCAGACAGACACGGAGGGAGAGGACACGGA |
| | GGAGGAGGAAGAATTGGAGACTGAGGAGAA |
| | CAAGTTCAGTGAGGTAGCAGCCCTGCCAGG |
| | CCCTCGAGGCCTCCTGGGTGGTGTGGCACA |
| | TACCCTGCAGAAGACCCTCCAGACCACCAT |
| | CTCGGCTGTGACATGGGCACCTGCAGCTGT |
| | GCTGGGCATGGCAGGGAGGGTGCTGCACCT |
| | CACACCAGCCCCTGCTGTCTCCTCAACCAA |
| | GGGGAGGGCCATGTCCCTATCAGATGCCCT |
| | GAAGGGCGTTACTGACAACGTGGTGGACAC |
| | AGTGGTGCATTACGTGCCGCTCCCCAGGCT |
| | GTCGCTGATGGAGCCCGAGAGCGAATTCCG |
| | GGACATCGACAACCCACCAGCCGAGGTCGA |
| | GCGCCGGGAGGCGGAGCGCAGAGCGTCTGG |
| | GGCGCCGTCCGCCGGCCCGGAGCCCGCCCC |
| | GCGTCTCGCACAGCCCCGCCGCAGCCTGCG |
| | CAGCGCGCAGAGCCCCGGCGCGCCCCCCGG |
| | CCCGGGCCTGGAGGACGAAGTCGCCACGCC |
| | CGCAGCGCCGCGCCCGGGCTTCCCGGCCGT |
| | GCCCCGCGAGAAGCCAAAGCGCAGGGTCAG |
| | CGACAGCTTCTTCCGGCCCAGCGTCATGGA |
| | GCCCATCCTGGGCCGCACGCATTACAGCCA |
| | GCTGCGCAAGAAGAGCTGAGTCGCCGCACC |
| | AGCCGCCGCGCCCCGGGCCGGCGGGTTTCT |
| | CTAACAAATAAACAGAACCCGCACTGCCCA |
| | GGCGAGCGTTGCCACTTTCAAAGTGGTCCC |
| | CTGGGGAGCTCAGCCTCATCCTGATGATGC |
| | TGCCAAGGCGCACTTTTTATTTTTATTTTA |
| | TTTTTATTTTTTTTTTAGCATCCTTTTGGG |
| | GCTTCACTCTCAGAGCCAGTTTTTAAGGGA |
| | CACCAGAGCCGCAGCCTGCTCTGATTCTAT |
| | GGCTTGGTTGTTACTATAAGAGTAATTGCC |
| | TAACTTGATTTTTCATCTCTTTAACCAAAC |
| | TTGTGGCCAAAAGATATTTGACCGTTTCCA |
| | AAATTCAGATTCTGCCTCTGCGGATAAATA |
| | TTTGCCACGAATGAGTAACTCCTGTCACCA |
| | CTCTGAAGGTCCAGACAGAAGGTTTTGACA |
| | CATTCTTAGCACTGAACTCCTCTGTGATCT |
| | AGGATGATCTGTTCCCCCTCTGATGAACAT |
| | CCTCTGATGATCTAGGCTCCCAGCAGGCTA |
| | CTTTGAAGGGAACAATCAGATGCAAAAGCT |
| | CTTGGGTGTTTATTTAAAATACTAGTGTCA |
| | CTTTCTGAGTACCCGCCGCTTCACAGGCTG |
| | AGTCCAGGCCTGTGTGCTTTGTAGAGCCAG |
| | CTGCTTGCTCACAGCCACATTTCCATTTGC |
| | ATCATTACTGCCTTCACCTGCATAGTCACT |
| | CTTTTGATGCTGGGGAACCAAAATGGTGAT |
| | GATATATAGACTTTATGTATAGCCACAGTT |
| | CATCCCCAACCCTAGTCTTCGAAATGTTAA |
| | TATTTGATAAATCTAGAAAATGCATTCATA |
| | CAATTACAGAATTCAAATATTGCAAAAGGA |
| | TGTGTGTCTTTCTCCCCGAGCTCCCCTGTT |
| | CCCCTTCATTGAAAACCACCACGGTGCCAT |
| | CTCTTGTGTATGCAGGGCTATGCACCTGCA |
| | GGCACGTGTGTATGCACTCCCCGCTTGTGT |
| | TTACACAAGCTGTGGGGTGTTACGCATGCC |
| | TGCTTTTTTCACTTAATAATACAGCTTGGA |
| | GAGATTTTTGTATCACATTATAAATCCCAC |
| | TCGCTCTTTTTGATGGCCACATAATAACTA |
| | CTGCATAATATGGATACGCCTTATTTGATT |
| | TAACTAGTTCCCTAATGATGGACTTTTAAG |
| | TTGTTTCCTTTTTTTTTCTTTTTTGCTACT |
| | GCAAACGATGCTATAATAAATGTCCTTATC |
| | AAAAA |

What is claimed is:

1. A composition comprising an oligonucleotide that targets PLIN1 and when administered to a subject in an effective amount decreases circulating cholesterol, triglycerides, APOB, glucose, hemoglobin A1c, alanine aminotransferase, aspartate aminotransferase, alkaline phosphatase, or gamma-glutamyl transferase in a subject.

2. The composition of claim 1, wherein the cholesterol comprises total cholesterol, low density lipoprotein cholesterol, or non-high density lipoprotein cholesterol.

3. The composition of claim 1, wherein the decrease is by about 10% or more, as compared to prior to administration.

4. A composition comprising an oligonucleotide that targets PLIN1 and when administered to a subject in an effective amount decreases systolic blood pressure or diastolic blood pressure in a subject.

5. The composition of claim 4, wherein the decrease is by about 10% or more, as compared to prior to administration.

6. A composition comprising an oligonucleotide that targets PLIN1 and when administered to a subject in an effective amount decreases a liver fibrosis score, non-alcoholic fatty liver disease (NAFLD) fibrosis score, NAFLD activity score, or liver fat percentage in a subject.

7. The composition of claim 6, wherein the decrease is by about 10% or more, as compared to prior to administration.

8. A composition comprising an oligonucleotide that targets PLIN1 and when administered to a subject in an effective amount increases circulating high density lipoprotein cholesterol or apolipoprotein A1 in a subject.

9. The composition of claim 8, wherein the increase is by about 10% or more, as compared to prior to administration.

10. A composition comprising an oligonucleotide that targets PLIN1 and when administered to a subject in an effective amount increases left ventricular ejection fraction in a subject.

11. The composition of claim 10, wherein the increase is by about 10% or more, as compared to prior to administration.

12. A composition comprising an oligonucleotide that targets PLIN1 and when administered to a subject in an effective amount increases insulin sensitivity in a subject.

13. The composition of claim 12, wherein the increase is by about 10% or more, as compared to prior to administration.

14. The composition of any one of claims 1-13, wherein the oligonucleotide comprises a modified internucleoside linkage.

15. The composition of claim 14, wherein the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof.

16. The composition of claim 14, wherein the modified internucleoside linkage comprises one or more phosphorothioate linkages.

17. The composition of any one of claims 1-13, wherein the oligonucleotide comprises 1, 2, 3, 4, 5, 6, 7, 8, 9, 10, 11, 12, 13, 14, 15, 16, 17, 18, 19, or 20 modified internucleoside linkages.

18. The composition of any one of claims 1-13, wherein the oligonucleotide comprises a modified nucleoside.

19. The composition of claim 18, wherein the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof.

20. The composition of claim 18, wherein the modified nucleoside comprises a LNA.

21. The composition of claim 18, wherein the modified nucleoside comprises a 2′,4″ constrained ethyl nucleic acid.

22. The composition of claim 18, wherein the modified nucleoside comprises a 2′-O-methyl nucleoside, 2′-deoxy fluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof.

23. The composition of claim 18, wherein the modified nucleoside comprises one or more 2 fluoro modified nucleosides.

24. The composition of claim 18, wherein the modified nucleoside comprises a 2′ O-alkyl modified nucleoside.

25. The composition of any one of claims 1-13, wherein the oligonucleotide comprises 1, 2, 3, 4, 5, 6, 7, 8, 9, 10, 11, 12, 13, 14, 15, 16, 17, 18, 19, 20, or 21 modified nucleosides.

26. The composition of any one of claims 1-13, wherein the oligonucleotide comprises a lipid attached at a 3′ or 5′ terminus of the oligonucleotide.

27. The composition of claim 26, wherein the lipid comprises cholesterol, myristoyl, palmitoyl, stearoyl, lithocholyl, docosanoyl, docosahexaenoyl, myristyl, palmityl stearyl, or α-tocopherol, or a combination thereof.

28. The composition of any one of claims 1-13, wherein the oligonucleotide comprises a sugar moiety attached at a 3′ or 5′ terminus of the oligonucleotide.

29. The composition of claim 28, wherein the sugar comprises N-acetylgalactosamine (GalNAc), N-acetylglucosamine (GlcNAc), or mannose.

30. The composition of any one of claims 1-13, wherein the oligonucleotide comprises a small interfering RNA (siRNA) comprising a sense strand and an antisense strand.

31. The composition of claim 30, wherein the sense strand is 12-30 nucleosides in length.

32. The composition of claim 30, wherein the antisense strand is 12-30 nucleosides in length.

33. A composition comprising an oligonucleotide that inhibits the expression of PLIN1, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of SEQ ID NO: 6014.

34. The composition of claim 30, wherein any one of the following is true with regard to the sense strand:
all purines comprise 2′ fluoro modified purines, and all pyrimidines comprise a mixture of 2′ fluoro and 2′ methyl modified pyrimidines:
all purines comprise 2′ methyl modified purines, and all pyrimidines comprise a mixture of 2′ fluoro and 2′ methyl modified pyrimidines:
all purines comprise 2′ fluoro modified purines, and all pyrimidines comprise 2′ methyl modified pyrimidines:
all pyrimidines comprise 2′ fluoro modified pyrimidines, and all purines comprise a mixture of 2′ fluoro and 2′ methyl modified purines:
all pyrimidines comprise 2′ methyl modified pyrimidines, and all purines comprise a mixture of 2′ fluoro and 2′ methyl modified purines; or
all pyrimidines comprise 2′ fluoro modified pyrimidines, and all purines comprise 2′ methyl modified purines.

35. The composition of claim 30, wherein any one of the following is true with regard to the antisense strand:
all purines comprise 2′ fluoro modified purines, and all pyrimidines comprise a mixture of 2′ fluoro and 2′ methyl modified pyrimidines:
all purines comprise 2′ methyl modified purines, and all pyrimidines comprise a mixture of 2′ fluoro and 2′ methyl modified pyrimidines:
all purines comprise 2′ methyl modified purines, and all pyrimidines comprise 2′ fluoro modified pyrimidines:
all pyrimidines comprise 2′ fluoro modified pyrimidines, and all purines comprise a mixture of 2′ fluoro and 2′ methyl modified purines:
all pyrimidines comprise 2′ methyl modified pyrimidines, and all purines comprise a mixture of 2′ fluoro and 2′ methyl modified purines; or
all pyrimidines comprise 2′ methyl modified pyrimidines, and all purines comprise 2′ fluoro modified purines.

36. The composition of any one of claims 1-13, wherein the oligonucleotide comprises an antisense oligonucleotide (ASO).

37. The composition of claim 36, wherein the ASO is 12-30 nucleosides in length.

38. A composition comprising an oligonucleotide that inhibits the expression of PLIN1, wherein the oligonucleotide comprises an ASO about 12-30 nucleosides in length and a nucleoside sequence complementary to about 12-30 contiguous nucleosides of SEQ ID NO: 6014.

39. The composition of any one of claims 1-13, further comprising a pharmaceutically acceptable carrier.

40. A method of treating a subject having a cardiometabolic disorder, comprising administering an effective amount of the composition of claim 39 to the subject.

41. The method of claim 40, wherein the cardiometabolic disorder comprises hyperlipidemia, hypertriglyceridemia, cardiovascular disease, coronary artery disease, myocardial infarction, heart failure, cerebrovascular disease, stroke, peripheral vascular disease, peripheral arterial disease, hypertension, diabetes, NAFLD, or non-alcoholic steatohepatitis.
